(19) United States
(12) Patent Application Publication
(10) Pub. No.: US 2011/0117545 A1
(43) Pub. Date: May 19, 2011

(54) GENETIC VARIANTS ON CHR2 AND CHR16 AS MARKERS FOR USE IN BREAST CANCER RISK ASSESSMENT, DIAGNOSIS, PROGNOSIS AND TREATMENT

(75) Inventors: Simon Stacey, Kopavogur (IS); Patrick Sulem, Reykjavik (IS); Andrei Manolescu, Reykjavik (IS)

(73) Assignee: deCODE Genetics ehf, Reykjavik (IS)

(21) Appl. No.: 12/302,473

(22) PCT Filed: Mar. 26, 2008
(86) PCT No.: PCT/IS2008/000009

§ 371 Date: Nov. 25, 2008

(30) Foreign Application Priority Data
Mar. 26, 2007 (IS) 8625
May 25, 2007 (IS) 8648

(51) Int. Cl. C12Q 1/68; C12M 1/34

(52) U.S. Cl. 435/6; 435/287.2

(57) ABSTRACT

The invention pertains to certain genetic variants on Chr2q14, Chr2q35 and Chr16q12 as susceptibility variants of breast cancer. Methods of risk assessment and diagnosis of increased and/or decreased susceptibility to breast cancer, using such variants are described. The invention further relates to kits for diagnosing a susceptibility to breast cancer.

### BACKGROUND OF THE INVENTION

Breast cancer is by far the most common cancer in women worldwide. Current global incidence is in excess of 1,151,000 new cases diagnosed each year (Parkin et al., 2005). Breast cancer incidence is highest in developed countries, particularly amongst populations of Northern European ethnic origin, and is increasing. In the United States the annual age-standardized incidence rate is approximately 131 cases per 100,000 population, more than three times the world average. Rates in Northern European countries are similarly high. In the year 2006 it is estimated that 214,650 new cases of invasive breast cancer will be diagnosed in the U.S.A. and 41,430 people will die from the disease [Jemal, et al., (2006), CA Cancer J Clin, 56, 106-30]. To this figure must be added a further 61,980 ductal and lobular carcinoma in-situ diagnoses expected in 2006. From an individual perspective, the lifetime probability of developing breast cancer is 13.2% in U.S. women (i.e., 1 in 8 women will develop breast cancer during their lives). As with most cancers, early detection and appropriate treatment are important factors. Overall, the 5-year survival rate for breast cancer is 88%. However, in individuals presenting with regionally invasive or metastatic disease, the rate declines to 81% and 26%, respectively [Jemal, et al., (2006), CA Cancer 3 Clin, 56, 106-30].

Increasingly, emphasis is falling on the identification individuals who are at high risk for primary or recurrent breast cancer. Such individuals can be managed by more intensive screening, preventative chemotherapies, hormonal therapies and, in cases of individuals at extremely high risk, prophylactic surgery. Mass screening programs constitute a huge economic burden on health services, while preventative therapies have associated risks and quality of life consequences.

### Genetic Predisposition to Breast Cancer:

The two primary classes of known risk factors for breast cancer are endocrine factors and genetics. Regarding the latter, approximately 12% of breast cancer patients have one or more first degree relatives with breast cancer [(2001), Lancet, 358, 1389-99]. The well known, dominant breast cancer predisposition genes BRCA1 and BRCA2 confer greatly increased breast cancer risk to carriers, with lifetime penetrance estimates ranging from 40-80%. The presence of BRCA1 and BRCA2 mutations can account for the majority of families with 6 or more cases of breast cancer and for a large proportion of families comprising breast and ovarian or male breast cancer. However such families are very rare indeed. BRCA1 and BRCA2 mutations are found much less frequently in families with fewer cases or in families characterised by breast cancer cases only. Together, mutations in BRCA1 and BRCA2 can account for 15-20% of the risk for familial breast cancer. In non-founder populations, if all common BRCA mutations could be detected, between 2-3% of incident breast cancer patients would be expected to harbor a mutation [Gorski, et al., (2005), Breast Cancer Res Treat, 92, 19-24; (2000), Br J Cancer, 83, 1301-8]. This low “chance to find” statistic precludes the responsible use of BRCA mutation testing outside families with an obvious hereditary predisposition (Anon[(2003), J Clin Oncol, 21, 2397-406]). Rare, high penetrance mutations are known to occur in the TP53 and PTEN genes, however, these together account for no more than 5% of the total genetic risk for breast cancer [Easton, (1999), Breast Cancer Res, 1, 14-7]. Linkage studies have been largely unsuccessful in identifying any more, widespread mutations conferring high risk for breast cancer[Smith, et al., (2006), Genes Chromosomes Cancer, 45, 646-55].

Recent epidemiological studies have indicated that the majority of breast cancer cases arise in a predisposed, susceptible minority of the population [Antoniou, et al., (2002), Br J Cancer, 86, 76-83; Pharoah, et al., (2002), Nat Genet, 31, 33-6]. Data from twin studies and observations of the constant, high incidence of cancer in the contralateral breast of patients surviving primary breast cancer indicate that a substantial portion of the uncharacterised risk for breast cancer is related to endogenous factors, most probably genetic [Lichtenstein, et al., (2000), N Engl J Med, 343, 78-85; Peto and Mack, (2000), Nat Genet, 26, 411-4]. Knowledge of the genetic factors that underpin this widespread risk is very limited. Segregation analyses predict that the uncharacterised genetic risk for breast cancer is most likely to be polygenic in nature, with risk alleles that confer low to moderate risk and which may interact with each other and with hormonal risk factors. Nevertheless, these studies predict as much as 40-fold differences in relative risk between the highest and lowest quintiles of a distribution that could be defined by genetic profiling that captures these low to moderate risk alleles [Antoniou, et al., (2002), Br J Cancer, 86, 76-83; Pharoah, et al., (2002), Nat Genet, 31, 33-6]. 88% of all breast cancer cases are expected to arise amongst a predisposed 50% of the population and the 12% of the population at highest risk accounts for 50% of all breast cancer cases [Pharoah, et al., (2002), Nat Genet, 31, 33-6; Pharoah, (2003), Recent Results Cancer Res, 163, 7-18; discussion 264-6]. Much focus is therefore directed towards the identification of such genetically predisposed individuals and developing personalized medical management strategies for them.

We and others have shown that there is a significant familial risk of breast cancer in Iceland which extends to at least 5^{th} degree relatives [Amundadottir, et al., (2004), PLoS Med, 1, e65; Tulinius, et al., (2002), J Med Genet, 39, 457-62]. The contribution of BRCA1 mutations to familial risk in Iceland is thought to be minimal [Arason, et al., (1998), J Med Genet, 35, 446-9; Bergthorsson, et al., (1998), Hum Mutat, Suppl 1, S195-7]. A single founder mutation in the BRCA2 gene (999del5) is present at a carrier frequency of 0.6-0.8% In the general Icelandic population and 7.7-8.6% in female breast cancer patients [Thorlacius, et al., (1997), Am J Hum Genet, 60, 1079-84; Gudmundsson, et al., (1996), Am J Hum Genet, 58, 749-56]. This single mutation is estimated to account for approximately 40% of the inherited breast cancer risk to first through third degree relatives [Tulinius, et al., (2002), J Med Genet, 39, 457-62]. Although this estimate is higher than the 15-25% of familial risk attributed to all BRCA 1 and 2 mutations combined in non-founder populations, there is still some 60% of Icelandic familial breast cancer risk to be explained. First degree relatives of patients who test negative for BRCA2 999del5 remain at a 1.72 fold the population risk for breast cancer (95% CI 1.49-1.96) [Tulinius, et al., (2002), J Med Genet, 39, 457-62].

Understanding of the genetic factors contributing to the residual genetic risk for breast cancer is very limited. Variants in two genes have been rigorously confirmed as low penetrance breast cancer risk genes; CHEK2 and ATM [Renwick, et al., (2006), Nat Genet, 38, 873-5; (2004), Am J Hum Genet, 74, 1175-82]. Many other genes have been implicated however their contribution to breast cancer risk has not been confirmed in analyses employing very large sample sets [Breast Cancer Association, (2006), 3 Natl Cancer Inst, 98, 1382-96].

Genetic risk is conferred by subtle differences in genes among individuals in a population. Genes differ between individuals most frequently due to single nucleotide polymorphisms (SNP), although other variations are also important. SNP are located on average every 1000 base pairs in the human genome. Accordingly, a typical human gene containing 250,000 base pairs may contain 250 different SNP. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphism in the human genome is caused by insertion, deletion, translocation, or inversion of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

No universally successful method for the prevention or treatment of breast cancer is currently available. Management of breast cancer currently relies on a combination of primary prevention, early diagnosis, appropriate treatments and secondary prevention. There are clear clinical imperatives for integrating genetic testing into all aspects of these management areas. Identification of cancer susceptibility genes may also reveal key molecular pathways that may be manipulated (e.g., using small or large molecular weight drugs) and may lead to more effective treatments.

### SUMMARY OF THE INVENTION

The present invention relates to methods of risk assessment of breast cancer. This includes methods of determining an increased susceptibility to breast cancer in an individual, as well as methods of determining a decreased susceptibility to breast cancer or diagnosing a protection against cancer, in an individual, by evaluating certain markers or haplotypes that have been found to be associated with breast cancer, as described further herein.

In a first aspect, the invention pertains to a method for determining a susceptibility to breast cancer in a human individual, comprising determining the presence or absence of at least one allele of at least one polymorphic marker in a nucleic acid sample obtained from the individual or in a genotype dataset derived from the individual, wherein the at least one polymorphic marker is selected from the polymorphic markers set forth in any one of Table 10, Table 15 and Table 19, and markers in linkage disequilibrium therewith, and wherein the presence of the at least one allele is indicative of a susceptibility to breast cancer for the individual. The method may in one embodiment relate to determination of the presence or absence of at least one allele of at least one polymorphic marker in a nucleic acid sample obtained from the individual. In another embodiment, the method relates to determination of the presence or absence of at least one allele of at least one polymorhpic marker in a genotype dataset derived from the individual. The genotype dataset may, in certain embodiments, be derived from a particular individual by virtue of the information contained in the genotype dataset relating to a particular nucleic acid sample template, wherein the nucleic acid sample contains nucleic acid from a single individual.

Determination of the presence of a particular susceptibility allele of a polymorphic marker gives a direct indication of the presence of the particular susceptibility conferred by that allele. Determination of the absence of such a susceptibility allele gives on the other hand indication that the particular susceptibility is absent in the individual from which the genotype sample or gentotype dataset is derived. In the particular case of a polymorphic marker with two possible alleles, such as a SNP or an insertion/deletion polymorphism, the determination of the absence of a particular allele implies the presence of two copies of the alternate allele in the individual (unless the particular genomic region contains a deletion or a duplication in the individual, in which there may be either only a single copy or more than two copies of the particular genomic region in the genome of the individual).

In a second aspect, the present invention relates to a method of diagnosing a susceptibility to breast cancer in a human individual, the method comprising determining the presence or absence of at least one allele of at least one polymorphic marker in a nucleic acid sample obtained from the individual, wherein the at least one polymorphic marker is selected from the group of markers associated with the rs4848543 LD block, the rs3803662 LD block, and the rs13387042 LD block, wherein the presence of the at least one allele is indicative of a susceptibility to breast cancer. In one embodiment, markers associated with the rs4848543 LD block, the rs3803662 LD block and/or the rs13387042 LD block are markers that are in linkage disequilibrium to at least one marker within one or more of these LD blocks.

In a further aspect, the invention relates to a method of diagnosing a susceptibility to breast cancer in a human individual, the method comprising determining the presence or absence of at least one allele of at least one polymorphic marker in a nucleic acid sample obtained from the individual, wherein the at least one polymorphic marker is selected from the group of markers consisting of the markers listed in Table 10, Table 15 and Table 19, and markers in linkage disequilibrium therewith. In one particular embodiment, the markers in linkage disequilibrium with the markers listed in Table 10, Table 15 and Table 19 are those markers with values of r² greater than 0.2. In another embodiment, the markers in linkage disequilibrium with the markers listed in Table 10, Table 15 and Table 19 are those markers with values of r² greater than 0.2 in a Caucasian population, such as the HapMap CEPH population.

In another aspect, the invention pertains to a method of identification of a marker for use in assessing susceptibility to breast cancer, the method comprising:
a. identifying at least one polymorphic marker in linkage disequilibrium with at least one of the markers within the genomic segments with sequence as set forth in SEQ ID NO:4, SEQ ID NO:5 and SEQ ID NO:6;
b. determining the genotype status of a sample of individuals diagnosed with, or having a susceptibility to, breast cancer; and
c. determining the genotype status of a sample of control individuals;
   wherein a significant difference in frequency of at least one allele in at least one polymorphism in individuals diagnosed with, or having a susceptibility to, breast cancer, as compared with the frequency of the at least one allele in the control sample is indicative of the at least one polymorphism being useful for assessing susceptibility to breast cancer. In one embodiment, an increase in frequency of the at least one allele in the at least one polymorphism in individuals diagnosed with, or having a susceptibility to, breast cancer, as compared with the frequency of the at least one allele in the control sample is indicative of the at least one polymorphism being useful for assessing increased susceptibility to breast cancer. In another embodiment, a decrease in frequency of the at least one allele in the at least one polymorphism in individuals diagnosed with, or having a susceptibility to, breast cancer, as compared with the frequency of the at least one allele in the control sample is indicative of the at least one polymorphism being useful for assessing decreased susceptibility to, or protection against, breast cancer. In one embodiment, the at least one polymorphic marker in linkage disequilibrium with at least one of the markers within the genomic segments with sequence as set forth in SEQ ID NO:4, SEQ ID NO:5 and SEQ ID NO:6 is selected from rs4848543 (SEQ ID NO:1), rs3803662 (SEQ ID NO:3) and rs13387042 (SEQ ID NO:2).

The invention furthermore relates to a method of genotyping a nucleic acid sample obtained from a human individual at risk for, or diagnosed with, breast cancer, comprising determining the presence or absence of at least one allele of at least one polymorphic marker in the sample, wherein the at least one marker is selected from the group consisting of the markers set forth in Table 10, Table 15 and Table 19, and markers in linkage disequilibrium therewith, and wherein the presence or absence of the at least one allele of the at least one polymorphic marker is indicative of a susceptibility of breast cancer. In one embodiment, the at least one marker is selected from rs4848543 (SEQ ID NO:1), rs3803662 (SEQ ID NO:3) and rs13387042 (SEQ ID NO:2). In another embodiment, genotyping comprises amplifying a segment of a nucleic acid that comprises the at least one polymorphic marker by Polymerase Chain Reaction (PCR), using a nucleotide primer pair flanking the at least one polymorphic marker. In a further embodiment, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis. In one preferred embodiment, the process comprises allele-specific probe hybridization. In another preferred embodiment, the process comprises DNA sequencing. In a particularly preferred embodiment, the process comprises the steps of
1) contacting copies of the nucleic acid with a detection oligonucleotide probe and an enhancer oligonucleotide probe under conditions for specific hybridization of the oligonucleotide probe with the nucleic acid;
   wherein
   a) the detection oligonucleotide probe is from 5-100 nucleotides in length and specifically hybridizes to a first segment of a nucleic acid whose nucleotide sequence is given by SEQ ID NO:4, SEQ ID NO:5 or SEQ ID NO:6;
   b) the detection oligonucleotide probe comprises a detectable label at its 3′ terminus and a quenching moiety at its 5′ terminus;
   c) the enhancer oligonucleotide is from 5-100 nucleotides in length and is complementary to a second segment of the nucleotide sequence that is 5′ relative to the oligonucleotide probe, such that the enhancer oligonucleotide is located 3° relative to the detection oligonucleotide probe when both oligonucleotides are hybridized to the nucleic acid; and
   d) a single base gap exists between the first segment and the second segment, such that when the oligonucleotide probe and the enhancer oligonucleotide probe are both hybridized to the nucleic acid, a single base gap exists between the oligonucleotides;
2) treating the nucleic acid with an endonuclease that will cleave the detectable label from the 3′ terminus of the detection probe to release free detectable label when the detection probe is hybridized to the nucleic acid; and
   measuring free detectable label, wherein the presence of the free detectable label indicates that the detection probe specifically hybridizes to the first segment of the nucleic acid, and indicates the sequence of the polymorphic site as the complement of the detection probe.

A further aspect of the invention pertains to a method of assessing an individual for probability of response to a breast cancer therapeutic agent, comprising: determining the presence or absence of at least one allele of at least one polymorphic marker in a nucleic acid sample obtained from the individual, wherein the at least one polymorphic marker is selected from the group consisting of the polymorphic markers set forth in Table 10, Table 15 and Table 19, and markers in linkage disequilibrium therewith, wherein the presence of the at least one allele of the at least one marker is indicative of a probability of a positive response to the therapeutic agent. The therapeutic agent is in one embodiment a chemotherapeutic agent or hormonal therapy agent. In one embodiment, the hormonal therapy agent is a Selective Estrogen Receptor Modulator or an Aromatase inhibitor. In preferred embodiments, the Selective Estrogen Receptor Modulator is selected from Tamoxifen and Raloxifene. In certain embodiments, the aromatase inhibitor is selected from Exemestane, Anastrozole and Letrozole. In other embodiments, the Selective Estrogen Receptor Modulator is administered in combination with an aromatase inhibitor.

Yet another aspect of the invention pertains to a method of predicting prognosis of an individual diagnosed with breast cancer, the method comprising determining the presence or absence of at least one allele of at least one polymorphic marker in a nucleic acid sample obtained from the individual, wherein the at least one polymorphic marker is selected from the group consisting of the polymorphic markers set forth in Table 10, Table 15 and Table 19, and markers in linkage disequilibrium therewith, wherein the presence of the at least one allele is indicative of a worse prognosis of the breast cancer in the individual. The prognosis of the individual may in principle relate to any characteristic pattern of progression of the disease, including, but not limited to formation of a secondary tumor, rapid spread of the tumor, the grade of the tumor (Stage 0 through Stage 1V) and the recurrence of the tumor.

The invention also relates to a method of monitoring progress of treatment of an individual undergoing treatment for breast cancer, the method comprising determining the presence or absence of at least one allele of at least one polymorphic marker in a nucleic acid sample obtained from the individual, wherein the at least one polymorphic marker is selected from the markers set forth in Table 10, Table 15 and Table 19, and markers in linkage disequilibrium therewith, wherein the presence of the at least one allele is indicative of the treatment outcome of the individual. Treatment can be any one of surgical treatment, chemotherapy treatment, radiotherapy, gene therapy or immunotherapy. Any of these therapies can be used alone or in combination. For example, surgical treatment may be followed by chemotherapy treatment and/or radiotherapy treatment. The treatment outcome of the individual relates to the tumor progression of the individual at the end of the treatment. Measures of such outcome include, but are not limited to, recurrence rate or likelihood of the tumor, spread of the tumor, and the formation of a secondary tumor.

Another aspect of the invention pertains to a kit for assessing susceptibility to breast cancer in a human individual, the kit comprising reagents for selectively detecting at least one allele of at least one polymorphic marker in the genome of the individual, wherein the polymorphic marker is selected from the markers set forth in Tables 10, 15 and 19, and markers in linkage disequilibrium therewith, and wherein the presence of the at least one allele is indicative of a susceptibility to breast cancer. In one embodiment, the at least one polymorphic marker is rs4848543 (SEQ ID NO:1), rs3803662 (SEQ ID NO:3) or rs13387042 (SEQ ID NO:2). In another embodiment, the reagents comprise at least one contiguous oligonucleotide that hybridizes to a fragment of the genome of the individual comprising the at least one polymorphic marker, a buffer and a detectable label. In another embodiment, the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size. In a further embodiment, the at least one oligonucleotide is completely complementary to the genome of the individual. In another embodiment, the oligonucleotide is from 18 to 50 nucleotides in size. In another embodiment, the oligonucleotide is from 20 to 30 nucleotides in size. In a preferred embodiment, the kit comprises:
a. a detection oligonucleotide probe that is from 5-100 nucleotides in length;
b. an enhancer oligonucleotide probe that is from 5-100 nucleotides in length; and
c. an endonuclease enzyme;
wherein the detection oligonucleotide probe specifically hybridizes to a first segment of the nucleic acid whose nucleotide sequence is set forth in SEQ ID NO:4, SEQ ID NO:5 or SEQ ID NO:6, and
wherein the detection oligonucleotide probe comprises a detectable label at its 3′ terminus and a quenching moiety at its 5′ terminus;
wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid;
wherein a single base gap exists between the first segment and the second segment, such that when the oligonucleotide probe and the enhancer oligonucleotide probe are both hybridized to the nucleic acid, a single base gap exists between the oligonucleotides; and
   wherein treating the nucleic acid with the endonuclease will cleave the detectable label from the 3′ terminus of the detection probe to release free detectable label when the detection probe is hybridized to the nucleic acid.

A further aspect of the invention pertains to an apparatus for determining a genetic indicator for breast cancer in a human individual, comprising:
a computer readable memory; and
a routine stored on the computer readable memory;
   wherein the routine is adapted to be executed on a processor to analyze marker and/or haplotype information for at least one human individual with respect to at least one polymorphic marker selected from the markers set forth in Table 10, Table 15 and Table 19 and markers in linkage disequilibrium therewith, and generate an output based on the marker or haplotype information, wherein the output comprises an individual risk measure of the at least one marker or haplotype as a genetic indicator of breast cancer for the human individual. In one embodiment, the routine further comprises a risk measure for breast cancer associated with the at least one marker allele and/or haplotype, wherein the risk measure is based on a comparison of the frequency of at least one allele of at least one polymorphic marker and/or haplotype in a plurality of individuals diagnosed with breast cancer and an indicator of the frequency of the at least one allele of at least one polymorphic marker and/or haplotype in a plurality of reference individuals, and wherein the individual risk for the human individual is based on a comparison of the carrier status of the individual for the at least one marker allele and/or haplotype and the risk measure for the at least one marker allele and/or haplotype. In one embodiment, the at least one polymorphic marker is selected from rs4848543 (SEQ ID NO:1), rs3803662 (SEQ ID NO:3) or rs13387042 (SEQ ID NO:2), and markers in linkage disequilibrium therewith

The invention also relates to the use of an oligonucleotide probe in the manufacture of a reagent for diagnosing and/or assessing susceptibility to breast cancer in a human individual, wherein the probe hybridizes to a segment of a nucleic acid whose nucleotide sequence is set forth in SEQ ID NO:4, SEQ ID NO:5 or SEQ ID NO:6, wherein the probe is 15-500 nucleotides in length.

The invention further relates to a computer-readable medium on which is stored:
a. an identifier for at least one polymorphic marker;
b. an indicator of the frequency of at least one allele of said at least one polymorphic marker in a plurality of individuals diagnosed with breast cancer; and
c. an indicator of the frequency of the least one allele of said at least one polymorphic markers in a plurality of reference individuals;
   wherein the at least one polymorphic marker is selected from the polymorphic markers set forth in Table 10, Table 15 and Table 19, and polymorphic markers in linkage disequilibrium therewith.

In certain embodiments of the invention, the frequency of at least one haplotype in the individual is further assessed, wherein the haplotype comprises at least two markers, and wherein the presence of the at least one haplotype is indicative of a susceptibility to breast cancer. The haplotype is in one embodiment an at-risk haplotype for breast cancer, i.e., the haplotype confers an increased risk of developing breast cancer. The haplotype is in one embodiment selected from the group of haplotypes listed in Table 7, Table 8, Table 9, Table 13, Table 14 and Table 18.

In another embodiment of the invention, the at least one allele of at least one polymorphic marker is present on a haplotype background selected from the group of haplotype backgrounds defined by the haplotypes listed in Table 9 and Table 13. In another embodiment, the method of the present invention relates to further performing a step of assaying for a high penetrant genetic factor for breast cancer. Such a high penetrant genetic factor is in one embodiment BRCA2 999del5. Recent evidence suggests that risk associated with BRCA1 and BRCA2 variants are in some cases modified by other genetic or environmental factors that cluster in families (Antoniou, A. C., et al. Am J Hum Genet 2008 Mar. 18 (Epub ahead of print)). Thus, in certain embodiments, other high penetrant mutations for breast cancer, such as those found in the BRCA1 and BRCA2 genes, which are well documented and known to the skilled person (see, e.g., Breast Cancer Mutation Data Base at: http://research.nhgri.nih.gov/bic/; see also Fackenthal, J. D. & Olopade, O. I., Nature Reviews Cancer 7:937-48 (2007) and references cited therein) may also be assessed and combined with the variants described herein to be associated with breast cancer.

In one embodiment of the invention, a further step of assessing the individual for estrogen receptor or progesterone receptor status is performed. In one such embodiment, estrogen positive status or progesterone positive status is associated with increased risk associated with rs13387042 allele A and rs3803662 allele T, and markers in association therewith, such as the markers as set forth in Table 15 and Table 19.

The present invention relates, in another aspect, to a method of assessing risk of developing at least a second primary tumor in an individual previously diagnosed with breast cancer, the method comprising determining the presence or absence of at least one allele of at least one polymorphic marker in a nucleic acid sample obtained from the individual, wherein the at least one polymorphic marker is selected from the group of markers associated with the rs4848543 LD Block, the rs13387042 LD block, and the rs3803662 LD block, wherein the presence of the at least one allele is indicative of risk of developing at least a second primary tumor. In one embodiment, the at least one polymorphic marker is selected from the markers set forth in any of the Tables 10, 15 and 19. In another embodiment, the at least one marker is associated with the rs4848543 LD block. In one embodiment, the at least one polymorphic marker is selected from the group consisting of the polymorphic markers listed in Table 10, Table 15, Table 19, Table 20, Table 21 and Table 22, and markers in linkage disequilibrium therewith. In another embodiment, the at least one polymorphic marker is selected from the group of markers in strong linkage disequilibrium, as defined by values of r² greater than 0.2, to marker rs4848543. In yet another embodiment, the at least one polymorphic marker is selected from the group of markers listed in Table7, Table 8 and Table 10. In another embodiment, the at least one polymorphic marker is selected from the markers set forth in Table 10. In a further embodiment, the at least one polymorphic marker is associated with the STEAP3/TSAP6 gene. In a preferred embodiment, marker is rs4848543. In other embodiments, additional steps of assessing the frequency of at least one haplotype in the individual are performed. This at least one haplotype is in one embodiment selected from the group of haplotypes listed in Table 7, Table 8 and Table 9. In other embodiments, high penetrant genetic factors for breast cancer, such as BRCA2 999del5, are also assessed.

The breast cancer phenotype according the invention as described herein can in certain embodiments be selected from All Breast Cancer, Multiple Primary Breast Cancer, early onset Breast Cancer, or other medically accepted diagnostic method of defining Breast Cancer. In another embodiment, the summed family history (FHS) is the phenotype associated with breast cancer.

In particular embodiments of the methods, uses, kits, or apparati of the invention, as described herein, the human individual that is assessed is female.

The markers disclosed herein to be useful for detecting a susceptibility to breast cancer can all be utilized in the various methods, kits, apparati and uses as described herein. Thus, in certain embodiments, the at least one polymorphic marker useful to practice the invention can be selected from the group consisting of the polymorphic markers listed in Tables 20, 21 and 22, and markers in linkage disequilibrium therewith. In another embodiment, the at least one polymorphic marker is selected from the group of markers listed in Table 10, Table 15 and Table 19, and markers in linkage disequilibrium therewith. In certain embodiments, the at least one polymorphic marker is located within the genomic segments with the sequence as set forth in any of SEQ ID NO:4, SEQ ID NO:5 and SEQ ID NO:6. In one preferred embodiment, the at least one marker within SEQ ID NO:4 is selected from the markers set forth in Table 20. In another preferred embodiment, the at least one marker within SEQ ID NO:5 is selected from the markers set forth in Table 21. In yet another preferred embodiment, the at least one marker within SEQ ID NO:6 is selected from the markers set forth in Table 22. In another embodiment, the at least one polymorphic marker is selected from the group of markers listed in Tables 7, 8, 10, 14, 15, 18 and 19. In another embodiment, the at least one polymorphic marker is selected from the group of markers in strong linkage disequilibrium, as defined by values of r² greater than 0.2, to marker rs4848543 (SEQ ID NO:1), rs3803662 (SEQ ID NO:3) or rs13387042 (SEQ ID NO:2). In a preferred embodiment, the at least one polymorphic marker is selected from rs4848543 (SEQ ID NO:1), rs3803662 (SEQ ID NO:3) and rs13387042 (SEQ ID NO:2). In another preferred embodiment, the at least one polymorphic marker is rs4848543 (SEQ ID NO:1). In another preferred embodiment, the at least one polymorphic marker is rs3803662 (SEQ ID NO:3). In yet another preferred embodiment, the at least one polymorphic marker is rs13387042 (SEQ ID NO:2). In a further embodiment, the at least one polymorphic marker is associated with (i.e., in linkage disequilibrium with) the STEAP3/TSAP6 gene, the LOC643714 gene, and/or the TNRC9 gene. In one such embodiment, the at least one polymorphic marker is located within the STEAP3/TSAP6 gene, the LOC643714 gene and/or the TNRC9 gene. In another preferred embodiment, the at least one polymorphic marker is selected from rs4848543, rs13387042, rs3803662, rs12922061, rs4784227 and rs17271951.

In certain methods, uses, kits or apparati of the invention, the presence of at least one allele or haplotype is indicative of increased susceptibility to breast cancer. In other methods of the invention, the presence of at least one allele or haplotype is indicative of decreased susceptibility to breast cancer. Increased susceptibility to breast cancer is in one embodiment present on a haplotype background selected from the group of haplotype backgrounds consisting of
(i) rs8955398 allele 2, rs4848543 allele 1, rs6759589 allele 1, rs838066 allele 2, rs838100 allele 3, rs838086 allele 1, rs12711924 allele 1 and rs3731603 allele 4;
(ii) rs8955398 allele 4, rs4848543 allele 1, rs6759589 allele 1, rs838066 allele 2, rs838100 allele 3, rs838086 allele 1, rs12711924 allele 1 and rs3731603 allele 4;
(iii) rs8955398 allele 4, rs4848543 allele 1, rs6759589 allele 1, rs838066 allele 2, rs838100 allele 3, rs838086 allele 2, rs12711924 allele 1 and rs3731603 allele 4;
(iv) rs8955398 allele 2, rs4848543 allele 1, rs6759589 allele 1, rs838066 allele 2, rs838100 allele 3, rs838086 allele 2, rs12711924 allele 1 and rs3731603 allele 3;
(v) rs8955398 allele 4, rs4848543 allele 1, rs6759589 allele 1, rs838066 allele 2, rs838100 allele 3, rs838086 allele 1, rs12711924 allele 1 and rs3731603 allele 3;
(vi) rs8955398 allele 2, rs4848543 allele 1, rs6759589 allele 1, rs838066 allele 2, rs838100 allele 3, rs838086 allele 1, rs12711924 allele 1 and rs3731603 allele 4.

In another embodiment, increased susceptibility to breast cancer is present on a haplotype background selected from the group of haplotype backgrounds consisting of
(i) rs10191184 allele 3, rs6435957 allele 4, rs10171745 allele 3, rs6716542 allele 1, rs4491709 allele 4, rs12621130 allele 1, rs6735174 allele 1, rs6435959 allele 2, rs2372943 allele 3, rs13387042 allele 1, rs10490444 allele 1, rs13011060 allele 3;
(ii) rs10191184 allele 3, rs6435957 allele 4, rs10171745 allele 3, rs6716542 allele 3, rs4491709 allele 4, rs12621130 allele 1, rs6735174 allele 2, rs6435959 allele 4, rs2372943 allele 3, rs13387042 allele 1, rs10490444 allele 3, rs13011060 allele 3;
(iii) rs10191184 allele 3, rs6435957 allele 2, rs10171745 allele 3, rs6716542 allele 3, rs4491709 allele 4, rs12621130 allele 1, rs6735174 allele 2, rs6435959 allele 4, rs2372943 allele 3, rs13387042 allele 1, rs10490444 allele 3, rs13011060 allele 3;
(iv) rs10191184 allele 3, rs6435957 allele 4, rs10171745 allele 3, rs6716542 allele 3, rs4491709 allele 4, rs12621130 allele 1, rs6735174 allele 1, rs6435959 allele 2, rs2372943 allele 3, rs13387042 allele 1, rs10490444 allele 3, rs13011060 allele 3;
(v) rs10191184 allele 3, rs6435957 allele 4, rs10171745 allele 1, rs6716542 allele 3, rs4491709 allele 2, rs12621130 allele 1, rs6735174 allele 1, rs6435959 allele 2, rs2372943 allele 3, rs13387042 allele 1, rs10490444 allele 1, rs13011060 allele 3;
(vi) rs10191184 allele 3, rs6435957 allele 4, rs10171745 allele 3, rs6716542 allele 3, rs4491709 allele 4, rs12621130 allele 1, rs6735174 allele 2, rs6435959 allele 4, rs2372943 allele 3, rs13387042 allele 1, rs10490444 allele 3, rs13011060 allele 1;
(vii) rs10191184 allele 3, rs6435957 allele 4, rs10171745 allele 3, rs6716542 allele 1, rs4491709 allele 4, rs12621130 allele 1, rs6735174 allele 1, rs6435959 allele 2, rs2372943 allele 3, rs13387042 allele 1, rs10490444 allele 1, rs13011060 allele 1;
(viii) rs10191184 allele 3, rs6435957 allele 2, rs10171745 allele 3, rs6716542 allele 1, rs4491709 allele 4, rs12621130 allele 1, rs6735174 allele 1, rs6435959 allele 2, rs2372943 allele 3, rs13387042 allele 1, rs10490444 allele 1, rs13011060 allele 3;
(ix) rs10191184 allele 3, rs6435957 allele 4, rs10171745 allele 3, rs6716542 allele 1, rs4491709 allele 4, rs12621130 allele 1, rs6735174 allele 1, rs6435959 allele 2, rs2372943 allele 3, rs13387042 allele 1, rs10490444 allele 3, rs13011060 allele 1;
(x) rs10191184 allele 3, rs6435957 allele 2, rs10171745 allele 3, rs6716542 allele 3, rs4491709 allele 4, rs12621130 allele 1, rs6735174 allele 1, rs6435959 allele 2, rs2372943 allele 3, rs13387042 allele 1, rs10490444 allele 1, rs13011060 allele 3.

In a preferred embodiment, the at least one allele or haplotype conferring increased risk of breast cancer is rs4848543 allele 1. In further embodiments, the increased risk is characterized by a relative risk or odds ratio of at least 1.2, including a risk of at least 1.25, a risk of at least 1.3, a risk of at least 1.4, a risk of at least 1.5, a risk of at least 1.6, a risk of at least 1.7, and a risk of at least 2.0.

In certain other embodiments of the invention, the presence of the at least one allele or haplotype is indicative of decreased susceptibility (decreased risk) to breast cancer. The at least one allele or haplotype may for example be selected from the group of marker alleles and haplotypes listed in Tables 8, 9, 13, 14 and 18 having a relative risk (RR) or odds ratio (OR) value of less than one. In one embodiment the at least one allele indicative of decreased susceptibility to breast cancer is present on a haplotype background selected from the group of haplotype backgrounds consisting of
(i) rs895398 allele 2, rs4848543 allele 2, rs6759589 allele 3, rs838066 allele 4, rs838100 allele 1, rs838086 allele 1, rs12711924 allele 3 and rs3731603 allele 3;
(ii) rs895398 allele 2, rs4848543 allele 2, rs6759589 allele 3, rs838066 allele 2, rs838100 allele 1, rs838086 allele 1, rs12711924 allele 3 and rs3731603 allele 3;
(iii) rs895398 allele 2, rs4848543 allele 2, rs6759589 allele 1, rs838066 allele 2, rs838100 allele 3, rs838086 allele 2, rs12711924 allele 3 and rs3731603 allele 3;
(iv) rs895398 allele 2, rs4848543 allele 2, rs6759589 allele 3, rs838066 allele 4, rs838100 allele 3, rs838086 allele 2, rs12711924 allele 3 and rs3731603 allele 3;
(v) rs895398 allele 2, rs4848543 allele 2, rs6759589 allele 3, rs838066 allele 2, rs838100 allele 3, rs838086 allele 2, rs12711924 allele 3 and rs3731603 allele 3;
(vi) rs895398 allele 2, rs4848543 allele 2, rs6759589 allele 1, rs838066 allele 2, rs838100 allele 3, rs838086 allele 1, rs12711924 allele 1 and rs3731603 allele 4;
(vii) rs895398 allele 2, rs4848543 allele 2, rs6759589 allele 3, rs838066 allele 4, rs838100 allele 3, rs838086 allele 1, rs12711924 allele 1 and rs3731603 allele 4;
(viii) rs895398 allele 2, rs4848543 allele 2, rs6759589 allele 1, rs838066 allele 2, rs838100 allele 3, rs838086 allele 1, rs12711924 allele 3 and rs3731603 allele 3; and
(ix) rs895398 allele 2, rs4848543 allele 2, rs6759589 allele 3, rs838066 allele 2, rs838100 allele 1, rs838086 allele 1, rs12711924 allele 1 and rs3731603 allele 4.

In another embodiment, decreased susceptibility to breast cancer is present on a haplotype background selected from the group of haplotype backgrounds consisting of
(i) rs10191184 allele 3, rs6435957 allele 2, rs10171745 allele 3, rs6716542 allele 3, rs4491709 allele 2, rs12621130 allele 3, rs6735174 allele 1, rs6435959 allele 2, rs2372943 allele 3, rs13387042 allele 3, rs10490444 allele 1, rs13011060 allele 3;
(ii) rs10191184 allele 3, rs6435957 allele 4, rs10171745 allele 3, rs6716542 allele 3, rs4491709 allele 4, rs12621130 allele 1, rs6735174 allele 1, rs6435959 allele 4, rs2372943 allele 1, rs13387042 allele 3, rs10490444 allele 3, rs13011060 allele 1;
(iii) rs10191184 allele 1, rs6435957 allele 4, rs10171745 allele 3, rs6716542 allele 3, rs4491709 allele 4, rs12621130 allele 1, rs6735174 allele 1, rs6435959 allele 4, rs2372943 allele 1, rs13387042 allele 3, rs10490444 allele 3, rs13011060 allele 1;
(iv) rs10191184 allele 3, rs6435957 allele 2, rs10171745 allele 3, rs6716542 allele 3, rs4491709 allele 2, rs12621130 allele 3, rs6735174 allele 1, rs6435959 allele 2, rs2372943 allele 3, rs13387042 allele 3, rs10490444 allele 1, rs13011060 allele 1;
(v) rs10191184 allele 1, rs6435957 allele 2, rs10171745 allele 1, rs6716542 allele 3, rs4491709 allele 2, rs12621130 allele 1, rs6735174 allele 1, rs6435959 allele 2, rs2372943 allele 3, rs13387042 allele 3, rs10490444 allele 3, rs13011060 allele 3;
(vi) rs10191184 allele 3, rs6435957 allele 4, rs10171745 allele 3, rs6716542 allele 3, rs4491709 allele 4, rs12621130 allele 1, rs6735174 allele 1, rs6435959 allele 4, rs2372943 allele 1, rs13387042 allele 3, rs10490444 allele 3, rs13011060 allele 3;
(vii) rs10191184 allele 1, rs6435957 allele 2, rs10171745 allele 3, rs6716542 allele 3, rs4491709 allele 4, rs12621130 allele 1, rs6735174 allele 1, rs6435959 allele 4, rs2372943 allele 1, rs13387042 allele 3, rs10490444 allele 3, rs13011060 allele 1;
(viii) rs10191184 allele 3, rs6435957 allele 4, rs10171745 allele 3, rs6716542 allele 3, rs4491709 allele 4, rs12621130 allele 1, rs6735174 allele 1, rs6435959 allele 2, rs2372943 allele 3, rs13387042 allele 3, rs10490444 allele 1, rs13011060 allele 3;
(ix) rs10191184 allele 3, rs6435957 allele 2, rs10171745 allele 3, rs6716542 allele 3, rs4491709 allele 2, rs12621130 allele 1, rs6735174 allele 1, rs6435959 allele 2, rs2372943 allele 3, rs13387042 allele 3, rs10490444 allele 1, rs13011060 allele 3;
(x) rs10191184 allele 1, rs6435957 allele 2, rs10171745 allele 3, rs6716542 allele 3, rs4491709 allele 2, rs12621130 allele 3, rs6735174 allele 1, rs6435959 allele 2, rs2372943 allele 3, rs13387042 allele 3, rs10490444 allele 1, rs13011060 allele 3;
(xi) rs10191184 allele 3, rs6435957 allele 4, rs10171745 allele 3, rs6716542 allele 3, rs4491709 allele 4, rs12621130 allele 1, rs6735174 allele 1, rs6435959 allele 4, rs2372943 allele 3, rs13387042 allele 3, rs10490444 allele 3, rs13011060 allele 1.

In certain embodiments, the decreased susceptibility is characterized by a risk (relative risk or odds ratio) of less than 0.9, including a risk of less than 0.8, a risk of less than 0.7, a risk of less than 0.6, and a risk of less than 0.5.

In principle, assessment of the markers and haplotypes shown herein to be associated with breast cancer can be combined with analysis of a sample comprising genomic DNA from a human individual or a genotype dataset derived from a human individual for the presence or absence of at least one at-risk allele of at least one at-risk variant for breast cancer not in linkage disequilibrium with any one of the markers set forth in Table 10, Table 15 and Table 19. In other words, genetic risk factors not linked to any of the risk factors described herein can be combined with the risk factors of the present invention, so as to obtain an estimate for overall risk for the individual based on a plurality of risk factors. Furthermore, analysis more than one risk factors described herein to be associated with breast cancer can be combined so as to obtain overall combined risk. In one such embodiment, analysis of rs4848543 (SEQ ID NO:1), rs3803662 (SEQ ID NO:3) and rs13387042 (SEQ ID NO:2) is performed for an individual, or a sample from an individual, and overall risk analysis performed. In another embodiment, analysis of rs3803662 (SEQ ID NO:3) and rs13387042 (SEQ ID NO:2) is performed.

The methods, uses, kits and apparati of the invention can in certain embodiments further comprise analyzing non-genetic information to make risk assessment, diagnosis, or prognosis of the individual. Such non-genetic information can in certain embodiments include age, gender, ethnicity, socioeconomic status, previous disease diagnosis, medical history of subject, family history of breast cancer, biochemical measurements, and/or clinical measurements. Combined risk for the genetic and non-genetic risk factors can be performed using methods known to the skilled person.

Linkage disequilibrium (LD) between genetic segments (e.g., markers) is in certain embodiments of the invention characterized by certain values of a linkage disequilibrium measure. As further described herein, linkage disequilibrium can be characterized by particular numerical values of the LD measures r² and |D′|. In one preferred embodiment, linkage disequilibrium is characterized by values for r² of greater than 0.1. In another preferred embodiment, linkage disequilibrium is characterized by values for r² of greater than 0.2. Other cutoff values for r² are also possible, including, but not limited to, 0.3, 0.4, 0.5, 0.6, 0.7, 0.8, 0.9, 0.95, 0.96, 0.97, 0.98, 0.99. In another preferred embodiment, linkage disequilibrium is characterized by values for |D′| of greater than 0.5. In another preferred embodiment, linkage disequilibrium is characterized by values for |D′| of greater than 0.8. Other cutoff values for |D′| are also possible, including, but not limited to, 0.2, 0.3, 0.4, 0.6, 0.7, 0.8, 0.9, 0.95, 0.96, 0.97, 0.98 and 0.99. In certain embodiments, linkage disequilibrium is characterized by numeric cutoff values for either |D′| and r². In one such embodiment linkage disequilibrium is characterized by numeric cutoff values for either |D′| of greater than 0.8 and r² of greater than 0.2, or both. In certain embodiments, LD is determined in a particular population. In certain such embodiments, the population is selected from Caucasian, Chinese, Japanese, and African populations. In one embodiment, the population is the Caucasian CEPH population.

In certain other embodiments of the methods, uses, apparatus or kits of the invention, the individual is of a specific human ancestry. In one embodiment, the ancestry is selected from black African ancestry, Caucasian ancestry and Chinese ancestry. In another embodiment, the ancestry is black African ancestry. In another embodiment, the ancestry is African American ancestry. In another embodiment, the ancestry is European ancestry. In another embodiment, the ancestry is Caucasian ancestry. The ancestry is in certain embodiment self-reported by the individual who undergoes genetic analysis or genotyping. In other embodiments, the ancestry is determined by genetic determination comprising detecting at least one allele of at least one polymorphic marker in a nucleic acid sample from the individual, wherein the presence or absence of the allele is indicative of the ancestry of the individual.

### BRIEF DESCRIPTION OF THE DRAWINGS

The foregoing and other objects, features and advantages of the invention will be apparent from the following more particular description of preferred embodiments of the invention.

FIG. 1 shows the genomic structure of region on chromosome 2 that includes marker rs4848543 that is shown herein to be associated with breast cancer. Markers in linkage disequilibrium with rs4848543 are also found within this region, which is also called rs4848543 LD block herein. The region is characterized by extensive linkage disequilibrium (high LD), and its the boundaries are characterized by regions of high recombination. Markers in linkage disequilibrium with rs4848543, as defined by values for the LD measure r² of greater than 0.2 in the Caucasian CEU HapMap population, span positions 120,023,583-120,117,062 on chromosome 2 in NCBI Build 34, and position 119,644,908-119,738,387 bp in NCBI Build 36. In both of these sequence builds, the region spanned by these markers is 93,479 bp.

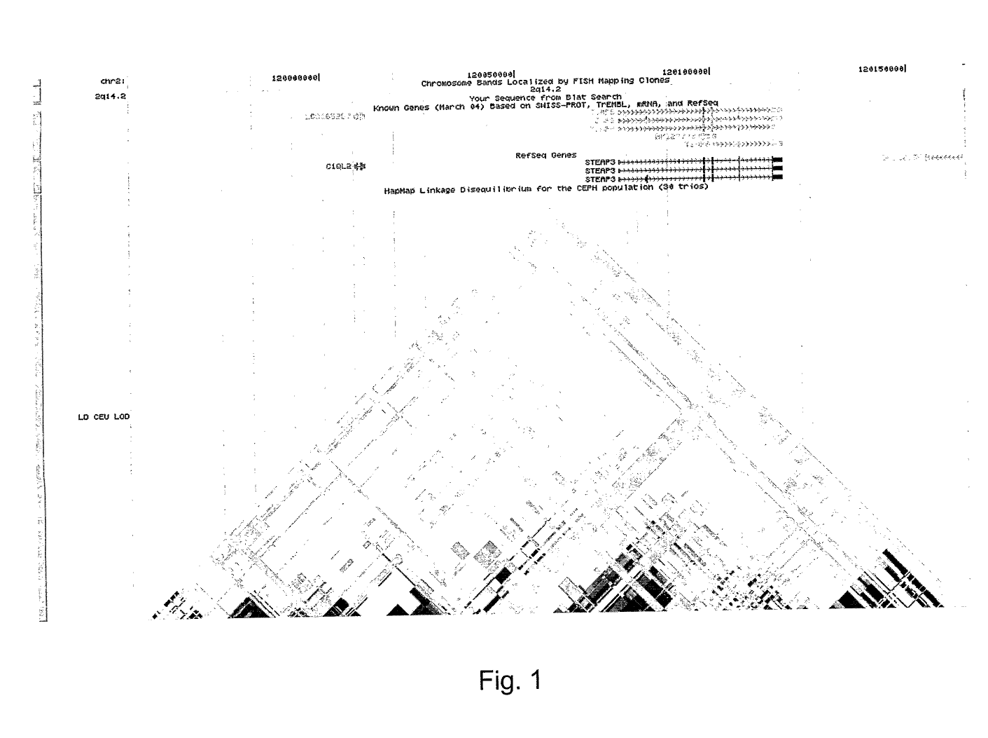

FIG. 2 shows the genomic structure of the region on chromosome 2 that includes marker rs13387042. Markers in linkage disequilibrium with rs13387042 are also found within this region, which is also called rs13387042 LD block herein. The region is characterized by extensive linkage disequilibrium (high LD), and its the boundaries are characterized by regions of high recombination. Markers in linkage disequilibrium with rs13387042, as defined by values for the LD measure r² of greater than 0.2 in the Caucasian CEU HapMap population, span positions 218,059,508-218,141,061 on chromosome 2 in NCBI Build 34, and position 217,565,211-217,646,764 bp in NCBI Build 36. In both of these sequence builds, the region spans 81,553 bp.

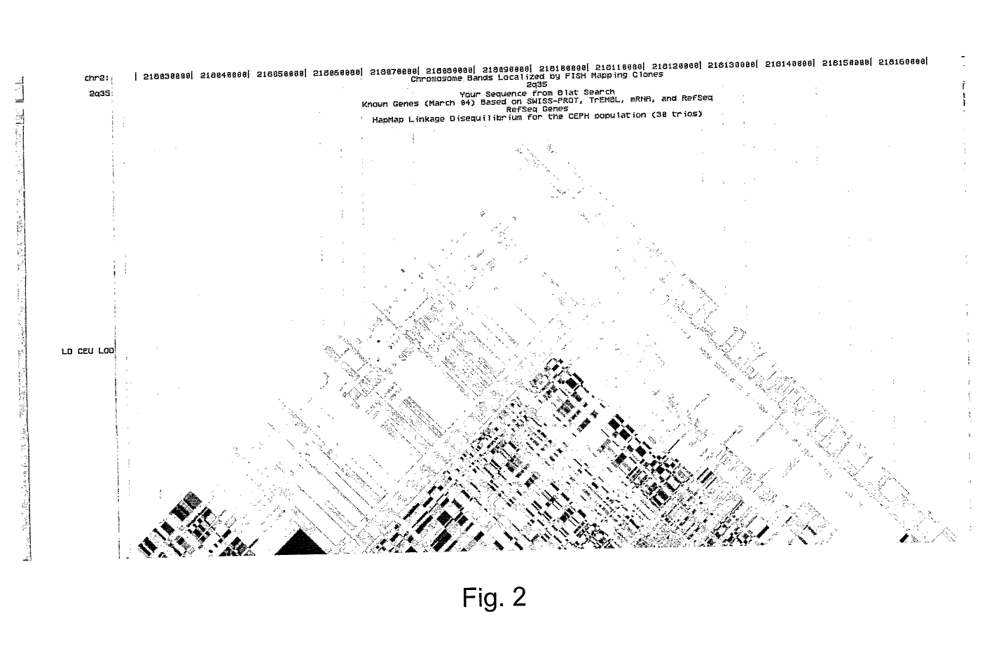

FIG. 3 shows the genomic structure of the region on chromosome 16 that includes marker rs3803662. Markers in linkage disequilibrium with rs3803662 are also found within this region, which is also called rs3803662 LD block herein. The region is characterized by extensive linkage disequilibrium (high LD), and its the boundaries are characterized by regions of high recombination. Markers in linkage disequilibrium with rs3803662, as defined by values for the LD measure r² of greater than 0.2 in the Caucasian CEU HapMap population, span positions 52,314,403-52,413,602 on chromosome 16 in NCBI Build 34, and position 51,093,311-51,192,501 bp in NCBI Build 36. In both of these sequence builds, the region spans 99,190 bp.

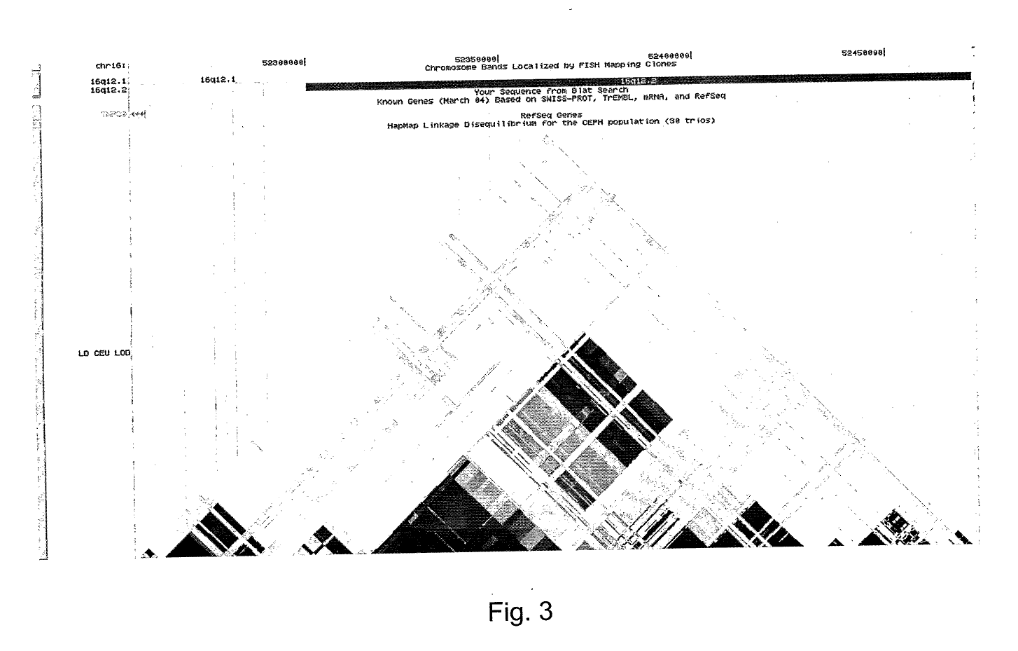

### DETAILED DESCRIPTION OF THE INVENTION

A description of preferred embodiments of the invention follows.

The present invention discloses polymorphic variants and haplotypes that have been found to be associated with breast cancer. Particular alleles at certain polymorphic markers (e.g., marker rs4848543, marker rs13387042 and marker rs3803662, and markers in linkage disequilibrium therewith, the markers associated with the rs4848543 LD block, the rs13387042 LD block, and the rs3803662 LD block, e.g., the markers of Table 10, Table 15, Table 19, Table 20, Table 21 and Table 22, e.g., the markers listed in Table 10, Table 15 and Table 19) and haplotypes comprising such alleles have been found to be associated with breast cancer. Such markers and haplotypes are useful for risk management of breast cancer, as described in further detail herein. Further applications of the invention include kits for assessing the presence or absence of particular alleles of these markers.

### DEFINITIONS

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometimes referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including single nucleotide polymorphisms (SNPs), microsatellites, insertions, deletions, duplications and translocations.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C G or T |
| D | A G or T |
| H | A C or T |
| V | A C or G |
| N | A C G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population.

An “indel” is a common form of polymorphism comprising a small insertion or deletion that is typically only a few nucleotides long.

A “haplotype,” as described herein, refers to a segment of genomic DNA within one strand of DNA that is characterized by a specific combination of alleles arranged along the segment. For diploid organisms such as humans, a haplotype comprises one member of the pair of alleles for each polymorphic marker or locus. In certain embodiments, the haplotype can comprise two or more alleles, three or more alleles, four or more alleles, or five or more alleles. Haplotypes are described herein in the context of the marker name and the allele of the marker in that haplotype, e.g., “1 rs4848543” or “1-rs4848543” refers to the 1 allele (A allele) of marker rs4848543 being in the haplotype, and is equivalent to “rs4848543 allele 1” or “rs4848543 allele A). Furthermore, allelic codes in haplotypes are as for individual markers, i.e. 1=A, 2=C, 3=G and 4=T.

The term “susceptibility”, as described herein, refers to an individual (or group of individuals) being prone to developing breast cancer, or being less able to resist developing breast cancer, than the average individual. The term encompasses both increased susceptibility and decreased susceptibility. Thus, particular polymorphic markers and/or haplotypes of the invention may be characteristic of increased susceptibility (i.e., increased risk) of breast cancer, as characterized by a relative risk (RR) of greater than one or odds ratio (OR) of greater than one. Alternatively, the markers and/or haplotypes of the invention are characteristic of decreased susceptibility (i.e., decreased risk) of breast cancer, as characterized by a relative risk or odds ratio of less than one.

The term “and/or” shall in the present context be understood to indicate that either or both of the items connected by it are involved. In other words, the term herein shall be taken to mean “one or the other or both”.

The term “associated with” shall, in the present context of linking genetic elements (genes and/or markers) refer to the elements being in linkage disequilibrium. Preferably, the term shall be taken to mean that the elements are in linkage disequilibrium as determined by values for the linkage disequilibrium measure r² of greater than 0.2.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” is a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa, placenta, gastrointestinal tract or other organs.

The term “breast cancer therapeutic agent” refers to an agent that can be used to ameliorate or prevent symptoms associated with breast cancer.

The term “breast cancer-associated nucleic acid”, as described herein, refers to a nucleic acid that has been found to be associated to breast cancer. This includes, but is not limited to, the markers and haplotypes described herein and markers and haplotypes in strong linkage disequilibrium (LD) therewith. In one embodiment, a breast cancer-associated nucleic acid refers to a particular genomic region, such as an LD-block, found to be associated with breast cancer through at least one polymorphic marker located within the region or associated with (i.e., in linkage disequilibrium with) the region.

The term “All Breast Cancer”, or “All BC”, refers to all individuals diagnosed with breast cancer, irrespective of the particular subphenotype of the breast cancer.

The term “Medium Predisposition” breast cancer or “MedPre” breast cancer, refers to a sub-phenotype of breast cancer. The definition of this phenotype requires that the proband fulfills at least one of the following criteria:
1. The proband is a member of a cluster of breast cancer cases containing 3 or more affected relatives within a genetic distance of 3 meiotic events (3M).
2. The proband is a member of an affected pair related within 3M, one of whom was diagnosed when aged 50 or younger.
3. The proband is a member of an affected pair related within 3M, one of whom was diagnosed with a second primary tumor of any type.
4. The proband has been diagnosed with a second primary tumor of any type.

Out of the 1600 Icelandic patients analyzed in the study presented herein, 653 fulfills the MedPre criteria (40.8%).

The term “Multiple Primary Breast Tumor”, or “MPBC”, as described herein, refers to cases where at least one Primary tumor is diagnosed in addition to the first breast cancer diagnosis, and the two tumors confirmed both clinically and by histology to be independent primary tumors, arising simultaneously or subsequently to the first breast cancer and occurring in the contralateral or ipsilateral breast.

The term “family history score” or “FHS”, as described herein, is defined based on the number of relatives affected with breast cancer for a proband with the disease. For each proband, a score of 1 is assigned for each affected first-degree relative, 0.5 for each affected second degree relative, and 0.25 for each third-degree relative. The total sum thus obtained over all affected relatives represents the summed family history score or FHS.

The term “rs4848543 LD block”, or “rs4848543 linkage disequilibrium block”, as described herein, refers to the genomic region on Chromosome 2 between positions 119,987,002 and 120,129,001 of NCBI (National Center for Biotechnology Information) Build 34, and between positions 119,608,327 and 120,129,001 in NCBI Build 36. The rs4848543 LD block is 141,999 bp in size.

The term “STEAP3 gene”, or “TSAP6 gene” as described herein, refers to the Six-transmembrane Epithelial Antigen of the Prostate (STEAP3) gene, also called Tumor Suppressor Activated Pathway 6 (TSAP6) gene. The gene is located on chromosome 2q14.2, and spans positions 120076561-120118373 (NCBI Build 34).

The term “rs13387042 LD block”, or “rs13387042 linkage disequilibrium block”, as described herein, refers to the Linkage Disequilibrium (LD) block on Chromosome 2 between positions 218,062,001 and 218,141,002 of NCBI (National Center for Biotechnology Information) Build 34. The LD block spans 79,001 base pairs (Build 34).

The term “rs3803662 LD block”, or “rs3803662 linkage disequilibrium block”, as described herein, refers to the Linkage Disequilibrium (LD) block on Chromosome 16 between positions 52,291,041 and 52,436,127 of NCBI (National Center for Biotechnology Information) Build 34. The LD block spans 145,086 base pairs (Build 34).

The term “Daly haplotype”, as described herein, refers two-marker haplotypes within a given LD block (region of high LD) that are identified as efficient surrogates (r²>0.8) for an additional set of SNPs typed in the HapMap project. These additional SNPs have a minor allele frequency of >5% in the UTAH CEPH (CEU) HapMap samples and they are neither on the Hap300 SNP chip nor are they efficiently tagged by SNPs that are on the chip [Pe'er, et al., (2006), Nat Genet, 38, 663-7].

The term “estrogen receptor positive breast cancer”, or “ER positive breast cancer”, as defined herein, refers to a sample of a breast cancer tissue determined to be positive for expression of estrogen receptor. The expression of estrogen receptor can for example be determined by radioimmunoassay or immunohistochemistry. A radioimmunometric measurement of ≧10 fmol/mg or an immunohistochemical assessment of ≧10% positive nuclei is considered to be positive.

The term “progesterone receptor positive breast cancer”, or “PR positive breast cancer”, as defined herein, refers to a sample of a breast cancer tissue determined to be positive for expression of progesterone receptor. The expression of progestrogen receptor can for example be determined by radioimmunoassay or immunohistochemistry. A radioimmunometric measurement of ≧10 fmol/mg or an immunohistochemical assessment of ≧10% positive nuclei is considered to be positive.

Through association analysis of a population of individuals diagnosed with breast cancer, it has been discovered that certain alleles at certain polymorphic markers are associated with breast cancer. A genome-wide analysis for variants associated with cancer revealed association of breast cancer to two distinct regions of chromosome 2 within the segments Chr2q14 and Chr2q35, as well as within a region on chromosome 16 (Chr16q12). Particular markers and haplotypes were found to be associated with an increased risk of breast cancer in these regions.

As indicated in Table 1, the A allele of marker rs4848543 on chromosome 2q14.2 (also called rs4848543 A allele or rs4848543 1 allele or A-rs4848543) has been found to be associated with an increased risk of breast cancer. Analysis of the MedPre breast cancer phenotype in 1598 patients in relation to 4477 cancer-free population controls, leads to an estimated Relative Risk (RR) value of 1.42, with a p-value of 8.3×10⁻⁸. Following a correction for 317,089 SNP markers tested as part of the genome-wide chip used in the experiment, the p-value is 0.026, i.e. the association is significant at the genome-wide level. The association to the broader All BC phenotype is slightly weaker, with an RR-value of 1.16 (see Table 1). These results have been replicated in an independent Icelandic Breast Cancer cohort.

The rs4848543 marker is located within a region called the rs4848543 LD block herein. This SNP marker, and markers that are correlated with the marker (e.g., markers as listed in Table 10), is useful in the methods of the present invention. Due to the local linkage disequilibrium pattern of the human genome, there are many polymorphic markers that are in strong LD with rs4848543 shown herein to be associated with cancer. These correlated markers (e.g., markers as listed in Table 10), including known SNPs or other polymorphic markers such as microsatellites or indels, as well as other correlated SNPs or other polymorphic markers, could therefore be used, alone or in combination, as surrogate markers for detecting the association to breast cancer described herein. In particular, it is expected that other polymorphic markers located within the rs4848543 LD block, may be used as surrogate markers in the methods of the invention.

A second region on Chromosome 2 (2q35) has been identified through genome-wide association analysis. Allele A of marker rs13387042 (also called rs13387042 A allele or rs13387042 1 allele or A-rs13387042) has been found to be associated with increased risk of breast cancer (see Table 11). This SNP is located at chromosome 2q35, at a distinct location from the STEAP3/TSAP6 locus. An analysis of 2,181 individuals with breast cancer (All BC) and 12,441 population controls reveals that the A allele of rs13387042 confers a risk of 1.19, with a p-value of 4.0×10-5. The risk (RR) in MedPre breast cancer was found to be comparable to that for Any BC. Surrogate markers that could also be used to detect the association to rs13387042 are listed in Table 15.

This result was replicated in a second independent sample from the Icelandic population (583 cases and 7966 controls), giving an estimate for RR for the A allele of rs13387042 in association with AllBC in this second group of 1.20 (P-value_{corr}=3.8×10⁻³). Therefore the original finding was replicated significantly in an independent Icelandic sample, with a very similar point estimate of the RR, and an overall R-value_{corr} 2.0×10⁻⁷, which is close to the level of significance after Bonferroni correction for the 317,089 SNPs tested. The observed frequency and relative risk of the rs13387042 allele A variant corresponds to an estimated population attributable risk of 15.6% in the Icelandic population.

A replication study in Spanish and Swedish samples confirmed these findings. Analysis of 446 breast cancer cases and 977 control samples from Spain gave RR of 1.21 (P-value 1.8×10⁻²; Table 11). The frequency of the A-rs13387042 variant was higher in the Spanish control samples, suggesting that the variant may be more prevalent and thus contribute to a higher Breast Cancer burden in populations of Spanish extraction. Two Swedish cohorts, “Sweden Familial” and “Sweden Consecutive” were also analyzed. The “Sweden Consecutive” cohort demonstrated a significant RR for breast cancer of 1.31 (P-value=2.0×10⁻⁴), while the “Sweden Familial” cohort gave a relative risk estimate of 1.11 that is not statistically significant. Overall, however, the combined Swedish cohorts returned a significant relative risk estimate of 1.22 (P-value=8.1×10⁻⁴).

The estimates for the Icelandic, Spanish and Swedish cohorts were combined in a joint analysis, using the Mantel-Haenszel model. The result was an estimated relative risk of 1.20 with a P-value of 3.8×10⁻¹¹. This is well below the threshold for genome-wide significance using the Bonferroni method to correct for the 317,089 SNPs investigated. We therefore concluded that rs13387042 allele A confers a significant and reproducible risk for breast cancer in several populations samples of European descent, The overall population attributable risk was estimated to be 16.4%.

A further genome-wide SNP analysis using data from 1600 breast cancer patients and 11563 controls identified the T allele of SNP rs3803662 (T-rs3803662; rs3803662 allele T) as conferring an estimated 1.23-fold increased risk for Any BC (Table 16). This result was confirmed in a second, independent cohort of 594 Icelandic breast cancer patients and 1433 controls. The combined data from these two Icelandic samples gave a relative risk estimate of 1.23 and a P-value of 2.8×10⁻⁷ when corrected for relatedness between the individuals. This corresponded to an estimated population attributable risk of 10.1% (Table 16). Surrogate markers that can be used to detect the association to rs3803662 are listed in Table 19.

Replication analysis of this finding in the Swedish and Spanish cohorts described above was performed, as well as an analysis of 558 breast cancer cases and 1384 controls from Nijmegen, Holland. As shown in Table 16, a significantly increased risk for rs3803662 allele T was observed in all three of these replication cohorts. A combined analysis of these three non-Icelandic replication cohorts reveals an overall relative risk estimate of 1.35 and a P-value was 5.1×10⁻¹². A joint analysis of the Icelandic and replication cohorts indicates a combined relative risk estimate of 1.28 with a P-value of 2.7×10⁻¹⁷. This is well below the threshold for genome-wide significance when corrected for the number of SNPs tested. The corresponding overall population attributable risk estimate was 13.4% (Table 16).

The association between rs13387042 allele A and rs3803662 allele T, and estrogen receptor (ER) and progesterone receptor (PR) status was investigated. Significant breast cancer risk involving rs13387042 allele A and rs3803662 allele T was clearly confined to those patients diagnosed with ER positive tumors and the difference between the OR's for ER positive and ER negative tumors was also significant (Table 25). Similarly, there was a tendency towards breast cancer risk preferentially amongst patients diagnosed with PR positive tumors.

### Biology of STEAP3/TSAP6:

A cDNA for STEAP3/TSAP6 was first isolated from one of a series of mRNAs that were induced by p53 activation in the murine myeloid LTR6 cell line, which contains a temperature sensitive p53 gene [Amson, et al., (1996), Proc Natl Acad Sci USA, 93, 3953-7]. This murine form was named Tumour Suppressor Activated Pathway 6 (TSAP6). The gene was later confirmed to be inducible by activated p53 in LTR6 cells [Passer, et al., (2003), Proc Natl Acad Sci USA, 100, 2284-9]. A rat version, designated pHyde, was isolated from a prostatic cancer cell line during experiments designed to detect genes differentially expressed in two cell lines with different metastatic propensities [Rinaldy and Steiner, (1999), DNA Cell Biol, 18, 829-36]. The pHyde cDNA was shown to induce apoptosis in rat and human prostate cell lines and xenografts [Rinaldy, et al., (2000), Gan To Kagaku Ryoho, 27 Suppl 2, 215-22; Steiner, et al., (2000), Cancer Res, 60, 4419-25]. Apoptosis induction by pHyde was later shown to operate through the caspase-3 pathway [Zhang, et al., (2001), Oncogene, 20, 5982-90]. Down regulation of STEAP3/TSAP6 has also been associated with progression of hepatocellular carcinoma [Coulouarn, et al., (2005), J Hepatol, 42, 860-9].

Passer et al. isolated a cDNA for the human version of STEAP3/TSAP6 and showed that the mRNA was inducible by p53 activation in the breast cancer cell line MCF7 [Passer, et al., (2003), Proc Natl Acad Sci USA, 100, 2284-9]. Induction by p53 appears to be at the transcriptional level and is mediated by a conserved p53 response element in the STEAP3/TSAP6 promoter between nucleotides −478 and −357 (sequence defined by accession number AY214461)[Passer, et al., (2003), Proc Natl Acad Sci USA, 100, 2284-9]. The protein was identified as a 488 amino acid, 50-55 kDa, 6-pass transmembrane protein [Passer, et al., (2003), Proc Natl Acad Sci USA, 100, 2284-9]. Antisense RNA targeted on STEAP3/TSAP6 inhibited p53-dependent apoptosis, further supporting the view that STEAP3/TSAP6 acts as an effector of apoptosis [Passer, et al., (2003), Proc Natl Acad Sci USA, 100, 2284-9]. STEAP3/TSAP6 interacts physically with two proteins involved in cell cycle regulation and apoptosis; Nix and Myt1[Passer, et al., (2003), Proc Natl Acad Sci USA, 100, 2284-9]. Nix (also known as BNIP3L) is a mitochondrial, Bcl2-related, proapoptotic protein. Nix and STEAP3/TSAP6 accentuate each other's proapoptotic effects [Passer, et al., (2003), Proc Natl Acad Sci USA, 100, 2284-9]. Myt1 is a dual specificity (Ser/Thr and Tyr) kinase that acts to block the cell cycle at the G2/M checkpoint by phosphorylating and thereby inhibiting the cyclin dependent kinase p34^{cdc2}. The interaction between Myt1 and TSAP6 promotes Myt1-dependent phosphorylation of p34^{cdc2} by holding Myt1 in its hypophosphorylated, active state [Passer, et al., (2003), Proc Natl Acad Sci U S A, 100, 2284-9].

Somewhat paradoxically, STEAP3/TSAP6 was recently identified as the gene responsible for the microcytic, hypochromic anemia in nm1054 mutant stain of mice [Ohgami, et al., (2005), Nat Genet, 37, 1264-9]. Extracellular iron is carried by transferrin (Tf) from the intestine, reticuloendothelial system and liver to all proliferating cells in the body. Tf-bound iron enters the cell by transferrin-receptor (TfR1) mediated endocytosis. Iron is released from Tf in the endosome by acidification, where after it is delivered to the cytoplasm by the divalent metal transporter Dmt1. Tf and TfR1 are then recycled to the extracellular environment. Environmental and Tf-bound iron is present predominantly in the oxidized Fe³⁺ (ferric) state and must be reduced to the Fe²⁺ (ferrous) state before it can be transferred across the cytoplasmic membrane. Through its association with anemia, STEAP3/TSAP6 was identified as the major ferrireductase that carries out this reaction in erythroid lineage cells, implying that it has great importance for iron uptake [Ohgami, et al., (2005), Nat Genet, 37, 1264-9]. STEAP3/TSAP6 also as cupric reductase activity and these metalloreductase activities are shared by other members of the STEAP family STEAP2 and STEAP4 [Ohgami, et al., (2006), Blood, 108, 1388-94].

STEAP3/TSAP6 has also been shown to be involved in stimulation of secretion of proteins via a non-classical pathway. Classical protein secretion is mediated by an NH₂-terminal signal sequence on the protein destined for secretion. The signal sequence directs the protein through the endoplasmic reticulum/Golgi pathway followed by transport the plasma membrane in secretory vesicles. Upon fusion of the secretory vesicle with the plasma membrane, the secreted protein is released into the extracellular space. Protein secretion can also be carried out by a non-classical pathway in which intracellular vesicles can bud out into the lumen of endosomes forming so-called multivesicular structures. Fusion of the multivesicular structure with the plasma membrane results in the release of these membrane-encapsulated vesicles into the extracellular space. These vesicles are known as exosomes. STEAP3/TSAP6 has been shown to stimulate exosome production in a p53 dependent manner [Amzallag, et al., (2004), 7 Biol Chem, 279, 46104-12; Yu, et al., (2006), Cancer Res, 66, 4795-801]. Exosomes may be involved in important anti-oncogenic responses. One of the proteins secreted in exosomes in response to the p53-STEAP3/TSAP6 pathway stimulating is Maspin (mammary serine protease inhibitor) which has proven inhibitory effects on angiogenesis, tumor invasion and metastasis in mammary tumor cells [Sheng, et al., (1996), Proc Natl Acad Sci USA, 93, 11669-74; Shi, et al., (2001), Cancer Res, 61, 6945-51; Zhang, et al., (1997), Mol Med, 3, 49-59; Zou, et al., (1994), Science, 263, 526-9]. Exosomes can contain pro-inflammatory factors such as Translationally Controlled Tumour Protein (TCTP)/Histamine Releasing Factor and tumor-specific antigens such as Her2/Neu and Marti. [Amzallag, et al., (2004), J Biol Chem, 279, 46104-12; Andre, et al., (2002), Lancet, 360, 295-305]. It has been suggested that STEAP3/TSAP6-mediated endosome formation may be responsible for the so-called “bystander” effect wherein cells induced to express p53 can generate cell cycle arrest, proapoptotic responses and death in neighbouring cells [Yu, et al., (2006), Cancer Res, 66, 4795-801].

Almost nothing is known about the relationship between possible genetic variants of STEAP3/TSAP6 and cancer risk. A screen for mutations in prostate cancer cell lines, xenografts and tumor samples found 2 missense mutations (Ala184Thr and Ile305Thr) in xenografts out of a total of 4 cell lines, 8 xenografts and 56 tumor samples (total 68 samples). The authors concluded that STEAP3/TSAP6 is not a classical tumor suppressor gene in prostate cancer. No studies of STEAP3/TSAP6 genetic variants in any other type of cancer have been reported to our knowledge.

### Potential for Utilization of the STEAP3/TSAP6 Pathway in the Development of Improved Therapies for Breast Cancer

Our observation that a genetic variant in the STEAP3/TSAP6 LD block implicates this gene as a common factor in the development of breast carcinoma. Therefore therapies which target STEAP3/TSAP6, its homologues and other components on the STEAP3/TSAP6 pathways can be considered as candidates for the treatment or prevention of breast cancer. Such targets include: STEAP3/TSAP6 itself (OMIM#609671); the homologues STEAP1 (OMIM#604415), STEAP2/TIARP/STAMP1 (OMIM#605094), and STEAP4 (NM_{—}024636); interacting proteins Nix/BNIP3L (OMIM#605368), Myt1 (OMIM#602474), TCTP/Histamine Releasing Factor (OMIM#600763); pathway proteins p34^{cdc2} (OMIM#116940), cyclin B1 (OMIM#123836), HER2/Neu (OMIM#164870), Maspin (OMIM#154790); and other members of the STEP3/TSAP6 pathway yet to be identified or implicated. Therapies could include small molecule agonists or antagonists of STEAP3/TSAP6, its homologues or pathway components; macromolecular agents as agonists or antagonists of STEAP3/TSAP6, its homologues or pathway components; or the use of STEAP3/TSAP6, homologues or pathway components as transgenes in gene therapy or immunotherapy protocols. STEAP3/TSAP6, its homologues or pathway components and/or molecules that affect their activities could also be developed as adjuvants for cytostatic or cytotoxic therapies (chemotherapy, radiotherapy, gene or immunotherapy) through their potentiation of the “bystander effect”.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. All sequence variants can be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realize that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+strand or −strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Additional variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific methodologies available for SNP genotyping include, but are not limited to, TaqMan genotyping assays and SNPlex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), and BeadArray Technologies (e.g., Illumina GoldenGate and Infinium assays). By these or other methods available to the person skilled in the art, one or more alleles at polymorphic markers, including microsatellites, SNPs or other types of polymorphic markers, can be identified.

In certain methods described herein, an individual who is at an increased susceptibility (i.e., increased risk) for any specific disease or trait under study (e.g., breast cancer), is an individual in whom at least one specific allele at one or more polymorphic marker or haplotype conferring increased susceptibility for the disease or trait is identified (i.e., at-risk marker alleles or haplotypes). In one aspect, the at-risk marker or haplotype is one that confers a significant increased risk (or susceptibility) of breast cancer. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, at least 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least about 1.5 is significant. In another further embodiment, a significant increase in risk is at least about 1.7 is significant. However, other cutoffs are also contemplated, e.g. at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype of the present invention is one where at least one allele of at least one marker or haplotype is more frequently present in an individual at risk for breast cancer (affected), compared to the frequency of its presence in a comparison group (control), and wherein the presence of the marker or haplotype is indicative of susceptibility to the disease or trait. The control group may in one embodiment be a population sample, i.e. a random sample from the general population. In another embodiment, the control group is represented by a group of individuals who are disease-free, e.g., individuals who have not been diagnosed with breast cancer. Such disease-free control may in one embodiment be characterized by the absence of one or more specific disease-associated symptoms. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors are at least one genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

In other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied, and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

A genetic variant associated with a disease or a trait (e.g. breast cancer) can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, J., et al., Nat Genet 39:631-7 (2007), Gudmundsson, J., et al., Nat Genet 39:977-83 (2007); Yeager, M., et al, Nat Genet 39:645-49 (2007), Amundadottir, L., el al., Nat Genet 38:652-8 (2006); Haiman, C. A., et al., Nat Genet 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with a plurality of variants associated with breast cancer may be assessed. In one such embodiment, the markers rs4848543, rs13387042 and rs3803662, or surrogate markers in linkage disequilibrium with these markers, are assessed and the risk for the genotype combinations (3³=27 possible combinations) calculated. In another embodiment, genotypes for two of the markers (e.g., markers rs13387042 and rs3803662) are combined to give overall risk. In other embodiments, additional markers known to predispose to breast cancer (e.g., high penetrant risk factors, such as BRCA1, BRCA2, BARD1) are combined with one or more of the risk factors described herein.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275 (2002)).

Linkage Disequilibrium (LD) refers to a non-random assortment of two genetic elements. For example, if a particular genetic element (e.g., “alleles” of a polymorphic marker) occurs in a population at a frequency of 0.50 (50%) and another occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium since they tend to be inherited together at a higher rate than what their independent allele frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and/or |D′|>0.8. In another embodiment, a significant linkage disequilibrium is defined as r²>0.2 and/or |D′|>0.8. In another embodiment, a significant linkage disequilibrium is defined as r²>0.2 and/or |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian, african, Japanese, chinese), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were identical at the population level, then every single one of them would need to be investigated in association studies. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Some representative methods for identification of haplotype blocks are set forth, for example, in U.S. Published Patent Application Nos. 20030099964, 20030170665, 20040023237 and 20040146870. Haplotype blocks can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It is well known that linkage disequilibrium may vary between populations, due to differential historical rates of assortment in the various populations. In certain embodiments of the present invention, LD refers to LD as determined in Caucasian samples. In a particular embodiment, LD is determined in Caucasian CEPH samples from the HapMap consortium (as described on http://www.hapmap.org). In other embodiments, LD is determined in African populations, African-American populations, Hispanic populations, Japanese populations, Chinese populations. In certain embodiments, LD is determined in the HapMap samples from China, Japan, Africa, as described (http://www.hapmap.org).

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait. One or more causative (functional) variants or mutations may reside within the region found to be associating to the disease or trait. Such variants may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a linkage region, for example, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of an significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Linkage Disequilibrium Using NEMO

LD between pairs of markers can be calculated using the standard definition of D′ and r² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and r² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The Risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

As described herein, certain polymorphic markers and haplotypes comprising such markers are found to be useful for risk assessment of breast cancer. Risk assessment can involve the use of the markers for diagnosing a susceptibility to breast cancer. Particular alleles of polymorphic markers are found more frequently in individuals with breast cancer, than in individuals without diagnosis of breast cancer. Therefore, these marker alleles have predictive value for detecting breast cancer, or a susceptibility to breast cancer, in an individual. Tagging markers within haplotype blocks or LD blocks comprising at-risk markers, such as the markers of the present invention, can be used as surrogates for other markers and/or haplotypes within the haplotype block or LD block. Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high or possibly even higher than the at-risk variant. The at-risk variant identified may not be the functional variant itself, but is in this instance in linkage disequilibrium with the true functional variant. The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, within the haplotype or LD block, also have predictive value for detecting association to breast cancer, or a susceptibility to breast cancer, in an individual. These tagging or surrogate markers that are in LD with the markers of the present invention can also include other markers that distinguish among haplotypes, as these similarly have predictive value for detecting susceptibility to breast cancer.

The present invention can in certain embodiments be practiced by assessing a sample comprising genomic DNA from an individual for the presence of variants described herein to be associated with breast cancer. Such assessment includes steps of detecting the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of breast cancer. Alternatively, the invention can be practiced utilizing a dataset comprising information about the genotype status of at least one polymorphic marker described herein to be associated with breast cancer (or markers in linkage disequilibrium with at least one marker shown herein to be associated with breast cancer). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with breast cancer. A positive result for a variant (e.g., marker allele) associated with breast cancer, as shown herein, is indicative of the individual from which the dataset is derived is at increased susceptibility (increased risk) of breast cancer.

In certain embodiments of the invention, a polymorphic marker is correlated to breast cancer by referencing genotype data for the polymorphic marker to a look-up table that comprises correlations between at least one allele of the polymorphism and breast cancer. In some embodiments, the table comprises a correlation for one polymorphism. In other embodiments, the table comprises a correlation for a plurality of polymorphisms. In both scenarios, by referencing to a look-up table that gives an indication of a correlation between a marker and breast cancer, a risk for breast cancer, or a susceptibility to breast cancer, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers and haplotypes of the invention, e.g., the polymorphic markers and haplotypes on chromosome 2q14.2, chromosome 2q35, and chromosome 16q12, e.g., the markers presented in Table 10, Table 15, Table 19, Table 20, Table 21 and Table 22, and markers in linkage disequilibrium therewith, e.g., markers rs4848543, rs13387042 and rs3803662, may be useful for risk assessment and diagnostic purposes for, either alone or in combination. Thus, even in cases where the increase in risk by individual markers is relatively modest, i.e. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in one embodiment of the invention, a plurality of variants (markers and/or haplotypes) is used for overall risk assessment. These variants are in one embodiment selected from the variants as disclosed herein. Other embodiments include the use of the variants of the present invention in combination with other variants known to be useful for diagnosing a susceptibility to breast cancer. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, may subsequently be used to determine the overall risk conferred based on the genotype status at the multiple loci. Assessment of risk based on such analysis may subsequently be used in the methods and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined. Such markers and/or haplotypes may in those cases be also used as surrogate markers and/or haplotypes for the markers and/or haplotypes physically residing within the haplotype block as defined. As a consequence, markers and haplotypes in LD (typically characterized by r² greater than 0.1, such as r² greater than 0.2, including r² greater than 0.3, also including r² greater than 0.4) with the markers and haplotypes of the present invention are also within the scope of the invention, even if they are physically located beyond the boundaries of the haplotype block as defined. This includes markers that are described herein (e.g., Table 10, Table 15 and Table 19), but may also include other markers that are in strong LD (e.g., characterized by r² greater than 0.1, r² greater than 0.2 and/or 1>0.8) with one or more of the markers listed in Table 10, Table 15 and Table 19.

For the SNP markers described herein, the opposite allele to the allele found to be in excess in patients (at-risk allele) is found in decreased frequency in breast cancer. These markers and haplotypes in LD and/or comprising such markers, are thus protective for breast cancer, i.e. they confer a decreased risk or susceptibility of individuals carrying these markers and/or haplotypes developing breast cancer.

Certain variants of the present invention, including certain haplotypes comprise, in some cases, a combination of various genetic markers, e.g., SNPs and microsatellites. Therefore, detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with breast cancer, (e.g., marker alleles as listed in Table 10, Table 15, Table 19, Table 20, Table 21 and Table 22, and markers in linkage disequilibrium therewith) is one in which the marker allele or haplotype is more frequently present in an individual at risk for breast cancer (affected), compared to the frequency of its presence in a healthy individual (control), wherein the presence of the marker allele or haplotype is indicative of breast cancer or a susceptibility to breast cancer. In other embodiments, at-risk markers in linkage disequilibrium with one or more markers found to be associated with breast cancer (e.g., markers as listed in Table 10, Table 15, Table 19, Table 20, Table 21 and Table 22, and markers in linkage disequilibrium therewith) are tagging markers that are more frequently present in an individual at risk for breast cancer (affected), compared to the frequency of their presence in a healthy individual (control), wherein the presence of the tagging markers is indicative of increased susceptibility to breast cancer. In a further embodiment, at-risk markers alleles (i.e. conferring increased susceptibility) in linkage disequilibrium with one or more markers found to be associated with breast cancer (e.g., marker alleles as listed in Table 10, Table 15, Table 19, Table 20, Table 21 and Table 22, and markers in linkage disequilibrium therewith), are markers comprising one or more allele that is more frequently present in an individual at risk for breast cancer, compared to the frequency of their presence in a healthy individual (control), wherein the presence of the markers is indicative of increased susceptibility to breast cancer.

### Study Population

In a general sense, the methods and kits of the invention can be utilized from samples containing genomic DNA from any source, i.e. any individual. In preferred embodiments, the individual is a human individual. The individual can be an adult, child, or fetus. The present invention also provides for assessing markers and/or haplotypes in individuals who are members of a target population. Such a target population is in one embodiment a population or group of individuals at risk of developing the disease, based on other genetic factors, biomarkers, biophysical parameters (e.g., weight, BMD, blood pressure), or general health and/or lifestyle parameters (e.g., history of disease or related diseases, previous diagnosis of disease, family history of disease).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of the disease in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either sex, males or females. In one embodiment, it relates to assessment of male subjects. In another embodiment, it relates to assessment of female subjects.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Stacey, S. N., et al., Nat Genet. May 27, 2007 (Epub ahead of print; Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Amundadottir, et al., Nat Genet, 38:652-58 (2006); Grant, S. F., et al., Nat Genet. 38:320-23 (2006)). Thus, genetic findings in the Icelandic population have in general been replicated in other populations, including populations from Africa and Asia.

The markers of the present invention found to be associated with breast cancer are believed to show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portugues, Italian, Polish, Bulgarian, Slavic, Serbian, Bosnian, Chech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Orogen, She, Tujia, Tu, Xibo, Yi, Mongolan, Naxi, Cambodian, Japanese, Yakut, Melanesian, Papuan, Karitianan, Surui, Colombian, Maya and Pima.

In one embodiment, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry.

The racial contribution in individual subjects may also be determined by genetic analysis. Genetic analysis of ancestry may be carried out using unlinked microsatellite markers such as those set out in Smith et al. Am J Hum Genet 74, 1001-13 (2004).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the variants described herein in general do not, by themselves, provide an absolute identification of individuals who will develop breast cancer. The variants described herein do however indicate increased and/or decreased likelihood that individuals carrying the at-risk or protective variants of the invention will develop breast cancer. This information is however extremely valuable in itself, as outlined in more detail in the below, as it can be used for disease management and selection of appropriate treatment options.

The knowledge about a genetic variant that confers a risk of developing a disease (e.g., breast cancer) offers the opportunity to apply a genetic test to distinguish between individuals with increased risk of developing the disease (i.e. carriers of the at-risk variant) and those with decreased risk of developing the disease (i.e. carriers of the protective variant). The core values of genetic testing, for individuals belonging to both of the above mentioned groups, are the possibilities of being able to diagnose the disease, or a predisposition to the disease, at an early stage and provide information to the clinician about prognosis/aggressiveness of disease in order to be able to apply the most appropriate treatment.

### Models to Predict Inherited Risk for Breast Cancer

The goal of breast cancer risk assessment is to provide a rational framework for the development of personalized medical management strategies for all women with the aim of increasing survival and quality of life in high-risk women while minimizing costs, unnecessary interventions and anxiety in women at lower risk. Risk prediction models attempt to estimate the risk for breast cancer in an individual who has a given set of congenital risk characteristics (e.g., family history, prior benign breast lesion, previous breast tumor). The breast cancer risk assessment models most commonly employed in clinical practice estimate inherited risk factors by considering family history. The risk estimates are based on the observations of increased risks to individuals with one or more close relatives previously diagnosed with breast cancer. They do not take into account complex pedigree structures. These models have the further disadvantage of not being able to differentiate between carriers and non-carriers of genes with breast cancer predisposing mutations.

More sophisticated risk models have better mechanisms to deal with specific family histories and have an ability to take into account carrier status for BRCA1 and BRCA2 mutations. For example, the Breast and Ovarian Analysis of Disease Incidence and Carrier Estimation Algorithm (BOADICEA) (Antoniou et al., 2004) takes into account family history based on individual pedigree structures through the pedigree analysis program MENDEL. Information on known BRCA1 and BRCA2 status is also taken into account. The main limitations of the BOADICEA and all other breast cancer risk models currently in use are that they do not incorporate genotypic information from other predisposition genes. Current models depend strongly on family history to act as a surrogate to compensate for the lack of knowledge of non-BRCA genetic determinants of risk. Therefore the available models are limited to situations where there is a known family history of disease. Lower penetrance breast cancer predisposition genes may be relatively common in the population and may not show such strong tendencies to drive familial clustering as do the BRCA1 and BRCA2 genes. Patients with a relatively high genetic load of predisposition alleles may show little or no family history of disease. There is a need therefore to construct models which incorporate inherited susceptibility data obtained directly through gene-based testing. In addition to making the models more precise, this will reduce the dependency on family history parameters and assist in the extension of the risk profiling into the wider at-risk population where family history is not such a key factor.

Integration of Improved Genetic Risk Models into Clinical Management of Breast Cancer Primary Prevention

Clinical primary prevention options currently can be classified as chemopreventative (or hormonal) treatments and prophylactic surgery. Patients identified as high risk can be prescribed long-term courses of chemopreventative therapies. This concept is well accepted in the field of cardiovascular medicine, but is only now beginning to make an impact in clinical oncology. The most widely used oncology chemopreventative is Tamoxifen, a Selective Estrogen Receptor Modulator (SERM). Initially used as an adjuvant therapy directed against breast cancer recurrence, Tamoxifen now has proven efficacy as a breast cancer preventative agent (Cuzick et al., 2003; Martino et al., 2004). The FDA has approved the use of Tamoxifen as a chemopreventative agent in certain high risk women.

Unfortunately, long term Tamoxifen use increases risks for endometrial cancer approximately 2.5-fold, the risk of venous thrombosis approximately 2.0-fold. Risks for pulmonary embolism, stroke, and cataracts are also increased (Cuzick et al., 2003). Accordingly, the benefits in Tamoxifen use for reducing breast cancer incidence may not be easily translated into corresponding decreases in overall mortality. Another SERM called Raloxifene may be more efficacious in a preventative mode, and does not carry the same risks for endometrial cancer. However risk for thrombosis is still elevated in patients treated long-term with Raloxifene (Cuzick et al., 2003; Martino et al., 2004). Moreover, both Tamoxifen and Raloxifene have quality of life issues associated with them. To make a rational risk:benefit analysis of SERM therapy in a chemopreventative mode, there is a clinical need to identify individuals who are most at risk for breast cancer. Given that a substantial proportion of risk for breast cancer is genetic, there is a clear clinical need for genetic tests to quantify individuals' risks in this context. One can anticipate similar issues arising from any future cancer chemo-preventative therapies that may become available, such as the aromatase inhibitors. Moreover, as chemopreventative therapies become safer, there is an increased need to identify patients who are genetically predisposed, but do not have massively elevated risks associated with BRCA1 & 2 carriers.

Patients who are identified as being at high risk for breast cancer are considered for prophylactic surgery; either bilateral mastectomy or oophorectomy or both. Clearly such drastic treatments are recommended only for patients who are perceived to be at extremely high risk. In practice, such risks can currently be identified only in individuals who carry mutations in BRCA1, BRCA2 or genes known to be involved in rare breast cancer predisposition syndromes like p53 in Li-Fraumeni Syndrome, PTEN in Cowden's Syndrome.

Estimates of the penetrance of BRCA1 and BRCA2 mutations tend to be higher when they are derived from multiple-case families than when they are derived from population-based estimates. This is because different mutation-carrying families exhibit different penetrances for breast cancer (see Thorlacius et al., 1997, for example). One of the major factors contributing to this variation is the action of as yet unknown predisposition genes whose effects modify the penetrance of BRCA1 and BRCA2 mutations. Therefore the absolute risk to an individual who carries a mutation in the BRCA1 or BRCA2 genes cannot be accurately quantified in the absence of knowledge of the existence and action of modifying genes. Since the treatment options for BRCA1 and BRCA2 carriers can be severe, it is important in this context to quantify the risks to individual BRCA carriers with the greatest accuracy possible. There is a need, therefore, to identify predisposition genes whose effects modify the penetrance of breast cancer in BRCA1 and BRCA2 carriers and to develop improved risk assessment models based on these genes.

Furthermore, there are individuals who are perceived to be at very high risk for breast cancer, perhaps because of a strong family history of breast cancer, but in whom no mutations in known predisposition genes can be identified. Consideration of prophylactic surgery is difficult in such cases because one cannot test the individual to discover whether or not she has inherited a high penetrance predisposition gene. Accordingly, the individual's risk cannot be assessed accurately. There is a clear clinical need, therefore, to identify any high penetrance predisposition genes that remain undiscovered and to develop associated genetic tests for use primary prevention strategies.

### Early Diagnosis

Clinical screening for breast cancer in most western countries consists of periodic clinical breast examination (CBE) and X-ray mammography. There is good evidence to indicate that CBE has little added benefit when used in the context of a good mammographic screening program. In the United Kingdom, women between the ages of 50 and 70 are invited to undergo screening mammography every three years. The situation in the United States varies depending on healthcare provider, however the American Cancer Society recommends annual mammographic screening from age 40. Mammographic screening has proven effectiveness in reducing mortality amongst screened women over the age of 50.

It is unlikely that genetic testing would ever be employed as a means of reducing access to existing mammographic screening programs. However, mammographic screening is not without shortcomings and it is conceivable that genetic testing should be used to select people for augmented screening programs. One of the drawbacks of mammographic screening is that is has thus far not been possible to demonstrate a significant effect on improved survival for women screened under 50 years of age.

One reason that mammography is less effective in women under 50 may be that the density of breast tissue is higher in younger women, making mammographic detection of tumors more difficult. However, breast cancers in predisposed individuals tend to occur at early ages groups and there is a clear association between high breast density and breast cancer risk. Therefore there is a problem with simple increases in mammographic screening for individuals with high predisposition because they would be managed by a technique that performs sub-optimally in the group at highest risk. Recent studies have shown that contrast-enhanced magnetic resonance imaging (CE-MRI) is more sensitive and detects tumors at an earlier stage in this high-risk group than mammographic screening does (Warner et al., 2004; Leach et al., 2005). CE-MRI strategies work particularly well when used in combination with routine X-ray mammography (Leach et al., 2005). Because CE-MRI requires specialist centers that incur high costs, screening of under-50's must be restricted to those individuals at the highest risk. Present CE-MRI trials restrict entry to those individuals with BRCA1, BRCA2 or p53 mutations or very strong family histories of disease. The extension of this screening modality to a wider range of high-risk patients would be greatly assisted by the provision of gene-based risk profiling tools.

There is good evidence to support the notion that early-onset breast cancers and cancers occurring in genetically predisposed women grow faster than cancers in older, less strongly predisposed women. This comes from observations of higher rates of interval cancers in younger women, that is, cancers that arise in the intervals between screening visits in a well-screened population are higher amongst younger women. Therefore there are suggestions that screening intervals, by whatever method, should be reduced for younger women. There is a paradox here in that more frequent screening using more expensive methodologies seems to be required for an age group in which the overall rates of breast cancer are comparatively low. There is a clear clinical need here to identify those young individuals who are most strongly predisposed to develop the disease early, and channel them into more expensive and extensive screening regimes.

### Treatment

Currently, primary breast cancer is treated by surgery, adjuvant chemotherapy, radiotherapy, followed by long term hormonal therapy. Often combinations of three or four therapies are used.

Breast cancer patients with the same stage of disease can have very different responses to adjuvant chemotherapy resulting in a broad variation in overall treatment outcomes. Consensus guidelines (the St Galen and NIH criteria) have been developed for determining the eligibility of breast cancer patients for adjuvant chemotherapy treatment. However, even the strongest clinical and histological predictors of metastasis fail to predict accurately the clinical responses of breast tumors (Goldhirsch et al., 1998; Eifel et al., 2001). Chemotherapy or hormonal therapy reduces the risk of metastasis only by approximately ⅓, however 70-80% of patients receiving this treatment would have survived without it. Therefore the majority of breast cancer patients are currently offered treatment that is either ineffective or unnecessary. There is a clear clinical need for improvements in the development of prognostic measures which will allow clinicians to tailor treatments more appropriately to those who will best benefit. It is reasonable to expect that profiling individuals for genetic predisposition may reveal information relevant to their treatment outcome and thereby aid in rational treatment planning.

Several previous studies exemplify this concept: Breast cancer patients who are BRCA mutation carriers appear to show better clinical response rates and survival when treated with adjuvant chemotherapies [Chappuis, et al., (2002), J Med Genet, 39, 608-10; Goffin, et al., (2003), Cancer, 97, 527-36]. BRCA mutation carriers demonstrate improved responses to platinum chemotherapy for ovarian cancer than non-carriers [Cass, et al., (2003), Cancer, 97, 2187-95]. Similar considerations may apply to predisposed patients in whom the genes involved are not known. For example, infiltrating lobular breast carcinoma (ILBC) is known to have a strong familial component but the genetic variants involved have not yet been identified. Patients with ILBC demonstrate poorer responses to common chemotherapy regimes [Mathieu, et al., (2004), Eur Cancer, 40, 342-51].

Genetic predisposition models may not only aid in the individualization of treatment strategies, but may play an integral role in the design of these strategies. For example, BRCA1 and BRCA2 mutant tumor cells have been found to be profoundly sensitive to poly (ADP-ribose) polymerase (PARP) inhibitors as a result of their defective DNA repair pathway [Farmer, et al., (2005), Nature, 434, 917-21]. This has stimulated development of small molecule drugs targeted on PARP with a view to their use specifically in BRCA carrier patients. From this example it is clear that knowledge of genetic predisposition may identify drug targets that lead to the development of personalized chemotherapy regimes to be used in combination with genetic risk profiling.

Cancer chemotherapy has well known, dose-limiting side effects on normal tissues particularly the highly proliferate hemopoetic and gut epithelial cell compartments. It can be anticipated that genetically-based individual differences exist in sensitivities of normal tissues to cytotoxic drugs. An understanding of these factors might aid in rational treatment planning and in the development of drugs designed to protect normal tissues from the adverse effects of chemotherapy.

Genetic profiling may also contribute to improved radiotherapy approaches: Within groups of breast cancer patients undergoing standard radiotherapy regimes, a proportion of patients will experience adverse reactions to doses of radiation that are normally tolerated. Acute reactions include erythema, moist desquamation, edema and radiation pneumatitis. Long term reactions including telangiectasia, edema, pulmonary fibrosis and breast fibrosis may arise many years after radiotherapy. Both acute and long-term reactions are considerable sources of morbidity and can be fatal. In one study, 87% of patients were found to have some adverse side effects to radiotherapy while 11% had serious adverse reactions (LENT/SOMA Grade 3-4); [Hoeller, et al., (2003), Int J Radiat Oncol Biol Phys, 55, 1013-8]. The probability of experiencing an adverse reaction to radiotherapy is due primarily to constitutive individual differences in normal tissue reactions and there is a suspicion that these have a strong genetic component. Several of the known breast cancer predisposition genes (e.g. BRCA1, BRCA2, ATM) affect pathways of DNA double strand break repair. DNA double strand breaks are the primary cytotoxic lesion induced by radiotherapy. This has led to concern that individuals who are genetically predisposed to breast cancer through carriage of variants in genes belonging to these pathways might also be at higher risk of suffering excessive normal tissue damage from radiotherapy.

The existence of constitutively radiosensitive individuals in the population means that radiotherapy dose rates for the majority of the patient population must be restricted, in order to keep the frequency of adverse reactions to an acceptable level. There is a clinical need, therefore, for reliable tests that can identify individuals who are at elevated risk for adverse reactions to radiotherapy. Such tests would indicate conservative or alternative treatments for individuals who are radiosensitive, while permitting escalation of radiotherapeutic doses for the majority of patients who are relatively radioresistant. It has been estimated that the dose escalations made possible by a test to triage breast cancer patients simply into radiosensitive, intermediate and radioresistant categories would result in an approximately 35% increase in local tumor control and consequent improvements in survival rates (Burnet et al., 1996).

Exposure to ionizing radiation is a proven factor contributing to oncogenesis in the breast (Dumitrescu and Cotarla 2005). Known breast cancer predisposition genes encode pathway components of the cellular response to radiation-induced DNA damage (Narod and Foulkes 2004). Accordingly, there is concern that the risk for second primary breast tumors may be increased by irradiation of normal tissues within the radiotherapy field. There does not appear to be any measurable increased risk for BRCA carriers from radiotherapy, however their risk for second primary tumors is already exceptionally high. There is evidence to suggest that risk for second primary tumors is increased in carriers in breast cancer predisposing alleles of the ATM and CHEK2 genes who are treated with radiotherapy (Bernstein et al., 2004; Broeks et al., 2004). It is expected that the risk of second primary tumors from radiotherapy (and, possibly, from intensive mammographic screening) will be better defined by obtaining accurate genetic risk profiles from patients during the treatment planning stage.

### Secondary Prevention

Approximately 30% of patients who are diagnosed with a stage 1 or 2 breast cancer will experience either a loco-regional or distant metastatic recurrence of their original tumor. Patients who have had a primary breast cancer are also at greatly increased risk for being diagnosed with a second primary tumor, either in the contralateral breast or in the ipsilateral breast when breast-conserving surgery has been carried out. Secondary prevention refers to methods used to prevent recurrences or second primary tumors from developing. Methods currently in use comprise; long-term treatment with Tamoxifen or another SERM either alone or alternated with an aromatase inhibitor, risk-reducing mastectomy of the contralateral breast, and risk-reducing oophorectomy (in patients who are at risk for familial breast-ovarian cancer). Considerations regarding the use of Tamoxifen have been discussed above. With risk-reducing surgical options, it is clear that the risk needs to be quantified as well as possible in order to make an informed cost:benefit analysis.

There are some indications that patients with known genetic predispositions to breast cancer fare worse than the majority of patients. Patients carrying the CHEK2 gene 1100delC variant have an estimated 2.8-fold increased risk of distant metastasis and a 3.9-fold increased risk of disease recurrence compared to non-carriers [de Bock, et al., (2004), J Med Genet, 41, 731-5]. Patients with BRCA1 node-negative tumors have a greater risk of metastasis than similar patients who do not carry a BRCA1 mutatation[Goffin, et al., (2003), Cancer, 97, 527-36; Moller, et al., (2002), Int J Cancer, 101, 555-9; Eerola, et al., (2001), Int J Cancer, 93, 368-72]. Genetic profiling can therefore be used to help assess the risk of local recurrence and metastasis, thereby guiding the choice of secondary preventative treatment.

In general, patients with a primary tumor diagnosis are at risk from second primary tumors at a constant annual incidence of 0.7% (Peto and Mack 2000). Patients with BRCA mutations are at significantly greater risks for second primary tumors than most breast cancer patients, with absolute risks in the range 40-60% (Easton 1999). Carriers of BRCA mutations have a greatly increased risk for second primary tumors [Stacey, et al., (2006), PLoS Med, 3, e217; Metcalfe, et al., (2004), J Clin Oncol, 22, 2328-35]. Patients with mutations in the CHEK2 gene have and estimated 5.7-fold increased risk of contralateral breast cancer [de Bock, et al., (2004), J Med Genet, 41, 731-5]. Carriers of the BARD1 Cys557Ser variant are 2.7 fold more likely to be diagnosed with a second primary tumor [Stacey, et al., (2006), PLoS Med, 3, e217]. Genetic risk profiling can be used to assess the risk of second primary tumors in patients and will inform decisions on how aggressive the preventative measures should be.

### Methods

Methods for risk assessment and diagnosis of breast cancer are described herein and are encompassed by the invention. The invention also encompasses methods of assessing an individual for probability of response to a therapeutic agent for breast cancer, as well as methods for predicting the effectiveness of a therapeutic agent for breast cancer. Kits for assaying a sample from a subject to detect susceptibility to breast cancer are also encompassed by the invention.

### Diagnostic and Screening Assays

In certain embodiments, the present invention pertains to methods of diagnosing, or aiding in the diagnosis of, breast cancer or a susceptibility to breast cancer, by detecting particular alleles at genetic markers that appear more frequently in breast cancer subjects or subjects who are susceptible to breast cancer. In a particular embodiment, the invention is a method of diagnosing a susceptibility to breast cancer by detecting at least one allele of at least one polymorphic marker (e.g., the markers described herein). The present invention describes methods whereby detection of particular alleles of particular markers or haplotypes is indicative of a susceptibility to breast cancer. Such prognostic or predictive assays can also be used to determine prophylactic treatment of a subject prior to the onset of symptoms of breast cancer. The present invention pertains in some embodiments to methods of clinical applications of diagnosis, e.g., diagnosis performed by a medical professional. In other embodiments, the invention pertains to methods of diagnosis or determination of a susceptibility performed by a layman. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively low cost. The resulting genotype information, made available to the individual, can be compared to information from the public literature about disease or trait risk associated with various SNPs. The diagnostic application of disease-associated alleles as described herein, can thus be performed either by the individual, through analysis of his/her genotype data, or by a health professional based on results of a clinical test. In other words, the diagnosis or assessment of a susceptibility based on genetic risk can be made by health professionals, genetic counselors or by the layman, based on information about his/her genotype and publications on various risk factors. In the present context, the term “diagnosing”, “diagnose a susceptibility” and “determine a susceptibility” is meant to refer to any available diagnostic method, including those mentioned above.

In addition, in certain other embodiments, the present invention pertains to methods of diagnosing, or aiding in the diagnosis of, a decreased susceptibility to breast cancer, by detecting particular genetic marker alleles or haplotypes that appear less frequently in breast cancer patients than in individual not diagnosed with breast cancer or in the general population.

As described and exemplified herein, particular marker alleles or haplotypes (e.g. markers located within the rs4848543 LD block, the rs13387042 LD block, the rs3803662 LD block, the STEAP3/TSAP6 gene, the markers and haplotypes as listed in Table 10, Table 15, Table 19, Table 20, Table 21 and Table 22, and markers in linkage disequilibrium therewith) are associated with breast cancer (e.g., All BC and/or MedPre breast cancer). In one embodiment, the marker allele or haplotype is one that confers a significant risk or susceptibility to breast cancer. In another embodiment, the invention relates to a method of diagnosing a susceptibility to breast cancer in a human individual, the method comprising determining the presence or absence of at least one allele of at least one polymorphic marker in a nucleic acid sample obtained from the individual, wherein the at least one polymorphic marker is selected from the group consisting of the polymorphic markers located within the rs4848543 LD block, the rs13387042 LD block, the rs3803662 LD block, the STEAP3/TSAP6 gene, the markers and haplotypes as listed in Table 10, Table 15, Table 19, Table 20, Table 21 and Table 22, and markers in linkage disequilibrium (e.g., defined as r²>0.2) therewith. In another embodiment, the invention pertains to methods of diagnosing a susceptibility to breast cancer in a human individual, by screening for at least one marker allele or haplotype, e.g. the markers located within the rs4848543 LD block, the rs13387042 LD block, the rs3803662 LD block, the STEAP3/TSAP6 gene, the markers and haplotypes as listed in Table 10, Table 15, Table 19, Table 20, Table 21 and Table 22, or markers in linkage disequilibrium therewith. In another embodiment, the marker allele or haplotype is more frequently present in a subject having, or who is susceptible to, breast cancer (affected, e.g. All BC or MedPre breast cancer), as compared to the frequency of its presence in a healthy subject (control, such as population controls). In another embodiment, the invention relates to a method of diagnosing a susceptibility to breast cancer in a human individual, the method comprising determining the presence or absence of at least one allele of at least one polymorphic marker in a nucleic acid sample obtained from the individual, wherein the at least one polymorphic marker is selected from marker rs4848543, marker rs13387042, marker rs3803662, and markers in linkage disequilibrium therewith. In certain embodiments, the significance of association of the at least one marker allele or haplotype is characterized by a p value<0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to breast cancer. These diagnostic methods involve detecting the presence or absence of at least one marker allele or haplotype that is associated with breast cancer. The haplotypes described herein include combinations of alleles at various genetic markers (e.g., SNPs, microsatellites). The detection of the particular genetic marker alleles that make up the particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by a breast cancer-associated nucleic acid (e.g., by protein sequencing or by immunoassays using antibodies that recognize such a protein). The marker alleles or haplotypes of the present invention correspond to fragments of a genomic DNA sequence associated with breast cancer. Such fragments encompass the DNA sequence of the polymorphic marker or haplotype in question, but may also include DNA segments in strong LD (linkage disequilibrium) with the marker or haplotype (e.g., as determined by a value of r² greater than 0.2 and/or |D′|>0.8).

In one embodiment, diagnosis of a susceptibility to breast cancer can be accomplished using hybridization methods, such as Southern analysis, Northern analysis, and/or in situ hybridizations (see Current Protocols in Molecular Biology, Ausubel, F. et al., eds., John Wiley & Sons, including all supplements). A biological sample from a test subject or individual (a “test sample”) of genomic DNA, RNA, or cDNA is obtained from a subject suspected of having, being susceptible to, or predisposed for breast cancer (the “test subject”). The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined. The presence of a specific marker allele can be indicated by sequence-specific hybridization of a nucleic acid probe specific for the particular allele. The presence of more than specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to breast cancer, a hybridization sample is formed by contacting the test sample containing an breast cancer-associated nucleic acid, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence as set forth in SEQ ID NO:4, SEQ IN NO:5 or SEQ ID NO:6, the nucleotide sequence of the rs4848543 LD block, the rs13387042 LD block, the rs3803662 LD block, and/or the STEAP3/TSAP gene, as described herein, optionally comprising at least one allele of a marker described herein, or at least one marker or haplotype described herein (e.g., the markers and haplotypes as listed in Table 10, Table 15, Table 19, Table 20, Table 21 and Table 22, and markers in linkage disequilibrium therewith), or the probe can be the complementary sequence of such a sequence. In a particular embodiment, the nucleic acid probe is a portion of the nucleotide sequence as set forth in SEQ ID NO:4, SEQ ID NO:5, or SEQ ID NO:6; or the nucleotide sequence of the rs4848543 LD block, the rs13387042 LD block, the rs3803662 LD block and/or the STEAP3/TSAP6 gene, as described herein, optionally comprising at least one allele of a marker described herein (e.g., the markers and haplotypes as listed in Table 10, Table 15, Table 19, Table 20, Table 21 and Table 22, and markers in linkage disequilibrium therewith), or at least one allele contained in the haplotypes described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is then detected using standard methods. If specific hybridization occurs between the nucleic acid probe and the breast cancer-associated nucleic acid in the test sample, then the sample contains the allele that is complementary to the nucleotide that is present in the nucleic acid probe. The process can be repeated for other markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to breast cancer.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another hybridization method, Northern analysis (see Current Protocols in Molecular Biology, Ausubel, F. et al., eds., John Wiley & Sons, supra) is used to identify the presence of a polymorphism associated with breast cancer. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with breast cancer. Hybridization of the PNA probe is thus diagnostic for breast cancer or a susceptibility to breast cancer.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype associated with breast cancer can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.), The technique can assess the presence of an alteration in the expression or composition of a polypeptide or splicing variant(s) that is encoded by a nucleic acid associated with breast cancer. Further, the expression of the variant(s) can be quantified as physically or functionally different.

In another method of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles at polymorphic sites associated with breast cancer (e.g. the polymorphic markers and haplotypes of Table 10, Table 15, Table 19, Table 20, Table 21 and/or Table 22, and markers in linkage disequilibrium therewith). Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid associated with breast cancer, and the presence of a specific allele can then be detected directly by sequencing the polymorphic site (or multiple polymorphic sites in a haplotype) of the genomic DNA in the sample.

Allele-specific oligonucleotides can also be used to detect the presence of a particular allele at a breast cancer-associated nucleic acid (e.g. the polymorphic markers and haplotypes of Table 10, Table 15, Table 19, Table 20, Table 21 and Table 22 and markers in linkage disequilibrium therewith), through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a breast cancer-associated nucleic acid, and which contains a specific allele at a polymorphic site (e.g., a marker or haplotype as described herein). An allele-specific oligonucleotide probe that is specific for one or more particular a breast cancer-associated nucleic acid can be prepared using standard methods (see, e.g., Current Protocols in Molecular Biology, supra). PCR can be used to amplify the desired region. The DNA containing the amplified region can be dot-blotted using standard methods (see, e.g., Current Protocols in Molecular Biology, supra), and the blot can be contacted with the oligonucleotide probe. The presence of specific hybridization of the probe to the amplified region can then be detected. Specific hybridization of an allele-specific oligonucleotide probe to DNA from the subject is indicative of a specific allele at a polymorphic site associated with breast cancer (see, e.g., Gibbs, R. et al., Nucleic Acids Res., 17:2437-2448 (1989) and WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tₘ) of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from a subject, can be used to identify polymorphisms in a nucleic acid associated with breast cancer (e.g. the polymorphic markers and haplotypes of Table 10, Table 15, Table 19, Table 20, Table 21 and Table 22 and markers in linkage disequilibrium therewith). For example, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art (see, e.g., U.S. Pat. No. 5,143,854, PCT Patent Publication Nos. WO 90/15070 and 92/10092). These arrays can generally be produced using mechanical synthesis methods that incorporate a combination of photolithographic methods and solid phase oligonucleotide synthesis methods, or by other methods known to the person skilled in the art (see, e.g., Fodor, S. et al., Science, 251: 767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is allowed to hybridize with the array. Detection of hybridization is a detection of a particular allele in the nucleic acid of interest. Hybridization and scanning are generally carried out by methods described herein or by other methods known to those skilled in the art, for example as described in published PCT Application Nos. WO 92/10092 and WO 95/11995, and U.S. Pat. No. 5,424,186, the entire teachings of each of which are incorporated by reference herein. In brief, a target nucleic acid sequence, which includes one or more previously identified polymorphic markers, is amplified by well-known amplification techniques (e.g., PCR). Typically this involves the use of primer sequences that are complementary to the two strands of the target sequence, both upstream and downstream, from the polymorphic site. Asymmetric PCR techniques can also be used. Amplified target, generally incorporating a label, is then allowed to hybridize with the array under appropriate conditions that allow for sequence-specific hybridization. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphic site, arrays can include multiple detection blocks, and thus be capable of analyzing multiple, specific polymorphisms (e.g., multiple polymorphisms of a particular haplotype). In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of both of which are incorporated by reference herein.

Other methods of nucleic acid analysis that are available to those skilled in the art can be used to detect a particular allele at a polymorphic site associated with breast cancer (e.g. the polymorphic markers and haplotypes of Table 10, Table 15, Table 19, Table 20, Table 21 and Table 22 and markers in linkage disequilibrium therewith). Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of breast cancer or a susceptibility to breast cancer can be made by examining expression and/or composition of a polypeptide encoded by breast cancer-associated nucleic acid in those instances where the genetic marker(s) or haplotype(s) of the present invention result in a change in the composition or expression of the polypeptide. Thus, diagnosis of a susceptibility to breast cancer can be made by examining expression and/or composition of one of these polypeptides, or another polypeptide encoded by a breast cancer-associated nucleic acid, in those instances where the genetic marker or haplotype of the present invention results in a change in the composition or expression of the polypeptide. The haplotypes and markers of the present invention that show association to breast cancer may play a role through their effect on one or more of these nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

Thus, in another embodiment, the variants (markers or haplotypes) of the invention showing association to breast cancer affect the expression of a nearby gene. It is well known that regulatory element affecting gene expression may be located tenths or even hundreds of kilobases away from the promoter region of a gene. By assaying for the presence or absence of at least one allele of at least one polymorphic marker of the present invention, it is thus possible to assess the expression level of such nearby genes. It is thus contemplated that the detection of the markers or haplotypes of the present invention can be used for assessing expression for one or more of the STEAP3/TSAP6, LOC643714, and TNRC9 genes.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a breast cancer-associated nucleic acid. An alteration in expression of a polypeptide encoded by a breast cancer-associated nucleic acid can be, for example, an alteration in the quantitative polypeptide expression (i.e., the amount of polypeptide produced). An alteration in the composition of a polypeptide encoded by a breast cancer-associated nucleic acid is an alteration in the qualitative polypeptide expression (e.g., expression of a mutant polypeptide or of a different splicing variant). In one embodiment, diagnosis of a susceptibility to breast cancer is made by detecting a particular splicing variant encoded by a nucleic acid associated with breast cancer, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of polypeptide encoded by a breast cancer-associated nucleic acid in a control sample. A control sample is a sample that corresponds to the test sample (e.g., is from the same type of cells), and is from a subject who is not affected by, and/or who does not have a susceptibility to, breast cancer. In one embodiment, the control sample is from a subject that does not possess a marker allele or haplotype as described herein. Similarly, the presence of one or more different splicing variants in the test sample, or the presence of significantly different amounts of different splicing variants in the test sample, as compared with the control sample, can be indicative of a susceptibility to breast cancer. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a breast cancer-associated nucleic acid can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a breast cancer-associated nucleic acid can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a nucleic acid associated with breast cancer (e.g., a nucleic acid encoding the STEAP3/TSAP6, LOC643714 or TNRC9 genes) in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, the diagnosis of a susceptibility to breast cancer is made by detecting at least one marker or haplotype of the present invention (e.g., associated alleles of the markers and haplotypes of Table 10, Table 15, Table 19, Table 20, Table 21 and Table 22 and markers in linkage disequilibrium therewith) in combination with an additional protein-based, RNA-based or DNA-based assay. The methods of the invention can also be used in combination with an analysis of a subject's family history and risk factors (e.g., environmental risk factors, lifestyle risk factors).

### Kits

Kits useful in the methods of the invention comprise components useful in any of the methods described herein, including for example, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with breast cancer, means for analyzing the nucleic acid sequence of a nucleic acid associated with breast cancer, means for analyzing the amino acid sequence of a polypeptide encoded by a nucleic acid associated with breast cancer, etc. The kits can for example include necessary buffers, nucleic acid primers for amplifying nucleic acids of the invention (e.g., one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with breast cancer diagnostic assays.

In one embodiment, the invention is a kit for assaying a sample from a subject to detect the presence of a breast cancer or a susceptibility breast cancer in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism, wherein the polymorphism is selected from the group consisting of the polymorphisms as listed in Table 10, Table 15, Table 19, Table 20, Table 21 and Table 22, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are indicative of breast cancer. In another embodiment, the kit comprises one or more labeled nucleic acids capable of allele-specific detection of one or more specific polymorphic markers or haplotypes associated with breast cancer, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers in Table 10, Table 15, Table 19, Table 20, Table 21 and Table 22. In one embodiment, the marker to be detected is selected from the markers in Table 10, Table 15 or Table 19. In one embodiment, the marker to be detected is selected from the markers in Table 10. In another embodiment, the marker to be detected is selected from the markers in Table 15 In another embodiment, the marker to be detected is selected from the markers in Table 19. In a preferred embodiment, the marker to be detected is rs4848543, rs13387042 and/or rs3803662. Other preferred embodiments include those that include reagents for detecting marker rs4848543, those that include reagents for detecting marker rs13387042 and those that include reagents for detecting marker rs3803662. In another embodiment, the marker or haplotype to be detected comprises at least one marker from the group of markers in strong linkage disequilibrium, as defined by values of r² greater than 0.2, to at least one of the group of markers consisting of the markers listed in Table 10, Table 15, Table 19, Table 20, Table 21 and Table 22. In yet another embodiment, the marker or haplotype to be detected comprises at least one marker selected from the group of markers consisting of markers rs4848543, rs13387042 and rs3803662, and markers in linkage disequilibrium therewith.

In one of such embodiments, the presence of the marker or haplotype is indicative of a susceptibility (increased susceptibility or decreased susceptibility) to breast cancer. In another embodiment, the marker is rs4848543 allele A, rs13387042 allele A, and/or rs3803662 allele T, the presence of which is indicative of increased risk of breast cancer (e.g., All BC and/or MedPre breast cancer). In still another embodiment, the presence of the marker or haplotype is indicative of response to a breast cancer therapeutic agent. In another embodiment, the presence of the marker or haplotype is indicative of breast cancer prognosis. In yet another embodiment, the presence of the marker or haplotype is indicative of progress of breast cancer treatment. Such treatment may include intervention by surgery, medication, radiation therapy or by other means (e.g., lifestyle changes).

Diagnosis of Breast Cancer Associated with Polymorphisms of the Invention

Although the methods of diagnosis have been generally described in the context of diagnosing susceptibility to breast cancer, the methods can also be used to diagnose breast cancer associated with the polymorphic markers of the present invention. For example, an individual having breast cancer or risk factors associated with breast cancer can be assessed to determine whether the presence in the individual of a polymorphism or haplotype of the present invention could have been a contributing factor to breast cancer in the individual. In one embodiment, identification of breast cancer associated with the markers and/or haplotypes of the present invention facilitates treatment planning. For example, preventive treatment to minimize the occurrence of the individual developing breast cancer can be administered. Such preventive treatment can also include assessment of whether the individual is heterozygous or homozygous for the at-risk variants of the invention. In other embodiments of the invention, therapies can be designed and therapeutics selected to target the appropriate gene or protein associated with the polymorphism and/or haplotype of the present invention

In other embodiments, the invention pertains to a method for the diagnosis and identification of breast cancer associated with the polymorphisms of the invention in a subject, by identifying the presence of a polymorphic marker or haplotype of the invention, as described in detail herein. For example, the polymorphic markers and/or haplotypes described herein are found more frequently in subjects with breast cancer than in subjects not affected by breast cancer. Therefore, these markers and/or haplotypes have predictive value for diagnosing breast cancer. In one embodiment, the marker or haplotype having predictive value for detecting breast cancer comprises one or more markers selected from the group consisting of the markers in Table 10, Table 15, Table 19, Table 20, Table 21 and Table 22. In another embodiment, the marker having predictive value for diagnosing breast cancer is selected from markers rs4848543, rs13387042 and rs3803662, and markers in linkage disequilibrium therewith.

In another embodiment, the marker allele having predictive value for diagnosing breast cancer comprises rs4848543 allele A, rs13387042 allele A, and rs3803662 allele T, and markers in linkage disequilibrium therewith.

The methods described herein can thus be used to assess a sample from a subject for the presence or absence of a specific allele of a marker, or a haplotype; the presence or absence of a specific allele of a marker, or a haplotype, is indicative of a susceptibility to breast cancer.

In one embodiment of the invention, diagnosis of breast cancer associated with the polymorphisms and/or haplotypes of the present invention is made by detecting a polymorphism of the present invention. Particular polymorphisms are described herein (see, e.g., Table 10, Table 15, Table 19, Table 20, Table 21 and Table 22, and markers in linkage disequilibrium therewith). A test sample of genomic DNA, RNA, or cDNA, is obtained from a subject having breast cancer to determine whether the disease is associated with one or more polymorphisms of the invention. The DNA, RNA or cDNA sample is then examined to determine whether a specific allele of a polymorphism or a specific haplotype of the invention is found to be present in the sample. If the nucleic acid sample is found to contain the specific allele of the polymorphism or the haplotype, then the presence of the allele or haplotype is indicative of breast cancer associated with the polymorphism and/or haplotype.

Methods known to the person skilled in the art, including but not limited to hybridization methods, such as Southern analysis, Northern analysis, quantitative PCR, in situ hybridization, restriction digestion or sequence analysis, can be used to detect the polymorphism. Diagnosis of breast cancer associated with the polymorphisms of the invention can also be made by examining expression and/or composition of a polypeptide encoded by a nucleic acid associated with the polymorphisms of the invention, using a variety of methods, including enzyme linked immunosorbent assays (ELISAs), Western blots, immunoprecipitations and immunofluorescence, or other methods well known to the skilled person.

### Therapeutic Agents

Variants of the present invention (e.g., the markers and/or haplotypes of the invention, e.g., the markers listed in any one of Table 10, Table 15 and Table 19) can be used to identify novel therapeutic targets for breast cancer. For example, genes containing, or in linkage disequilibrium with, variants (markers and/or haplotypes) associated with breast cancer, or their products (e.g., one or more of the STEAP3/TSAP6 gene, the LOC643714 gene, and/or the TNRC9 gene), as well as genes or their products that are directly or indirectly regulated by or interact with these variant genes or their products, can be targeted for the development of therapeutic agents to treat breast cancer. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol, Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used to treat breast cancer. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEES Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants of the present invention (e.g., the markers and haplotypes set forth in Table 10, Table 15 and Table 19) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev, Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of breast cancer, or a genetic defect causing breast cancer, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat and/or prevent breast cancer. Thus, the variants of the invention are useful as targets for the identification and/or development of therapeutic agents. In certain embodiments, such methods include assaying the ability of an agent or compound to modulate the activity and/or expression of a nucleic acid that includes at least one of the variants (markers and/or haplotypes) of the present invention, or the encoded product of the nucleic acid, for example one or more of the STEAP3/TSAP6 gene, the LOC643714 gene, and/or the TNRC9 gene. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level, or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating and/or preventing breast cancer can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents, Methods of Monitoring Progress of Treatment and Methods for Treating Breast Cancer

As is known in the art, individuals can have differential responses to a particular therapy (e.g., a therapeutic agent or therapeutic method, as described further herein). The basis of the differential response may be genetically determined in part. Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype is indicative of a different response rate to a particular treatment modality. This means that a patient diagnosed with breast cancer, and carrying a certain allele at a polymorphic or haplotype of the present invention (e.g., the at-risk and protective alleles and/or haplotypes of the invention) would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype at (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed).

Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

As described further herein, current clinical preventive options for breast cancer are mainly chemopreventive (chemotherapy, or hormonal therapy) and prophylactic surgery. The most common chemopreventive is Tamoxifen and Raloxifene; other options include the aromatase inhibitors. Treatment options also include radiation therapy, for which a proportion of patients experience adverse symptoms. The markers of the invention, as described herein, may be used to assess response to these therapeutic options, or to predict the progress of therapy using any one of these treatment options. Thus, genetic profiling can be used to select the appropriate treatment strategy based on the genetic status of the individual, or it may be used to predict the outcome of the particular treatment option, and thus be useful in the strategic selection of treatment options or a combination of available treatment options.

The present invention also relates to methods of monitoring progress or effectiveness of a treatment for a breast cancer. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for breast cancer as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of the at-risk variants of the present invention, i.e. individuals who are carriers of at least one allele of at least one polymorphic marker conferring increased risk of developing breast cancer may be more likely to respond to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Computer-Implemented Aspects

The present invention also relates to computer-implemented applications of the polymorphic markers and haplotypes described herein to be associated with breast cancer. Such applications can be useful for storing, manipulating or otherwise analyzing genotype data that is useful in the methods of the invention, as described herein. One example pertains to storing genotype information derived from an individual on readable media, so as to be able to provide the genotype information to a third party (e.g., the individual), or for deriving information from the genotype data, e.g., by comparing the genotype data to information about genetic risk factors contributing to increased susceptibility to breast cancer, and reporting results based on such comparison.

One such aspect relates to computer-readable media. In general terms, such medium has capabilities of storing (i) identifier information for at least one polymorphic marker or a haplotye; (ii) an indicator of the frequency of at least one allele of said at least one marker, or the frequency of a haplotype, in individuals with breast cancer; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

Additional information about the individual can be stored on the medium, such as ancestry information, information about sex, physical attributes or characteristics (including height and weight), biochemical measurements, treatment administration, treatment outcome, medication, or other useful information that is desirable to store or manipulate in the context of the genotype status of a particular individual.

The invention furthermore relates to an apparatus that is suitable for determination or manipulation of genetic data useful for determining a susceptibility to breast cancer in a human individual. Such an apparatus can include a computer-readable memory, a routine for manipulating data stored on the computer-readable memory, and a routine for generating an output that includes a measure of the genetic data. Such measure can include values such as allelic or haplotype frequencies, genotype counts, sex, age, phenotype information, values for odds ratio (OR) or relative risk (RR), population attributable risk (PAR), or other useful information that is either a direct statistic of the original genotype data or based on calculations based on the genetic data.

The markers and haplotypes shown herein to be associated with increased susceptibility (e.g., increased risk) of breast cancer, are in certain embodiments useful for interpretation and/or analysis of genotype data. Thus in certain embodiments, an identification of an at-risk allele or an at-risk haplotype for breast cancer, as shown herein, or an allele at a polymorphic marker or a haplotype in LD with any one of the markers and/or haplotypes shown herein to be associated with breast cancer, is indicative of the individual from whom the genotype data originates is at increased risk of breast cancer. In one such embodiment, genotype data is generated for at least one polymorphic marker shown herein to be associated with breast cancer, or a marker in linkage disequilibrium therewith. The genotype data is subsequently made available to a third party, such as the individual from whom the data originates, for example via a user interface accessible over the internet, together with an interpretation of the genotype data, e.g., in the form of a risk measure (such as an absolute risk (AR), risk ratio (RR) or odds ration (OR)) for the disease (e.g., breast cancer. In another embodiment, at-risk markers identified in a genotype dataset derived from an individual are assessed and results from the assessment of the risk conferred by the presence of such at-risk variants in the dataset are made available to the individual, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived. In particular embodiments, the results of risk assessment is made available to a third party, e.g., a physician, other healthcare worker or genetic counselor.

### Markers Useful in Various Aspects of the Invention

The above-described methods and applications can all be practiced with the markers and haplotypes of the invention that have in more detail been described herein in general terms as being useful for assessing susceptibility to breast cancer. Thus, these applications can in general be reduced to practice using markers as set forth in any of the Tables 10, 15, 19, 20, 21 and 22, and markers in linkage disequilibrium therewith. In certain embodiments, the markers or haplotypes are present within the genomic segments whose sequences are set forth in SEQ ID NO:4, SEQ ID NO:5 or SEQ ID NO:6. In certain embodiments, the markers are present within the rs4848543 LD block, the rs13387042 LD block or the rs3803662 LD block. In particular embodiments, the markers are selected from the markers set forth in Table 10. In other embodiments, the markers are selected from the markers set forth in Table 15. In other embodiments, the markers are selected from the markers set forth in Table 19. In some embodiments, the markers are selected from the markers set forth in Tables 7 and 8. In some other embodiments, the markers are selected from the markers set forth in Table 14. In some other embodiments, the markers are selected from the markers set forth in Table 18. In other embodiments, the markers are one of rs4848543 (SEQ ID NO:1), rs13387042 (SEQ ID NO:2), or rs3803662 (SEQ ID NO:3), optionally including markers in linkage disequilibrium therewith. In one embodiment, the markers are one of rs4848543 (SEQ ID NO:1), rs13387042 (SEQ ID NO:2), and rs3803662 (SEQ ID NO:3). In certain preferred embodiments, the marker is rs4848543 (SEQ ID NO:1). In other preferred embodiments, the marker is rs13387042 (SEQ ID NO:2). In other preferred embodiments, the marker is rs3803662 (SEQ ID NO:3). In particular embodiments, linkage disequilibrium is defined by numerical values for r² of greater than 0.2. In another embodiment, the marker or haplotype comprises at least one marker selected from rs4848543 allele A, rs13387042 allele A, and rs3803662 allele T.

### Nucleic Acids and Polypeptides

The nucleic acids and polypeptides described herein can be used in methods and kits of the present invention, as described in the above. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions 100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, a nucleotide sequence comprising the polymorphic markers listed Table 10, Table 15, Table 19, Table 20, Table 21 and Table 22, and markers in linkage disequilibrium therewith, and the nucleotide sequence of the STEAP3/TSAP6, LOC643714 and TNRC9 genes, or a nucleotide sequence comprising, or consisting of, the complement of the nucleotide sequence of a nucleotide sequence comprising the polymorphic markers listed in Table 10, Table 15, Table 19, Table 20, Table 21 and Table 22, and markers in linkage disequilibrium therewith, and the nucleotide sequence of the STEAP3/TSAP6, LOC643714 and TNRC9 genes, or fragments thereof, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify breast cancer or a susceptibility to breast cancer, and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunization techniques, and/or as an antigen to raise anti-DNA antibodies or elicit immune responses.

### Antibodies

Polyclonal antibodies and/or monoclonal antibodies that specifically bind one form of the gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an, immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular breast cancer. Antibodies specific for a variant protein of the present invention that is encoded by a nucleic acid that comprises at least one polymorphic marker or haplotype as described herein can be used to screen for the presence of the variant protein, for example to screen for a predisposition to breast cancer as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting example.

### Exemplification

Patient and Control Selection: Approval for this study was granted by the National Bioethics Committee of Iceland and the Icelandic Data Protection Authority. Records of breast cancer diagnoses were obtained from the Icelandic Cancer Registry (ICR). Records included all cases of invasive breast tumors and ductal or lobular carcinoma in-situ diagnosed in Iceland from 1 Jan. 1955 to 31 Dec. 2005. The ICR contained records of 4603 individuals diagnosed during this period. A prevalence cohort comprising all living patients (approximately 2840) were eligible for recruitment into the study. We obtained informed consent, a blood sample, and clinical information from 2210 patients, a yield of approximately 78%. The initial group of controls was comprised of all individuals from other ongoing deCODE Genetics genome-wide association studies and who had been genotyped on Illumina SNP chips by the time the breast cancer patient group had been genotyped. Individuals with entries in the Icelandic Cancer Registry indicating a diagnosis of breast, prostate or colorectal cancer up to the end of 2005 were excluded from the control group, resulting in a set of 4477 controls. An independent replication set of controls was comprised of a further 7406 individuals from non-breast cancer projects who were typed after the 4477 set. In this 7406 set, prostate and colorectal cancer patients were not excluded because we had obtained interim data indicating that the frequencies of the SNPs in question did not differ from controls in these patient samples. In each control sets there was no difference between genders in the frequencies of the SNPs listed in Tables 8,9, & 14 below. Therefore these control groups provided a reasonable representation of the population frequencies of the SNPs under investigation.

Genealogical Database: deCODE Genetics maintains a computerized database of the genealogy of Icelanders. The records include almost all individuals born in Iceland in the last two centuries and for that period around 95% of the parental connections are known [Sigurdardottir, et al., (2000), Am J Hum Genet, 66, 1599-609]. In addition, a county of residence identifier is recorded for most individuals, based on census and parish records. The information is stored in a relational database with encrypted personal identifiers that match those used on the biological samples and ICR records, allowing cross-referencing of the genotypes and phenotypes of the study participants with their genealogies.

Sample Handling: Blood samples were preserved in EDTA at −20° C. DNA was isolated from whole blood using a Qiagen (http://www.quiagen.com) extraction column method. DNA was stored at 4° C.

Genotyping: A set of 1600 patient samples was then randomly selected and genotyped on Illumina Infinium Hap300 SNP bead microarrays (Illumina, San Diego, Calif., USA), containing 317,511 SNPs derived from Phase I of the International HapMap project. 4480 cancer-free control samples were genotyped on the same platform. Of the total number of SNPs on the chip, 170 generated no genotypes and an additional 24 SNPs had yields lower than 80%. An additional 61 SNPs showed very significant distortion from Hardy-Weinberg equilibrium in the controls (p<1×10⁻¹⁰). 104 SNPs were monomorphic and 58 SNP's were nearly monomorphic (i.e. the minor allele frequency in the combined cohort of patients and controls was less than 0.001). Lastly, 5 markers were determined to have genotyping problems after investigation of particular regions and possible signals in several different on-going genomewide association studies inhouse. All of these problematic SNPs were removed from the analysis. Thus, the final analyses presented in the text utilizes 317,089 SNPs. Any chips with a call rate below 98% were also excluded from the analysis.

The sequence context of the three key SNPs examined in this study are given below:

rs4848543 (SEQ ID NO: 1):
caggataagattatttctgaggctgcttccatcagtggttctgtgagctggcccatgtattcagccagtcagtaggccatcaagcatgcatct
acgcaaggcactagaagggttacatacaatgggaaactgaggcccagaaggaggaaagatgaagtgcccaaagttaaggagataaa
agcagaagtgcaggtaagagaaggagtattcatattactgcaggaatccaaattctacaagaacatacgcttgttctttaaagggagccc
tcgctttaagcattatttgccgatcaatacc[A/C]attaataattacaggacatgggcaaatattctagcaaaggtcctgaggtcttcctag
atgtgcccaaaataacaaggactccccccgcaggtggcaccaaaagtcggaggatgtgttggaagctgaccttccccacctgtccctgcc
tcacccacagtcacccacccctctaggatgtaatggtgaatcattcaacacatgggccaccttgggaagtcgcttaacctctctcccttttcac
gttataaaatgggcatagtcattacacaaatgcataaatcaacctcaattttctcatct
rs13387042 (SEQ ID NO: 2):
ctgacatagccgcctgtgcaggtttttgggaccaggtctgccatctgtattcccaggctgggctggagcacctgaggctactatggttacag
aaaggaacctggcatccgaaggtataggctcaaatcccagctctcatgattgctagctttgaggtttcaggttcatttcttaagtgctctgag
ctttggtgccctcctcttcaaaatgggaatgaggagcatgtctagctccctgttttgttgcagtgaatagctacatgaagaacagctaaacc
agaacagaaagaaggcaaatgga[A/G]gctacagaaaccaaggatttccttgttgaatcgaatcttccttcaatcttccttcaccacact
agtggatctccctgtgggagggatgttgagagtgctccgtgttttttttgttttttttttttttttttttgtgatggagtctcactctgtcaccc
agactggaatgcagtggtgcgatctcggctcactgcaacctctgcctcccaggttcaagtgattctcctgcctcagcttcccaagtagccagga
ctacaggcatatgccatcacacccagctaatttttgtatttttagtag
rs3803662 (SEQ ID NO: 3):
gctagtccttggctgttctgtgatcaaatgcaattgtttcccatttcttaagaaacagaatctttattagactcagttcttttcttggttatttt
tacaattttattgttctatggttattaaaaaataacatgtcatataaattaagcctagaaacatggatgtttatcaaagaaaggattgtcatcca
aagcaccaactatgagagatatctatgtgcaatggtatatagatctgtcatagaagggtttaattatatctgcctaatgattttctctccttaat
gcctctatagctgtc[C/T]cttagcgaagaataaaactgtggactgacccccacccatttgcgaagaaagtactgggtcttcagctttcattg
ttcagccggtggtctttgtggacaacaccaggggcccctcctgctgaagactgtctggacttcctgattttcactcctgtataaaccccctccc
ccattactacctttgtctccttggggaaaaaaaatgtttttcacttaagcaaaataataactacccagtatttgtttctggagtgaaattgtaa
acttcattgtacattcttcctggctttgatgcttatcg

We designed and tested assays to detect a number of SNPs in the rs4848543 LD block. These assays were based on the Centaurus SNP assay technology described in [Kutyavin, et al., (2006), Nucleic Acids Res, 34, e128]. The assays, and their primers were:

SG02S733
rs895398
(Build 34) chr2: 120,058,180
Forward Primer: TTGGAACCTCCCACTGCCACA
Reverse Primer: ACAGCCCAGGTTTCAGGTTGGCAT
VIC-Probe: AGCGACACTG
FAM-Probe: GCGACACCG
Enhancer: CCTG*AGATCCAGGCA
SG02S738
rs4848543
(Build 34) chr2: 120,061,096
Forward Primer: AGGGAGCCCTCGCTTTAAGCATTA
Reverse Primer: TTTGGGCACATCTAGGAAGACCTC
VIC-Probe: GAT*CAATACCC
FAM-Probe: CGAT*CAATACCA
Enhancer: TTAATAATTACAGGACATG
SG02S739
rs6759589
(Build 34) chr2: 120,064,976
Forward Primer: TCAGCAGCCTCTTGTCTCACTAAT
Reverse Primer: CTTCTTGCCCAGGCAGGACAGA
VIC-Probe: TAGACTGAGCC
FAM-Probe: AAT*AGACTAAGCC
Enhancer: CCATCCCCAACTAA
SG02S753
rs895397
(Build 34) chr2: 120,072,400
Forward Primer: CACTACTAGATCATGGGAGATGCCT
Reverse Primer: GTTTCCTCAGTTGTAATGTAGACC
VIC-Probe: TT*T*A*ACCCTT
FAM-Probe: GTTTTAACCCCT
Enhancer: GCTGCTCGGAAG
SG02S740
rs838102
(Build 34) chr2: 120,079,878
Forward Primer: GGTTTTGCATCCTTGCTCACTCACAT
Reverse Primer: GTGCCCTCTGCGTACTTGCGAAT
VIC-Probe: TACTGCCACC
FAM-Probe: CTTACTGCCACT
Enhancer: TGTCTCCTCACTC
SG02S741
rs838100
(Build 34) chr2: 120,083,710
Forward Primer: ACTTACCAACAGCAGGCTGGTG
Reverse Primer: CCAGAAAGGGTGGCTCACCTCA
VIC-Probe: GTCTGAGGCT
FAM-Probe: GGTCTGAGGTT
Enhancer: TCAGGTCACTCG
SG02S734
rs12711924
(Build 34) chr2: 120,107,316
Forward Primer: AACGCTTATCACAGAGCCAGGTAG
Reverse Primer: GGAAAACCATGAACACTGTGGCAA
VIC-Probe: CCCGCCGT
FAM-Probe: GCCCGCCAT
Enhancer: TTGTCTTTCTTGTGTC
SG02S742
rs3731603
(Build 34) chr2: 120,117,062
Forward Primer: AACA*CTCTAGGGAGTAAAGCTC
Reverse Primer: GAGGA*CATTTGGAGTCCCCAAT
VIC-Probe: GACTGTCTTCAA
FAM-Probe: ACTGT*CT*TCAC
Enhancer: AAGTCAGTCCTGAG
SG02S728
rs13387042
(Build 34) chr2: 218,108,374
Forward Primer: CCACTAGTGTGGTGA*AGGAAGATT
Reverse Primer: GCTACATGA*AGAA*CAGCTAAACC
VIC-Probe: TTTCTGTAGCCT
FAM-Probe: TT*T*CTGTAGCT*T
Enhancer: CATTTGCCTTCTTTC

*indicates a modified base, as described
[Kutyavin, et al., (2006), Nucleic Acids Res,
34, e128].

A single founder mutation of the BRCA2 gene (999del5) is present at a carrier frequency of 0.6%-0.8% in the general Icelandic population and 7.7%-8.6% in female breast cancer patients [Thorlacius, et al., (1997), Am J Hum Genet, 60, 1079-84; Gudmundsson, et al., (1996), Am J Hum Genet, 58, 749-56]. The possible presence of BRCA2 999del5 mutations was determined in 1499 of the cases using a microsatellite-type PCR assay, designated DG13S3727. Primers used were

Forward TGTGAAAAGCTATTTTTCCAATC
Reverse: ATCACGGGTGACAGAGCAA

The sequence amplified by DG13S3727 is:

DG13S3727 location = chr13: 30703058-30703261
tgtgaaaagctatttttccaatcatgatgaaagtctgaagaaaaatgata
gatttatcgcttctgtgacagacagtgaaaacacaaatcaaagagaagct
gcaagtcatggtaagtcctctgtttagttgaactacaggtttttttgttg
ttgttgttttgattttttttttttgaggtggagtcttgctctgtcacccg
tgat

Statistical Methods: Assuming the multiplicative model, we calculate the relative risk (RR) of a SNP allele as RR=[p/(1−p)]/[s/(1−s)] where p and s are the frequencies of the variant in the patients and in the controls respectively. P-values associated with RR's were calculated based on a standard likelihood ratio Chi-squared statistic. Confidence intervals were calculated assuming that the estimate of RR has a log-normal distribution.

Haplotype frequencies were estimated by maximum likelihood and tests of differences between cases and controls, performed using a generalized likelihood ratio test. The haplotype analysis program called NEMO, which stands for NEsted Models, was used to calculate all the haplotype results. To handle uncertainties with phase and missing genotypes, it is emphasized that NEMO does not use a common two-step approach to association tests, where haplotype counts are first estimated, possibly with the use of the EM algorithm, and then tests are performed treating the estimated counts as though they are true counts, a method that can sometimes be problematic and may require randomization to properly evaluate statistical significance. Instead, with NEMO, maximum likelihood estimates, likelihood ratios and p-values are computed directly for the observed data, and hence the loss of information due to uncertainty in phase and missing genotypes is automatically captured by the likelihood ratios.

Some Icelandic patients and controls are related, both within and between groups. To correct the association results for relatedness, we simulated genotypes by propagating alleles through the Icelandic genealogy. For each simulation we performed the case-control association test in the same way as for the real genotypes, i.e. the Chi-squared likelihood ratio test. We calculated the average of the Chi-squared statistics over N simulations (which is identical to the variance of the corresponding Z-score), which for independent individuals under the null hypothesis has to be 1, whereas for related individuals it is expected to be slightly larger. The correction for relatedness was then applied by dividing the Chi-square statistics in the real association by the average obtained from the simulations.

Joint analyses of multiple case-control replication groups were carried out using a Mantel-Haenszel model in which the groups were allowed to have different population frequencies for alleles, haplotypes and genotypes but were assumed to have common relative risks.

All P-values are reported as two-sided.

Analysis of Multiple Primary Breast Tumors: Diagnostic records of multiple primary breast cancer (MPBC) were obtained from the ICR. Primary tumors diagnosed in addition to the first breast cancer diagnosis were confirmed both clinically and by histology to be independent primary tumors, arising simultaneously or subsequently to the first breast cancer and occurring in the contra lateral or ipsilateral breast. Tumors that were classified clinically or by histology as recurrences of the original tumor or multifocal single primary tumors were excluded. Patients with diagnoses of 2 or more independent primary tumors appearing in the registry data up until the end of December 2005 were considered to be MPBC. Patients who did not have a diagnosis of a second primary tumor in the ICR to 31 Dec. 2005 were designated as Single Primary Breast Cancer (SPBC) cases. The risk of MPBC relative to SPBC was determined by logistic regression, taking into account the number of years of follow up time from the first breast cancer diagnosis as a covariate. Age of onset comparisons were assessed by linear regression using NEMO software.

Family History Analysis: For each affected proband, a family history score was assigned using the Genealogical Database and ICR records. The circle of first to third degree relatives around the proband was determined by reference to the Genealogical Database. Then, the affection status of the relatives was determined from ICR records. Probands were assigned a family history score of 1 for each affected first degree relative, 0.5 for each affected second-degree relative, and 0.25 for each affected third-degree relative. The total score was then summed for each proband. Potential relationship between SNP marker genotype and family history score was then tested by Wilcoxon tests run on JMP v4 software (S.A.S. Institute Inc.).

Association Analysis Phenotypes: We used two related phenotypes for the association analysis. The first phenotype included all 1600 individuals diagnosed with breast cancer. This phenotype is designated “All Breast Cancer” (All BC). The second phenotype selected individuals with moderate to high predisposition characteristics and was designated “Medium Predisposition” (MedPre). The definition of this phenotype required that the proband fulfilled one of the following criteria:
1. The proband was a member of a cluster of breast cancer cases containing 3 or more affected relatives within a genetic distance of 3 meiotic events (3M).
2. The proband was a member of an affected pair related within 3M, one of whom was diagnosed when aged 50 or younger.
3. The proband was a member of an affected pair related within 3M, one of whom was diagnosed with a second primary tumor of any type.
4. The proband had been diagnosed with a second primary tumor of any type.

Out of the 1600 patients typed on the Illumina Hap300 chip, 653 fulfilled the MedPre criteria (40.8%).

### Results

Section 1: rs4848543 on Chromosome 2q14.2
The rs4848543 Allele A Allele is Associated with an Increased Risk for Breast Cancer:

Genome wide association analysis showed that rs4848543 allele A was associated with MedPre breast cancer. Illumina chip data for this SNP were obtained for 1598 patients (653 of whom fulfilled the MedPre phenotype criteria) and 4477 cancer-free controls. The RR estimate for rs4848543 allele A in association with MedPre breast cancer was 1.42 with a P-value of 8.3×10⁻⁸ when corrected for relatedness between individuals in the case and control groups (Table 1). Following Bonferroni correction for 317,089 SNPs tested, the overall corrected P-value was 0.026. The A allele of rs4848543 also showed nominally significant association with the AllBC phenotype, with a relative risk estimate of 1.16 (Table 1). In order to confirm this result in an independent sample from the Icelandic population, we designed and validated a Centaurus SNP assay, SG02S738, to test for the rs4848543 SNP. The SG02S738 assay was run on an additional 573 BC patients, of whom 198 fitted the MedPre phenotype definition. These patients were tested against an independent control group comprised of 7406 individuals who were typed for rs4848543 using the Illumina chip or the SG02S738 assay. The RR estimate for rs4848543 allele A in association with MedPre breast cancer in this second group was 1.26 (p-value=3.4×10⁻²). Therefore the original finding was replicated to nominal significance in an independent Icelandic sample. The results of a joint analysis, in which the original 1598 patients and the 573 replication patient set are combined and compared with 11883 controls is shown in Table 1. The joint analysis showed nominally significant risk for the AllBC phenotype, and genome-wide significant risk for the MedPre breast cancer phenotype. The overall Bonferroni corrected P-value for MedPre breast cancer was 0.0095 (correcting for 317,089 SNPs). This value is genome-wide significant, even if testing of the two phenotypes of AllBC and MedPre are taken into account.

Because the ICR records go back to 1955, some of the patients we recruited were long-term cancer survivors. If patients who carried the rs4848543 allele A variant had different probabilities of long-term survival than non-carriers, then the frequency of the variant amongst prevalent cases might be affected. To investigate this, we identified subset of patients composed of 833 individuals who were diagnosed after 1 Jan. 2000 and had times from diagnosis to recruitment of less than 5 years. In this cohort of recently diagnosed patients, the frequency of the rs4848543 allele A allele was 0.372945. We also identified a group of 1338 patients who had been diagnosed before 1 Jan. 2000 and had survived to recruitment. The frequency of the rs4848543 allele A amongst this group was 0.372749, which is not significantly different from the frequency amongst recently diagnosed patients (P value=0.99). Therefore there is no compelling evidence that differential survivorship affected the risk estimates shown in Table 1.

We noted additionally that breast cancer patients who did not have the MedPre phenotype did not display any increased frequency of rs4848543 allele A (Table 1). The implications of this observation are discussed further below. We went on to examine the relationships between rs4848543 allele A and various components of the MedPre phenotype definition:

rs4848543 Allele a is not Significantly Associated with Early Onset Breast Cancer:

Because a young age at diagnosis was one of the criteria used in the definition of the MedPre phenotype, we looked for an association between age at diagnosis and rs4848543 genotype. Amongst the 1598 patients who were analysed on the Illumina chips, there was no significant association between rs4848543 genotype and age at diagnosis (Table 2).

The Risk from rs4848543 Allele a is Associated with a Family History of Breast Cancer:

A family history score was calculated for each patient proband in the study. Using the Genealogical Database, we identified a circle of first- through third-degree relatives of each proband. Then, by reference to the ICR records, we identified all those relatives who had themselves been diagnosed with breast cancer. We then generated a summed family history score (FHS) for each proband, assigning a score of 1 for each affected first-degree relative, 0.5 for each affected second degree relative, and 0.25 for each third-degree relative. The 1598 probands who had been typed on the Illumina chip were then evaluated to look for an association between FHS and rs4848543 genotype. As shown in Table 2, a tendency for increased FHS was associated with carriage of the A allele of rs4848543. This result would not be expected based only on the observation that the rs4848543 allele A variant has a relative risk amongst AllBC patients of only 1.16; this relative risk alone could not generate a familial clustering of breast cancer cases [Stacey, et al., (2006), PLoS Med, 3, e217]. However, rs4848543 allele A was discovered using the MedPre phenotype, which has an substantial element of family history in its definition. These observations suggest that rs4848543 allele A may be in linkage disequilibrium with a less common, higher penetrance variant. Alternatively rs4848543 allele A might show interactions with another high penetrance predisposition determinants, increasing their penentrance and thereby the association with family history of breast cancer.

The Association Between Risk from rs4848543 Allele a and Family History of Breast Cancer is Partly (but not Completely) Explained by Families Carrying the BRCA2 999del5 Mutation:

In order to investigate further the relationship between rs4848543 allele A and family history of breast cancer, we asked whether families carrying the well characterized Icelandic BRCA2 999del5 mutation are responsible for the familial clustering exhibited by patients carrying rs4848543 allele A. We typed 1499 patients out of the set of 1600 for the BRCA2 999del5 mutation. We re-calculated the FHS as described above, this time excluding all probands who had been shown to carry the BRCA2 999del5 mutation. As shown in Table 2, the association between rs4848543 allele A and FHS was still significant after removal of the 999del5 mutation carriers, suggesting that the association cannot be fully explained by BRCA999del5 mutation families. However, the P-value was substantially higher than when 999del5 carriers had not been excluded, leading us to suspect that BRCA2 999del5 might be involved in some of the familial clustering exhibited by rs4848543 allele A.

The Risk from rs4848543 Allele a Extends to Carriers of the BRCA2 999del5 Mutation:

Since the BRCA2 999del5 mutation has such a substantial impact on familial breast cancer in Iceland, we considered its relationship with the rs4848543 allele A variant. Once possible scenario would be that the rs4848543 allele A variant confers negligible additional risk to BRCA2 999del5 carriers, as has been suggested for the interaction between CHEK2 and BRCA mutations [(2004), Am J Hum Genet, 74, 1175-82; Meijers-Heijboer, et al., (2002), Nat Genet, 31, 55-9]. If this were so, then the frequency of the rs4848543 allele A variant amongst affected BRCA2999del5 carriers would approximate the control frequency. Conversely, if the frequency of the rs4848543 allele A variant in affected BRCA2 999del5 carriers is greater than in the population controls, it would imply that the rs4848543 allele A confers risk on the BRCA2 carriers over and above the risk conferred by the 999del5 mutation. In order to maximize the number of BRCA2 999del5 carriers, the following analysis was carried out on the joint Icelandic Illumine and Replication patient sets. Examination of the data showed that the frequency of the rs4848543 allele A variant was 0.463 in BRCA2 999del5 carriers with breast cancer, significantly greater than the control frequency and corresponding to an RR of 1.65 (Table 3). Therefore, BRCA2 999del5 carriers (who are already at a high risk of breast cancer) have their risk multiplied by an estimated factor of 1.65 for each rs4848543 allele A allele carried. There was no evidence for a deviation of the rs4848543 genotypes from the multiplicative model. These observations demonstrate that the increased risk of breast cancer conferred by the rs4848543 allele A variant extends to BRCA2 999del5 carriers. Since BRCA2 999del5 is a null mutation that produces a non-functional protein, these results suggest that the risk confers by rs4848543 allele A extends to all carriers of BRCA2 mutations where the mutation renders the protein non-functional.

rs4848543 Allele a May Interact Synergistically with BRCA2 999del5:

The observation that the risk from rs4848543 allele A extends to BRCA2 999del5 carriers raises the question of whether the variant confers the same relative risk upon 999del5 carriers as it does upon 999del5 non-carriers. We noted that the relative risk estimate for rs4848543 allele A in a BRCA2 999del5 carrier (1.65) was somewhat higher than the relative risk estimate for rs4848543 allele A in non-carriers of the BRCA2 mutation (1.13, Table 3). We therefore tested whether the relative risk conferred by rs4848543 allele A was different in a BRCA2 999del5 background than it was in a non-999del5 background. As shown in Table 3, the relative risk conferred by rs4848543 allele A was an estimated 1.46-fold higher in 999del5 carriers than in non-carriers. These observations are consistent with the interpretation that rs4848543 allele A interacts in a synergistic manner with BRCA2 999del5. Since BRCA2 999del5 is a null mutation that produces a non-functional protein, these results suggest that rs4848543 allele A interacts in a synergistic manner with all BRCA2 mutations where the mutation renders the protein non-functional.

The potential interaction with BRCA2 999del5 was also investigated by evaluating whether, amongst patients, there was a significant correlation between numbers of variant alleles of rs4848543 and 999del5 carried by patients. As shown in Table 4, there was, in patients, a significant correlation between the number of rs4848543 allele A alleles carried and the carriage of BRCA2 999del5 alleles. In order to investigate whether the correlation between rs4848543 allele A and BRCA2 999del5 alleles in patients might have arisen due to an underlying population stratification, we examined whether the correlation existed in the control population. There was no correlation amongst 5938 controls who had been tested for both variants. Breast cancer patients were excluded from this group of 5938 controls. Because BRCA2 999del5 is such-a strong risk factor for breast cancer, it might be argued that the control group was depleted of 999del5 carriers (since relatively few female carriers might be unaffected with breast cancer). We therefore repeated the test using a control group comprised of 2925 male subjects who had been tested for both variants. There was no evidence of a correlation between rs4848543 allele A and 999del5 carriage in this group. Therefore we concluded that there is no evidence of an underlying population stratification that might account for the co-inheritance of rs4848543 allele A and 999del5 amongst patients. Accordingly, the most likely explanation is that rs4848543 allele A and 999del 5 interact in an synergistic manner.

We then returned to our observation that rs4848543 allele A shows an association with family history when known BRCA2 999del5 patients are removed from the analysis (Table 2). We asked whether high risk patients who were not BRCA2 999del5 carriers but who fitted the MedPre phenotype criteria demonstrated an increased frequency of rs4848543 allele A. The frequency was higher in these patients than in controls, corresponding to a relative risk of 1.35 (Table 3).

Thus, the risk association with rs4848543 allele A extends to patients who are not 999del5 carriers but who nevertheless have the MedPre characteristics.

As shown in Table 1, there was no detectable risk associated with rs4848543 allele A in patients who do not fit the MedPre phenotype definition. This suggests that rs4848543 allele A only confers risk in the context of a MedPre phenotype background. This could occur if a relatively rare, high penetrance mutation exists on the background of the rs4848543 allele A variant. Alternatively, the rs4848543 allele A variant could be indicative of a more common allele that modifies the penetrance of BRCA2 and other, as yet unidentified, high penetrance risk determinants that have a familial association. Such unidentified risk determinants might be high penetrance variants of other genes, polygenic combinations of variants, or even environmental risk factors that cluster in families. For convenience, we refer to such hypothetical high penetrance risk determinants as BRCAx. To further investigate the notion that rs4848543 allele A might act as a modifier gene, we asked whether rs4848543 allele A occurred in the same frequency amongst affected BRCA2 999del5 carriers as it does on MedPre affected, non-BRCA carriers. This, in effect, queries whether the rs4848543 allele A variant confers a similar risk on carriers of 999del5 as it does on individuals with BRCAx. As shown in Table 3, there was no significant difference in frequencies of the rs4848543 allele A variant between these groups. However the point estimate of the relative risk was 1.22, suggesting that the effect might be greater in 999del5 carriers.

Carriers of rs4848543 Allele a and a Number of Other Markers within the rs4848543 LD Block (in Linkage Disequilibrium with rs4848543) are at Increased Risk for Multiple Primary Breast Cancer:

Occurrence of multiple primary breast tumors is an indicator of hereditary predisposition and has clinical relevance. Patients who have had breast cancer are at greatly increased risk for developing a second primary tumor. Genetic predisposition may form a substantial component of this increased risk. We therefore investigated whether multiple primary breast cancer (MPBC) occurred at higher than expected frequencies in rs4848543 allele A carriers than non-carriers. Data from the Illumina chip indicated that other markers in the rs4848543 LD block might predict MPBC at least as well as rs4848543. We therefore designed and tested a battery of 8 assays for markers within the block. In an evaluation of the risk for MPBC using logistic regression and correcting for the length of patients' follow-up times, 7 out of 8 SNPs showed an increased risk of MPBC in carriers when compared with patients who had only experienced a single primary breast cancer (SPBC) diagnosis (Table 5). We concluded that these 7 SNP assays can determine whether a patient is at an increased risk of MPBC.

Multiple SNPs in One Linkage Disequilibrium Block Show Associations with all BC and MedPre Breast Cancer Phenotypes:

The SNP rs4848543 allele A is found in a linkage disequilibrium (LD) block whose delimiting coordinates, based on NCBI Build 34, are shown in Table 6. The LD block was defined as extending between recombination hotspots as defined by the Mathematical Genetics Group at the University of Oxford using the likelihood ratio test described in [McVean, et al., (2004), Science, 304, 581-4; Winckler, et al., (2005), Science, 308, 107-11]. Data from the Illumina Hap300 chip revealed a number of SNPs that are in the same linkage disequilibrium block as rs4848543 allele A and that showed nominally significant associations with the two breast cancer predispositions tested. Tables 7 & 8 show a lists of all of the SNPs in the LD block that are present on the Illumina Hap300 chip, and the association values obtained from them for the two phenotypes. We also considered two-marker haplotypes within the block that were identified as efficient surrogates (r²>0.8) for an additional set of SNPs typed in the HapMap project (all references to HapMap data are with respect to release 20). These additional SNPs have a minor allele frequency of >5% in the UTAH CEPH (CEU) HapMap samples and they are neither on the Hap300 SNP chip nor are they efficiently tagged by SNPs that are on the chip [Pe'er, et al., (2006), Nat Genet, 38, 663-7]. Results for these so-called “Daly” haplotypes are also shown in Tables 7 & 8. For MedPre breast cancer, 29 individual SNPs or two-point “Daly” haplotypes showed nominally significant p-values.

rs4848543 Allele A-Associated Risk is Present on Multiple Haplotype Backgrounds.

In order to try to differentiate whether rs4848543 allele A tags a relatively rare, high penetrance variant or behaves as a common modifier of other high penetrance determinants, we investigated the diversity of haplotype backgrounds in which rs4848543 allele A appears. The rs4848543 LD block contains a region of relatively high recombination, with its distal end at around nucleotide 120034174. The SNPs that confer nominally significant breast cancer risk are located distal (rightwards) to this region of recombination. We therefore limited our analysis of the haplotype diversity to this right-hand sub-block region. We used NEMO software to identify haplotypes within the defined region (nt 120034174 to nt 120129001) using SNPs and samples that were typed on the Illumina chips. Redundant SNPs that were not necessary to differentiate haplotypes were eliminated, then relative risks and frequencies were evaluated for each resulting haplotype. The results are shown in Table 9. It is apparent that all identified haplotypes that carry the rs4848543 A allele had a relative risk estimate greater than 1 and, in most cases the RR estimate was significantly greater than 1. Conversely, no haplotype that carried the rs4848543 C allele had a RR estimate greater than 1, and all the significant estimates were less than 1 (i.e. they were protective). Thus we were unable to separate the risk from rs4848543 allele A-bearing haplotypes. This suggests that the pathogenic variant (i.e. the variant that, mechanistically, confers risk) is rs4848543 allele A or a variant in strong linkage disequilibrium with rs4848543 allele A. The presence of the pathogenic variant on multiple haplotype backgrounds suggests that its frequency might closely approximate that of rs4848543 allele A (i.e. the r² value between rs4848543 allele A and the pathogenic variant is high). This observation favors the notion that rs4848543 allele A does not indicate the presence of a rare, high penetrance variant within the rs4848543 LD block, but rather it marks a rather common modifier of high penetrance determinants such as BRCA2 and the previously defined BRCAx.

Numerous Markers could Show BC Risk Association Through their Correlation with rs4848543 Allele A:

Reference to the HapMap project data (release 20) revealed a number of known SNPs that are correlated with rs4848543 with r² values greater than 0.2 in the CEU white European population sample. Since these SNPs are significantly correlated with rs4848543, they could be used to measure the same BC risk as is measured by rs4848543 itself. These SNPs are listed in Table 10.

### The STEAP3/TSAP6 Gene is the Most Probable Gene Involved in the Observed Risk for Breast Cancer:

There are three presently known genes in the rs4848543 LD block region. These are;
1. The Complement Component 1q Subcomponent-like 2 gene C1QI2 (NM_{—}182528)
2. The Six Transmembrane Epithelial Antigen of Prostate 3 (STEAP3) gene, otherwise known as Tumour Suppressor Activated Pathway 6 (TSAP6), pHyde, and Dudulin-2. There are three major known alternative transcripts of this gene identified by RefSeq numbers NM_{—}018234 (isoform b), NM_{—}182915 (isoform a), and NM_{—}001008410 (also designated AF262322). A fourth transcript of this gene has been described and assigned the identifier AK024163.
3. A transcript, AK127773, overlaps with the STEAP3 gene and is transcribed from the opposite (leftwards) strand. This gene has been designated Hypothetical protein F1145874.

The rs4848543 LD block contains a region of relatively high recombination, with its distal end at around nucleotide 120034174. The majority of SNPs and Daly 2-point haplotypes that confer nominally significant breast cancer risk are located distal (rightwards) to this region of recombination. This suggests that variants in and around C1QI2 are not the source of the association signal. The variants that do associate strongly with risk are located 5′ to and in the STEAP3/TSAP6 gene. Indeed two of the nominally significant SNPs, rs838100 and rs3731603 are within the transcribed sequences of the gene and another two, rs838102 and rs12711924 are located in STEAP3/TSAP6 introns. The latter of these two SNPs is located near the 3′ splice site of the IVS4 gene. Given these observations, and the known biology of the STEAP3/TSAP6 gene (see below), the most likely gene to be involved in the observed increased risks for breast cancer is STEAP3/TSAP6.

Section 2: rs13387042 on Chromosome 2q35
The rs13387042 A Allele is Associated with an Increased Risk for Breast Cancer in Iceland:

Genome-wide association analysis showed that rs13387042 allele A was associated with the Any BC phenotype (Table 11). This SNP is located at chromosome 2q35, at a distinct location from the STEAP3/TSAP6 locus. Illumina chip data for this SNP were obtained for 1598 patients and 4475 cancer-free controls. The RR estimate for A-rs13387042 was 1.19 with a P-value of 4.0×10⁻⁵ when corrected for relatedness between individuals in the case and control groups. The RR was similar in the MedPre breast cancer, with a P-value of 4.4×10⁻³.

In itself, this result did not reach a level that would be considered genome-wide significant after Bonferroni correction for the 317,089 SNPs tested. We therefore sought to confirm the result in an independent sample from the Icelandic population, as we did for rs4848543 allele A. We designed and tested a Centaurus SNP assay, designated SG02S728, to test for the rs13387042 SNP. The SG02S728 assay was run on an additional 583 patients and an independent control group comprised of 7966 individuals who were typed for rs13387042 allele A, either by the Illumina chip or by the Centaurus methods. The 7966 control group contained individuals who had been diagnosed with prostate cancer or colorectal cancer but not individuals who had been diagnosed with breast cancer. This was justified by our interim observations that rs13387042 allele A did not confer detectable risk for these cancers (data not presented). The RR estimate for the A allele of rs13387042 in association with AnyBC in this second group was 1.20 (P-value_{corr}=3.8×10⁻³). Therefore the original finding was replicated significantly in an independent Icelandic sample, with a very similar point estimate of the RR. The results of a joint analysis, in which the original 1598 and the 583 replication samples are combined and compared with 12441 controls is shown in Table 11. The joint analysis showed a significant relative risk estimate of 1.20 (P-value_{corr}=2.0×10⁻⁷), which is close to the level of significance after Bonferroni correction for the 317,089 SNPs tested. The observed frequency and relative risk of the rs13387042 allele A variant corresponds to an estimated population attributable risk of 15.6% in the Icelandic population.

Because the ICR records go back to 1955, some of the patients we recruited were long-term cancer survivors. If patients who carried the rs13387042 allele A variant had different probabilities of long-term survival than non-carriers, then the frequency of the variant amongst prevalent cases might be affected. To investigate this, we identified subset of patients composed of 837 individuals who were diagnosed after 1 Jan. 2000 and had times from diagnosis to recruitment of less than 5 years. In this cohort of recently diagnosed patients, the frequency of rs13387042 allele A was 0.498. We also identified a group of 1344 patients who had been diagnosed before 1 Jan. 2000 and had survived to recruitment. The frequency of rs13387042 allele A amongst this group was 0.492, which is not significantly different from the frequency amongst recently diagnosed patients (P value=0.71). Therefore there is no compelling evidence that differential survivorship affected the risk estimates shown in Table 11.

The rs13387042 A Allele is Associated with an Increased Risk for Breast Cancer in a Spanish Case: Control Sample

In order to further investigate the role of rs13387042 allele A in breast cancer risk, we examined an independent set of BC case:control samples collected in Spain. The Spanish study population consisted of 446 breast cancer cases recruited from the Oncology Department of Zaragoza Hospital in Zaragoza, Spain. A set of 977 control individuals were approached at the University Hospital in Zaragoza, Spain, and were confirmed to be free of breast cancer before they were included in the study. All subjects gave written informed consent.

The Spanish case and control samples were genotyped for rs13387042 using the SG02S728 Centaurus assay. The results are presented in Table 11. Allele A of rs13387042 showed significant association with Breast Cancer risk in this population, with a point estimate of the RR of 1.21 (P-value 1.8×10⁻²). The frequency of the rs13387042 allele A variant was higher in the Spanish control samples, suggesting that the variant may be more prevalent and thus contribute to a higher Breast Cancer burden in populations of Spanish extraction.

The rs13387042 A Allele is Associated with an Increased Risk for Breast Cancer in Swedish Case/Control Samples:

To seek further confirmation of the role of the A allele of the rs13387042 variant in risk for Breast Cancer, we typed the SNP in two Swedish cohorts: The “Sweden Familial” cohort was comprised of 346 BC patients who had been referred to the oncogenetic counseling clinic of the Karolinska Institute, Stockholm for investigation of a family history of breast cancer. Each patient came from a distinct family. The “Sweden Consecutive” cohort was comprised of 482 consecutively recruited BC patients who attended the Karolinska Institute breast cancer clinic. Family history was not taken into account in the selection of this second cohort. Controls were 1300 blood donors from the Stockholm area and 434 cancer-free spouses of colorectal cancer cases. There was no significant difference between genders in the frequency of A-rs13387042 in the controls, nor was there a significant difference between the blood donor and cancer-free control sets.

As shown in Table 11, the “Sweden Consecutive” cohort demonstrated a significant relative risk for breast cancer of 1.31 (P-value=2.0×10⁻⁴). The “Sweden Familial” cohort relative risk estimate of 1.11 was not significant, perhaps because this group may carry high-penetrance genes that overshadow the effect of A-rs13387042. Overall, the combined Swedish cohorts returned a significant relative risk estimate of 1.22 (P-value=8.1×10⁻⁴).

The estimates for the Icelandic, Spanish and Swedish cohorts were combined in a joint analysis, using the Mantel-Haenszel model. The result was an estimated relative risk of 1.20 with a P-value of 3.8×10⁻¹¹. This is well below the threshold for genome-wide significance using the Bonferroni method to correct for the 317,089 SNPs investigated. We therefore concluded that A-rs13387042 confers a significant and reproducible risk for breast cancer in several population samples of European descent. The overall population attributable risk was estimated to be 16.4%.

rs13387042 Allele a—Associated Risk is Present on Multiple Haplotype Backgrounds:

The rs13387042 SNP is found in a linkage disequilibrium (LD) block whose delimiting coordinates, based on NCBI Build 34, are shown in Table 12. The LD block was defined as extending between recombination hotspots as defined by the Mathematical Genetics Group at the University of Oxford using the likelihood ratio test described in [McVean, et al., (2004), Science, 304, 581-4; Winckler, et al., (2005), Science, 308, 107-11]. In order to try to differentiate whether the A allele of marker rs13387042 tags a relatively rare, high penetrance variant or a more common, lower penetrance variant we investigated the diversity of haplotype backgrounds in which rs13387042 allele A appears. We used NEMO software to identify haplotypes within the LD block using NSPs and samples that were typed on the Illumine chips. Redundant SNPs that were not necessary to differentiate haplotypes were eliminated, then relative risks and frequencies were evaluated for each resulting haplotype. The results are shown in Table 13. It is apparent that several identified haplotypes that carry the A allele of rs13387042 allele confer relative risk estimates greater than 1 and, in several cases the RR estimate was significantly greater than 1. Conversely, only one, rare haplotype that carried the C allele of rs13387042 allele showed a RR estimate greater than 1, and the two nominally significant RR estimates for rs13387042 allele C-containing haplotypes were less than 1 (i.e. they were protective). Two haplotypes that carry the A allele of rs13387042 did not produce RR estimates greater than 1, and one of these estimates was nominally significant. Taken together, these observations suggests that the pathogenic variant (i.e. the variant that, mechanistically, confers risk) is in strong LD with rs13387042 allele A but may not be rs13387042 allele A itself.

Multiple SNPs in the Rs13387042 LD Block Show Association with BC Risk:

Data from the Illumina Hap300 chip revealed a number of SNPs that are in the same LD block as rs13387042. Table 14 shows a list of all the SNPs in the LD block that are present on the Illumina Hap300 chip and the association values obtained from them for single point and two point “Daly” haplotype analysis. It can be seen that 21 SNPs or Daly haplotypes showed nominally significant P-values when corrected for familial relationships amongst the subjects.

Numerous Markers could Show BC Risk Association Through their Correlation with rs13387042 Allele A:

Reference to the HapMap project data (release 20) revealed a number of known SNPs that are correlated with rs13387042 with r² values greater than 0.2 in the CEU white European population sample. Since these SNPs are significantly correlated with rs13387042, they could be used to measure the same BC risk as is measured by rs13387042 itself. These SNPs are listed in Table 15.

Section 3: rs3803662 on Chromosome 16q12
The T Allele of rs3803662 is Associated with Risk for Breast Cancer:

Further genome wide SNP analysis using data from 1600 breast cancer patients and 11563 controls identified the T allele of SNP rs3803662 as conferring and estimated 1.23-fold increased risk for Any BC (Table 16). This result was confirmed in a second, independent cohort of 594 Icelandic breast cancer patients and 1433 controls. The combined data from these two Icelandic samples gave a relative risk estimate of 1.23 and a P-value of 2.8×10⁻⁷ when corrected for relatedness between the individuals. This corresponded to an estimated population attributable risk of 10.1% (Table 16).

Association rs3803662 Allele T with Risk for Breast Cancer is Replicated in Independent Breast Cancer Case: Control Cohorts from Sweden, Spain, and Holland.

In order to confirm the association of rs3803662 allele T with breast cancer risk, we sought to replicate the finding in three independent, foreign cohorts. We genotyped individuals from the Swedish and Spanish cohorts described above. We also genotyped 558 breast cancer cases and 1384 controls from Nijmegen, Holland. This cohort was drawn from a population-based registry ascertainment of breast cancer patients diagnosed in the Eastern part of the Netherlands in the years 2005-2006. All patients diagnosed with breast cancer before the age of 70 years during this period were invited to participate in the study. The control group was collected in a survey conducted in 2002-2003 (the Nijmengen Biomedical Study) based on a random sample of the population registration. As shown in Table 16, a significantly increased risk for rs3803662 allele T was observed in all three of these replication cohorts. In a combined analysis of these three non-Icelandic replication cohorts, the overall relative risk estimate was 1.35 and the P-value was 5.1×10⁻¹². A joint analysis of the Icelandic and replication cohorts indicated a combined relative risk estimate of 1.28 with a P-value of 2.7×10⁻¹⁷. This is well below the threshold for genome-wide significance when corrected for the number of SNPs tested. The corresponding overall population attributable risk estimate was 13.4% (Table 16).

Definition of the LD Block Containing rs3803662 and Identification of Potential Candidate Genes.

The rs3803662 SNP is found in a linkage disequilibrium (LD) block whose delimiting coordinates, based on NCBI Build 34, are shown in Table 17. The LD block was defined as extending between recombination hotspots as defined by the Mathematical Genetics Group at the University of Oxford using the likelihood ratio test described in [McVean, et al., (2004), Science, 304, 581-4; Winckler, et al., (2005), Science, 308, 107-11]. The rs3803662 is present in the transcript of a hypothetical protein designated LOC643714. This hypothetical protein is thus implicated as a potential cancer predisposition gene. The 5′ end of known gene TNRC9 is also present in the block. TNRC9 is a member of the chromatin associated high mobility group (HMG) protein family, a group of proteins which is known to include transcription factors and chromatin remodeling agents. Reduced expression of the TNRC9 protein has been observed in metastatic breast cancer when compared to primary breast cancers (Olendrowitz, C. 2006, Bachelor of Science Bioinformatics Thesis, Charité-Universitatsmedizin, Berlin). The identification of a SNP variant near the 5′ end of this gene suggests that the variant may be in linkage disequilibrium with genetic factors that alter the expression of function of the TNRC9 protein, leading to an increased breast cancer risk.

Multiple SNPs in the rs3803662 LD Block Show Association with BC Risk:

Data from the Illumina Hap300 chip revealed a number of SNPs that are in the same LD block as rs3803662. Table 18 shows a list of all the SNPs in the LD block that are present on the Illumina Hap300 chip and the association values obtained from them for single point and two point “Daly” haplotype analysis. It can be seen that 80 SNPs or Daly haplotypes showed nominally significant P-values.

Numerous Markers could Show Bc Risk Association Through their Correlation with rs3803662 Allele T:

Reference to the HapMap project data (release 20) revealed a number of known SNPs that are correlated with rs3803662 with r² values greater than 0.2 in the CEU white European population sample. Since these SNPs are significantly correlated with rs3803662, they could be used to measure the same BC risk as is measured by rs3803662 itself. These SNPs are listed in Table 19.

rs3803662 Allele T and/or Related SNPs Confer Risk for Breast Cancer in Several Different Ethnicities:

In order to investigate the generality of the findings with rs3803662 allele T in patients from different ethnic background, we tested the SNP for association in a sample of breast cancer patients and controls from the Multiethnic Cohort study (MEC). The MEC consists of over 215,000 men and women in Hawaii and Los Angeles (with additional African-Americans from elsewhere in California) and has been described in detail elsewhere [Kolonel, et al., (2000), Am J Epidemiol, 151, 346-57]. The cohort is comprised predominantly of African Americans, Native Hawaiians, Japanese Americans, Latinos, and European Americans who entered the study between 1993 and 1996 by completing a 26-page self-administered questionnaire that asked detailed information about dietary habits, demographic factors, personal behavior, history of prior medical conditions, family history of common cancers, and for women, reproductive history and exogenous hormone use. The participants were between the ages of 45 and 75 at enrolment. Incident cancers in the MEC were identified by cohort linkage to population-based cancer Surveillance, Epidemiology, and End Results (SEER) registries covering Hawaii and Los Angeles County and to the California State Cancer Registry covering all of California. Beginning in 1994, blood samples were collected from incident breast cancer cases and a random sample of MEC participants to serve as a control pool for genetic analysis in the cohort. Eligible cases in the nested breast cancer case-control study consisted of women with incident invasive cancer diagnosed after enrolment into the MEC through Dec. 31, 2002. Controls were participants without breast cancer, respectively, prior to entry into the cohort and without a diagnosis up to Dec. 31, 2002. Controls were frequency matched to case based on race/ethnicity and age (in 5-year intervals). The study was approved by the Institutional Review Boards at the University of Southern California and at the University of Hawaii.

We sought to investigate the risk associated with rs3803662 in European Americans, Latina, and African Americans from the MEC. Because the correlations between the rs13387042 SNP and other SNPs might be different in different ethnicities, we selected a group of SNPs to test in these samples. The SNPs, their r² and D values derived from HapMap project data (release 20) for European Americans and Yoruba (Africans) are shown in Table 23. Centaurus assays were generated for these SNPs, and genotyping was carried out in the MEC samples. To be better able to compare results between SNPs, we used the generalized likelihood ratio test taking into account missing genotypes by haplotype analysis. This had the effect of equalizing the number of cases and controls for different SNPs, allowing for an easier comparison between them. Results are shown in Table 24. Firstly it is evident that the risk from rs3803662 allele T is replicated significantly in European Americans. Two of the related SNPs, rs4784227 allele T and rs17271951 allele C show nominally higher relative risks and lower P-values than rs3803662 allele T in European Americans. This exemplifies the concept that related to the SNPs originally identified might have higher relative risks and thus provide superior disease risk markers.

The rs3803662 allele T signal is also replicated significantly in Latina. The T allele of marker rs3803662 did not replicate significantly in African Americans, indeed the T-allele was significantly protective in the African American sample. This suggests the LD relation between the rs3803662 allele T and the putative pathogenic mutation is quite different in African Americans. We noted that the three related SNP alleles; rs12922061 allele T, rs4784227 allele T, and rs17271951 allele C are strongly correlated with rs3803662 allele T in Europeans, but this correlation is not maintained in Yoruban Africans (Table 24). However, unlike rs3803662 allele T, the related SNP alleles rs12922061 allele T, rs4784227 allele T, and rs17271951 allele C all show nominally increased relative risks for breast cancer, two of which achieve statistical significance at the 1-sided level (Table 24). This shows that using sets of related SNPs can provide risk markers suitable for use in a variety of ethnic groups.

rs13387042 Allele a and Rs3803662 Allele T Confer Increased Risk for Estrogen Receptor Positive Breast Cancer:

Medical records of patients in the study were reviewed if they were available. Using the combined sample sets, we looked for associations between rs13387042 allele A and rs3803662 allele T, age at diagnosis, estrogen receptor (ER) and progesterone receptor (PR) status. Neither variant showed an association with age at diagnosis. Significant breast cancer risk involving rs13387042 allele A and rs3803662 allele T was clearly confined to those patients diagnosed with ER positive tumors and the difference between the OR's for ER positive and ER negative tumors was significant (Table 24). Similarly, there was a tendency towards breast cancer risk preferentially amongst patients diagnosed with PR positive tumors, however the difference between PR positive and PR negative OR's was not significant. This suggests that ER positive and negative tumors may have somewhat different genetic etiologies.

### Tables:

TABLE 1
Association of SNP SG02S738 (rs4848543) allele A with risk for breast
cancer:

| | p-val | p_corr* | r | 95% CI | #aff | aff. freq | #con | con. freq |
|---|---|---|---|---|---|---|---|---|
| MedPre Illumina | 6.70E−09 | 8.30E−08 | 1.42 | (1.25, 1.62) | 653 | 0.423 | 4477 | 0.341 |
| MedPre Iceland Replication | 2.90E−02 | 3.40E−02 | 1.26 | (1.02, 1.55) | 198 | 0.399 | 7406 | 0.346 |
| MedPre joint | 9.70E−10 | 3.00E−08 | 1.37 | (1.23, 1.53) | 851 | 0.418 | 11883 | 0.344 |
| All Illumina | 7.10E−04 | 1.10E−03 | 1.16 | (1.06, 1.26) | 1598 | 0.374 | 4477 | 0.341 |
| All Iceland Replication | 9.70E−02 | 1.00E−01 | 1.11 | (0.98, 1.26) | 573 | 0.37 | 7406 | 0.346 |
| All joint | 2.20E−04 | 3.50E−04 | 1.14 | (1.06, 1.22) | 2171 | 0.373 | 11883 | 0.344 |
| non-MedPre vs. CTR | 9.80E−01 | | 1 | | 1320 | 0.344 | 11883 | 0.344 |

*corrected for relationships between individuals

TABLE 2
Association of SNP SG02S738 (rs4848543) with age
at diagnosis and family history of breast cancer:

| | P-value |
|---|---|
| Comparison | (Wilcoxon) |
| rs4848543 Genotype vs Age at Diagnosis | 0.5139 |
| rs4848543 Genotype vs Family History Score | <0.001 |
| Non-BRCA2 rs4848543 Genotype vs Family History | 0.0044 |
| Score | |

TABLE 3
Association of SNP SG02S738 (rs4848543) with risk for breast
cancer amongst carriers of BRCA2 999del5:

| | p_corr* | p-val | r | 95% CI | #aff | aff. freq | #con | con. freq |
|---|---|---|---|---|---|---|---|---|
| BC BRCA2 carr vs. | 3.67E−03 | 8.02E−04 | 1.65 | (1.18, 2.30) | 94 | 0.463 | 9452 | 0.344 |
| CTR | | | | | | | | |
| BC BRCA2 non carr | 2.95E−03 | 2.19E−03 | 1.13 | (1.04, 1.22) | 1621 | 0.371 | 9452 | 0.344 |
| vs. CTR | | | | | | | | |
| BC BRCA2 carr vs. BC | 3.06E−02 | 1.29E−02 | 1.46 | (1.04, 2.05) | 94 | 0.463 | 1621 | 0.371 |
| BRCA2 non car | | | | | | | | |
| MedPre BRCA2 non- | 4.71E−06 | 5.92E−07 | 1.35 | (1.19, 1.55) | 622 | 0.414 | 9452 | 0.344 |
| carr vs. CTR | | | | | | | | |
| BC BRCA2 carr vs | n.a. | 2.08E−01 | 1.22 | n.a. | 94 | 0.463 | 622 | 0.414 |
| MedPre BRCA2 non- | | | | | | | | |
| carr | | | | | | | | |

*corrected for relationships between individuals

TABLE 4
Correlation between rs4848543 allele A and BRCA2 999del5 Genotypes:
BRCA2 - STEAP3 genotype correlation
(Pearson test)

| | P-val | corr. coeff. | #indiv |
|---|---|---|---|
| BC patients | 1.4E−02 | 0.059 | 1715 |
| controls | 8.0E−01 | −0.0033 | 5938 |
| male controls | 6.7E−01 | −0.0079 | 2925 |

TABLE 5
Association of SNPs with risk for multiple primary breast cancer:

| #MPBC | MPBC. freq | #SPBC | SPBC. freq | P carrier | RR carrier | Ale Mrk | rs SNP | Pos in Seq ID 4 |
|---|---|---|---|---|---|---|---|---|
| 128 | 0.258 | 1566 | 0.189 | 0.0111 | 1.615 | 4 SG02S733 | rs895398 | 34898 |
| 160 | 0.409 | 2010 | 0.370 | 0.0433 | 1.419 | 1 SG02S738 | rs4848543 | 37814 |
| 160 | 0.547 | 2019 | 0.488 | 0.0438 | 1.498 | 1 SG02S739 | rs6759589 | 41694 |
| 163 | 0.414 | 2001 | 0.371 | 0.0389 | 1.427 | 4 SG02S753 | rs895397 | 49118 |
| 162 | 0.645 | 2020 | 0.575 | 0.0072 | 1.928 | 3 SG02S740 | rs838102 | 56596 |
| 160 | 0.650 | 2014 | 0.575 | 0.0050 | 2.012 | 3 SG02S741 | rs838100 | 60428 |
| 127 | 0.445 | 1565 | 0.380 | 0.0176 | 1.602 | 1 SG02S734 | rs12711924 | 84034 |
| 159 | 0.387 | 2004 | 0.343 | 0.1741 | 1.258 | 4 SG02S742 | rs3731603 | 93780 |

TABLE 6
Definition of the rs4848543 LD block on Chromosome 2:

| NCBI Build | Start | End |
|---|---|---|
| 34 | 119,987,002 | 120,129,001 |
| 36 | 119,608,327 | 119,750,326 |

TABLE 7
Association of SNP markers and 2-marker haplotypes in the rs4848543 LD-
block with risk for breast cancer (phenotype Any Breast Cancer):
The allele codes are as follows: 1 = A, 2 = C, 3 = G, 4 = T.

| p-value | RR | #aff | aff. freq | #con | con. freq | H0. freq | X2 | info_alt | all | marker | all | marker |
|---|---|---|---|---|---|---|---|---|---|---|---|---|
| 0.0000825 | 1.1852 | 1557 | 0.377649 | 4626 | 0.33863 | 0.348455 | 15.5001 | 1 | 1 | rs4848543 | | |
| 0.00015 | 0.8545 | 1558 | 0.498176 | 4627 | 0.537411 | 0.527529 | 14.3718 | 0.999171 | 2 | rs895398 | 3 | rs6759589 |
| 0.0001842 | 1.1677 | 1558 | 0.500642 | 4626 | 0.461954 | 0.471701 | 13.9859 | 1 | 1 | rs6759589 | | |
| 0.0004919 | 1.2131 | 1558 | 0.18702 | 4627 | 0.159399 | 0.166396 | 12.1462 | 0.966913 | 2 | rs4849765 | 4 | rs895398 |
| 0.0006022 | 1.1539 | 1558 | 0.560738 | 4627 | 0.525225 | 0.534172 | 11.7693 | 0.994181 | 2 | rs4849760 | 3 | rs838102 |
| 0.0006816 | 1.1529 | 1558 | 0.587612 | 4625 | 0.552757 | 0.56154 | 11.5388 | 1 | 3 | rs838102 | | |
| 0.0007483 | 1.1517 | 1556 | 0.587082 | 4622 | 0.552466 | 0.561185 | 11.3653 | 1 | 3 | rs838100 | | |
| 0.0007856 | 0.8681 | 1558 | 0.401958 | 4627 | 0.436379 | 0.427706 | 11.2749 | 0.995483 | 3 | rs6759589 | 1 | rs838102 |
| 0.0009395 | 1.1929 | 1557 | 0.196853 | 4626 | 0.17045 | 0.177098 | 10.9433 | 1 | 4 | rs895398 | | |
| 0.0009835 | 1.1959 | 1556 | 0.204802 | 4619 | 0.177192 | 0.184711 | 10.8584 | 0.936107 | 2 | rs4849765 | 4 | rs2084154 |
| 0.0019878 | 1.1829 | 1558 | 0.187099 | 4626 | 0.162884 | 0.168984 | 9.5608 | 1 | 4 | rs935361 | | |
| 0.0039725 | 1.3609 | 1558 | 0.067214 | 4627 | 0.050286 | 0.054483 | 8.2963 | 0.668984 | 2 | rs838086 | 1 | rs12711924 |
| 0.0044063 | 1.1296 | 1558 | 0.387356 | 4627 | 0.358872 | 0.366047 | 8.1083 | 1 | 1 | rs12711924 | | |
| 0.0053777 | 1.1574 | 1558 | 0.202503 | 4627 | 0.179922 | 0.18561 | 7.7478 | 1 | 2 | rs7564432 | 2 | rs11680207 |
| 0.0076689 | 1.1238 | 1558 | 0.349807 | 4627 | 0.323752 | 0.330315 | 7.1092 | 1 | 4 | rs3731603 | | |
| 0.0092636 | 0.895 | 1558 | 0.58833 | 4626 | 0.614901 | 0.608199 | 6.7713 | 0.984119 | 2 | rs6542504 | 2 | rs12711923 |
| 0.0119574 | 1.1118 | 1557 | 0.418433 | 4626 | 0.392888 | 0.399321 | 6.3172 | 1 | 4 | rs1530561 | | |
| 0.0120093 | 1.1289 | 1558 | 0.253591 | 4627 | 0.231338 | 0.236949 | 6.3095 | 0.998425 | 1 | rs745888 | 2 | rs838087 |
| 0.0127242 | 1.1276 | 1558 | 0.253851 | 4625 | 0.231784 | 0.237344 | 6.2071 | 1 | 1 | rs745888 | | |
| 0.0140734 | 0.8975 | 1554 | 0.330746 | 4621 | 0.355095 | 0.348951 | 6.0289 | 0.987772 | 3 | rs2084154 | 4 | rs838066 |
| 0.0143097 | 0.8986 | 1558 | 0.337519 | 4627 | 0.361823 | 0.355701 | 5.9995 | 0.993263 | 3 | rs6759589 | 4 | rs838066 |
| 0.0158735 | 0.9024 | 1558 | 0.586151 | 4627 | 0.610816 | 0.60459 | 5.8168 | 0.983893 | 2 | rs6542504 | 2 | rs4849760 |
| 0.0166389 | 1.1057 | 1558 | 0.432285 | 4627 | 0.407824 | 0.413985 | 5.7341 | 1 | 3 | rs838069 | | |
| 0.0199778 | 1.1096 | 1558 | 0.332002 | 4626 | 0.309345 | 0.315063 | 5.4138 | 0.982361 | 4 | rs6542504 | 2 | rs12711923 |
| 0.02322 | 0.9 | 1558 | 0.716624 | 4627 | 0.737523 | 0.732257 | 5.152 | 0.999896 | 2 | rs4849760 | 3 | rs745888 |
| 0.0234152 | 0.9054 | 1558 | 0.339147 | 4627 | 0.361769 | 0.356092 | 5.1374 | 0.982618 | 2 | rs895398 | 4 | rs838066 |
| 0.0236887 | 1.1011 | 1555 | 0.397106 | 4610 | 0.374295 | 0.380049 | 5.1173 | 1 | 4 | rs6542504 | | |
| 0.0346288 | 1.0972 | 1522 | 0.658673 | 4559 | 0.63753 | 0.642822 | 4.4634 | 1 | 2 | rs838066 | | |
| 0.0376797 | 0.9131 | 1558 | 0.444559 | 4627 | 0.467115 | 0.461494 | 4.3194 | 0.903903 | 1 | rs838086 | 1 | rs838069 |
| 0.0612227 | 0.9009 | 1558 | 0.161601 | 4627 | 0.176246 | 0.172556 | 3.5039 | 0.98925 | 3 | rs6759589 | 2 | rs838066 |
| 0.0675678 | 1.0805 | 1558 | 0.403402 | 4627 | 0.384915 | 0.389572 | 3.3411 | 1 | 2 | rs11680207 | | |
| 0.0742724 | 1.1028 | 1558 | 0.20821 | 4627 | 0.192542 | 0.196469 | 3.186 | 0.887553 | 3 | rs1867750 | 2 | rs11680207 |
| 0.1275005 | 1.0674 | 1511 | 0.527134 | 3962 | 0.510853 | 0.515348 | 2.3227 | 1 | 4 | rs2084154 | | |
| 0.1817962 | 0.9326 | 1558 | 0.209889 | 4627 | 0.221696 | 0.218727 | 1.7829 | 0.931297 | 1 | rs10496555 | 3 | rs745888 |
| 0.2193745 | 1.056 | 1542 | 0.679313 | 4602 | 0.667319 | 0.670329 | 1.5085 | 1 | 2 | rs4849765 | | |
| 0.2349078 | 1.0589 | 1548 | 0.251292 | 4614 | 0.240681 | 0.243346 | 1.4109 | 1 | 2 | rs838086 | | |
| 0.2688289 | 0.9532 | 1558 | 0.456896 | 4627 | 0.468807 | 0.465537 | 1.2227 | 0.919405 | 3 | rs7562894 | 3 | rs2084154 |
| 0.2941036 | 0.9538 | 1558 | 0.331602 | 4627 | 0.342173 | 0.339281 | 1.1007 | 0.945087 | 4 | rs2084154 | 2 | rs895398 |
| 0.455931 | 1.0331 | 1558 | 0.354244 | 4627 | 0.346821 | 0.34869 | 0.5559 | 0.98416 | 2 | rs330763 | 3 | rs725346 |
| 0.4591499 | 1.033 | 1558 | 0.358115 | 4627 | 0.350681 | 0.352554 | 0.548 | 0.971987 | 3 | rs1439945 | 3 | rs725346 |
| 0.4793247 | 1.0562 | 1557 | 0.079641 | 4622 | 0.075725 | 0.076711 | 0.5004 | 1 | 1 | rs12711923 | | |
| 0.497656 | 1.03 | 1558 | 0.356544 | 4627 | 0.349799 | 0.351497 | 0.4599 | 0.989528 | 2 | rs330763 | 3 | rs10496556 |
| 0.5015258 | 0.9669 | 1558 | 0.216303 | 4627 | 0.222066 | 0.220614 | 0.4517 | 1 | 2 | rs11680207 | 2 | rs935361 |
| 0.5315142 | 1.0273 | 1553 | 0.370573 | 4610 | 0.364317 | 0.365893 | 0.3915 | 1 | 3 | rs725346 | | |
| 0.5977098 | 1.0229 | 1557 | 0.376365 | 4619 | 0.371076 | 0.372409 | 0.2785 | 1 | 3 | rs10496556 | | |
| 0.6233236 | 1.0256 | 1558 | 0.799101 | 4627 | 0.795007 | 0.796039 | 0.2412 | 1 | 2 | rs7564432 | | |
| 0.7140552 | 1.0158 | 1558 | 0.33668 | 4627 | 0.333192 | 0.334071 | 0.1343 | 1.054416 | 3 | rs10496555 | 3 | rs725346 |
| 0.7338904 | 0.9756 | 1558 | 0.084777 | 4627 | 0.086711 | 0.086224 | 0.1156 | 1.040852 | 3 | rs12711924 | 3 | rs838069 |
| 0.7373932 | 1.0483 | 1554 | 0.977479 | 4049 | 0.976416 | 0.976709 | 0.1124 | 1 | 2 | rs838087 | | |
| 0.7425392 | 1.0407 | 1558 | 0.970475 | 4627 | 0.96931 | 0.969604 | 0.1079 | 1 | 2 | rs4849760 | | |
| 0.8144482 | 1.01 | 1558 | 0.341581 | 4627 | 0.339335 | 0.339901 | 0.0551 | 1.052093 | 3 | rs10496555 | 3 | rs10496556 |
| 0.8352386 | 0.9855 | 1558 | 0.092358 | 4627 | 0.093587 | 0.093277 | 0.0433 | 1.03608 | 4 | rs330773 | 3 | rs745888 |
| 0.8666485 | 1.0069 | 1558 | 0.421023 | 4627 | 0.419347 | 0.419769 | 0.0282 | 1.049887 | 3 | rs10496555 | 1 | rs725346 |
| 0.8876881 | 1.0095 | 1558 | 0.106083 | 4627 | 0.10519 | 0.105415 | 0.0199 | 1.01195 | 1 | rs1867750 | 2 | rs7564432 |
| 0.915614 | 1.0149 | 1557 | 0.977521 | 4626 | 0.977194 | 0.977276 | 0.0112 | 1 | 3 | rs1545360 | | |
| 0.9646863 | 0.998 | 1558 | 0.700899 | 4627 | 0.701318 | 0.701213 | 0.002 | 1 | 3 | rs1867750 | | |
| 0.9767802 | 0.9959 | 1558 | 0.021916 | 4627 | 0.022003 | 0.021981 | 0.0008 | 1.023095 | 1 | rs1545360 | 1 | rs10165398 |

TABLE 8
Association of SNP markers and 2-marker haplotypes in the rs4848543 LD
block with risk for breast cancer (phenotype Medium Predisposition Breast Cancer):
The allele codes are as follows: 1 = A, 2 = C, 3 = G, 4 = T.

| p-value | RR | #aff | aff. freq | #con | con. freq | H0. freq | X2 | info_alt | all | mrk | all | mrk |
|---|---|---|---|---|---|---|---|---|---|---|---|---|
| 9.40E−10 | 1.4567 | 632 | 0.427215 | 4626 | 0.33863 | 0.349277 | 37.4465 | 1 | 1 | rs4848543 | | |
| 1.33E−06 | 0.748 | 632 | 0.464983 | 4627 | 0.537453 | 0.528745 | 23.3794 | 0.999257 | 2 | rs895398 | 3 | rs6759589 |
| 1.51E−06 | 1.3348 | 632 | 0.534019 | 4626 | 0.461954 | 0.470616 | 23.1368 | 1 | 1 | rs6759589 | | |
| 1.62E−06 | 1.3411 | 632 | 0.428798 | 4627 | 0.358872 | 0.367275 | 22.9946 | 1 | 1 | rs12711924 | | |
| 2.57E−06 | 1.4384 | 632 | 0.214003 | 4627 | 0.159157 | 0.165836 | 22.1127 | 0.970516 | 2 | rs4849765 | 4 | rs895398 |
| 4.01E−06 | 1.4104 | 632 | 0.224684 | 4626 | 0.17045 | 0.176968 | 21.2621 | 1 | 4 | rs895398 | | |
| 1.00E−05 | 1.3099 | 632 | 0.61788 | 4622 | 0.552466 | 0.560335 | 19.5116 | 1 | 3 | rs838100 | | |
| 0.0000125 | 1.3136 | 632 | 0.386076 | 4627 | 0.323752 | 0.331242 | 19.0859 | 1 | 4 | rs3731603 | | |
| 1.398E−05 | 1.3039 | 632 | 0.617089 | 4625 | 0.552757 | 0.560491 | 18.8713 | 1 | 3 | rs838102 | | |
| 0.0000142 | 0.7657 | 632 | 0.37216 | 4627 | 0.43636 | 0.428644 | 18.8419 | 0.995144 | 3 | rs6759589 | 1 | rs838102 |
| 1.579E−05 | 1.388 | 632 | 0.230115 | 4619 | 0.177183 | 0.184123 | 18.6401 | 0.943665 | 2 | rs4849765 | 4 | rs2084154 |
| 0.0000258 | 1.2911 | 632 | 0.588229 | 4627 | 0.525264 | 0.532825 | 17.7045 | 0.993478 | 2 | rs4849760 | 3 | rs838102 |
| 0.0004697 | 1.2366 | 631 | 0.444533 | 4626 | 0.392888 | 0.399087 | 12.2324 | 1 | 4 | rs1530561 | | |
| 0.0007986 | 0.7553 | 632 | 0.139112 | 4627 | 0.176242 | 0.171752 | 11.2444 | 0.995218 | 3 | rs6759589 | 2 | rs838066 |
| 0.0025521 | 1.2002 | 632 | 0.452532 | 4627 | 0.407824 | 0.413196 | 9.1029 | 1 | 3 | rs838069 | | |
| 0.0027736 | 1.2608 | 632 | 0.196994 | 4626 | 0.162884 | 0.166984 | 8.9507 | 1 | 4 | rs935361 | | |
| 0.0038501 | 1.5279 | 632 | 0.074928 | 4627 | 0.050342 | 0.053363 | 8.3532 | 0.690307 | 2 | rs838086 | 1 | rs12711924 |
| 0.0086964 | 0.8452 | 631 | 0.317499 | 4621 | 0.355011 | 0.350481 | 6.8841 | 0.989658 | 3 | rs2084154 | 4 | rs838066 |
| 0.0109182 | 1.1925 | 632 | 0.264011 | 4627 | 0.231241 | 0.235192 | 6.4786 | 0.998578 | 1 | rs745888 | 2 | rs838087 |
| 0.0116941 | 1.1903 | 632 | 0.264241 | 4625 | 0.231784 | 0.235686 | 6.3567 | 1 | 1 | rs745888 | | |
| 0.0125611 | 0.8469 | 632 | 0.704114 | 4627 | 0.737523 | 0.733507 | 6.2299 | 0.99995 | 2 | rs4849760 | 3 | rs745888 |
| 0.0140093 | 0.8557 | 632 | 0.326665 | 4627 | 0.361823 | 0.357582 | 6.037 | 0.997006 | 3 | rs6759589 | 4 | rs838066 |
| 0.0167713 | 1.1975 | 632 | 0.20807 | 4627 | 0.179922 | 0.183305 | 5.7202 | 1 | 2 | rs7564432 | 2 | rs11680207 |
| 0.0206458 | 0.8629 | 632 | 0.328408 | 4627 | 0.36172 | 0.357717 | 5.3565 | 0.986182 | 2 | rs895398 | 4 | rs838066 |
| 0.0250242 | 0.8675 | 632 | 0.431088 | 4627 | 0.466247 | 0.462051 | 5.0222 | 0.905031 | 1 | rs838086 | 1 | rs838069 |
| 0.0262532 | 0.772 | 632 | 0.068325 | 4627 | 0.086756 | 0.084542 | 4.9393 | 0.958473 | 3 | rs12711924 | 3 | rs838069 |
| 0.0331553 | 1.1454 | 621 | 0.668277 | 4559 | 0.63753 | 0.641216 | 4.5377 | 1 | 2 | rs838066 | | |
| 0.0420433 | 0.8752 | 632 | 0.312809 | 4627 | 0.342145 | 0.33827 | 4.1335 | 0.956363 | 4 | rs2084154 | 2 | rs895398 |
| 0.0490954 | 1.1279 | 632 | 0.413766 | 4627 | 0.384915 | 0.388382 | 3.8721 | 1 | 2 | rs11680207 | | |
| 0.0545878 | 1.1328 | 625 | 0.6944 | 4602 | 0.667319 | 0.670557 | 3.6946 | 1 | 2 | rs4849765 | | |
| 0.0565963 | 1.1614 | 632 | 0.216878 | 4627 | 0.19254 | 0.195451 | 3.6344 | 0.887609 | 3 | rs1867750 | 2 | rs11680207 |
| 0.1002333 | 1.1066 | 609 | 0.536125 | 3962 | 0.510853 | 0.51422 | 21018 | 1 | 4 | rs2084154 | | |
| 0.1098139 | 1.4252 | 630 | 0.983333 | 4049 | 0.976414 | 0.977346 | 2.5569 | 1 | 2 | rs838087 | | |
| 0.2444521 | 0.9299 | 632 | 0.450621 | 4627 | 0.46866 | 0.466267 | 1.3547 | 0.930417 | 3 | rs7562894 | 3 | rs2084154 |
| 0.2972591 | 1.0681 | 632 | 0.36608 | 4627 | 0.350928 | 0.352761 | 1.0865 | 0.9763 | 3 | rs1439945 | 3 | rs725346 |
| 0.324048 | 0.9409 | 632 | 0.600571 | 4626 | 0.615101 | 0.613351 | 0.9725 | 0.985757 | 2 | rs6542504 | 2 | rs12711923 |
| 0.3389162 | 1.0619 | 632 | 0.363391 | 4627 | 0.349623 | 0.351284 | 0.9145 | 0.992987 | 2 | rs330763 | 3 | rs10496556 |
| 0.3390482 | 0.9427 | 632 | 0.596933 | 4627 | 0.611053 | 0.609353 | 0.914 | 0.984363 | 2 | rs6542504 | 2 | rs4849760 |
| 0.3489783 | 1.0609 | 632 | 0.360144 | 4627 | 0.346646 | 0.348274 | 0.8772 | 0.98708 | 2 | rs330763 | 3 | rs725346 |
| 0.363347 | 1.0608 | 632 | 0.321891 | 4626 | 0.309141 | 0.310678 | 0.8263 | 0.984258 | 4 | rs6542504 | 2 | rs12711923 |
| 0.4496627 | 0.9444 | 632 | 0.212841 | 4627 | 0.222576 | 0.221424 | 0.5715 | 0.927154 | 1 | rs10496555 | 3 | rs745888 |
| 0.457771 | 1.047 | 631 | 0.385103 | 4610 | 0.374295 | 0.375596 | 0.5513 | 1 | 4 | rs6542504 | | |
| 0.4606554 | 0.9296 | 632 | 0.098529 | 4627 | 0.105201 | 0.104399 | 0.5443 | 1.01297 | 1 | rs1867750 | 2 | rs7564432 |
| 0.47764 | 1.0451 | 630 | 0.374603 | 4610 | 0.364317 | 0.365553 | 0.5042 | 1 | 3 | rs725346 | | |
| 0.4879223 | 1.1566 | 632 | 0.980221 | 4626 | 0.977194 | 0.977558 | 0.4811 | 1 | 3 | rs1545360 | | |
| 0.498098 | 1.045 | 632 | 0.343589 | 4627 | 0.333725 | 0.334913 | 0.459 | 0.948386 | 3 | rs10496555 | 3 | rs725346 |
| 0.5144968 | 1.0412 | 632 | 0.380538 | 4619 | 0.371076 | 0.372215 | 0.4249 | 1 | 3 | rs10496556 | | |
| 0.5466543 | 1.0398 | 632 | 0.348742 | 4627 | 0.339941 | 0.341001 | 0.3633 | 0.950967 | 3 | rs10496555 | 3 | rs10496556 |
| 0.5484699 | 0.8812 | 632 | 0.019407 | 4627 | 0.021965 | 0.021657 | 0.3601 | 1.017257 | 1 | rs1545360 | 1 | rs10165398 |
| 0.6700167 | 0.9696 | 632 | 0.216772 | 4627 | 0.222066 | 0.22143 | 0.1816 | 1 | 2 | rs11680207 | 2 | rs935361 |
| 0.7411272 | 0.9806 | 632 | 0.413979 | 4627 | 0.41875 | 0.418177 | 0.1091 | 1.048229 | 3 | rs10496555 | 1 | rs725346 |
| 0.7734425 | 0.971 | 632 | 0.090995 | 4627 | 0.093457 | 0.093161 | 0.0829 | 1.032733 | 4 | rs330773 | 3 | rs745888 |
| 0.8205628 | 1.0258 | 632 | 0.07753 | 4622 | 0.075725 | 0.075942 | 0.0514 | 1 | 1 | rs12711923 | | |
| 0.8541421 | 0.9688 | 632 | 0.968355 | 4627 | 0.969313 | 0.969196 | 0.0338 | 1 | 2 | rs4849760 | | |
| 0.9360615 | 0.9944 | 628 | 0.23965 | 4614 | 0.240681 | 0.240557 | 0.0064 | 1 | 2 | rs838086 | | |
| 0.9536622 | 0.9957 | 632 | 0.794304 | 4627 | 0.795007 | 0.794923 | 0.0034 | 1 | 2 | rs7564432 | | |
| 0.9754573 | 1.002 | 632 | 0.70174 | 4627 | 0.701318 | 0.701369 | 0.0009 | 1 | 3 | rs1867750 | | |

TABLE 9
Haplotype Diversity of the rs4848543 Right-half Sub-block:
(The allele codes are as follows: 1 = A, 2 = C, 3 = G, 4 = T)

| p-val | r | #aff | aff. freq | #con | con. freq | info_alt | |
|---|---|---|---|---|---|---|---|
| 2.65E−02 | 1.23 | 653 | 0.131 | 4480 | 0.109 | 0.96 | 2 rs895398 1 rs4848543 1 rs6759589 2 rs838066 3 rs838100 1 rs838086 1 rs12711924 |
| | | | | | | | 4 rs3731603 |
| 1.51E−02 | 1.26 | 653 | 0.123 | 4480 | 0.100 | 0.94 | 4 rs895398 1 rs4848543 1 rs6759589 2 rs838066 3 rs838100 1 rs838086 1 rs12711924 |
| | | | | | | | 4 rs3731603 |
| 1.70E−02 | 1.43 | 653 | 0.051 | 4480 | 0.036 | 0.90 | 4 rs895398 1 rs4848543 1 rs6759589 2 rs838066 3 rs838100 2 rs838086 1 rs12711924 |
| | | | | | | | 4 rs3731603 |
| 2.97E−01 | 1.17 | 653 | 0.043 | 4480 | 0.037 | 0.99 | 2 rs895398 1 rs4848543 1 rs6759589 2 rs838066 3 rs838100 1 rs838086 1 rs12711924 |
| | | | | | | | 3 rs3731603 |
| 1.19E−02 | 1.55 | 653 | 0.039 | 4480 | 0.026 | 0.89 | 4 rs895398 1 rs4848543 1 rs6759589 2 rs838066 3 rs838100 2 rs838086 3 rs12711924 |
| | | | | | | | 3 rs3731603 |
| 3.10E−01 | 1.36 | 653 | 0.015 | 4480 | 0.011 | 0.76 | 2 rs895398 1 rs4848543 1 rs6759589 2 rs838066 3 rs838100 2 rs838086 1 rs12711924 |
| | | | | | | | 4 rs3731603 |
| 2.08E−02 | 0.85 | 653 | 0.244 | 4480 | 0.274 | 0.97 | 2 rs895398 2 rs4848543 3 rs6759589 4 rs838066 1 rs838100 1 rs838086 3 rs12711924 |
| | | | | | | | 3 rs3731603 |
| 6.56E−03 | 0.77 | 653 | 0.107 | 4480 | 0.134 | 0.96 | 2 rs895398 2 rs4848543 3 rs6759589 2 rs838066 1 rs838100 1 rs838086 3 rs12711924 |
| | | | | | | | 3 rs3731603 |
| 5.95E−02 | 0.81 | 653 | 0.072 | 4480 | 0.088 | 0.96 | 2 rs895398 2 rs4848543 1 rs6759589 2 rs838066 3 rs838100 2 rs838086 3 rs12711924 |
| | | | | | | | 3 rs3731603 |
| 3.91E−01 | 0.88 | 653 | 0.043 | 4480 | 0.049 | 0.90 | 2 rs895398 2 rs4848543 3 rs6759589 4 rs838066 3 rs838100 2 rs838086 3 rs12711924 |
| | | | | | | | 3 rs3731603 |
| 3.00E−02 | 0.59 | 653 | 0.014 | 4480 | 0.024 | 0.83 | 2 rs895398 2 rs4848543 3 rs6759589 2 rs838066 3 rs838100 2 rs838086 3 rs12711924 |
| | | | | | | | 3 rs3731603 |
| 8.84E−01 | 1.03 | 653 | 0.022 | 4480 | 0.021 | 0.91 | 2 rs895398 2 rs4848543 1 rs6759589 2 rs838066 3 rs838100 1 rs838086 1 rs12711924 |
| | | | | | | | 4 rs3731603 |
| 8.33E−01 | 1.05 | 653 | 0.022 | 4480 | 0.021 | 0.91 | 2 rs895398 2 rs4848543 3 rs6759589 4 rs838066 3 rs838100 1 rs838086 1 rs12711924 |
| | | | | | | | 4 rs3731603 |
| 7.42E−01 | 1.09 | 653 | 0.015 | 4480 | 0.013 | 0.88 | 2 rs895398 2 rs4848543 1 rs6759589 2 rs838066 3 rs838100 1 rs838086 3 rs12711924 |
| | | | | | | | 3 rs3731603 |
| 6.26E−01 | 0.86 | 653 | 0.010 | 4480 | 0.012 | 0.91 | 2 rs895398 2 rs4848543 3 rs6759589 2 rs838066 1 rs838100 1 rs838086 1 rs12711924 |
| | | | | | | | 4 rs3731603 |

TABLE 10
HapMap SNP markers that are correlated with rs4848543 with r² value greater than 0.2:

| Surrogate | | | | | Pos in | Pos in | Pos in |
|---|---|---|---|---|---|---|---|
| SNP | Ref SNP | D′ | R² | p-value | Build 34 | Build 36 | SEQ ID 4 |
| rs11680207 | rs4848543 | 0.653174 | 0.309094 | 4.55E−09 | 120023583 | 119644908 | 301 |
| rs7583824 | rs4848543 | 0.655931 | 0.322684 | 3.29E−09 | 120027724 | 119649049 | 4442 |
| rs4848541 | rs4848543 | 0.656671 | 0.243502 | 8.45E−06 | 120032169 | 119653494 | 8887 |
| rs1446128 | rs4848543 | 0.628705 | 0.317935 | 8.08E−09 | 120032378 | 119653703 | 9096 |
| rs4849766 | rs4848543 | 0.851071 | 0.31689 | 7.16E−09 | 120034354 | 119655679 | 11072 |
| rs6715243 | rs4848543 | 0.923886 | 0.362632 | 3.83E−10 | 120034730 | 119656055 | 11448 |
| rs4142753 | rs4848543 | 0.934092 | 0.413776 | 2.96E−12 | 120051961 | 119673286 | 28679 |
| rs10188745 | rs4848543 | 0.934092 | 0.413776 | 2.96E−12 | 120057058 | 119678383 | 33776 |
| rs895398 | rs4848543 | 0.934092 | 0.413776 | 2.96E−12 | 120058180 | 119679505 | 34898 |
| rs4848543 | rs4848543 | 1 | 1 | 0 | 120061096 | 119682421 | 37814 |
| rs11684731 | rs4848543 | 1 | 0.319149 | 2.08E−12 | 120062319 | 119683644 | 39037 |
| rs12464139 | rs4848543 | 1 | 0.294118 | 1.69E−11 | 120062996 | 119684321 | 39714 |
| rs6754664 | rs4848543 | 1 | 0.963415 | 4.90E−33 | 120063070 | 119684395 | 39788 |
| rs6754799 | rs4848543 | 1 | 0.673648 | 6.09E−22 | 120063176 | 119684501 | 39894 |
| rs6759589 | rs4848543 | 1 | 0.534483 | 2.36E−19 | 120064976 | 119686301 | 41694 |
| rs1562256 | rs4848543 | 0.951061 | 0.50597 | 1.03E−15 | 120066318 | 119687643 | 43036 |
| rs1446125 | rs4848543 | 1 | 1 | 4.29E−31 | 120066620 | 119687945 | 43338 |
| rs1446124 | rs4848543 | 1 | 0.963415 | 4.90E−33 | 120066705 | 119688030 | 43423 |
| rs11677262 | rs4848543 | 1 | 0.30137 | 6.60E−12 | 120067290 | 119688615 | 44008 |
| rs7565771 | rs4848543 | 1 | 0.307692 | 2.81E−12 | 120070400 | 119691725 | 47118 |
| rs7595954 | rs4848543 | 1 | 0.289474 | 5.19E−12 | 120070666 | 119691991 | 47384 |
| rs838066 | rs4848543 | 1 | 0.3 | 2.40E−12 | 120071508 | 119692833 | 48226 |
| rs895397 | rs4848543 | 1 | 1 | 4.32E−35 | 120072400 | 119693725 | 49118 |
| rs838059 | rs4848543 | 0.841262 | 0.353861 | 2.97E−10 | 120075395 | 119696720 | 52113 |
| rs838102 | rs4848543 | 1 | 0.321918 | 4.92E−13 | 120079878 | 119701203 | 56596 |
| rs3769659 | rs4848543 | 0.849119 | 0.718225 | 2.57E−20 | 120081128 | 119702453 | 57846 |
| rs865108 | rs4848543 | 1 | 0.376923 | 4.62E−14 | 120081529 | 119702854 | 58247 |
| rs708670 | rs4848543 | 1 | 0.302817 | 4.27E−12 | 120082382 | 119703707 | 59100 |
| rs708672 | rs4848543 | 1 | 0.632075 | 3.65E−22 | 120082499 | 119703824 | 59217 |
| rs708673 | rs4848543 | 1 | 0.3125 | 1.13E−12 | 120082871 | 119704196 | 59589 |
| rs838100 | rs4848543 | 1 | 0.3 | 2.40E−12 | 120083710 | 119705035 | 60428 |
| rs838098 | rs4848543 | 1 | 0.3125 | 1.13E−12 | 120084226 | 119705551 | 60944 |
| rs838096 | rs4848543 | 1 | 0.310811 | 1.10E−12 | 120084741 | 119706066 | 61459 |
| rs838092 | rs4848543 | 1 | 0.321918 | 4.92E−13 | 120091298 | 119712623 | 68016 |
| rs838090 | rs4848543 | 0.780443 | 0.224773 | 5.57E−06 | 120092304 | 119713629 | 69022 |
| rs12467944 | rs4848543 | 0.865803 | 0.535809 | 9.44E−15 | 120094306 | 119715631 | 71024 |
| rs10196144 | rs4848543 | 0.84638 | 0.686857 | 4.46E−19 | 120095312 | 119716637 | 72030 |
| rs6728660 | rs4848543 | 0.872481 | 0.70845 | 3.68E−18 | 120095756 | 119717081 | 72474 |
| rs10188946 | rs4848543 | 0.74499 | 0.446479 | 2.07E−12 | 120098925 | 119720250 | 75643 |
| rs895406 | rs4848543 | 0.790753 | 0.304687 | 1.52E−09 | 120104365 | 119725690 | 81083 |
| rs708675 | rs4848543 | 0.707874 | 0.418005 | 1.81E−11 | 120104502 | 119725827 | 81220 |
| rs1867856 | rs4848543 | 0.77707 | 0.512514 | 2.87E−13 | 120104672 | 119725997 | 81390 |
| rs3754847 | rs4848543 | 0.717855 | 0.22469 | 4.70E−07 | 120105720 | 119727045 | 82438 |
| rs12711924 | rs4848543 | 0.792931 | 0.561965 | 3.43E−15 | 120107316 | 119728641 | 84034 |
| rs1867749 | rs4848543 | 0.75345 | 0.214069 | 6.37E−07 | 120109057 | 119730382 | 85775 |
| rs838073 | rs4848543 | 0.709981 | 0.247536 | 5.75E−07 | 120109754 | 119731079 | 86472 |
| rs3731603 | rs4848543 | 0.784597 | 0.392356 | 2.49E−10 | 120117062 | 119738387 | 93780 |

TABLE 11
Association of SNP SG02S728 (rs13387042) allele A with risk for breast cancer:

| Cohort | p-val. corr* | p-val | r | 95% CI | #aff | aff. freq | #con | con. freq |
|---|---|---|---|---|---|---|---|---|
| MedPre BC Illumina | 4.4E−03 | 2.1E−03 | 1.20 | | 652 | 0.496 | 4475 | 0.451 |
| MedPre BC Iceland Replication | 2.3E−03 | 1.8E−03 | 1.37 | | 200 | 0.528 | 7966 | 0.449 |
| MedPre BC All Icelandic | 8.5E−05 | 1.5E−05 | 1.24 | (1.12, 1.38) | 852 | 0.504 | 12441 | 0.449 |
| Any BC Illumina | 4.0E−05 | 2.1E−05 | 1.19 | | 1598 | 0.494 | 4475 | 0.451 |
| Any BC Icelandic Replication | 3.8E−03 | 3.3E−03 | 1.20 | | 583 | 0.493 | 7966 | 0.449 |
| Any BC All Icelandic | 2.0E−07 | 5.0E−08 | 1.20 | (1.12, 1.28) | 2181 | 0.494 | 12441 | 0.449 |
| Spain | | 1.8E−02 | 1.21 | (1.03, 1.42) | 446 | 0.581 | 977 | 0.533 |
| Sweden Familial | | 2.2E−01 | 1.11 | | 346 | 0.516 | 1734 | 0.490 |
| Sweden Consecutive | | 2.0E−04 | 1.31 | | 482 | 0.558 | 1734 | 0.490 |
| Sweden All | | 8.1E−04 | 1.22 | (1.09, 1.37) | 828 | 0.540 | 1734 | 0.490 |
| All cohorts joint | 3.8E−11 | | 1.20 | (1.14, 1.27) | 3455 | 0.516 | 15152 | 0.460 |

*corrected for relationships between individuals

TABLE 12
Definition of the rs13387042 LD-block on Chromosome 2:

| NCBI Build | Start | End |
|---|---|---|
| 34 | 218,062,001 | 218,141,002 |
| 36 | 217,567,902 | 217,646,679 |

TABLE 13
Haplotype Diversity of the rs13387042 LD-block:
The allele codes are as follows: 1 = A, 2 = C, 3 = G, 4 = T.

| p-val | r | #aff | aff. frq. | #con | con. frq. | info | 84 | 7 | 45 | 2 | 9 | 30 | 4 | 9 | 3 | 42 | 44 | 60 |
|---|---|---|---|---|---|---|---|---|---|---|---|---|---|---|---|---|---|---|
| 8.4E−03 | 1.12 | 1599 | 0.283 | 11122 | 0.261 | 0.99 | 3 | 4 | 3 | 1 | 4 | 1 | 1 | 2 | 3 | 1 | 1 | 3 |
| 2.7E−03 | 1.18 | 1599 | 0.144 | 11122 | 0.125 | 0.98 | 3 | 4 | 3 | 3 | 4 | 1 | 2 | 4 | 3 | 1 | 3 | 3 |
| 3.0E−01 | 1.13 | 1599 | 0.031 | 11122 | 0.027 | 0.96 | 3 | 2 | 3 | 3 | 4 | 1 | 2 | 4 | 3 | 1 | 3 | 3 |
| 2.9E−01 | 0.85 | 1599 | 0.014 | 11122 | 0.017 | 1.00 | 3 | 4 | 3 | 3 | 4 | 1 | 1 | 2 | 3 | 1 | 3 | 3 |
| 6.4E−02 | 0.65 | 1599 | 0.006 | 11122 | 0.009 | 1.00 | 3 | 4 | 1 | 3 | 2 | 1 | 1 | 2 | 3 | 1 | 1 | 3 |
| 3.0E−02 | 2.24 | 1599 | 0.005 | 11122 | 0.002 | 0.72 | 3 | 4 | 3 | 3 | 4 | 1 | 2 | 4 | 3 | 1 | 3 | 1 |
| 3.2E−02 | 2.79 | 1599 | 0.004 | 11122 | 0.001 | 0.57 | 3 | 4 | 3 | 1 | 4 | 1 | 1 | 2 | 3 | 1 | 1 | 1 |
| 7.6E−01 | 1.13 | 1599 | 0.003 | 11122 | 0.003 | 0.75 | 3 | 2 | 3 | 1 | 4 | 1 | 1 | 2 | 3 | 1 | 1 | 3 |
| 1.6E−01 | 1.70 | 1599 | 0.003 | 11122 | 0.002 | 1.00 | 3 | 4 | 3 | 1 | 4 | 1 | 1 | 2 | 3 | 1 | 3 | 1 |
| 6.7E−01 | 1.27 | 1599 | 0.001 | 11122 | 0.001 | 0.99 | 3 | 2 | 3 | 3 | 4 | 1 | 1 | 2 | 3 | 1 | 1 | 3 |
| 4.3E−02 | 0.91 | 1599 | 0.242 | 11122 | 0.258 | 0.99 | 3 | 2 | 3 | 3 | 2 | 3 | 1 | 2 | 3 | 3 | 1 | 3 |
| 8.1E−02 | 0.91 | 1599 | 0.128 | 11122 | 0.139 | 0.99 | 3 | 4 | 3 | 3 | 4 | 1 | 1 | 4 | 1 | 3 | 3 | 1 |
| 1.4E−01 | 0.88 | 1599 | 0.049 | 11122 | 0.055 | 0.98 | 1 | 4 | 3 | 3 | 4 | 1 | 1 | 4 | 1 | 3 | 3 | 1 |
| 1.1E−01 | 0.84 | 1599 | 0.031 | 11122 | 0.036 | 0.95 | 3 | 2 | 3 | 3 | 2 | 3 | 1 | 2 | 3 | 3 | 1 | 1 |
| 9.2E−01 | 0.99 | 1599 | 0.016 | 11122 | 0.016 | 1.00 | 1 | 2 | 1 | 3 | 2 | 1 | 1 | 2 | 3 | 3 | 3 | 3 |
| 6.3E−01 | 0.93 | 1599 | 0.015 | 11122 | 0.016 | 0.96 | 3 | 4 | 3 | 3 | 4 | 1 | 1 | 4 | 1 | 3 | 3 | 3 |
| 6.3E−01 | 1.09 | 1599 | 0.012 | 11122 | 0.011 | 0.92 | 1 | 2 | 3 | 3 | 4 | 1 | 1 | 4 | 1 | 3 | 3 | 1 |
| 4.0E−01 | 0.80 | 1599 | 0.005 | 11122 | 0.006 | 0.98 | 3 | 4 | 3 | 3 | 4 | 1 | 1 | 2 | 3 | 3 | 1 | 3 |
| 6.4E−01 | 0.87 | 1599 | 0.004 | 11122 | 0.004 | 1.00 | 3 | 2 | 3 | 3 | 2 | 1 | 1 | 2 | 3 | 3 | 1 | 3 |
| 1.9E−01 | 0.49 | 1599 | 0.001 | 11122 | 0.002 | 0.86 | 1 | 2 | 3 | 3 | 2 | 3 | 1 | 2 | 3 | 3 | 1 | 3 |
| 2.9E−01 | 0.49 | 1599 | 0.001 | 11122 | 0.001 | 0.99 | 3 | 4 | 3 | 3 | 4 | 1 | 1 | 4 | 3 | 3 | 3 | 1 |

TABLE 14
Association of SNP markers and 2-marker haplotypes in the rs13387042 LD-
block with risk for breast cancer (phenotype Any Breast Cancer):
The allele codes are as follows: 1 = A, 2 = C, 3 = G, 4 = T.

| pvcorr | r | #aff | aff. freq | #con | con. freq | allele | marker | allele | marker |
|---|---|---|---|---|---|---|---|---|---|
| 7.58E−06 | 1.1913 | 1658 | 0.493969 | 11547 | 0.450377 | 1 | rs13387042 | | |
| 8.59E−04 | 1.1892 | 1660 | 0.178916 | 11556 | 0.154855 | 2 | rs6735174 | | |
| 8.04E−03 | 1.1216 | 1660 | 0.29247 | 11563 | 0.269307 | 1 | rs6716542 | | |
| 1.17E−02 | 1.1281 | 1660 | 0.796385 | 11561 | 0.776144 | 3 | rs2372943 | | |
| 1.24E−02 | 1.1214 | 1660 | 0.769879 | 11560 | 0.748962 | 3 | rs13011060 | | |
| 1.54E−02 | 1.1074 | 1659 | 0.6956 | 11560 | 0.673573 | 4 | rs4491709 | | |
| 2.65E−02 | 1.1007 | 1659 | 0.723026 | 11561 | 0.703399 | 1 | rs12621130 | | |
| 1.03E−01 | 1.0691 | 1660 | 0.655723 | 11563 | 0.640491 | 4 | rs6435957 | | |
| 2.37E−01 | 1.088 | 1659 | 0.920735 | 11560 | 0.91436 | 3 | rs10191184 | | |
| 7.63E−01 | 1.0122 | 1660 | 0.383133 | 11560 | 0.380277 | 4 | rs6435959 | | |
| 7.74E−01 | 1.0367 | 1660 | 0.975602 | 11555 | 0.974729 | 3 | rs10171745 | | |
| 8.45E−01 | 1.0078 | 1660 | 0.417169 | 11563 | 0.41529 | 3 | rs10490444 | | |
| 9.54E−06 | 0.8405 | 1660 | 0.489993 | 11563 | 0.533373 | 3 | rs10171745 | 3 | rs13387042 |
| 4.24E−03 | 0.8771 | 1659 | 0.22895 | 11562 | 0.25292 | 1 | rs12621130 | 3 | rs13387042 |
| 6.79E−03 | 1.1253 | 1660 | 0.288931 | 11563 | 0.265289 | 4 | rs6435957 | 1 | rs6716542 |
| 7.96E−03 | 1.1219 | 1660 | 0.29198 | 11563 | 0.268789 | 1 | rs6716542 | 4 | rs4491709 |
| 8.04E−03 | 1.1216 | 1660 | 0.29247 | 11563 | 0.269307 | 1 | rs6716542 | 2 | rs6435959 |
| 8.04E−03 | 1.1216 | 1660 | 0.29247 | 11563 | 0.269307 | 3 | rs10171745 | 1 | rs6716542 |
| 9.38E−03 | 0.8938 | 1660 | 0.286833 | 11563 | 0.310333 | 2 | rs4491709 | 1 | rs10490444 |
| 1.03E−02 | 1.118 | 1660 | 0.289694 | 11563 | 0.267286 | 1 | rs6716542 | 1 | rs10490444 |
| 1.29E−02 | 0.9003 | 1660 | 0.683133 | 11563 | 0.705426 | 3 | rs10171745 | 3 | rs6716542 |
| 1.80E−02 | 0.8986 | 1660 | 0.304278 | 11563 | 0.327368 | 4 | rs2705593 | 3 | rs13387042 |
| 2.92E−02 | 1.0966 | 1660 | 0.315096 | 11562 | 0.295536 | 1 | rs6735174 | 1 | rs13387042 |
| 3.27E−02 | 1.0947 | 1660 | 0.312348 | 11563 | 0.293242 | 4 | rs4491709 | 2 | rs6435959 |
| 3.40E−02 | 0.9119 | 1660 | 0.275355 | 11563 | 0.294127 | 3 | rs10191184 | 3 | rs12621130 |
| 3.60E−02 | 0.9128 | 1660 | 0.274287 | 11563 | 0.29282 | 4 | rs2705593 | 3 | rs12621130 |
| 6.69E−02 | 1.0758 | 1660 | 0.405656 | 11563 | 0.388167 | 3 | rs10171745 | 1 | rs4674132 |
| 1.08E−01 | 0.9339 | 1660 | 0.315371 | 11563 | 0.33033 | 3 | rs10191184 | 2 | rs6435957 |
| 1.27E−01 | 1.0702 | 1660 | 0.293447 | 11563 | 0.279572 | 4 | rs6435957 | 1 | rs10490444 |
| 1.53E−01 | 1.0653 | 1660 | 0.270411 | 11563 | 0.258112 | 4 | rs2705593 | 4 | rs6435957 |
| 3.74E−01 | 1.0381 | 1660 | 0.335709 | 11563 | 0.327421 | 2 | rs2705593 | 3 | rs10191184 |
| 6.52E−01 | 0.9825 | 1660 | 0.544171 | 11563 | 0.548548 | 1 | rs12621130 | 1 | rs6735174 |
| 6.88E−01 | 0.984 | 1660 | 0.61747 | 11563 | 0.621271 | 1 | rs6735174 | 3 | rs2372943 |
| 7.59E−01 | 0.958 | 1660 | 0.028119 | 11563 | 0.029317 | 1 | rs10191184 | 2 | rs6435957 |
| 8.45E−01 | 0.9923 | 1660 | 0.582831 | 11563 | 0.58471 | 1 | rs6735174 | 1 | rs10490444 |
| 8.97E−01 | 0.9947 | 1660 | 0.355026 | 11563 | 0.356245 | 2 | rs2705593 | 3 | rs10490444 |
| 9.07E−01 | 0.9954 | 1660 | 0.403688 | 11563 | 0.404807 | 3 | rs6716542 | 4 | rs4491709 |
| 9.61E−01 | 1.0068 | 1660 | 0.026373 | 11563 | 0.026199 | 4 | rs2705593 | 1 | rs10191184 |

TABLE 15
HapMap SNP markers that are correlated with rs13387042 with an r² value
greater than 0.2:

| Surrogate | | | | | Pos in | Pos in | Pos in |
|---|---|---|---|---|---|---|---|
| SNP | Ref SNP | D′ | R2 | p-value | Build 34 | Build 36 | SEQ ID 5 |
| rs1882419 | rs13387402 | 0.692153 | 0.331807 | 1.92E−09 | 218059508 | 217565211 | 301 |
| rs2252488 | rs13387402 | 1 | 0.258085 | 5.60E−11 | 218059992 | 217565695 | 785 |
| rs2272525 | rs13387402 | 0.849123 | 0.271771 | 1.07E−08 | 218061250 | 217566904 | 1994 |
| rs4396687 | rs13387402 | 1 | 0.268293 | 1.29E−10 | 218063010 | 217568713 | 3803 |
| rs12614767 | rs13387402 | 0.785371 | 0.245063 | 1.01E−07 | 218063508 | 217569211 | 4301 |
| rs4571035 | rs13387402 | 1 | 0.344501 | 1.91E−13 | 218064351 | 217570054 | 5144 |
| rs10195963 | rs13387402 | 1 | 0.324895 | 7.05E−13 | 218065238 | 217570941 | 6031 |
| rs10207736 | rs13387402 | 0.573221 | 0.236068 | 4.26E−07 | 218066096 | 217571799 | 6889 |
| rs12464018 | rs13387402 | 0.847812 | 0.263762 | 1.33E−08 | 218067143 | 217572846 | 7936 |
| rs10932689 | rs13387402 | 0.880377 | 0.389169 | 5.54E−12 | 218068532 | 217574235 | 9325 |
| rs12613030 | rs13387402 | 0.917661 | 0.281049 | 3.18E−09 | 218068906 | 217574609 | 9699 |
| rs2888450 | rs13387402 | 1 | 0.323308 | 4.19E−13 | 218069383 | 217575086 | 10176 |
| rs4254482 | rs13387402 | 1 | 0.304413 | 5.14E−12 | 218069959 | 217575662 | 10752 |
| rs2372932 | rs13387402 | 1 | 0.268293 | 2.98E−11 | 218069975 | 217575678 | 10768 |
| rs4255939 | rs13387402 | 1 | 0.300466 | 2.28E−12 | 218070431 | 217576134 | 11224 |
| rs10192415 | rs13387402 | 1 | 0.33518 | 1.75E−13 | 218073196 | 217578899 | 13989 |
| rs7595393 | rs13387402 | 1 | 0.319631 | 7.19E−13 | 218073796 | 217579499 | 14589 |
| rs2372935 | rs13387402 | 1 | 0.331897 | 4.68E−13 | 218076131 | 217581834 | 16924 |
| rs6733648 | rs13387402 | 1 | 0.318182 | 6.69E−13 | 218077438 | 217583141 | 18231 |
| rs6435957 | rs13387402 | 0.586033 | 0.244261 | 1.46E−07 | 218080751 | 217586454 | 21544 |
| rs12614773 | rs13387402 | 0.911373 | 0.292062 | 1.74E−08 | 218084485 | 217590188 | 25278 |
| rs17833842 | rs13387402 | 1 | 0.345455 | 5.50E−12 | 218087663 | 217593366 | 28456 |
| rs10177578 | rs13387402 | 1 | 0.331897 | 2.95E−13 | 218088201 | 217593904 | 28994 |
| rs12623304 | rs13387402 | 0.919746 | 0.300741 | 1.15E−09 | 218090342 | 217596045 | 31135 |
| rs6716542 | rs13387402 | 1 | 0.33518 | 1.75E−13 | 218093185 | 217598888 | 33978 |
| rs17777330 | rs13387402 | 1 | 0.33518 | 1.75E−13 | 218094234 | 217599937 | 35027 |
| rs10211546 | rs13387402 | 1 | 0.312977 | 1.07E−12 | 218094939 | 217600642 | 35732 |
| rs2372937 | rs13387402 | 0.818494 | 0.335949 | 6.72E−10 | 218095256 | 217600959 | 36049 |
| rs4315498 | rs13387402 | 1 | 0.345455 | 5.50E−12 | 218095412 | 217601115 | 36205 |
| rs12998806 | rs13387402 | 1 | 0.33395 | 1.54E−11 | 218096305 | 217602008 | 37098 |
| rs12465515 | rs13387402 | 1 | 0.347368 | 7.21E−14 | 218096945 | 217602648 | 37738 |
| rs4491709 | rs13387402 | 0.81627 | 0.334787 | 8.36E−10 | 218097298 | 217603001 | 38091 |
| rs2372938 | rs13387402 | 0.818494 | 0.335949 | 6.72E−10 | 218097651 | 217603354 | 38444 |
| rs12621130 | rs13387402 | 1 | 0.345455 | 5.50E−12 | 218098168 | 217603871 | 38961 |
| rs13399995 | rs13387402 | 1 | 0.321168 | 7.70E−13 | 218100083 | 217605786 | 40876 |
| rs12052807 | rs13387402 | 0.788298 | 0.255063 | 4.96E−08 | 218100783 | 217606486 | 41576 |
| rs10199394 | rs13387402 | 1 | 0.347368 | 7.21E−14 | 218101273 | 217606976 | 42066 |
| rs2372940 | rs13387402 | 1 | 0.354179 | 2.00E−13 | 218101566 | 217607269 | 42359 |
| rs4583440 | rs13387402 | 0.818494 | 0.335949 | 6.72E−10 | 218101794 | 217607497 | 42587 |
| rs2372941 | rs13387402 | 1 | 0.315673 | 1.25E−12 | 218101850 | 217607553 | 42643 |
| rs6721811 | rs13387402 | 1 | 0.347368 | 7.21E−14 | 218102838 | 217608541 | 43631 |
| rs12615418 | rs13387402 | 1 | 0.345455 | 5.50E−12 | 218104345 | 217610048 | 45138 |
| rs12621884 | rs13387402 | 1 | 0.340033 | 2.71E−11 | 218104452 | 217610155 | 45245 |
| rs2372943 | rs13387402 | 1 | 0.345455 | 5.50E−12 | 218106318 | 217612021 | 47111 |
| rs4522583 | rs13387402 | 1 | 0.431818 | 1.56E−14 | 218106548 | 217612251 | 47341 |
| rs2888451 | rs13387402 | 1 | 0.33518 | 1.75E−13 | 218107055 | 217612758 | 47848 |
| rs2270398 | rs13387402 | 1 | 0.33395 | 1.84E−11 | 218107518 | 217613221 | 48311 |
| rs13412666 | rs13387402 | 1 | 1 | 5.65E−36 | 218108321 | 217614024 | 49114 |
| rs13387402 | rs13387402 | 1 | 1 | 0 | 218108374 | 217614077 | 49167 |
| rs13426489 | rs13387402 | 1 | 1 | 1.42E−35 | 218108788 | 217614491 | 49581 |
| rs6713249 | rs13387402 | 1 | 0.345455 | 5.50E−12 | 218109630 | 217615333 | 50423 |
| rs12620095 | rs13387402 | 1 | 0.863636 | 3.14E−29 | 218110056 | 217615759 | 50849 |
| rs4621152 | rs13387402 | 0.782931 | 0.262305 | 4.22E−09 | 218111527 | 217617230 | 52320 |
| rs6721996 | rs13387402 | 1 | 0.964912 | 4.45E−34 | 218112005 | 217617708 | 52798 |
| rs12622764 | rs13387402 | 1 | 0.345455 | 5.50E−12 | 218112403 | 217618106 | 53196 |
| rs6723013 | rs13387402 | 1 | 0.331897 | 4.68E−13 | 218113452 | 217619155 | 54245 |
| rs2372945 | rs13387402 | 1 | 0.385965 | 4.45E−15 | 218116654 | 217622357 | 57447 |
| rs12694403 | rs13387402 | 0.934804 | 0.388184 | 4.75E−12 | 218117956 | 217623659 | 58749 |
| rs12613955 | rs13387402 | 1 | 0.863636 | 4.73E−29 | 218118594 | 217624297 | 59387 |
| rs11894340 | rs13387402 | 0.781203 | 0.257717 | 6.21E−09 | 218119709 | 217625412 | 60502 |
| rs17778329 | rs13387402 | 1 | 0.35514 | 5.16E−12 | 218122353 | 217628056 | 63146 |
| rs4442975 | rs13387402 | 1 | 0.964912 | 4.45E−34 | 218123311 | 217629014 | 64104 |
| rs10932693 | rs13387402 | 1 | 0.324895 | 7.05E−13 | 218123663 | 217629366 | 64456 |
| rs17778427 | rs13387402 | 1 | 0.345455 | 5.50E−12 | 218125555 | 217631258 | 66348 |
| rs17835044 | rs13387402 | 1 | 0.345455 | 5.50E−12 | 218126147 | 217631850 | 66940 |
| rs7562029 | rs13387402 | 1 | 0.33518 | 1.75E−13 | 218126803 | 217632506 | 67596 |
| rs13000023 | rs13387402 | 0.923607 | 0.31255 | 5.05E−10 | 218126936 | 217632639 | 67729 |
| rs735361 | rs13387402 | 1 | 0.345455 | 5.50E−12 | 218127665 | 217633368 | 68458 |
| rs13409592 | rs13387402 | 1 | 0.210526 | 2.27E−09 | 218128870 | 217634573 | 69663 |
| rs2287289 | rs13387402 | 0.856459 | 0.284427 | 2.96E−09 | 218134039 | 217639742 | 74832 |
| rs12329133 | rs13387402 | 0.722969 | 0.239375 | 1.69E−07 | 218137658 | 217643361 | 78451 |
| rs13022815 | rs13387402 | 0.722969 | 0.239375 | 1.69E−07 | 218138666 | 217644369 | 79459 |
| rs13011060 | rs13387402 | 0.849849 | 0.279401 | 1.41E−08 | 218140719 | 217646422 | 81512 |
| rs13011326 | rs13387402 | 0.853195 | 0.289251 | 9.17E−09 | 218140952 | 217646655 | 81745 |
| rs4674132 | rs13387402 | 0.725082 | 0.249037 | 1.45E−07 | 218141061 | 217646764 | 81854 |

TABLE 16
Association of SNP SG16S299 (rs3803662) allele T with risk for breast cancer:
Association results for the allele T of the SNP rs3803662 on 16q12. Iceland 1
is the initial discovery cohort and Iceland 2 the independent replication group.
All P-values are two-sided and are adjusted for relatedness and other potential
population stratification of the Icelandic cases and controls. For combined
non-Icelanders and for all cohorts combined the OR and P-values are calculated
using the Mantel-Haenszel method and the displayed frequencies are obtained
as a simple (arithmetic) average over the frequencies of individual groups.

| rs3803662 (T) | Frequency | | | | | |
|---|---|---|---|---|---|---|
| Cohort (Cases/Controls) | Cases | Controls | OR | (95% CI) | P | PAR |
| Iceland 1 (1600/11563) | 0.278 | 0.238 | 1.23 | (1.13, 1.34) | 4.7E−06 | |
| Iceland 2 (594/1433) | 0.274 | 0.227 | 1.28 | (1.08, 1.52) | 4.9E−03 | |
| Iceland combined | 0.276 | 0.237 | 1.23 | (1.14, 1.33) | 2.8E−07 | 0.101 |
| (2194/12996) | | | | | | |
| Sweden (819/1723) | 0.333 | 0.250 | 1.50 | (1.32, 1.71) | 8.3E−10 | 0.209 |
| Spain (455/1002) | 0.348 | 0.308 | 1.20 | (1.02, 1.42) | 3.1E−02 | 0.114 |
| Holland (558/1384) | 0.306 | 0.255 | 1.29 | (1.10, 1.50) | 1.4E−03 | 0.131 |
| Non-Icelanders | 0.329 | 0.271 | 1.35 | (1.24, 1.47) | 5.1E−12 | 0.166 |
| (1832/4109) | | | | | | |
| All cohorts | 0.316 | 0.262 | 1.28 | (1.21, 1.36) | 2.7E−17 | 0.134 |
| (4026/17105) | | | | | | |

TABLE 17
Definition of the rs3803662 LD-block on Chromosome 16:

| NCBI Build | Start | End |
|---|---|---|
| 34 | 52,291,041 | 52,436,127 |
| 36 | 51,069,957 | 51,215,026 |

TABLE 18
Association of SNP markers and 2-marker haplotypes in the rs3803662 LD-block
with risk for breast cancer (phenotype Any Breast Cancer):
The allele codes are as follows: 1 = A, 2 = C, 3 = G, 4 = T.

| p-val | r | #aff | aff. frq. | #con | con. frq. | info | rs1111480 | rs187679 | rs9302555 |
|---|---|---|---|---|---|---|---|---|---|
| 2.46E−06 | 1.23 | 1715 | 0.283 | 12971 | 0.242 | 1.00 | | | |
| 4.35E−06 | 1.23 | 1715 | 0.267 | 12970 | 0.228 | 1.00 | | | |
| 5.16E−06 | 1.25 | 1715 | 0.234 | 12972 | 0.196 | 0.93 | | 4 | |
| 6.12E−06 | 1.23 | 1715 | 0.274 | 12972 | 0.235 | 0.99 | | | |
| 6.91E−06 | 1.22 | 1715 | 0.280 | 12971 | 0.241 | 0.99 | | | |
| 7.39E−06 | 1.23 | 1714 | 0.266 | 12967 | 0.228 | 0.98 | | | 4 |
| 8.18E−06 | 1.23 | 1715 | 0.266 | 12970 | 0.228 | 0.99 | | | |
| 8.36E−06 | 1.23 | 1715 | 0.256 | 12973 | 0.218 | 0.95 | | | |
| 9.13E−06 | 1.22 | 1713 | 0.273 | 12971 | 0.235 | 1.00 | | | |
| 9.86E−06 | 1.23 | 1713 | 0.244 | 12971 | 0.207 | 0.97 | | | |
| 1.06E−05 | 1.24 | 1715 | 0.240 | 12967 | 0.203 | 0.93 | 1 | | |
| 1.08E−05 | 0.82 | 1712 | 0.725 | 12953 | 0.763 | 1.00 | | | |
| 1.08E−05 | 1.22 | 1712 | 0.275 | 12953 | 0.237 | 1.00 | | | |
| 1.10E−05 | 1.22 | 1715 | 0.266 | 12972 | 0.229 | 0.98 | | | |
| 1.30E−05 | 1.22 | 1712 | 0.271 | 12965 | 0.234 | 1.00 | | | |
| 2.67E−05 | 1.22 | 1715 | 0.254 | 12973 | 0.218 | 0.94 | | | |
| 3.31E−05 | 1.21 | 1715 | 0.324 | 12971 | 0.284 | 0.87 | | 4 | |
| 6.62E−05 | 1.20 | 1715 | 0.322 | 12971 | 0.283 | 0.86 | | 4 | |
| 1.04E−04 | 1.18 | 1715 | 0.358 | 12970 | 0.321 | 0.98 | 1 | | |
| 1.05E−04 | 1.20 | 1715 | 0.292 | 12973 | 0.255 | 0.86 | | 4 | |
| 2.30E−04 | 1.17 | 1711 | 0.356 | 12952 | 0.321 | 0.98 | 1 | | |
| 5.78E−04 | 0.87 | 1715 | 0.370 | 12972 | 0.404 | 0.94 | | 2 | |
| 1.24E−03 | 1.15 | 1715 | 0.325 | 12973 | 0.295 | 0.94 | 1 | | |
| 1.39E−03 | 1.14 | 1714 | 0.458 | 12972 | 0.426 | 0.96 | | | 4 |
| 1.89E−03 | 1.14 | 1714 | 0.390 | 12971 | 0.360 | 0.95 | | | 4 |
| 1.99E−03 | 1.13 | 1714 | 0.457 | 12962 | 0.426 | 0.96 | | | 4 |
| 2.01E−03 | 1.13 | 1714 | 0.588 | 12958 | 0.558 | 0.99 | 1 | | 4 |
| 2.19E−03 | 1.13 | 1715 | 0.585 | 12971 | 0.555 | 1.00 | 1 | 4 | |
| 2.36E−03 | 0.89 | 1715 | 0.415 | 12960 | 0.445 | 1.00 | | 2 | |
| 2.36E−03 | 1.13 | 1715 | 0.585 | 12960 | 0.555 | 1.00 | | 4 | |
| 2.38E−03 | 1.13 | 1715 | 0.585 | 12970 | 0.555 | 1.00 | | 4 | 4 |
| 2.67E−03 | 1.13 | 1715 | 0.545 | 12972 | 0.515 | 0.96 | | | 4 |
| 3.37E−03 | 0.87 | 1715 | 0.272 | 12973 | 0.301 | 0.86 | | 2 | |
| 3.43E−03 | 0.86 | 1715 | 0.238 | 12971 | 0.266 | 0.84 | | 2 | |
| 3.44E−03 | 1.13 | 1715 | 0.496 | 12973 | 0.467 | 0.93 | | 4 | |
| 3.45E−03 | 0.86 | 1715 | 0.239 | 12971 | 0.266 | 0.84 | | 2 | |
| 3.64E−03 | 0.89 | 1713 | 0.541 | 12971 | 0.570 | 0.97 | | | |
| 4.13E−03 | 1.12 | 1712 | 0.492 | 12956 | 0.463 | 1.00 | | | |
| 4.13E−03 | 0.89 | 1712 | 0.508 | 12956 | 0.537 | 1.00 | | | |
| 4.51E−03 | 0.89 | 1713 | 0.508 | 12970 | 0.536 | 1.00 | | | |
| 4.58E−03 | 0.89 | 1713 | 0.506 | 12971 | 0.534 | 1.00 | | | |
| 4.71E−03 | 0.89 | 1713 | 0.492 | 12971 | 0.520 | 0.99 | | | |
| 5.00E−03 | 0.89 | 1712 | 0.506 | 12965 | 0.533 | 0.99 | | | |
| 5.18E−03 | 0.90 | 1715 | 0.507 | 12970 | 0.535 | 1.00 | | | |
| 5.33E−03 | 0.90 | 1713 | 0.493 | 12972 | 0.520 | 0.99 | | | |
| 5.63E−03 | 1.12 | 1713 | 0.490 | 12970 | 0.463 | 1.00 | | | |
| 5.70E−03 | 1.12 | 1713 | 0.411 | 12971 | 0.384 | 0.99 | | | |
| 6.05E−03 | 0.90 | 1715 | 0.507 | 12971 | 0.534 | 1.00 | | | |
| 6.60E−03 | 0.90 | 1705 | 0.510 | 12922 | 0.537 | 1.00 | | | |
| 6.60E−03 | 1.11 | 1705 | 0.490 | 12922 | 0.463 | 1.00 | | | |
| 6.69E−03 | 0.89 | 1715 | 0.614 | 12967 | 0.641 | 0.94 | 1 | | |
| 6.77E−03 | 1.12 | 1715 | 0.536 | 12971 | 0.509 | 0.95 | | | 4 |
| 6.90E−03 | 0.90 | 1715 | 0.496 | 12970 | 0.523 | 0.98 | 1 | | |
| 7.04E−03 | 0.90 | 1713 | 0.574 | 12967 | 0.600 | 1.00 | | | |
| 7.04E−03 | 1.11 | 1713 | 0.426 | 12967 | 0.400 | 1.00 | | | |
| 7.10E−03 | 1.12 | 1715 | 0.488 | 12972 | 0.461 | 0.92 | | 4 | |
| 7.50E−03 | 0.90 | 1715 | 0.509 | 12970 | 0.535 | 0.99 | | | |
| 9.23E−03 | 1.11 | 1713 | 0.409 | 12972 | 0.384 | 0.99 | | | |
| 9.69E−03 | 0.90 | 1715 | 0.509 | 12972 | 0.535 | 0.98 | | | |
| 1.04E−02 | 0.84 | 1715 | 0.117 | 12973 | 0.136 | 0.83 | | 2 | |
| 1.07E−02 | 0.85 | 1715 | 0.124 | 12972 | 0.143 | 0.83 | | 2 | |
| 1.09E−02 | 0.90 | 1715 | 0.509 | 12971 | 0.535 | 0.99 | | | |
| 1.13E−02 | 0.90 | 1715 | 0.516 | 12972 | 0.541 | 0.99 | | | |
| 1.20E−02 | 0.90 | 1711 | 0.498 | 12952 | 0.523 | 0.98 | 1 | | |
| 1.47E−02 | 0.90 | 1714 | 0.257 | 12967 | 0.279 | 0.98 | | | 3 |
| 1.75E−02 | 0.90 | 1715 | 0.267 | 12970 | 0.288 | 1.00 | | 2 | 3 |
| 1.80E−02 | 0.90 | 1713 | 0.267 | 12919 | 0.288 | 1.00 | | | 3 |
| 1.80E−02 | 1.11 | 1713 | 0.733 | 12919 | 0.712 | 1.00 | | | 4 |
| 1.85E−02 | 0.90 | 1714 | 0.266 | 12958 | 0.287 | 1.00 | 1 | | 3 |
| 1.87E−02 | 0.91 | 1715 | 0.535 | 12973 | 0.558 | 0.97 | | | |
| 1.92E−02 | 0.90 | 1715 | 0.268 | 12971 | 0.289 | 1.00 | 1 | 2 | |
| 1.96E−02 | 0.91 | 1715 | 0.522 | 12973 | 0.545 | 0.96 | | | |
| 2.25E−02 | 0.90 | 1714 | 0.231 | 12972 | 0.251 | 0.95 | | | 3 |
| 2.47E−02 | 0.90 | 1714 | 0.231 | 12962 | 0.251 | 0.95 | | | 3 |
| 2.59E−02 | 0.90 | 1714 | 0.228 | 12971 | 0.248 | 0.93 | | | 3 |
| 2.89E−02 | 0.81 | 1715 | 0.027 | 12971 | 0.033 | 1.40 | | | 3 |
| 3.33E−02 | 0.79 | 1715 | 0.022 | 12972 | 0.027 | 1.40 | | | 3 |
| 3.38E−02 | 3.50 | 1713 | 0.002 | 12970 | 0.001 | 0.99 | | | |
| 3.41E−02 | 0.87 | 1715 | 0.108 | 12967 | 0.122 | 0.87 | 2 | | |
| 4.73E−02 | 0.92 | 1715 | 0.529 | 12973 | 0.549 | 0.95 | 1 | | |
| 5.76E−02 | 0.85 | 1715 | 0.043 | 12973 | 0.050 | 1.26 | 2 | | |
| 6.49E−02 | 0.92 | 1715 | 0.244 | 12972 | 0.261 | 0.95 | | | 3 |
| 6.73E−02 | 0.88 | 1715 | 0.098 | 12967 | 0.109 | 0.94 | 2 | | |
| 7.04E−02 | 0.89 | 1715 | 0.097 | 12971 | 0.108 | 0.94 | 2 | | |

| p-val | rs1420533 | rs4783780 | rs3803662 | rs3104755 | rs4784230 | rs3112612 |
|---|---|---|---|---|---|---|
| 2.46E−06 | 1 | | | | 4 | |
| 4.35E−06 | 1 | | | 1 | | |
| 5.16E−06 | | | 4 | | | |
| 6.12E−06 | | | 4 | | 4 | |
| 6.91E−06 | | 2 | | | 4 | |
| 7.39E−06 | | | 4 | | | |
| 8.18E−06 | | 2 | | 1 | | |
| 8.36E−06 | | | | | 4 | 4 |
| 9.13E−06 | 1 | | 4 | | | |
| 9.86E−06 | | | 4 | | | 4 |
| 1.06E−05 | | | 4 | | | |
| 1.08E−05 | | | 2 | | | |
| 1.08E−05 | | | 4 | | | |
| 1.10E−05 | | | 4 | 1 | | |
| 1.30E−05 | | 2 | 4 | | | |
| 2.67E−05 | | | | 1 | | 4 |
| 3.31E−05 | 1 | | | | | |
| 6.62E−05 | | 2 | | | | |
| 1.04E−04 | 1 | | | | | |
| 1.05E−04 | | | | | | 4 |
| 2.30E−04 | | 2 | | | | |
| 5.78E−04 | | | 2 | | | |
| 1.24E−03 | | | | | | 4 |
| 1.39E−03 | 1 | | | | | |
| 1.89E−03 | | | | | | 4 |
| 1.99E−03 | | 2 | | | | |
| 2.01E−03 | | | | | | |
| 2.19E−03 | | | | | | |
| 2.36E−03 | | | | | | |
| 2.36E−03 | | | | | | |
| 2.38E−03 | | | | | | |
| 2.67E−03 | | | | | 4 | |
| 3.37E−03 | | | | | | 2 |
| 3.43E−03 | | 1 | | | | |
| 3.44E−03 | | | | | 4 | |
| 3.45E−03 | 3 | | | | | |
| 3.64E−03 | | | 2 | | | 2 |
| 4.13E−03 | 1 | | | | | |
| 4.13E−03 | 3 | | | | | |
| 4.51E−03 | 3 | 1 | | | | |
| 4.58E−03 | 3 | | 2 | | | |
| 4.71E−03 | 3 | | | | | 2 |
| 5.00E−03 | | 1 | 2 | | | |
| 5.18E−03 | 3 | | | 1 | | |
| 5.33E−03 | | 1 | | | | 2 |
| 5.63E−03 | 1 | 2 | | | | |
| 5.70E−03 | 1 | | | | | 4 |
| 6.05E−03 | 3 | | | | 4 | |
| 6.60E−03 | | 1 | | | | |
| 6.60E−03 | | 2 | | | | |
| 6.69E−03 | | | 2 | | | |
| 6.77E−03 | | | | 1 | | |
| 6.90E−03 | 3 | | | | | |
| 7.04E−03 | | | | | | 2 |
| 7.04E−03 | | | | | | 4 |
| 7.10E−03 | | | | 1 | | |
| 7.50E−03 | | 1 | | 1 | | |
| 9.23E−03 | | 2 | | | | 4 |
| 9.69E−03 | | | 2 | 1 | | |
| 1.04E−02 | | | | | 2 | |
| 1.07E−02 | | | | 3 | | |
| 1.09E−02 | | 1 | | | 4 | |
| 1.13E−02 | | | 2 | | 4 | |
| 1.20E−02 | | 1 | | | | |
| 1.47E−02 | | | 2 | | | |
| 1.75E−02 | | | | | | |
| 1.80E−02 | | | | | | |
| 1.80E−02 | | | | | | |
| 1.85E−02 | | | | | | |
| 1.87E−02 | | | | | 4 | 2 |
| 1.92E−02 | | | | | | |
| 1.96E−02 | | | | 1 | | 2 |
| 2.25E−02 | 3 | | | | | |
| 2.47E−02 | | 1 | | | | |
| 2.59E−02 | | | | | | 2 |
| 2.89E−02 | | | | 3 | | |
| 3.33E−02 | | | | | 2 | |
| 3.38E−02 | 1 | 1 | | | | |
| 3.41E−02 | | | 2 | | | |
| 4.73E−02 | | | | | | 2 |
| 5.76E−02 | | | | | | 2 |
| 6.49E−02 | | | | | 4 | |
| 6.73E−02 | | | | 3 | | |
| 7.04E−02 | | | | | 2 | |

TABLE 19
HapMap SNP markers that are correlated with
rs3803662 with an r² value greater than 0.2:

| Surrogate | | | | | Pos in | Pos in | Pos in |
|---|---|---|---|---|---|---|---|
| SNP | Ref SNP | D′ | R2 | p-value | Build 34 | Build 36 | SEQ ID 6 |
| rs4784220 | rs3803662 | 0.675773 | 0.314847 | 8.65E−09 | 52314403 | 51093311 | 301 |
| rs12598982 | rs3803662 | 0.939522 | 0.331013 | 2.84E−11 | 52315478 | 51094386 | 1376 |
| rs4784222 | rs3803662 | 0.93875 | 0.331747 | 4.06E−11 | 52316179 | 51095087 | 2077 |
| rs17271951 | rs3803662 | 0.959839 | 0.88572 | 9.27E−27 | 52316633 | 51095541 | 2531 |
| rs9933638 | rs3803662 | 0.935921 | 0.321096 | 2.07E−10 | 52318190 | 51097098 | 4088 |
| rs9302556 | rs3803662 | 0.936406 | 0.334336 | 1.07E−10 | 52322238 | 51101146 | 8136 |
| rs7190749 | rs3803662 | 0.939522 | 0.331013 | 2.84E−11 | 52322671 | 51101579 | 8569 |
| rs12443621 | rs3803662 | 0.939123 | 0.326882 | 4.01E−11 | 52326630 | 51105538 | 12528 |
| rs9933556 | rs3803662 | 0.937324 | 0.325388 | 9.50E−11 | 52326895 | 51105803 | 12793 |
| rs1362546 | rs3803662 | 0.935967 | 0.329172 | 9.67E−11 | 52328528 | 51107436 | 14426 |
| rs1075367 | rs3803662 | 0.939522 | 0.331013 | 2.84E−11 | 52333595 | 51112503 | 19493 |
| rs8046979 | rs3803662 | 0.932586 | 0.342705 | 3.56E−10 | 52334335 | 51113243 | 20233 |
| rs1420529 | rs3803662 | 0.931004 | 0.310285 | 1.29E−09 | 52337266 | 51116174 | 23164 |
| rs11642645 | rs3803662 | 0.936189 | 0.329297 | 5.09E−11 | 52337500 | 51116408 | 23398 |
| rs1420533 | rs3803662 | 1 | 0.362637 | 3.83E−14 | 52342219 | 51121127 | 28117 |
| rs1362548 | rs3803662 | 1 | 0.924812 | 8.14E−30 | 52342544 | 51121452 | 28442 |
| rs2193094 | rs3803662 | 1 | 0.362637 | 3.83E−14 | 52345047 | 51123955 | 30945 |
| rs4783780 | rs3803662 | 1 | 0.362637 | 3.83E−14 | 52350025 | 51128937 | 35927 |
| rs3112581 | rs3803662 | 1 | 0.358407 | 5.51E−14 | 52350122 | 51129034 | 36024 |
| rs3112580 | rs3803662 | 1 | 0.370787 | 2.40E−14 | 52350194 | 51129106 | 36096 |
| rs9931232 | rs3803662 | 1 | 0.362637 | 3.83E−14 | 52351417 | 51130333 | 37323 |
| rs1123428 | rs3803662 | 1 | 0.366516 | 3.46E−14 | 52356420 | 51135336 | 42326 |
| rs3095604 | rs3803662 | 1 | 1 | 9.56E−34 | 52360562 | 51139480 | 46470 |
| rs3803662 | rs3803662 | 1 | 1 | 0 | 52364943 | 51143842 | 50832 |
| rs4784227 | rs3803662 | 1 | 0.812734 | 3.45E−25 | 52377790 | 51156689 | 63679 |
| rs12922061 | rs3803662 | 0.813366 | 0.393164 | 1.28E−08 | 52413602 | 51192501 | 99491 |

TABLE 20
List of all dbSNP125 markers in the rs4848543 Linkage Disequilibrium
block (positions are from NCBI Build 34):

| Pos Build | |
|---|---|
| 34 | Name |
| 119987193 | rs11883887 |
| 119987218 | rs7601379 |
| 119987236 | rs10189928 |
| 119987488 | rs17012230 |
| 119987701 | rs17798163 |
| 119988214 | rs6542503 |
| 119988417 | rs7606204 |
| 119988662 | rs17012243 |
| 119988746 | rs10174027 |
| 119988777 | rs17012263 |
| 119988845 | rs12623644 |
| 119988905 | rs10184937 |
| 119989602 | rs7570704 |
| 119989690 | rs7573686 |
| 119990027 | rs6542504 |
| 119990110 | rs7557802 |
| 119990364 | rs12987676 |
| 119990863 | rs12993619 |
| 119991237 | rs6737956 |
| 119991499 | rs10529569 |
| 119991618 | rs6741215 |
| 119991739 | rs11677153 |
| 119991908 | rs6542505 |
| 119991993 | rs6728751 |
| 119992039 | rs6542506 |
| 119992209 | rs11386094 |
| 119993577 | rs6721529 |
| 119993825 | rs6739574 |
| 119993914 | rs12711922 |
| 119993983 | rs13397501 |
| 119994264 | rs12711923 |
| 119994321 | rs13017443 |
| 119994574 | rs6740231 |
| 119994784 | rs10716622 |
| 119994803 | rs1374121 |
| 119994954 | rs1530522 |
| 119995078 | rs10205049 |
| 119995182 | rs1530523 |
| 119995238 | rs5018771 |
| 119995420 | rs13417283 |
| 119995638 | rs2860753 |
| 119995975 | rs4447611 |
| 119996042 | rs4485581 |
| 119996150 | rs10864979 |
| 119996950 | rs13433007 |
| 119996967 | rs6748754 |
| 119997458 | rs28574898 |
| 119998119 | rs4848537 |
| 119998463 | rs13409086 |
| 119998504 | rs13411872 |
| 119998551 | rs13420759 |
| 119999028 | rs11895163 |
| 119999114 | rs7597287 |
| 120000098 | rs13020121 |
| 120000215 | rs9308770 |
| 120000292 | rs13425098 |
| 120000559 | rs895404 |
| 120000798 | rs10207133 |
| 120000837 | rs10207198 |
| 120000969 | rs11898182 |
| 120001134 | rs9308771 |
| 120001139 | rs9308772 |
| 120001461 | rs6146899 |
| 120001581 | rs17012317 |
| 120001748 | rs9308773 |
| 120001768 | rs9308774 |
| 120002407 | rs10202747 |
| 120002993 | rs17190912 |
| 120003189 | rs11884636 |
| 120003199 | rs4849760 |
| 120003758 | rs7562612 |
| 120004530 | rs745884 |
| 120004532 | rs745885 |
| 120004821 | rs745886 |
| 120005202 | rs745887 |
| 120005459 | rs745888 |
| 120005770 | rs13395242 |
| 120005783 | rs7560869 |
| 120005868 | rs11885255 |
| 120005995 | rs17012332 |
| 120006051 | rs11900137 |
| 120006067 | rs11884243 |
| 120006186 | rs2197538 |
| 120006239 | rs11889299 |
| 120006306 | rs970013 |
| 120007130 | rs7562248 |
| 120007189 | rs12468740 |
| 120007959 | rs6542507 |
| 120008874 | rs4848538 |
| 120009246 | rs6758051 |
| 120010134 | rs7593041 |
| 120010278 | rs11308896 |
| 120010394 | rs7556873 |
| 120010590 | rs3980309 |
| 120010592 | rs3980308 |
| 120010654 | rs1317848 |
| 120010949 | rs4643545 |
| 120011133 | rs6705476 |
| 120011458 | rs2121217 |
| 120011699 | rs5833777 |
| 120011761 | rs7600045 |
| 120012134 | rs7563952 |
| 120012410 | rs4849761 |
| 120012531 | rs1446130 |
| 120012783 | rs1446129 |
| 120013821 | rs4849762 |
| 120014233 | rs6542508 |
| 120014416 | rs11299793 |
| 120014424 | rs6724034 |
| 120014425 | rs6706020 |
| 120014553 | rs6542509 |
| 120014683 | rs6542510 |
| 120015600 | rs1867750 |
| 120015668 | rs5019100 |
| 120015928 | rs1867751 |
| 120016277 | rs7420091 |
| 120016397 | rs4390775 |
| 120016788 | rs6732650 |
| 120016904 | rs6742613 |
| 120017026 | rs7579840 |
| 120017542 | rs2197539 |
| 120017573 | rs10205385 |
| 120017765 | rs6542511 |
| 120018695 | rs6716071 |
| 120018841 | rs10597874 |
| 120019534 | rs13407241 |
| 120019716 | rs4849763 |
| 120019836 | rs4848539 |
| 120019839 | rs4848540 |
| 120019852 | rs1867752 |
| 120020267 | rs10531839 |
| 120020619 | rs2197540 |
| 120020703 | rs6727359 |
| 120020806 | rs2197541 |
| 120020814 | rs6737324 |
| 120020831 | rs2197542 |
| 120021103 | rs7577922 |
| 120021157 | rs7575097 |
| 120021526 | rs6713238 |
| 120021548 | rs7564432 |
| 120021672 | rs11682799 |
| 120021695 | rs11687834 |
| 120022550 | rs2121218 |
| 120022556 | rs2121219 |
| 120022644 | rs13418186 |
| 120022871 | rs2166408 |
| 120022882 | rs21 66409 |
| 120022986 | rs6732687 |
| 120023118 | rs12474874 |
| 120023404 | rs6748897 |
| 120023422 | rs4322838 |
| 120023516 | rs2901763 |
| 120023583 | rs11680207 |
| 120024083 | rs6722032 |
| 120024822 | rs7592528 |
| 120024900 | rs6542512 |
| 120025087 | rs7371346 |
| 120025150 | rs10864980 |
| 120026424 | rs10188216 |
| 120026569 | rs2044428 |
| 120026637 | rs11123503 |
| 120026789 | rs2044427 |
| 120027283 | rs10203658 |
| 120027724 | rs7583824 |
| 120028226 | rs10174654 |
| 120028314 | rs10186930 |
| 120028820 | rs6542513 |
| 120028843 | rs6542514 |
| 120029239 | rs6542515 |
| 120029586 | rs10532262 |
| 120029885 | rs4849764 |
| 120030487 | rs17804511 |
| 120030834 | rs935362 |
| 120030947 | rs935361 |
| 120031161 | rs2860752 |
| 120031486 | rs11893808 |
| 120031828 | rs12623976 |
| 120032169 | rs4848541 |
| 120032367 | rs28653619 |
| 120032378 | rs1446128 |
| 120033081 | rs1838763 |
| 120033129 | rs1470474 |
| 120033135 | rs4528761 |
| 120033150 | rs1838762 |
| 120033268 | rs2121216 |
| 120033353 | rs3980311 |
| 120033359 | rs1446127 |
| 120033749 | rs13024710 |
| 120034173 | rs4849765 |
| 120034354 | rs4849766 |
| 120034730 | rs6715243 |
| 120036029 | rs10166010 |
| 120036225 | rs7561309 |
| 120036417 | rs11886602 |
| 120036954 | rs6542516 |
| 120037241 | rs7601541 |
| 120037242 | rs7562256 |
| 120037469 | rs7557444 |
| 120037538 | rs6735202 |
| 120037631 | rs7584874 |
| 120037714 | rs7584975 |
| 120037998 | rs7598853 |
| 120038031 | rs11123504 |
| 120038363 | rs11384356 |
| 120038373 | rs11464659 |
| 120038374 | rs11393319 |
| 120038698 | rs1446126 |
| 120038699 | rs10168744 |
| 120039011 | rs4848542 |
| 120039313 | rs4277535 |
| 120039785 | rs13416713 |
| 120040375 | rs13417356 |
| 120040703 | rs12614389 |
| 120040851 | rs2084154 |
| 120040967 | rs12621053 |
| 120041245 | rs10167806 |
| 120041777 | rs10641092 |
| 120041779 | rs895400 |
| 120041797 | rs7599963 |
| 120041798 | rs10658512 |
| 120041840 | rs7560645 |
| 120041953 | rs10551390 |
| 120041983 | rs7600179 |
| 120042110 | rs13017664 |
| 120042545 | rs10206284 |
| 120042980 | rs3053193 |
| 120042985 | rs5833778 |
| 120043240 | rs13024716 |
| 120044181 | rs7607939 |
| 120044182 | rs13405317 |
| 120044735 | rs13020459 |
| 120045023 | rs13015999 |
| 120045027 | rs12052957 |
| 120045116 | rs12468083 |
| 120045623 | rs963662 |
| 120046099 | rs12472931 |
| 120046932 | rs7579602 |
| 120047249 | rs5833779 |
| 120047386 | rs13030441 |
| 120047590 | rs991844 |
| 120048218 | rs13000062 |
| 120048226 | rs13022674 |
| 120048234 | rs28579959 |
| 120048244 | rs28719630 |
| 120048434 | rs13027917 |
| 120048457 | rs13027932 |
| 120048632 | rs12466475 |
| 120049031 | rs4849767 |
| 120049111 | rs4849768 |
| 120049158 | rs4849769 |
| 120049468 | rs13017938 |
| 120049808 | rs11680905 |
| 120049895 | rs2028852 |
| 120050448 | rs2028851 |
| 120050503 | rs13410792 |
| 120050761 | rs13402151 |
| 120050907 | rs7586792 |
| 120051111 | rs11675684 |
| 120051961 | rs4142753 |
| 120052171 | rs4849770 |
| 120052802 | rs2084155 |
| 120053731 | rs11691024 |
| 120054452 | rs11681612 |
| 120054477 | rs11692475 |
| 120054484 | rs13400379 |
| 120054490 | rs11692479 |
| 120055083 | rs4142752 |
| 120056573 | rs11695796 |
| 120057058 | rs10188745 |
| 120057116 | rs11685085 |
| 120057332 | rs12475875 |
| 120057333 | rs12463432 |
| 120057493 | rs5833780 |
| 120057623 | rs746945 |
| 120057815 | rs5833781 |
| 120057816 | rs3053192 |
| 120057829 | rs5833782 |
| 120058071 | rs876062 |
| 120058160 | rs895399 |
| 120058174 | rs10186657 |
| 120058180 | rs895398 |
| 120058601 | rs5833783 |
| 120060148 | rs10181114 |
| 120060448 | rs2860751 |
| 120060852 | rs11693876 |
| 120061012 | rs11683050 |
| 120061029 | rs10184332 |
| 120061096 | rs4848543 |
| 120061139 | rs10184438 |
| 120061708 | rs11123505 |
| 120062319 | rs11684731 |
| 120062891 | rs6754436 |
| 120062996 | rs12464139 |
| 120063070 | rs6754664 |
| 120063176 | rs6754799 |
| 120063963 | rs12997936 |
| 120064284 | rs10206700 |
| 120064289 | rs10194572 |
| 120064976 | rs6759589 |
| 120065127 | rs10221610 |
| 120065310 | rs11689491 |
| 120066060 | rs7560972 |
| 120066128 | rs7558272 |
| 120066241 | rs12463977 |
| 120066318 | rs1562256 |
| 120066453 | rs1562255 |
| 120066620 | rs1446125 |
| 120066705 | rs1446124 |
| 120066972 | rs1838761 |
| 120066974 | rs7601127 |
| 120067290 | rs11677262 |
| 120067736 | rs10204391 |
| 120067927 | rs6753314 |
| 120068051 | rs6735649 |
| 120068477 | rs28455610 |
| 120068761 | rs6709777 |
| 120069635 | rs10208455 |
| 120069642 | rs838067 |
| 120069925 | rs4848544 |
| 120070154 | rs12466050 |
| 120070400 | rs7565771 |
| 120070666 | rs7595954 |
| 120070908 | rs708666 |
| 120071508 | rs838066 |
| 120071996 | rs708667 |
| 120072367 | rs708668 |
| 120072376 | rs708669 |
| 120072400 | rs895397 |
| 120072476 | rs838065 |
| 120072631 | rs6755420 |
| 120072650 | rs838064 |
| 120072686 | rs838063 |
| 120072737 | rs838062 |
| 120073503 | rs838061 |
| 120073744 | rs11683528 |
| 120073904 | rs10209847 |
| 120074221 | rs1562310 |
| 120074355 | rs838060 |
| 120074581 | rs7557910 |
| 120075395 | rs838059 |
| 120075418 | rs838058 |
| 120076234 | rs11676144 |
| 120076668 | rs2289894 |
| 120076919 | rs13003725 |
| 120076920 | rs13034799 |
| 120077150 | rs3795927 |
| 120077389 | rs3795926 |
| 120077721 | rs838112 |
| 120077754 | rs838111 |
| 120077769 | rs838110 |
| 120077991 | rs838109 |
| 120078174 | rs838108 |
| 120078320 | rs12373823 |
| 120078344 | rs838107 |
| 120078395 | rs865688 |
| 120078483 | rs12373677 |
| 120078615 | rs7578870 |
| 120078754 | rs838106 |
| 120079222 | rs838105 |
| 120079745 | rs838104 |
| 120079819 | rs838103 |
| 120079878 | rs838102 |
| 120080519 | rs10182677 |
| 120080542 | rs10625730 |
| 120081128 | rs3769659 |
| 120081529 | rs865108 |
| 120082110 | rs838101 |
| 120082382 | rs708670 |
| 120082432 | rs708671 |
| 120082499 | rs708672 |
| 120082585 | rs6146900 |
| 120082871 | rs708673 |
| 120083300 | rs1106771 |
| 120083710 | rs838100 |
| 120084104 | rs3223038 |
| 120084105 | rs6146901 |
| 120084215 | rs838099 |
| 120084226 | rs838098 |
| 120084235 | rs13410686 |
| 120084331 | rs3769656 |
| 120084565 | rs838097 |
| 120084736 | rs13400593 |
| 120084741 | rs838096 |
| 120084781 | rs838095 |
| 120085360 | rs13425946 |
| 120085486 | rs838094 |
| 120085502 | rs13010169 |
| 120085910 | rs12470404 |
| 120086734 | rs895396 |
| 120086898 | rs5833784 |
| 120086917 | rs13432674 |
| 120086997 | rs13408034 |
| 120087374 | rs708674 |
| 120087538 | rs2121358 |
| 120087544 | rs1963247 |
| 120087718 | rs12465926 |
| 120088448 | rs7577784 |
| 120088805 | rs13391380 |
| 120089829 | rs838093 |
| 120090797 | rs10190429 |
| 120090847 | rs10182241 |
| 120091298 | rs838092 |
| 120091323 | rs13401854 |
| 120091399 | rs13386059 |
| 120091931 | rs838091 |
| 120092184 | rs864505 |
| 120092304 | rs838090 |
| 120092610 | rs7567193 |
| 120092737 | rs838089 |
| 120092937 | rs838088 |
| 120093034 | rs10174436 |
| 120093360 | rs838087 |
| 120094306 | rs12467944 |
| 120095038 | rs17804991 |
| 120095312 | rs10196144 |
| 120095426 | rs13027072 |
| 120095756 | rs6728660 |
| 120096263 | rs13027293 |
| 120096647 | rs838086 |
| 120096791 | rs838085 |
| 120096949 | rs10600943 |
| 120096982 | rs10526493 |
| 120097896 | rs10196222 |
| 120097915 | rs838084 |
| 120097952 | rs3731606 |
| 120098787 | rs838083 |
| 120098925 | rs10188946 |
| 120099084 | rs838082 |
| 120099515 | rs838081 |
| 120099779 | rs10202732 |
| 120099996 | rs6738309 |
| 120100457 | rs17013371 |
| 120100570 | rs1665025 |
| 120100571 | rs1624263 |
| 120100714 | rs17805141 |
| 120100985 | rs5833785 |
| 120100993 | rs1530524 |
| 120101012 | rs4592854 |
| 120101809 | rs2592075 |
| 120102502 | rs838080 |
| 120102508 | rs6735209 |
| 120102857 | rs838079 |
| 120103067 | rs17013379 |
| 120103130 | rs12990907 |
| 120103529 | rs7561146 |
| 120103701 | rs734846 |
| 120103880 | rs734845 |
| 120104365 | rs895406 |
| 120104472 | rs895405 |
| 120104502 | rs708675 |
| 120104672 | rs1867856 |
| 120105219 | rs838078 |
| 120105720 | rs3754847 |
| 120105721 | rs12467089 |
| 120105810 | rs11888609 |
| 120105981 | rs838077 |
| 120106044 | rs7596511 |
| 120106433 | rs838076 |
| 120107316 | rs12711924 |
| 120108033 | rs838075 |
| 120108409 | rs838074 |
| 120109057 | rs1867749 |
| 120109148 | rs12104548 |
| 120109236 | rs1045623 |
| 120109381 | rs3731604 |
| 120109485 | rs12104639 |
| 120109754 | rs838073 |
| 120109782 | rs12619779 |
| 120109902 | rs6742425 |
| 120110130 | rs838072 |
| 120110342 | rs6542517 |
| 120110521 | rs838071 |
| 120111317 | rs12991476 |
| 120111319 | rs12991477 |
| 120111544 | rs12105674 |
| 120111606 | rs838070 |
| 120111771 | rs838069 |
| 120111887 | rs838068 |
| 120112630 | rs13400106 |
| 120112767 | rs13388672 |
| 120112793 | rs7575387 |
| 120113041 | rs1610469 |
| 120113297 | rs11891374 |
| 120114676 | rs6720040 |
| 120114755 | rs6723169 |
| 120115056 | rs3054837 |
| 120115077 | rs5833786 |
| 120115093 | rs13014052 |
| 120115134 | rs1374314 |
| 120115780 | rs11885203 |
| 120116464 | rs3769653 |
| 120116484 | rs10171408 |
| 120116695 | rs7590206 |
| 120116930 | rs7581339 |
| 120117062 | rs3731603 |
| 120117527 | rs6753005 |
| 120117530 | rs6753006 |
| 120117532 | rs12476967 |
| 120117536 | rs10571222 |
| 120117768 | rs10204553 |
| 120118122 | rs3731602 |
| 120118333 | rs4849771 |
| 120118405 | rs11694139 |
| 120118612 | rs11682650 |
| 120119587 | rs1530561 |
| 120119648 | rs6721852 |
| 120119899 | rs13016861 |
| 120120260 | rs13034803 |
| 120120268 | rs13034810 |
| 120120378 | rs13012570 |
| 120120617 | rs6707849 |
| 120120725 | rs12165162 |
| 120121081 | rs6723312 |
| 120121286 | rs3054836 |
| 120122044 | rs1545360 |
| 120122260 | rs6740573 |
| 120122331 | rs6730921 |
| 120122555 | rs13004570 |
| 120122566 | rs5028235 |
| 120122626 | rs11395093 |
| 120123526 | rs2860792 |
| 120123689 | rs1867855 |
| 120123862 | rs5833787 |
| 120124074 | rs28497293 |
| 120124123 | rs12990281 |
| 120124221 | rs12995866 |
| 120125144 | rs11692352 |
| 120125454 | rs13402922 |
| 120126304 | rs6729196 |
| 120126843 | rs13034935 |
| 120127261 | rs7599282 |
| 120127470 | rs7582316 |
| 120127897 | rs11123506 |
| 120128212 | rs2084201 |
| 120128319 | rs7592522 |
| 120128879 | rs10864981 |
| 120128983 | rs11123507 |

TABLE 21
List of all dbSNP125 markers in the rs13387042 Linkage Disequilibrium
block (positions are from NCBI Build 34):

| Pos in | |
|---|---|
| Build 34 | Name |
| 218062199 | rs10191184 |
| 218063010 | rs4396687 |
| 218063034 | rs5838616 |
| 218063341 | rs10932687 |
| 218063429 | rs12614802 |
| 218063508 | rs12614767 |
| 218063667 | rs11889406 |
| 218063784 | rs16856807 |
| 218064064 | rs2888449 |
| 218064289 | rs10192649 |
| 218064351 | rs4571035 |
| 218064664 | rs2372931 |
| 218065238 | rs10195963 |
| 218065423 | rs17833456 |
| 218065966 | rs10207650 |
| 218066023 | rs10932688 |
| 218066050 | rs13403428 |
| 218066096 | rs10207736 |
| 218066454 | rs28663434 |
| 218067143 | rs12464018 |
| 218067861 | rs16856812 |
| 218068232 | rs12477491 |
| 218068256 | rs12477493 |
| 218068532 | rs10932689 |
| 218068574 | rs13413746 |
| 218068580 | rs10932690 |
| 218068673 | rs16856816 |
| 218068844 | rs13414233 |
| 218068906 | rs12613030 |
| 218069197 | rs11903648 |
| 218069383 | rs2888450 |
| 218069406 | rs11688968 |
| 218069916 | rs4994976 |
| 218069959 | rs4254482 |
| 218069975 | rs2372932 |
| 218070046 | rs6723386 |
| 218070106 | rs6435956 |
| 218070227 | rs6713022 |
| 218070431 | rs4255939 |
| 218070433 | rs2372933 |
| 218070638 | rs10200456 |
| 218071353 | rs7426087 |
| 218071353 | rs4542823 |
| 218071539 | rs4456677 |
| 218071539 | rs7422519 |
| 218071631 | rs4264545 |
| 218071948 | rs10169454 |
| 218073196 | rs10192415 |
| 218073796 | rs7595393 |
| 218073891 | rs10169372 |
| 218074854 | rs13430080 |
| 218075719 | rs10175911 |
| 218075836 | rs12165215 |
| 218076028 | rs17833745 |
| 218076072 | rs2372934 |
| 218076131 | rs2372935 |
| 218076869 | rs12612444 |
| 218076958 | rs13423850 |
| 218077438 | rs6733648 |
| 218077473 | rs13389571 |
| 218077681 | rs12165173 |
| 218077715 | rs13389783 |
| 218078368 | rs6737508 |
| 218078384 | rs10209168 |
| 218079285 | rs13383189 |
| 218079764 | rs11884129 |
| 218080177 | rs6714036 |
| 218080751 | rs6435957 |
| 218081124 | rs10192982 |
| 218082238 | rs10932691 |
| 218082405 | rs10932692 |
| 218082754 | rs10188365 |
| 218083373 | rs6723019 |
| 218083726 | rs7349199 |
| 218083877 | rs13404341 |
| 218084046 | rs16856821 |
| 218084485 | rs12614773 |
| 218086136 | rs6732152 |
| 218086701 | rs11677001 |
| 218086856 | rs10171745 |
| 218087663 | rs17833842 |
| 218087886 | rs13393574 |
| 218087900 | rs13406843 |
| 218088071 | rs10198696 |
| 218088201 | rs10177578 |
| 218088354 | rs10177962 |
| 218089512 | rs4435428 |
| 218089563 | rs4276003 |
| 218089745 | rs13425828 |
| 218089919 | rs3084560 |
| 218089928 | rs13414201 |
| 218090342 | rs12623304 |
| 218091057 | rs2372936 |
| 218092002 | rs7425648 |
| 218092308 | rs13410769 |
| 218092732 | rs10191536 |
| 218092782 | rs10203987 |
| 218093185 | rs6716542 |
| 218093638 | rs10207757 |
| 218093795 | rs10173250 |
| 218094186 | rs28525300 |
| 218094234 | rs17777330 |
| 218094822 | rs10211354 |
| 218094838 | rs10187690 |
| 218094913 | rs10211289 |
| 218094939 | rs10211546 |
| 218095256 | rs2372937 |
| 218095412 | rs4315498 |
| 218096305 | rs12998806 |
| 218096485 | rs12999224 |
| 218096566 | rs17833945 |
| 218096592 | rs12465396 |
| 218096776 | rs12465506 |
| 218096945 | rs12465515 |
| 218097298 | rs4491709 |
| 218097359 | rs4674130 |
| 218097448 | rs3084573 |
| 218097540 | rs13417588 |
| 218097624 | rs13392238 |
| 218097651 | rs2372938 |
| 218098168 | rs12621130 |
| 218098897 | rs11387578 |
| 218099133 | rs4471865 |
| 218099154 | rs6435958 |
| 218099578 | rs6722681 |
| 218100083 | rs13399995 |
| 218100630 | rs7564605 |
| 218100783 | rs12052807 |
| 218101273 | rs10199394 |
| 218101451 | rs10210524 |
| 218101477 | rs2372939 |
| 218101566 | rs2372940 |
| 218101794 | rs4583440 |
| 218101850 | rs2372941 |
| 218101894 | rs10187530 |
| 218101902 | rs6760041 |
| 218102145 | rs10190181 |
| 218102201 | rs10190349 |
| 218102294 | rs17834221 |
| 218102294 | rs10202715 |
| 218102377 | rs6705818 |
| 218102717 | rs6706491 |
| 218102822 | rs6735174 |
| 218102838 | rs6721811 |
| 218102872 | rs6735190 |
| 218102939 | rs6735298 |
| 218103034 | rs17777767 |
| 218103042 | rs12464375 |
| 218103061 | rs12476702 |
| 218103151 | rs6435959 |
| 218103234 | rs6435960 |
| 218103592 | rs2372942 |
| 218104122 | rs4594417 |
| 218104281 | rs10709272 |
| 218104345 | rs12615418 |
| 218104452 | rs12621884 |
| 218104650 | rs7572103 |
| 218104906 | rs7421376 |
| 218105047 | rs11681010 |
| 218105186 | rs5838617 |
| 218105193 | rs3084579 |
| 218105260 | rs5838618 |
| 218105268 | rs6711784 |
| 218105513 | rs9973728 |
| 218105657 | rs9973894 |
| 218105711 | rs9973730 |
| 218105920 | rs13402323 |
| 218106201 | rs5838619 |
| 218106318 | rs2372943 |
| 218106548 | rs4522583 |
| 218106783 | rs13431146 |
| 218107055 | rs2888451 |
| 218107332 | rs13383392 |
| 218107518 | rs2270398 |
| 218107985 | rs6734131 |
| 218108214 | rs13386831 |
| 218108244 | rs13412479 |
| 218108254 | rs13412482 |
| 218108321 | rs13412666 |
| 218108374 | rs13387042 |
| 218108601 | rs13426268 |
| 218108615 | rs13387273 |
| 218108788 | rs13426489 |
| 218108844 | rs13413328 |
| 218108906 | rs13413357 |
| 218108957 | rs13387717 |
| 218108958 | rs13426688 |
| 218109040 | rs13413523 |
| 218109091 | rs17777938 |
| 218109320 | rs13416240 |
| 218109630 | rs6713249 |
| 218109728 | rs13391022 |
| 218109746 | rs13416773 |
| 218109779 | rs13416605 |
| 218109902 | rs13430215 |
| 218109976 | rs13416831 |
| 218109979 | rs13416834 |
| 218110056 | rs12620095 |
| 218110145 | rs13433000 |
| 218110379 | rs12327908 |
| 218110607 | rs2372944 |
| 218110643 | rs2888452 |
| 218110863 | rs4618027 |
| 218111014 | rs10490444 |
| 218111297 | rs13397674 |
| 218111343 | rs13423473 |
| 218111527 | rs4621152 |
| 218111631 | rs13386262 |
| 218111703 | rs12329160 |
| 218111876 | rs12329163 |
| 218112005 | rs6721996 |
| 218112094 | rs12329172 |
| 218112403 | rs12622764 |
| 218112445 | rs10206095 |
| 218112816 | rs10206577 |
| 218113130 | rs17778091 |
| 218113325 | rs13411606 |
| 218113452 | rs6723013 |
| 218113636 | rs13386291 |
| 218113745 | rs10685531 |
| 218113820 | rs10192175 |
| 218113854 | rs10192253 |
| 218113962 | rs10192501 |
| 218114027 | rs10204872 |
| 218114030 | rs10192589 |
| 218114046 | rs6708579 |
| 218114076 | rs10192446 |
| 218114103 | rs10192609 |
| 218114176 | rs4672818 |
| 218114436 | rs10195111 |
| 218114560 | rs10171550 |
| 218114629 | rs10195322 |
| 218114644 | rs10195505 |
| 218114737 | rs10195441 |
| 218114786 | rs10207917 |
| 218115389 | rs10198604 |
| 218115494 | rs10198699 |
| 218115540 | rs10198621 |
| 218115617 | rs10174948 |
| 218115747 | rs10175133 |
| 218115929 | rs10198996 |
| 218116467 | rs10167002 |
| 218116654 | rs2372945 |
| 218116683 | rs10201966 |
| 218116715 | rs10201982 |
| 218116795 | rs10178467 |
| 218117052 | rs16856849 |
| 218117184 | rs10204819 |
| 218117267 | rs5838620 |
| 218117273 | rs10566949 |
| 218117273 | rs3084597 |
| 218117443 | rs11901909 |
| 218117759 | rs11901972 |
| 218117956 | rs12694403 |
| 218118356 | rs11892687 |
| 218118594 | rs12613955 |
| 218118809 | rs10208746 |
| 218119095 | rs10185401 |
| 218119709 | rs11894340 |
| 218119744 | rs6435961 |
| 218120136 | rs10191172 |
| 218120424 | rs2372946 |
| 218120624 | rs10168238 |
| 218120863 | rs7586158 |
| 218121113 | rs7600412 |
| 218121293 | rs10183545 |
| 218121786 | rs7589722 |
| 218122009 | rs6740850 |
| 218122107 | rs17778299 |
| 218122353 | rs17778329 |
| 218123052 | rs10198784 |
| 218123311 | rs4442975 |
| 218123663 | rs10932693 |
| 218124158 | rs11291245 |
| 218124274 | rs2888456 |
| 218124419 | rs2372956 |
| 218124426 | rs2888457 |
| 218125505 | rs11674902 |
| 218125555 | rs17778427 |
| 218126147 | rs17835044 |
| 218126358 | rs7588345 |
| 218126446 | rs7561690 |
| 218126695 | rs7561918 |
| 218126803 | rs7562029 |
| 218126936 | rs13000023 |
| 218127398 | rs17835187 |
| 218127601 | rs3085312 |
| 218127603 | rs3085315 |
| 218127665 | rs735361 |
| 218128212 | rs17835248 |
| 218128870 | rs13409592 |
| 218129396 | rs16856867 |
| 218129599 | rs2372957 |
| 218129616 | rs2372958 |
| 218129746 | rs5838621 |
| 218129748 | rs3085335 |
| 218129820 | rs16856873 |
| 218129963 | rs17778798 |
| 218130041 | rs16856877 |
| 218130142 | rs7600279 |
| 218130798 | rs11682933 |
| 218130803 | rs4608489 |
| 218131549 | rs13034362 |
| 218132117 | rs10211044 |
| 218133211 | rs16856888 |
| 218133717 | rs5838622 |
| 218133723 | rs5838623 |
| 218133948 | rs2287288 |
| 218134039 | rs2287289 |
| 218134273 | rs16856890 |
| 218134618 | rs7598618 |
| 218134620 | rs7598805 |
| 218134761 | rs7598926 |
| 218134805 | rs13018411 |
| 218134901 | rs7599066 |
| 218135359 | rs13415112 |
| 218135444 | rs6754177 |
| 218135688 | rs4361105 |
| 218135854 | rs13025492 |
| 218136008 | rs13025714 |
| 218137327 | rs12328709 |
| 218137594 | rs16825209 |
| 218137658 | rs12329133 |
| 218137683 | rs12329135 |
| 218137973 | rs6734010 |
| 218138557 | rs12622536 |
| 218138666 | rs13022815 |
| 218138690 | rs12996888 |
| 218138928 | rs12997141 |
| 218139101 | rs12328842 |
| 218139274 | rs12328926 |
| 218139288 | rs13002378 |
| 218139595 | rs16856893 |
| 218140109 | rs12478570 |
| 218140238 | rs12467063 |
| 218140453 | rs11690147 |
| 218140482 | rs13010040 |
| 218140537 | rs12467163 |
| 218140719 | rs13011060 |
| 218140839 | rs4672819 |
| 218140888 | rs4672820 |
| 218140952 | rs13011326 |
| 218140976 | rs11690464 |

TABLE 22
List of all dbSNP125 markers in the rs3803662 Linkage Disequilibrium
block (positions are from NCBI Build 34):

| Pos in | |
|---|---|
| Build 34 | Name |
| 52291041 | rs1076479 |
| 52292193 | rs7188075 |
| 52292669 | rs28720857 |
| 52293212 | rs11642868 |
| 52293398 | rs5816849 |
| 52293430 | rs11454608 |
| 52293504 | rs9937283 |
| 52294560 | rs12921126 |
| 52295118 | rs189851 |
| 52295192 | rs28558853 |
| 52295650 | rs7198218 |
| 52295682 | rs11337972 |
| 52295683 | rs5816850 |
| 52296050 | rs7203597 |
| 52296074 | rs11304815 |
| 52296100 | rs7203778 |
| 52296956 | rs4784218 |
| 52297566 | rs12444429 |
| 52297574 | rs12447254 |
| 52297595 | rs12447113 |
| 52297649 | rs28689711 |
| 52297698 | rs3095662 |
| 52297718 | rs28493012 |
| 52298081 | rs4493036 |
| 52298807 | rs7205259 |
| 52299817 | rs11464652 |
| 52299822 | rs10641032 |
| 52299825 | rs11427655 |
| 52299952 | rs1111480 |
| 52300224 | rs12447430 |
| 52301045 | rs43143 |
| 52301313 | rs187679 |
| 52302076 | rs42542 |
| 52302193 | rs4784219 |
| 52302954 | rs11649553 |
| 52303160 | rs3086679 |
| 52303163 | rs9923098 |
| 52303361 | rs8061425 |
| 52303886 | rs1978343 |
| 52304653 | rs3095595 |
| 52304699 | rs12102837 |
| 52305016 | rs10637120 |
| 52305075 | rs9931647 |
| 52305085 | rs28484983 |
| 52305086 | rs10637121 |
| 52305875 | rs7188610 |
| 52305996 | rs7186907 |
| 52306637 | rs9923960 |
| 52306726 | rs2193092 |
| 52306777 | rs2216188 |
| 52306900 | rs9939506 |
| 52307320 | rs9926539 |
| 52307440 | rs9302555 |
| 52308124 | rs12597838 |
| 52308541 | rs28562017 |
| 52309356 | rs8064219 |
| 52309429 | rs11287146 |
| 52309445 | rs8044985 |
| 52310401 | rs9928129 |
| 52310846 | rs3086687 |
| 52310948 | rs1111481 |
| 52311095 | rs2077653 |
| 52312052 | rs8061860 |
| 52312760 | rs8051542 |
| 52314189 | rs11427470 |
| 52314348 | rs16951213 |
| 52314403 | rs4784220 |
| 52314941 | rs8046985 |
| 52315230 | rs28665779 |
| 52315478 | rs12598982 |
| 52316033 | rs10451108 |
| 52316119 | rs4784221 |
| 52316179 | rs4784222 |
| 52316601 | rs7195542 |
| 52316633 | rs17271951 |
| 52316669 | rs12934513 |
| 52316689 | rs12934308 |
| 52316930 | rs7184835 |
| 52317345 | rs10545412 |
| 52317493 | rs12600239 |
| 52318190 | rs9933638 |
| 52318304 | rs28530752 |
| 52318333 | rs7186498 |
| 52318957 | rs13337151 |
| 52321003 | rs8063421 |
| 52321733 | rs11326759 |
| 52321900 | rs4541074 |
| 52321923 | rs4517793 |
| 52322238 | rs9302556 |
| 52322671 | rs7190749 |
| 52322726 | rs16951220 |
| 52323043 | rs1362544 |
| 52323132 | rs1819860 |
| 52323870 | rs7500427 |
| 52325826 | rs16951223 |
| 52326630 | rs12443621 |
| 52326776 | rs9923558 |
| 52326895 | rs9933556 |
| 52327297 | rs1362545 |
| 52327389 | rs4538010 |
| 52327503 | rs12922895 |
| 52327601 | rs12923628 |
| 52327690 | rs11415785 |
| 52327938 | rs7198754 |
| 52328239 | rs9936081 |
| 52328264 | rs8051286 |
| 52328472 | rs10647102 |
| 52328482 | rs5816851 |
| 52328483 | rs10645216 |
| 52328493 | rs3086690 |
| 52328528 | rs1362546 |
| 52328629 | rs2193093 |
| 52328900 | rs2160474 |
| 52328944 | rs12932707 |
| 52329434 | rs9939775 |
| 52329968 | rs8048043 |
| 52333201 | rs3086693 |
| 52333595 | rs1075367 |
| 52334335 | rs8046979 |
| 52334886 | rs1345388 |
| 52335310 | rs9941100 |
| 52337266 | rs1420529 |
| 52337322 | rs1420530 |
| 52337427 | rs1420531 |
| 52337486 | rs10708737 |
| 52337500 | rs11642645 |
| 52338806 | rs12918816 |
| 52340832 | rs4280232 |
| 52341080 | rs7197481 |
| 52341404 | rs12929984 |
| 52342090 | rs1420532 |
| 52342219 | rs1420533 |
| 52342476 | rs1362547 |
| 52342544 | rs1362548 |
| 52342856 | rs10538952 |
| 52343467 | rs28555277 |
| 52343743 | rs7193453 |
| 52344234 | rs9926163 |
| 52345047 | rs2193094 |
| 52345209 | rs28580623 |
| 52345455 | rs3095598 |
| 52346438 | rs12929605 |
| 52346440 | rs12929606 |
| 52347923 | rs28750243 |
| 52349175 | rs3112583 |
| 52349177 | rs3112582 |
| 52349414 | rs28435355 |
| 52349558 | rs11648477 |
| 52350025 | rs4783780 |
| 52350122 | rs3112581 |
| 52350194 | rs3112580 |
| 52350390 | rs11431881 |
| 52350614 | rs9921569 |
| 52351417 | rs9931232 |
| 52352109 | rs11075535 |
| 52353864 | rs12597887 |
| 52354487 | rs3112579 |
| 52354492 | rs4784223 |
| 52355066 | rs9933351 |
| 52355242 | rs1548910 |
| 52355727 | rs8052175 |
| 52355897 | rs28523905 |
| 52356089 | rs1420534 |
| 52356352 | rs7195388 |
| 52356420 | rs1123428 |
| 52356800 | rs1420535 |
| 52357133 | rs1477074 |
| 52358132 | rs3891563 |
| 52358441 | rs13336638 |
| 52358491 | rs8044585 |
| 52358571 | rs8045285 |
| 52358832 | rs4784224 |
| 52358870 | rs3095601 |
| 52359085 | rs3095602 |
| 52359653 | rs3095603 |
| 52360007 | rs12930156 |
| 52360562 | rs3095604 |
| 52360995 | rs9921056 |
| 52361357 | rs1362549 |
| 52361398 | rs1362550 |
| 52361636 | rs28463809 |
| 52361725 | rs4784226 |
| 52362126 | rs13335680 |
| 52362449 | rs3095605 |
| 52362775 | rs3095606 |
| 52362897 | rs3095607 |
| 52363302 | rs12926842 |
| 52364042 | rs3112578 |
| 52364943 | rs3803662 |
| 52365079 | rs3803661 |
| 52365170 | rs3803660 |
| 52365183 | rs3803659 |
| 52365510 | rs8053294 |
| 52365977 | rs12446056 |
| 52366465 | rs4322659 |
| 52366478 | rs28409238 |
| 52367399 | rs13335398 |
| 52367692 | rs7189773 |
| 52367823 | rs7191347 |
| 52367830 | rs13332085 |
| 52368150 | rs3104771 |
| 52369037 | rs28478483 |
| 52369057 | rs28418561 |
| 52369268 | rs8057406 |
| 52369377 | rs7199494 |
| 52369741 | rs28636082 |
| 52370476 | rs11437687 |
| 52370482 | rs3112577 |
| 52370737 | rs28719661 |
| 52370774 | rs28700577 |
| 52371185 | rs3104808 |
| 52371458 | rs28595571 |
| 52372064 | rs3104812 |
| 52372504 | rs16951246 |
| 52372582 | rs7201350 |
| 52372830 | rs3104818 |
| 52373327 | rs16951251 |
| 52373331 | rs3104827 |
| 52373794 | rs28715219 |
| 52373795 | rs28759054 |
| 52373894 | rs28491753 |
| 52374821 | rs28397852 |
| 52375020 | rs28561462 |
| 52375173 | rs28366779 |
| 52375215 | rs28690015 |
| 52375335 | rs28472996 |
| 52375668 | rs28437002 |
| 52375894 | rs3104743 |
| 52376140 | rs9922732 |
| 52376155 | rs3112576 |
| 52376446 | rs4594251 |
| 52376504 | rs28685217 |
| 52376699 | rs3112575 |
| 52377560 | rs3112574 |
| 52377659 | rs9937101 |
| 52377665 | rs16951253 |
| 52377790 | rs4784227 |
| 52377970 | rs16951254 |
| 52377996 | rs3104744 |
| 52378688 | rs3104745 |
| 52378902 | rs3112573 |
| 52378941 | rs13334992 |
| 52379032 | rs28503301 |
| 52379049 | rs3112572 |
| 52379220 | rs4784228 |
| 52379400 | rs12919508 |
| 52379702 | rs3104746 |
| 52379713 | rs3112571 |
| 52380052 | rs11372822 |
| 52380131 | rs11646414 |
| 52380217 | rs8053784 |
| 52380227 | rs11342984 |
| 52380243 | rs3112570 |
| 52380252 | rs9934281 |
| 52380383 | rs3112569 |
| 52380473 | rs3112568 |
| 52380526 | rs3104747 |
| 52380961 | rs3104748 |
| 52380977 | rs3104749 |
| 52381045 | rs3112567 |
| 52381091 | rs3104750 |
| 52381630 | rs3104751 |
| 52381735 | rs3104752 |
| 52381752 | rs3112566 |
| 52381753 | rs3112565 |
| 52381770 | rs3112564 |
| 52381945 | rs5011736 |
| 52382513 | rs3104753 |
| 52382735 | rs11864809 |
| 52382879 | rs16951272 |
| 52382932 | rs11859735 |
| 52383045 | rs16951279 |
| 52383698 | rs3481 |
| 52383902 | rs16951281 |
| 52384112 | rs3104754 |
| 52384265 | rs3112563 |
| 52384839 | rs3104755 |
| 52385009 | rs3104756 |
| 52386865 | rs3112562 |
| 52387168 | rs3104757 |
| 52387274 | rs10521267 |
| 52387635 | rs3104758 |
| 52387700 | rs9921358 |
| 52388131 | rs3104759 |
| 52388189 | rs3112561 |
| 52388416 | rs3104760 |
| 52388424 | rs3104761 |
| 52388458 | rs3112560 |
| 52388606 | rs3112559 |
| 52389436 | rs3104762 |
| 52389606 | rs8055502 |
| 52390581 | rs5816853 |
| 52390586 | rs5816854 |
| 52390646 | rs1109951 |
| 52390740 | rs8061255 |
| 52391109 | rs28636558 |
| 52391155 | rs5816855 |
| 52391221 | rs3112638 |
| 52391267 | rs9708611 |
| 52391639 | rs12935019 |
| 52392183 | rs3104763 |
| 52393309 | rs5816856 |
| 52394968 | rs5816857 |
| 52395219 | rs5816858 |
| 52395420 | rs11284113 |
| 52395423 | rs11326465 |
| 52395448 | rs6498958 |
| 52396694 | rs3112637 |
| 52396715 | rs4784230 |
| 52396799 | rs13330592 |
| 52397331 | rs5816859 |
| 52397555 | rs11645620 |
| 52398233 | rs3112636 |
| 52398720 | rs3112635 |
| 52398860 | rs3112634 |
| 52399117 | rs16951316 |
| 52399179 | rs3112633 |
| 52399652 | rs28567699 |
| 52399807 | rs3112632 |
| 52399850 | rs3112631 |
| 52399948 | rs5816861 |
| 52400091 | rs13335960 |
| 52400138 | rs3112630 |
| 52400341 | rs3104764 |
| 52400714 | rs11640537 |
| 52401021 | rs3112629 |
| 52401314 | rs3112628 |
| 52402239 | rs6498960 |
| 52402254 | rs5816863 |
| 52402438 | rs4262942 |
| 52402568 | rs3104765 |
| 52403048 | rs2335 |
| 52403100 | rs10653756 |
| 52403137 | rs3104766 |
| 52403194 | rs7201410 |
| 52403223 | rs12934093 |
| 52403340 | rs3104767 |
| 52403795 | rs28400414 |
| 52403802 | rs3112626 |
| 52404000 | rs7190208 |
| 52404011 | rs28379686 |
| 52404038 | rs17292403 |
| 52404154 | rs3112625 |
| 52404215 | rs12920540 |
| 52404998 | rs3104768 |
| 52405153 | rs3112624 |
| 52405181 | rs5816864 |
| 52405833 | rs3104769 |
| 52405970 | rs3104770 |
| 52406122 | rs11645834 |
| 52406128 | rs3112623 |
| 52406232 | rs8046543 |
| 52406857 | rs12930738 |
| 52406859 | rs12926392 |
| 52406869 | rs12926394 |
| 52406870 | rs12930747 |
| 52406881 | rs12102546 |
| 52406905 | rs28642024 |
| 52406977 | rs7198019 |
| 52407020 | rs11860998 |
| 52407095 | rs7192828 |
| 52407116 | rs7193515 |
| 52407139 | rs11860769 |
| 52407166 | rs11861036 |
| 52407928 | rs3104772 |
| 52408038 | rs3104773 |
| 52408105 | rs3112622 |
| 52408239 | rs10692294 |
| 52408304 | rs3104774 |
| 52408429 | rs3104775 |
| 52408504 | rs7200735 |
| 52408747 | rs7203671 |
| 52408951 | rs3112621 |
| 52409006 | rs7206289 |
| 52409053 | rs7206313 |
| 52409255 | rs7204612 |
| 52409498 | rs3112620 |
| 52409610 | rs3112619 |
| 52409628 | rs8062962 |
| 52409779 | rs12597100 |
| 52409788 | rs3112618 |
| 52410064 | rs3104776 |
| 52410319 | rs3112617 |
| 52410349 | rs3112616 |
| 52410566 | rs11075551 |
| 52410952 | rs3112615 |
| 52411233 | rs3104777 |
| 52411546 | rs12929797 |
| 52411635 | rs3112614 |
| 52412049 | rs7202142 |
| 52412254 | rs3104778 |
| 52412259 | rs3104779 |
| 52412298 | rs8044824 |
| 52412310 | rs10665605 |
| 52412516 | rs3104780 |
| 52412602 | rs3104781 |
| 52412765 | rs3112613 |
| 52412855 | rs8044760 |
| 52413602 | rs12922061 |
| 52413719 | rs28709052 |
| 52413766 | rs3112612 |
| 52413932 | rs9925692 |
| 52414779 | rs3104782 |
| 52414844 | rs3104783 |
| 52414967 | rs3104784 |
| 52415402 | rs3104785 |
| 52415965 | rs3104786 |
| 52416037 | rs4578643 |
| 52416145 | rs3104787 |
| 52416382 | rs12597685 |
| 52416795 | rs11374841 |
| 52417105 | rs3104788 |
| 52417150 | rs3104789 |
| 52417212 | rs12599893 |
| 52417265 | rs3104790 |
| 52417361 | rs4290467 |
| 52417477 | rs4467067 |
| 52417564 | rs3104791 |
| 52417836 | rs4238749 |
| 52417838 | rs4238750 |
| 52418217 | rs8048809 |
| 52418357 | rs4459541 |
| 52418357 | rs1112135 |
| 52419612 | rs3104792 |
| 52419788 | rs3104793 |
| 52420562 | rs3104794 |
| 52420628 | rs3104795 |
| 52420979 | rs3104796 |
| 52422526 | rs8046994 |
| 52422537 | rs7193357 |
| 52422543 | rs7186461 |
| 52423052 | rs3104797 |
| 52423497 | rs3104798 |
| 52424706 | rs3104799 |
| 52424978 | rs3104800 |
| 52425772 | rs3112611 |
| 52427035 | rs12921031 |
| 52427036 | rs12921032 |
| 52427156 | rs3112610 |
| 52427333 | rs3112609 |
| 52427444 | rs8045104 |
| 52427468 | rs3112608 |
| 52427539 | rs12921801 |
| 52427910 | rs6498966 |
| 52428293 | rs4488440 |
| 52428649 | rs3112607 |
| 52429238 | rs5816865 |
| 52429511 | rs3112606 |
| 52429906 | rs5002422 |
| 52429965 | rs3112605 |
| 52430198 | rs3112604 |
| 52430316 | rs11075561 |
| 52430713 | rs12599215 |
| 52431082 | rs4640177 |
| 52431277 | rs5816867 |
| 52431322 | rs4638564 |
| 52431382 | rs11647405 |
| 52431582 | rs11075562 |
| 52431715 | rs3112603 |
| 52431739 | rs4638563 |
| 52431878 | rs4426347 |
| 52431941 | rs3104801 |
| 52432313 | rs3112602 |
| 52432373 | rs11434012 |
| 52432387 | rs6498968 |
| 52432454 | rs3104802 |
| 52432459 | rs3104803 |
| 52432718 | rs7198792 |
| 52432830 | rs13338987 |
| 52433165 | rs11643841 |
| 52433198 | rs3104804 |
| 52433468 | rs3104805 |
| 52433888 | rs3112601 |
| 52433897 | rs12924424 |
| 52433898 | rs12924425 |
| 52434496 | rs28680336 |
| 52434497 | rs5816868 |
| 52434960 | rs7202932 |
| 52435190 | rs3112600 |
| 52435864 | rs3104806 |
| 52436127 | rs3104807 |

TABLE 23
HapMap SNP markers used for investigation
in the Multiethnic Cohort:

| | |
|---|---|
| European | Yoruba |
| Americans | (African) |

| SNP | r²* | D′ | r² | D′ |
|---|---|---|---|---|
| rs12922061 | 0.39 | 0.81 | 0.03 | 1 |
| rs4784227 | 0.81 | 1 | 0.03 | 1 |
| rs17271951 | 0.88 | 0.96 | 0.03 | 1 |

*r² and D′ values with respect to rs3803662

TABLE 24
Breast Cancer Association Data from the Multiethnic Cohort:

| | | | | | | |
|---|---|---|---|---|---|---|
| SNP | p-val | r | #aff | aff. freq | #con | con. freq |

European Americans

| | | | | | | |
|---|---|---|---|---|---|---|
| T-rs3803662 | 0.0214 | 1.23 | 534 | 0.3408 | 572 | 0.2951 |
| T-rs12922061 | 0.0724 | 1.19 | 534 | 0.2799 | 572 | 0.2462 |
| T-rs4784227 | 0.0036 | 1.32 | 534 | 0.3034 | 572 | 0.2480 |
| C-rs17271951 | 0.0154 | 1.26 | 534 | 0.3096 | 572 | 0.2630 |

Latina

| | | | | | | |
|---|---|---|---|---|---|---|
| T-rs3803662 | 0.0498 | 1.22 | 425 | 0.4190 | 426 | 0.3723 |
| T-rs12922061 | 0.2487 | 1.13 | 425 | 0.3231 | 426 | 0.2972 |
| T-rs4784227 | 0.1373 | 1.17 | 425 | 0.3429 | 426 | 0.3091 |
| C-rs17271951 | 0.4867 | 1.08 | 425 | 0.2905 | 426 | 0.2753 |

African Americans

| | | | | | | |
|---|---|---|---|---|---|---|
| T-rs3803662 | 0.0069 | 0.77 | 432 | 0.4747 | 457 | 0.5395 |
| T-rs12922061 | 0.0358 | 1.41 | 432 | 0.1065 | 457 | 0.0777 |
| T-rs4784227 | 0.0853 | 1.34 | 432 | 0.0972 | 457 | 0.0744 |
| C-rs17271951 | 0.2060 | 1.24 | 432 | 0.0922 | 457 | 0.0755 |

TABLE 25
Breast Cancer Risk Associated with rs13387042 allele A and
rs3803662 allele T in Estrogen Receptor and Progesterone
Receptor Positive and Negative Tumors.

| rs13387042 (A) | | | rs3803662 (T) | | |
|---|---|---|---|---|---|
| Cases | OR | P | Cases | OR | P |

Estrogen Receptor (ER)

| | | | | | | |
|---|---|---|---|---|---|---|
| ER pos | 2124 | 1.22 | 4.3E−09 | 2128 | 1.32 | 5.8E−13 |
| ER neg | 589 | 1.06 | 0.37 | 589 | 1.07 | 0.30 |

| | | | | |
|---|---|---|---|---|
| ER pos vs ER neg | 1.15 | 0.036 | 1.21 | 0.0098 |

Progesterone receptor (PR)

| | | | | | | |
|---|---|---|---|---|---|---|
| PR pos | 1802 | 1.23 | 2.8E−08 | 1805 | 1.30 | 9.3E−11 |
| PR neg | 816 | 1.12 | 0.036 | 817 | 1.16 | 0.015 |

| | | | | |
|---|---|---|---|---|
| PR pos vs PR neg | 1.09 | 0.150 | 1.13 | 0.057 |

TABLE 26
Flanking sequences for rs4848543 and correlated
HapMap SNP markers with r² > 0.2

>rs4848543
GCACTAGAAGGGTTACATACAATGGGAAACTGAGGCCCAGAAGGAGGAAA
GATGAAGTGCCCAAAGTTAAGGAGATAAAAGCAGAAGTGCAGGTAAGAGA
AGGAGTATTCATATTACTGCAGGAATCCAAATTCTACAAGAACATACGCT
TGTTCTTTAAAGGGAGCCCTCGCTTTAAGCATTATTTGCCGATCAATACC
[A/C]
ATTAATAATTACAGGACATGGGCAAATATTCTAGCAAAGGTCCTGAGGT
CTTCCTAGATGTGCCCAAAATAACAAGGACTCCCCCCGCAGGTGGCACCA
AAAGTCGGAGGATGTGTTGGAAGCTGACCTTCCCCACCTGTCCCTGCCTC
ACCCACAGTCACCCACCCCTCTAGGATGTAATGGTGAATCATTCAACACA
T
>rs11680207
GTGACACTAGGGACGTGGGGGGAATGCAAGTGGGTCCAGACCTGGGGGCC
ACCAGTGCAGGAGCAAGGGCAGGCCGGCCATCAGAGCTGGGCGGGGAGGG
GGGCGCTGAGCTTGGCGTGGGAGTTTCCCCAACGGACTGTGTCTTCCAGG
GACCCTCGCCCCTCATGCCCCTCTGCGTCCTGAAGGGAGGAGGCACCACC
[C/T]
CTCTGTGGCATCTGCTTTTTAGGGCTGACGATGGAACAAGCTCATGGAC
AGGAGTGAAGCCCCTGGCACATAGCAGGTGCCACAGAAGGGTGGTGAAGT
CTGCCCTAGATCAGAGATCATTCAGGGGGTCTGTTAACTTGGATGAAGAA
GAAATACACTCTTATCACTAACATCAGGAAGGTAAGCAAGGAACCACACA
T
>rs7583824
AAAGGAAGGGATTTCTGGGCCTCAGAAAACCACATGCCAACGCATGGGGG
TATTAAGGATAGGGGCTCTTGAACGCCCAGGCTGGTAAAATCTAGAACGG
CAGAGGGAGGAGGGGTAATTTTTCTGGTTGGTAGAAATAAAGTGGGGATG
AGGAAGAGCACGAAAGTCTGTCGGCAAACCTGAAAGTGGGAGCGAGCTT
[C/T]
GTCAAATCCAGGGTCAACCTGAGCTCTCATTAATTGGGAAACTGGAGAG
TCAGATCCTCTAAAGAAAGTGGAAATATTTGTTCTAAAGAACCCCTGCCA
GGTCCCCGTTTACGTGACTCATAAAGAGGCCTTCAGGCAGAAAGGATCCC
AGTGCACCAGTAACAGAAGGGCTCCAGGACCAGAGGGAATGGACAGGGAA
C
>rs4848541
TGGCCTATAGTCGAGTAGATAGAGGGAGGGCTGATAACTAAGTGACTACA
ACAGGACCTCCCTGTGTAAGGGTTCTAGCTTATTTCCACACTCTTCTGGC
ACAGACAGGTTGGCAGGGCAGCTGGTAGCAGGCACAGCAGATGGAATGTC
AGGAATGAAATCCACAAGACTCCAAGCCCCTCCCAGGGTGAGCTTGCCAC
[A/G]
GGCCACACCAGGAAGCTGAGCTCCTCACCCAAAGCCTGCCAGTGCCTAC
GTGGCAGTCATTCCCTTGTCAACAGACTTCCCACTGGCTTCTCTTGCTCC
ATTATTAAAAGCAAGAACCAAAAACTTCACTGAGGGGATAGAATCCCACT
AATAAGCATGTCCACGCTTTGGGTCATCTTAGGGGCACAGAAAATCTGAG
T
>rs1446128
GAGACACTTTTCTGCCTGCCCAAAGTCACATAGCTCATTAGAGGCCAGGT
TGGGCCTGGAGTCTCTGGCAGGAATAATAGCTGAACTTTCATTCATTCCC
TAACCCACCTGTGTTGTGAGGGCCCTGAGGCCAGCATGTTCATGGCTGCA
GCCTCCAACCAACTGAGCTAATTGACAAGAAATCTGCTCTGCCTGATGCT
[A/G]
TGGGGGATACTCAGATTTTCTGTGCCCCTAAGATGACCCAAAGCGTGGA
CATGCTTATTAGTGGGATTCTATCCCCTCAGTGAAGTTTTTGGTTCTTGC
TTTTAATAATGGAGCAAGAGAAGCCAGTGGGAAGTCTGTTGACAAGGGAA
TGACTGCCACGTAGGCACTGGCAGGCTTTGGGTGAGGAGCTCAGCTTCCT
G
>rs4849766
CCAGGCTCAAGAGAGAAAGCCAGACCCTCTCTCCTTCCACCTACCTGAGA
GAGGCCTGTCCTCAGGTGTCTGGCTCACAGATGGGGGACCCAACTGGGAG
TCACTCCCTTCTGGAAGCCCCAGGCCCTATAAACAGGTCCCCGGCCTCAC
TGCTGGGCCTCGGCCTGGAGACAGCGGCTCAGTGCAACACAACCACGCCA
[C/G]
AGCTGATCAGGGGAAACCCACGCAGCAGTTGAGGGGGCTGGGGCTTTGC
GGGTGCAGTGAGGGGACTGCTTCCCTTCAGTGGAGTTGCTGCCGCAGGTG
AGGGGAGGAAGATGAATCTGAAGGTTCTAACGTCAGCCCACAGGCTGAAG
CGAAGGCTGGCCTGAAGTGTTCCACCTGGGCCCCCACCTGAGCGGCTGAA
G
>rs6715243
TGGGCCCCCACCTGAGCGGCTGAAGAGTTCTGTTTCCTCAAGGCCCACAG
AGCTCCTCAGAGGGAAGCTTTAGTAGCTATTTCCTTAGGAAGGCAAGTAT
GTGTGCAACATTTCCTAATAAAATGCAAAGGCTGTTGGAGGCACTCCAGC
CCCCTCATTTAGCTCTGAGGTGTAATTAATACATAAATTAAGCTCCTGCA
[G/T]
ACTCACAAATGTCCAAGAAAAAAAATGAAAAATATAATTAGTTTGCCCA
TCTGACTCCATCTGTCAGCTCTGAGCTCAATGCTCAACGTAGCAAATCCT
GAGGGCTTTGGTCTCGCATTGGCTGGGCCCGGCGCTGCTCCTGCCCTGCT
CCAGGGAAGTGGGCCCTCCGCAGCAGCTGCCCAGAGCCCGGGCTCTGGAC
T
>rs895398
TTACCAGCCCTCTCTCTCCCTTCTCCCCTTTGCCTTCCACACACAGCCTC
TTGTCCAGCCACTCCCTCTTCCTGGAAGGGCCTTACTGACAGAGTGCTGG
GGAGCCCTGTCAGCTGGCCACAGGTCTTAGCAAGGGCCACCCCCTCCTTC
AGGCCTTCACCGTGTCTTGGAACCTCCCACTGCCACAGCCTCAGCGACAC
[C/T]
GTCCTTAGATCCAGGCAGGTGGGCTGTGACCCGGGACTGCACTTTGGAA
GGCCTTGCTATCCTCCTCTGGCCATCTCCTTCTCCAGGGCCCGAGGGATC
TGCAGAACCGGAGGAACATGCCAACCTGAAACCTGGGCTGTCTTTATCCG
GCTGTACTCCACCTGGGATTTTCTGCCCGGATGGCATCAAGATCGCTTCC
A
>rs11684731
GCCTAAGCCAGAGGAGGCAGGAGGGTTTAGGGCACCAGCCAAGAAGTGCT
TTCCAGATGCTGAGTGGGATCCCGAATAAGGGAAAGGGCATCTAACATAG
AATGAGGAGGAGATAACACAAGGGTAGAAATAACCAAGAAAGGGGATTTC
TCTCAGATGGCTTTAGAGGTGCTCCCAGCCATTGCTGGGCTCACATGGCT
[C/T]
ACATGTCATAATTTTTGCCAAGGCGGTTTCACTGCCACCACCACCCCGC
TCAGCCTCCAGTGGCTTGTCCACCCATGGCTGTGGCCTCTAACTGGGCAG
GATTTTATAGGCTAGGAAGAAAAGTAGGGAATGTTAAGGGAGACGGAAGT
CAAGTTCAACTTGGAACCATCCCAGAATGTCCAGCAGGAAAGGCCCATGG
A
>rs12464139
GACACCTCACCTTGACACGCAGCCACCTGCCAGTGTTCACAAATGTTGGT
TTTCAGCTTTATGCGAGGGTAGGATCCAACAAGCTCATTCTTTAATGAAA
AAGAATGAAATTGTAAAATATCAGATCTTATTACATGTCCTAAGGGTAAC
TTTTATCTCATAACACTTTTGCTTCAATCATGAGTGTATAATTTCTATTT
[G/T]
ATTTTGTGTCTATGGGGTTAGGTGTGCAATGTAGAAAAGCTTGAAAACC
ATTGCCTTATACCCATTTGAAAAATGAACCACCAAACAAAGGCTTAAAAA
CATGAATATTTGTCAACCAAAAGGAGAACTCCTGGGCCTGGAAGAAACAT
CACGATTCAAGTGCTGCAGCACGAAAGAAGTTGAGGCTGGATTTGTATTC
A
>rs6754664
TCCAACAAGCTCATTCTTTAATGAAAAAGAATGAAATTGTAAAATATCAG
ATCTTATTACATGTCCTAAGGGTAACTTTTATCTCATAACACTTTTGCTT
CAATCATGAGTGTATAATTTCTATTTGATTTTGTGTCTATGGGGTTAGGT
GTGCAATGTAGAAAAGCTTGAAAACCATTGCCTTATACCCATTTGAAAAA
[G/T]
GAACCACCAAACAAAGGCTTAAAAACATGAATATTTGTCAACCAAAAGG
AGAACTCCTGGGCCTGGAAGAAACATCACGATTCAAGTGCTGCAGCACGA
AAGAAGTTGAGGCTGGATTTGTATTCAGCTTGACTGTTTGTAAAGCACCA
TGCACCATGCAACTGCCTTCAGTTCCTTTTTTTTTTCTTTCTTTTTTTTT
T
>rs6754799
TGAGTGTATAATTTCTATTTGATTTTGTGTCTATGGGGTTAGGTGTGCAA
TGTAGAAAAGCTTGAAAACCATTGCCTTATACCCATTTGAAAAATGAACC
ACCAAACAAAGGCTTAAAAACATGAATATTTGTCAACCAAAAGGAGAACT
CCTGGGCCTGGAAGAAACATCACGATTCAAGTGCTGCAGCACGAAAGAAG
[C/T]
TGAGGCTGGATTTGTATTCAGCTTGACTGTTTGTAAAGCACCATGCACC
ATGCAACTGCCTTCAGTTCCTTTTTTTTTTCTTTCTTTTTTTTTTTTTTT
TTTTTTGAGATGGAGTTTCGTTCTTGTCGCCCAGGCTGGAGTGCAATGGC
ACAATCTCGGCTCACTGCAATCTCCGCCTCCCAGGTTCAAGAGATTCTGC
T
>rs6759589
GTGGGGGCAGCTCCTTGTTTTGTTCCTCACTGTCATTTTCCAAAAGGCAG
ACAAGCCCTGGCCGCCTTATGCCCAGGGCTGAGTCCACACCATGCTACAC
GAGCACCTGAGGGGAGGCTCCTCTCCAAAGTGCATTACCAGGGCCTGTGC
TAAATTTTTCAGCAGCCTCTTGTCTCACTAATGGTCCCGGGAAATAGACT
[A/G]
AGCCTCCATCCCCAACTAAGTGCTCAGGCCTCCCCCACCTCTGTCCTGC
CTGGGCAAGAAGCCCAGCGCCGCCCCACAGCCCAGCTCTCCATGCAGCCT
CAGCTCCCAGTGGCCCTCTGCCAAGGACTTGCGCCTCTGCAGTGGGATGC
AGTGCCTCATTTTATCCATAATTGTACGTGGTCCCTGTGTGATGTCCGGG
G
>rs1562256
GGCCAGACCACACTCTGTGGGGTGTTGGGAGTTGGGTTTTCTTGCTGCAA
GGAATATATGTTCTTTAAGGCAGTCATCTGCCTTAAAGATCCTAAGGTGA
GGAATTAGGGGAGCCTTATACTTGAAAGGGAGGTCCCCTTTGGGCCTGGG
GAGCATTGGTCTGAAGGCTCAGGTATGGAGGATGCAGGAATATCTGCTGG
[C/T]
GAGGATTGAGCTGGAACCTCCCTCCTTCCCACTCCCATCTTAGTCCCTC
CGGCCTTTAAAAAAATTTATTTTATTTTATTTTTATTTTTTAAGATAGAG
TTTCACTCTTGTCACCTAGGCTGGAGCGCAATGGCTCGATCTCGGCTCAC
TGCAATTTCCACCTCCTGGGTTCAAGTGATTCTCCTCCCTTAGCCTCCCA
A
>rs1446125
GCTGGTCTCGAGCTGCAGACCTCAGGTGATCTGCCTGCCTCGGCCTCCCT
AAGTGCTGGGATTACAGGTGAGAGCCACCGCGCCCAGCAGACCAGACCCT
TTCTAATACCTTCTGACCCCAGCAGTGGGGCCCAGGCCCACCTAACATGG
CAGCCTGGATGCACCTCTCTGTTCCTAGGCCTGGTGCCTGGCTCAGTAAA
[C/T]
GTTTTTTTTTTTAATGATTGAAAGCTCATCTTTATGGTACTTAAAGTCT
GTTTCAATTACTAAGGGTCTTTGTCGCTTTTTTCTTAGGGCAGACTTGTC
CAGGCCAGACCACACTCTGTGGGGTGTTGGGAGTTGGGTTTTCTTGCTGC
AAGGAATATATGTTCTTTAAGGCAGTCATCTGCCTTAAAGATCCTAAGGT
G
>rs1446124
GCTGGCTGGGATTACAGGTGCCCATCACTATGCCCAACTAATTTTTGTAT
TTTTAGTAGAGACGGGGTTTCACCATGTTGGCCAGGCTGGTCTCGAGCTG
CAGACCTCAGGTGATCTGCCTGCCTCGGCCTCCCTAAGTGCTGGGATTAC
AGGTGAGAGCCACCGCGCCCAGCAGACCAGACCCTTTCTAATACCTTCTG
[A/T]
CCCCAGCAGTGGGGCCCAGGCCCACCTAACATGGCAGCCTGGATGCACC
TCTCTGTTCCTAGGCCTGGTGCCTGGCTCAGTAAACGTTTTTTTTTTTAA
TGATTGAAAGCTCATCTTTATGGTACTTAAAGTCTGTTTCAATTACTAAG
GGTCTTTGTCGCTTTTTTCTTAGGGCAGACTTGTCCAGGCCAGACCACAC
T
>rs11677262
TGATACATTATTTATGGATCAGGTGGCACAGCATTGCAGGCAGCAGCAGG
GCAAAGACAGTGTGGAATCAGAGTCGGGCAGCGTTGGATGGGATTCCGGT
TCTGCCACCACTGGCTATGTGATGGAAATCTACCACATCTCTCCAAGCCT
TGCTGTCCTGTTTTATCAAATGAGGACATTGTACTGAATCACAAGGTCTG
[C/T]
ACAATGCCCGTGCATGTGCCTGGAATCTTCCCCTCTGGTGTCCCCTCAA
TGCCCATGGTGCTTCCTGCACAGGGTGTACCATAGTTGCTTGCTGTCTCC
TGCTGAAGCCTGCTGTGAAGAGGGACTGTGTGGAGGTAAGGCAGTGGGCC
TCTCCCTGTGAGGTCCTCAGGGGGCTGAAGGCAAAGTCAGAAGTAAAAAA
A
>rs7565771
TCTTCTTCAGTATTTCCCTTCCTCTCTGAACACCGTTTCTTTTCCTTTCA
GATTATCTGACAGCCTTTTAATGAACACCTACTAAGTGCCCAGCACTTTC
CTGGGAACTTAATTCTTACAACCTGTTAAGAGGTCTATACACTCATTTTA
CAGTGGAGGAAATGGAGACTTAGCGGAGATGTGAAATTTGAACCAGGTCC
[A/G]
TGTGGTTTCAAAGCCTGCGCATGTTCCTTTACCCTATGGAAGCCTCCAG
AAGCCAAGGTCTCATCTCCCCTCTTCCCTGGAGGCCCCAAACCTGGGTAG
CTGTGCCCCTAGACCTTTGAGGGAGGGTCTCGAGCCATTATCTCCCTTAC
CTTTTCTTGCAGTGACTTGGCTTCTCCCAGGCTGCCGTAACCAGCAAACG
C
>rs838066
TCACCTTTCGTGGTCGTGGCACGTCCTCTGCTGGATTTTTGAGCCAAGGA
CAGAGGGTTTGGGAGAAGGAGGCCTCCTTGGACTTCTGCCAGGAGTTACT
GAGAAAAGAAAACCCAACCAGCTGGGGGGCCAGTTGGGGGGAAATCTGCA
CAGGATGTTCACACTGTGCCCCTCCCTGCTTCTTTCCTGACCCCCAGAGC
[C/T]
CACCTCAACACATCTGGAGTGTAGGGTGTTGTCTGCCCTGTCAGACCAC
TCTCCACACACAGCCTGGGGCAGGAACAATGGGACAGGTGGCTGGTGCTC
ACTCAGCACAGCAGGGGTGGTGGAAAGACACATGTCAGCTGAGGCTTGGC
TTCAGGGCAGGCAAGTCACAGCCAAGTCACAGCCCAGCCCAGCATAGGCA
A
>rs895397
TCAACGGTCTTGGCTTGTGACTAGACCAAGATCGCCAACCAGAATGGAGG
GAGGCTGACCCAGCACATCCCGGAAGCATTTACAGAGCCAAGGAAGAGAT
TCTTCTTGCCCGTCTATGATAACATTGCATTGCCTTTCAGCTGGGGGCGA
TTAGAAAGGCACTCACTACTAGATCATGGGAGATGCCTGGGTTTTAACCC
[C/T]
TTGCTGCTCGGAAGGTGGTGATCATGATGGCCTGGGAGGGTTTTAGAAT
GGCAGAGTGTCAGGCCACCCCAAAGCTCCCAAATTAGAATGGCATTTTAA
CAAGACCCCCTAGGAGGTTGTATACGTGTTTAAGTTTGAGAAGCGCTGGT
CTACATTACAACTGAGGAAACTCACTACTCCCTGAACACCTGCATCAAGG
T
>rs838059
ATAAGGAGCTTCAGGTTATTTTTAGGAAGTCTAAGAATCCTGTTTTCTGG
GGAGCCAGCCTCTGTTGGGGAGATAAGCCTCCGGAGGCCAGAGGCAGGAG
CTGGGCCTCAAGGAAGGCCCCACAACTGGACCCCAAAGGGCACGGTGGGG
AGGAAGAGGCAGAGCTCTGGTCTCCCTGTGGAGGGAGTGCTGCTGTAAAG
[C/G]
GCCTTTCCCTCCACCCCCAACTCCCGCCGAGAACCCCCCCACCCCCGCA
GGCAGTTTGCTTTCCCCCTCCACAGCTTTCCTCCTACCCGAATACCCACC
TCCCAGCTCTCTGCTCAGCCAGGGCCCAAGGCCAGAGCCTCTCTTCTCTT
GCAAGAACTTTCCTGTCCCACCTTGCCCCAGGGCAACACCTCTTATGGCA
A
>rs838102
CTAACTAACTGCCACATTCTCCTGTCCAAATTCTGCCTCATCGGTTATTC
ATTCAACAAACACTTACCGAGCACTAACTTTGAGCCAGGCACTCTAAGAC
CAGCTGAGTCCCACTTCTGGCAGGATGAGTGTTGTTGTCATCAACCACCC
CACAAACACCCGTGCCCTCTGCGTACTTGCGAATCTGAGTGAGGAGACAC
[A/G]
GTGGCAGTAAGGCTCAGGACCTATAGCTCATGTGAGTGAGCAAGGATGC
AAAACCAAAGACACCCGCTCCCCTCACCCACTGAGCTGGAGGGATCAACC
ACTGTGCCTGCCATCAGGGCTGATGGGCGACTGCTTGTGGATCACAAACT
CTTGCGTTGTCACTTCAACCCAGGGGCAGAGTGTGTTCCTCTTCAGGTGT
G
>rs3769659
GGGCCCAGCACTGTCTTGCTTCCTTCCCTTCCAGTTCCAGGCACCACCTT
CTTTCTGGCCAGTCCAGAGACATCTCCAAGGGCCAGAGGGTCATCTCCAG
AGGGCCTGGTGTCACTTCCCGCAGCTGCTCTGGCCTTTCACCCAGGTTGG
GGCGATTTGATGGACAGTGGTGGGTCCCCTCAAACTCCACTGCATTCAGC
[G/T]
TGCTACTGACTCATGTGCAGGCCTGGGCAGTCACCCAGAAAGCCCCCCT
GGATGCCAGCAAGCCCACTGTGGCAATGGGCAGGCCAGACCTCACCTGGA
GGGCCCAGCTCACAGCCACTGTATCCTCTAGCTGTGCACCCAGAGTTCAC
CTTCAGAGAGACACTTGCCCTGACTGGGGGTATACTGTTACTCTCTCAAG
C
>rs865108
AGCAGCCAGAAATCAGGGTGATGGGTGGGTGGAGGGGCAGGCATCTGGGA
AAGCCCTGCCCTCCTGGTGTTCCCAGATCTGAAGGCTGGAGGCTGGAAAC
CTTGTGTCACCTGTGCTGGTGTGAGTTCCATTGGGGCTGCCGCTTACCCT
CCCATGACCTTTAACATCTGGCATGGCTTGTGGGGGGTGCTGAGAGGAGG
[A/G]
GAAGTCTCAAGGCTGAGGGAGCTCAGGGTAGAGCCAAGGTCTCAGTGAG
AACCCCAGGGGGCAGCTGTCACAGCTGACCTGGAGCCCTCCAGGAACGCG
TACTCTCCAGCTCCTGTACTCTCAAAAGCAACAGAAAGGTTTGCTTTCCT
CAGGTGCGGTTTCCTCACAGTGCAGCTTCTCCAAAGAGATCTGGAGACCC
C
>rs708670
GCAGGGACTGACTTCCACTACCCCTCCTTCCAGTCCCTGCCCACTCCACC
CCCCACCACAGCCAATGGCATTCTTTCCATCTCCAATCTTGACAACCTCC
TGGCCCATCCCAATCCCCCTTCCTTAAAGATCCAGCCCAAGGCCTTGGGC
ACTTAAGGCACCTGCCATCTCCCCCAACGCCCACCCCTGACAATCTCTCC
[C/T]
GGCTTTGAATTCAGACAGGCAGCTGTGTTAGTTGGGGGACCTAGCTGGA
CAGAGATGTGTGTGAATTCCGGTCCAGCCATGTGCTAGCTGTGTGACGTT
GGGTAGGTTGCTTTCCCTCTCTGTCCTGATGATTAAACAGTGAATATGAA
ACCCCTAAACACATGTCTTCCTTAGGACTGTTCTTGTGTGCACCTTTCCT
T
>rs708672
CCTCATTGTTCTCTGACTGCTGTGGCCCCCTGGGTCTCCTCTCTTCTACT
GGAGTGAAAGCCCCTGTGGGCAGGGCTAGATTCCTGAATCTCCGCGGAGT
CTAACACAGGCCTGTAGGCAGGGACTGACTTCCACTACCCCTCCTTCCAG
TCCCTGCCCACTCCACCCCCCACCACAGCCAATGGCATTCTTTCCATCTC
[A/C]
AATCTTGACAACCTCCTGGCCCATCCCAATCCCCCTTCCTTAAAGATCC
AGCCCAAGGCCTTGGGCACTTAAGGCACCTGCCATCTCCCCCAACGCCCA
CCCCTGACAATCTCTCCCGGCTTTGAATTCAGACAGGCAGCTGTGTTAGT
TGGGGGACCTAGCTGGACAGAGATGTGTGTGAATTCCGGTCCAGCCATGT
G
>rs708673
AAACAGATGACAGCACTTGTCTCCAGGGCAAAGCATGGACGGGGGCTGAA
GAAGACACAGGCATGTTTCTGCTGGCTGGGGATGGCTTTTCAGTGGACCT
TGGAGGCCACACCGGCAGCCCAAAGGAGGCTGGAGGTTTGGAAGCTTTTG
CATTGTTGAAGTGAATTATGTGCAAGCCCATTCTTAGGGATAAGTGTATT
[C/G]
TATTGTTTTCAACTTGTTTGGAACCTAGCGATCTTGGAATAGGGCACAT
CTAAGTATCCCGGGCATCTTTATCTCAAGCCCACATGCAGAACCTGTTTT
TCAGATCCCTGAGCATCTGATAAACAGGTTCTGGGCAGGTTTCCCCTATC
ATCACCAAACACCAAGCCAAGGCCTCATTGTTCTCTGACTGCTGTGGCCC
C
>rs838100
ATCAGTCACCACTCCCGGTCCAGCCCCTGTGGCCAAGAGCTGGCGTGCAG
GCTGCGGGAGGCAGCTGGCTGTGCAAGACCCTGGCAGGGCCCTCGCCTCC
TGAGAAACCGAGAGTCAGAACCAAAGCCAGGCTGTCCTGGTTGGAGACTG
AGCCAGAAAGGGTGGCTCACCTCACGGTGAGGCTGTCGAGTGACCTGAGA
[A/G]
CCTCAGACCCTCACGTCAGCCGGATGTCGCACCAGCCTGCTGTTGGTAA
GTCTGGCTAGGACGCAGATCCAAGGGGGCATGGGTCGTGTGCAAACCCCT
CCCACCAGCTCCCTCACCACCTGACCCCTGCCCTGCTGGAGGGTGCCCCT
TGAATCTCCAGGAGGTCCGAGGAACTCGACACCTCCCAGAGATCTGTACT
G
>rs838098
AAATTTCAGGGGTTCAAGGATTTATCACACACAGTTGCGTGTGTGTGTGT
GTGTGTGTGTGTGTGTGTGTGTGTGTGTGTTTAAGGATGTTAATGTTTAT
GATGCATTTCTGTGGAATGCAGGCCCATTGGGAATTGTAAATTTTTCTTA
TTGGTAACCATTTCTGCATTGGTTTAGCACGGAACTGCTGCTGCAAGTAA
[A/T]
TGAACCAGATTTTTCTGGAATGATTCGAAAATCTAGCTGTTTGTAAAAA
GCTCTCTAAATTATTCATTCACTTTTATTTTGCACAGTTGTGTGACGGTA
AACTCTTTTCCATCAGACGAAACTTGCGCGCACGTGTATTTCTGCGGGGA
TTGGCTCGAGTGTGCTTGAGTTTTCTGTATTCTCTTTTGTAGGAACTTCT
C
>rs838096
ATGCCCACAGAGAAAAATAGAAATCTACCAATTAGATGTGTGTGGCTATA
AGTGAGCTTTTAAACCATGATTTGAGACTGGCATCCTCTGGCCTCTCTGC
CCTCAGGTCAGCTGTGGGAGGCTAGGGGTGGGATAGCAAAAGGGACCTGG
GATCAGGTGGGGCTTCCTGGACCCCAGGCCCAGTAAGAATGAAGCCTAGG
[A/G]
GTTCTAGGATAGGGGGCAGCCCTGGATTTGCAGAAACAGCCTGAGAATG
AGGGGCTGGAAAGTATGAGTTGGAGCAGCTCAGTGAGGAGACAGAAAACT
GAAGCAGAAGAAAGCAGAAGACACCAGAGTCTGGTGCTGACTGGGTGACT
GGCTGTTACTCACATCCTGAGCAATGAGCCACAGACCCTCCCCACCCCAG
A
>rs838092
CATTTCCCTGAGGGCTAATGATGCTGAACATCTTTTCATGTGCATTTGTA
TATCTTTTTTGGAGAAGTGTCTTTTCCAGTCTTTTGCCCACTTTATGTAG
GTTGTTTGTCTTTTTAATACCGAGGTGTAAGAGTTCTTCAGATATTTAGA
AACAGGTCCCCTTATCAGTTATATGATTTCCTGAACTTTTCTCCATTCTG
[A/T]
GTTGGCTTTGCACTTTCTCGATGCTGTGCTCTTTCGATTTCGTCAGCAG
CGAATTAGCAGGACCTGGGGGGCTTTAAGAGGTGAGCAGGCAGACAGACA
GCGCCCACAAAGTCCTGAGGATGAGAGAAGAGGCAGAGCTTACACAAAGG
CTCTGTGAAAACTGAAGATGGACCCCAGGCAGGTTGGGGTGATCCCTGGG
G
>rs838090
TCAGCCTCCGGAGTAGCTGGGATTACAGGCACGTGCCAACACACCCAGGT
AATTTTTGTATTTTTAGTAGAGATGGGGTATCACCATGTTGGCCAGGCTG
TGTGCAAGAATTTATGTTTTCACTTGTGGCTTGCTGAGCCCTGGTGCTGG
CTGTGGGCACATACTCGTTCACCCAGGGACATGCACTGTTGATGGGTCCT
[C/T]
GCACTCTGGGCTATCTTGGGCCCCGTTTGCTCAATGCAGGCCCCTCTTT
CTGAAGCGCTTTTCTTGGGCTCACTCTCAATTCGTCAAAGTTCGGCTCAG
GCCCACCTCCTGCAGGAATCCTCCCAGTCCCCGCTGTCCGAAGTCAGTAC
CCTTGTCAGGAGACCTTAAACACTTTAGTCCACTGCCTGGCCCTCCCATG
G
>rs12467944
AAGAGGCCGCCTAGGGAGCAGTACAAGGCAGCATTACAACCTGGCATTTT
CCTGTTGCAGACTGCATTTTGGTGGCAAGAGAAGCTTTGGAGGTGCTTAA
CGTTTAGTTTGCATTTATCCTACCGCTGAAAAGCAGGGACCCTTAGGACC
ATAAGTCTAGGACTTTTCATAGCACCCTAGACTGCTGGGGATGAAAGGGG
[C/T]
TTGGAGCTGAGCTGGTGCAGCAGCTCCTGTTAGAGAAGCCACCCAGAGA
GGCCCAGGGACCTCCTATGTCCCAGCGCCAGTTAGGGGCAGACGTGGGAC
CAGCTCCCCTCGGCTGCAGCGGCCTCCATATCAGCTCCCTGGAAGGGAGC
AGATGCCAAGTCAGGTTCTGAGCAACACCCTCAAGGCCCAGCCCTGGCAA
T
>rs10196144
GACTTGGCCCTCCCGACCACAGGAGAAGAGGGATTTGAATTCACCTCGGC
CCAAATTGCAGATGCTGGCCTGCGAGCCCTGAGGCCATCTGCAAGGCTCC
TGGGCTGACATAACTGTCACCCAGCTCTTCATCCTCCTTCCCCGGGGCTG
AAGGGGTGCCTGCAGGGTCTTCTGCCCTCCCTCCACCTGGGCCCTACTGC
[C/T]
GGCACGGAGCTTCATTTCTGTTGGCAGCCAGTGTTTGCTTAGTGATTAC
TGAATGCCGTTTGCTGGGTGCTTATGATGTGCTCAGCACTGTTCTAAGTG
CTTTACACAATTGACTTATTTACTCCTACAACCTGCCCATAAGGGAAGTG
CTGCCATGGTCTCCCTTTTATAGCTGAGAAAACTGAGGCACAGATAGGTC
C
>rs6728660
AGGCGGACTGGCTCCAGAGCCCGGCTCCTGGACCGAGCAGCCTTGAGTAG
GGACCTGGTGCAGACCCGCCTGCCGTGGACTCACTATAGGATGGGTCAGG
AGGCCCGTTGTCACCTACAAAATGTAGCCCACCACCCAGGAAACGCTCCC
AGAACCAGGCAGAAAATACTGTCTCCTATTCACAGGGTGGCTGTGCCTGG
[C/T]
GTGAGTGTCTTGGCGGGTGCCTGCATGTCTGTGCAGGAGTGCCACCCCC
ATCTGTGGACTCGGAAAGCATGATCCCCACAAAAGCAACCCCTCAGAGTC
TCAGCTCAGCCAATCCCTGTTATCCTGTGACAGGCAGGGCCATGTTTCAA
AACCATGTTAAAGATGAGCAAGAGGCTAAGGAAGTTTCCCATGCTGGCAG
A
>rs10188946
ATAATACCCATCGTAACAATAAATATAAATGGCTAATTTTCATCGAGTGC
TGCCAGCATGCTCTTTTTGATATGTTATCATAACTAATCCTGCATCACTG
CAAAATTCCATTTTACAGAACAGGAAATTGTGGCTCAAAGACATTAATGA
TCTTACTGAAAATTCCATAGCTGGTAAGTTGGGATTTGAACCCAGTTCCT
[A/G]
TGGATCTGAAACCCATGTTCTTTCTGCTATGGTAACAGGGTTTCCTAGA
GGGCAAGGAGGAAAAGGTTGATGGCTTTTTGCTATTTGGAGCCAAGAAGG
AGAGAGACTTGGCTTTTAGAATTGGACTCAGAAGGAGGAAAGTAACACAC
TTATGTACCCGGCCCTGAGGAAGGCAGGCATGTCTCAAATTTTATCGTAT
T
>rs895406
AGGAGGAATAGGAGTTTGCAAGCTGTCAAGGTGTCCTGGGGTGGGCGTTC
CAAGCCGAGAGGAATGAGCTCTGCCCATTTAGAGGACAGTATGGGACCCA
AGTGTTGACACGTCACAGGAGAAGGGGAGATGAGGCTGCGGAGGGAGTCT
AGCTGCATAGTGAGGGTTCTACCATTATCCCAAGGACACTGGGGTCACTG
[A/G]
ATCTTTTCAGCAGTGACATGGTCAGATGAGAGTTTCAGACCATCCTGTG
CCAGCCATGTGCAGATGTAGCTTGGGGTGAAGGTAATAGGAGAACGGGGT
GAAGGTAACAGGAGAACAGTGTGCAGGTTAAACATTTCTTTGATCTTGAT
CTTTGAGTTTGATGTTGAAATGTGTATTTCCAGGGAAATACACAAGCAAA
T
>rs708675
TGGACCTCTGAGTCGGGTATGAAGTAGTGTGTAAGAAAAATAATAAAAAA
GTATTTACAAAATGCAACAGATTACAGAGGCAGGTATGGGGAAGTAAGGG
GATAGTCTGGGGGAGCTTAACAGAGGTAGGGTTTTGAAGGAGGAATAGGA
GTTTGCAAGCTGTCAAGGTGTCCTGGGGTGGGCGTTCCAAGCCGAGAGGA
[A/G]
TGAGCTCTGCCCATTTAGAGGACAGTATGGGACCCAAGTGTTGACACGT
CACAGGAGAAGGGGAGATGAGGCTGCGGAGGGAGTCTAGCTGCATAGTGA
GGGTTCTACCATTATCCCAAGGACACTGGGGTCACTGGATCTTTTCAGCA
GTGACATGGTCAGATGAGAGTTTCAGACCATCCTGTGCCAGCCATGTGCA
G
>rs1867856
CCATACTGTCCTCTAAATGGGCAGAGCTCATTCCTCTCGGCTTGGAACGC
CCACCCCAGGACACCTTGACAGCTTGCAAACTCCTATTCCTCCTTCAAAA
CCCTACCTCTGTTAAGCTCCCCCAGACTATCCCCTTACTTCCCCATACCT
GCCTCTGTAATCTGTTGCATTTTGTAAATACTTTTTTATTATTTTTCTTA
[C/T]
ACACTACTTCATACCCGACTCAGAGGTCCATGACTGGGTCTTCTACACC
TTGGGGTCCATGGTTCCTAACACAGGTTCACAGTGCATGCTCACTGAGTG
ACTGATTTTTAAATTGGCCACCACAGAGCCAATATCAGGCATCTTAAGAG
GAGTGCACGTAGAGCTTGGAGCAAGAACTAGTCTAACTGCCTTGCTGCCA
C
>rs12711924
CCTGATCCCAGCTCTGCATCTGTTAGCTACATGTGACCTTGGGAAAGTGA
TTTGATCTTTGTGCCTCATTTCCTCCATCTCTAAAATGGGCTAATAGTGA
TCTCTACTTTGTGGGGTGGTTTTGAGGATTCGATGAGTTAATGCCTGAGA
AACGCTTATCACAGAGCCAGGTAGCGTCAGGTGCAGTGTGAGCGCCCGCC
[A/G]
TCTTGTCTTTCTTGTGTCCTTCATCATTGCCCTCCCGGGGCACCAGCCT
TTGCCACAGTGTTCATGGTTTTCCTTCCATCCATGTCAGGTCTTGGCCAA
CAAGAGCCACCTCTGGGTGGAGGAGGAGGTCTGGCGGATGGAGATCTACC
TCTCCCTGGGAGTGCTGGCCCTCGGCACGTTGTCCCTGCTGGCCGTGACC
T
>rs1867749
GGACTCAGGGACAGTGACAGCGTAATGCTCTTACGTAATCCAAATTAATT
GCTCCTGACCAGGGATGCTTCACATCCATTGGAACAACACAGAGTTGTAG
CCGTGTGTTTTTTAAAGTGTGTCTGCCTCTGAATCTCCCTGGCTCTGCTC
ATCTCAGAAGGTCCCTGGAGGAGGGCTCATGGCTGACACATGTTAGCATA
[C/G]
GAGAAGGGGAAGAATTTTATTTATCTGCCACTAATCCCCATGTGTCTCA
TACCCAAATCTAGGATTTTCCTCTTCCCACCTGGAGTATCCGGCGTGCAT
GGTTTATCCACAATTAAAGCAGACTGCAGGCTGCTCCCCTCACCAGAAAG
CCGCTCCCTTAAGCAGCCATAGATTACATGCCCTGGCTCTGCCTGCCAGA
G
>rs838073
TGCTCCAAGAGCCTCAGTTTACACCCCTGAAAAATGGAGTTAATGCCTTA
CAGGGATTTTATGAGCCACAGTAAGAGATTATGTGTGAAAGAAAAATAAA
AAGCATTTTGCTATTATTCTTAGGGTATCTGGCCACATTTCCATTATCTC
TGACTAAGGTACCAACCACCGATTTCTGAGTTCAGTAGGAGACAGTGAGA
[C/T]
AGAGGCCAAGGAACCCCAACCCTGGGCCTAAAAGTGGTCCAGTCAGTGT
TTGCAAACTGGCGGACACTCACTAGTGGGTCATGCAATTGATTCCATGGA
TCACAAGCAGCATTTTAAAAATGAAATAAAATAGAATGACAAAGAAAACA
GTGGAGTGTTTTGCATGGTGTCGGGATACTTTTTCAGTTATATGGAATTG
C
>rs838069
CTGACCTCATGATCCGCCTGCCTCGGCCTTCCAAAGTGCTGGGATTATAG
GCGTGAGCCACCGCACCCAGCCTTCCTTTGGTATTTTTTTGTTATTGTTT
TGGTTTGGTTTGGGTTGTGCTAAGTGTTTCAATTCAGCCAGTTTTAACTG
GCTGTTAGATATTAGACTGCAAAATACTCCAGTCAGAGCAGCTCACCTGC
[A/G]
CAGAAAAATGAGGGTTCAGCTTTTAGAACCTAAGTTCTTTCCCAGAGTT
AGAGGAACTGCCTGTGACCTTGCCCACTCCAGCCACCTGCCTGAAATAAT
GACAAGACCAGCCTGACCCCAGCTTCATGAGCAATTGTCCAAAGCCCCTT
TGTGCCGTAGTAATTGAGCACCAGCAGGAGCTACAACGACCTGGCTGTTT
C
>rs3731603
TCCTGTGGCCCCGGCGGTGCTCTTCCCTCTTGTGCTTTGGGGCAGCAGCA
TTTTAGGTTTGTAGCTTTGGTGGAAAAGTTGCTCCCCACTGTTTTGAAGT
GTAATGCCCCATGAGGAGGAAGTTGGAGCCAGTGTGGTTTTCTGGGTCCT
CTGCCAGAGGACATTTGGAGTCCCCAATATTTTTCCTCAGGACTGACTTC
[G/T]
TGAAGACAGTCCTTCAGCCCAGGCAGGGCTCAACTCTGACGCCCGGGGA
GCTTTACTCCCTAGAGTGTTTTCTCTTGCTCTCCCCCTTGGGCTGATTTC
TAAAGGGTCTGCTCTAGGCCTTCCTGGGAGTCATTCAAACTGAAGATGGA
AAGGGTGAGAAGGCACAGTGGTTTAAAGGACGCTCATCGCCAGGTTAAAG
G

TABLE 27
Flanking sequences for rs13387042 and correlated
HapMap SNP markers with r² > 0.2

>rs13387042
CTGGCATCCGAAGGTATAGGCTCAAATCCCAGCTCTCATGATTGCTAGCT
TTGAGGTTTCAGGTTCATTTCTTAAGTGCTCTGAGCTTTGGTGCCCTCCT
CTTCAAAATGGGAATGAGGAGCATGTCTAGCTCCCTGTTTTGTTGCAGTG
AATAGCTACATGAAGAACAGCTAAACCAGAACAGAAAGAAGGCAAATGGA
[A/G]
GCTACAGAAACCAAGGATTTCCTTGTTGAATCGAATCTTCCTTCAATCT
TCCTTCACCACACTAGTGGATCTCCCTGTGGGAGGGATGTTGAGAGTGCT
CCGTGTTTTTTTTGTTTTTTTTTTTTTTTTTTTTGTGATGGAGTCTCACT
CTGTCACCCAGACTGGAATGCAGTGGTGCGATCTCGGCTCACTGCAACCT
C
>rs1882419
AGCCATGGGGGTGACACAAACTCCAACAAGCACCAGGGTCAAGTAGAAGC
TAACTGACCCAGGAGAGAAAATAATTCTGTCAAGTTAAGTAATTTGCTGG
TGTTCTCTGCTAACTCACATCTGCGTCCTGGGTCTCCTTAGCTCTTCCTT
GAAAAAGCAATGAATGTTTTCTAACTGTAGCTACCCCTAGTTCTTCAGCA
[C/T]
GTCTGTGGCTGGCTGCTTGCCACCCAGGCATGAGAGGATGCTGTCCTCT
GGGAGTCCCAGCCTTGTGGTAAAGAATGCAGGCTGAGAAAGACTGTAAGA
TTCATATTCAGGATCTTCCACTTGGTGGGCAACCCTAAGAAACTCATTTA
ATCTTTATTAGCCTCCGTTTCCCCTTCTGTAATAGTACCTGCCTCACAGG
G
>rs2252488
AGACATGGTCAGGGCCTCAGACCCTGTGTGTGGCCATTCAGCTTTGCCCT
GTTCTGTGGCCACAGTCCATGCTCTAAGTACTGACCAGATGTCTTACAAT
GCACACGGCACACAGTTATGGTCTCTGTGGGGATGGGGGAATAAGGTGTG
GACAAATCTGTTCAGCTCTAAATCCTTGACCAGAGGAGCAGCTCAGAGCA
[C/T]
GAATCCACAGGGCAGTGGAGCCATGTGCAGGATCAAGCACAGCGGCATC
ACTGACTTGCAGGACACTGGAGTGCTGGGGCGTGGTTCTAGAGAAATGCC
CTCAAGTCCTCGATAGATCCTAAATGTGGTTAGTTCTCTTCATTACAAAG
ACAGTGGAAGCTGAGGTCCATGCAGATTAAAAGGCTAAGCTGTACAGTAA
T
>rs2272525
GGACCCTGGTCAGCCTTAACACAGGCTGTGATCATCTCAAACCTTTCGAG
ACCTTTATCCATAAGAAAATAAGAGGAAATTTTATTTCAAAGTAGCATGA
AGTGATATGCCAACTCACTTGAACAGCATGACTATTTTCTGGTTCTGTTT
TGTAGTTACTTTTCACGTGGAGGCCCCATCTGCAAAATAGGACAAAGGAA
[A/G]
ACTCACTGAGACCAAGTCTGGGATTCTGAACCCCAGACCACTTTCTTCC
TCCTTCCCTCAAACACACTCACTCTCTCTCACTCTCTTCTCTTTCTCTCT
CTCTTTTTTCTCTTTCTTTCTCTCTCTCTCTCTCAGGTCCTGGGGCACTC
TGGACACAGTCTGAAAACCATTGAGGTACAGCAAGTACCACAGAATGGAA
T
>rs4396687
TTTCCAAGGACTTAAGCAAACAGAGTCAACTCCTCTGTTTTCCTTGTTTG
GGTATATTTTTGAAAATGTCTCTTCTTCTTTCCTTTCATTTACATTTTAC
CGTTGTTTATCCACCTTTTCCTCTTCCTTCAAAGCTCTAGACCTGCCTCC
CTGCCTGTTCTTAGCCCACTTTTGAGAAAGGACTTTCCAACCCCATCCTC
[C/G]
AAAGGGCAGGGTCTCTCTCCAGGGCTCCAACAGAGTTCTGAAAGAACAC
GGCTTCCACCATTGTGTCTTCTCTGTTTTGTCAACTGCTGAAAGACTGAC
ACAGTAGACAATGCTGCAGACCCCTTGGGAGATAGTCCAAGTTTAGGGTG
GGGAGGGAATGGTGCATTCTGGGTGTCCACATATTTGTTGTTGCTTTAGG
A
>rs12614767
ACAAGAAAAGGGAGTGAAGTGTGTGGGGTTTTTAAAGTGGTGGTGGGGGG
GCATTAGGAGATACCCAGAAGCTCTCAAGATTCCAAACAGTCTGCTTTTC
TTTTCAGACATTACACTCCAAGAAAAGAAACTCTAGTGTATAACAGAGGG
GCTCTGTCAAAATTTAACAACATAAATATTGGGCAAATGAAGTTGTTTCT
[C/T]
TGGTGCTATCTCAACCTGCCAATGGAGAAATATCTCAAAAAGTCAAGAA
TTGAAGCCATTACTCAGAGACAGCATCCTATGGCATGGTGATCCAAAACA
AGTACACCTCTACCTTTTCCCTTTCAGCCCCAAACCCCAAAACACTGGAG
AATATTGGGGCAGGCAAGGCCCGTGAATAGGTGAGCAGAGCTGGAAGAAG
A
>rs4571035
AAAGGCTTTGCCAGTAGTGCAGCCCTCTCCTCCCAAAGTCCTAGAGGCAA
TTACTCTGAAATATGGAAGAAAGAGGCACATCTGAAAGCCCAGTGCTGTC
TTCTTGTCCTCACATCCCTCAGGATCTCAAGGTCAGCCTAGTAGAGGCAT
TGCTTTACCATAGTAGCAACTCAGGGAATATCTGTGGAACTGGATTCCTC
[C/T]
GAAGTTGGACAGTTGCTTTATCCCTGCTAGAGGCTCTCACTACTTAGAC
CCACACAGTCCTATAGATAGAAGAAACCTAGAGAGAACCACCTCTCACAC
TTTTATTTTATACATGGAGAACCAAAGGCCTCAAATGGTGGAGTGATTTG
CTGGGGTGCTACAGTCATTGTCACAACTTACATTGTGACATGGGCTTCAT
A
>rs10195963
GCTGCAAGTCAAGTTCGACATTCAACTATTTCCTCGGGGTCCTTTTTTGG
GAGAAGCAAGGCTTTGAGTTCACTCTTTGACTTCAAGTGAAGTCCATTTT
CCTCTTAGACACTACCCCAGTGGTTCTCACACTCAAGGAAGCACCAAAGT
CCCTGGAGGACCCGTCTAAATGAAGGATGCTAAGCCCCACCTCACAGAGA
[G/T]
TGATGCAGTACGTCCCAGGAATCTGCATGTTAAAGGAGCTCTGCAGGTG
ATTCTGATGGAGGTGGCCCTTGCAGGATGCTTTGAGAAACTATATGTTCT
ATCCCTCGAACTCTGCCTGCAGAACAGCCTGAGATGGCTCTTCCATGCTT
TGACCATTTGGCCCTCCACAGTAGTCCCTGAGCTGCGTCTTCTTCGGACC
A
>rs10207736
TGTCTAACCCCCCTTTCATCTGGTTATAATGAATGCCATTGCTTGTGTAT
AGTTGCCTACATTAATAGACAACAATGCAATATTCTCCTTATGGAAACCC
TACCTAGATTGGGAGGCACCATGAAGCCACTTTTTCTTCTTTCTCCATCT
CTACCCTGATTAGCAAACATCTCTAATTATTGTGACTCCCAACCCCAGTC
[A/T]
GATCATTTTATTCTTTTTCCAGTATCTATGCAGGGAAGCCTGGCAAAGG
CTTAAGGGATGGGGTGGGGTGTAGCATGTCAGCAGGATTTGGAACTATGG
GCCCCATACTTTTGGCACCTGCTGCATTTTCTCCCCACCTGCTGCACCAT
AATTCATCTGCTTGTATGAGTTTAGCAGAAAGAGCCCCCAGTTCCCTGTA
A
>rs12464018
GGGAAATTTCAGTATACATAGAAGGCTTAATTCAAAATGATATAATGTCA
ACATTTGGTATAAGAAATTATTTATCTGGCAGAATATATACTCACAGATG
TCAAAATGGGTCTAGAGAGTAGAGAATGGAGGCAATTATCCAGACACTGA
GGCCCAAGGCACAATACTTTTAAGTAATCATGCCCTCAACTGGCATTTCC
[A/G]
TAGTCTTAAGCCCAAAAGCATTGGGTGGAACCATTCTATAAGCAAAGCC
TCAGCTATGAAAACAACTTTTAAAATTGACATTCAAAAGATAAGATCAAC
AACAGGTGCCCTTTGCTTTCGGCAGTAGGTGTGTTCTTGAAAAGTTGGTT
GTCAATATTTCCTTTTCCCATTAATCTAATCACATTCTAAATATAACAGG
G
>rs10932689
GGCTCGAATTATGCTTGCACTGAACAAGGCTAGTCTAAGGTATGCAATTG
CCCTGCTTCTTCACTACTTTCAAGTGTAAACAAATATGTCAGCATTATCC
CTAGAAGGAGGGTTTAGGGGTTTTAAGTTGAATACTCTTCAGCTGAAGAA
AATCCATTAATCTCCAGTTTTATTCTTGAGTTAGCTTTCAAATCTACATT
[G/T]
CGACAATAATTACTTTGGGATCTTTGAATTTTCTTTCATGCCTTTCTGT
TCTATGAAGCCCACATGCGTAGTTTATGCATTTTCCAATTTAGGCTAGAA
GTCCAATGTGCTGTCCATTGCACCACAGAGCCTGTTTGCTATGCATTTTT
AAGAGTAAAATTTTCAGAAAATACTTAAATTAAAAAGATAGATTTGGAAA
G
>rs12613030
TTAAATTAAAAAGATAGATTTGGAAAGCATATCCAAAATGTTGCTTTGAA
CTTCTTATTCTGGACAATTCCTGGACTGGGGAGAAGGCATGCCCAAGTAC
ACATTGCTTGCAGTGGCACCTCCAAACCAATGCTGGTCGGGACTCTTCCT
ATTACCTCGCAACAGCAATGACTGGGCCCCATAATACAGCCACGGTCATT
[A/G]
TAAAAGATCATTACAAAGCATCAAATTAACTGCTATTAATGAAAGCCAG
AAACACGTCATTCAGGAGGTCGAATGGCTGGGATGGAACATACAGGGTTG
AATAATGGTAGGAGCCTGGGTTTGGAATTAGAAAACTGGGACAGGAATCC
TACTTCTGCTATGAATCCATTGCAAGTGACTGGGCAAGTTGTTCCTCACT
C
>rs2888450
CAGATCTCAATCCGTCTTATCCTACCCTCTTCCTGACCTCTAGCTACAAG
AACAGCAAGGCACAGCCCCCTGGTTTTGAGAATGCTAATGCTTGCCAGCA
TTCTTGTCCTGCAGGGCATGAAGTATAGTCCTGCACAACCAGCAGGCTCT
GTGACTGCCTAACCTCATCCTTGGTCACTGACCTCTGTCTTAGTAGCAGT
[A/T]
CGTGCCATCCTACCTAGCTTTGCTGGGTCAAGTGCGTTCACATCTTAAA
ACCGTTTGAAGCGTTTGTCTAATTAACATGCAGAAAGCATATACTGATTT
TTATTTTCACCACCATGTATGTGAGGGCCTATTTTATGGCATGCAAAGCA
TCTCATTTTTATTTCAATTCTCTGTCTTGTAACTTTAAATGAGGCTGAAA
T
>rs4254482
TTTTCCGGTTTGTGACTTACCTTTTAATTTTGCTCATAAATTACTTTTAA
CTTACAGATAGTAATATTTTTGAAAGGTTACATTCATCAGTCTTTGTATT
GTTAAAAAGAGTCTTTATTGTTATACTTTTAAAGTAGCTTTTCCACTCCT
AGTCAGATACTTTCACATATAATAGTTGTCTACTTTCCTCCTGAATTAAT
[A/G]
GGGATTTATTTTGGCATATGGTGTGAAGTAAGAAGCTAAATTCTTTTTC
TTTTCTCTGCATCATCAATGATCTCTTACACTGTTAAATAAACTAGTCTT
TTCCTTCTGCTTTCCACACCCATTTAACCTAGCCCACTGTTGACATCGTC
AGAGAAAACTTTTCATCAGAAAGTCTGAACAGCACAATTATACTGTATCA
A
>rs2372932
TTACCTTTTAATTTTGCTCATAAATTACTTTTAACTTACAGATAGTAATA
TTTTTGAAAGGTTACATTCATCAGTCTTTGTATTGTTAAAAAGAGTCTTT
ATTGTTATACTTTTAAAGTAGCTTTTCCACTCCTAGTCAGATACTTTCAC
ATATAATAGTTGTCTACTTTCCTCCTGAATTAATGGGGATTTATTTTGGC
[A/G]
TATGGTGTGAAGTAAGAAGCTAAATTCTTTTTCTTTTCTCTGCATCATC
AATGATCTCTTACACTGTTAAATAAACTAGTCTTTTCCTTCTGCTTTCCA
CACCCATTTAACCTAGCCCACTGTTGACATCGTCAGAGAAAACTTTTCAT
CAGAAAGTCTGAACAGCACAATTATACTGTATCAATCCAGCTTAAATTAC
A
>rs4255939
TGATCATCGATATACATGATGTCAAAAATTTGGGTATAACTGAAGAATTT
TAGTAATTTAAAGATTTCAGAATTTAAATAATGAAATATCTGATACATTC
AATTGGAAATTTGGTTGAGATCAGCTGACTTGTTAATTGGACTTCACTGA
TAAACATTTGCCTTAAAATCTGTAGAATCTAAAGCAGGGTATACCTAGTA
[G/T]
GCGAGTAAGACATCATTTCTGGAATCAAGGGGGCTTTTCAGTGTTGACC
AGATGTTTATCACTGCATGGGCAAATGGCCCAAGGAGTATGGTTGTGGCT
TGGCTTATTTCTTCTTAACGCAGATGCAGTTGCTTTCTTTCTGGGTGGCA
GTGTGGAGGATAACTGGGGTCGGTGGGGACATAAAGTCTCACAAGGGGAG
G
>rs10192415
TTCTTTATTGTATCCAGTATAACATACTCGAAGATGTATATATACACATA
TATAGATATACAAAGCATCTATACATCTAGACATGTATATATACATATAT
ATATACACACACGTATATACTGTACTTCAATCATCTATATTAAATTGTGA
TAATATCACATTACCACATGTTGATGGCATTCTTGTAATCTGCCTGAAAT
[C/T]
CATGAAAACACATGCAGTGGTCTGGCTCATGTTGTCTCAGGGAGTGCAT
TCTGGCTCCCAGTGATCATCAATTACTTTTATAAAAGTTCACCAATCACT
TGTTGGTTTCCGGAAAACAAATTTAAAGCAAACATTATTAATGTCAAACT
TACACATTTTGCTTTCTGGAATCAACTTTTTTATTTTAAAAATTGAGAAT
A
>rs7595393
CAGGGTCCACGGTGTCCTTCGTGTCCCTCCCTGCCCAGCCTCAGCAGGAG
CCCCCTGCTCCTAGACCACTCCCTTACTGAGTCCTGTAGATGTTCCAGCA
CACACAGTGCCTCCCTTCTCAAGGCCCCAGCCCCAGCCCCTTCTCAGGTC
ATTTGGGGGCTTACATCACCTGCTACCTGTGTGGAGATGGAGGTCTGTTT
[C/G]
CCAGCACTGCCTTGCTTTTCCTCCTGCCCCACTGCATTTAATCACTGTG
ATAAGAGGAAACAAAGAGGGAACGTCATTGGTCCCATTCCTCTGAATCTT
GACCACGAAAATATCTTGAATTGCCTCCGTGTCCCCACCCAACATCGCCT
CTTGCACTGCTACCTCTGCCTGAGTGCACATCAGCCAAGAGCCAGGAACC
A
>rs2372935
CCCAGCCTAATGTGTACTTTTAAAAGCACTTTTCAAATACTTCATCTTGT
TTAATCTTCTTGGCAGTTCTGTAAAGTGTGAAGCATTGTACAAAGTTAAC
TCTCATTATTATATTGGAACCCAGGGTTTTCAGAGGAGAATCCTCAAGGA
GATAGGGCTTTCCTGCCTTGTGCCCATATAAGGTTGTGGATCCCAAGGGA
[C/G]
GAAAGCATTTTCATCAGACTGGGAGAGGCAAGAAAAGGTCACTGGCAGT
ATAGATTCTTCTGGAGTTCACTGGCAACTTGTCCCACTTCAGTTCTCCCT
TAATCTTTCCAGATGTGCAGATGTATAGCCTGGGTGGATGAGCCAAAATG
AATTCAAGGCTAAACAGGTCAGCTGGCTCAGCCCATGGGAAGCATGCTCA
T
>rs6733648
TGGAGAAACCAAGAGGTTGATTTCCTCAGCCTTCAAGAGAGCTCATCCTC
AGCTCTCTAAAGCTGACCAGAAAGAGGAGAGTGGGACCTATGAAAACAGC
AGGAGATGGGAGGGCTTGAATTCCTAATGCTATACCCATAGGTCATTGTG
CCATGGTCCTCTGCACCCCCAACACCCCCAACACTGATCCTGAAGGTCTC
[A/C]
ACAATCTTCATCTGCCTCTCTCCTTTCACCTTCTTTACACTCTCATTGA
TAGCCTTGGTTTTTTCTATACCTCCAGTATTCCCATTCCCATGATAGCCA
TTTAAGGACAGGGCACTTCCTGGTCACCATAAGGAATATTGTGGGAATGT
TCTGCTCATGGATATTGCATCAGAATAGGTCTGGAAATGAAACACACCAA
C
>rs6435957
GAGACAGATGGAGAGTGAGAGAAGGGAGGCAGGTAGGTAGGCAGAAGAAT
GAGGAAGAAAGCAAAGGAGTTGAGACTGGGAATGGACAGAAGGAAACCTG
TACATGCTGTGATCTGGGCTTTCTATGTTCCCTGATCCAGGGTCCTGAGA
TCTTGGGGTACTCGCCACTGCATTGTAAGTTTCTGTGGATGAAGCTGGCA
[C/T]
ATAAATACATCTGTAAAGCTGTAACTCCTGGGCATTGTCCCTGAAATTT
TACTGATGTCTAGATTCTAGGTATGGGATTAAATGGGTCCACATTTTGTG
TTTTTCTGAGTCACCAATTACAACAAAACTTGCTCATATACTTATGCCTA
TCTTAAAAAATAATTTATTTTAATAAAATAATAATAAGGCAAGAAAAAAA
G
>rs6723019
TCATTATCTCCAGAAAGCTTTTCCAGCTTCCAGTCACTGCTTCACTCCAG
TTTTCTTTCTTCTTTTTTCATCTTGGCTTATTTTAGTTGGCTTCTATGCC
CATGACTATCACCCTCAACCAAAGACAGAGAGGCTCCAATGCAGGAGGCA
GACAGATTTGGGCTTGAACCCAATGCCGCTTCCTTGCTGTGTGAGCTTTG
[G/T]
CAAGTTATTCTATTGCCTAATTTTTGGGGATTGTATTATGTTATGTTAC
ATGTTATGTTTTGTTACATGATATTGTGTTATATTACTATCTTCTTCATG
GTGTTATTATATTATTAAGATTGATGAGATAATGTGTGTCAGATTTCTAG
CAGGTTTCTTGGCATATTGCAACCACCAATACACTGAACATATACTGTCT
A
>rs12614773
CCACATGTTTCTCATCCTCCTCCCAGGATTAGTGGGCAGCCCAAGCATGT
CTTTCTTCAAAATGATGCTGGATACATAAGAGAGCAAGCAAAAACATACA
TGGCTTCTTGAAGCCCAGGCTTGGAATTGGTATGCCATTGCTTTTACTGG
CCAAGCAAGTCACATGGGTGGCATCAGGGATGGGCTTATGCATCATCTAC
[C/T]
GTGGGAGGAGTTTTCTAAGTATGATGAGGGCACAGATACAGGGAGCGGT
GACCATTACGAACATTAATGCGAATCCTCCACACTGTGTTTCTTGATCTG
CCAGGACAGGGTTCTCTGAAGGCCAGTAGAGGTTCAGTGCTTCCAAAAAA
GGATGCTGGGCTCAGGTAGGTAGACAGAGTTCATCACCTCTATCATGACT
G
>rs17833842
TAACTTATAAATTGACTACAATCCTAATAATCACATTAAAATTTTTTATA
GTGAAATTGATACTGAAGTTATATGGAAAAAATAAGAATAACTAGAGAAA
TATTAAGAAATTAAAAGAGAGCTACAATGAAAGATTAACTCTGCCTAAAT
TTAAAAGGCATTCTAAAGTTTCTGTGATTGAACTAGTAAGAACCTTGTGC
[A/G]
TAAGTGGAAAGACCAACTAATGAAATAAAATATGATCTGAAAGAGACCC
ATATGCATGTGAAAATTTAGTATATAATAAAAATGCAAGTTCTAATCACT
TGATAGTCATTCCTTTAAAGAGATACAATGAAATCCATACCTTAAACCAT
ATAAAAGAATAAACCTCAAATGTATGAAAGATAGTATATATAGAAAATAA
A
>rs10177578
TGACTAGGTAAAGTTTAGAAACAACAAATTTGAATGTTCAAAAATATTAT
CAGCAGTAAAGTCAAAAGCCAAGTGACGAACTGGTAGAAAATGTTTCTAT
ATATATCAGAGATAAAAAGTTAATGTCCCCAAAGTATAAAGAATTTTTGA
AATTGAGAATAAGCGGAAAACCAAAAACCAATATAAATGGGCAAAAGATA
[C/T]
GTTCATATAATTTAAGTGGTCCCTAAATATATAAAAATATCTTCAACTT
TCAAATATCCTTAAACATATAAACATACATTCAAATATGTTTCTTGTAAT
AAGAGGAATGAAAATTAAATATAATGAGATACCATTTTTCACCTATCAGA
ATGGTAAAACAATTTTTTTTAAACTCTTAACACACTGTTGACAAAGCTGT
G
>rs12623304
AAAAAAAAACTATGAAAAAATGATGGCGATATATCAAAAAGACTCAGGAT
CCAGTGTGAGCAGCTCTTATTGGGCAAGTCTTGGAAATTTGAACAACAAA
ATCAACAACGGTTAGTTACACATTAACATGTTTGACAAAATAAGAATCCA
TGAGTCCAGACTGATATAAATTAATGAGTGAATAAATGAATAGGTGATTG
[A/G]
GAAGAAAGGAAAGCTTCTCTTCACAATAGAATGCCAATTAACAAATACA
AAAGGAACAATGGAGTTAGAAAAGAGTTAGAACACTACCATTTTGCAGCA
ATTACAATAATAATTAGTTTAGGTAAGAATCATGAATGGGTGATACATTT
ACGGAGACTGTTTGATGAGGAACTGGATATTACATAGCCTCACAGTGTTT
C
>rs6716542
AGAACACCTTCCGCACAAGCTCTTCAAATATCTCAAGTTCTCTTTTCCAG
TATGGTGTTGCAGGGTGTAGTGAAAAATGCATGAGTTTGGAGTCAAACAG
ATTCCCGTTCCATTTCTGGTTGTATCCTATATTACCACTTGGAGTTGTTG
GCAGTCTTAGGTGAACACAGGAGGCAATTCTTTTTTTACAGTGACTGACA
[A/G]
AGAGGTGAGCAAGAAATGTGAGTCTCTTATTTATTCCCAGCCCCTCTAT
CTTAACTTGTTGAAGGTAAAGACCAGTGGATCTGAATTTAGATTATTTAC
CCTCCCATGTGTGGGGAACACACACACGTGCACACACACACCAGGCTGGA
ATTTCTTCAGTTTGTTCCTCCGGGTCTACTCTATACTTCATGGAAGCTGA
T
>rs17777330
AGTCAGCAACATGTCTCCAATTAACATGACAGATTTTTTGGACCACAGAT
CTCTTTGGGAATCTTATGTAAGCTGTGAAACTTCTCCCTTAAAAAACCTA
CATATTTAATTCTGTCTGTAATTTTAGGACTTCACATATCCCCAAAGACC
ATTCATGATCCACCAGGGTTAAAGACTTGTTTTAAGTAAAGAAAATATTC
[A/G]
CAGCCTCTGGAGTAGTAGACATCTCCAGAGAAAGTCTGGCATTCTTTGG
TAACTCATGGCATTTATTACATTAGCTTTGGATGGAACAACCCCCAACCC
CCAAGTCTGAGCATGTGGTTTGATGCTGCTTCCTTTCCATTTTCACTCTG
CTCTCTTGGATTCTATGTAGCAAAAAGGTACCTTTTCTAGGGTCACATGT
C
>rs10211546
ATATTTCTAGGGTAGTTTAGGTAACATAGAATTTGTAGCTCAGCAGTCT
TGGAATCACTAGACTCTAAGCCCCATGTAGTCAGAGACTGCCTGCTTTTA
TAATTCATCCCCAACAGCAGGCTATTGCATAACACATTGTTACCACCCAC
ACCATTTTGTTGAGTAAATGAATGCATGCATGAACGAATCAGAGGATTCT
[A/G]
TTGTCTAAATAAACTTTGGAGGTGACTTAGTCTTCTCTTCCCTCTGATG
CTTAGATCTTCTTTATCTCTCAGTTCAACATATATTGCTCAAATGGTTCC
TAGGTGCAAGGGATCTGGAAGTTATTAAAATGAAATAGATTTTGGACCAT
GACCTTGACAAATTATGATCTACTAGGAGAGATAATATGGAAATCAGTAT
A
>rs2372937
GAAGTTATTAAAATGAAATAGATTTTGGACCATGACCTTGACAAATTATG
ATCTACTAGGAGAGATAATATGGAAATCAGTATAATACTACAGAGACAGT
AAAAAGGAAAAAGAAATGTTTGCACTTAATAATTATTCCACCATTGTTCC
AAAAGTTTCAATTATAGGGAACTAAGTTCCACTACTAATAAAAGTCAATA
[C/T]
GCATTGTATGCTTACTGCATGCTAAATATATTATGTCATTATAATATTT
ATGTTCAGGTGTATACTATGATTGTATGTATTTTACGACAAGGAAATTGA
GGCTCAGAGAGGTTAAAGAACTTGCTCAAGGTGACAGAGCCAGTAGGTGG
TAGAACAAAGACTCCTACATTCTGACCTAGTCCATATGTAAGCAGATTTA
T
>rs4315498
TTCAATTATAGGGAACTAAGTTCCACTACTAATAAAAGTCAATACGCATT
GTATGCTTACTGCATGCTAAATATATTATGTCATTATAATATTTATGTTC
AGGTGTATACTATGATTGTATGTATTTTACGACAAGGAAATTGAGGCTCA
GAGAGGTTAAAGAACTTGCTCAAGGTGACAGAGCCAGTAGGTGGTAGAAC
[A/G]
AAGACTCCTACATTCTGACCTAGTCCATATGTAAGCAGATTTATTGCTA
GAAATATCTTGATGTTGCATTGAAATCTGCCTCTCCTTATTTTCCATTTG
GAACTAATTCCACTCTCCCGAGCCAGAAAAACCAAGCCTCTCATTACTTG
AAAAACCAATTAATGAGTTAAATTACATCAAGTTAGTTTAACAGTCAGCA
A
>rs12998806
TAGGTAAGTCTTCACAGAGAAACTGGTATTAGGGCTAAGATCTAGCGGAG
GACTAGGGGCTTTCTAGACAGACAATGGAGGCAGGACACTTCTGAGAGAG
GAATGAAGCCTTGAGGTGTGAGACAGCATGTGGTAGAGCATGGAGGGTTC
AGGCACTTGGAGGAGTTGGGTATGGGGAGAAATACAGAGCACCTGTGAGA
[A/G]
CCTGGGGCATGGCTGGAGAGGTAGGCAGGGGCCGGCTTGCAAAGGGCTT
TACCTGTCATGCTGAAGGGCTAACTGCTGTTCTGGAGAATTTGCAGAGCC
ACTGAAGGTTTTGAAGCATCCTAATGTTAGGATTCACTTCAAGTTTTAGA
AAGTTTAGTCTTTTGGCTCAAGGGTGGGGAGGGAATAAAAGAAGCGAGAG
T
>rs12465515
CACCTACTCAGGAGGCTGAAGCGGGAGAATCGCTTGAACCTGGGAGGCGG
AGGTTGCAGTGAGCTGAGTTGGCACCACTGCACCCCAGCCTGGGCAAAAA
GAGTGAAACTCTGTCTCATAATAAATAAATAGAGTGGACAAGGAAATACT
GATGGGGGTGAGAAAACAATTTGAGAGACATTTGAGAAGTAGTGAGATAA
[C/G]
ATTTGGTTACTGATTGAATATGGAGTGTTAGGATAACCCTGGGATCCTC
ATTTTGGGCAATCACAAAGCTACTGGCTACAGCAAAAGGAGGGGAAGAAA
AGTGGTGGTGGGAGGGAGGCATGAGAAATTTGCTGCTGGCCATGTTAAGC
TCCACCTCCTGTCACCAAGTGGTGATGCTCAGGAGATGTTTGTGTCCACA
G
>rs4491709
ACCTCCTGTCACCAAGTGGTGATGCTCAGGAGATGTTTGTGTCCACAGGA
GGAGAGTGATCTGGGTGGGAGGTTTACATGTCCCTCCTTCCATCTCCCGG
TCCTTCTTTCCCTCCTCCCTCTTTCTTTGTTTGGTACTTCCTTTCTTCTC
TCTTTTCTTTTTCTGCCTTCTTTTGAGAATGAAACCTAAGAGTGTTTATA
[C/T]
GTAGAGGGAAAAAGTGGAGCTAGAAGTTTAAGACACAGGAAAGGGAGGA
AAAATGTCCTAGTGTGAGATTCCAAGGAGGAAGAAGGGCCAGGATAAGGT
GGGGAGAGGGATGCCTCTTTTGCTCATTGCCTTTATCTGTCTTTGTCTGT
CCTGGATTGATTGTCCTATCCTTTTCTTCCACTTCGTGTCTTCTCCCGTA
T
>rs2372938
GGATTGATTGTCCTATCCTTTTCTTCCACTTCGTGTCTTCTCCCGTATTT
CTACACAAGGCACAGTCAAATAAGGGCTGGCACGTGCGCATCTTTGTGCA
CACATGTGTGTGCACAAGCATTGAGGGATGCACAGGTGCACACACCTTGT
AGAGCCTCTAGTAGCTCAAGTCACATTTTTTCGCATTTCCAGATTGTAAG
[C/G]
CTTACTGAGGGGCAGATGGGACGTTCTCACAGGAAAAAGGCTGACCTCA
GGGAGAAAGATGTGGCCCCCACCTACAAACCAATGATGTCTACTCCCAAC
AGGGGACGTCTTTCCCCTGCGTGAAGCAACAGGACAGATTTGGTTAGGTG
TGGCATCGATGTCCTAATCTCCTATGAGCACACAATGAATGAAGGTGGGG
G
>rs12621130
GATGGGGCTCTGACCCAGTTTCCATTTGGCCAGCCGACTGGAAACAGCTA
CTCACTGGGCCACTGGGTTCTTTCCAAAGAACAGTGAAGAACAGTGGTTT
GGGGTAGGTGGGGGCAGGGGCTGGTGGGGCAAGAATGTCAGAAGCAAATG
CAGGCGTGGCTGGTCCAGTAGTTCCAGTACCAAAGAAGCCGCTATCTGGA
[A/G]
GAGTGGGACAACCCATCCTAAACATGGGAAGAAGAAACAGGAAATTTCA
AAAGATACAACTACTAATTAAATGAAAGCACTCCTTAACCACCCCTTTGA
TAATTAGATCATCTTCTCCTAGAATGAGAAGGAATATTAGTCTGACTTTG
CCCTCCATATGGAGAAACATGGCAGGCAGGCTCTCTGTGCCTGGAGGGTC
T
>rs13399995
GAAGGACACTGCAGCCAAGCTGAGCCTGTGTCCAGCTGAACACGGACATT
TACTTCTGCTTCTGAGGGGAAATATTGTCATGTCTTATGGGTGTACCCAG
CAAAAGATGTTTCCACTAAAAGACTATAGAGAATTGCTTATTTCTAAGGA
CTGAATTCCGAATGTTTTTGGTCAGGTTCTCTCTGAACAGAGAACCATTG
[C/T]
CCCTGAGCAGAAGCGGTCTTCACACAACTAGCAACCATGTAGTGTTTTC
TCATTCAAATGGCGTTTGGTATTCACCTGCCTGTCCTGGAACAAATGCAA
AGTCAGGGTTGTTTCTAGCTCTCCCCCAATATTGGAAAAATGCTGATGAA
CTGAGCCTGGTTCCATTTTACACCTGACAAAAGGAACTCAGATGAGAGTG
T
>rs12052807
TGCTCTCATGTGTCTCCAGGAGAGTGTGGCCTGGGTTAGCTTAAGACTAG
GAATGTGCTGACAGCTGGGAAGAAAGTCAAAAGAAGGCATGGCATGTGGT
TATGGGGCCGGAGGGACCGCTTGGGGGGAGAGGTGGAAGCGCTGACATGA
GCTTGCTCTGCATCGCTGGTTCCCAGAGGTGGCCTGATAGGAGAGTGCCA
[A/G]
CTGGTGGTATGCCGTGGGCTCTGGGGAAGGGGCTAGAATGTTGTTGCAG
CCCAGAGGGGCTGGGCTAGTGAGGAGCAAAAGAAAACATAAGCTGGGGGG
AAACTGCCTAATAGACCAATTAGGAGACAGGGTTGTGCCCTAAGGGATGG
GTGGGAGCAGGGGGTGCTAGAAGTAGACAGAGAGTAACTCTCAGCTCTGA
A
>rs10199394
GCAAACTCATCTCATTTGCTACTTGTCCTATAGGTAAGGATGGACACCAA
ACCCAAAGAGACTTTTGGGATGTGGATAAAAATTGAAAACTTTGCCTCTC
ATTAATCCCTTGGAGAATATAGCATCTCCTTCAAACTGGACTGCTCTCCT
TGATCCCTTGTCTTTACCTGAAAATATCTTTCAAGGCCTAGCTCATGCTA
[C/T]
CTTGGCCATAGGAACTTTCCTTATCACCTCCATCAATGCTGTTTGTCCC
TCCACTAGAATCTTCTGTTTTGGTACTCCTCTTACTCAGCCTCAAGTTAA
ATTTATGTGTACTGGTTTTAACCCCAGTACCCAGTAAAGGTTCTGAATAC
ACGTGATGAAATAAGTCTCTCCTATCCCATGGCACCTAGTATAGCATTTA
G
>rs2372940
AAGTTAAATTTATGTGTACTGGTTTTAACCCCAGTACCCAGTAAAGGTTC
TGAATACACGTGATGAAATAAGTCTCTCCTATCCCATGGCACCTAGTATA
GCATTTAGTTGCTATACTAATTATTATAATTAGCATTTATTACGGGCTTA
CTATGTGCAAAGTGCCATTATTCCATTTATTATTCACAAAAATATTTAGT
[C/T]
TCTACTATTCTCTAATTTATTAGATAACCTAATATAAATCTTTTTTGTT
TTGATTGTTGCTAGCTGATTGAAGGAAATAGAATTGTGGTGTGGTAGTAG
GGGATGTTGAGTGAGTCTTCCTCATTCTTAATATTTCCTTCCACTAGGAT
AACGCCCCTGGGCTCTCTTCATCAGGGGAGGAGGAGTGACTTCATCTGAT
A
>rs4583440
CCTAATATAAATCTTTTTTGTTTTGATTGTTGCTAGCTGATTGAAGGAAA
TAGAATTGTGGTGTGGTAGTAGGGGATGTTGAGTGAGTCTTCCTCATTCT
TAATATTTCCTTCCACTAGGATAACGCCCCTGGGCTCTCTTCATCAGGGG
AGGAGGAGTGACTTCATCTGATAGATTCGGAAGGGAAAATGTTACCCAGT
[C/G]
GCTTCTCAGTCCTCTTTCTCTTGGAGCGAGCCCATCTCTGGAGCACATA
CACTCTCCCCTGCTCCTGTAGGTCTCGAGTTCTCAGGGGAAGGGTTTGCC
CTGTAAGAGAGCTTGTCCTTCTCCAGTGAGGCCTGTCACTGGGAAGATAA
TTCTGTACAGTGCTGGGATTCAAATTTAGAAAGCCCACTTGCCCCATAAT
T
>rs2372941
TGTGGTGTGGTAGTAGGGGATGTTGAGTGAGTCTTCCTCATTCTTAATAT
TTCCTTCCACTAGGATAACGCCCCTGGGCTCTCTTCATCAGGGGAGGAGG
AGTGACTTCATCTGATAGATTCGGAAGGGAAAATGTTACCCAGTCGCTTC
TCAGTCCTCTTTCTCTTGGAGCGAGCCCATCTCTGGAGCACATACACTCT
[C/G]
CCCTGCTCCTGTAGGTCTCGAGTTCTCAGGGGAAGGGTTTGCCCTGTAA
GAGAGCTTGTCCTTCTCCAGTGAGGCCTGTCACTGGGAAGATAATTCTGT
ACAGTGCTGGGATTCAAATTTAGAAAGCCCACTTGCCCCATAATTAAGCC
ACATTCACCAGCTTTAACAATGACAGAGGTGATATTGAGTTCTCCAGTGA
T
>rs6721811
TACCAGGTCCTTCATTATCATATTCTACACATTGAAAATGAAGGTAAGAG
AGCAAGCAACTTGCCCTAGGCTACCCAGGGAATAGGGAGGAAAGTCTGGA
TTTGAACCTACTGCTGCCTGACTCCGAAGCCCTGTTCTTTCTCTCACCCT
GGAGGATGGATGAGGGTGGGATGGGGCAGTTAAAAGTTGAGGGGTATCTC
[C/T]
AGGAGAAAGATGAGACACATGACCATTGGGTCTAAGGAGCCAGAGAGAG
GCCATTGTGTCTGAGCCTGGACAGCCAAGCTTCCTAGAGACAGAGGATGG
GATGAGATGACTTCTGGCTGACTCTGCCTCCTTGAGCTCTTTAGGATCCC
CTGCAGAATCTCCCAAAATGATGCTCATTGGAAACAGAGAGAGCACATTC
A
>rs12615418
TAGAGATGGGGTTTCTCCATGTTGGTCAGCCTGTTCTCAAACTCCCGACC
TCAGATGATCCACCTGCCTCAGCCTCCCAAAGTGCTGGGATTACAGGTGT
GAGCCACCACACCCGGCCAATTTTTTTTTTTTTAAAATATGGCATTAAAA
TGGTATTTATCTTGACTACTGAGTTTTTGGTTAACCCCTTACATTTTGTG
[C/T]
CCAAGACATCTCACTCATGTTATCCTAGGCCCAGCATGGCTGGCCTTGT
CTCTTAAATTCAAATTTTTAAATTTTTCATTCCTCAGGCTTCTCACTTGT
AAAATAGAAATAATAATAGCATCTCCCCCACAGGCCTGTGCTGAGAGTGA
AATGTGTTACTACACGTGCCTCACCTTCATTTTCAACAGGACCTATTGCC
A
>rs12621884
CACACCCGGCCAATTTTTTTTTTTTTAAAATATGGCATTAAAATGGTATT
TATCTTGACTACTGAGTTTTTGGTTAACCCCTTACATTTTGTGCCCAAGA
CATCTCACTCATGTTATCCTAGGCCCAGCATGGCTGGCCTTGTCTCTTAA
ATTCAAATTTTTAAATTTTTCATTCCTCAGGCTTCTCACTTGTAAAATAG
[A/G]
AATAATAATAGCATCTCCCCCACAGGCCTGTGCTGAGAGTGAAATGTGT
TACTACACGTGCCTCACCTTCATTTTCAACAGGACCTATTGCCAGTAAGT
GCTCGTTAATGTTAGCCATTATTCTTGGCATTGCTGTTGTTATTAGTGTC
AGCCATCATTCTCTCAGCCAAGAGATCCTTCCAGAAGAGAAGTTCTCCAT
G
>rs2372943
CTTGAGTCTCAATTTCTTCAAGTTGGGAACATTACATGACAGGACAGGAC
TGTGAGGATTAAATGTAAACAACATTTTTTTTTTTTCTATAGAACATACT
GTGGGGCCAGCACCATGCAAGGTGCTTTTATCTACAATTTATTTCTTTCC
CTTCCCATGCCTTCTGCCTCTTTGAATAACTGCTGCAATGGCTGTAGAGA
[A/G]
ATAAATGCAGTAGACCAAGACTTTTAGTGATTTTTAAACAATGTTCTAG
GGCAAATACATGGGTATGTTTACATACTCCACAAAGTGAAAAAATTATGC
TAGTCAGTGTGCAAAGTGTGTTTTTCCAGACCTGCAAAAACTGAGATCAA
CTTCCATATTTCCAACTTCCATATTCCCTTCATCCCTTAAGCTACCAGGC
A
>rs4522583
ATTTTTAAACAATGTTCTAGGGCAAATACATGGGTATGTTTACATACTCC
ACAAAGTGAAAAAATTATGCTAGTCAGTGTGCAAAGTGTGTTTTTCCAGA
CCTGCAAAAACTGAGATCAACTTCCATATTTCCAACTTCCATATTCCCTT
CATCCCTTAAGCTACCAGGCAGAGGATGGGAGATAAAATTTGGCCATAAA
[C/T]
TTGTCATGGGTCCATCTTGGTCCTAATTAGAATTATCTACTTCCTAATC
CATTAACTGCATGGATAGATAGTCTGAGGGCCTCAAGGGCAAGAGATGAC
TTCTACCCCTCCACCCATCCAGCCCTGACCCACCCTTCTCATATACATTG
GAGAAAGCTCTGCTCTGTGTAAAACACATCAAGTGCAGGACATGGTTCTT
T
>rs2888451
GTAGAGTTTGGGAAATGAGTAACATGTAGATCTGTGAATAGATATGTCTG
AGGAAGCACCAGGCAGCGGAAATAGCTGGTTGCAGGAGCAGTTTGTCCTG
CCCACCTGAATCTTCAGGTTCCTTCAGCTAAGCCTTAGGAGAGGAGGCTG
GAGAGCTGCTTTGCTGGATCATCTCCTTTGGCCCCCCTCAGACTAGAACT
[C/G]
CACCTCCTCTCTACCTAGCTCTGTGCTCCACAGGCTGCCTCTGGGCTGC
ACTGATGGGCTCCCTTGACTGCTGGCTCCTGTTCAGGGTTGGAGGAGGAG
GATGAAGTCATGGAATAGTGTCCCCATGCTGGGCTGCAGGTACAGTTGCT
GCTTCTCTACTGAAGACCCCTGACAGCAACCCTGTCTTACAGCCAGGGTG
T
>rs2270398
TCAGAGCAGGAGACATCATTTTCCCTTAATTCTATTCTGCCTTGTAACCC
CTTTCTTAGACTCTCTGCTGAGTCTCTGCTTATGTGTTGGGATTGCATTG
ATGCAGATGCTTTAGGGCCACGTTGTGGAGGATACAAGATGATGAGACAG
GAGGCAGCGTATGGTGAATCACTTCTCACAGCTCCTAACTAAGTTCTGGG
[C/T]
ACATGAATGGGTACTCAGCACATACCTACTGACCAAGCCTATGGAGAGC
TGAAAACTCATAGGCTGACTTTGAAGCGAGGCCTTTGAAACCATCGTCCC
AGGGGAAATAATCTAGGTCAAAGAGACGGCTGTGGCAAGGGTGCGAGCTG
GCAAGCATGAGGGCTGACAAAGCAGGCAGCACTCCTCCTGAGCAGATGAC
C
>rs13412666
TATTCCCAGGCTGGGCTGGAGCACCTGAGGCTACTATGGTTACAGAAAGG
AACCTGGCATCCGAAGGTATAGGCTCAAATCCCAGCTCTCATGATTGCTA
GCTTTGAGGTTTCAGGTTCATTTCTTAAGTGCTCTGAGCTTTGGTGCCCT
CCTCTTCAAAATGGGAATGAGGAGCATGTCTAGCTCCCTGTTTTGTTGCA
[A/G]
TGAATAGCTACATGAAGAACAGCTAAACCAGAACAGAAAGAAGGCAAAT
GGAAGCTACAGAAACCAAGGATTTCCTTGTTGAATCGAATCTTCCTTCAA
TCTTCCTTCACCACACTAGTGGATCTCCCTGTGGGAGGGATGTTGAGAGT
GCTCCGTGTTTTTTTTGTTTTTTTTTTTTTTTTTTTTGTGATGGAGTCTC
A
>rs13426489
CAAGTGATTCTCCTGCCTCAGCTTCCCAAGTAGCCAGGACTACAGGCATA
TGCCATCACACCCAGCTAATTTTTGTATTTTTAGTAGAGACGGGGTTTCA
CCATGTTGGCCAGGATGGTCTCGATTTCTTGACCTTGTGATCTGCGCACC
TCTGCCTCCCAAAGTGCTGGGATTACAGGTGTTAGCCACCACACCAGCCT
[G/T]
CTCTGTGTGTTTTTAATCAATAGCAATATGTACCATATTTAGTAGGGAC
TATAAAGAAATTGTGCTAGAAACACCTGGTTATTTTACAGAACTTTGCCA
GTGAGTACAAAAATATGCGTAGCCCACAACCTCTCTCCCCTAGAAAATAC
CTCCTGTGGGTAGAATGAAATGGGTAGGTGTTCTTACCATTCATTAGCAT
T
>rs6713249
ATTAAACCCCTTCCTTATACATTACCCAGTCTCAGCTAGTTCTTTATAGC
AGTGTGAAAACAGACTAACACACTCTTAAAAGTAAATGTTATTACCCACC
TTTTACAGATTAAGAAACTTAATATTACATCAAGGAATTAGTTTATAGCA
CTGGATGATCTGGCCTCAAATCTCCAGTGTGATCAGTATCAAGGTTTTCT
[C/G]
ACTGTATCAACAGAGTTTTCCTAACAAATCATAAGACACAAGTAGTTTC
TCCAACTTAATCTATTATACCCATAATTCACTTGCCTCCAGGGAAAAAAA
TTAGATGAAACAGACTGTAGTGGCTTTGTGTAAAACAGCAGCTCCATTCC
TTTAGCCCTATTCTAAAACAATAGACCATGTGCTGATAGCTAGCTAACCT
C
>rs12620095
GCATTTGTTCCAAACACATTTTACATGGATTATCCCATTTAATCTTTACA
CTAAAGTATAATACTCTTATTACCCCATTTTAAAGAAGAGAAAACTGAGG
CACAGTGAGATTAAGGAATTCATCCAGTTTCTGCTGCAAGTAGGGTGGCT
AGCATTGTAATCTGGGCATTTGCCATGAAGCTGTGCCCTTAACCACTCCA
[C/G]
TATGTGCTATAAGGTGGAGCTGACTTCATGGGGGCTAATTCAACCAGGG
GAGTGTGCTCATATTAACAGATCTGTGAACTCCCTGGTTTGTTTGTGAGA
CAATTATCTATTTGCTTACCTGAATCACAGCAATAATAGTTATACCTCCC
ACTTACTGCATGCCTACTTGGTAGCATGTACCTGGACAGAAGGTTTTCAT
T
>rs4621152
GGCGTGAGCCACCCCGGCTGGTCGCATTCCTCTATTTAGTGTTAATACCC
TAAGACTCCCCAGCTGACCACCAAATGTAGGCAGGTGGAGGGACTGACCT
CCAGGGGGCACATGTCTCAGGTTGACAAACACATTTTCTCTTGATCCTTT
TTTTCAGTATGATGAAGTATTCTACAAGACTCTCATTGAACCTTCTGATA
[C/T]
CACATTCTAACCATTTATATAAAAGTCAAAAGCTCCTTGTCTTCAGGAT
TCCAAAGTTCTTAGCACAAAGGTCCAGGAAGTGTCTGAATTCTTTGGTGT
CTGGTTCAGTATGGATGTTCGGGTCCCTCTTAGACTTGAGATAGAACAGG
AAAGAGATGAATAGACAATGACAGAGGTAGAGAAAGTACCAACCCTTCCC
A
>rs12622764
CCTCTCATCTCCTGAATGGCTGACCAGTGCTTGCTTCAAGTTAGATTAGA
GTTTGAGCTTTGTAACATAAGTCCCCTTCTGTCTGATCAATGAAATGAGG
GATAGGTACTTGCCCGAGAGTTATGGGACCTGGCATTTAACGTGAGAACA
TGCATCTTCAGAAGCACTGGTGAGACTTCAGAATTCCTGAAAGCACTATT
[C/G]
TTGCAAGGCTTGAATGTACCTTCTCCACTCCACCATGTCCTCTCCCAAC
CCTACTAGCATGATATCAAGCTTGGGTGGAGAGCTCGCTTGCAACTTTGT
CAGATACCCATGAGCCACTCTTTGAATTTCTGACAATGAACAAGTATGTG
GGAGGCCAACCCCATCCAACCTGACCCATCTGAACCCAATCACATGTTTT
C
>rs6723013
AGAAGAGTCCTTTGTGACTCTGATTGGCTTTGTGACTTCCTTGGGGCTGG
TGTTGGTCTCAGGCTTGTGTCTCGCAGTCATCCAAAATGTGGGTGGGCCA
TGTCCCACCTTAACTGTGGTTGTGAACGCAGAGGATTCACTCAACTTTCA
GCATGCAACAATAATCAAATGCAGTTTTATGGTTCACAGAGGGCTCCACC
[G/T]
TCATGATTTCATTTGATCCTCCTAACAACCCTGGGAGCTCAGAGAGACT
AAGGAACTTGTCTCATATCACATACCCAGGAGTTGACTGCACGGCATCTG
ATGCCAGCTTCTGTACCACGTGTCCTCCTGGTGATACTAGAGTAAATAAA
ACATAACTAGACAAGTAAATATTTTGTACAAAGTAATCTGCTAAAAATGT
>rs2372945
GAGCATTTTCTCTTGGTAAGTTCTCATTTTCCCTTAATCATTAGAAGACA
CGGCACAGTAAACGCGAGCTCATGAGCAGAAGGCTACATAGTTGTATACA
TGTGTGTGCTTTTTTTCACACCAGAGTTTGATGAAGCAAAATACCCAAAG
GTCTTTTAAAAGTAAAGAGGTTTTAAGAGTAAAGAAATTTAAGAAAAAAA
[A/C]
CAAAAAGAAGTAAAGATGGTTAGGTCTCCTGTTTGCTCTCCTTTTGCAA
TCAGTCTGTCTCGCTCTGTGAGCTGACTATGCTGCAGACTTTTCTTTTAC
CTCCAGCCTTTCGCTTGTAGGGTTTGAGGCTAGGGGCGGGGAGAGAGAGT
TCAGCCCAGCAGGCAGCAAACCCTATGTGCAAACACGATGTGTGAACATT
>rs12694403
TCACTCTCTACCCCTGCTGCCCTCCCAATGCTCAGGTGGGCCCTAGATTC
ATCCAGAAGCCAGGGTGTTGTGCTTCATGAAAGGGCCAATACCCCTAATG
TGGGACCTATACAATAAAAGGCAAGGGCAGAAAGAGAGAAGAAATAAAAG
GAAAAGGGGTGTCTTGAGGGGAGTACATGATAGACTCCTGTGCCTGGAAG
[A/G]
GACTAGGAAACTTATACTGACTGGTTCACTGCATTTGCTCAAAGAGTAT
TCATCTTCTTGAGTTTGTCAGGTGACTGTAAGAGCTGGGTTCAAGTTCTT
TGGTTCACTATTATATGCCTAGCTGAGCACAATGCCTGGGACTTAGAGGG
AAGGTCTGGGACTTCACCAGGATTCCCAAAATAGAACAAAGCATTTGTTA
>rs12613955
TGAAATATGCCAGCCAGCTTAAATTAGCAAAGCTGGAACTGTGGCAGAAT
GAATAGCCCGTGTCTGAGTGAGAGGGAAGGAAAGGGTCTACTGTGTGATA
GGCAATGGTTCCAAAGTAGGAGTAGGGGCCTAGTGTGGTCATGCCTTCTG
ATATGTCAAGAAAAGGCAAAAAGTTGATATTTATTTGAAATATTCTGATT
[G/T]
TATAGGTTGGTGATCAATTAAAAAACTAACAAATACAGTTAACAAACAT
GTTTTAATGCTTGCAGACAGGATCCTTCCTGCCGATGTTTGGCCTTTGTC
TTTAATGGAATTTGGGCTGCCCCATAACAGCCCACCCTAAGCAAAACTTC
TTTTCTGCCTTTCTTTTATATTGTTTTTTATGGAATGTAGAGGGATTTTT
>rs11894340
CTGCTTCTATGCTGTATTCTAGTCATTAAAGGAGACCATCTCACACTCAA
GGGGAGAGACTACACAAGCCATAGTGCTGCCATGGCAACACAACCATTAG
GATGATGCCTTGAGCCTTCCTTCTTCATATTTCTTAGACTTCCTTTCTGA
CCTAGAGGAGCCAGAAGGACTTGGGACTTGTCATACCGTCCAATCCCAAG
[A/G]
AATGACAGGAATGAAGGAATTTTTAAAAATTCAACTCATTTCAACACAC
ACACAAAAAATGAGAGAAGTGAGTCAATTTGAGAACTGAAATTCTGGTAG
ACTTTTGGGGAGACACTAGAAGGGCCATGTTCATGAGTAGGAACCATAGA
TTCTGAGTCAATTGGGCATGAAAGTTTTTCTCCTGGTGTCCCGATCTTGG
>rs17778329
ATTGTGTTGGGGGAAGGGAGGCTTCTGAGACAGAGCTGAGAGTCCCCAGC
AATGCCATCCCTGGCTTCTGGCCCATCTGAATGCCTTACTCTTTCATGTA
AGCAAGACATGAGGTCCTGCTGTTTGAGTTGGCTTTGCCCTGCTCCGTGA
TTTCAGTTCCTTGTCTCTCCAGCGTGTGGGCTCATTTCCGTCCTCTGAGC
[A/G]
CTATTGTTTTTCTGACTTGAACAGTCATCCAGAAAACCTCAGTAATAAG
GCTTTTGTATCATTATTTACTTTGCTAATTATATATGTCTTTCCCATCCC
CAGTGCTTGAAGACATCAAATTGCAGTTGAAGTAACGGCAGAGGGAAAAG
CAAGGGCAACAACAACAAACCACTTCACTCCTCTGGGTGGAAACATGCAA
>rs4442975
AATTTTGGTGATGGGAGGTGTAGAGGGTAAACTCTTGATGATATCATTTG
TGTACCTGGGTCCAGCCATGCCTGAACTTTTCAATTACTTGACCCAGTAA
ATTTCCTTATTCATTTAAATGTTAGAGTTGAGTTTTGTCTCTTATAACTG
AAAGAGTGACATGTGTTTATATTAAGTGCAGGTCACTAATATAGTCTATT
[G/T]
GTTTCCCCAGGCTTGTTCTCCTTTTCTCAAATAGGACTGATCAGATAGT
AAGTGCTCCATACCTGTAGGATACATAAAAGAGCAAATGAACAAGGAGCA
GGAGATATGGTGGCTGACGGTTGGACTATAATTTTTTCTTGATTTTTCTG
ACTTTATTTTGGCTGTGATCTAGGAAGTGAAATCCAGGTCCTTCTAAACC
>rs10932693
TTTATTTTGGCTGTGATCTAGGAAGTGAAATCCAGGTCCTTCTAAACCCC
AATTTAACTGTGGCTCCTGAGGGAGATTTTCAGATCTCAAATGATCTGTA
AGTTTAAGGGAGGATGATTGGTCCCTGTACACTCTCTGTGAGAGTTCCTT
TGGAATACTGGTTTCAGAATGTACAGCCACACAGGTAACTGGATTAGGGA
[C/G]
GGCCAGTGGTTTTTTATGAATGCTATTGAAGACACTGAATGGAAGCAAT
TCTCACCAGAACTCTTTTTGCTGTCATCTTTCAGGGGCTAAGTTGACACT
AGAACCAATCAATGATCCCTGAACTGGGAGAAATGGGTGGCCTTGGCTGT
GATTTTTGGTTGTGCTGCACATTTCAAGATGACAATATTGTTTTAAATTA
>rs17778427
TCATCCTGTTATCCATAATGAAGGAAGATTTTAGGATATTCCAGCTTAGA
TCCAGGTGTCCCCAGCTTCTATACTCACTCTGAAGAAGTGTACTTTATCT
ACTCTTTTTACACCATTTATCCCATTTTGGCCATTTCTGAAAGGAAGATC
CTAGAAGCCAGGCCTCTTGGAAGAAGTTGGAGCTACAGCATTGATAGGAA
[A/T]
ACCTGACACCTAGAAAGGCAAGTCAGTTCTTCCCATGCAGTGGCTTTGG
GGACTGTAATTTAACAAAGCTCTTTTTTACTTGTGTAAAGTGATCTCCAT
TCCACTTTTTTCTTGCACTGATTCTAATTAAGTGGAAGCAATTGTTATCA
TCATTTAGGATTATAGTTAAAATACCCCTCAAATTGTTTCCATTTGGAAT
>rs17835044
AAATATTTCTCTTGGTCACTTGAGTCTAGCATCTAAGTCATATAATGCTA
TTATAGCCTTGTTTTGCTTTTGATTTAGGTCATATCTTCTTTTTCTGATC
TTTCTTAAAGTGAAGGGACCATTTGCTATGCTCACAAAAATTCTCAAGGC
TGGAACTACAGCATGCTCTGGTTTCTAAGCCATACACATCCAGGTAGAAG
[G/T]
GTCAATTTAATTGCCAATGATCTCAGCCTTTTCTAACACTGTTCCTTAA
GTTTCTTTGGCACTGCATGGCAAGCAACTGAGTCATGCATGAAAATTTCT
CTTCTAGACAACCCATACCTACAGAGGGAGGATTTGGTTTACTGCAGATG
CTGAGTGCTTCTGATGGCACACATTCCTTCTTATTAATATCCCCCTGGCC
>rs7562029
GAACAGTCAGCATTGTCTCCGCTTCCGCTCCACCCTGCAGTTCTGAGCTG
AACAGATAATTTATCTGTGGCACAACAAAGTATATGACTTGGAAGAATGT
TAAAGATCAAGTCCACCTTCTCCTTGTATTATATGGCTCTGAGAGCCAGA
GAGGGGAGGTGACCTTCCCAGAATGTGAATGTGGTAGAGCCTAGCTTACA
[A/G]
TTCATGTCCAACTTAGGTGTGAAGTTCTATGAGACTATGCTGCCCTCTG
CCTCACAATGCTTTCTCCCATGGGAGAGGAGGCTCAGAGGTACAAAGCAG
CTGCAGAAAGACTCTCTCAAGCTATTGACTCTTGGATGACAACAATTAAT
GTTCAACTTGGCTTTACCCCCAACATGCACTGCACAGATGCCTGCAGGTC
>rs13000023
TGGCTCTGAGAGCCAGAGAGGGGAGGTGACCTTCCCAGAATGTGAATGTG
GTAGAGCCTAGCTTACAATTCATGTCCAACTTAGGTGTGAAGTTCTATGA
GACTATGCTGCCCTCTGCCTCACAATGCTTTCTCCCATGGGAGAGGAGGC
TCAGAGGTACAAAGCAGCTGCAGAAAGACTCTCTCAAGCTATTGACTCTT
[A/G]
GATGACAACAATTAATGTTCAACTTGGCTTTACCCCCAACATGCACTGC
ACAGATGCCTGCAGGTCTACAATGCCGAGACCAAGTTTGGGGGGCATTTC
CGCAGCTCTGTCTATTGTCCTCCTTTCCTCTTCTGTGGTCATTCTTCCCA
AGGGATAGGCCTAGTCCAGGGTGGAGAAAAACAATTCTTGGTGGTAGGGG
>rs735361
GGTTTATAAGGCAACTCTTAGCTTGACCCACTCTACTCCTCCATCAAAAT
AAAGAAATAAATAAAGTTTTCAAACTAAAGAGCATTAAAGAATGATAACT
TTTCTTTTTCTTGGTACTATCTATTGACCTATACCTGGTTTTCACTGTAA
CACATTTGTGGTATGAAAAATGACAATATGATGTCAGAGAAAATGAGAGA
[C/G]
ACATTTTAGAATTTATCAGAATGTGACTATGGTCACTTAAAGTCCAACT
TTCTCATGTGGGTTTCAGCCTGTGCTGGAAACAAATAGCCTCTCTTACCT
CTAAAGTACTTGGTTCTGTGCTCAAAGTGGCATATTAAGTTCTGCTGATT
ACATCAGAATTTGGATGGTGTCTCAGGATCTCAGTTTCCTATCAGTGGAA
>rs13409592
TTAGAATAAGACTTCCCTAAGCAAATCAATGTGATGATTGATACAAACAG
CCATCACTGGGGCCTCCTCTATTTCCAATCTCTTCCTCAGTTTCCCAGTT
CTTTCAGAACCTCACTGGGAAGTTCAGGGATATATGCCTGCAGGCTGGCC
TGGCGCAGATGGATGATGAAGGGTCCTGCCTTGAGTGTAGGGTAGAGTGA
[C/T]
TCTGAGTGCTTTCCAACAGACTGTAAATTAAAGACACTCCAAAAAGCCC
CTGGTGGGGATAGATGGGGAGAAGGGGTATCACTAAGTCTTCATCTTTTC
TTCTAGGGCCAGGGTCAGTGGTTCAAATAAACATGGTGCCCCACAGAGAT
GGCCTGTGGCCAAGTAGCTCTCAGTGGGTTGACTCAAAGTCACCTGGACA
>rs2287289
GATCCGGGATCTGGTGATCCTCGGCAGGACAACTCACCATTCTGTTCGGT
TGCCCGCTTGCCTCCTCCCGCTGGCTCCAGCTTCTGTGCCTACAATGAAG
GCTGTGCCCATTCCCTCACGTGCCCGCCGTGGCCTGACACCTTCCTGGGC
CCACCCTAACACATTCCTCCGTTGCTTTTAACGACTGTTTACAGCTCGCT
[A/T]
CCTCTAGGACGTGGCTTCTGATGAAAACTGTCCAGACGCTCTGCCTCCG
GTGTAATTCAGAGCCCAGCTCTCCTCCTTAAAATTCTTTTACGCTATTTT
CCACAAGCATGTGCTTCTGTTGGCAGTGCCATAAGTGTGATTGATCTTTA
CCATAAGTTCCTTGAACTTTGCTGTGACCTGGATGCTAAGGTGGCGGGAG
>rs12329133
AAAGTCCACGGGAGACACAGCTTCAGCCACCACCGGGGCTGCGACCTGTC
CTCAGGATCCTCCTCAGCCCCCTCCAGGAGTCCTCAGCTTTCTGCTTTTA
GCCCCAAACCCAGATTCACTTTCCCAGGCCATTCACCGTCTGAGGCAGCC
CAGTCCGCTTTGTCCTTCCTCCTTCCCGAAGGCTCTCAGCCCCTGGGCAG
[C/T]
ATCAGAATTTCACTTTGATCTCCTTTTCCCTGGGAGCCAGAATAGCTTA
GTCTCTTCAAAAGGAGAGTTAGAAAGTGTGCCGTTCATCTAATAAAAATT
AACTCCTGAGAGTTATCCAATCCCAATACAGGTCATTTCGCTGGAGGCTA
CCCCGACTAGGAAACACATTTTAGGACATCAGCAGAGGTAAAGATGAGAG
>rs13011060
TAAATTTCAGCACATGCTATATAGTTTTCAGGATTAGTCCCTCTCTAGGT
ATTCATCTGGGGCTGGCGCCTCCTTGGCTGGCAGGGGCTGGTGATCGGGA
CTCCCCAGCGAGACAGATTGCACCCAGAGCAGTGAGGCCCCGAGCGCTTC
CTGGAGCCTGGGGAAGATGGCATTCATTTTTAAAGTCCATTTATTGTGCA
[A/G]
TTGTTGAAAGTGGGACACAGAAATTTGGACTGATATGCCTAGGCCACAT
GACTACTGATTGGGCCAGGGTAAATGCAGTCTTTTTGTTGATGGAGAAGG
TCTTTTCTGGGCCCAGCACAAAGCAATGGATCTTCAGTCTTAAGAAATGC
ATTGAAAACCATCCTTGTGCAGTTTGGGGTCCCTGGTAGGCCAAGAGCTG
>rs13011326
ATATGCCTAGGCCACATGACTACTGATTGGGCCAGGGTAAATGCAGTCTT
TTTGTTGATGGAGAAGGTCTTTTCTGGGCCCAGCACAAAGCAATGGATCT
TCAGTCTTAAGAAATGCATTGAAAACCATCCTTGTGCAGTTTGGGGTCCC
TGGTAGGCCAAGAGCTGGAAAGGCCCTTAGACTCTGAGTGCTGTAGCCCC
[C/T]
CTTCTAGGTGATGGTCAAAACTTGAAAACGGCTCTAGACTCTGGACAGA
AATGCATGAGAATTGAGTGTTCAAAGCTTGGTGCTTTGACTTCTTTTAAC
TTTCTGTAGACTGAAATATGAATAAGAGGAAGGCATGAGGCCCCTCTCAG
TTTATGGAGCCATAGCTCCAAACCTCCCACTTTCCAACCCAGCAACCCTG
>rs4674132
AGAAATGCATTGAAAACCATCCTTGTGCAGTTTGGGGTCCCTGGTAGGCC
AAGAGCTGGAAAGGCCCTTAGACTCTGAGTGCTGTAGCCCCCCTTCTAGG
TGATGGTCAAAACTTGAAAACGGCTCTAGACTCTGGACAGAAATGCATGA
GAATTGAGTGTTCAAAGCTTGGTGCTTTGACTTCTTTTAACTTTCTGTAG
[A/G]
CTGAAATATGAATAAGAGGAAGGCATGAGGCCCCTCTCAGTTTATGGAG
CCATAGCTCCAAACCTCCCACTTTCCAACCCAGCAACCCTGTGTGTCCTA
GGAATTGTAGCGGGTGTCCCAGAATGCAAAAATCACTCTGCTAGAACTGG
AAAAGGAGACGCCTGCCTTTTCTAGCACGATCTTTAGTAAATAGATTCAG

TABLE 28
Flanking sequences for rs3803662 and correlated
HapMap SNP markers with r² > 0.2

>rs3803662
TTTTATTGTTCTATGGTTATTAAAAAATAACATGTCATATAAATTAAGCC
TAGAAACATGGATGTTTATCAAAGAAAGGATTGTCATCCAAAGCACCAAC
TATGAGAGATATCTATGTGCAATGGTATATAGATCTGTCATAGAAGGGTT
TAATTATATCTGCCTAATGATTTTCTCTCCTTAATGCCTCTATAGCTGTC
[C/T]
CTTAGCGAAGAATAAAACTGTGGACTGACCCCCACCCATTTGCGAAGAA
AGTACTGGGTCTTCAGCTTTCATTGTTCAGCCGGTGGTCTTTGTGGACAA
CACCAGGGGCCCCTCCTGCTGAAGACTGTCTGGACTTCCTGATTTTCACT
CCTGTATAAACCCCCTCCCCCATTACTACCTTTGTCTCCTTGGGGAAAAA
>rs4784220
AGAGAATTACTAATATTTTCTTCTCCAACCTTAATGCTCACTCCCTGAGC
TATGTGGATTATACTAGTGAAAGTACTGCCTTAACCCACTAATGTAAACA
GATTCCTTTTAAGAAGCCTCCCTATCGAGAATGGTCTGTAAGAGACAACC
ATGTCAACAAGTAAAGTTCCTTCTGCCTTCCCTCCTGGGGTGGGCTAAGA
[C/T]
GGACCCTTCTCACCATTCCCAATTACCTGCAGGAAGGCAGCAACAGAAT
AGAGAAGGAGGGTAACACAGTTTCCTGTACTTTAAATACCTCCCTGCCTC
CGCAAGTAAAACTTGAGCCATGCCAATGAGGGAAAAAGTGGCCGCGATTT
CCCAGGTGGCTTAGTAATACACAGTTTTCTGACACACTGCAACTCCATTC
>rs12598982
CACTACCAAATAATATTGCTTCTTGCATTAAGTATAGCATTGGTTTGCTA
GAATAATCCTAACAATGACAATGCAATGTACTTTAAATTGGGGTCAAAAA
ACTTTTTTCTTAAAGTACCAGATAGTCAAGCTTTTAAGCTCTATGGGTCA
TCCACAGACACACTGCAACTATACAAACTATATCACCCCTGTTGCAACTA
[C/T]
ACGAAAAGCAGGAAAGCAACCACAGAAGACAGACAATACATAAACAAAT
GAGTATGACTGTGTTCCAATAACATTTTATTAATAAAAACAGGCAACTGG
CCCATGGGTGGTAATTTGCTGAAACTTGGTTTAAATTATTATTAGTATCT
ACCTTTTTTTAAAAGATGATGTTTTTTAGGGTCTTTCCATCATGAAAATT
>rs4784222
TAATGATACTATAAAAACACCCAGCATAACGCCAGCATAAGGATGTGAAT
GAATGAGCAAATGAAAGGAGGGAGGGCAATGGCAAAATTTTACCTTGGTT
TCCAAGCTAAAGTATAACCTGAGTTTTTTAATGCTTTACAAAAAGGAAAG
GCAATCAGAACCATAATTAATATTTAAGCTGTACTGACAGGTTTTCCAGG
[C/G]
AAAATTCTATAGAACTTGTTATTCTCCAACTTCCAAAAATAAGCAATAA
ATTTTTTTTAAAGAGCTGTTATTTTAAGACTTTATTTGAACCAAAGAATG
TGTTTATGGTAACAACATACATGTTGTAAATTAATCAGTTTCAATGTTAT
AAAATAATACTTCCAAGAATAAATGTATCATATAATCTATGTACCTCATG
>rs17271951
TCATTGTTTGTAAAATCCAATTGTCATACTCTGGTATTTGTTCTCCAAAA
GAATCATCACTTTCATATTCACTACAGCATGAGATGAGATATTTAATAAA
TAATAGGACATCATAATCCAGGGATTAAGTAGTTTCTTTTAGCAAAATTT
AATATGAGGGTAGCATATGCCAGCCGTTCCTAGTTGAGCAACAATTTCTG
[C/T]
GTTGGGAGAGGATAACAAAGCCAAGTATTTTCCCATAAGAAGTTCACAC
TCTAAAAGGCCAGGCGCGGTGGCTCACACCTATAATCCCAGCACTTTGGG
TGGCTGAGGCAGGCGGATCACTAGGTCAGGAGATCGAGACCATCCTGGCT
AAGACGGTGAAACCCCGTCTCTACTAAAAATACAAAAAAATTAGCCAGGC
>rs9933638
GGAATAATTACAGATGATGCTAGGCAATATCACTAGATTGGGTTTTCATG
AAGCTCGTGACTGGATTTTGCCACAAAAATCACTAACTGCTTTGGCTTCC
AGAGAATGGCCTCATGGATAAGCCCCAAGTTCCAAAAACTACAAAATGTC
AAAATCACATTCAACTAATTAATAATTTTATGTCACAATAAGAATTAGGA
[A/G]
TCTCTAGAAGGCTGGGTACAGTAGCTCATGTCTGTGATCCCAGCACTTT
GGGAGATCGAGGCAGGCAGATCACTTAAGCCCAGCAGTTCAAGACCAGCC
TGGGCAACATAGGGAGACCCCGTCCTACAAAAAATATAAAAATCAGCTGG
GCATGGTGGCACATGCCTGTAGTCCCAGCTACCCAGGAGGCTGAGGTGGG
>rs9302556
AACACATAAGCCATTTTTTCCAAAGCTAGACAGCTTTTCTCTATCCACGT
TCTTTCAAATATTTTACCTAAGACAGAGAATCTCATATTAAATTCAATTT
CACACTTGCCAATCACACACGCCCACTCACAATTCCTCCAGATGATGCAA
TGTGGCCTAATGTGTATGCTTATGTATGTACATGTGCACACACAAACAGG
[A/C]
ATAATGTTTAAATACATATAAACACATTCATTGCTAGGTATTGGTTAAA
ACAAACAGGCTTACAGCATACCAACTTATTAAACATAATGATTGATATTA
GTAGTTTCATTAATGTAATTAACCTTTAGATTTGAATTATCAAGTTCAAA
GTCTAAGACCGAGAGCAGTGGTTCTCAGGCCGGTACAATAATGGCAAGTC
>rs7190749
CAGTCCTGCCCCTACCAGAAACCAGAGATATTTGCTGCTGGTAATAATGA
GGAGGGGGTTTCTAAGTACAAGATCAGCTCTATTCTTTCATTCCCTGGAA
ATCTGCCCTCGCCTCTGATACCTCAAAAAATATCTGTTGCATTTCCAAGG
GAAAGAACAATGATATAAGCCTTGCTGTTAAAAAAAAAAAAAAAAATGCC
[A/G]
CTACTTACAAGATAAAAGGCAAGGTGTTTTGTTTTTTTCCCCTTTCTCT
TAGACTAGGCATGAATAATCTCAAAATATTTGTTTGTCAATAAAGTGATT
AAAATAAAGTGATAGACAAGACATAATGATTAGAGAAATGAATCAAAGTA
ATATTTGGGACCAGGGTCTTACAAATCTAGAGTAAGTATGTTGAGCAGAA
>rs12443621
AGATCAAGATGTCTAGCATAATTTGAAAGTATGTTTCTATTTGACAGAAA
CCTTGGCTTGGAAAAGTTAAACTCTAAATATTTGTACAGAAAGAAGTATT
CTGGGTTTCATTCATTAAAAGTCATTGACCACTGCAGAAAAGGGAGAGCA
AGAGATTTACATATATTTTTCTTGACGTTTTATATGCATTAGGCCTGGCA
[A/G]
TGAACTTGAGGTAGGTATTACTATCTCCTTATTTCTAAGGGAATCAGAA
TACTGTAAACAAAGACATGGAGCTAAATAATGGAGTCAAATCCTGGCTTA
CTAGGCTCCACATCCATGTTGCTCCACTTATCAGACGGCCACCCAAACAT
ACAAGTTTAACAAATTCCAAATTATTGGGGCCTAAATAGACACTTTTTTG
>rs9933556
CATGGAGCTAAATAATGGAGTCAAATCCTGGCTTACTAGGCTCCACATCC
ATGTTGCTCCACTTATCAGACGGCCACCCAAACATACAAGTTTAACAAAT
TCCAAATTATTGGGGCCTAAATAGACACTTTTTTGTTTCAATATTGCCCT
TCTTCCCTTGTATTCTGAATATCATGTCAACCATGAATCATAAGCCAATG
[C/T]
TTACAAATTTAACTTCACATTATGAGTTTTGAGCCCAACTGATGCCTAA
CCACTAAACCATTCATATAACACACTGAAAAGAAGCCATTTAAGAAATGA
GAAAAATAGGTATTGTTTTCTCTGTATGGCTGTAAAAGCTGAGATAAGAA
CATTTTGGCAAGTTCAATAGAGCCGAAAGGCTTGTGAAGGCTATGGGAGG
>rs1362546
TCCACTATTTGCACTAGCATTAATCAACATCCAAAAAGTGTACAAATTAT
TTTATCTTTATAATCAATCTTCTGAAATGCTAACTTGAAAATATAAACAG
CACAATTACTTTTTTCTCTTTTCTTTCCCATTTGAAATGTGTGCTTGTGT
GTGTGTGTGTGTGTGTATCTGAATCTGTACATATAAATCAAATACACACA
[C/T]
ATATACTTGTTTTAAAAAAATCCTCAAAGAATCAATCACATATTACAGA
ACTGAAGGAATATTGGAGTTCATCTGGTCCAAAGTGTTGGTATTGTAAAA
TCACTTTCAAATTTCACAGTTATCCAAATGGTAATGAATATTAATACCTA
AGAAAATCAATCAAAGATTAACATTAAGACATTAGATTTTAAAAATCATT
>rs1075367
TTTTTAGCTACAAGGATATGTTTTTAAACCATATTGAATTACATAGAAAG
CATTTCCATTTTCCTTTCAGGCTTTCTTATTACATCAGTGAGAAAATCTC
AGTTTGGTGCGAATATGTCTTTAACACCTAATACTTGCTAATCTCTTTTT
TAGCAGAGAGATTGAGACACATTATAGGGGAAGAGAGAGAGAGGGAGAGA
[C/T]
GGAGGAGATCATACAGGACCCAGTTACAACCCTTCTGTAGCTTTGGTTT
GCTAGAATTAAATAGATTATTTTGTATTTATTGTATTTATTTTGATAGTT
ATGTGCTATTTATAGAAAGGAATACCAGTTTTCCATGTATGAAATAAAGT
GTCCTTTTGTTAACAAAAAAGCATATTAATTTTTATGCAAATGCATGAAT
>rs8046979
CAGCTTCAGTGGATGCAAGTATGATCTCTCCGATGATCCTGGGTCCCTTC
AGAGGCCTTTGGATGGTTCTGAGCAGTAGAACTCAAACTGTGATCCCAGG
AAGGCAGCATCAGCATCACCTAGCAACTTGTTAGAAATGCAAATTCTTGG
GCCCTACCCCAGACCTACTGAATCAGAAACTCTGGGTGTGGAGCGGCAAC
[A/G]
TGGATTCTAACAAGGCGTTCAGGTGACTCTCATGCTCAGTGAAGTTTGA
AAAGCCTGTGGGTAACGAGCACAGTCTCTGGAACCTGCCTGTTGATTTGA
AGGAATCCCAGATCAATCACCTCCTCACCTGAGACTTTGAACAAGTTATT
TAATTTCAGCCATGCTTCCATTTCCGTTTCTGTTAACTGGGACATTAATA
>rs1420529
ATGAACTCTTCAGTGCCCTCCCCTTCCCACAGAGGGCTGAAAGGGACTCA
CTCTTGTTACAAGAACACGGCCACTCCTCCCTCTCTTCCTCTTCCCAGTC
CATCCCTCACCCCAGTTTCAGCCACCATCCAAAAAGAAATCGCAGCATGG
CCCTTGAAGCACATTGAGCTTAAAAGTGCCTAGAAGATAAAGCCTAAACT
[G/T]
CAGCACGACAGGCAAGACCTTTCAAAATCTGGCCTCGACCATCCTCTCT
GGGTCTCCTTTCTGAGAACCCCTATTTTCTGCCTTTTCACAACTCCACAC
TTATGCATGATGCTGTTTTCCCTCCCTGGCTCACCCACCCCAGTGTTCTT
CTTCTGTGATGGTAACCTCCCCAGAAAGTCTCTTGAGCTAGAGTGATCAT
>rs11642645
CTCGACCATCCTCTCTGGGTCTCCTTTCTGAGAACCCCTATTTTCTGCCT
TTTCACAACTCCACACTTATGCATGATGCTGTTTTCCCTCCCTGGCTCAC
CCACCCCAGTGTTCTTCTTCTGTGATGGTAACCTCCCCAGAAAGTCTCTT
GAGCTAGAGTGATCATTATTAAATTAAAAAAAAAAAAATCCTACTTTGTT
[A/C]
TATTCTGATTTCAAAAACCTGAAAGGCAAGATATTTGAGGGAAAAAAGA
GAATATTGTTTTGTTGCTTTTTAAAAAATAAATTTGCAGGTCCTGACAAT
CCCGTGCTTTGTGTGTGCTGATCACACTGCAATCTAATCAGGTACAGTAA
ATAAAATGACTTGAGAACCTTTCTGACGAATGCAAAGGCTGATGATATTT
>rs1420533
TGTGATTTTTAAAAGGTAAGTAACAGGTGAATCAAAGCAAGGAAAAAGTA
AATTTGAGAAAATAATAAAGCCTAGATGGCCAGCACACAGGAATGCATAT
CCTACTGCCCTAGACAGCTACTAAATGGGTTTCCTGTTTGACTCCAAGCT
TCTTGGCCATCAATGCAAAGAAAGAACAAGAATGGTAAGATTAATGCCCA
[A/G]
TAAGATACAGCGAAGTGGATTTTTTTCAGTTCTTCTTGATATAGTAACC
TAAGAAGATTCTCTGTAAATCATCATTTAAAAAGACACCATGAGGTGTAA
TGAGCTATACATTGTAAATAGAACTTTTTAAAAAAAATGATGTTCACGAA
ACTATTACTCATAATCCTCTCAATACAAAGTGAAGCCTTAATCAGTGTGC
>rs1362548
TTTTAAAAAAAATGATGTTCACGAAACTATTACTCATAATCCTCTCAATA
CAAAGTGAAGCCTTAATCAGTGTGCAATTCAATAAAGCAGAGAGGGCCTG
TGAAGGCAACTGTGAAGTGGGGTGCCACATAATATGCCATCGTGAGCATT
CAGCTCTCTCACAAGTCTGGCAGAAATCTCTTATCAAATTAGGTTATCTG
[C/G]
ATGAACGGAAGGAAGCAAGGCATCTTCTTCATGCCATTCTCCTTAGATA
TATTATTTCTTCCAACAGATTTTATTAACACAAACTGGGCAGCAGGGACT
TCAAGAAGGACAAAAATCCTGTGTCAAAACCATTACATATGTTGGCAACA
TTTATCATCAGGAAAAGCGTCCCAGGAAGTTGAATTCTAATGTATGTGTG
>rs2193094
CTGGGCCTGGACTCAAGGGTTCTTGTGTACTTTCCTGCTGTCCGAAATAC
TGCCATCATGAACAAGCCCAGGCTGGCGTGCTGGAGGTGAAAGATACATG
CCAGAGAACTGAGGGACCCCGGTGGACAGCCAGCCAACCTCCTGAAGCAA
ACCCTCCCTGCCAATCTGCAGTTGACTGCAAACACATAAGAGACCCAGAA
[G/T]
AGGATAAGCACTGCCCTTCTGAGCCCAACCTAAATTGCTGACATGCAGA
ATCAGAAACAAAATATGTGTTTTTGAAGTCACAAAGTTTAGAGGCGGTTT
ATCACTCGCGATAGATAACTGGTACAGACAACACGGGTCTGCCTAGAAAG
TGAAAAACATACGAAGGCAAAACAGTAAGAGGTCAAGCTTTAGGAGGATA
>rs4783780
TCTCATATCCATAGAATATCCAGGTGACCTATAGGTTTCTTGAAGGCAGA
CCACATCCAATCTCAGCCTCAACAAGAAGGGCGTTCCTAATATAAACAAT
ATTTATGATATTTACATTACATAAGCACAGGCTCATTTTGGTTTCCACAA
AGAATAATAATAAACATAGACAATGGAAATCCTCAGAAATCTTCAAAATC
[A/C]
TCAAGCTTCACTCCAGTTAGCCGTTTCAATGGACTTTGCTTTCCTATCC
TACACCCACATTCTCTCATTGTGACATCAGCACTTTGTTTTTCCTCCGAA
GAACCACTGCTCCATACTCTTAGCTCCTGTGGCTTCCAAGAAGATGACAG
TGTCCAGCTCCCCCACTGGCATGCCTACGTGGCTACGAGCCTGGGCATGG
>rs3112581
TTACATTCTCACAGCTCAGCCTCTTCAAGAGGGAGAGATTCTTTCTTGCA
TGTTTCAGCAGCTTTTCTGGATCAAGTCTCATTGGATAGACTTGGGTCCC
ATGCCCAGGCTCGTAGCCACGTAGGCATGCCAGTGGGGGAGCTGGACACT
GTCATCTTCTTGGAAGCCACAGGAGCTAAGAGTATGGAGCAGTGGTTCTT
[C/T]
GGAGGAAAAACAAAGTGCTGATGTCACAATGAGAGAATGTGGGTGTAGG
ATAGGAAAGCAAAGTCCATTGAAACGGCTAACTGGAGTGAAGCTTGATGA
TTTTGAAGATTTCTGAGGATTTCCATTGTCTATGTTTATTATTATTCTTT
GTGGAAACCAAAATGAGCCTGTGCTTATGTAATGTAAATATCATAAATAT
>rs3112580
TTTTTTTTTTTTTTTATCATATCAGCCAGGCTGTACTCAAGTAGTGGGAA
GTTGTCCAGCAGCAGCTCCAAGTTACATTCTCACAGCTCAGCCTCTTCAA
GAGGGAGAGATTCTTTCTTGCATGTTTCAGCAGCTTTTCTGGATCAAGTC
TCATTGGATAGACTTGGGTCCCATGCCCAGGCTCGTAGCCACGTAGGCAT
[A/G]
CCAGTGGGGGAGCTGGACACTGTCATCTTCTTGGAAGCCACAGGAGCTA
AGAGTATGGAGCAGTGGTTCTTCGGAGGAAAAACAAAGTGCTGATGTCAC
AATGAGAGAATGTGGGTGTAGGATAGGAAAGCAAAGTCCATTGAAACGGC
TAACTGGAGTGAAGCTTGATGATTTTGAAGATTTCTGAGGATTTCCATTG
>rs9931232
TTGAATTAAGATTAAAGATAGAATGTTAATCATACAAGAAGGGCCATTTT
CCAGGGCCTGGCATGTTTTACACAAGCATGAAACATATTTAAGAGAACAT
GGCCTCCCTGAGGAAGAGATTAAGGATACAAACCTAGGAAACAAAGAAAA
TAAAACGATCTACCTCAAAGGGATAAGATTAACCTGTGAGAAGCTCATGC
[A/G]
GACCTTAAATAAAAGCCTCTACTCCTTATATTTTTAAACAGGATTTTTC
TTTTTTTTTTAGAGCACTAATTCCACCTTCAAAATATATTTGAGTTTGTA
AGTATACTCAACAAAAATCCTGTCCTTTCTTATTAACCATACTCAAAACA
GATTCTCTAAAATAGTTATGACACCATTAACATTTCATGAAATCCAAAAC
>rs1123428
CCTGGCTATATTTTATTTGAAGTTATTCTCCTACTTGCAACGACTTTTGA
ACCCACAAAACTCTTTCATCCATTCTCCACAGACACAGTACACTTTCATG
AGCACTGACATTTGAAAAGGATCCATATACTCACAGAATGTAAAGATAAT
ACACATGCATATAATTGATACAAAGGACTCTGGTCATTTGTTAAAGAGAG
[A/T]
AATGAATGAAACCCTATTATTCACGGTTTGTAAATAAATTTAAAGATAA
GCGCTAACTTTCACTTGTTAAGCCTAGAGCCAAGAGTTCGGTGTTTCTTC
TAAAGTTTCCCAGATGGTCATTAAAACAAGAGAGGGAAGAAACATACTTT
CTTTGAAATCCATCATTTCCCATCCAGAAAGATAATACATGTCATCAAAA
>rs3095604
TCATTCACTGCTCTGGGAGAGAAAATGTCCCTATTTTCCTAAAAGGTCCT
AGCTGATTAACTTCCCCACTCTACACTCACCCACCCCTCTTTATTTCTTT
ATCTTTCTTAAGCTCCATCTTTTTATTTAATTTTTTTTCTCATCGGCTTT
ATTGCTTTGAGTTAATCTGAGAGTTTGGCCTTCAGCCTTGGCGAAGGGAA
[C/G]
GGGCTTTATACTAAATGGGAACGTTGTGGAGAGGCCGTGAGATCTGCGC
CCTCCCCAGTTTAGTTATCTGCCAGCTACTTTAACCAGAGTGGCGACATT
CTGAAATATTTCCTATGACACTCGCTAACAGTTTCATCCCCTGGTCACAC
CTTGCTGGCCCAGGGCCTGAGCTTTTTCAGACAAGCATGACATGGGTTTT
>rs4784227
GAGTTGATCAGTAAATATTTGCTGAATGAAAGAATACATGAATGAAAAGT
CAGAGCCCTATAGGTCAGCATGGACGGCGGTAAAGGAACCTGGCTGAGCC
TGAAAGAGAATGTGATCTAAGATTAAATCCAGGATATGCTGGTAAATGTT
TAACAGCCAACTCTTTGGGGAGGAAAAAAGTCCCAATTTGTAGTGTTTGC
[C/T]
GATTATTGTGATGTAAATACTCCCATCATGACCAATTTCAAGCTACCAA
CATGCTGACACTGAACTTGGAGTTGGAAGGAGATGAACAGGCATAATCAG
GTCTCGTGAGATGGCCCAAGCCGGCCCCAGCACTCCACTGTTATATATGA
GGCTAGAATTACTACATAACTGGAATAGCAACTTTCTGGACCATATGCCT
>rs12922061
ACTTCATTTTGAGGAAAAGAACAGTTACTACCATTCATTCATTCACTCAT
CACACCAATATTACTGAATCCTACCCTGGACCAGCATTGTTATGGTAGAT
CTTGATAATATAGTGACAGACAATACCATCCTTCCCACCATTTACATAGT
AGACTGCCTGGGGCTCATAGTCTACTCACATCTCAGCACATGAGATAGTC
[C/T]
CATTATCACATTCTCTTCTATCTCTCTCACAGGCACTCACTCACCAGCC
ATGGAGAACTTAATGGGCATTAGGTTCTTAATCCTGCTGGGTATAATTGG
AGATAACCACCCTGGCTGACTGAGCACTATACCAGGCTACTCATGGATGA
TTTGGTTATTAGACGTAATCAGAGTGGCCCTTGTATGCTAAAAAGGAGAC

TABLE 29
Multi-Ethnic cohorts:

>rs4784227
GTAAATATTTGCTGAATGAAAGAATACATGAATGAAAAGTCAGAGCCCTA
TAGGTCAGCATGGACGGCGGTAAAGGAACCTGGCTGAGCCTGAAAGAGAA
TGTGATCTAAGATTAAATCCAGGATATGCTGGTAAATGTTTAACAGCCAA
CTCTTTGGGGAGGAAAAAAGTCCCAATTTGTAGTGTTTGC
[C/T]
GATTATTGTGATGTAAATACTCCCATCATGACCAATTTCAAGCTACCAA
CATGCTGACACTGAACTTGGAGTTGGAAGGAGATGAACAGGCATAATCAG
GTCTCGTGAGATGGCCCAAGCCGGCCCCAGCACTCCACTGTTATATATGA
GGCTAGAATTACTACATAACTGGAATAGCAACTTTCTGGAC
>rs17271951
TAAAATCCAATTGTCATACTCTGGTATTTGTTCTCCAAAAGAATCATCAC
TTTCATATTCACTACAGCATGAGATGAGATATTTAATAAATAATAGGACA
TCATAATCCAGGGATTAAGTAGTTTCTTTTAGCAAAATTTAATATGAGGG
TAGCATATGCCAGCCGTTCCTAGTTGAGCAACAATTTCTG
[C/T]
GTTGGGAGAGGATAACAAAGCCAAGTATTTTCCCATAAGAAGTTCACAC
TCTAAAAGGCCAGGCGCGGTGGCTCACACCTATAATCCCAGCACTTTGGG
TGGCTGAGGCAGGCGGATCACTAGGTCAGGAGATCGAGACCATCCTGGCT
AAGACGGTGAAACCCCGTCTCTACTAAAAATACAAAAAAAT

1. A method for determining a susceptibility to breast cancer in a human individual, comprising determining the presence or absence of at least one allele of at least one polymorphic marker in a nucleic acid sample obtained from the individual or in a genotype dataset derived from the individual, wherein the at least one polymorphic marker is selected from the polymorphic markers set forth in any one of Table 10, Table 15 and Table 19, and markers in linkage disequilibrium therewith, and wherein the presence of the at least one allele is indicative of a susceptibility to breast cancer for the individual.

2. The method according to claim 1, wherein the at least one polymorphic marker is located within a genomic segment with the sequence as set forth in SEQ ID NO:4, SEQ ID NO: 5 or SEQ ID NO:6.

3. The method according to claim 1, wherein the at least one polymorphic marker is selected from the markers set forth in any one of Table 10, Table 15 and Table 19.

4. The method according to claim 1, wherein the at least one polymorphic marker is selected from rs4848543 (SEQ ID NO: 1), rs3803662 (SEQ ID NO: 3) and rs13387042 (SEQ ID NO:2).

5. The method according to claim 1, further comprising assessing the frequency of at least one haplotype in the individual.

6. The method of claim 1, wherein the susceptibility conferred by the presence of the at least one allele or haplotype is increased susceptibility.

7. The method according to claim 6, wherein the presence of allele A in marker rs4848543, allele T in marker rs3803662 and/or allele A in marker rs13387042 is indicative of increased susceptibility to breast cancer.

8. The method according to claim 6, wherein the presence of the at least one allele or haplotype is indicative of increased susceptibility to breast cancer with a relative risk (RR) or odds ratio (OR) of at least 1.20.

9. The method according to claim 6, wherein the presence of the at least one allele or haplotype is indicative of increased susceptibility with a relative risk (RR) or odds ratio (OR) of at least 1.25.

10. The method according to claim 1, wherein the susceptibility conferred by the presence of the at least one allele or haplotype is decreased susceptibility.

11. The method of claim 1, further comprising analyzing a sample comprising genomic DNA from a human individual or a genotype dataset derived from a human individual for the presence or absence of at least one at-risk allele of at least one at-risk variant for breast cancer not in linkage disequilibrium with any one of the markers set forth in Table 10, Table 15 and Table 19.

12. The method of claim 1, comprising determining the presence or absence of at least one allele in at least two polymorphic markers, wherein the presence of the at least one allele in the at least two polymorphic markers is indicative of an increased susceptibility to breast cancer.

13. The method according to claim 1, further comprising a step of determining the presence or absence of at least one high penetrant genetic factor for breast cancer in a nucleic acid sample obtained from the individual or in a genotype dataset derived from the individual.

14. The method according to claim 13, wherein the high penetrant genetic factor is BRCA2 999del5.

15. The method according to claim 1, further comprising assessing the individual for estrogen receptor or progesterone receptor status.

16. The method according to claim 15, wherein estrogen receptor positive status or progesterone receptor positive status in the individual is associated with increased risk of breast cancer.

17. The method according to claim 16, wherein the increased risk of breast cancer is associated with rs13387042 allele A and/or rs3803662 allele T, and markers in linkage disequilibrium therewith.

18. The method of claim 1, further comprising analyzing non-genetic information to make risk assessment, diagnosis, or prognosis of the individual.

19. The method of claim 18, wherein the non-genetic information is selected from age, gender, ethnicity, socioeconomic status, previous disease diagnosis, medical history of subject, family history of breast cancer, biochemical measurements, and clinical measurements.

20. The method of claim 1, further comprising calculating combined risk.

21. A method of determining risk of developing at least a second primary tumor in an individual previously diagnosed with breast cancer, the method comprising determining the presence or absence of at least one allele of at least one polymorphic marker in a nucleic acid sample obtained from the individual, or in a genotype dataset derived from the individual, wherein the at least one polymorphic marker is selected from the polymorphic markers set forth in any one of Table 10, Table 15 and Table 19, and markers in linkage disequilibrium therewith,
and wherein the presence of the at least one allele is indicative of risk of developing at least a second primary tumor.

22. The method according to claim 20, wherein the at least one polymorphic marker is rs4848543 (SEQ ID NO: 1), and markers in linkage disequilibrium therewith.

23. A kit for assessing susceptibility to breast cancer in a human individual, the kit comprising reagents for selectively detecting at least one allele of at least one polymorphic marker in the genome of the individual, wherein the polymorphic marker is selected from the markers set forth in Tables 10, 15 and 19, and markers in linkage disequilibrium therewith, and wherein the presence of the at least one allele is indicative of a susceptibility to breast cancer.

24.-28. (canceled)

29. An apparatus for determining a genetic indicator for breast cancer in a human individual, comprising:
a computer readable memory; and
a routine stored on the computer readable memory;
wherein the routine is adapted to be executed on a processor to analyze marker and/or haplotype information for at least one human individual with respect to at least one polymorphic marker selected from the markers set forth in Table 10, Table 15 and Table 19 and markers in linkage disequilibrium therewith, and generate an output based on the marker or haplotype information, wherein the output comprises an individual risk measure of
the at least one marker or haplotype as a genetic indicator of breast cancer for the human individual.

30. The apparatus of claim 29, wherein the routine further comprises a risk measure for breast cancer associated with the at least one marker allele and/or haplotype, wherein the risk measure is based on a comparison of the frequency of at least one allele of at least one polymorphic marker and/or haplotype in a plurality of individuals diagnosed with breast cancer and an indicator of the frequency of the at least one allele of at least one polymorphic marker and/or haplotype in a plurality of reference individuals, and wherein the individual risk for the human individual is based on a comparison of the carrier status of the individual for the at least one marker allele and/or haplotype and the risk measure for the at least one marker allele and/or haplotype.

31. The apparatus according to claim 29, wherein the at least one polymorphic marker is selected from rs4848543 (SEQ ID NO: 1), rs3803662 (SEQ ID NO: 3) or rs13387042 (SEQ ID NO: 2), and markers in linkage disequilibrium therewith.

32. (canceled)

33. (canceled)

34. A method of identification of a marker for use in assessing susceptibility to breast cancer, the method comprising:
d. identifying at least one polymorphic marker in linkage disequilibrium with at least one of the markers within the genomic segments with sequence as set forth in SEQ ID NO:4, SEQ ID NO: 5 and SEQ ID NO:6;
e. determining the genotype status of a sample of individuals diagnosed with, or having a susceptibility to, breast cancer; and
f. determining the genotype status of a sample of control individuals;
wherein a significant difference in frequency of at least one allele in at least one polymorphism in individuals diagnosed with, or having a susceptibility to, breast cancer, as
compared with the frequency of the at least one allele in the control sample is indicative of the at least one polymorphism being useful for assessing susceptibility to breast cancer.

35. The method according to claim 34, wherein an increase in frequency of the at least one allele in the at least one polymorphism in individuals diagnosed with, or having a susceptibility to, breast cancer, as compared with the frequency of the at least one allele in the control sample is indicative of the at least one polymorphism being useful for assessing increased susceptibility to breast cancer.

36. The method according to claim 34, wherein a decrease in frequency of the at least one allele in the at least one polymorphism in individuals diagnosed with, or having a susceptibility to, breast cancer, as compared with the frequency of the at least one allele in the control sample is indicative of the at least one polymorphism being useful for assessing decreased susceptibility to, or protection against, breast cancer.

37. The method according to claim 34, wherein the at least one marker within SEQ ID NO:4 is selected from the markers set forth in Table 20.

38. The method according to claim 34, wherein the at least one marker within SEQ ID NO:5 is selected from the markers set forth in Table 21.

39. The method according to claim 34, wherein the at least one marker within SEQ ID NO:6 is selected from the markers set forth in Table 22.

40. The method according to claim 34, wherein the at least one polymorphic marker in linkage disequilibrium with at least one of the markers within the genomic segments with sequence as set forth in SEQ ID NO:4, SEQ ID NO:5 and SEQ ID NO:6 is selected from rs4848543 (SEQ ID NO: 1), rs3803662 (SEQ ID NO:3) and rs13387042 (SEQ ID NO:2).

41. A method of genotyping a nucleic acid sample obtained from a human individual at risk for, or diagnosed with, breast cancer, comprising determining the presence or absence of at least one allele of at least one polymorphic marker in the sample, wherein the at least one marker is selected from the group consisting of the markers set forth in Table 10, Table 15 and Table 19, and markers in linkage disequilibrium therewith, and wherein the presence or absence of the at least one allele of the at least one polymorphic marker is indicative of a susceptibility of breast cancer.

42. The method of claim 41, wherein the at least one marker is selected from rs4848543 (SEQ ID NO: 1), rs3803662 (SEQ ID NO:3) and rs13387042 (SEQ ID NO:2).

43. The method according to claim 41, wherein genotyping comprises amplifying a segment of a nucleic acid that comprises the at least one polymorphic marker by Polymerase Chain Reaction (PCR), using a nucleotide primer pair flanking the at least one polymorphic marker.

44. The method according to claim 41, wherein genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis.

45. The method according to claim 44, wherein the process comprises allele-specific probe hybridization.

46. The method according to claim 45, wherein the process comprises DNA sequencing.

47. The method according to claim 41, comprising:
3) contacting copies of the nucleic acid with a detection oligonucleotide probe and an enhancer oligonucleotide probe under conditions for specific hybridization of the oligonucleotide probe with the nucleic acid;
wherein
a) the detection oligonucleotide probe is from 5-100 nucleotides in length and specifically hybridizes to a first segment of a nucleic acid whose nucleotide sequence is given by SEQ ID NO:4, SEQ ID NO:5 or SEQ ID NO:6;
b) the detection oligonucleotide probe comprises a detectable label at its 3′ terminus and a quenching moiety at its 5′ terminus;
c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
d) a single base gap exists between the first segment and the second segment, such that when the oligonucleotide probe and the enhancer oligonucleotide probe are both hybridized to the nucleic acid, a single base gap exists between the oligonucleotides;
4) treating the nucleic acid with an endonuclease that will cleave the detectable label from the 3′ terminus of the detection probe to release free detectable label when the detection probe is hybridized to the nucleic acid; and
5) measuring free detectable label, wherein the presence of the free detectable label indicates that the detection probe specifically hybridizes to the first segment of the nucleic acid, and indicates the sequence of the polymorphic site as the complement of the detection probe.

48. (canceled)

49. A method of predicting prognosis of an individual diagnosed with breast cancer, the method comprising determining the presence or absence of at least one allele of at least one polymorphic marker in a nucleic acid sample obtained from the individual, wherein the at least one polymorphic marker is selected from the group consisting of the polymorphic markers set forth in Table 10, Table 15 and Table 19, and markers in linkage disequilibrium therewith, wherein the presence of the at least one allele is indicative of a worse prognosis of the breast cancer in the individual.

50. (canceled)

51. (canceled)

52. (canceled)

53. A computer-readable medium on which is stored:
d. an identifier for at least one polymorphic marker;
e. an indicator of the frequency of at least one allele of said at least one polymorphic marker in a plurality of individuals diagnosed with breast cancer; and
f. an indicator of the frequency of the least one allele of said at least one polymorphic markers in a plurality of reference individuals;
wherein the at least one polymorphic marker is selected from the polymorphic markers set forth in Table 10, Table 15 and Table 19, and polymorphic markers in linkage disequilibrium therewith.

54. The medium according to claim 53, wherein the polymorphic marker is selected from rs4848543 (SEQ ID NO: 1), rs3803662 (SEQ ID NO:3) and rs13387042 (SEQ ID NO:2), and markers in linkage disequilibrium therewith.

55. The medium according to claim 53, further comprising information about the ancestry of the plurality of individuals.

56. The medium according to claim 53, wherein linkage disequilibrium is characterized by numerical values of r² of at least 0.2 and/or values of |D′| of at least 0.8.


SEQUENCE LISTING (6 sequences)

SEQ ID NO: 1 - DNA, 600 bp, Homo sapiens
caggataagattatttctgaggctgcttccatcagtggttctgtgagctggcccatgtattcagccagtcagtaggccatcaagcatgcatctacgcaaggcactagaagggttacatacaatgggaaactgaggcccagaaggaggaaagatgaagtgcccaaagttaaggagataaaagcagaagtgcaggtaagagaaggagtattcatattactgcaggaatccaaattctacaagaacatacgcttgttctttaaagggagccctcgctttaagcattatttgccgatcaataccmattaataattacaggacatgggcaaatattctagcaaaggtcctgaggtcttcctagatgtgcccaaaataacaaggactccccccgcaggtggcaccaaaagtcggaggatgtgttggaagctgaccttccccacctgtccctgcctcacccacagtcacccacccctctaggatgtaatggtgaatcattcaacacatgggccaccttgggaagtcgcttaacctctctcccttttcacgttataaaatgggcatagtcattacacaaatgcataaatcaacctcaattttctcatc

SEQ ID NO: 2 - DNA, 600 bp, Homo sapiens
ctgacatagccgcctgtgcaggtttttgggaccaggtctgccatctgtattcccaggctgggctggagcacctgaggctactatggttacagaaaggaacctggcatccgaaggtataggctcaaatcccagctctcatgattgctagctttgaggtttcaggttcatttcttaagtgctctgagctttggtgccctcctcttcaaaatgggaatgaggagcatgtctagctccctgttttgttgcagtgaatagctacatgaagaacagctaaaccagaacagaaagaaggcaaatggargctacagaaaccaaggatttccttgttgaatcgaatcttccttcaatcttccttcaccacactagtggatctccctgtgggagggatgttgagagtgctccgtgttttttttgttttttttttttttttttttgtgatggagtctcactctgtcacccagactggaatgcagtggtgcgatctcggctcactgcaacctctgcctcccaggttcaagtgattctcctgcctcagcttcccaagtagccaggactacaggcatatgccatcacacccagctaatttttgtatttttagta

SEQ ID NO: 3 - DNA, 600 bp, Homo sapiens
gctagtccttggctgttctgtgatcaaatgcaattgtttcccatttcttaagaaacagaatctttattagactcagttcttttcttggttatttttacaattttattgttctatggttattaaaaaataacatgtcatataaattaagcctagaaacatggatgtttatcaaagaaaggattgtcatccaaagcaccaactatgagagatatctatgtgcaatggtatatagatctgtcatagaagggtttaattatatctgcctaatgattttctctccttaatgcctctatagctgtcycttagcgaagaataaaactgtggactgacccccacccatttgcgaagaaagtactgggtcttcagctttcattgttcagccggtggtctttgtggacaacaccaggggcccctcctgctgaagactgtctggacttcctgattttcactcctgtataaaccccctcccccattactacctttgtctccttggggaaaaaaaatgtttttcacttaagcaaaataataactacccagtatttgtttctggagtgaaattgtaaacttcattgtacattcttcctggctttgatgcttatc

SEQ ID NO: 4 - DNA, 94080 bp, Homo sapiens
ggttgacacaaacacgggaagattctgcaggaaacacaaggcagccacggcctctgtccctgagaggagcagtgagggagccgcagggctgggctgcggcgtgacactagggacgtggggggaatgcaagtgggtccagacctgggggccaccagtgcaggagcaagggcaggccggccatcagagctgggcggggaggggggcgctgagcttggcgtgggagtttccccaacggactgtgtcttccagggaccctcgcccctcatgcccctctgcgtcctgaagggaggaggcaccacccctctgtggcatctgctttttagggctgacgatggaacaagctcatggacaggagtgaagcccctggcacatagcaggtgccacagaagggtggtgaagtctgccctagatcagagatcattcagggggtctgttaacttggatgaagaagaaatacactcttatcactaacatcaggaaggtaagcaaggaaccacacatagaattgacaataagtgtgactttgccaccaagaagaatcacaaagttttattttattttattttattttattttactttaagttctgggatacatgtgcagaatgtgcaggtttgttacataggtatacttgtgccatggtggtttgctgcacccatcaacccgttatctaggttttaagccccgcacgcattaggtatttgtcctaatgctctccttcccctcgctccccacccccaaacaggccccggtgtgtgatgttcccctccctgtgtccatgtgttctcattgttcaactcccacttatgagtgagaacatgtggtgtttagttttctgttcctgagaatcatagagttttcatagcccagtacaataatcacagatacatgacatagagttgtgtgacatgtagaccccaatagtccggttaatgggatgctttagttaaaaagtacatagattactgtattataagtttatgttttaggatattttgatgattactttttagaaatagtttcctttgtaacctttattttacttaatgcatatgaaagaatcattcagaggaggggtccatggcttcaccaagatgaaacaggtcattggcatgaaaaaggttaaaacgctgtgccctggaaccaggagtttatgatccaggcctgcagcatgctgtatgaccttggccacataacttcattgtgcctcagttgcctcaactataaaatggggataataaacctgtgacatatgatgcttggggggttaaatgtgacaaaagtcttagcactctgtgtgccatacagtgagggccccagcatgggcattgatgcaccgtcttgattcctcctgcctttcccaggtacagggagcttccccagcctgcctgcatctgagaagattccaccagagaaacagaaccaataggagacatatattaagagttgtgttgcggccaggcctggtggcccatgcctgtaatccctttgggattacactttgggattacagcactttgggaggccaaagcgggcctatcacctgagttcaggagttcgagaccagcctgaccaacgtggagaaaccccgtccctactaaaaatacaaaattagccaggcgtggtggcaggcatctgtaatcccagctactcgggaggctgaggcaggagaatcacttgaacttgggaggcggaggttgcagtgagctgagatggtgccattgcactccagtctgggcaacaagagcaaaactccatctcaaaaaaaaaaaagagagagatgtgttgcaagaaatcggcttacgtgaccgtgggggctggatccatagcgcaggctctcaggaggggcagtccagaactctcaggctgatgctgcaggccacaggtgggatttcttctcaagaaagcctcagctctgctcctaaggcctttcaactgattgactcatgcccactcagattattgagggctatctcctttatttaaggttaaccggcccagtgcagtggctcacgcctataatcccagtattttggcaggaagattgcttgaggccaggagtttgagacaggactggacaacagagcaagagcctcatctctacaaaacataaaaaaattagcccggtgtggtggcacactcctatactcctagctactctggaggccaagccgggaggatcacttgagcccaaaagttcaaggctatactcctagctactctggaggccaagcggggaggatcacttgagcccaaaagttcaaggctgcagtcagctatgatcaagccacttatactccagcccagaaaacagagtgagaccctgtctctaaaacaacaacaacaaaaaaccccaaccaattgtagatatcaactacattgctgtggcttgaatgtctcctccaaaactcaatctgaaacttaatccctaatttaatagtattggcaggtgacagggcctttcagaggtagttgggtcatgtgggcattcatggattaatgggttaatagattaatgggttatcatgggagtagattagctatcacaagagtgagtctgttacaaaaatccagcttggctctctcttgtgagcccctccccatacgatgccctgcactgaccattagcaagaaagccttcatcagatggaccccctcaaccttggacatctcaatcttcagaactctaagaaataaatttcttttctttataaattgcccagtctctggtattcagttatagcaacagaaaatggactaagatacacatctacgagatacctccacagcaactcctagattagtgttgatggaataactggggattgtagcctagccaagtcgacacctaagaccggccctcatgtcacccaaggacaagacagcgaggtcctgaagagtttcatgaagaggaggggaaggagagagtggggagctcaatgtggacccctcaattaatttcccaagtgctcaatgaggcctgccccacctctcatccagctccttggccaggccctgcccccactcacctctcttatgtgggacacgtgagcccctccagtgtgccaggagcacatgggtacaggcacacatagagaatgccgtgtccacaggaaacccagtgagcctgaagggccttggcgtgccatttacaggtcactcacttgggccaggcctcactagggatgccagggtgaacaagacgtggccagtgctcttgaagggctcccacctggtggaggtagcagacaagtaaggggacaaaggcccctcagaagtacacatctggtgggccgggcgcggtggctcacatctgtaatcccagcactttgggaggccgaagcaggcatcacttgaggtcaggagttcgtgaccagcctggccaacaggtgaaacccagtctctactaaaaatacaaaaattagccagacgtggtggcgcgtggctgtaatcccagctactcgggaagctgaggcaggagaatcacttgaacccgggaggcggaatttgcagtgagccgagatcacgccattgcactccagcctggacacagagcgagactccatctcaaaaaactaaaaaaataaataataaaaagtacaaattaggtgtggttatcgcctggagagccaggagcatccttgggaggggacttggggaggggtccgctgggctgctctagcccactctgggccagaatcgtgattctaattgcaggtaggctttaccctatcaaagccccctgggctggggactctgtgaagaagcaagatgtgcttgaaacctccctctctgctgaaaaatctagacaagctcctgaccggctgcattgctgcttcagcccctcctcagggaagcgggaggccctgggtctctacgggtgctctgtgaaagactgcagaggctggtggggctgcattcctgtctgtctgcctaggacagtgcctactgacttctgactgctgccccatggtccagggctccaggtacaccactgcaatgtcactttgtgcttggggttccaactagacaaaagccccacgttgcacagcccttgagccgtcccgggtagcatgccccctggtggccgtgggcagttattaccgttaatcctttcttgcccagatgcctctagcgtaggccacagcagccggccgggttcgttgtgtctggaaagaccagagaagggttcccggagaaagaggccgtctgatcggagttttatgaaccagaggataaagaaaggaagggatttctgggcctcagaaaaccacatgccaacgcatgggggtattaaggataggggctcttgaacgcccaggctggtaaaatctagaacggcagagggaggaggggtaatttttctggttggtagaaataaagtggggatgaggaagagcacgaaagtctgtcggcaaacctgaaagtgggagcgagcttttgtcaaatccagggtcaacctgagctctcattaattgggaaactggagagtcagatcctctaaagaaagtggaaatatttgttctaaagaacccctgccaggtccccgtttacgtgactcataaagaggccttcaggcagaaaggatcccagtgcaccagtaacagaagggctccaggaccagagggaatggacagggaaccaccacccacaccaagcacaggcccattgtcggcatgaggagcctcgcccctgcagatcacctcccagaggggagcggatcagagattaaattccacacctttctacaaaatcatagcagtttgaaagtgccaaggggagtaggtgaggaggtttcgaggcatgtatgcagccgtttggagacaccagccatgtcgcctgagttttctgatgtaatcctcataacaaccattccagatttaagtgattagtcctgttttacagatggggaaactgaagctcagagaggttaagcaacttccccaaggtcatacagtagccaagtagcataagtggtattcgaacccagttctatttggagccttcacaggctttttgctgcattccacaagagtctgtatattcagaagcaagtcttccacagccatcataagtcatcctcacagccggctggcagggagggaacattgccactgtagctgggatcatagatatcgtggtctccatttgacggataagactgaggtctggaagagagtcagtcagtctgtgtgataagcctgaaccaccctctccttcagttcctcccctaacccaacatcctccgggtctggaaggacagtgtcttgagcaacttcttgcagctatacttctccagctaatagtctagaaaaccgagcctggtcccgagtctcctcctcggggtacgagccgagggcactgatgggggtcaggaggtgctggctaaaagacggtggtgcaggtgcccctggctttaagcgcgcccaggttataaaaagcttggttcccatgctggccctaattctccatgcactgagtaccttaggtcccttctctgggtctgttgctcccctgtaaaacaagggggtgggagaaatgaccttcaagctcaaacaccatatgtccacctgggttggtatagcagattgcattagtttgttaggactgttttaacaaatgccacagactgagtggcttaaacaacagacatttattctctcacagctctggaggctggaagtctgagatcaaggcagtggctggcagggttgtttctcctgaggcctctctccttgtagccacccctcttctccctgtgtcttcacatggttgtccctgtgtgtgtgtgtctgtgtcctaatcacctcttcttataaggacaccagtcacagaggattaccggccacccataggacttcattttaccttaattatctctttaaagtctccatctccaaattagtcacattctgaggtactgggagtgaggacttaaacatacgaatttggaggataggacacaattcagcctataacacagatagaagacactgctcattttaaaaaagaagtagcaggctgggcgcagtggcccatacctgtaatcgcagcactttgggaggccgaggcgggaggatcacctgaggtcaggagttcgagaccagcctggccaacatggtgaaaccccgcgtctactaaaaatacaaaaattagccgagcatggtggcatgctcctgtaatcccagctactcgggaggctgaggcaggagaatcacttgaacctgggaggtggaggttgcagtgagctgagatcgcactgctgcactccagcctgggtgacagagcaagactccgtctcaaaaaaaaaaaaaaaaaaagaagtagcagtcccagccctttaacagcgaacttctgatacatttaaaacagaatctgattctcactttttcttccaggcccacttcaatgccactaatgaggcctatctgtctgcaatctgatattagaggccaaaaataatctttctatttgccacttactgggcaccaatgtcctacctgacacatgctggctcaggtgggggaggaggtacggtccctggccctaggaacgctccatcttagaaagagacagaatgtgctggggtcagagtaaaaagtgcatgggcttcagcagcagatcccagcatggctaccaccagctgggggaccttgggcaagagccccctgtgagcctcagttttctcacctggtcaatgggactcacactactgcggcacacattgtgcgcccacattgtctcggtgcacaggggccacccttcaaactgccagggtctgcacctctgtgcccaaggtgcagaaagggctggctgccagaacccactctgcattttgcctctttgcacccgaggctggaagagccctggaatcactgactcgctggagcatccattcaccagtggccatgggagctgatgcataaatattccagcgctctggtccttcgagagggacactctgtgacatgcgttgctcacctgtgacatgcccctcccagcgtctcctgacctcccctcccaaatgcactacttgcacttgctctcattcccattctgcttgctcacaggctggatcacacaagaccacacatgcgtgtacactcgtgcctgcccatggcaagtattcaaaaactgcttgctattttgtgggtggtggtgaagggaatagtatatttataatacttcaggccaggaatatacatctcctctgttttctgacccttccaggagtagggtggattccacccgcttttttgtaagccctcagaagtgctccaggctctctgcacacccccagatctcccctgagcttcagccctcacccagctccccagtcaatttggcccctgtgacgatgttggtggggggggcgggggcaggggaaacagctggccctggctcctgacaggcactctgttcccatctcagctgcctggccctgcctgtggcatcctcagagacccagtggtgagtacttccttggccctttaggcccctttccccaatccagtagtgagacacggggaggggctggcagagacacaagtctgccaccctttcctggacagccttctcctctctggcagcaggaaaccaccccctactcccactccagacctacgctcacactccccgtcaaccccaagtgccactccagtctggggtccctccctgagctttcataagcattgcaaatgcccaattccttcattggctggggagggcaaagcctaaatgggcagtgggctatagatggtggccttccaggcactggcagcaggacacttcagggcatcattcaaatgcagaggggagagcattctgcccaagggcaggcctatttggggtgggcagggcagacttacaatggagactaccatacttacgtggctctctgcaggccacgtgaccccttccctctgctccatgtgcccaggattcaaactcaggctagtctgattcccaaaactgtgggcctcagttttgggaaaactgagaaaattgggcctcagaatagggatagggatggagatggagatggaagaaaataaaccaggccctctgcctgatcccctccctactttggctctggggttgggagatgctgcagaggcttcatccactctctgccccgaccccctaactccctctcctgctctgtccactgggctggaccctgtctgctgggcctatcagggctccccgtgtgtcctccaccagccagcaatggccagagagcccagctggccccagcctatcccctgcccaccctgggccctgctcagaggacaaggctgaactgattctgtcaggaagggttagtcttgtctcaggcctggctgaatgcaaaaggacgagacaaatgcatgtggacagcctcgatccggagtgctctgtcaaacatgctggccacctctccgggaagatcactcagactccttgcatcccatcttgggaaggctggcctgaggctccagagagatgttgggagatgccaggtgcctgcaagccaagggccctccggggaagggtgtcaggacccgctgcttcttgcctcttgcagagagggcaacagctggctccttctcctgccccaggtgaagactgatcagatactgattccaggatgaatccagcaacattaggccctgcctggtcccatggtagcatctggctgggcttttggcctatagtcgagtagatagagggagggctgataactaagtgactacaacaggacctccctgtgtaagggttctagcttatttccacactcttctggcacagacaggttggcagggcagctggtagcaggcacagcagatggaatgtcaggaatgaaatccacaagactccaagcccctcccagggtgagcttgccacaggccacaccaggaagctgagctcctcacccaaagcctgccagtgcctacgtggcagtcattcccttgtcaacagacttcccactggcttctcttgctccattattaaaagcaagaaccaaaaacttcactgaggggatagaatcccactaataagcatgtccacgctttgggtcatcttaggggcacagaaaatctgagtatcccccatagcatcaggcagagcagatttcttgtcaattagctcagttggttggaggctgcagccatgaacatgctggcctcagggccctcacaacacaggtgggttagggaatgaatgaaagttcagctattattcctgccagagactccaggcccaacctggcctctaatgagctatgtgactttgggcaggcagaaaagtgtctctgctatggtttgaaggcatcccccaaaggcctccaatgcaacagtgttgagaagggggacctttaagaggtacgtgggtcacgagggctctgccctcacgaatggatttgtcctcatcgcaggagcgggctcattatcatgagagtgggttggttataaaattgaatgtggccccattccttctctccctttcccccatgtaaggccccaccagacatggccctccagtggtggagttcccaacctccagaaccacgagacaaatgaactgttattttttcataaatgactcagcctgcagtactctgttacagcatcacaaaatatgctaagacacccctgagactcatcggccacctgtctgtcagctttgccgcaaaaatgatttgggagcagctatagtcaactttcctcagactacctcctgctggcacattacggccacctgctgtgtgcctggtgtttttctaggttcttggaataaagtgaacaggccgggtacggtggctcacgcctgtaatcccagcactttaagaggctgaggtgggcagatcacctgaggtcaggagttcgagaccagcctggccaacatggtgaaaccccgtctctgctaaaaatacaaaaatgtgctgggcatggtggcaggtgcctgtaatcccagctactcgggaggctaaggcaggagaatcgcttgaatccgggaggcagaggttgcagtgaaccgaggtctcgtcagtatactccagcctgggcaataagagcaaaactccaagtcaaaaagaaaaaagaaaaaagaaaaaaaaaacagaaagaaagaataaagtgaacaaagcaggcaaaaatacctgcccccagaaagatcacattcagtggatgtgcccatgttccttcctcttcctaaagcactcacaaatatcaacattcccctgaactcaccactgagagtcacttctcagcgtgacaatgttgaccagtcttcacctgctgtgtaaagcaggcctgccctatagtgaccccccaaacaggtgcagaaggcaggtccaccatcagcccatggccagcacaccagttagatacctgctgttgccaggcagctgtctgccagcccgtaggcactagagagccttggggcctttcctgagcccttttaaagaagtgcttcctttagacatggactctcaggcctgctcagagcggggagggtgctgctccgccccaaggccttccctccatcctttcctctggcaatcaccgtgatagcagcaacgcccagcctgaggccttgggtgctgcttccctcaggaaaactggccaaagagtagacagcagatactttctccatattcccacccccacaacacaggccagcagcacagcagctgtagacaagatttcataaagaactggaggacaagaaaggggagattaaggagccctaggctctagggcctggctcacacaagaatggtcttcctcctgccaacttcttggcctttacaaaggccccacctgacccagcccagcctcctgggcactggccaagttccacaccaagactaccagaccccgccaagtccaggctcaagagagaaagccagaccctctctccttccacctacctgagagaggcctgtcctcaggtgtctggctcacagatgggggacccaactgggagtcactcccttctggaagccccaggccctataaacaggtccccggcctcactgctgggcctcggcctggagacagcggctcagtgcaacacaaccacgccagagctgatcaggggaaacccacgcagcagttgagggggctggggctttgcgggtgcagtgaggggactgcttcccttcagtggagttgctgccgcaggtgaggggaggaagatgaatctgaaggttctaacgtcagcccacaggctgaagcgaaggctggcctgaagtgttccacctgggcccccacctgagcggctgaagagttctgtttcctcaaggcccacagagctcctcagagggaagctttagtagctatttccttaggaaggcaagtatgtgtgcaacatttcctaataaaatgcaaaggctgttggaggcactccagccccctcatttagctctgaggtgtaattaatacataaattaagctcctgcagactcacaaatgtccaagaaaaaaaatgaaaaatataattagtttgcccatctgactccatctgtcagctctgagctcaatgctcaacgtagcaaatcctgagggctttggtctcgcattggctgggcccggcgctgctcctgccctgctccagggaagtgggccctccgcagcagctgcccagagcccgggctctggactcagcctccagggcagcaaggactctggagtacacagactgcaggctgggagccggggcactcggcagagcagccaggaccttctctgtggccctgagcgctggctaggggtggcaagggcaccccggggaatttgcatgtgcctcttgctgccatgttcctatttgtgcagacctggaagtgggcggcagagccctgaatgggctaagactcgccctcctcctccaacctgtcacctctgtcgcttactatctgtaagaagagaaaggctgcaaaggggaaagggaactcgagaggaaggcaccgaggagaagagaggagacaagagggcattgggggagcaaaggaggccactcacacaggaggattagaattaacaactacccccaccccagctccccccaacacacacttacacacgcttgtattacaccggggatctcacacacatccggtcactcagtattcagcactctaggaaaagaacaaacaaatggcttgtctttcatcccccaaaattacttatgtcataaaattaatccatatttgtttcagaaacatttaaataagacataaggggtatggcattaaacgtacccccaaatgcctcttccccttacaagtggggatcatcattaaaaacttttgtcccagaagtggtgactcaggcctgtaatcccagcacttagggaggctaaggtgggaggatcacttgaggccaggaggtcaagaccagcctgggcaacactgtgaggtcctgtctctaacagaaaaaagaacaggaaaatttaaaaggaggaatgaaaacaattgtttaaataataatttggtgtgtattatttaaagtacattaatatttggtgcaatcgtttttgaaagggagattgtactatacatgttgtttggcaacttaggtgctcgctcaggcaccaggccacacacatcttgggcattccacatctgggttttgaaagtaaaattataataagaaaataagcaaaggtcaaaatgggttggaaaaaaattatttacacagtgagttttctactttatcataccaagaagatgacttctctaattattattatttttttttaagatggagtttcactctgtcacccaggctggagtgcagtggcgcaatctcagctcactgcaacctccacctcctgggttcaagcgattctcctgcctcagccttccaagtagctgggatgacaggctcccaccaccacatcaggctaatttttgtatttttagtagacatggggtttcatcaggttggccagggtggttgcgaactcctgacttcaggtgatccacctgcctcggcctcccagagtgctgggattacaggcgtgagccaccgtgcacccggcccctaattattatttacgtagaacaatatcaaagatatttttcttcctaactctatactccttgacaagaaaaaaaaagaaaagaaaggaagagcccgcgatatataatggtacatttgaaaatgaagcatttgtatgtgcgctcacatgagtacatatatatgtattacatccatatgcatgtatgtgcatagactgtttctagaagaattcacaagaaactggtaacatttgttgcctctgggaaacagattggcaactggtagaaggtgactgacttcattataaacatattttgtaatgttttaattttctgtcacatgcattattacttttttccttagaaaggattttggttttgtctattttatcggaagaggcaaattttaacaataaaaacaaaatgactgacattcagccatagtcccacctctctgaattactcagatctactgatttacatgtagattaaatgcaaatgagggtggcactatggttgattatttagcagctcagcaatatgaccaatgccctgttcccttctacccttctgctctgccccctcggcttgtagactttgtccttgggcttcctaacctcattgctacaagagtcaggcacatgatcaaaacaagacactgccccaaggtaggaaagaagccacctcttccttcctatttttaggagaaaaatctttcccccaaagccctcctgcagacttagcccacatatcccactggccatatttatttcccatgtctatgcctgagcccatctctggcaagggaaagggacccctttggacatcccagggcaagcaggcctcctcagtgttatcagaccaactacccctcttgaaaacaatgactgtattcttgtaaccacccaacctcaatccagaaacaaaattcacagatcacagaatgtacctacaaaaacatatttcaaaaaagaatcaaaacgaagccctagctgtcacgtaaaggagaaagtaaaagtaattcaagacaaaagagtatgttttataataagcaaaggctcaggcagcagactctacagaacataatgaagtggtgagatgcttgcagctgcacacggaaatgccaaggttgccccatcatacatatagacagatgcgggagatgactcacccagcacgaggccattgccactggcggcatgattttccaaaatggcgaacaactgttggtaaagcttcaaacaaaacacagtgcaacctccccttgatttatacagtaggatacttcctaggaaatttagggcatatttaaaaacatgcaaaaagatacttggagctcatacatcaaatggaatgaagttttaggcttaaaaaggactttcagctacataaaagtctcatgagacatcagaaagtcacgtgggacatggagccatttcctcttagaaaggactgtctcacacattgtaaggtatttagccttcctagaccccgtttattgaaagatgatagcaagggccctccccacaaaatgtcaaagcccccaccaatttccagaacacacttagggaatggttctgtccctttcgagaactcctggcttaggccaacctgcctctcccagcccaggtgacctcagagctgggtggttgtctgaacaacattcaggttcttgcaggaagcatagaaggcgattagaggttggatagatagtcaaccgtctgttcatcacagaagcatttgcacatatttgtctctagctttgcctttgtatacatatttttaaatacaaataaaatacgtatttaaaatatggccaggtgtgttgactcatatctgcaatcccagcactttgagaggctgaggtgggaggatcaacttgagcacatgagtttgacaccagctgggcaacatagtgagatcccatctctataaaaaattagccaggtgtggtggcatgtgcctgtggtcccagctactcaagaggctgatgtgggaggatcacttgagccctggaggttgaggctgcagggagcactccagcctgggcaacagtgaaactgtttcaaaaaaaaaaaaaaaaaaagacaaacaactgatttaccgaatgggcagaacttcatggcacatacttggctttctttttctttttcccccatcactgtttttcagtgctggctggtcagctctcctctttcagcccttggatgacagctggggctgtcccatagcttttcagtcactttgagctatgtcataactgtggtcatattttcaagcatgaaaagtacagatttgtctcaatcaaaactcagtctcacaaaagccactttcctccccagctcagtgcccacccagtgggcaggaggaggggcaggtctgttccttttctccctcgctgccaccctcagtccaagttcgggtggggaagagttgattagaggatggggcaaagtcttagagggccggacgtgggtgatccatcacacatacccattctatcttggcctctagggtggattttgaggagccccctccccatgatgatctctgcactccacaaggagttcctcgatgtgacggtgcactctctagctggtcttctgtggcatgccccctctggtacccagtggttcacccctcccaaccagcctttccctgggggacctctcctacccagcaggtgagtctcctgagtgggcttctttcaagatacctgaaaacccaactcctgggaggttcacttgctccaaggaaacttgtacatctttgccttgcctaagagtggaggagcagcttccccacaactgaccctgtaatagaaaaaaacaaggtgttttgcctattctcacacacagaactcccaacaccagatgtgtggagcatttcccacataccaggcaattctcccttggacaccagctgggtgtcctctaattcagttcagttttgatcctatctacctggagatagcatcagatcccacaggtcgatggctcagtcctgcgagactgcccctctagagatgccaatctcaagtgcaggttgtgacctgtgctttgaaccattgactatatattggggctccctcgactccctcgttgggtgagattaatctgctagagcagctcataaaactcagggaaatgctctatttatgtttactggtttattaataaaagatgtaataaagaatacagataagcaccagatgaggagatacatagggtaaggtctagagggccagcagcttctgtctctgtggagttggagtgtaccaccctcccagcaagcagatgtgttctccaacatggaagttctctgaacctcatacttagctgagggattttttttttttttttttgagacagggtctcactccgtcacccaggctggagtgcagtggtgccatctcggctcactgcaacctccacctcccgggtacaagtgattctcgtgcctcagcctcttgagtcgctgggattacaggcgcttgccgccacacccaactaatttttgtatttttggtagagacagagtttcaccgtgttggccaggctggtctcaaactcctgatgttaggtgatccccccacctcggcctcccaaagagctgggattacagatgtgtagctcagggatttttatggagtcttcatcacataggcatgattgattttcaagtccatttccagcccctttctcctctccagaaaatgggtcatggggctgaaagctccaagcttccaatcatggcttggtctttctgctgacaagcccccatccagaagcccaccaagagttgcctcaatagaacaaaagatcctccttttacccaggaagttccagggactttaggagctctgtgtcaggagctagggtcaaaaaccagatattagaacaaaagattctcctagcacctgtatctacaaggatattaggagtgctgtctcgggaataaggacagagaccaaacattggaacaaaagattatcctgacacttctatcacttagaaaatcacagctgacccttcaacaatgcaagggttaggagtgctgacaactgcccaactcctgcctcgcacacggttgaaaatttgtgtataacttttgactcccccaaagacttaactacaagcctactgttgactggaatccttatgggtaacataaacagtcaattaacagatatatcgtatatattttatatactgtattcttacaataaagtaagctagagaaaagaaaacattattaagaaaatcataagagaaaatatatttatggtgctctacggtatttattgataccataggtctgcaccatgtgtttacaacatgtatcatctgtctgaaacgtcaggcaacctcagctgctgaccttagtctatggtacatatcgagcaattcaacttgttcctgtgaggtcatgaccttacttcttaggagctcttccagcatcactagtggcacttcgtatgggtcccatgatgctattcaaggtttacagtattgcactaaccatgaaaataaatgagaactgagagagatcatggttttcactgcaatatgcaatttactggagattgcaatagtaaattgtaaattgctatatgcaatctactgctcacgtggagatggtcagtatttcatagtgttttaagcagacactcgcaacacgagctcaccgcaatagcaacaggaggtagctacaaaaatattacagtagtacagtgcttactacagtgaattttatgtagttatgattgaatacttcatctttacatttgtttacatttccctcaactacaaatggtgccatgtatgatctgtgttttttgcatgttttcataaattttaactttttataatagatttgtgtaattttatagtagtaacttataaaatagactagtactcgcatatactttatgcattcatgacatacctaactttttgttgttcttttttgatatttctgggctacacaagggcagtgacatgatcgtagcgcactgtaacctgtaactgtaagttttttcaaactgtcacaaatctcaaaaaaaaaatctaatatatttaatgaaaactatccaagtataagttgacccttgcagttcaaacctgtgttgttcaagggtcaaccttacaaggattttaggaactctgctgggaactggggacagagagcaatgtgtatattttgtgttatttcaggtgcgtttgctcatgcctataatctcagcactttgggaggctgaagggggagaattgcttgaggccagcagtttgagaccagcttgggcaacacagagagatcatgtctctaccaaaaagatcccaaaaattcatattctccctcctcactttacctcacacgatgctccaaatcagtctcccaccttaagaattctccataagcaaaagcccatcttgaatcccacactctcagggacccttgccacactatccccccaattttcttttctttctttatttatttatttatctttaagacagagtcttgctctgtcgcccaagctggagtgcagaggtgcgatctcggctcagtgcaacctccaccttctggattcaagcaattctcctgtctcagcctcctgagtaactgggactacaggtacatgccaccacagctggctaatttatatatatatatatttagtagagatggagtctcaccatattggtcagtctggtcttgaactcctgacctcagatgacccacccgcctcagcctcccaaaatgctgagattacaggtgtgagcccccgtgcccggcctcccaattttctcattggccagtcactctggtgccctggggactagcatccagctaagggatagatgccagctgctctctcagtgctatggtttgaatgtttgtgcgccctccacaattcatgttgaaaatccccagcacaacagtatcgaggggtggggtccttaggaggtgatgaggacatgaggcctctgcagtcatgaatggattagtgccctatagaagggctttagaggctgagtcccaccttccatccctccaccatgtgaggacacagcatttgtcccctccagaggatacagcacaggtgccatcttttaagcagtgagcagccctcaccagacaccaaacctgccagtgccttgaacttggacttccgtcctctagaactgtgagaaataactttccattatttataaactacccaggctcaagtgttttgttacagtggcacagacgtactaagacttgtgggtacccacaattccataagccattttctttgcttggcctgggcatgaccccctgtgctgtccccgtgaagaaaaaggataaaccaacaaactctatgaattaacttcagtgaatctcgtatgtttctaatcttctgctttggagctttggcgataaagaaagattaggaaattagattaaaaaaaactaacattatcttaaatgacccatattcccaatcaatatctcttcccttgaaacttggtgtgtataaatccaatactttttaatgtatttgtatatacatatgtaaacatatacaaatatatacatcatatatatgtatgtatctgcctaaataaccacctggagttagcaggttccatacctgcaggatgcaagcagggatgggcagaggcaggctggtcagcttccaggtatatatatatatatatactcggaaaatacttgctattagtttggtggttttattattaacgtaaatgatgtcttgctgcacatattattctgcaagttggttttcccacataatgtgttggacatcttttagccccatttcacactcttagcctgccttttgcacaagagttgctgtggcaacaagctgcccccaggtgtaacctgacagcttcacttcagctgcaccaggtatgtctttctttctgccccaggcatcacggtatacaggcaaatcttgaagtgccagggagtcaaccctccctggagcaatccttgcccaataggagaaggaagccaatgaataaatatgccaggcctcgaggtcttaggccctcagtctaaggcatagtctacatggtttctctgggagtcctcagttaaactgagccccgactgcccacagcaatggccagtgttaaaagaacaaccttaaccagattaaatttaacacagtttaactgagcaaaggacaattcgtgactagggcagtctgctgagccagagtaggtttagacagactctggtggaagatttatggacagaaaaaggaaagtgaagcacagaaaacagaagtgaggttcagaaacagctgaatgggtcgcagctcgtcgtttgccttatttgaacacagtttgaaaagttggccacctttgattggccaaaactcagtgattgacacaagagtacgttacagcctgtttacatttccctgtaggctatagtttactatgcacagagaaacctttaggctgaacttaaaatatgtaaggaggaagctttaagctaaatttgatttttgttgttgttgttgttttgttttgttttgttttttgttttttgagatggagtttcactcttgttgcccaggctggaatgcagtggcatgatctcggctcactgcaacctccgcctcccaggttcaagtgattctcctgcctcagcctcccgagtagctgggattacaagtgtgcaccatcatgcccaattaatttttgtatatttagtagagggggtggtttcaccatgttggccaggctggtctctaactcctgacctcaggtgatccatctgcctcagcctcccaaagtgctgggattacaggcgtgagccaccgcgccaggccactaaacctcagcctcccaaagtgctgggattacaggcgtgagccaccgcaccaggccactaaacctcagcctcccaaagtgctgggattacaggcgtgagccactgcgccaggccactaaatttaatttaacagtagcttaacacatggctggatgggcttttcctacttccttgtttcattcttcccagtcctccaactcttgactcctagatttatggacccaaataaatgactcaaatgcagtccctttgtctcaggtttttggtggaatccaagctaaaacactaaataaaaaatgttgttaaaaataagtttattttggtcattattgatccacttggagtggtcctcaaaatgggatctagggaatcggtccctgaggctggtttctgggaatccacaaatgcaaaactatttccatagtcacactaaggaggtgtggccttttcaagaaattacattacccgtaaagtgcaatagacggaaaacagaagcccatatgagaatccagctatcttctagcaagccagaagtaaaagatgccaggtgcagtggatcacatctataatcccagcactgaggattgcttgagcctgggaatttgtgaccagcctgggcaccatagtgagacccccatctctacagataatttttaaaattagccagatgtggtggtgtgtgcccatgatcccaactactcgggaggctaggaaggaggatcacctgagtcaggaggtcaaggctgcagtgagctgtgattgcactaccacactccagcctgaatgatagagtgagactctgtcaagaaagaaagaaagaaagaaagaaagaaagaaagaaagaaagaaagaaagaaagaaagaaagaaagaaagaaagaaggaaggaaggaaggaaggaaggaaggaaggaaggaaggaaggaaggaaggaaggaaagaaagaaaaagagatctgcaaaaaaatgtaaaacaataccactcttctcactaaatttttgtacttggaaaacatagtcatttttcacaaaaatgctatttatattaacataagtattttgatgtgcaatggacttatttttgttattttcacataaagattgtttaaatttcttagttttaatttctaatactgtaaatatcagtagatataacccatgtaaacaaaagctttttggggtcctcaataattttcaagagagaaaaagattgccaagactaaaaagtttgatagctattaatagagttcattgcttttaacttctgagtcccatgcatacttcattattattattattattatttgtaaagacagggtcttgctgtgttgcccaggctggtctcaaactcccaggctcaagtgattctcccatcttggattcccaaagtgctgggattacaggtatgagccacagtgccctgcctcttcttttaaaatctaattatgtatttgttatctcttacatttttcctatctttcttttttctgattatggcccaaaaggggaagatttctgcccatgctcatgttactatcttgaaattatttcttttcagactctctagttcaggaaaaaggtcccctgccacatcaagtggccacggaggagcttgggcctgtgatgtcccctcccttctgcctaaatgaccacctggaggcagcaggttacatacgtggaggatgcaggcggggatgggcagaggcagactggtcagtttccagggcacacttcgggggcatacaagggacaaaaatgtgacggtggctcaggagggcaccaaagatggccaaaagcttctgcttttaaggtggcagtaggaggcagttaatggaaaaatagaaaccaatcaagctacgtctgccaccaatgaaaatgaattattatttattcaacaaaatataagctttttaagaaaaaaaaaatagcaaacgctcaacccacttgctaagttccgttggggtataatggcatcagctctgagtgccaacttaccttggcaaggtggttgcacgccctgaggatatgtacaccttctccgctgctggccagctagatgaccttgagcaagccacccacccttctgaacttcgtctatttggaaaatacagacattaatacctcatagtttgtaatgaggccttagagtccacatattaatcgttcagaacacagctcaaaaaaatctaaaacttctggtggggcacagtggctcacacctatgatcccagcactttgggagcctgaggtgggaggatagcttgagcctaggagtttgagaccagcctggacaagatagaaaggccccatctctatacaaaatataaaaattagccaggggtggtggcacatgcctgtagtcccagctactcaagagactaaggtaggaggatcgattgagcccaggagtttgaggctgcagtgagctatgatcacaccactgcactccagcctgagtgacagagcaagaccctgtcaccaagaaaaaaaagagagagagagagagagagaggtggcttcagtgggccgaatagcagagatccactgggagcattagcaacatcagggccattgcttcagcagcctggaactaaagaggaagaaagcaagaagcttcaggagattgaacagtaggaaggaaggaaggaaggaaggaaggaaggaaggaaggaaggaaggaaggaaggaaggaaggaaagaaggaaggaagggagggagggagggagggagggagggagggagggggagagggaagcatatgagaaagcgaggctaggagtcttggagtcatccttgactcatctcttattgtagtcattgccagtactcacagctctcagacaacagtgaggaaacaagttattttcaacatttcaggaagctaaatggaaaccatgcacacaacaaggtttcaaagttaattgaggatcaagattttctgctgttgtctggtaagttatcaattctgatttttttaaacctgattacaggcatccacaaaaaaatatgcaactaacatcatgtttaatgttgaaagactaaatgctatcctctgggattgggagcaaggaaagggtatccactctcatcactcctgtgtacttaaaaaaaaaaaagaaataaaacatatgttcacacaaaagtctgtatgcaaatgtttatagtggctttatttataattcaccccaactggaaacaacctaacatgaccttcagctggtgaatgaataagcaaaccatggtacatccatacaatgaattactagacagcaaaaaaagaaactggtgatataggcaacaacctgatgaatctcaaatgcactgtgcttagtgaaagaagccaggcccaaaggactacatactgtatgattcaatgttgtgacatactggaaaaggtaaaattataggaacaaatcagtagttgtcagggtctagtggctagggaaataattgactacaaaaggacagcaagggggaattttggggggaattggaactgttctgtatctcaaagatgggcatgattgattacatgattatgccaaaattcattcatcaaaaatgtaaatttttatgtatgtagatagattttattttaagtgaataaaaggaacaggcaacttggaagcaaaagagcaggagccgggccaggcctgcggctagattcagaaatagggttagagggaatgatcgaggggaatgacccaggcagccacagctctggagagacggctgcaggtggacaaaaagcaggtggaagcgcccagcaggaccttgaccagacaggcaatgggaacataaaagacagcaacagattgcaacatcatgtccatgggctcccaaacctgatatctcagaatcacttccagagctgattggaaagacagattccattgctaagcatagtggcctgcacctgtagttccagctactccagagggcttgaacccaggaggttgaggctgcagtgagctatgatcgcaccacagcgctccagactgggtgacagaatgagacaagtcagaatgaaagaaaggaagaaacaaagaaagagagagggggaaagaaagaaagaaagagagagagaaagaaagagagagagagaaagaaatagaggggggaaagaaagaaagagagaaagaaagagagagaaagaaagaaaagaaagaaagaaagaaagagagagagagagaaagaaagaaagagagaaagaaagaaagagagagagaaagaaagaaagagagagagaaagaaaaggaaggaaggaaggagaaagagcaaaagagacagagggaaggaaggaaggaaggaagggagggagggagggagggaagagggagagaaggtgagaaggagggagagagggagggaagagggagagaaggtgagaaggaaggagggagggaggaaagaaggaaggaggggagggaggaaggaaggaaggaggggagggaggaaggaaggaaggaaagaaggaaagaaggaaggaaggagggagggagggagggaagaagggaaagaaagaaaggaaggcagatttctactgaggctgcctctttattcccctctttattcccctcagagaggtcatttaacctctctgatctctgtggttgctgtagggactaaaggacacaatccttgggaaagtgcttggcacagaacttttcagcttgctatggggagtgccagccaccggcaacaggtgtgactgcaggggaaagccccgtgcaggtgagagaaaggggccagaatgagaagggctttgtaaggctgggaccactatgttgggtctgctatggaggggggagctcaggagtaggcagggcagatttgaacatgggaaggggaaagctgctgctggaagcagtggcccatgcccaggtgcatggagaagagagcctggaagcctctcttggtccctctaggctggcagaggcagggccccacattggcagtggtgtgaacaatttcccctcatcatggggctgctcatggctgtgaaacatgggatggcactagatttggtcattaagtaaataggataaagaaataattggaaaatggatccctgcctggaaggagtggaattgttcctggggctattgttgtccggcatcctgctttctgagtaatctcccgagccaggcaaggaaggatgtcaggcttagggctgatgagaaacttacacttgaaaagagctcaaaattacccatgacaatggaaaagcctttgtatcctgaacagagagaggaaaaagactctgaatcttctctggggcgggaggagagggaagataggagggcagtctcgagtcacagaatgtcaaacccaggacccctgtgtctttccaagttgggggcactggccagggaaggatcctggttcttccggtgtctgggatgatgaggctggtcctcaggcagtgggaggctggaggagggggaatgtgtgaagaccacatgctttcgcctgcaagagcatccatccatgtcttggtcactgcaatttacatcctcccattcaaccctcagcacacccaccaaaccaagctcctccttgcaccttgatgggcagaaagttggagtgatgattatctaagacagagccctgaggtgaagaactggttttctggtggagggggtgaggggaggatggagggagctatcccttccctgtccccgcaaatccttcagcaaaaaatccatagtgttgagtcaaaatgccaatcagatgtgcctctcagcagctttaggcctggactttgacctcagccccaacccccacccccacaggcactcagcctaagctcccattctgcaccctccccaccctggtttgcaaaatgcatcaaagcagaagctgtgcttttgcctttccttgtaacttcttgtctgcaagctgtattagatgcttgctggtgtttaaaacattttctatttctctaaaaaaacaagttggggcaccccaaaatgaagcctcccaagttcggtcaaacatgcaagattgccactcaatttctccattctagatgagaatagcgagtcccaaaggctcgtgggtgtctgcggcgttgcactcacttaactcacttgttcaaaatacaggtgccagggcctcccccccgagccacagaacctaaatccccagaatcaataactttctttaaataactagatgcttgatttaaagggaaaactctatttaaagtgctgtatccctccttaaagacagattgtcctacacgaagcagctacatgcaaaatgaattataggagtgtccttggttttacaaaaaatgaaaagcattgcaattctccaaacatgataagaaaggaatttttttgttaaactctgagagcaaaacaatttactagaagatacagatgcaagttgagtaagcaagccaataatggagaaaggaggaatttttacagaagcagtttttcctcaccccatctgcatttcatgtctattaaaacacatgtcatgcaacgaaaaaatcaaagcagtatggttgggtacacacattagtggctttctgtatggttagtgagatgaaaggacagagaaagctatgctggagtcatcaggacatggtagagccctcttgtgagctgctggaacacatcagttgcacgtacgttctcttgctggtgcagcccatccggtcagaatctgtcgctcacagtagctgttactgaatactgtctgcttcttagctgaaaccttaccgtaggaccatccggccctgctcctttcccgttaacatgtaagttgttctcagaccctaccttttcccttggtttctcatggaccgtggccaggacttgggtgtccacagccacacccacaaaagaagtgcctgttccacccaggacctctgggttaaatgacctctctgaggccacacaggatctacagcacaaactcagctcagattataaaacttgcactctgaacgactgccctagccagccccaccaaggccatgaggggcctcccagagccaagtgcgacggcttctcttggcctctgcctccttgacctgcaagcaatatttgaccccacttcccttcccgtctcacaccctcctgacctcctcagctctcctttctggtctctcttcccctctcctcacacctccaattggatcttcagccttcctctcctctcactccacatttttcagctggattcctgtggttttcactcttatctctccctatgcctgccaaatctgtgtgttcagctgtccgaacttctcagctctacatgtgtgtttgcaaaggcttcaccatcctccccacacttcctctaccagaggctgcaccatgggtcactgggcctcaatcttcctgcccactcaggtagaggctctagggtcagctttgcctcctgccggatgctgcactgctggaatcagctgcccacacttctctccttttgtatggtgtcctcactgaaacttgggcggtgtcccttggagtgccttgagagctgatgattgccaccctctctaagagcctacagacccctgatggatatgtgtcctggagcccctcacacaagcccagccctctgtgttagctagacagtcaagtgggtgcacacagggctagcaccaccgcggctcagaagccaccttcccctggaagccactccttatatctgggcactttctcctctggactacccttgcattgtgttgttctataggaggtgctggagacagtgataagaacccatggcccctgccctcaagaagttctccagttttattggtgggagcaaacaaataaacagacagacatggagaaaatgcatcaggtcgggtttgcatgcaggaagttgtcacttgcgagggaatgacagaagcaaaagtgggcagagggagaagttaaaccaaggtgcagtcccaccagaggcctcagaagaacccatgagaagctctgaaactggatgggctttcagagttgtaccaaatggaggcaagagggctggcctctacccctgtctcatccactagaatgtgggctgctcccagagagggtgtgagtgagttggggcaatacatctgccttcagtggaaagcaattaattcccagagagggttgcagccattgctcctccagcagccagggatatgagtacctgtgtgcagatggggacctgggcaacatgccacagcatccatgacacggcacagacagggatgcccaaagtgggggctggccaagtcaaagatgggggaagtaatgaggtgatcatcagatcttcccatttcacctgggcattgacctacatgtaggtgtttttcaagtggacaatctttagttccctctgctcctcatcatacaagtataccagcaatggctaatgttatctttgcacttgctacatgcccagctgtgtcttagctcttcacatgcatagcttatttaatattcacaagaaccctacgaattaagtaatactggctaggcacagtgggtcctgcctgtaatcccagcactttagaaggtcaagtttggagaattgctagagcccaggagttcaagaccagcctgggcaacatggtgaaacctcatctctacaaaaaatacaaacaatttgctgagcatgatggcagacacctatagtacctgctactcaggaccctgaggtgggaagatcacttaagcctgggaggcagaggctgcagtgagccaggatcgcaccactgcactccagcctggataacagaatgagaccctgtctcaaaataataaataaataagtgggccaggcacggtggctcttgcctataatcccagcactttgggaggccaaggcgggtggatcacttgaagtcgggagttcaagaccagcctggccaacatagtgaaaccccatctcaactaaaaagacaaaaattagccgggcatggtggcacactcctggaatcccaactacttgggaggctgagacaggagaatcacttgaacctgggaggcggaggttgcagtgagctgagatcatgccactgcactccagcctgggcaacagagcaagactccatctaaataaataagtaaataaataaataaatacataataaaactattgcccctgtcttaatgtaaatggaacatgaggcttagagaggtaaaatgactcaaggtcacacagtaagtcaaaggctgagctatctctgccctttcttcttcctggtgtacacctttcaccctgcaaagctcagtgcctaccttacttgctctatcaaaccttccccatttccctcagaaagaccaagtccccctcctggtctctagagaacaagctgccctggaccacacaccttggcttccagccccttcaccatcctagacgaggtcacctagaaggttccatccagactgtgaatgtccctctttaactacagtctccagtgaacccacaacctcagggacatatgaccttggagggttaagtgggatttcatttccctgactctggacctgggctttgctagcacacctctcccactatgatagcattcgcttaaggcagctgagccccgtcaggggctccctgagcttgtggtcagctcaagccatcacatctttctcatctgtgctctgggaagctgcctctgcttggtctacacagctgagacaatggagcctgggtgggctcaggggccccttctgctgggaacctgctttgaaacaacatatccattgcattcactggaggagcatcagaagtagacggtaatttgtgttttttcttcactggatgccctagggtgggttcccccgaagcagaccctgagatgagcatttatgcaactgatcgaggatgtgcccaggagaaccggataagggactcagagttggagggaagatactaagaaaatcatgctccaggcaaagtcccagcctcggcttgatataaattacactgacttgaggcaagggagctgggctctcacaccccggctccagtcagccactgtttcagggctgccccaggaggacaccagctcccagcactcttgccgtctgtgcatgcattcagcacactggcaccccctcaaaggccagccatttaagaggggcacaggtgctggggccacaaagcacccagagcccagtgatgggtgcacaggcactgtaaaagtgatatgagggccgggtgcagtggctcatgcctgtaatcctagcactttgggaggctgaggtgggcggatcaccaggtcaggagattgagaccatcctggttaacacggtgaaaccctgtctctactaaaaatacaataaattagctgggtgtggtggcacacgcctgtagtcccagctactcgggaggctgaggcaggagaatcacttgaacccaggaggcagaggttgcaatgagctgagattgcaccaccgcactccagcctgggcgacagagtgagactcacctcaaaacaaaacaaaacaaaacaaaacaaaaaaagggatatgagaggatcagagcagcgtccccttgcctcctgttaccaagaaggtaccaagccgttgttccctcaggttgttcctcattccatgttgctcagccagccaaaatcagaagctgccaagccacctggggaaacaaggcacccaccgaagctcttgttttcaaaagtttgagctaattaaagttgatggagctggatttcccctgaaggaaaggataccaatgtttgggaaggttaggattcagagaaagaagcatttgaagtcttccccagggcctagaaattaaagtctagagatttgggagggtctctagaagggcaggaacgcaggccttcacctgaaccacttcctaagcagatagccaacctcatatgggagattcctaaaggggctgacatcaaatcctgagactccatagtgaagaccccaagctgcagcctcgcctggaagtataggaacttaccagagtatccaatgacaggagaagtccagagggctctaccctgaatgttcaccatggataagtcctgccccaaacctgacagatacagatttctcatctatcttgacaaatgggaacttgcagccagtttaatttgcctcaaagaaaataaaataatgtgacattccttcacccccagcaagaactaatccctttctccagtgctcctacagcagggaggacatttccctgatattgatttttcatgactgtccttcctccctaggagaccctgatctccttagggaagaggtggtgctttttaaaccatttctgcagcccaggtgcctatcaaggccccagcatccagtgggtctcaataaatgagacctatcataattaacattcccctacctcagcacatgactcatcaaagtattacatgttgtttgcacttctctaccccctcccccttcccatcacctcaaagctctccaagcacacatgagccactgctcataaatgttgcatgaaagaagggatggatggatggatggatggatggatggatggatggatggatggatgaatggatggatggatagacagacagatggcctgatggatgaggctggtgagcaagtgaaagagtgcttgttttagaataagctatttaggccactgccatttcctgttggctgagcaatgagcatgccacagggccaatcagctctaaatgcaaggcggtgcatggccagaggaaagttcctgctgtctctccttcaccctgacagccatggccccaagggctaacagaggcccttgagaccaattctgccatcaggaaggaaaagccctgccaaaatatttgcattcagaaagttagcatttataacacagtcagagaactatgaaatatcttccaggccaagcactgggcaatgccatgttggtgggttctgatgagccctgggaaacttacttgcaccttttcagcccctaaccctattacacaggcccctgcatggaacaaatgccttcatcctcacatgttgctgttgggagtgtactttgatacagcctctactcaagaacaatttgggaatacctatagacattttactaacaaatgtgcacgccatttgacccaggaattctactcctaggaattaatcctgcacatatactcacgtatatgtacaaaatccccatatacaatgtttgtaaaaagcacggtgtgaaagccatctaactcttcatcagagggactgggtaaataaaggatgttctgcagccatcaaaaagaatgaaagttctacatgttctgatatggaacaatccccaaaatatattgctaagaaggaaaaaacaaggtgcagatcagtgtgcatcatttactaccatttgtgatggtcttatatagcacagaccagttctggaaggatccataagaatgggtcccaagggctaggtgcggtggctcatgcctgtaatcccagcactttgggaggccgtggcgggcagatcacctggggctgggagattgagaccagcctgatcaacatggagatatctcatctctactaaaaacacaaaattagccaggcctggtggcacatgcttatagtcccagctactcaggaggctgaggcaggagaattgcttgaacctgggaggcggaggttgcagtgagccaggatcgagccattgcaccccagcctgggcaacaagagtgaaactcggtcacaaaaaaaaaaaaaagaagaatgggtcccaggagttgcctccaaggaagggaccatggggactggggactgaggactgggagacaagagtagagaaatgcttctcatattgcacaattttgaatttttttgtatcatgtgtatgtattacttatgcttaatcaaatactcaattttaatattaatttaaaaaaataataatatccccatccacccagccaggtgccatagtatctgacattctgccaatagggagtcacaaggttgaagtcttagattgaacattgtttcaacatcatttctcactggaagctctttataatgcaagccttactagaactggtttaagtgtgatccaacctatcagaccaactttcctctttcgggcatacaaaaaacttcactgggcattgactttgtcctacacacttgcccaggcactggagagacagggagaacagggctgacctcacagtcagccaggtggacagctgcacgcacagagtggtggcagtgcacagggagtggtggctctgcacatggactgtgggccctgcatgggactagtggcactgtgtacagaatgatggccctgtacaaggagtggcagccctgcatagggacctatggccctgcacagggagtcgtggccttgcatgaggccggtggcggcctgcacgggactgatggcactgcacagagagtgtggccctgcacaaggagtggtggccctgcatagggacttatggccctgcacagggagttgtggccctgcaaaaggagtggtggccctgcacagggacggggcagatataccctgacatttgaggagactgaggcaaggatataagtgggagagagttagaatttcgaattctcagactccttgggtttcatgctggaacacgctggcatgagagaaccaggccttggcctgtggcccagctccctcctcttcccacctcctgccctgtcccaccccatcaggggcttgcacacacacacacacagacccttcagccctcagagccaagctccttgcacacagactctttccacagtcagcgcccttagctcctgtcaagcctccaggtacacacaaacctgcgtctctgctcgcctgcacaaggaaggacccagccaagtgtcccaatagggcctggaagcgatcttgatgccatccgggcagaaaatcccaggtggagtacagccggataaagacagcccaggtttcaggttggcatgttcctccggttctgcagatccctcgggccctggagaaggagatggccagaggaggatagcaaggccttccaaagtgcagtcccgggtcacagcccacctgcctggatctaaggacggtgtcgctgaggctgtggcagtgggaggttccaagacacggtgaaggcctgaaggagggggtggcccttgctaagacctgtggccagctgacagggctccccagcactctgtcagtaaggcccttccaggaagagggagtggctggacaagaggctgtgtgtggaaggcaaaggggagaagggagagagagggctggtaacccaattcctcttcctgcctccctacacttcagcatcaccctgttcacgccccttgcagatctcccacagcactccctgtgtacacctttgctgctcaaagggtctacacaagccaagcacagtggctcttgcaaaggtgggaagatgtcttgaggccaggagtttgagaccaccctaggcaacatagtaagactctgtctctattaaaaataataataattttttttttttttggagacagagtcttgctgtgttacccaggctggagtgcagtggcgtgatctcggctcactgcaacctccccttccccagttcaagctattcccgtgcctcagcctcccaagtagctgggattaccggcacctcccatcacacccagctaatttttgtatttttagtggagacagggtttcaccatgttgcccaggctggtctccaactcctgaccccaagcaattcgcccacctcggcctcccaaagtgctgggattagaggcgtgagccaccacaaccagccaataataaaaattttttaaagggtgatctaagatcagcagcatggcctcggaaattggaaatgcagaccctctggccccacctcagaaccactgaattagaatttgtatttggataaaagtctcaggtgagttctgcaaactttgtaatttgagaagcactggtctacaccacactccagcaccttaacgcagctccatttagcagtggtggacattgcatcatgcagtgcatggttccttcttgagtgcagcttatggaaggagggggagtgacaggactgaacttattgttcaaatccccttgtggtcttggggttcttggttgctcactgcagagtttataggtgaaatgagcagctgtcctcacctctccagcacccttggaatggaagaggctgccctgagctgggctgccatctcctgggtcctcagtctcaagccgctttctgatgtcagtgagctctttgaagtcaggtccgcatcctacacagggtaggtacagggggccgggaaggcatagaatacacattaggttggatctagcatctgatccacctccccatgtttcacgaatgggccagtgggtgagtcttagggagggctaggccctgcctcttagaggaaggcttaaaaggccccaacctactcttcttggcctggtcagctggaggcaggcagatgccccaagttcagacagtcagatactcccaccagggatttgggtctatggtgagtgacatctagaagcagggccagccagaagtccttcttggggctgggcacagtgactcgtgcctgtaatcccagcactttgaggggctaaggcaggaggattgcttgaagctaggagtttgagaccagcctggacaacatagcaagaccctcatctccacaaaagtaaaaataataataataataaccagccatattagagcattcctatagttccagcaactcaagaggctgaggtgggaggatcactggagcccacgaggtcaaggctgcagtgagctacgattgccctcctacagcccagcaattgcacacctaaccccacatacacataatcaaatagagattatacattcacgattgaagccaaagtgttatgacacaaaacttacccttaggacatgtcataagatctgatgttttatatttttattcttttttgttaaagaaagagcttgttgatccaaccctctaataaagctgaaagccaacatttgcaaacactggcaggtggctgcgtgtcagggtgaggtgtcactgaacagggtgataaagcaagatcttgtctctaaaataaataatataatataataaatataatataattaagtagaataatgaaatatcctgctggccaaggtggcatccccgcccccagcagcaaagtcagcagggccagtggtggcaagctggtgtccacaccagtggccacagtgtcatgtccaggcaatctctgccctatgacctcagctgcctttccagctgtctttggtttctgcctgtttcccttcagcctccctagattctgggaggggccggtgtcctgctccaggtaggcagagtccaagtctgttgtgggcagaactctgatgatcattgcaggggtgggaggagggatggagggagcatgatgctggcagaacctccatggcatctggggaattgaaggctgttttttgctcagttctcaccatccaaagcagcatgaacagttttcaccacgcctctcttgtggatgcctgtgttcctggtacacacctctgctatgacacttatcacctggtattagttaagcatctgcctccttcatttgctgtgggtatttagaaagcaatccatatttcctctctgtatgcctcaatttctccacctttattagaggacccaggataagattatttctgaggctgcttccatcagtggttctgtgagctggcccatgtattcagccagtcagtaggccatcaagcatgcatctacgcaaggcactagaagggttacatacaatgggaaactgaggcccagaaggaggaaagatgaagtgcccaaagttaaggagataaaagcagaagtgcaggtaagagaaggagtattcatattactgcaggaatccaaattctacaagaacatacgcttgttctttaaagggagccctcgctttaagcattatttgccgatcaatacccattaataattacaggacatgggcaaatattctagcaaaggtcctgaggtcttcctagatgtgcccaaaataacaaggactccccccgcaggtggcaccaaaagtcggaggatgtgttggaagctgaccttccccacctgtccctgcctcacccacagtcacccacccctctaggatgtaatggtgaatcattcaacacatgggccaccttgggaagtcgcttaacctctctcccttttcacgttataaaatgggcatagtcattacacaaatgcataaatcaacctcaattttctcatctctaagtgagaaggttggactggaattaacaagaggatccttgaagttctttcgagctctgacatcctcatccattgcaaagcggctgtgaaactggattatgtaaggtcctgtggccccaccaattgtttggcacatcctgtctttttccctagctagataatgatgtggctgagggcagggactgtgcccacactcaccaggtgcatgtgagtcagcgtctggattgtgtggccccgctggggctgccctggctgtatcacatgcctcttgtgcccatctcatacccctcagccaccctctgaccacagcattgcccggcgtgccccctgcaggcttttattctcctgcagccccagggcctcctcaacactatgatgtgagtcacctgagaagcccagtcccacaggcatgcacagctcaggagtgcaggactgtcaaggactcttgcagcaaccctcagccatgtggtaggtatgtggggtgcagggtggggaggcagtggcatacacactccccagaagcttcagtgggatggagatcagctcaacaacacacctttgacttgacttcccctccttctccacttcactcccccagcccctcaccctgctccctgagctctcctcccaaataaactgcatgcatgcaggcacacatgaccaggctgaggctctgctcttagtgggataccaggcaaagacacctgccctagggattggagcctaagccagaggaggcaggagggtttagggcaccagccaagaagtgctttccagatgctgagtgggatcccgaataagggaaagggcatctaacatagaatgaggaggagataacacaagggtagaaataaccaagaaaggggatttctctcagatggctttagaggtgctcccagccattgctgggctcacatggctcacatgtcataatttttgccaaggcggtttcactgccaccaccaccccgctcagcctccagtggcttgtccacccatggctgtggcctctaactgggcaggattttataggctaggaagaaaagtagggaatgttaagggagacggaagtcaagttcaacttggaaccatcccagaatgtccagcaggaaaggcccatggatgttataggctacaaccctctcagtgctcagcggaggaactaggagcagagagggtgagtaagtcaaaggcacccagaccttgtagggaatcctgtgtccctgcaggatgcagaatggatgcttctgaaccctgccacaccctcagggtacatgtcagagcacgtgtgtggctgggacaatcctccatctatgttcctcacttacaccggctccagtttaggaaacccaaaaggctacaaccaacccaccaccacagtgaccacaatgtacttggtgacacctcaccttgacacgcagccacctgccagtgttcacaaatgttggttttcagctttatgcgagggtaggatccaacaagctcattctttaatgaaaaagaatgaaattgtaaaatatcagatcttattacatgtcctaagggtaacttttatctcataacacttttgcttcaatcatgagtgtataatttctatttgattttgtgtctatggggttaggtgtgcaatgtagaaaagcttgaaaaccattgccttatacccatttgaaaaatgaaccaccaaacaaaggcttaaaaacatgaatatttgtcaaccaaaaggagaactcctgggcctggaagaaacatcacgattcaagtgctgcagcacgaaagaagttgaggctggatttgtattcagcttgactgtttgtaaagcaccatgcaccatgcaactgccttcagttccttttttttttctttctttttttttttttttttttttgagatggagtttcgttcttgtcgcccaggctggagtgcaatggcacaatctcggctcactgcaatctccgcctcccaggttcaagagattctgctgccttagcttcctgagtagctgggattgcaggtgcctgccaccaagcctggctaatttttgtatttttagtagagatggggttttgccatgttggccaggctggtctcaaactcctgacctcaggtacttggcctccaaagtgctgggattacaggcatgagccaccatgcccagcctgccttcagttctaaccactaaaggattcgtgttctcaactgagaaacagccatatggaggggcttcctgcaagggcactgaagatgtcagagatgatgcctgtggagcacctggcacagaggagctgccctgctgactgcagcacagccagttcacaggggtgagccccggcatcagcttagggagcatcccttcttgcagctcctccccctagcacactctcccacctctgctcgcacaggacctctgtccaggagtgcttacggcaaggtttctgcctcaaaaacagggcttttggggctaactgggagtgtaagggtaaaaggaaagcttccactaggcccccccgattgttcactggaaatgaagggacaataggagattaataggagaagaaggcatacaaatgtattaacatacataaacacaggagccatgaaaatatgaggctcaaagaagagccagatgactgaagtttaaatagcaccctcttcataggagagagggaaatgggggtaggtaattttcaaggttagtaaataattttgagggaaatcaaatggacccagaaggcagacattttcctgtaaatgtcctcttaggaagctgaatgggactgcaatttaagggaaagtgaggggcaaaactgcattgtgaacaaaggttgtcttattacataggtaaagtctccctagtcatccctcaaagctgccctcagaagaatagatgaaaagcctgggcttagtgacagcttttactcttttctccttgcaggtgggtggttaatctttcctggttatttgatgagatttctagggagggggccttaagacagttgcatttcttttggaaagaagtgtccatagtcagatatggaaattctagacagcctctttttcctgcacttggggagggggcagaggagaaacaagagaagattggaaaattcttggttctgaggcagcttctaaggacttgcattttcctttaattcaaagtgctcagcctgccaacacttcctactttggggtatcattctctgtgaccccacaggggaatggggtctgtcctcagagaatgggggaggacctcaaggcagcagggcagaactccagggtgagagtcaaagatggtttcctggacctgagactccagtggggttcatgctagagtcaccccagtcttcaggctcactgcccaaacacgcacttgaggaagtctgtgggggcagctccttgttttgttcctcactgtcattttccaaaaggcagacaagccctggccgccttatgcccagggctgagtccacaccatgctacacgagcacctgaggggaggctcctctccaaagtgcattaccagggcctgtgctaaatttttcagcagcctcttgtctcactaatggtcccgggaaatagactaagcctccatccccaactaagtgctcaggcctcccccacctctgtcctgcctgggcaagaagcccagcgccgccccacagcccagctctccatgcagcctcagctcccagtggccctctgccaaggacttgcgcctctgcagtgggatgcagtgcctcattttatccataattgtacgtggtccctgtgtgatgtccggggacgtgaaaagttgtgtctcatttcatttgggatccagttaaattcccaaatgctgcaactgagcaggaatctcagtgctcagaggccagcagtaggaggagtctggctgctgggcccggacacctggcctctccggcctctgaccagctctggccattggtcaccaccctggctttgttgggtattaatccaccacactctgcctggctgatccctgagtcagagccaggctgggatttgtgggtctctgagcccaccagcctgtgactcccaaaaagagagcacccttgctacttggaaggcttgccaccatgcctagccagcactcttgctttctaagctgaaatcattataagatagtcccctactaccttctacccagaaatgtatcccctcacaaagtatatgttcccaccctatcccaatcctaccatcgtgatttctgagtactttccagaaaactgtctccactggctcccaagggcagagcacttcctccagcctctggaatgtccaccatgttgatggtgcatggatgataatgaccccacggatgagctctctccagtgggatcacgaagggagaatgggatccctgactccccacagaacagctcggcccagctgtgaggttgagcaaatacactcaaagctcagcgttaacccaagtccagcgctctcagcttacacaggtggcagcccaatcttagtgctccactcaatccatgtctatgtatacttaattctctggaactaaaatggggaatagggccaggcaccatgactcaagcctgtaatcccagcactttgggaggccgaggtaagtggatcacctaaggtcaggagttcgagaccagcctggccaacatggtgaaaccccatctttactaaagatacaaaattagccaggtgtggtggcgggcgcctgtaatcccagctacttgggaggctaagggaggagaatcacttgaacccaggaggtggaaattgcagtgagccgagatcgagccattgcgctccagcctaggtgacaagagtgaaactctatcttaaaaaataaaaataaaataaaataaatttttttaaaggccggagggactaagatgggagtgggaaggagggaggttccagctcaatcctcaccagcagatattcctgcatcctccatacctgagccttcagaccaatgctccccaggcccaaaggggacctccctttcaagtataaggctcccctaattcctcaccttaggatctttaaggcagatgactgccttaaagaacatatattccttgcagcaagaaaacccaactcccaacaccccacagagtgtggtctggcctggacaagtctgccctaagaaaaaagcgacaaagacccttagtaattgaaacagactttaagtaccataaagatgagctttcaatcattaaaaaaaaaaacgtttactgagccaggcaccaggcctaggaacagagaggtgcatccaggctgccatgttaggtgggcctgggccccactgctggggtcagaaggtattagaaagggtctggtctgctgggcgcggtggctctcacctgtaatcccagcacttagggaggccgaggcaggcagatcacctgaggtctgcagctcgagaccagcctggccaacatggtgaaaccccgtctctactaaaaatacaaaaattagttgggcatagtgatgggcacctgtaatcccagccagctactcaggaggctgaggcaagagaattgcctgaacccaggaggcggaggttgcagtgagccaagaaagcgccactgcactccagcctggatgacagagtaagagtccatctcaaaaaaaaaataaaataaagaaagggtctggaagctctaggtgacctcatgctcacatctatacgcacaccttgatacattatttatggatcaggtggcacagcattgcaggcagcagcagggcaaagacagtgtggaatcagagtcgggcagcgttggatgggattccggttctgccaccactggctatgtgatggaaatctaccacatctctccaagccttgctgtcctgttttatcaaatgaggacattgtactgaatcacaaggtctgtacaatgcccgtgcatgtgcctggaatcttcccctctggtgtcccctcaatgcccatggtgcttcctgcacagggtgtaccatagttgcttgctgtctcctgctgaagcctgctgtgaagagggactgtgtggaggtaaggcagtgggcctctccctgtgaggtcctcagggggctgaaggcaaagtcagaagtaaaaaaagagaggggggacccatccccaaccagctgccatcccagggcactcagcctaaggccactgcatttgcatgctatttacataaatctgcatatacactgtattcccactctatcttgtaggcaattattgcatttaggcagctctgcattccaggaaggggcacaagcagagatgtgatcccctgaacttcatgacaagaaggtacccgaagctcctctcccacctccagcagcaaatgtctcccctggggctgtcctgtgttgtgtttgaaccctgctctggggctcctgcccgccacctgtagtggctaccatcagaagttcctgttaattggggatggcagggtcaccacatctctactcttctcccacttctcagcagttctaatttaaaccaggttagaatcttcccacttggtagtctggtagggaaattatagttaacaataatttattaataagttaaaatagccagaagagaagaactgtaatgttcccaacacacacaaaaaagataaatgtttgaagtgaagcattgaagtgatgctccaattatcctgacttgatcattacatattgcatgcaagtaccaaaagatcacatgtacccaaaaatatatacaactatgatatatcaattaattttttaattgattttttttcttattttcctgtgtagtggcagaatcacagttcactgcagcctcaacctcctggggttcaaacaatcctcccaactcaacctcctgagtagctgggactataggccctcacaaccatgcccagctaattttttgtgtgtttttgtagagatgggggtctcattttgttacccagctggcctgtaactcccgggctcaagggattctcttgcctcagccttccaaaatgatgagattacaagtgtgagccaccgactctggctgatatatcaattttttaaaaaaaaggttcttctccaggcttcaatgcgacttcacctataccaggtgtgagcggccctcatgtccaggtgggtggggccaggcatctgcagggacctcaggcttcttggattgtttgggatggaggcccaaggaggcggaaggtctggggtctcacccaagctggctctccccaaggaataagagtaatcacatgggcctggagcagaaaagggaagagaagggtccccaggaatgatgatttctggggcttcaggaaagagtgtggaactgaatggcagagctaagggaagactaggacttacaaccaccaaaatcaggccctccagctgggttcggggccaagggagagtgcagcctggcttctgccattctcctttcatccccctggagcttctgaaatccacagaattgtggtgaaggacaaggagccaagggctcacaggcgaggggaggcggggaattcaatgtgttcaaggcctacattgcacatctgcccttggtttactctatctaatgttcccatttgaccggtaagaaaactgaggctcaagattcaaacccaggtccttgtaacttcaaaaaccttgttctttctaggtgtcaaagtcaatagatgggccacggggctagcagtcacttctcttcctggacttggcagtcaagggataggacctgaactaaattgcctttctacagacttttcagccccaaccagaggcctgctgcaggcagttgtttgaactccttgattaaatgttcagcccctggcctgggcggcagactcccaacacacaagacttactgagtcccccaaatagacttcaaaacacagtcctgagactctgcctgccagttgcccattaagctcatcccagcctggcatcttcctttgttggtccactctccctatggctaccagggagaaagtcttttcagagatgaataagggcgtccagacaacagagatgtagtagtaggaggtggaaccaaaacagaaccaggggtctgaatcctggctctgccactttctgagatatgtcacatcagacaggtgacttaacctcctgtgtctcagtttccaaattcaaaaacggaaaatgtattatgacgatcagatgagttaatacagtcaaaatgcttcttagaacagcatctgggatgtagcaagcactcaaaaacatattagctattatgatcacacagagatgcatacctcctaaaatctttctgggctcccaccgccagcatgataaagcctcacttctcaccaaaggcaaatcaggcctctcatcatctgcctcctccctccccagccccatctcccgaagcgcctctcagggcacctctgcccttgcacaaggcagggcctgttcctagcaactctcagagtagcagtggaagtttccaacaccaagatctcagatacctcaagctggcaggagtgcgaggtccaacaagaagagattttatgaatcagccagccatgggggtggggcatggccaggtttcctgattcccagaccagggtgccaacaacacctactgttcatgtgagaaaaggagaaagaggaggaggggccactggcgtcttttgcccacctggggttcctcgctgtgggagagggggaggccagaggacagcaagttccctgagagcctctcagagaaaaggagggtttagatcatcacacaggataaggaagaagaccttttccaagggggtggagcagagcctgcctggaggtaaatttcctttgggaactgtcttcttcagtatttcccttcctctctgaacaccgtttcttttcctttcagattatctgacagccttttaatgaacacctactaagtgcccagcactttcctgggaacttaattcttacaacctgttaagaggtctatacactcattttacagtggaggaaatggagacttagcggagatgtgaaatttgaaccaggtccgtgtggtttcaaagcctgcgcatgttcctttaccctatggaagcctccagaagccaaggtctcatctcccctcttccctggaggccccaaacctgggtagctgtgcccctagacctttgagggagggtctcgagccattatctcccttaccttttcttgcagtgacttggcttctcccaggctgccgtaaccagcaaacgcctaaagatggctctgagacgatggccttctgtgtgtgtgtccatgaaatcaggtagtacaccacacagaaccagactctcagtgacatgcctgatattccacagacctgcatccgagctgtgcagatctcaggtagacacaaccagtgattggggatggctggtaggatcttgcctaggcaggccagggagctctgtgaggagtgtttgggagaaaatcccccaggttcatctccctgcatggactgcccctgctgggccctcacactaagctggtcctggtcctcagtactgagggatcaccacaggtgtgtaggccacagctcacaggagccctccacctagcgggtcaaaggtagtggaagatctgagagtcaggcagacctggtccaaatcccggatctgccatttcctcactgcatgatactaaaaaagtcacttcttcatctgaaaaatggggctgatcccacccattctgcagggtctcagtggggattgaagggggctgacctcaatgtagcacgatgcctgccccaggtgggacttcagcaaactctctttcctttcccttccctccccttgaaggccaagaccaaggaaacagaattgggcatgccagaacatatcccagccctgtggggtgtatttacctccacttctgatcacagtgtgtttgcgtatgaccttgaggccagtgacttttacaccttgcctatgctgggctgggctgtgacttggctgtgacttgcctgccctgaagccaagcctcagctgacatgtgtctttccaccacccctgctgtgctgagtgagcaccagccacctgtcccattgttcctgccccaggctgtgtgtggagagtggtctgacagggcagacaacaccctacactccagatgtgttgaggtgagctctgggggtcaggaaagaagcagggaggggcacagtgtgaacatcctgtgcagatttccccccaactggccccccagctggttgggttttcttttctcagtaactcctggcagaagtccaaggaggcctccttctcccaaaccctctgtccttggctcaaaaatccagcagaggacgtgccacgaccacgaaaggtgacagcagtcaaggggaaagaggactcccttactggagtgcacagaagtttcctatttattttgcttacgcggttgactttttccaaaggctgcttgggacggcttacctcgaaaagcttagcaataaggctagtagtaaatcagacaaaaaaatcatggactgggggaaagaaagtagcatttaaatatatttaataaggacgatatagtcgtgaacatgtgcctgagtatcctggctaccacagtaaaaggggaaatatgatcagttatgggaaaagagtaaaaatattaatcatgattttttttttcttggagctttgtgtaataggcactgatgttgaaaagggtagagttatttttccatatttgtagaaattcagaagattatcagactatttgcctcacaaggactttttagcaagcaaaaagtatgatattgtcatgcaccctggtgaggggaagtgtcatccagttctaggtttctgtgaatctaccttgatgcaggtgttcagggagtagtgagtttcctcagttgtaatgtagaccagcgcttctcaaacttaaacacgtatacaacctcctagggggtcttgttaaaatgccattctaatttgggagctttggggtggcctgacactctgccattctaaaaccctcccaggccatcatgatcaccaccttccgagcagcaaggggttaaaacccaggcatctcccatgatctagtagtgagtgcctttctaatcgcccccagctgaaaggcaatgcaatgttatcatagacgggcaagaagaatctcttccttggctctgtaaatgcttccgggatgtgctgggtcagcctccctccattctggttggcgatcttggtctagtcacaagccaagaccgttgatcatacacaaaagtttaagtacctactacgcgccaggaagctccctcagaaagatggttggtcctgctcccccacccaccctccaccctggcatcttttatccactcttccttcagcaactgcctttatcaatgctcccttgagccaaaataaacaaataaaaagcaacttcctttggaagctactccatactcttgcgctggccacagttacatctccctgcagatacacagattctcatgacacagagctcagacagtgcttcctctctggagggctcatgtttcgagagaagactggaaggaggacaaccttatgctccagtagaattccaaattctggggtgcagggacatttcgtgtgtgtatgtggaggggggggtgtcctaatgccctgtaagccacactttataatgctgtacaatattgggtcaccatcatgggccatggaaagcagaatctgtgaggttcagtcccaggctccagcacccctttgtcagctggcctccccgctcagtgccctctcccactacctgccctgcctcagctctgactgctgctgatgtgcctccacccatcaggtcattgtgatggcaaagacaatgcctccatttgccctgaatgcagccttacataggaaagagcttcctgtgctccagaacttggaggagtcctgcagcacctggtctagtggggagagcatcagtggggagcggcacctttcctgcagccacggcccactgggcaggaccagatccaagtggcctggcttctggctagggtgtcagagtacattggggcatacagcttcccgtggccatctcgttttcatggaggatttattttgtgaagtggacctgctctgtcccatcctataaatgctggattaatccaagaagctgagggtgagcaacatccctctccttcctgatcaaataagaaataaatctagattttctcctggatgctattctcttcttagccaaacagctaaccttgtcttcaaggaaaaagggcaatcccaggggctctgtaaattgaatattcagcaactccaggggcagacatccctaagtgcataagtggagcacccctctgaatcagcatcacaccagctgggggcataaaaaggagagcagagtaggcaggagctgtggggacatttctgagatgctaaaagaggcaaggaagtgtgaggacgggtgtgcattcatgcaacaggtatttactgagcacctactatgagccaggcagtgctctatccattagagatacggaattgaataaaacataaaaatccctgcccaggaggagctcacatttctatgggagaagacagttactgaaaaacagtccaaaaaactctattttatagtgtgttcagaggtaatacaggctatggggaaaaaaagaaagcaagcaggtacatggatgaggaatgggggcgatcagcgtgggcctcacagaaatgatgacctttcagccaagacttggaggaggtagggagggtacctggggatgcctagggtccaggcaaggggaacggcaggggtcaggatcctgtcagattcatctgaggtgggactgaatgtgagcagagagagggagacagaggatgagggcagagagaaggccaatcacactgggccacggctattgcaaggactttacttttgctgggggagggggtagagttttgagcagaggagaaatgcgatctggcaaacatcttgcaaggatggctctgactgttctatccttggcagaggacaccagagtctggcccatagaaggtgctcaacaaagctcagcacattgcactcctccctggagtaggaaagggctcgtctgctttgggaaatttggctctcaagcttctggaaacacctgaccccatgccaggtgagacaagctcacactcaaggcccccttagtccaacggtcagtggacagaacaagcaccaaaggtcaacggcgtgggcggtggcctctctgccaaagtgtgtgggcccaagtagggtattcacctccctccctgtctgtgagaaatttcagacccctctactttttccagcaggagttcatgttccttttaagataatcctctatagacctctggcagcccagccttcaaaatgtctccccctgtctctcttactcatggagagccccgcttccccgcctcaaaaaaaagtcttcctctctcttactcatgcatggagagccccacaaaaagacactggttcccagggattcttccaaagatgctggaagacctctcagtgtttgcatttcccaagctcggtttccttgtgctgtccttgggcattaaaggaaggctattcagccatccccatcagcaggagcaggcatcaaaagaacagctactgcagggggagcatattaggggcaccagagggggctaggaactgggtcttgtcttctcaaagtttacaacccactgcaggggccactcaccagccctggcaccctaagggcaggaacctccattgggcacgtgtgtctccagcactgggagcactgtctggcacctggggcactgtgagtgttgaatggataaaggacctgtaagaattgccataagaggtgttgccctggggcaaggtgggacaggaaagttcttgcaagagaagagaggctctggccttgggccctggctgagcagagagctgggaggtgggtattcgggtaggaggaaagctgtggagggggaaagcaaactgcctgcgggggtgggggggttctcggcgggagttgggggtggagggaaaggcgctttacagcagcactccctccacagggagaccagagctctgcctcttcctccccaccgtgccctttggggtccagttgtggggccttccttgaggcccagctcctgcctctggcctccggaggcttatctccccaacagaggctggctccccagaaaacaggattcttagacttcctaaaaataacctgaagctccttatcagaggctaatagtgtatgtctcccaggaatactctagaaagagtaccaggggtcaggaacccccactcagcctccacgaatctgagcggaaaagcccgggtctcctcacctgtaaagaaaataactgcagccggcaaatgtgaccacaggtgagaaggggcttcggaccagggcgcacacctctagagaggtgcccacagacagcctccccttacttacgccctgcttcgctgcagtgctgtgaagccatcggcattggaagtgcggagggcagcgggcagtgccccagggggaaggggggacgggtagggtttcagagcccagaaggcctagacagggaggcagctccgggagaaaggtacccgccatcccaccggtcttggaacctgaggactgctgggaagggtcaaaagtctgccctaagggtggacaagggacagagattggggggggaccacaaaggcctgggcgcaaccttgttgagagggaagggatccggacccaatctggagccctggtctcttcccacctgcaaccgtggctgccacttggcctcggttataaaatggacaaggattcgaggtgctcctcaggttaagagaggggggcctagaggtcaccctggcctcggtgccctccgtggccgggttaagaggaggtcccggagttctgctcacttcagccgtgtgccgggcactgcaaatcaggaagtgttggcgccggctggcgacctcccgcctggggccaggggaggagggtggttggacgctgccaccgctgccggggctgtgcagggctgggcggggagcgaggacccggcggctcctgattgcggccccgggggaggtggccgagccggataagctgcggcgggctggagggcggccacctcccctgcaggtccggccctcccgggcgggtggggcgcgggggaggaggagcctcgggccgagccaccgccttcgccgcggaccttcagctgccgcggtcgctccgagcggcgggccgcagaggtgagtgtaccctcccccggtctccgcggggctgcgtgctgcgcccggtccccgagacgcccgcccggttgcaccctgcgccgtcgctgcgcggacctcgggtgccgccacacgtctggaggcgacttctgtcccctgggaccgagccacgtgcgcccggcggcagagaaaccgggttccggggcccccaccccgtgtgccttccttccctaggcgtggaagccgcttgcgccgcgcaggttaggcagggccggcggcgacagtggcggggagcaggctccggagccccgggtgcagatgtgggcgcccctccggatgaccccggctgagtccacaggtcccgtgtgccccacggctggggctctggccagcggtcccaaaggagggctggtagctgtgggcggggatcttccaggctcttcctcgccagacctcccagtcccttcgcagccgaacagggcagggacaggggcaggtgcaggggctgggcaggggcgcccgaccttggtccccaggaggaggctggcctcaccagtgcccgcctctgcccgcggagcccttgcctggcaaagttccccctcctgcccggcaaagctccccctcctgcccacttccccgaaatttggggacaggctgcccggagaggtgtggttggaactttggtcactctgggtaaatgaaggggagggtgtgcccagataatatgcggtttggggaccagaaagccggatccgtggggagaagcaggaaggtcccgctggagccctggcagagggcacagagaggcctcccaaatatccagggccacctcagagatgggctccagaagtggggattctggaaatgcccccagggaggtatttctgtccatcagagtgcagccataatataattaacagcagatattcccatttatgtgtgccaggcatcttgctaagggctttgagcacaatatcccagttaatcctcacaacaggaaggcaccattatctcttgttaaaaatggggaaacagcctcagagagtccacgtattttgcaaggatacacagctaggaagttgcagaagtagaatttgaacccagagttatctgacttggagcccagaggaacctaccactggggcgaaacaggctgtgtcctcacggggattctggcagccccaagaatacagtgctgagagccagaaggtccacaaaaccaaagggtctcacctgggtgtgtcctctcagtcacctgcaaggcttgttaaaaatatagatccccaagtcccatccgaagctgactgaatcagctaggggcacctggaggacccagactatgtcttactcatctctgtccctggtggcctgcagaggggctggcaaggagcaggtgcttagtgaatgtctaatgggtaagtgggcggggtggggtagatggtgtcactagtgtcctgaagggggcttaaatggagcaggtggcctcagccaggcaatccggcagcaggtatggaagtgacacagtctaatgctggcctcagagccagcagggagctcaggctgggccacttgtccacctgggggttgaacagcctgccccaggtttgctgaggtccccaggaccaggccaggtcaagctgggatcacacatgttccctctccagcctagagatgtcaaacaggtagattcctctcccattcatatctcctatccttggcccacagcccttcccttcttggacttatcagagaccaaggtgctgggcagggcttcaggtggttaaaaagtgaaagttcttgagtgaagtccaaaggcgcacacctgagagctgagtgggcaaaaggtcgctggctgagtgctggggatagtctggctttggagtcagatggacgagtccaaatctcagctccttaccccgtaacatgaagccctcagctctctgaacctctgtttatttgcaaaaccttgccaagggcttcaaacaggatatcctcataaaacaagtatgcctatttgggggactaagtgtgtgaagtgctgagttttaggagtgcagtaaatatcggatccctccttcttcacctcaggtttgcgagttaaccagtgaggggccttccaggcctagtgctcttgtagaccgaaccactagctttcctagagaaggcaactgaggcctagagagtggcctccaggttccgtgctctgtggcagagctggccttggctccaagtcccagggttcccagctcagtctcagctgatgcctcccagcctggcggtctacgctgcctgctacattccctgtccagctacaaatgaacaactgagagggctggggtgagattcaggcaggctggggcaagggaaggaagcaggtcatttggggagtcgggctctctggtcttccagctgaggctggatccactctttcctgtccacccacggtgcgtagtggccaggtcttcctgagctgagcaggttgagcactatgaaactgctttctagcatctagaccagggagttgcctcttgatttaaaatgaggatgtttaccagtttaagtagttattaaaacctaaggccaagttggtgcctttcctccctctaggagttggaagggtgtgtgcttttttctgctcctaccccacccctgccaaaccatcccaatttgagaggctgtggagattacagaacttggagaggagaggggcagggatcggggttggagactagatggttttcaaacattgggctacagaagtccctcttcctcccatctgtctcctgcagctgggtccacccacctgtccaacacagcctcccacaggtcccgggcctctttgtgctccaaccgtcacatccttggcatgccacacaccctcaccatcctgcctgaaatgccagtcctcatcactgacattcagcctcccatgtgccagcatccgggccaaacactttatctacaggagctcatttgtacctggagactcttaggaggtcgataatgttattctcccaaccatgtgactttccaactccatatggaccccaacctctttttttccacccctaacctgagactcctagccactaactaactgccacattctcctgtccaaattctgcctcatcggttattcattcaacaaacacttaccgagcactaactttgagccaggcactctaagaccagctgagtcccacttctggcaggatgagtgttgttgtcatcaaccaccccacaaacacccgtgccctctgcgtacttgcgaatctgagtgaggagacacggtggcagtaaggctcaggacctatagctcatgtgagtgagcaaggatgcaaaaccaaagacacccgctcccctcacccactgagctggagggatcaaccactgtgcctgccatcagggctgatgggcgactgcttgtggatcacaaactcttgcgttgtcacttcaacccaggggcagagtgtgttcctcttcaggtgtggcctggtctcgtgagccctgcactgaatgggccaggcgttcattgcttcagcaccgtaagaacactgtcaccggagaattgttcgatactcaactcttcccagtcagtctgtcttctgcatgcccacagttaccgggcagcgtgggcaacaagtgagcactgattgttccgcaatcaggctgtgacttactccatctctgccttctgcttaatgagagccaggtgggagttcagcaaagatcacatgtttaagccttgttattaaacacgttaaaaaaatactgccccaagattataaagccgacttcggtacatataaagagatttggggtcggtgaatgggcccactaagctttattttgtggtgtcagctgtcttggttcatgtgtagacagcagggctcccaaagccagcatgctggggtctgtgagggaacaagcttcccatttcttgtatttatttatttatttatttatttatttattttttggagacagaatctcgctctgtcacctaggttggagtgcagtggtgccatcttggctcactgcaacctctgcctcccgatttcaagcaattctcctgcttcagcctcctgagcagctgggattacaggtgtgctccaccacacccagctaatttttgtattattagtagagatggggttttgccatgttggccaggctggttttgaactcctgacctcaggtgatccacctgcctcggcctcccaaagtgctgggattacaggtgtgagtcaccacagccggctgcttcccatttctataagcatccgactggtggggtggccagtaggctgtggggagtggtgcgtgattttcctggtgggcttgagagagtaacagtatacccccagtcagggcaagtgtctctctgaaggtgaactctgggtgcacagctagaggatacagtggctgtgagctgggccctccaggtgaggtctggcctgcccattgccacagtgggcttgctggcatccaggggggctttctgggtgactgcccaggcctgcacatgagtcagtagcacgctgaatgcagtggagtttgaggggacccaccactgtccatcaaatcgccccaacctgggtgaaaggccagagcagctgcgggaagtgacaccaggccctctggagatgaccctctggcccttggagatgtctctggactggccagaaagaaggtggtgcctggaactggaagggaaggaagcaagacagtgctgggcccagcagccagaaatcagggtgatgggtgggtggaggggcaggcatctgggaaagccctgccctcctggtgttcccagatctgaaggctggaggctggaaaccttgtgtcacctgtgctggtgtgagttccattggggctgccgcttaccctcccatgacctttaacatctggcatggcttgtggggggtgctgagaggaggagaagtctcaaggctgagggagctcagggtagagccaaggtctcagtgagaaccccagggggcagctgtcacagctgacctggagccctccaggaacgcgtactctccagctcctgtactctcaaaagcaacagaaaggtttgctttcctcaggtgcggtttcctcacagtgcagcttctccaaagagatctggagaccccccagcagctttgacaggaaaatagagtcctatccaccccacaccaacacactgaagtagagactctgggcctggggctgcccagaaacccacccacttaaactgcccgggcgattctggtgttcacaaatatttgaaagcactgcctgacacctgctggctcaacaggtgtccacctaggctggagtacagtggtgccatctcggctcactgcaacctctgcctcccaatttcaagcaattctcgtgcctcagcctcccgagcagctgggattacaggtgtgctccaccacacccagctaatttttgtattattagttcacctaagcagagagcgatgtgaggggctgtgaggatgtgcaggggtggagggcacttaggtccctccctcagctgaccatagagcacccagagtggcagaggggccacgtgcagtgagtggccacagcagagcaaggaaaggtgcacacaagaacagtcctaaggaagacatgtgtttaggggtttcatattcactgtttaatcatcaggacagagagggaaagcaacctacccaacgtcacacagctagcacatggctggaccggaattcacacacatctctgtccagctaggtcccccaactaacacagctgcctgtctgaattcaaagccgggagagattgtcaggggtgggcgttgggggagatggcaggtgccttaagtgcccaaggccttgggctggatctttaaggaagggggattgggatgggccaggaggttgtcaagattggagatggaaagaatgccattggctgtggtggggggtggagtgggcagggactggaaggaggggtagtggaagtcagtccctgcctacaggcctgtgttagactccgcggagattcaggaatctagccctgcccacaggggctttcactccagtagaagagaggagacccagggggccacagcagtcagagaacaatgaggccttggcttggtgtttggtgatgataggggaaacctgcccagaacctgtttatcagatgctcagggatctgaaaaacaggttctgcatgtgggcttgagataaagatgcccgggatacttagatgtgccctattccaagatcgctaggttccaaacaagttgaaaacaatagaatacacttatccctaagaatgggcttgcacataattcacttcaacaatgcaaaagcttccaaacctccagcctcctttgggctgccggtgtggcctccaaggtccactgaaaagccatccccagccagcagaaacatgcctgtgtcttcttcagcccccgtccatgctttgccctggagacaagtgctgtcatctgtttgtgggtctgtctgtctgtctgtctgtccgtctttctccccactgtgagcactgacaagcaagtaaagatgacaatgaagcctttgtgggcaccaaatgctgacagcattatcactaagccagtggcagccatggtgactgcaataaatttcccccacagaaattgataaattgccactttctgcagccattatgtttgcctttgtatcccgaagagtaaacgcctctctccctttctctctctcgctcacgtgtgtgtgcgcgcgttagatgacatttattcattttatgcatcctgggttctactggtcgtcccacctcagttcctgtagcaaagagacttgagtctgagccactaattatcacccgtgaggtttcctccccgagcaggaagcagcaggccagagctgcgctctctcagtgcactctccaaccaagcatcagtcaccactcccggtccagcccctgtggccaagagctggcgtgcaggctgcgggaggcagctggctgtgcaagaccctggcagggccctcgcctcctgagaaaccgagagtcagaaccaaagccaggctgtcctggttggagactgagccagaaagggtggctcacctcacggtgaggctgtcgagtgacctgagagcctcagaccctcacgtcagccggatgtcgcaccagcctgctgttggtaagtctggctaggacgcagatccaagggggcatgggtcgtgtgcaaacccctcccaccagctccctcaccacctgacccctgccctgctggagggtgccccttgaatctccaggaggtccgaggaactcgacacctcccagagatctgtactgccttggaacaggctgcaaaaagtgcagtatgaaatgggaaatatgtatcagctgcacgttttcatggccccctttggaggaaagggagtccaggttattaagtcaggaggcttttaaatttcaggggttcaaggatttatcacacacagttgcgtgtgtgtgtgtgtgtgtgtgtgtgtgtgtgtgtgtgtgtgtttaaggatgttaatgtttatgatgcatttctgtggaatgcaggcccattgggaattgtaaatttttcttattggtaaccatttctgcattggtttagcacggaactgctgctgcaagtaaatgaaccagatttttctggaatgattcgaaaatctagctgtttgtaaaaagctctctaaattattcattcacttttattttgcacagttgtgtgacggtaaactcttttccatcagacgaaacttgcgcgcacgtgtatttctgcggggattggctcgagtgtgcttgagttttctgtattctcttttgtaggaacttctcagccttccttttaaaaagataataattttttaatgatgtctaaataatgattccaggcagtattacaagaaatataacagtaaaacgtttgtaagagtccatcagaaagggaaaatgcccacagagaaaaatagaaatctaccaattagatgtgtgtggctataagtgagcttttaaaccatgatttgagactggcatcctctggcctctctgccctcaggtcagctgtgggaggctaggggtgggatagcaaaagggacctgggatcaggtggggcttcctggaccccaggcccagtaagaatgaagcctaggggttctaggatagggggcagccctggatttgcagaaacagcctgagaatgaggggctggaaagtatgagttggagcagctcagtgaggagacagaaaactgaagcagaagaaagcagaagacaccagagtctggtgctgactgggtgactggctgttactcacatcctgagcaatgagccacagaccctccccaccccagaggcagcttgaatgcagggactggatgggcccagggcacagtggatccggggcaggcaagagccaactcggggcccagaggaggacctttaacctgactgggcctcagttttctcttctgcaaagtagaaggaattatagccttctgcaggtcaaaccctgtgcccacatccacaggcacgatccccgacttcgcctcgctggcacttctctgcatttgactcctggttattgcagctgtgcccatgccaggcatttgttaggtgctgggatgtgcctgccccgctcctctctgttggggaagacggaattcatgctgtaggggaggcccggagtttctacatggggcagtcacagggtgggggacttccctgaaaacttcttatttgcataaaatgtgcgctgcttttgacttatattgtaaatttcagatccataaaatgggaaagactttgcagaagctgttattcactttacctgactgggagatgtagattgttcatatccaagggtgttagttttaatactaacattagcatgtggggaagggtgatcgagatggtggggagtagggccccccaccacctacccctgggacctctaagggaaagtacacaccaccgtatgctagttgcatcctgttacctgtgctgcagagccaaggggccagaggcagggaaattaattctgccttgtaaaggcactgggccttcctgttgaattttgctacttgcggggaaggcacttcccaggcaaaaagattaagcctataacctaagcagaaagcaagcaagagctttgggtgggaagtggccagagacatagatcaggcagacccaggtttggaagggccaaggagacagcacatctgccaatggcagcagctgggcctcacctgccctgacacccccacctaatggggactgggcagaaccctccaaatggttgccatgccctggcccaggagcctccaaaaaccctgaagccattgactcagcatccctgaagcctccttccatgagtacccccataccaggctgcacctcaatgcaccttagatctggtgtttgattttcactttgaccagatctgcagcagtctctttgttaatatttatttccagtgtttacattcatctttgcataagtcctcagtgttactaagtttatcaaatagcgaaatgcttggccaggtctaacaatgtcaccaagctgcagaagggattagaaactcatagtattattatttcatcaccactgagtacaagagagccccagctaggttctggggagaacagctatgactcacaaatactcccctcccaactcacaaatactccccttagaatctagtccttaaatcattcaaaccacaaaaatgtattgagcacttactgtgtgctaggccccggtaggtgttagaggcacagaaatgagttttttattggcccccatccaatgaggtgacagacaagagaaccagtgtgtgcagccaatgttaatgtgacaaaaagtgcagagttagtcccagagcgtgaagtcctccctgtcctaggcccaggtggaggccaggagctgctccccgggctgcctggaccagacttttgagctcttgggaccctggcgaacatcaaagctgttgcaaaagtcagagctcttgcaaaagttggagctggcccatggcaggtggttgagaagtggctacatgactcttcccatgcagagcggttagagggtggaggccgatttccagtatccctgggaatccaaagaaagaggccactgaggggcaggggagggcccctccgaggcttccagaaaaaggcagtgcagcagctgggcaaaccaggataaggcatgcatccctaagcccaaaaatcaggtgatgtgaggcaggccgagctccatggcagacatgctgagctgcctctgtgtgtcttggctaactctgccctttcctaaccccacggctcaccctgcccaggagcagggatgaggcccatccctgattcagcaaatcctgtgccataacaaggggaggggtgggggcagccacagcccacaagacccaccagtcccagggtttgtgcagctccaggtctccctggaaagcaagaaaccacccagctccagacgctgaggctgcagattcagctgccactgcctgatggcctccaacctggctggattaactggcccatagggagctggctgcccccacaagccagctccctggcctccctgggcaaactaaagccctcctccctggctccatagcgcccggtcatcccctcattccagcactttgcttcttcctgcattgtgtagctggcctttctcctaagagccttaaaagggcaggtcatctttggccacctgcatggccccggcgccttgcttcatgctgggtacacagcaggtactctgggaataacaaataagagaatttgtcagaggaggtttggcaggatggactattgcatagaactgcacagactgtgcactgcacgcctccagagggcactctccacagggattactatgtaaatggcacctcctaaagttgtgcagcatagggaccctagggcttgagcatgccccacccgcctccgctgcttcaagcccctggccttggtgctgactgttgggctttcctcttccccttcccaaccagcactgaccatcatccttcccatacgctggcgatgggtagtaatgaactgtcccagtgagcagttctcccagactgggctgtcaaagcatgctagtgagagattcctggatggcaggcaggccatctggattcttgctcagccaggaaactctgtcctctgggaatatcctcccagtcagggtgttagggggctcctggatccccaagatcttcatggtcccctagaactgctcctctccccagcctgcagtgggcctaatgacccacatgtagccagggagtggtccccatggggtttgttttattgactcagctaacaatgccacaatgactctgattgccttcttctcacctgagagacccagctctgggcagccaaggggtacgtagggacagagcatatgcatgtgaccctcaagatctagaggagctcagagacagcatggatactcgaagcccccgtgagcagggaggactttctggaccaagtgggctgggagctgggccaactgtaaagacccagggaaaaaaaaagtcaaaactgagttgctcttgcatgcaagtgagtggttagttcattgactccgtccacagccctttcctgagcagctactgcagtggttttcaaagtgtggtccccacaccagcagcaccagcaattctggatgtggagcccagcaatatgttttcatcagtcctccaggtgaccctgatgcagctcaagtttgacctgatgtgtcctaagtgctggggtttgcaaaagtgagttttacttagttctcaccttgaggagcttggagtgggggagaagatagacaggaaagaaaacagtactgtccagtgagacgtattgctctggagggccatggaggcagaggagcagttggtataggcttgatccttgcaagggtaagtggcctggagaccccagtggcatgactgtcctcggcagggaaccagatgttgggaccagcatcccctagagggctcaaggagcccagtgccgaccccagtgccccagtgcccagccaccagggctgtggccctgagcagcaggtcctcctactcttccctccaccccagatcttctgctctgctcttcccccttcttcttcccactcccaagtcccaaatagtagcaggtcttttcctccgattgtcttggggagaaatgggcatgtacctccaggccattatcttagagcacatttttagaagattttaaagtaaatttttccattaggcagagaacaccgtgtattgatttgggtgcatctatttctgctttacccagacaaaaagatttacaaaatatcctagagaggaatagaattaaaaatgagtaaatttaacaggctcagagcgtagcactttatttgtctcatataatccttgccacacctcattaaggtgaggaacatcagcccatagtgcagatgaggaaatggggctcaggaaggtgaagtgacttggccgggccgccagctagcaagtggtctggtgggatatgaaattccatccctctgctgggcccttgctggctgaccttgcagtttactaaggtgactgatcctgaataaatccagagtgttctaacgttttgctgagcccgtgtcctgttggctgcattgtggggagtggcctggcctggctagggaagctctcagtgaccaattgtggcttcagctgtgttatttagagaatcccaggggctgaaatgatcctctgcctggttagggaagggatgactttaggaggaagagggacctaatgccccacttagcagatctgcagctgtgtgcatgtttccacctgcacatccttctataaaccctcagccatgtgggtgagcagcagacggtgcctcccccactccccctggaacacatctgccctgttcactgacctgggagctggtggggtggacctcagaccccagcacttggcacacacacatcatcccctgggccctggaatggcttgcatggatggcagagaaatagctgtgtgagcaaaggccaagagaacagtttagcccaaggcaccatttttacaacaacaacaggtaatggtactgattatataagcctcacttgtccagaggtcacggggcttaggaacctcaattatgtggaaaacaaataggacataacttcagaaggttgcagagccactgtgctagtgtggtctttgtgttgctataaaggaatacctgaagctggggaatttgtaaagagaaaaggtgtttttcaaaaggaagacaaacatttgaatgctctggatttattcaggatcagtcaccttagtaacctgcaaggtcagccagcaagggcccagcagaggagtggattcatatcccacctgaccacttgctagctggtggcctgggcaagtcacttcaccttcctgagccccatttcttcatgtgcaccatgtgtgcagagatcacctggggagagaggaagtaagagagagagagagggtagggaggtggcaggctctttttaacaagctgctctcgaggaaactaatacagtgagaactaacagggaacttacttgctcctctgccctccccccagccagggagggaatccacccccatgacccaaacacctcttattaggtgcacctccagtattggagatcagaattcaacatgaggtttttgggggacaaacctccaaaccacagcagccaccaacattggtgttacaaaatccatccaatgacataggctcatggagcgcgcttaagctgtcatccaaaacatgtttactcttggtttacacacactagtagcagtctgccatacctcaatttccttttttattttaggtaacaactttttaaataaccttactgagctatgcctcacatcaccacgcaattcacccattcaaatggtgagttgcatggtgtacccgtgaggatcactccccattttctccaaatcccccattctggccaccactaatctactttctgtctctattgatttgtttattctagacatttatataagtgtaattatgtaatgtctggccttttgtaattggcttctttcccttaatgctttcaaggttcacccctgttggatgcatacttagttctctttatggctgaatattaatccaccggatggataggccacattttgtttaaccattcctcagttgatggacgtttgaattgtttccactttttgggtattatgaataatgggtgaaaatgtttttgtgtggatatacattcttccttctcttggatatatagctaggagtggagttgctgggtcatatagaaactgtttaacgttttgaagaattcccaggctgttctccacggtgcctgcaccattttacaatcccagcagcagtgtaggagggttctgatttctccacatcctccctgtcttttaaaaaaatatatcattgtagccatcctagtgggtgtgaagtggtatctcattgtggttttcatttgcatttccctgagggctaatgatgctgaacatcttttcatgtgcatttgtatatcttttttggagaagtgtcttttccagtcttttgcccactttatgtaggttgtttgtctttttaataccgaggtgtaagagttcttcagatatttagaaacaggtccccttatcagttatatgatttcctgaacttttctccattctgagttggctttgcactttctcgatgctgtgctctttcgatttcgtcagcagcgaattagcaggacctggggggctttaagaggtgagcaggcagacagacagcgcccacaaagtcctgaggatgagagaagaggcagagcttacacaaaggctctgtgaaaactgaagatggaccccaggcaggttggggtgatccctggggtgctggatgaggatctgattttgtactgcattttggaaggatttctgcccccagaggctacataggctccagtggtgtgtgcagtcaggcagagagatgcacagaacctctacagtgtgccaggcatggtgtggggttctgggtgcatgtggaacaacacacagcaatcttgccctcccaaagctcagggaagtgggggcggggggtggacaacgagcacatgagaaaaagaccacaggggaagaactcccagaaacatcccagccacagcgcaatgccccaggtgtatatggcccttggctacttcatgcccttgagggaaaatcggtattttcattttacacttgatgaaacaggttagagaaaggactggcgtgtccagtacctcccagtgagaagatactcagagtagaccccagatccaggtccttcctctttccctctctacacgttttcagtctagaaaagagttctgtgcgagaatttttttgtttttgtttgtttgtttgagacggagtctcactctgtcgcccaggctggagtgcagtggcgcgatctcggctcactgcaacctccgcctcccaggttcaagcgattctcctgcttcagcctccggagtagctgggattacaggcacgtgccaacacacccaggtaatttttgtatttttagtagagatggggtatcaccatgttggccaggctgtgtgcaagaatttatgttttcacttgtggcttgctgagccctggtgctggctgtgggcacatactcgttcacccagggacatgcactgttgatgggtccttgcactctgggctatcttgggccccgtttgctcaatgcaggcccctctttctgaagcgcttttcttgggctcactctcaattcgtcaaagttcggctcaggcccacctcctgcaggaatcctcccagtccccgctgtccgaagtcagtacccttgtcaggagaccttaaacactttagtccactgcctggccctcccatggggcaaaggtctccagggtgtgtgtgcacttccttggggcttcttaaggctcattccctgggattagggaggacatacagagcctccttgcacctacatttgtttacatattacttataccacatacaacgttatagggcactgctgcataagtgtacattttgaatgtattttacttataaagaaatagagcagtaataggaacatgtgcccctaaatgtaattttactgaccagacaaaaaaaattttttttaattgaaaataagtagctggacgtgatagcatgcgcctgtagtcccagctactcagactcagaagtctgagacaagagaattgcttgaacctgggaggcagaggttgcagtgagtcgagatcgcgccactgcactccaggctggcgacacagcgagactccgtctcaataaataaataactaataaataaacaaaaatttaaaaaaattgaagataactgtgggctgcttgaggacaagaactgggttattttctctctctttgctcagtcagtccccacatgggaggcgtgcgaggcaccccgttcttggcctgcaggccgcccccactccatctggctgcagggtgccacagccagcctctgattgccagtggccccagcacctcaatccataccgacctcacagcaaaccctccgtggccacagcctgccctggccactcatgtctgaaaggtgatcggtgtccatgaagaagaggccatggggctgtggatgtcatgtggccaactgttagtggcctgcgcaagagcagacaggagctggcaggcaggcccatgggccaggcgtggctcccagccataaacacaggctgtagccctgccgggaagaaggaggggacagacaagcctgggcagagacccagaggggaccaggacacccgatcctccatggcagttctgtccacagcttgggaggagaggcaggcaggtctgcaggcaggagacaggcctcggcccacagagctgcaatctgatcccatctgctccctctccagttctataaatagaacccccagggagccagggcccagacagggttagtcacaccctcacctcaggccgcagggacaggcctctgggacttccaagacatgtaccacctgtcctgcccccaacccctgccagccttcagcaggagggttcatgctgagcatcaggagggggcagaagaattgaacgtggccactgtccatgaagaagttagagaagagtacacacaacacacatgcacacaagaataggacacgtgcacacacacacacacatgcatacaagattaggaaacacaagtgcacacacaaccagtcagaaatgagctctgagcttttacacatattaactcatttcatcctcaaacagtctttagagaggtataattagtgttcccgttttaaaggtgagggaactaaggcacagaggagctatgtaactcacccaaaggcacccagctactggcatgtgaggccatgatttctccccagcagccccacccacagcccgtgcctttaaccacctgcctgacccctcccagccaagtggctccgtccctacatcagaaaggtcaaggaagaggccgcctagggagcagtacaaggcagcattacaacctggcattttcctgttgcagactgcattttggtggcaagagaagctttggaggtgcttaacgtttagtttgcatttatcctaccgctgaaaagcagggacccttaggaccataagtctaggacttttcatagcaccctagactgctggggatgaaaggggcttggagctgagctggtgcagcagctcctgttagagaagccacccagagaggcccagggacctcctatgtcccagcgccagttaggggcagacgtgggaccagctcccctcggctgcagcggcctccatatcagctccctggaagggagcagatgccaagtcaggttctgagcaacaccctcaaggcccagccctggcaatcccttcccacctgtgattaggtctggggttcaacaaaaataggactcaaaatctggatcttctcgccttgcttcactttgggctggttcagttttgtttcgaggtaaatcattcgaggtttttcagggatcctcagatgcatgtctgcgtgaagccgtcctggcttccccttcggctgccccctgggcaagagcctgggccatacctgtgccattcctgagctctgtctctagctgccctatgtccagtcattggcgatgggtcttattcaagccccctttcccccaacccacactagattgtaagccctcagggtggggaccgcttctcactcatccttatatgcttaggagtgttcagcacaaagtcactcaggagatggcaggcagcggggagagggcagagacagaggagaggcttctgtccagagggttcctacttaggatcatccaaatcatcagcaaatgttggaaagatggtctgggggcctcaggctggctactcttcctccccatcagggcaaaactccccgggggcttgtgtctccctgtggttggggacaggcctgggtgtatctgtcccagccttgactgaccacaaggggagacttggccctcccgaccacaggagaagagggatttgaattcacctcggcccaaattgcagatgctggcctgcgagccctgaggccatctgcaaggctcctgggctgacataactgtcacccagctcttcatcctccttccccggggctgaaggggtgcctgcagggtcttctgccctccctccacctgggccctactgctggcacggagcttcatttctgttggcagccagtgtttgcttagtgattactgaatgccgtttgctgggtgcttatgatgtgctcagcactgttctaagtgctttacacaattgacttatttactcctacaacctgcccataagggaagtgctgccatggtctcccttttatagctgagaaaactgaggcacagataggtccatggtcacacagttaacataaggcagagtcaggaccagaatccaggcggactggctccagagcccggctcctggaccgagcagccttgagtagggacctggtgcagacccgcctgccgtggactcactataggatgggtcaggaggcccgttgtcacctacaaaatgtagcccaccacccaggaaacgctcccagaaccaggcagaaaatactgtctcctattcacagggtggctgtgcctggcgtgagtgtcttggcgggtgcctgcatgtctgtgcaggagtgccacccccatctgtggactcggaaagcatgatccccacaaaagcaacccctcagagtctcagctcagccaatccctgttatcctgtgacaggcagggccatgtttcaaaaccatgttaaagatgagcaagaggctaaggaagtttcccatgctggcagaggcaggacaggaacacatgtcccctgactcttgacattgagaatgcacccccttccacccacctggcctcttgtcccagccagtgtggtcccagctatgcagctctctgggacatctctggaaagctcattgttctctgtctgggcagtaagacggagtgaggctctgactgccgaggcagacagacctgcctcctgctcttcctcccctcaattatttgtgctgcctgaaacaagttcctgagcctccctgagcctcagttccctcctctgtaaagtgggaatgataactgaatctacttcctggggttcttgtgaggaccagaggcccatcacccgaggaaaggcctgacagcagcaatgggtgtatggtaggcatccattaataggaactgctgctgctgctgctgccttataaaaggaaaaatcacagtttcaaaaacctcttaagattgcatgatttgtcatctgagagcagagattgtgatctccattgacaaagatggaagctggggcccagggaggcacacagacttgtctgaagacacacagctgcttcatagcagagccgggcccagcctcctggttcccctgccccatctttatgtcagtccctggatccactccctcacctggtcaggtgtccagaggttccaggagagtgttgtggaaaagtaggaagaggcagacgcccatggagggtgttgaaagccaccccagctgctctgctgcttcagggcaggggatcttaagccctgtggaactgtaggctggtgaagcttgtggacttctgctcagaataatgttcttacatgcaccagacaaaatagcttagggttacaaatgaaatatattatatggaaacacagcgaaaatattttttaaaaaagactacagtagcattaggcatgcttctttattagcacattaaatgacaagctctagtggcagatctaatgtctaccataattttatttttacttttattattattattattattattattattattattattattattttgagatagtcttgctctgtcacccaaactggagttcagtggtgcgatcacttcagtcttcaactcctgggctcaagcgatcctcctgcctcaatgtcctgagtagctgggacgacaggcgtgtgccactgtgcctggctaattttttttttatttttttagagatgggacctccctgtgtggcccaggctggtcttgaactcctgacctcaagcgattctcctgccttgacctcctgaagtgctaagattacaggtgtgagccactgcacctggctagcatggttttaaatttgtgctcaatttaaatggtattaggataaattatttacaggtaaatttatgatatgatcttggtcagtattaatgaaaaattatgttgcctaccttcatacttaaaggaaatagcaaattttagttagaagttggtgacaattaatatataataatttttccatccaagtttactgtagaccgatggcccgagttcagggtttcctccacatccactcatatcttgggctcagcaaagaagatcacagggcacccactctatcttaggggcccattcacttgtctctcagtcctgccctcagagatcaaggtttggtgttcattcattcattcattcagcaaacactgagcactgcctccgtgccaggctatgatacacagtgaggtccaaggtcagatgagatgaggtcctgccctcaagctgttcaccatgcttcagggagagcataagaagaactgctccaagatgcaaaatgtagacaagcatagggacatgctcacccttcctggtcatcaaacacccagcgatggtgaggttggggttagatggtcactcctggctagtgacactgtccattgcctaatccttttgaaaagaagtatggcactgcatcacattggtcatggaaattccagagttccctttcccagcactctcactctgccagtttttcccgaggaaatcattcagcaggagggacaaagggtggagggcagaagtcatccctgctcttcctgagtctgtgctctccccagggaaggggctgtgttgtgttcagcttgttcccctggtgctgacacgtggtagcactcggtgaacacctgctgggtgaatgtctgactgccgtgtagttacgatgtataagaggagggaggtggcagaaggggcagtccaggagccctccactgaccaggttcctgacttctcttgcagccaccaaaatgccagaagagatggacaagccactgatcagcctccacctggtggacagcgatagtagccttgccaaggtccccgatgaggcccccaaagtgggcatcctgggtagcggggactttgcccgctccctggccacacgcctggtgggctctggcttcaaagtggtggtggggagccgcaaccccaaacgcacagccaggctgtttccctcagcggcccaagtgactttccaagaggaggcagtgagctccccggaggtcatctttgtggctgtgttccgggagcactactcttcactgtgcagtctcagtgaccagctggcgggcaagatcctggtggatgtgagcaaccctacagagcaagagcaccttcagcatcgtgagtccaatgctgagtacctggcctccctcttccccacttgcacagtggtcaaggccttcaatgtcatctctgcctggaccctgcaggctggcccaagggatggtaacaggcaggtaggttctgggggaataatacccatcgtaacaataaatataaatggctaattttcatcgagtgctgccagcatgctctttttgatatgttatcataactaatcctgcatcactgcaaaattccattttacagaacaggaaattgtggctcaaagacattaatgatcttactgaaaattccatagctggtaagttgggatttgaacccagttcctatggatctgaaacccatgttctttctgctatggtaacagggtttcctagagggcaaggaggaaaaggttgatggctttttgctatttggagccaagaaggagagagacttggcttttagaattggactcagaaggaggaaagtaacacacttatgtacccggccctgaggaaggcaggcatgtctcaaattttatcgtatttattctaagggcttccaagaactgatttctcagaaaagtcaacagcaggtttttctggggctagataatggctccacggaacagctagagctgcatacgtttcttgccacgaggagctcttaccaagacaaggtggtttttaatgccaccttggtttttaatgcattttcttggcgaaagaatgccagactccatgtgtgagaacttccaccagtgagtcgagcccaggtgccaggctctgtaggacagtttaaaggtgcccagccatacctgactacagcaccttaaatgcaggctgcatcatgggactcccaggtatggaccaggaggaacagtggagagtgttggatgcagggcctggagggaacggagcaagcaagctggctggaaaagacttggaggagggctttgacctgggcccacctcatgctgtcaggctcagggagcagggagtgcattccagtctgagggaacaatctgaaaaggcacagagataagaaacaatgctcctagacctcagttttctcacctgaaaaatgaagactatagtacctacctgcgggctcatggcaaggctgagagatcatatatgtaaagctcagcatagagtaggtgttcagtctgtgtttggagactaaactgaccaatgcagtgtcaacaaggcccacggggtgcttggggatctgaggactgcaatgtgcctgcagggggacctcgtgggtcagagcagcgggcttcaaggcggagagggcaggctggagctggcttgtagaaggccttatgtgctggctgagggctctactgagccgagggacatcatcaaggggggtgattctgaactgagggatggagctgcagccacagctgcccattctgcaccatacgcaaaactcttttgctgagcggctcccagggcaggccacggccttggagacagccatgtcgggggcccatggggactacctggatgagcattttgcttgtggttgctgatgtccacgagctgggcaagatgcttactgtttgcagcttccctccttgagtagttggcctggcttggcaggtgggccagaggctgggaggcctgggcagcagagaaaagacccctagcctgggagctagaaccccattggctcccacctgtaccattgactggcagctcacttgaacaagtccctgcccctctggccctcggtttcctcagctgctcagtgaggccctgcccactcctgtggcctgtgacgccgtctgactgccccacttttctcccgcaggtgcccatctgcggtgaccagccagaagccaagcgtgctgtctcggagatggcgctcgccatgggcttcatgcccgtggacatgggatccctggcgtcagcctgggaggtggaggccatgcccctgcgcctcctcccggcctggaaggtgcccaccctgctggccctggggctcttcgtctgcttctatgcctacaacttcgtccgggacgttctgcagccctatgtgcaggaaagccagaacaagttcttcaagctgcccgtgtccgtggtcaacaccacactgccgtgcgtggcctacgtgctgctgtcactcgtgtacttgcccggcgtgctggcggctgccctgcagctgcggcgcggcaccaagtaccagcgcttccccgactggctggaccactggctacagcaccgcaagcagatcgggctgctcagcttcttctgcgccgccctgcacgccctctacagcttctgcttgccgctgcgccgcgcccaccgctacgacctggtcaacctggcagtcaagcaggtacccaccccatgcccttcctccctctggcaactcagcacatgcttgtccagcacctcccccccccaccaaccaggtgcagccgatacccacgggtgcagccttaccaggtgccaactgcagattctgttccaatggggcagcacccacatcctcctgcccaagattttgagtatcagtgaacaaagcagatgaaaattcctgcccccatgggtacagcccagttggggcagacttaaacaaaatacagcagtgagtcatagtgggcgttgagaggatcaggagtgctaaggcgcaaaggaaagcaagcgtgggcggtgggggtgcaggtttgaagatggtccaggaggacttgcagagtgagtgctatgtgagcagagctggaaggaggggagaggggccggtggcctgctggggtgatgttgcaggccacggggcatagtccttgggcagaagtgtgcctggtacgttggaggagcagccaggaggcccatgtggctaaagctgaacaaggagggggtggcaggcgaggcctgagaggaagagcttcagccttaggctccgagtgaaatagggagctggtgaaggtttgaatttaggtgttgatcggcctgacattttaagtggttctctctggtttttggctgagaacagactgtaggaggtcaaaggcagctagctgctggggccagcgaggaagccgtgcatatgcccagatttgaggcagtggaggctggaggcagtgggagcagtggaggagcaaggagatcctgaaggcacatgtgaagctggagtgcatggaattgccgctggaatggccccagggttttgtgggtgatcagggatgactctggggtttgggtctgagcacctgaaagagagagttgccatcagctgagatggggaaggctgcaggagtgagcattccaggtttaggtttgttgttgttgttgttgttctttgtttgttttactgggggtggcactaatcattcatttagaaaattcatatggggcttggttgccagcagggttcctgggaagtggaggaccttcaggccacaccaggtggggacccaggcatcagctggcacctgtgcccacagaaacaggcccctgatgctccctcctggtccacaagctcatggggcttgattcttagtcccaagctcacatcttcagcaacactgtctttattttctccaaattaattccacctccagtgcccagccccggggctggttgatgcataggtcctgcccactccaggacgctagtcacccaacagggttcagcccaggtgctggtgagacacttctcatccacccctcattccagcctggcagcccctgcagctcctgctcctgctcagaatgcagggaccttggtgaggctgggccctcccctaaacacctagcacagggccatcccctccgagcatgggggattggcaggactgggaagcagggctgggcacctcctgagaaccacagacctcagcccctactcactgtcagggttgctgcttctctgccagcagctactcgactgccctcgtcctttgccacccacagataccggcatcattgcatctgtgggctcagaatttcctaagacaaaggaagaaccccagacttcaggcagaaaagcaagatgcttgatggaaatgagaggaagggaagaaatcagtcatgaacccttaacaagagctcgctgtgttccaggaaccattctaagagcttcgtacaccactgtgaattcatttaaccccatctcatccctgtaggccaggtgctattatatccttatttcatagatgagaaatctgaggcacagaggggtcaagtaacttggccaaagtcacacagtcagtaagtagcacagctgggattcagatccagctattttccagatccatgcttatacccacgctgctttctacaggcaaaccaaattcacaattgagtaaatgatttcagaactaggtcattcattcactcattcactgagcacctactctgccaggccctggggccacagaaaggagtaagaaagacacagaccatctggctcctcgcctcagctggtgtggggcggtgtggaggcctgaaggctagaggctggactcctgctctccagcatcgctgactggaacaaagatcttgaccttgagcaagtcacagcccctcttttgtcctcgaggccctgatgtgtagatcaagctctctcctagtgctaatgttttcaatgctctttgacgatattttcttactgacttctacaaagcaccagccccttggcaggaggctctgcggggctccctggggactagtacaatgttgctgcccaggacggggcagatctgcatggaggagcccactccctctcccctgagctggcaggtatttccagcagcagtgggaaagacggggacggtgcccactaccccccagggtaaaagacacaccactgctccctccctcccctgccacccttcaactgcctttgtctaaattctctgccctgccaaggctctccctgatattctgttaacccaattctgccctgatgcaaataccagggcacagaggggcgcatgcaaggtaattttccccgtgtcccaaaaatctttagggctgcaggccctggtgaggttgccaggcaaccatccctcccgcaggacgagatcagtcagagacaccgaaggcctctcatgcaaaggggcttaaacttgttctgcagagttcttaagggcaaaaccagggctgatgtggttatgtcggggtgggtggtcggagggaggcaggggcttgacacacagcagcccagatacggggaggtgcccaagcctgggcacatccaagcagattctggatggtccatggtaagccatgcccgcggttctgggcctgggcacctgaggctgttctgccttcagccccaaacagcagggactcctgggtggctcaggcttgatgtgtggcttctgctgccagcccatgggcacctcaccacagggtccccacatggccactcaccaccccaactgcccactaggcccttgggtcaagggagcacattagtctaggcacaagaaactcctccaagatttccttacagccctgagtctgcagattaggaggcaaaggtgtatgccagctgtagagaaaacctttgagaccatgtttacagacagtattattaccaacaataaccgatatttgtacagggcatttggatttgcttgtgtatttccctggaaatacacatttcaacatcaaactcaaagatcaagatcaaagaaatgtttaacctgcacactgttctcctgttaccttcaccccgttctcctattaccttcaccccaagctacatctgcacatggctggcacaggatggtctgaaactctcatctgaccatgtcactgctgaaaagatccagtgaccccagtgtccttgggataatggtagaaccctcactatgcagctagactccctccgcagcctcatctccccttctcctgtgacgtgtcaacacttgggtcccatactgtcctctaaatgggcagagctcattcctctcggcttggaacgcccaccccaggacaccttgacagcttgcaaactcctattcctccttcaaaaccctacctctgttaagctcccccagactatccccttacttccccatacctgcctctgtaatctgttgcattttgtaaatacttttttattatttttcttacacactacttcatacccgactcagaggtccatgactgggtcttctacaccttggggtccatggttcctaacacaggttcacagtgcatgctcactgagtgactgatttttaaattggccaccacagagccaatatcaggcatcttaagaggagtgcacgtagagcttggagcaagaactagtctaactgccttgctgccactaagaacaacagccatttattacattaagtgcccactgtgtgcctgttccatgctactcattccacaaacgtaatgcttttagtcctgcagtaacacagcaagatacaatgatgctatgagccaggaaagggactgcccagggtctcatgactgcccaccagtggcagagtcagcgtcagaatccaggcctatggtctctatgccccaaggttgcccttccacaccctgccaccccctcctcctctgagtctggcaggcagtgagttggtaccactctcttcccatccatcacccactatgaagacatggagaggggctaatgctgcagcctcactggtccttcagcgtctgacctgcaacctagagggaaatcccacacctgaatgctgtctttccatctcttgttgatgaacactctccttggccttctcaaggagaaggtggtgaacttgatcatctctaaacatagagacttacttactaggggagctgttacatctgcgttccagttagccatgtgtgccactggtcaccagggagctctgagcacaacactgtggtttaataggctggctgagaatctcatggactcctaaggctggcttaggttctgtcctcatgcgggtctttgccatccactgctccctagcccacctcatccctcccttcccagcgtcactgcagactggaaatgttagaggaaaaaaacaaggcattttacaagccaatgtggtatacgtgtgtgtaatcatccaaactctgacttgtgacattaactgcctgatcttcccgaggcgcagagaggaatgactaatgaagcagagttaaagaacaaacaacttccagcgaggagatgacttactcaattcatgctgtgaggtgtgagtaagacttctctgggcagaggtacggaggagacctctctgccctctgcacgcctttccagctaatggtgacttcaaccacagcaggcacataagcaaaatcaagagaatttcagacatgacctcatccaaacctcctaggtagagaaactgaggcccagagacagtcttgccagagtcacatgccactggtggtcaggacaagaggctgggtccctagactcctggtcccttagtggcttaaagtcaccaaagtgagccaggcctgccctaaaatagaacagagccaatcatttttcacagattgcaggaaaagcactcaatggatggcattttctctgtttctttaaactttctcttataagctgtacaaccattttgctcagcccagtggattcacttttaatattttatgtggtaactgagctcgtgtttttttttttctgctgaaattgtttctcaaaaaacctgtatgagtcattctgttttgagaggctgcctattaaagtgtagtcacatcttctaatggaaatactgttccttgttctgctgacattatagaataaaagtaccagcatttcacagtgtttcacagagaacaccttgacttccaggaactctgctacccggtctagttgcaccttgataaaagttcttcctcatgagggcactatgcgagtgagtgttggctgggctaggagggggctcagtggcaaggtggggagagaagggtcagaaatctccaggttcataggggctccccaatttatatgtcctggaacttggtgggggggcccagatctctgtgagggcagggtaagggcaggtatctttattttctgtagcccagggcctggcacacagtaggtgcttcgtaagtggtcactgaactgcttggtgaatggccgtcagtctctcttgcctggcaaagttgagccaaggctgtgtgcctcaggcagaagtagagaactgggtaggaagaggggaggctgagagctttgggtcggggagtgttgggggctcagtgtctgtatgtgtctttgtatgtcctgagtgtgagccagtgatgggggcagtgtggggctccaggaagacagagggctcagggtgagacaggctgggaagggaagcagccagtaaggtgtgagctcattggctgaggggattcagagctaggagagctcctctctcctgtcctgggattctggggccggaaaaccgtgagacggagccatgcttaagggctgtatttctggttgactccttactctgctgatgtgactgttctgattggctgccccggggcagatatgtgaaagccccaggagccccggtcattggcaccaggctgagtgggtcctgatcccagctctgcatctgttagctacatgtgaccttgggaaagtgatttgatctttgtgcctcatttcctccatctctaaaatgggctaatagtgatctctactttgtggggtggttttgaggattcgatgagttaatgcctgagaaacgcttatcacagagccaggtagcgtcaggtgcagtgtgagcgcccgccgtcttgtctttcttgtgtccttcatcattgccctcccggggcaccagcctttgccacagtgttcatggttttccttccatccatgtcaggtcttggccaacaagagccacctctgggtggaggaggaggtctggcggatggagatctacctctccctgggagtgctggccctcggcacgttgtccctgctggccgtgacctcactgccgtccattgcaaactcgctcaactggagggagttcagcttcgttcaggtaaagtagtctctagtctgccagccagcttcagcgtggccctggcccacctctcatgagtaagtgctctgcctttgagaactgcctgggctctgatcatctgcacagcaggaccactcggtgagatgccaggccttcctgacccagagggcccatccaagcctcttgataggggctgcctgctacaagcttaacctcccaacacatgaggaagccagacacaggcatgtgaaaaaattacagaccaggacactgagccctgggtccaccattctcaagcttctagaattcttgagctttccaaacaaaattgaatgacatgaagtcattggacattttgctggtctaggtaggagtcatcatctacctagacatccatctaggtaggagtctagggagtgagcgagcctagcccaccaccctccacccctcatctcaacctggctctgttgttccccttaaccccttgattcatgcagcgtggatgagacacaagcattaggaccccagtttataggggatgcgacacagctttttcccaaggatgccattcctatgaaaaaatccagaaaaaggaggtctgtcatcagatgaatttgagaaaagatttacttccccctaagagatcacaatgcactttggcacatcaaagattcgaataacatctacaacaaacaaacctttgttaatcagacttattggagtgttagcaccctccccctccttttttccaaataacagctatcagcactccacaagctgggcgctgtggcatgtggctgggatcccagctactaacagaggctgaggtgggagaatcacttgagcttgagcgattcttccaggagttagaagttcaagtgtccagcctggacatcatagcaagaccttgcctcttaaaaaaaaaaaaatactccacagaacatactctgggaaatgctgctagtggcaaaagtttctttttgcatttgttcattcaacaaacatttattgagcacttactgcaggccaggcatcctgctaggcctgtgatgcaatagagaacaagacaggcagagccatctttccatgataaaggaaacggaggcagggacattgtttacatttctttggcatctcccactgttctccacacagaatctgcggtcagtgttggcggattaactcctcccctcccatgagtgggagctgactaatgctgtatcctgccattgtaacagggcagagaacagccatcctatccacccccactatcaacagaaatctgtgcagggaggacgtggtacaggactcagggacagtgacagcgtaatgctcttacgtaatccaaattaattgctcctgaccagggatgcttcacatccattggaacaacacagagttgtagccgtgtgttttttaaagtgtgtctgcctctgaatctccctggctctgctcatctcagaaggtccctggaggagggctcatggctgacacatgttagcatacgagaaggggaagaattttatttatctgccactaatccccatgtgtctcatacccaaatctaggattttcctcttcccacctggagtatccggcgtgcatggtttatccacaattaaagcagactgcaggctgctcccctcaccagaaagccgctcccttaagcagccatagattacatgccctggctctgcctgccagagaaagccggtggatttgattttgttagcaagagctgactgagaccctagcagctgcattctactttacatattaaagagtggaaatacaaataggacagtccttttgccactgcaagagcagggggggttaggtgggtagaaaggcaggattttacttgtggaaatcagtgcctgccttccatccacaaaaaccagatgtcaatatggaggaggcacatgtcagcggcgcctgggaagcctgagttccagctttccctccccacaaccctgctctgtaactgtgggcagagcatgtctgctccaagagcctcagtttacacccctgaaaaatggagttaatgccttacagggattttatgagccacagtaagagattatgtgtgaaagaaaaataaaaagcattttgctattattcttagggtatctggccacatttccattatctctgactaaggtaccaaccaccgatttctgagttcagtaggagacagtgagacagaggccaaggaaccccaaccctgggcctaaaagtggtccagtcagtgtttgcaaactggcggacactcactagtgggtcatgcaattgattccatggatcacaagcagcattttaaaaatgaaataaaatagaatgacaaagaaaacagtggagtgttttgcatggtgtcgggatactttttcagttatatggaattgctagttttatttgcaaatgtagcatacctcgttgtctaccggacagcagagtaaaacatggttcttactgtgagtccctgtcaaataggctgaagccagagccctcttggagaaacagagataacccaggcctgcctcccatccgtggactcagatgctgagggatggctctggcaatctatttttgcagctccctaagtgactgataggtgcgcatgccgcagtgtgtggcaacttccagtgcaggaaacacaggcagtggaacccgaagacctgaatctcagtcccaagacccccacttacctgccccgcatcatcagacaagtttcctaggccctcggagcttctgctgctcacttgtgcctgtgtccaccccatatggtcatcaagaggatttgagctggacacgttaaatgcaggatgcgtgcagccaacagtggcatgctggcttttgaggtaagctgcaaccttctcctttaaagttagcgtggctagtgagaagttacctgagccccatcaccctcccctacacacatgtccacagcagacaccaggctatggggacagccatggttcttgaggggccttggctgacccctagatctgtaatgcttttgttaccggaaaagggtcccgatccagaccccaagagagggttattggacctcacgcgagaaagaattcaggacctgtccatagagtaaagtgaaagcaagtttattaagaaagcaaaggaataaaagaatggctactccatagtcagagcagcggcatggctgctcagctgcttatacttattgttacttcttgattatatgctgaacgaggggtggattattcatgagttttccaggaaagggatggcaattcccagaactaagggttccttacctttttagaccatatagggtaacttcctgacattgccatggcatttgtaaactgacatggcgctagtgggagtgttttttaacatgcgaatgcattataattagtgtataatgagcaacgaggtcactctcgttgccatgttggttttggtgcttttggctggcttctttactgcaagctgttttatcagcaaggtatttatgacctgtatcctgagccgacctcctatgtcatcccgtgactagcaacgtttaacctcctgggaatgcagcccagtaggtctcagcctcattttacccagccccccattcaggtgaagtctctctggtttgaacacctcagacacttcgtttagctaaaaaccattgaaacagtgtattgattccacaaagaggtgacttgtgcttgcatttatccttttgttcccaaatctgttttcaaagttttacatgttgttttgtgttcctttgggtattttttttttttttttttttttttttttgagacggagtctcactccgtcacccaggctggagtgcagtggcacaatctcggctcactgcaacctctgcctcccgggttcatgccattctcctgcctcagcctcccaagtagctgggactacaggcgcccggcaccacacctggctaatttttttttttttttttttttgtatttttagtagagatggggtttcaccgtgttaggcaggatggtctcaatctcctgacctcatgatccgcctgcctcggccttccaaagtgctgggattataggcgtgagccaccgcacccagccttcctttggtatttttttgttattgttttggtttggtttgggttgtgctaagtgtttcaattcagccagttttaactggctgttagatattagactgcaaaatactccagtcagagcagctcacctgcgcagaaaaatgagggttcagcttttagaacctaagttctttcccagagttagaggaactgcctgtgaccttgcccactccagccacctgcctgaaataatgacaagaccagcctgaccccagcttcatgagcaattgtccaaagcccctttgtgccgtagtaattgagcaccagcaggagctacaacgacctggctgtttcttttttgctaacaagcttacgtataataaaaatggctctagcttggggatcttattaatcgcctcatgacaatccctctccttttgggcttctttgggttctgggatttttaaattgttcccaaggagagtttttgggaaacaaactccctggaatcatgcaggcttcccactgggacaggaaagtcaaaggccctttgttattggctggcctttggctgtcagcccttgctatgaaccactggcctgaggctggaacttcctgctgtctgcaccatgggattttgaatggcacaagaatgaggctatatgaacaggaggagctgactgccactggtgggcctgggagcagcgtctccacactgtccctgtgcagagcccagagtgcccagcaaaattcatcactctccagatcaggggctggggacagtggcccacacagaccaaacactggagctacagggcatgtcacccgagggagatgcaaagacctcactggggctactaaggctggctttccaggcctggatgggctcccattgctgcacctcctgggaaagcccacgacacagaggtgggcgccaaaaggctctcagtggggctttgtctaaggaacagataggtccaggtcttcctcaggcaagtgacctgagcagaggggcccgtgtggacttttcaccctgaagctagaagggtgtggtgacgacagtctcctaaatgagtggctttagtcctagctgcccatcagcctcatccgccgaaccttaaaaaacaacagatgccccgccccaaaccaacttaatctgaatttctggaagtggggtttgggcactagtattttacccaagctccccgggtgattctaatgtgtagcaaggtttgaaaaccacagtctaaaagccacagactgttaaactcttggggataaagggcatcacccagccatcttggcaacaccaataagtccacaatgaggaggggtgggagggcccacagttagttagggctgggcacctggctggggaacatcagggtgattgacttcaagacttactcttttttttttttttctttttttttgagacggaatttcactcttgttgcccaggctggagtgcaatggcgtgattttggctcactgcaacatccacctcctgggttcaagcgagtagctgggattacaggcatgcgccaccacacctggctaattttgtatttttagtagagacggggacggggtttatccatgtcggtcaggctggtcttgaactcccaacctcaggtgatctgcccgcctcggcctcccaaagtgctgggattacaggcgtgagccaccgcacccagccaagacttattcttgatggtcagtgctggggacttttttaaccacagtcttccacactaagagattagaatttagaaaagttggggtggctcagaagttgcaggggatttggaattcttgacagacacttctggaagtggcaaggatttgcttatgtgcaactgacagtcaaggagtcttctctttgggaggtgaccacattaaaatgataggaattcactgtgggtgacagcataaagccaatcgtggcttcctcactcttcgtggtgagccagaagttgttaaagactgggccaagaagattccaaaaatggacacaaagccaagaagaaccagagagggtgaggaaggatagagaaggttttatttggttattttacttgggcccaagaatgttgaacgtgagaacctgtctgtgaaattgtagtgtgcagccattggaaaatgtttattattatctttagcaaaagctttacaagcaattctcccacaccatgttctttcactcactgacgttcactgtcctggtgtctgtcctggagcaggaaatgaagggacacacacacacacacaccagggaggagcaagaacggggcccaatgtggaatcagacccacttagaaacacttctgaggggagtgcaaatgagaagcgtctgtcagccgaggctctggattgatgccaacaagctgggctagaagggaatccaggaggctcagtccatgcagaccacccagaggtctcagtgtgccgccacacttaaattacaccctcgcaatgtctccttggagaaaatgcatatgtgttcattgcttgatgggtcatcgctatttccatccctgtgtaatagacgtgggaggtctctcccaaagctctcagtggctcaccagtgctagcagaattcagtcagagcacccctgtgccacgactccctcgctctcggtgccctctgctgtgtcacttgcctgccctgtctaggggactagatggaggctactgcaactactcagcgcttccccctctgactttagctttggaaattacttatctcccactgaagttgattaagtcgcctgcccagggttcatatcagttgaaccagcaccaacagtagaacctgcatctcttccttcgtggtccacgagaactcgtacatgccaagaggctggagtcatagagcagcacttcccagccattttcacattgcatctcttacagaaagtcatgctttgggtggtggatcagccactctcggctggaagtgacccacaaaagcccacacattgttcaaggggtgtgaagctccattgtaaaatggccgggaccatttccatttagagttgggatggtctcttctcccaacggtcactaatctcctccagatggaagagtaggcccagcggggtttccacccgggttgtttataacccagtgtggggtgggaggtgaaggtctagcatgcacagagggcatctgtgtggtgttctggggctttctatacggtgccaggtcacccagaccacaggccccgtgagtccccttcgctactggtgccaaatccacgtgccatctgtaaattatttactctgcatttatcttcaagctaatttaaaatcaatccactttttgtctgatcttaacttaggccaaatgttatctgtgaaattatgattttggtatgcaagcagacacttgttactctaatatacattaaaatacacacataactagtaaaatttatacacacacacacacacacacatatacatgtatatgtatatatatatttagagacagggtcttgctctgttacccaggctggagtgcagtagtgcagtggcacaatcttggctcactgcaaccttgacctcttgggctcaagtgatcctcccatctcagcctcccaagtagctgggacaacaagcacatgctgccacagcctggctaatttttttgtattttttgtagaaacagggtctcactatgttgcccagggtggtcttgaacccctgggctcaagcagtccacccacttaggcctcccaaagtgttgggattacaggcgagagccaccgcagcaggccttaaatgttttttaaacttctttgaacttaattccatctgaaatcatccagcagcctccagagataacaaataacaaagttaaggaaaaagcatggtcatatagttattgtatggaatccgtgtgaattggtgggtccttagacaccttcagcatagaagcattattcccattttcctgttgtggaagctgaggcacagagagttgagagttatcataagatcacactaagagagtgacaggactggggttccaacccatagcggtgagtactaaagccaccctccttcccctccgccaggccagcagatgagtcgttggcaggatcactgcatctgtcatcccctcgccctcactccagcctttttttccctccacagtcctcactgggctttgtggccctcgtgctgagcacactgcacacgctcacctacggctggacccgcgccttcgaggagagccgctacaagttctacctgcctcccaccttcacgctcacgctgctggtgccctgcgtcgtcatcctggccaaagccctgtttctcctgccctgcatcagccgcagactcgccaggatccggagaggctgggagagggagagcaccatcaagttcacgctgcccacagaccacgccctggccgagaagacgagccacgtatgaggtgcctgccctgggctctggaccccgggcacacgagggacggtgccctgagcccgttaggttttcttttcttggtggtgcaaagtggtataactgtgtgcaaataggaggtttgaggtccaaattcctgggactcaaatgtatgcagtactattcagaatgatatacacacatatgtgtatatgtatttacatatattccacatatataacaggatttgcaattatacatagctagctaaaaagttgggtctctgagatttcaacttgtagatttaaaaacaagtgccgtacgttaagagaagagcagatcatgctattgtgacatttgcagagatatacacacactttttgtacagaagaggcttgtgctgtggtgggttcgatttatccctgcccaccccacccccacaacttcccttttgctacttccccaaggctcttgcagagctagggctctgaaggggagggaaggcaacggctctgcccagagccatccctggagcatgtgagcagcggctggtctcttccctccacctggggcagcagcaggaggcctggggaggaggaaaatcaggcagtcggcctggagtctgtgcctggtcctttgcccggtggtgggaggatggagggattgggctgaagctgctccacctcatccttgctgagtgggggagacattttccctgaaagtcagaagtcaccatagagcctgcaaatggatcctcctgtgagagtgacgtcacctcctttccagagccattagtgagcctggcttgggaacaagtgtaatttccttccctcctttaacctggcgatgagcgtcctttaaaccactgtgccttctcaccctttccatcttcagtttgaatgactcccaggaaggcctagagcagaccctttagaaatcagcccaagggggagagcaagagaaaacactctagggagtaaagctccccgggcgtcagagttgagccctgcctgggctgaaggactgtcttcacgaagtcagtcctgaggaaaaatattggggactccaaatgtcctctggcagaggacccagaaaaccacactggctccaacttcctcctcatggggcattacacttcaaaacagtggggagcaacttttccaccaaagctacaaacctaaaatgctgctgccccaaagcacaagagggaagagcaccgccggggccacaggacgtctgtcctccagtcacaggccatccttgctgctccctactgactctagcttacttcccctgtgaagaaacaggtgttctcggctgagcccccaaccct

SEQ ID NO: 5 - DNA, 82154 bp, Homo sapiens
cattaatagtgataactattaataatatctgagtgttaactatgtatcaaacactgtgctatgggctttctttaactatctcacttatctctcacaataaccctgtgaggcaggtactattacagaaggggaaacggaggctaataaagattaaatgagtttcttagggttgcccaccaagtggaagatcctgaatatgaatcttacagtctttctcagcctgcattctttaccacaaggctgggactcccagaggacagcatcctctcatgcctgggtggcaagcagccagccacagacatgctgaagaactaggggtagctacagttagaaaacattcattgctttttcaaggaagagctaaggagacccaggacgcagatgtgagttagcagagaacaccagcaaattacttaacttgacagaattattttctctcctgggtcagttagcttctacttgaccctggtgcttgttggagtttgtgtcacccccatggctctgccttctgctctgaagcacaaggttaccattagcatacatcaggctgtggttaatttggctgccccctctttaagctaaggagacatggtcagggcctcagaccctgtgtgtggccattcagctttgccctgttctgtggccacagtccatgctctaagtactgaccagatgtcttacaatgcacacggcacacagttatggtctctgtggggatgggggaataaggtgtggacaaatctgttcagctctaaatccttgaccagaggagcagctcagagcatgaatccacagggcagtggagccatgtgcaggatcaagcacagcggcatcactgacttgcaggacactggagtgctggggcgtggttctagagaaatgccctcaagtcctcgatagatcctaaatgtggttagttctcttcattacaaagacagtggaagctgaggtccatgcagattaaaaggctaagctgtacagtaattatgaggtccttttgattagcagaactccatctattccaaaacaaattcatagaaagacatcttttctatgcctaattgcctttccatctttgttgaaaatcaacagaccataaacgtggagtctgttttgggacttgacattctgttctgctaatctatgtgcctctctttgcgccaatactttactctcttgattactgtagctttttagtaaatcttgacatcagatattttaagtcctccaactttatttttgcttctaagttgttttgtgtttacatacaaaatttagaatcaatttgttcaatttctaccaaaaaagtctgcttgggttttggttgggactgcattgaatccatagatcaactattggggattaatttttttctctacgttttgactggtttgtataccacagattagaggattctgggatggagcttcagaatccatagcacaaacatgaagatattcagaacaattaacttttatgctgatttaaagctaataagaataaatctttacatcaaatataagccacctggagccagtattattgggacttcattgataataagattatttacccaaaggggaatgattgtcaaagtacctactgtgagccatatttataaaatgtaagccatatttgaaaaatacttaattttcacaagaagctgatgaggtaggcattacctgctgtactttacaaatggggaaactaaagtacaaagagatgtagcagttaagctgaggtcacacaggtaattaaatggtagcattgtcacttaaacccaggcttgtctaattccattctgtggtacttgctgtacctcaatggttttcagactgtgtccagagtgccccaggacctgagagagagagagagaaagaaagagaaaaaagagagagagaaagagaagagagtgagagagagtgagtgtgtttgagggaaggaggaagaaagtggtctggggttcagaatcccagacttggtctcagtgagttttcctttgtcctattttgcagatggggcctccacgtgaaaagtaactacaaaacagaaccagaaaatagtcatgctgttcaagtgagttggcatatcacttcatgctactttgaaataaaatttcctcttattttcttatggataaaggtctcgaaaggtttgagatgatcacagcctgtgttaaggctgaccagggtccagggcagtacagtggagctgcttttcttctggattctgaaccaccctcagccttcagggcctgttgtctggtgggctcatggagtcaccagtgaacgtgaaagccactgctcctctggtgtccatgtctattctgacaggtggatctcagccccaggttgactgattgctcgcaggtgaggctactgcctccgcagtgctcaccaaatgctccggccgctccagtctgcacgttcctgagcttgggccacctcctgcttttcacaggcagcttctcctcgatctcctcctctcttttttcttcatgagcctcacccttggcagccaccccaagaaattcagccttcttcatctcctttctctctcagaaactggaaagatttttactgaattcctgatatttttgtctttgccaaaacccaaaggccccaatctgccatgggccaagaagtgcccagagaggttgtttggacaaatccagaaatgaccttgaatctttactcagcacttagatgacaggttctctccttctccaccacaccctagcacacgtgcttcttgtcggatcaggaaatgttttattgctttttttccttttcactttctctacccacccttctttgtgagcccttcccggttatgctgtccgtattgcctctcttctggaaaagtaatcagagagtgaaaatcccaggacaggaagggaccttgggaaggcattaaccagaccttttcctccaggcagcagccacagtcgccggggtaaagaaccctcttttccatctttaaactttcatcggtttctgcatcttctcattgaagcatctctgggttttgctttttgttgtcttatttgttactttcatttttaaaagttttctttcattcagaggtttcttgagactgcctggtgcacatcatgcgtatccgctgatagatgctgttgtgacttggtgaaacatgttgcgtgctcttcaggcagatgtatgtcaacaaacccttttcctggctcagccaggatttctgggagtcagatttgattatggggtgtcataggtaaaaactggaggctgtctgtggtggcccagctgtgcagttagctgacttggaggaatctttggctataattggatgccaagagtggaggtggcacgagagatggtacaagcctatgatgatgtctacccaggtgaaaaatgatctaggtgcttgtaaccatcaccctgagcattcagttctacatccagctattgtggaacaaaatgatttatattgattaattgactagttggttcattttagcttgaagagtactgatcagctgtgtgcattaggcagtttcttgacacactgctgtgctgaggacagatttcccagagcctggatgtgtataattctacatgctcctttttccaaggacttaagcaaacagagtcaactcctctgttttccttgtttgggtatatttttgaaaatgtctcttcttctttcctttcatttacattttaccgttgtttatccaccttttcctcttccttcaaagctctagacctgcctccctgcctgttcttagcccacttttgagaaaggactttccaaccccatcctcgaaagggcagggtctctctccagggctccaacagagttctgaaagaacacggcttccaccattgtgtcttctctgttttgtcaactgctgaaagactgacacagtagacaatgctgcagaccccttgggagatagtccaagtttagggtggggagggaatggtgcattctgggtgtccacatatttgttgttgctttaggaagaggagctgggaggttggagtcgccacacatgttgaaagatgtggcaaaaaacaaacaaacaaaacacctcatagagagaaatcctgactttgtgcacaagaaaagggagtgaagtgtgtggggtttttaaagtggtggtgggggggcattaggagatacccagaagctctcaagattccaaacagtctgcttttcttttcagacattacactccaagaaaagaaactctagtgtataacagaggggctctgtcaaaatttaacaacataaatattgggcaaatgaagttgtttctctggtgctatctcaacctgccaatggagaaatatctcaaaaagtcaagaattgaagccattactcagagacagcatcctatggcatggtgatccaaaacaagtacacctctaccttttccctttcagccccaaaccccaaaacactggagaatattggggcaggcaaggcccgtgaataggtgagcagagctggaagaagagactcctgcccctggtgggcctcaggctatttgtggaatgatgtttgtgagatgaacatgttcgctccctcacccggatgttttggacataggagcctggagtaaagagtgtatatcttgggctgtcattttcccgtgtgaaaggtgttttgttttctccccgtttgcacaacttgtttcccttaaactgtgctgtgcatggctaggaagctcagcaatagactaaacagtttgcgatcccacagttttgctttctgtttctcaggcctgacccaacactccatgggttccctagcccttcgtaggtgcttcgaaacactcatggggtccttgacctgatcccagtgggggtttgtgtgtggaagttagggaacctctctttgagagttctggtgcccctaactctgtagggtctgacttcacttctgccccaataagaatgaaaggctttgccagtagtgcagccctctcctcccaaagtcctagaggcaattactctgaaatatggaagaaagaggcacatctgaaagcccagtgctgtcttcttgtcctcacatccctcaggatctcaaggtcagcctagtagaggcattgctttaccatagtagcaactcagggaatatctgtggaactggattcctctgaagttggacagttgctttatccctgctagaggctctcactacttagacccacacagtcctatagatagaagaaacctagagagaaccacctctcacacttttattttatacatggagaaccaaaggcctcaaatggtggagtgatttgctggggtgctacagtcattgtcacaacttacattgtgacatgggcttcatatccagttaaaacaaaagcctctattcactgccccaaggtgtgaagccttgtctccaggagatggagagtcaatgactccaaggagataagacttcagactcttcctgctgcaatttttttttttttttaagtaagtgccaaccaataactcctggggaaagtaaaataatctgggggaggaaaattgttctctcctggagatgggactcctctgatttggaactcctctgtctgctttcacttcttggataggattttaaatccagcacccagcctgctgctgtgaagttcacaataactttatcacctgagttcccttggtgctgcagcagatatgagttacacacctggtcctccgagagtggtttaatccacaccagagtcctgacttactagtttcttcctattgcagagcctatgcagactgcttctctgagcaccaacatcattatgcactgtcctttctattcccaagacttcctagttgctgcaagtcaagttcgacattcaactatttcctcggggtccttttttgggagaagcaaggctttgagttcactctttgacttcaagtgaagtccattttcctcttagacactaccccagtggttctcacactcaaggaagcaccaaagtccctggaggacccgtctaaatgaaggatgctaagccccacctcacagagattgatgcagtacgtcccaggaatctgcatgttaaaggagctctgcaggtgattctgatggaggtggcccttgcaggatgctttgagaaactatatgttctatccctcgaactctgcctgcagaacagcctgagatggctcttccatgctttgaccatttggccctccacagtagtccctgagctgcgtcttcttcggaccatgagcagaatgtgtgtcccctgactgactgtccttttccttgagaaccaggcatagtggtgcactggtaagagcatggactctggagccacactgggtttgaattatgtctctgccacttactagctgagtgaccctaggcaactttctaaatctctgtgcttttgtttcttcatctgtaaaatggggataataatactacttgccccatagggttgttatgaagatgaaccaagttaatatctctaataaatgtctctaaaatcccatgattgcaaaaagtagaaaatatgtcatgtagcctattatatcattatattataaggtgaggctattgaaggcaagaggggaaatgacttgctgaaggcaacagagctggtctgcagcatggccaagaccaaaaacagctcttttcgctcccagataaattgtcttttttatttttttcccaaactgcctgtctaacccccctttcatctggttataatgaatgccattgcttgtgtatagttgcctacattaatagacaacaatgcaatattctccttatggaaaccctacctagattgggaggcaccatgaagccactttttcttctttctccatctctaccctgattagcaaacatctctaattattgtgactcccaaccccagtcagatcattttattctttttccagtatctatgcagggaagcctggcaaaggcttaagggatggggtggggtgtagcatgtcagcaggatttggaactatgggccccatacttttggcacctgctgcattttctccccacctgctgcaccataattcatctgcttgtatgagtttagcagaaagagcccccagttccctgtaagaaacacatacggcttttccaggttacactgtggggattgagaaaatgcacagtatccatgtctggaaatcaatggacaactcactgcctgaatcgtttgtgtcttgtaacttgcctcaagcaacatctgtctgaaaaatctggtgctttgggctaaatgactggatattctcctaaggaatggcagtgagtgggttaatttcacctctcatcttacttcctgcctggcccctctaccttgcccaccatgcctggctttgctgctgatcttcggctgacctgaaattgaacaccttaacttaaggacaactaagggtgtaggtagattgggaagtatgtgttcttcttgctttgccaataattgtcttctggattcattggcctttcttctggtttccttcttcaacttctttaacaaatgacttcagccatgccaagccttcttttaagcactttccctctcactctcctgtcctataaagatatctttcttgaagcccctccgtgccaggcgtccaaagtgaggcacatcaactcggtccttgaacgtctcatgtttatcccaatagatggtctagatgtggatgaatgtaaaggaaatgtgcaaataagatacttttaaaattcagacagaattatacagggtgggaaatttcagtatacatagaaggcttaattcaaaatgatataatgtcaacatttggtataagaaattatttatctggcagaatatatactcacagatgtcaaaatgggtctagagagtagagaatggaggcaattatccagacactgaggcccaaggcacaatacttttaagtaatcatgccctcaactggcatttccatagtcttaagcccaaaagcattgggtggaaccattctataagcaaagcctcagctatgaaaacaacttttaaaattgacattcaaaagataagatcaacaacaggtgccctttgctttcggcagtaggtgtgttcttgaaaagttggttgtcaatatttccttttcccattaatctaatcacattctaaatataacagggggctggatgtttgaagaaatcctgtagttgatctcattgcaaagtccttttagagaggtaaaaatagtttgttttattttcaaaaattaggttttcttagactaaaacttcttggaagtgggggcatatcatgttcctgtatattgcctacaggtaaagaatgtaagaatgtatggcttagtcacagagagctagggcttcagaaatgctatatgtatcaaatattttatatctatatttactctgaagatatgacaatagcttttaatgtgggtcgagagatatctaatctttcgcttctctttcaaccttccaaaaccatgcaatcctcattccttcttttcaataacctccacatgcccccttcatctgtcccacccaaaggaaactcttttaacttaccatttaattgatcaactctgatgacctttccctaagctctctgtgctaaaataatctggtactttgggctgaagaagtattctcctaacgatggaggtaggtggcagtcaaatgacgcagagattatgatagggccctgtctaggctgtcacataaggtaacattagccacatgtggctactgtgtacttgaaatgtgatgaatgccattgagaactgaatttttaatttaatttaaaaatggagtcgcatttctgttttaggagaacttctaagtatgtctgaaacaacttgatcatgtttcaagtgtaagttttattagatctaaatctaaatcaagtatttcgaaaaagtattttagcatttgaatagagatgtgctctaagtataaaacacacagtggattttgaagattagtaagaaaaaaatgaatgtaaactatttcattaataaatttttatattggttacatgctgaaatgataatattttgcatatattgggtgaaattatatatattatttaaattaactttcctttttttttttaaactttgtgtggctacaaggacactgaaattagaaacatggctcgaattatgcttgcactgaacaaggctagtctaaggtatgcaattgccctgcttcttcactactttcaagtgtaaacaaatatgtcagcattatccctagaaggagggtttaggggttttaagttgaatactcttcagctgaagaaaatccattaatctccagttttattcttgagttagctttcaaatctacattgcgacaataattactttgggatctttgaattttctttcatgcctttctgttctatgaagcccacatgcgtagtttatgcattttccaatttaggctagaagtccaatgtgctgtccattgcaccacagagcctgtttgctatgcatttttaagagtaaaattttcagaaaatacttaaattaaaaagatagatttggaaagcatatccaaaatgttgctttgaacttcttattctggacaattcctggactggggagaaggcatgcccaagtacacattgcttgcagtggcacctccaaaccaatgctggtcgggactcttcctattacctcgcaacagcaatgactgggccccataatacagccacggtcattataaaagatcattacaaagcatcaaattaactgctattaatgaaagccagaaacacgtcattcaggaggtcgaatggctgggatggaacatacagggttgaataatggtaggagcctgggtttggaattagaaaactgggacaggaatcctacttctgctatgaatccattgcaagtgactgggcaagttgttcctcactccctactgccttgctcatctatcagctgagaatgtcaaggcagaaggtctccagggctcctgcaggctcctacaagccagatctcaatccgtcttatcctaccctcttcctgacctctagctacaagaacagcaaggcacagccccctggttttgagaatgctaatgcttgccagcattcttgtcctgcagggcatgaagtatagtcctgcacaaccagcaggctctgtgactgcctaacctcatccttggtcactgacctctgtcttagtagcagttcgtgccatcctacctagctttgctgggtcaagtgcgttcacatcttaaaaccgtttgaagcgtttgtctaattaacatgcagaaagcatatactgatttttattttcaccaccatgtatgtgagggcctattttatggcatgcaaagcatctcatttttatttcaattctctgtcttgtaactttaaatgaggctgaaatttccctccatatgtttattagcatcttctattttatctcttatacactgactgtttgcatcttttgccccatttccctgaatggtttttttctcaatgatttgtaagcactctttatatttaaatatgttaactctttcttgtcacatattttaaaccctttcttacaaacgttgttttccggtttgtgacttaccttttaattttgctcataaattacttttaacttacagatagtaatatttttgaaaggttacattcatcagtctttgtattgttaaaaagagtctttattgttatacttttaaagtagcttttccactcctagtcagatactttcacatataatagttgtctactttcctcctgaattaatggggatttattttggcatatggtgtgaagtaagaagctaaattctttttcttttctctgcatcatcaatgatctcttacactgttaaataaactagtcttttccttctgctttccacacccatttaacctagcccactgttgacatcgtcagagaaaacttttcatcagaaagtctgaacagcacaattatactgtatcaatccagcttaaattacacatgattgttaggatgggtagaaggtatgataaaatggggtgaaagtaaaatgaatgatcatcgatatacatgatgtcaaaaatttgggtataactgaagaattttagtaatttaaagatttcagaatttaaataatgaaatatctgatacattcaattggaaatttggttgagatcagctgacttgttaattggacttcactgataaacatttgccttaaaatctgtagaatctaaagcagggtatacctagtatgcgagtaagacatcatttctggaatcaagggggcttttcagtgttgaccagatgtttatcactgcatgggcaaatggcccaaggagtatggttgtggcttggcttatttcttcttaacgcagatgcagttgctttctttctgggtggcagtgtggaggataactggggtcggtggggacataaagtctcacaaggggaggaaagcccctctgtgggctctctgggccagccaccctccttacaaagtgggatgtcagcttctgctctgaccagagtcaaagtaggcatcagctattgccattactgttgttgtataatatggtgaataaaggaaatgtgttatatatatatttttttcttattattatactttaagttttagggtacatgtgcacaatgtgcaggttagttacatatgtatacatgtgccatgctggtgtgctgcacccactaactggtcatttagcattaggtatatctcctaaagctatccctccccccctccccccaccccacaacagtccccagagtgtgatgttccccttcctgtgtccatgtgttctcattgttcaattcccacctatgagtgagaatatgtggtgtttggttttttgtccttgcgatagtttactgagaatgatgatttccaatttcatccatgtccctacaaaggacatgaactcatcattttttatggctgcatagcattccatggtgtatatgtgccacattttcttaatccagtctatcattgtgggacatttgggttggttccaagtctttgctattgtgaatagtgccgcaataaacatacatgtgcatgtgtctttatagcagcatgatttatagtcctttgggtatatacccagtaatgggatggctgggtcaaatggtatttctagttctagatccctgaggaatggccacactgacttccacaatggttgaactagtttacagtcccaccaacagtgtcaaagtgttcctatttctccacatcctctccagcacctgttgtttcctgactttttaatgattgccattctaactggtgtgagatggtatctcattgtggttttgatttgcattcctctgatggccagtgatagtgagcatttttttcatgtgttttttggctgcataaatgtcttcttttgagaagtgtctgttcatgtccttcgcccactttttgatggggttgtttgtttttttcttgtaaatttgtttgagttcatggtagattctggatattagccctttgtcagatgagtaggttgcgaaaattttctcccattttgtaggtttcctgttcactctgatggtagtttcttttgctgtgcagaagctctttagttaatgtgttatattttaagagctcagttatctgttacctatttgccagtgacaggaactcatgtcttctgaggcagttggtctcatttatggtagatataattaattattatctaattattattgcccttgaattatttgaagatatcatcaggccctctcttaaacacttccgttcatgtgctttagcttctttatatggccagccactctctgtggatatgtccaggtttgttgatgtccccttgacatcagccaaccataacttaacactggatgagagctaagcagtgctaaacttatcacttccctcgaatgtgtcattatacttttattagtgctgcttgggactagatttcctttctagcagccaagtcatgttttgggctcatatgcagcttgtagctaagaaagccttgagttgttttcctataaaccgtctgaaactatttttagaattcaagttacaactctgaagtcctgggtcttacttaatcaatgataccttctgagataaatactgaaaatccttcaagtatagaacaaggacatcctaactctgggcatctgatgtctgttaaatgtatcacattttatttatttgtattttttaaaggaagtgagaaggagaagtgagatgctgagcatcaagctgggcagagttgtcatgttgtgatctgagattctgtcctgttcttcaggatttaaggagtcgctaaccaatgccaagcatgggctcatcgggtgcagctggggaggcaattacttcaactcaggagttttcaaaaactttccttatgcctgctcacaggatttcatggtgtttttcttctttgttgttacttttgtggaaaaggaaagttaccagtgaacaatctctgtgaccttcctctcaatcccaaaaatatgcttgtgataaatcagcagtttattttaatgcctcatttggaggcattgcttttccaagaatgaatctttgcatattatctgagacatcattttggtgtaaggtagaattgtaccaggtatgtatctttgtggccaactatactgtcaaaactgagtttcagtcatccaataaaaaggaagctgtctctgaattcagatattcctccattctaagggaactgaagtataacataaaatacttaacattctttctgtatatgtgaaaaaaccatacctaaggaattttattattcagcccatgtttctttattgtatccagtataacatactcgaagatgtatatatacacatatatagatatacaaagcatctatacatctagacatgtatatatacatatatatatacacacacgtatatactgtacttcaatcatctatattaaattgtgataatatcacattaccacatgttgatggcattcttgtaatctgcctgaaatccatgaaaacacatgcagtggtctggctcatgttgtctcagggagtgcattctggctcccagtgatcatcaattacttttataaaagttcaccaatcacttgttggtttccggaaaacaaatttaaagcaaacattattaatgtcaaacttacacattttgctttctggaatcaacttttttattttaaaaattgagaatataatttctgtcttttgtttctttgggcatttttcttattctccacaattattcaaagatccttagccataggtctgaaagtgtttgtttcagctctcatcccccttccttgcaggtaaggctggcattccacagccctggataactcctctgtgctaggtctatgcctgctcctgggacactgaacactcctaggtttgcagggtccacggtgtccttcgtgtccctccctgcccagcctcagcaggagccccctgctcctagaccactcccttactgagtcctgtagatgttccagcacacacagtgcctcccttctcaaggccccagccccagccccttctcaggtcatttgggggcttacatcacctgctacctgtgtggagatggaggtctgtttgccagcactgccttgcttttcctcctgccccactgcatttaatcactgtgataagaggaaacaaagagggaacgtcattggtcccattcctctgaatcttgaccacgaaaatatcttgaattgcctccgtgtccccacccaacatcgcctcttgcactgctacctctgcctgagtgcacatcagccaagagccaggaaccactgcccaaccacgacccaggcacagacccttatctctagcctagagaagaggtgtgtttctttctaatcatctggtatgtatccaaggaaaccataaattgatcattatttttgtatgtggaattagaaatattcatatagacatacattctttggtttttatttttttatcatccctaaaataaatagcagatgttctcaaaatggtttattaatttcagtcctaagattgtataattgattgaaaccataaatggcagacagaagaacagcaacagtgaaaaccaagattaattctaataaattcattacaatggcaggaatcaagtgtactttaagctcactgtaaagttttcagtgtgttgtggattcagaggcagtaggtgatgacttttattatatattcttaagtacctggttttggagggtgatggcttgttggagggaatgatgctgaaggaacataataaaacaatatgataaatacagggagagaagaaagacaaaggaaaagaggaaacaaatgtcaagaataagatgaataggtaggggagaagaaaaaataagttgtcataattttatattccaaggactacgctagtccttggactacgctggtaaaaagaaatctaatcatgaggcagagaattttatttatttccccattccttttggctcgtggcttgagtttgctctggctttcattgaatacatgggccaaggcaggatgagcttctgcctgaaccgctgggatgttattcctgatctcatggaaagacatgtcgcggcttctgaccctgtgcctctgtatcactgtgttccctgtattcctgtgcccctcgtattagttttcacattgctaatgaagacataccagagactgggtaatttataaaggaaagaggtttaatggactcacagttccacatggctggtgaggccttacaatcatggcagaaggcgaaggaagagcaaagggacttcttacatggtggcaggcaagaaagagcatgtacaggggaattcccttttataaaaccatcagatctcatgagacttattcattaccaggagaacagtatgggggaaaccgctccccatgattcagttatctccacttggccctacccttgacacatggggattattacaaaatcaaggtgagatttgggtggggacacagtgagactatatcacccctgtatccctgtgtctctatattcctgtatccctggctgaatgggtcaaagtaatgatcagcccacagatatgcatttgcctctgctgagaatagaaaggaatataaatgaagtctttttcttcatggagcttattaactagatgaagagagaaagcctgtgggaaaagacaaatagaaccttcaggttctatgcagtgagtgcccaagtggtgattgtgaaaatgaaagtgcagccctgcagatgagcagaagctcatggtgggacctttcatgcaagctatgcagaggagagagcataggatttggggttgggacacacagattcaaatcatggtcctgccactttcctgctacatgaacttgaccatgttattttacccccatgaagtctgagttttcagatgtgtaaaataataccactaatgcatacttttttaaaatttaaatttaaatttttttttgacactgagtctcactctgtcaccaggctggagtacagtggcacgatcttggctcactgcagcctccacctcctgggttcaagcaattctcctgcctcagcctcctgagtagctggaactacaggtgtgtaccaccatgtccagccaatttttgtatttttagtagagacggggtttcaccatgttggccaggatggtctcgatctcttgacctcgtgatccgcctgcctcggcctcccaaagtgctgggattacagatgtgagccactgcgcccagcctaatgtgtacttttaaaagcacttttcaaatacttcatcttgtttaatcttcttggcagttctgtaaagtgtgaagcattgtacaaagttaactctcattattatattggaacccagggttttcagaggagaatcctcaaggagatagggctttcctgccttgtgcccatataaggttgtggatcccaagggacgaaagcattttcatcagactgggagaggcaagaaaaggtcactggcagtatagattcttctggagttcactggcaacttgtcccacttcagttctcccttaatctttccagatgtgcagatgtatagcctgggtggatgagccaaaatgaattcaaggctaaacaggtcagctggctcagcccatgggaagcatgctcatcccaaagcaaaggtctctggcagaaatttctctgcacttatttaccctgctgtcttcacattggggtgcggagtctaattcaaaaactccaaggcatgagtccaagccagggagctgttagaggtgcacatgggagatcttagcaagagaaaaatatatgttttaaaccctagaaagaagaaagtaaatgcctctcatttactttaaacaaataatatggccaaggtcacatagcaggaaagtggcaaggggtctgtcctcttttcactgttcaatcatgaccatattgtcctggaggcctaggggcaggatataatgaagaaacatccaattttgacagagtgggtaaatgggattgtgggacttgtagtgagctgatctgtcttcattcatgtaattatcaatatcatttcaatgaccatgatgttgatatggaaaatgatgaggatgatagtacagaaagaacatgaatattagctgatgtctattgagtgtttcctgggtgccagacaccaccgttaagtgctgaacaaggattatagaatttaattctctagtgaccttttgagataggttatatgattgtcagctttccacaaatgaggaagccaatattcagaaggatacggtgctgaagcttatgcagtaagaaatagtagaagcagaattcaaactcaggcaattgaacaccaaaattcaagctcttaattactatttctagaaccctctgactgactgattcctccctcaccacagccaacttttgcatcccccatttctggagagcatctccccacatggtatatactcaaaggtcccatagtgatcctcttcccaaagagcgtgaatgaggggtttgaaactgccagaactctggtccagagcttggacttttgatgcgaatgatggagaaaccaagaggttgatttcctcagccttcaagagagctcatcctcagctctctaaagctgaccagaaagaggagagtgggacctatgaaaacagcaggagatgggagggcttgaattcctaatgctatacccataggtcattgtgccatggtcctctgcacccccaacacccccaacactgatcctgaaggtctcaacaatcttcatctgcctctctcctttcaccttctttacactctcattgatagccttggttttttctatacctccagtattcccattcccatgatagccatttaaggacagggcacttcctggtcaccataaggaatattgtgggaatgttctgctcatggatattgcatcagaataggtctggaaatgaaacacaccaacaaagtcaacagaggagagtccaagcaaagcgtctgtttacaaagttgtggaaggtttaggggaactcattatgagatagttgaagcaacccagagctaggaacagtggcgctcttaccacaccaaggtttgaaggagctcgggtttctggaacccagacatagcagcagcagttgtgatagaggagttcctggcagaagccatagctttcagttgagaaggtggtcactgccaacccatggctcagctggaaggtaaccagagaataaattccccaaactctttctcttcctttcctgtgctctcttattggtgcctcccattgagagaacccaaccaaagccagctgtaagggaaactaatgaggcatttccatatagctcagcctgctgggaaacatgtactatagaaaagggtggagagcggatctagagaggcaaaaaaattaaacaacggcaccgatgtttacatttctttctgatgtagagacctcataccacttttctgtacatcttactttaatgtgtaaaaaggcctataggctctttctctaggttcagtaaaagatgaactaggaaacatatgccacatttgagcttcctcacagcttggcccctgcaaagctgtccccttctaagaactaagtgccctgggtattcagtgctgtgttgtctggtgacttcgcagccctgtcaacattgtttttctgtttgtttgtttgtttaattatactttaagttctagggcacatgtgcacaacgtgcaggtttgttacatatgtatacatgtgccatgttggtgtgccgcacccgttaactcgtcacttacattaggtatgtctcctaatggtatccctcccccatccgcctaccccacgacaggccccagtgtgtgatgttccccaccctgtgtccaagtgttctcattgttcaattcccacctatgagtgagaacatgcggtgtttggttttctgtctttgcgatagtttgctcagaatgatggtttccagcttcatccatgtcactacaaaggacatgaactcatccattggctcctcctcagaaaggtggaaaagcagaacctcttaaactgctccaggtttcctggagagccttgccttaaaaaaggccctttttttggggtgggggggacaccaatgccgtgactccttcaagaatgtgattctagcaagagactgagctctattctttttcttcctccaccctctcttaaggcaaattgagtcaaaccatctcagtctacccacaccatccttctttaatttctgtagttaagagcataaatcccttccctattctgagtgtggttcatcgacctacagcatcagcatcacctgggagtctgtcagaaatgcaaaatcttaggctccagcccacatccctgagccaaatctgccttttgcaagatccccaggtgatatacatgtacattaattttaggaggattgatgtagcctgtatgccatggtaagaggcatgcaatgtaatgttgagtttctttctttagaatttatagtagcagccgggaccggtggctcacgcctgtaatcccagcattctaggaggctgaggctggtgcatcacttgaggtcaggagttggagatcagcctggcctggccaacatggcaaaaccctgcctttaccaaaaaatacaaaaattagccaggtgtggtggtatgcccccgtagtcccagctactcaggaggctgagatgggagagtagcttgaacctgggaggcggaggttgcagtgagccaagatcacaccactgcactccagcctgggtgacagagtgacatagtgagtccctgtctaaaaaaaaaaaaaaaaaaaaaaaaaaaaaagaatttatagtagccacaaaagcctttttgagtaattgaagaaattctgaagttattgtgtgctttgaaatttagtatttattgaatagtttatatctttcaaaaagcttttataactcattggagtctcgtcataactccatgaaatacgtaaggtagataataggagccacattttccacatgaggatctgaggcatgattaagtaaaatcagtcctctgctaggtggaatggttttgagaccagatactagggctcagaactcattttaaagctgttgtggcagctatcaattctgaagaaagtcaacatggcatagcatgcctcttaccatggcatacaggctgtatcaatcctcctaaaactaatgtacatgcacaccacctgagggtcttgcaaaaggcagatttggctcagggatgtgggctggaacctaagattttgcatttctaatagactcccaggtgatgctggtgctgtagctccctccgtcccccagctttcccccatcagtatttctgaacacttgatccacacccaatctgcctcaaaatcacttgtggtggaaaatgcttgttaaacaggtagattgttgtgccccataccaagacataatggatttcttatggtctctaaatgtgagaatcacttttttgtattggtttgaatgttctgatttaagagtatgggatgagggaattttaggttcattgtgttcacatcagtaggacttaaacctatggaagactttgcagcagaataggaagattattctcccagcctacccaacagttgaaaaacgatcagaaggacttggaagggagcaatggaggattgatgacaatggcaaagactgagcagtccccaggcgtgacaacaaaaaggaaagcaatcagctgtggagagagaaatggtgttgatgatattatgaaagtcgggatataaacaagaatgagaaagggaggaatggggaaactgaatgagacagaagcacagaaaaaagaagaaaggaaaaaagaaggaaaaaaggaggaaaagtgggagaagagacagatggagagtgagagaagggaggcaggtaggtaggcagaagaatgaggaagaaagcaaaggagttgagactgggaatggacagaaggaaacctgtacatgctgtgatctgggctttctatgttccctgatccagggtcctgagatcttggggtactcgccactgcattgtaagtttctgtggatgaagctggcatataaatacatctgtaaagctgtaactcctgggcattgtccctgaaattttactgatgtctagattctaggtatgggattaaatgggtccacattttgtgtttttctgagtcaccaattacaacaaaacttgctcatatacttatgcctatcttaaaaaataatttattttaataaaataataataaggcaagaaaaaaagacacccagatttgaaaaagcaaaaaatgtctttatttgcagacaaaatcatgtatgtagaaaatcctaaggaatctacaaattagctactagaactaataagaaaatttaatgatgtctcaggatacagggtcaatatttaaaaattattgtatttcaacttataagtgatgaatgtttggataatggggtttaaataatccatttacaacaacatccaaaatataaaataactagcaacagactttataaaaacatacaatgaaaattgaagcacgttgctgggaaaaagaaaaaagaaaacaaaattcataaaaagacattatattcactgaacagaacacttagtatttttaagacgtcagttcttatcaaatttatgtgtagactcaattcaatcccaatcaacattccaacatcaacatttggctagagagcaaagttggggtcagattgtgtagggctgtgttaaagagttgagcattacatgagataatggatgtgaagaaagtgcttaatagggggtcgtgcagaggaagtgctcaagaaatagtatcattgtcattattaccattactgtattgttgttattatttaacaaatgttttcaagcccactttggcttgtgtatgggttgaggaaaacgctacccaggcagaaagataaaacttattctccatgcctcttgagaaaagaagcagcaatccccagatgtagggaggaggggagccctgctggcttttagtcagcctcacacaggaaaggagaggtagagattctgcagcgaacaagaatcatgtccgggacttcacagacagggacactcaaaggttggatgatgctcaattgcagcatccacctgcaggggcctcaccccacttctcacttaatgattgtctctgctgttagggagcccttgaaagccaggtgttttgtctccttcccaaagcctcagaagatagacgggggccatgagggaggtgggggcagggaagggggagttcctgtctgttcttttctgcccttccctgtgctctccacctctatcccctcttccttcccaaacaggtggcaggaatgttaagactatgaattccatcaagagcaagcagggttccttagggcaccagttggtaagtgtaagaggcctcatccgcttgcaccagcagcagaaagaagctgcaggaagagtctcttttgggcatgagaagctaaaggaggctggtggttttctggttaactgcacaaggcctgcaagttctacagatgtcctttgaggaccagagcagcctcctttcctcccacactctccagacatgggaggcagtaggagttcaaagcacttctcagaaattgagcctttggttctgctgtcagcaaaaggctgtgaagctcagtccccgacactgggtaggagaagggtgaatgaagactagggggtctggtgaaggaagtggggaaacaaactcttcatttttaaagagagtgacctggcaaaacacagagaagatactttgggtttggaggaacgtgcaagaggtcgagagtcactgccgaggggttttgatgcggcactgtgctccggcaatgggaagggctgtggtcaggaaacaaagcagcaggtgtggcccatgtggaattgtcaggcaggatttacctgtggtggcccgtgccgcacagcctggcccaccttctgtccacctctgctttccttcttaggacctcctagaaagaaagctggaccttccgaaaagtagcttgggaggcaaataacatttatcgaatgcatcaatgtcttaggcctcaagcaagggtttaacatgagaatctctttgagttcccctagcatcctatgaggtagacactattaatatcccccaaggctcagagaagttaagtcacttaccaggaaacacaactattaaatcaggcctgcgtttgatcccagggatgttaaatttgaggcctgagctctttccatcatactgcgaagtctcaattgtggcatcactgcaaaagaaaaaccaactgaacgttgctgtcacatgcagagagagagacaaagaagagaaagggggatgcagctaaatgctgggctttttgctcttgcaggaccatgccacagagtccaccgtctggggagcagggcctgtgagctacagattctcgaacaatacaatcatgggtcaatgcctgggcagagaataggttctctttgaagttcattatctccagaaagcttttccagcttccagtcactgcttcactccagttttctttcttcttttttcatcttggcttattttagttggcttctatgcccatgactatcaccctcaaccaaagacagagaggctccaatgcaggaggcagacagatttgggcttgaacccaatgccgcttccttgctgtgtgagctttgtcaagttattctattgcctaatttttggggattgtattatgttatgttacatgttatgttttgttacatgatattgtgttatattactatcttcttcatggtgttattatattattaagattgatgagataatgtgtgtcagatttctagcaggtttcttggcatattgcaaccaccaatacactgaacatatactgtctatgtaactttctcagactcagagatttttgtctcttttatctcccacaattcttttcttttcaaaatgtcacttccagaattttttcccagaagaatccagtccattaaataagaatgttttttcactaaaattttctttctgcatactcttaggattgtatgatgttcgtctgtctcccccacctacaacctccagcagagagaagagggctgagccagtagtgacaagggtgggactttcacatctcttttattccccctgtagctttttgctaagggctaactgatttcacttaaaaaaaaatttttttttaagaggccttccatcctggtcagacttcacctttttgcaccttagtattctcggctagaaaatggaaataataagtcctcccccactcactttacagggtcatcataaaaactcgataagatatattgtttaaaatatttaaaaaccttggaatgcttcatgaatataaggcattgttattcaaatacagagcttcttaagagctttactttttagctctgtgatcagttaacttttgatgtggagcaggcaaccacgaattctcagaggcgtgcaacaataaacatttatttctcacacatctgtggagctgagctgatgtaggctgggcttgcttctccatctgggggtggtgtcgtttggctgaccgtgttgggcctggctgggttagtatggctccacatgtttctcatcctcctcccaggattagtgggcagcccaagcatgtctttcttcaaaatgatgctggatacataagagagcaagcaaaaacatacatggcttcttgaagcccaggcttggaattggtatgccattgcttttactggccaagcaagtcacatgggtggcatcagggatgggcttatgcatcatctactgtgggaggagttttctaagtatgatgagggcacagatacagggagcggtgaccattacgaacattaatgcgaatcctccacactgtgtttcttgatctgccaggacagggttctctgaaggccagtagaggttcagtgcttccaaaaaaggatgctgggctcaggtaggtagacagagttcatcacctctatcatgactgattggctctaaggcccaatgacaaaactctttacttctgtaaagtgttagacagtgaagaagatgaaaaggccatcatgaaattcagtctctgcttttagtttttgtttgaaattcggtctcattgttgtgtttgttttgttttgttttgtttttccatattttctcccaagggttattactttcacaacagtggaaaaatattccttatgttctctcccctctccttcccaggattcctttagattcctgtgtgtgcctgagggagctagaagagaaatacataaaacatataactgtgggactcttgtctgagcccatctccattcttaagtcccattcacccccacaaaagctttccttagagccaattatgttccttaggctaccatatccccataaacagacgtgagccactgtgggttcctagtaatgagattttgtttggcagttttatttgcaaatgactccaaagatctgagtaaaacatcttagagcaagaaattctgggttagaaattttcaaaccagaacacttcttcaaaggaagatgaggttgagagagggggtggcggtgaggcagggattgattttaatgggccagtatccagggctccagatgtttcttcaaaaggaatctcccaaattattgagcccaagttacagaataagcgagaaagaaaatggtctacagaatctttgattgggtccgcatgaattcattaaaagtggttgtcagggagatgtgaggggtggggaggcttgcacatatgtatattaacacacacacacacacacacacacgcacacgcacgcacacgtacacaaagagcttggtgtactggtcttgtcaaatgctttcctccctctgtccctccccacgaaattaaatccaaggtctggagttttgccctttaggttaaaaacaaaaaaaacaacaaaaaaaacaaactgctgcttttccaaaaagcattaaagagagaaaaaatataacagcagctactgacttgaattgaacttaaaatttcccaggaagaatctgctcagagccgaactcagaaacttgttgcaagctccccagacatgagaaaataaacttctattaagctggcactgaagtgcctggaagggtgttcaacccttagaacaactcttagctttccgaggagtttcagggttggaagaacaactttgccaattctaccctcaacttggaacaaagcattatacatctggaggagtctggtggtttaatgctttcttttccatccaagattattcattcattcaataaatatttagtgagaacatactacctactgggcacggtgctaggtgttgagatgtggtgatcagggtgaacaaaacagacatagtcattgtcctcctggaatttgcactttattggaggtaggggagcacaattaactaattacgtttacttgagataagtgtagttaaagaaacaaacaagatccagagacagaaagtagcaaggacttacaggatgatcagggaagtcttttccatgaaggtaacatttaaactacataatgctgatattttgaatctgaacctgaggatctcagaattttccacttctccctctgaaacagaggaggttttggtccacaggagaggctaagggttgattcagtcaaagtctttggagttcagattagccctcgcagttagctagaagtggggatggaaacttaatacacttgaattcacacacctccggaagtatttatggctacttctggttctgacggcaaggtcaggtgaatcaacttggtcttatgaatatagttgttttccatatcccttgttgaaaatagaagcaggtttgaatcaacgtaaagaggaagttgttaaacaccatgagaaacttccaaatcaggtgtgtcatggactctttctctacctgagataaagatgagtctgaataaaataactgagaagtggacttaggtgaaaatgagaagatagtaaagatgatttgtcacaaattgtagggaacaaaatccaacaccacactaagaaaataatatactatgacaaggtgggacctattacaagaatgccaattttgttcagttttagaaaatacattagaataatatacattagtataactcaggaaaaatattgtgattatctccagagatggtgaaaaagcctttgagcaaactgaacacctactcctaacaaaagcactcaagaatatacaaattaatggctgtttctttgacaagataagaaatatatagtttggtcttaacaccagtatctgtgctaatgagaaaatattaggggcatatttattaagatcagaaataaataatatcctctctcccctttatttgacattgtactggagatattagccaatgcaattaaacaaggtaaattaaaggtataaaaattagaaacaaataagttaaactgtcagtatttgcagatgatatgatagtatacatggaaaattcaatcaattataaaatgaactcaaacaataaaatattttgctaaatttagaaaagataagagaatttaaaattaatactcagaaatcaatagttcttatatacaaaaccgaacaccagttagaggatatgatagggaaaacctcatttaaaatccaaaggggagaactttaaagcttccttaatgaaacaaaagtagacctgaacaaacagaaagacaactttttgcttttttttttttttttgcttaggatgttttaatatcataaggttgccattgttctcttagctaacttataaattgactacaatcctaataatcacattaaaattttttatagtgaaattgatactgaagttatatggaaaaaataagaataactagagaaatattaagaaattaaaagagagctacaatgaaagattaactctgcctaaatttaaaaggcattctaaagtttctgtgattgaactagtaagaaccttgtgcgtaagtggaaagaccaactaatgaaataaaatatgatctgaaagagacccatatgcatgtgaaaatttagtatataataaaaatgcaagttctaatcacttgatagtcattcctttaaagagatacaatgaaatccataccttaaaccatataaaagaataaacctcaaatgtatgaaagatagtatatatagaaaataaaaccttataagtccacaattctagttagaagaaaatatcattaatttctgacttgatctgacctgggtctaactataatttggaatcctgtaataataaatgataatagtaatcaaaatgatgataaagaataaaatttgactaggtaaagtttagaaacaacaaatttgaatgttcaaaaatattatcagcagtaaagtcaaaagccaagtgacgaactggtagaaaatgtttctatatatatcagagataaaaagttaatgtccccaaagtataaagaatttttgaaattgagaataagcggaaaaccaaaaaccaatataaatgggcaaaagatacgttcatataatttaagtggtccctaaatatataaaaatatcttcaactttcaaatatccttaaacatataaacatacattcaaatatgtttcttgtaataagaggaatgaaaattaaatataatgagataccatttttcacctatcagaatggtaaaacaattttttttaaactcttaacacactgttgacaaagctgtgagaagaaatactctcagtgttaggaggaatgaaaaataatgcaactctttggagaggaagttgcaatctctaacacaaatacatatgtgtttctctcttgaggtggcaatccctattctagaaatttactctgaagctatacctctaacaatatgaatatgaaaatacatgtacacaaagttattcactgcagcaatgtttataattgcaaaatattagaaacaacctaaaatttccatatgtactagactgaatgtgcaatgatacatccactcaatggagtagtatggagttgtgaaacaaatgaggaagatctttatgcttgacacagagtgattttcaggatattttattgagtggaaaaagcaaagagcaaaagagtatttctgcaatgtatactatactacattttgtgtaaaaaagaatatatacaatgtatgctatgctacgttttgtagaagaggaagacagatgaaaatagacatatttacaatatctgtctaattttgctggcagaaacacaggaagaataaaccagaaatggatacaatttgtgatctatagaggatgtggggaaatgggatggaaaggacgagtggtgggcgtaggggacggcacttctctgaatataccttttgtatcatttttagttttggaaccatgctgacattttacctaccaagaaaaagcactaactatatcataaatgaagaacattaccacactgaactagggaaaaaatttaaaaactaatacaagtaacatttgaactcaattctttgactatagaccatcagactaaatacaaaaggaaacataaacattaaaatgtagttaacagtttttgtctaacacatagtgttagcaattcagaaaccactttatgtgaaacgtagaaatgagtgagtgagcaaatgcataactatattaaggataaaggagagtaggtttctgactgttgaagacaattaaaaatatgtacagagggccgggcacagtgactcatgcctgtaatccctgcactttgggaggctgaggcgggtggatctcttgaggtcaggagttcgagaccagccttgccaccatggtgaaactccatctctactaacagtataaaaatttagccggccgtgtggcacgtgcctgtaatcccagctattcaggaggctgaggaaggagaatcacttgaacccaggaagcggaggttacagtgagctgaggattgtgccattgcacttcagcctgggcaacagagtgagacttagtctcaaaaaaataaataaataaaataataacgtacagaggaaaatctcgaatgtaccaatagtaataatcttgggttaaaattgttggcatcagcaaaaactcatggttttattaaatatattaacatttaataaataatatgtattaatatataatttcatatatacgtacatctatatgtatcttatatgtatttcttttttatatataacatctgtgtgtgtgtgtgtgtgtgtgtgtgtgtgtgtgtgtacaccccttcagtcctatgaatagctgtgttcactgaaggaagcaatggcacttcaataaaaacgagcacatctagtcccaagaccttgtttctaagtaccattcctcaacaaaaagaatcctcaacaaaaagacatttcctgaagaaatgtctgatgccataggctggttggagcaaagaaggcctaacatgatcgtgaattaccttattgtaccagaaaaaaaaaaaaactatgaaaaaatgatggcgatatatcaaaaagactcaggatccagtgtgagcagctcttattgggcaagtcttggaaatttgaacaacaaaatcaacaacggttagttacacattaacatgtttgacaaaataagaatccatgagtccagactgatataaattaatgagtgaataaatgaataggtgattgagaagaaaggaaagcttctcttcacaatagaatgccaattaacaaatacaaaaggaacaatggagttagaaaagagttagaacactaccattttgcagcaattacaataataattagtttaggtaagaatcatgaatgggtgatacatttacggagactgtttgatgaggaactggatattacatagcctcacagtgtttctcataggctatttctaattctagaggaaaaatagtaacttcacagtaagaagcatggtggacaccaccttaaccaaatgattcaagttatcaataacaacgggacaatctgacatgtgtttcccaatatacagatgaaaaagagcatagcatcacaattttacaacataaatctaagcaaaaggaaagaaaagacaaacccaattgaaggagagccacattaagtaattggcttgaactcttcaaaatgcgaaggttatagaagtcaaaggaaagactgggttaatattcttctaacactgtaagtctcagcctaaatccaaaggcctgagaaccaggagtgcagacatccaagagcaggagaagagagatggcctggctcaagcagagagtcaatttgcccttccttcatctttttgttctattcaggccctggagggattaaatgatacccagccttcactggggaaggtaatcttcattactcagtctgctgattcaaatgttaatctcttgcagaaacaccctcacagtcacacccagaaataatgttttaacagctacctggtcatcccttacctcagtcaagtcgatacataattaaccatcatagagccagaacatgcaataacttggaaataattcagtcattgtgcaaggagttacctagactctgttttaggctggaaggtgggagtgagatataaaatgcatacagtgatcaggaggctacgagtactagaaagaactagaaagctttaactctttcatggcaataataaaattcaagattgttactaggaataaactgtaccagagttatgtgagacccaaacacagtcagaattagactttggaggtatttgttgaattgaactgaaccctatagctccccattgttcatagagtgcctacaacctgtggccactgcactaggcattcaggattcagaatgttgagaggactatgagaagtaacgtggctaagcatggaaccagagagagttggcccaaccctttcatttacaaatgaggaaactgatgcccacagtgagactggcctggcccaacattacacaaccagtttatggcaaagcaaagagaaacacctgtttgggtagtgagggttgccttttaggtgccctgaccccatttttaccaccaaggattgaggaggaattgcagtacaaagagacaagagatagaaaaaatttgagcgttgctgctgctatagaccctccatccagcaaagccttgagagagatattctaccaccgggctgggactgtaaactagttcaaccattgtggaagacagtgtggcgattcctcaaggatctagaactagaaataccgtttgacccagccatcccattactgggtatatacccaagggattataaatcatgctgctataaagacacatgcacacgtatgtttattgtggcactattcataatagtagacttggaaccaatccaaatgtccatcagtgatagactggattaagaaaatgtggcacatatacaccatggaataccatgcagccataaaaaaggatgagttcatgtcctttgtagggacatggatgaagctggaaaccatcattctgagcaaactattgcaaggacagaaaaccaaagaccacatgttcccactcataggtgggaattgaacattgagaacacctggacacagggtgggaaacatcacacactggggtctgtcgtggggtgggggaaggagtgagggatagcattaggagatacacctaatgtaaatgacgagttaatgggtgcagcacaccaacatggcacatgtgtacatatgtaacaaacctgcacatgtaccctagaacttaaagtataataataaaaataatatatattctaccaccgggctgctaacttggcaagggctgaaagaatccaaccgagaagaaaataggccatcattgtgcccctctccacatcaaaatgcaattactgagcttttcttaagagctcagcactgtgctagatgttgtccatgttgatcttcacaactgcagaggctgaattactatatgtttatgtgataactaaatattctattatgcttgcatttgtctttggtgaatattattgaagtcgtttactgctacggtttcctctgcagtttttctactagtccatcacactcttctttttggccctatttttacccatactcagttagtttcttgtggcaggctacgtgtgtgtgaggttctgtgtgtatgtgtgtttgcgcaaacatggttttgtgtcagtatacctcatagatatgaaaacagtttctgttcaaacagtacccacacttagactccatattaatcttggctcaattctgtgaaaactggccctgaagtctgtgaccccccttactcacctctttacctcatctcaaccaaaagaatccacttcaagaacaccttccgcacaagctcttcaaatatctcaagttctcttttccagtatggtgttgcagggtgtagtgaaaaatgcatgagtttggagtcaaacagattcccgttccatttctggttgtatcctatattaccacttggagttgttggcagtcttaggtgaacacaggaggcaattctttttttacagtgactgacaaagaggtgagcaagaaatgtgagtctcttatttattcccagcccctctatcttaacttgttgaaggtaaagaccagtggatctgaatttagattatttaccctcccatgtgtggggaacacacacacgtgcacacacacaccaggctggaatttcttcagtttgttcctccgggtctactctatacttcatggaagctgatcttatgacttacatcaatggtctcccttgtcctctggcttctattagatttggcaaatggggagccctggcagatcactgaagggaggacagaagttgtagtatttattcctgtgatattctctcctggtgtgtcaccttgggctgtctgcgtcccttgacagaaggtcaccctcttgtcaggcatcacttttctcacagttctctctgtctctgggttccaacaccactccctcttcttgacccttaacgcctaggggtgattatagctccagctgttattagcacggaggactgcacgatcccttatgatttcactacacccagtaaatagaacctttattaagcttttcccaaataacccaatttcagtgcactgtctgttttctgcttggactttcactcacgtatgtgcatgcacacacacacacacatacacacacgcatgcacactcatacccacgtactctctatatacgtgtacattgagtgctatggaatttttagggttctttgaggacaacttcctggaagaagaataatgggagatgtgctcagaggctggtgagcgtttaattgacctggttgtgagcacaatgggaatgcttttctgaaccttccatttgcctccaatttagaataactgcttagtcagcaacatgtctccaattaacatgacagattttttggaccacagatctctttgggaatcttatgtaagctgtgaaacttctcccttaaaaaacctacatatttaattctgtctgtaattttaggacttcacatatccccaaagaccattcatgatccaccagggttaaagacttgttttaagtaaagaaaatattcacagcctctggagtagtagacatctccagagaaagtctggcattctttggtaactcatggcatttattacattagctttggatggaacaacccccaacccccaagtctgagcatgtggtttgatgctgcttcctttccattttcactctgctctcttggattctatgtagcaaaaaggtaccttttctagggtcacatgtccacttctcaaactctgtcttgcactggggatttgagggtggactatcagagcataatggaagaggcaggaaaggatagcactaattagagaacttgataggccttttgctactgataaagattctgtcctgtatcatccatgaggtttggatttgcacttctaaaaaatagctgtgtgtttttgacaatagccactccctttttgctcctttctggtctctttcctaaatgactttccaggatagtataaaataagcaatctatttagaggaacaaaacaaagaactacaccattagcattaaaatatttctagggtagtttaggtaacatagaactttgtagctcagcagtcttggaatcactagactctaagccccatgtagtcagagactgcctgcttttataattcatccccaacagcaggctattgcataacacattgttaccacccacaccattttgttgagtaaatgaatgcatgcatgaacgaatcagaggattctgttgtctaaataaactttggaggtgacttagtcttctcttccctctgatgcttagatcttctttatctctcagttcaacatatattgctcaaatggttcctaggtgcaagggatctggaagttattaaaatgaaatagattttggaccatgaccttgacaaattatgatctactaggagagataatatggaaatcagtataatactacagagacagtaaaaaggaaaaagaaatgtttgcacttaataattattccaccattgttccaaaagtttcaattatagggaactaagttccactactaataaaagtcaatacgcattgtatgcttactgcatgctaaatatattatgtcattataatatttatgttcaggtgtatactatgattgtatgtattttacgacaaggaaattgaggctcagagaggttaaagaacttgctcaaggtgacagagccagtaggtggtagaacaaagactcctacattctgacctagtccatatgtaagcagatttattgctagaaatatcttgatgttgcattgaaatctgcctctccttattttccatttggaactaattccactctcccgagccagaaaaaccaagcctctcattacttgaaaaaccaattaatgagttaaattacatcaagttagtttaacagtcagcaattccctgttgtaatcatagttaggctgctaccatttttgtcccaatcattggcccacactgaattttgtgtctaattcattgctttctgtctccttaatttcatcctctgcccccagagatactatgctgagtattgatgctgcagcaacatattaatttttatgttttctgactcatttaccccaaagccatagtgcatcaaatgcttttgggtttgtgaccattttatctttggtttcagtcactgtttatctggtcgcatctttgttcatttaacaaatatttgctatgaagtgcaagtttagtgggtagcaaacactgctctggcaaggggtatatgaagaaagacatcattccttcctccaagactagtcttgtttggaagctggacaaacaaacagttacaatacaattaaaatagggttaattaagaaatgtgccaactgtcaagggtaatcaaaggggaccctttcagtctgtcccaaggatgctaggtaagtcttcacagagaaactggtattagggctaagatctagcggaggactaggggctttctagacagacaatggaggcaggacacttctgagagaggaatgaagccttgaggtgtgagacagcatgtggtagagcatggagggttcaggcacttggaggagttgggtatggggagaaatacagagcacctgtgagagcctggggcatggctggagaggtaggcaggggccggcttgcaaagggctttacctgtcatgctgaagggctaactgctgttctggagaatttgcagagccactgaaggttttgaagcatcctaatgttaggattcacttcaagttttagaaagtttagtcttttggctcaagggtggggagggaataaaagaagcgagagtgaaggcatgggaccagtttagagactagtgaagtctgatgaaaaggagatgagagaagcctaaagtggacaagcagctggacacggcggctcacacctgtaatcccagcaatttgggaggacgaggtgggcggatcacctgtagtcgggagttcaaggccagcctggccaacatggtgaaaccccgtctctactaaaaatacaaaattagctgggtgtggtggcagacacgtgtaatcccacctactcaggaggctgaagcgggagaatcgcttgaacctgggaggcggaggttgcagtgagctgagttggcaccactgcaccccagcctgggcaaaaagagtgaaactctgtctcataataaataaatagagtggacaaggaaatactgatgggggtgagaaaacaatttgagagacatttgagaagtagtgagataacatttggttactgattgaatatggagtgttaggataaccctgggatcctcattttgggcaatcacaaagctactggctacagcaaaaggaggggaagaaaagtggtggtgggagggaggcatgagaaatttgctgctggccatgttaagctccacctcctgtcaccaagtggtgatgctcaggagatgtttgtgtccacaggaggagagtgatctgggtgggaggtttacatgtccctccttccatctcccggtccttctttccctcctccctctttctttgtttggtacttcctttcttctctcttttctttttctgccttcttttgagaatgaaacctaagagtgtttatatgtagagggaaaaagtggagctagaagtttaagacacaggaaagggaggaaaaatgtcctagtgtgagattccaaggaggaagaagggccaggataaggtggggagagggatgcctcttttgctcattgcctttatctgtctttgtctgtcctggattgattgtcctatccttttcttccacttcgtgtcttctcccgtatttctacacaaggcacagtcaaataagggctggcacgtgcgcatctttgtgcacacatgtgtgtgcacaagcattgagggatgcacaggtgcacacaccttgtagagcctctagtagctcaagtcacattttttcgcatttccagattgtaagccttactgaggggcagatgggacgttctcacaggaaaaaggctgacctcagggagaaagatgtggcccccacctacaaaccaatgatgtctactcccaacaggggacgtctttcccctgcgtgaagcaacaggacagatttggttaggtgtggcatcgatgtcctaatctcctatgagcacacaatgaatgaaggtggggggctctgttaactggagggaatcaggggctgccctgcaaagcatgtgcaaagaaaataaatgcaattggagtgttgtctacacatcgtcctcagtgggcacaaaagaggcacagtgggatggggctctgacccagtttccatttggccagccgactggaaacagctactcactgggccactgggttctttccaaagaacagtgaagaacagtggtttggggtaggtgggggcaggggctggtggggcaagaatgtcagaagcaaatgcaggcgtggctggtccagtagttccagtaccaaagaagccgctatctggaagagtgggacaacccatcctaaacatgggaagaagaaacaggaaatttcaaaagatacaactactaattaaatgaaagcactccttaaccacccctttgataattagatcatcttctcctagaatgagaaggaatattagtctgactttgccctccatatggagaaacatggcaggcaggctctctgtgcctggagggtctggccagactttgtctggcatgggtattttgactccagcctattttttagggatcctcctgcagctggtcctcaagaggtctgaactttgccgtctcatttctaaagcctctttcagcaggtactggggttcccaggtccagggaagcctggtcattgctagaacacatggcaacatccttcaggaaactcagctcagactgtctggaaagatgctggaaaggctgaatccatgcaagtattatctaaccagccccaagtcatgttttcacacttagatttctacaggatattcataaattctggaaacaaagatattgccactttattttatttttacacattgagaagtttccttggcaacataacatgtatttgcctatgtttccaaggtttatggatgaatgtgggtggtcaatgggattcattcattttgccaagcaaacgatatgtttgaagccatattaagtgccaggtaccatagaggatacagatgttcataatgtaaactgccctctagcagcttacagatactacctaggaataatgcttacactgcagtaaacaacataaggcagaatatatatatatgtctttgtactcataacaaaatgtaaaagtcaaagtcagggctcttttaactttcttagtagcagaaacccaaattgaacttaagcagaaggggaattatttatttaaaacacaaagaaattatttattgaaagcaaacaggaaattgattattgcttcttacaactcatgagctaaaagacagagctgggttcagatacagatatatccagagttgtgatgctatcaggacgctgtctggtaatgaaaccaaccacagcacagagcctggagtggagggacagaccaactctcttcagatgccacatcctcacccagcagacctcctcctctccagcacccattcatcaaaccttcgagaagaccctggttgctactgcaaggctttcagcaccaaatgggaaccaactcctcctccttggacatggaatacttgaactggccattctgggacaggggcctgcccatgtgaatggagggaggaagaagtgaaatccttaaagggttgctgggtcagcatatcacaaatttcccttgccgtgagccagatctgatgggatggtattagtcaataggtagataatagggggaagagggggtgaagaatgagatggttctacaaaaagaaaggcagggtgccagaaggtagaatttgggcaatagtgagtcacagaattgattgtgtcttggagcccatggaagggagctgaggcacctacggttggagggggaggtggggacccttgggcacagctaaagagtctgcttttgggtaggatgtacagagcaattagaattatttagcagtgacttaaagccaagagaacaaatctggcttcagagtgttgacatggaagaaaggaagaggcaaatggaagaggctattccaaagggagaaggtctgagcttggtggtgcctgggaaggaaggacactgcagccaagctgagcctgtgtccagctgaacacggacatttacttctgcttctgaggggaaatattgtcatgtcttatgggtgtacccagcaaaagatgtttccactaaaagactatagagaattgcttatttctaaggactgaattccgaatgtttttggtcaggttctctctgaacagagaaccattgcccctgagcagaagcggtcttcacacaactagcaaccatgtagtgttttctcattcaaatggcgtttggtattcacctgcctgtcctggaacaaatgcaaagtcagggttgtttctagctctcccccaatattggaaaaatgctgatgaactgagcctggttccattttacacctgacaaaaggaactcagatgagagtgttggtaactgggtttaaggaggagtcagaggtgactgctgaaatttctgccagactacaactaaattaaaaaaggaattcggtttaagaagccaatcttcaactaatttgaacatgaggatgctcaacttagaagcctcccatggggccctggagcccagaagggactgcatactctttgctcctgcaggtaactcagggctttgttctggtgtctctgacaggcttctaaccttctgcaattctgttgccacaatttctgtggcaccaggagaaaattcatgcatctaggctagggtggtgctctcatgtgtctccaggagagtgtggcctgggttagcttaagactaggaatgtgctgacagctgggaagaaagtcaaaagaaggcatggcatgtggttatggggccggagggaccgcttggggggagaggtggaagcgctgacatgagcttgctctgcatcgctggttcccagaggtggcctgataggagagtgccagctggtggtatgccgtgggctctggggaaggggctagaatgttgttgcagcccagaggggctgggctagtgaggagcaaaagaaaacataagctggggggaaactgcctaatagaccaattaggagacagggttgtgccctaagggatgggtgggagcagggggtgctagaagtagacagagagtaactctcagctctgaacagcgtcccacataggaggaagaacactgatgtgaggccaagtggcttggaagggcgttgacaccccattccctacaacaaagggtttggcaaactcatctcatttgctacttgtcctataggtaaggatggacaccaaacccaaagagacttttgggatgtggataaaaattgaaaactttgcctctcattaatcccttggagaatatagcatctccttcaaactggactgctctccttgatcccttgtctttacctgaaaatatctttcaaggcctagctcatgctatcttggccataggaactttccttatcacctccatcaatgctgtttgtccctccactagaatcttctgttttggtactcctcttactcagcctcaagttaaatttatgtgtactggttttaaccccagtacccagtaaaggttctgaatacacgtgatgaaataagtctctcctatcccatggcacctagtatagcatttagttgctatactaattattataattagcatttattacgggcttactatgtgcaaagtgccattattccatttattattcacaaaaatatttagtctctactattctctaatttattagataacctaatataaatcttttttgttttgattgttgctagctgattgaaggaaatagaattgtggtgtggtagtaggggatgttgagtgagtcttcctcattcttaatatttccttccactaggataacgcccctgggctctcttcatcaggggaggaggagtgacttcatctgatagattcggaagggaaaatgttacccagtcgcttctcagtcctctttctcttggagcgagcccatctctggagcacatacactctcccctgctcctgtaggtctcgagttctcaggggaagggtttgccctgtaagagagcttgtccttctccagtgaggcctgtcactgggaagataattctgtacagtgctgggattcaaatttagaaagcccacttgccccataattaagccacattcaccagctttaacaatgacagaggtgatattgagttctccagtgatattgagttctccatctgccttatgcttttctgcttatttccccattggttcagaattctggtgggaaaaggggtgctgtttgtgggatgccttcccaccctcatgtggccctccataagttcttcatccctcattgcattcaataacttgggtgtggtgtgggtggagtgcatgtgccatatgaccctctcccttgagtctttgaaacctatttcttactttatgactttggacttccttttctttgggagtataaaagtggataccaccattccaccccttacccgtcatcaggagaagttagttgggaagcaaaggcggagtgacgtggagggaagagtctaaaaacagctctacacttagctttgtccttgcattgatgacaagcgatgttggatcctgaaaggtggggatgggaggtaggaagacccaggaagttgctgtgaaggctaaatcctaaagacagtcactaaaagtttccagaaggggcatctatcactccacaggcaagcaagatgagaccatgttgaaaaggtcttaaagaagccagggaagaggtgaaacatctctctgtgtgctgaggaccataccaggtccttcattatcatattctacacattgaaaatgaaggtaagagagcaagcaacttgccctaggctacccagggaatagggaggaaagtctggatttgaacctactgctgcctgactccgaagccctgttctttctctcaccctggaggatggatgagggtgggatggggcagttaaaagttgaggggtatctctaggagaaagatgagacacatgaccattgggtctaaggagccagagagaggccattgtgtctgagcctggacagccaagcttcctagagacagaggatgggatgagatgacttctggctgactctgcctccttgagctctttaggatcccctgcagaatctcccaaaatgatgctcattggaaacagagagagcacattcaagtaagtatttgtgccacactatgattgttccaatgacgtcaagtctgggatggcaacagcagcggttccatttcttttcttcaggacctggattcttgctccaaggtcttccacctccctcctcattttcttccactcccactgcaggctccttccttggaggagctggggcagggagtaggggtggaggttgtgtgtgcacttgtgcacatgcatgcctgtctcttatgtgttgctgagtcaggaatgggcaggaaggagaggcttggacagcaaggcagccctgggcaatctggtgatgaacttgaaacgggagagggtctattgtgcatttgcactgactctgtgcaggaagggtgggggcaggccaaggaggactggcttggctgagacatgagatgtctcaggggagggtttggctgaggcagtggaagaatgcttgtcgggaaccggcctgcctggaattgtaacctcccctgctgtctctgcaccacactcagagtacagctgtatgcctcttagctggtagattccttttctagctggggacaccgtggacagggacagtggggaagagcattggactcagagacaacttgacatcaagtcctgcttggaatcctatttgggtttgtaattcgttgaaggcagacacagacataaactaatccttagttttcccatcccttcaataaggagtacaacacctatcacacaaggctacaagaataaaagagagaacttgtacaaaggcagggcctccacttggcacatcttagatcctagacaaatgctgaacaaggcagcatggtgtgatggtgaagggttacagccagactgactgactggtcctgtcttcgtttgaagttttgatattttgttcctcgtgggttttttgcatttttactatttatttatttatttatttatttatttattgagagacagagttttactcttgtttcccaggctggagtgcaatggcgtgatctcggctcactgcaacctccgcctcccaggttcaagccattctcctgcctcagcctcctgagtagctgggattacaggcatgtgccaccacatccggctaattttgtatttttagtagagatggggtttctccatgttggtcagcctgttctcaaactcccgacctcagatgatccacctgcctcagcctcccaaagtgctgggattacaggtgtgagccaccacacccggccaatttttttttttttaaaatatggcattaaaatggtatttatcttgactactgagtttttggttaaccccttacattttgtgcccaagacatctcactcatgttatcctaggcccagcatggctggccttgtctcttaaattcaaatttttaaatttttcattcctcaggcttctcacttgtaaaatagaaataataatagcatctcccccacaggcctgtgctgagagtgaaatgtgttactacacgtgcctcaccttcattttcaacaggacctattgccagtaagtgctcgttaatgttagccattattcttggcattgctgttgttattagtgtcagccatcattctctcagccaagagatccttccagaagagaagttctccatggagggggatgggagatgagctgaaccctggaaacagaaatggatcagatcagggagcctcagtgccctgaagccagggcaggggtgcatggggcttctgcaaaacccagggccaagcatgcaggcaagaggcagccttgccaagcccttgcagaaaatgcttgcagtgcaactcctctctggagacgccacaagttgccccgacagcttttccttttggggcttgtttaacatttcaaacaaaaataaaataaataactgcagtgtttatggaaacctaagacagaaatgaagacgacttagaagaaagttaagtggaagtgggccaggagggggtttagaagatgcccctttccttggtgttagcagttttgtgcttcagtttgtcagctcttcctactgctctcccctagcaactcctctgccagtgtgcacaaatgcatgtgtgcacacatgtgcatgtgaatgcatgtacacacacatgacacacacacactcatcccaacgagaggcacacatttcagacaccatctcacacacacacatttctctctttctggaaatgtgtccacccactttctcctttatcttcttcactgtgtttttttgtttttttttttttttctttttagacaaagaatctaattaactcattctggccagatggtgtacattactttaactccttcactcctgtgaaaatttttggcctcaaccttcactctgaatagaccaagccaaactcgttttatgtggataagccatttccaagtcaaagttccgtggcaggaagtgggagagtgggcagagggagcaggtccttacaagcaaagacttgtataatgctttgcctagatttgcttatcagtatgttaaatatatgtggactattttattcttccaccttttattttggtatcttctttgtctatccagtctcactttgccagactccattatgcaagagaggctttgccagaagcaaggatgctgtagaggaaagtaatgaaaatgtgaggatttctcagaaggccacatacaatctctaggtctttccagtacacacagcttcaaaagccaggtgctgtgttccccaagcaggtcagggcatttccaggagccctggattccagaagctcaggacacctcagtggtaaagagaccctaatgccggtgacagcacagaccctggaaaccctagattgcctccgtgtgttcctcttggcctcagcagggctggtttccccctcctgtaggagcagaggaggctctcctctcttgagcccagggcctagcatagtgaccataaggaagcatagaagatccagctgcagatttggaaaacagagccctcatctctggaagagccaaaactgatcctcaacatgtgccatgcacatcagatatcaaacgcttcattcattttgccttcgctgggtaaccagtgaccttgagaagtcacctctcttgagtctcaatttcttcaagttgggaacattacatgacaggacaggactgtgaggattaaatgtaaacaacattttttttttttctatagaacatactgtggggccagcaccatgcaaggtgcttttatctacaatttatttctttcccttcccatgccttctgcctctttgaataactgctgcaatggctgtagagagataaatgcagtagaccaagacttttagtgatttttaaacaatgttctagggcaaatacatgggtatgtttacatactccacaaagtgaaaaaattatgctagtcagtgtgcaaagtgtgtttttccagacctgcaaaaactgagatcaacttccatatttccaacttccatattcccttcatcccttaagctaccaggcagaggatgggagataaaatttggccataaacttgtcatgggtccatcttggtcctaattagaattatctacttcctaatccattaactgcatggatagatagtctgagggcctcaagggcaagagatgacttctacccctccacccatccagccctgacccacccttctcatatacattggagaaagctctgctctgtgtaaaacacatcaagtgcaggacatggttcttttcttcaactatctacagaaatgaatagcggccctagagaaatgcaaaagatgcctgcaatccaaggccaaattggagaacatttcaaggagaactgggcctttattgtagagtttgggaaatgagtaacatgtagatctgtgaatagatatgtctgaggaagcaccaggcagcggaaatagctggttgcaggagcagtttgtcctgcccacctgaatcttcaggttccttcagctaagccttaggagaggaggctggagagctgctttgctggatcatctcctttggcccccctcagactagaactgcacctcctctctacctagctctgtgctccacaggctgcctctgggctgcactgatgggctcccttgactgctggctcctgttcagggttggaggaggaggatgaagtcatggaatagtgtccccatgctgggctgcaggtacagttgctgcttctctactgaagacccctgacagcaaccctgtcttacagccagggtgtttgtagttgctccctcctccctgtccctttagagtggtgatggcactatatgttagatatggtcagagcaggagacatcattttcccttaattctattctgccttgtaacccctttcttagactctctgctgagtctctgcttatgtgttgggattgcattgatgcagatgctttagggccacgttgtggaggatacaagatgatgagacaggaggcagcgtatggtgaatcacttctcacagctcctaactaagttctgggcacatgaatgggtactcagcacatacctactgaccaagcctatggagagctgaaaactcataggctgactttgaagcgaggcctttgaaaccatcgtcccaggggaaataatctaggtcaaagagacggctgtggcaagggtgcgagctggcaagcatgagggctgacaaagcaggcagcactcctcctgagcagatgaccagccccgcagtgctggatgggtacgactattccaaaccctggtgtaattctgtcctctaacgtggctggtttgactcagcactgctggggagccacagagccttcttctttagcatttatgagctcattataagcatccaagaaaacatcaacaaagggtgctaagtccctctagaactgccttttaaatttttcttttgtaaaaagaatgatgctttaagtaaaaccacaacttacagaattcagacaaaaccaggcagcccccaatgttgttatcccagctcctcttggaataaatttgggaatgaacaaaaagtcacagaagagaaaactccagcccacacccacaccttttctgacatagccgcctgtgcaggtttttgggaccaggtctgccatctgtattcccaggctgggctggagcacctgaggctactatggttacagaaaggaacctggcatccgaaggtataggctcaaatcccagctctcatgattgctagctttgaggtttcaggttcatttcttaagtgctctgagctttggtgccctcctcttcaaaatgggaatgaggagcatgtctagctccctgttttgttgcagtgaatagctacatgaagaacagctaaaccagaacagaaagaaggcaaatggaagctacagaaaccaaggatttccttgttgaatcgaatcttccttcaatcttccttcaccacactagtggatctccctgtgggagggatgttgagagtgctccgtgttttttttgttttttttttttttttttttgtgatggagtctcactctgtcacccagactggaatgcagtggtgcgatctcggctcactgcaacctctgcctcccaggttcaagtgattctcctgcctcagcttcccaagtagccaggactacaggcatatgccatcacacccagctaatttttgtatttttagtagagacggggtttcaccatgttggccaggatggtctcgatttcttgaccttgtgatctgcgcacctctgcctcccaaagtgctgggattacaggtgttagccaccacaccagccttctctgtgtgtttttaatcaatagcaatatgtaccatatttagtagggactataaagaaattgtgctagaaacacctggttattttacagaactttgccagtgagtacaaaaatatgcgtagcccacaacctctctcccctagaaaatacctcctgtgggtagaatgaaatgggtaggtgttcttaccattcattagcatttcaacctggctatcttcttcctagaattccactactcccaattgcccatgtgtttccaataaaaccaattcaattgtgtaccaacttggtgacaaaacctttatgtagttcttctatcattttgtggggttttcaaagagggttgctctttgatatggtttgatgctgtgtccccacccaaatctcatctctaattgtaatctccatgtgtcaggggaggggcctggtaggaggtgattgaatcatgggggggggactgttcttgtgatagtgagtgagtactcacgagatctggttgtttgaaagtctgtggcacttccccctttgctctgtctccctctttctctcctgccaccatgtaagacgtgccttgcttccccttcaccttctgccatgattataagtttcctgaggcctccccagccatgcagaacagtgtcaattaaaccccttccttatacattacccagtctcagctagttctttatagcagtgtgaaaacagactaacacactcttaaaagtaaatgttattacccaccttttacagattaagaaacttaatattacatcaaggaattagtttatagcactggatgatctggcctcaaatctccagtgtgatcagtatcaagcttttctcactgtatcaacagagttttcctaacaaatcataagacacaagtagtttctccaacttaatctattatacccataattcacttgcctccagggaaaaaaattagatgaaacagactgtagtggctttgtgtaaaacagcagctccattcctttagccctattctaaaacaatagaccatgtgctgatagctagctaacctccattcagtgcttcactatgtgtcaggcatttgttccaaacacattttacatggattatcccatttaatctttacactaaagtataatactcttattaccccattttaaagaagagaaaactgaggcacagtgagattaaggaattcatccagtttctgctgcaagtagggtggctagcattgtaatctgggcatttgccatgaagctgtgcccttaaccactccactatgtgctataaggtggagctgacttcatgggggctaattcaaccaggggagtgtgctcatattaacagatctgtgaactccctggtttgtttgtgagacaattatctatttgcttacctgaatcacagcaataatagttatacctcccacttactgcatgcctacttggtagcatgtacctggacagaaggttttcatttttatgacaaacctgtaaaaaatggcttcttagcctggtttttcagatgaggagtttgattctccatccaaagtcatatgataaagaagtggtgaaacaggaattccaacccaagtttgttttgttttgtttctgaaatccatagactggagtagaatctcaaattatatgacatttgtctatgcttattttttagtttccaaggtgatggattatttaaaaaccaaacgattttgaatgtaaaacaagcttaattcaatttacttataaacaatgagcactgactcttgtatacatttaagttatgagacactccaatattaattcgacccacacttttctttctataatctattgctcccaaatattctatactatctgcatgtctgccttagatacataaggatccagccctttcccatgtgtctctcaggatggcacctttgctctgggccggatgccatggggttctcagtgatgcgcactgaataatgcaaatatgaggacataatcacagaaggaagcccttgaaattctaaagataaggcccacagaccagggtactttgaataagtcagtcatccttttccaaggttatactttttccccctaaaataataactgcagacacagttctcaaagcaaacaggtctggtatagtagaggcatttttagacaatttttttctatctacttgttaagcacattttaaaatactgattgccaataaagggctttccatagaactcaaaattccaagtctctgtcccaagcttctgggagcattcctccatttttctgtttttattttttgagatggagcctcgctcttgctcaggctggactgcagtggagcgatctcggctcactgcaacctctgcctcctaggttcaagagattctcccacctcagcctcctgagtagctgggattacaggtgcatgccaccatgctcggctaatttttgtatttttagtagagatggggtttcactacgtaggtcaggctggtctagaactcctgacctcaagtgatccactcaccttggcctcccaaagtgctgggattacaggcgtgagccaccccggctggtcgcattcctctatttagtgttaataccctaagactccccagctgaccaccaaatgtaggcaggtggagggactgacctccagggggcacatgtctcaggttgacaaacacattttctcttgatcctttttttcagtatgatgaagtattctacaagactctcattgaaccttctgatatcacattctaaccatttatataaaagtcaaaagctccttgtcttcaggattccaaagttcttagcacaaaggtccaggaagtgtctgaattctttggtgtctggttcagtatggatgttcgggtccctcttagacttgagatagaacaggaaagagatgaatagacaatgacagaggtagagaaagtaccaacccttcccaggctgtcggcttatttgactgtgggaatcaataaaaagtatttagtgactaaaatagaatttgaacccagttcagatctgtaagctaacattttttaggcaacaccatacagcctcttcatcattccctggcctctttcatcctatccctttttctcccccttcctctttctctcctttatttctccttaatcatcttgcttctctttcttatatcttctcagtaagtaaataatccacaacctcacaggtaaattaaagttgccattcgcacatctgttggctcccaggtatggttccaggatactgggaatgttaggtagtgagaaagattcagatgatattaatttcctgcaaatggaatattcatggcaggcacagtctggccaattgctcatggcctggcgagtacgactagcctcgattgtacctggtctttgcgaatgtggtggcatgttcactggcatcacatactccctctcatctcctgaatggctgaccagtgcttgcttcaagttagattagagtttgagctttgtaacataagtccccttctgtctgatcaatgaaatgagggataggtacttgcccgagagttatgggacctggcatttaacgtgagaacatgcatcttcagaagcactggtgagacttcagaattcctgaaagcactattcttgcaaggcttgaatgtaccttctccactccaccatgtcctctcccaaccctactagcatgatatcaagcttgggtggagagctcgcttgcaactttgtcagatacccatgagccactctttgaatttctgacaatgaacaagtatgtgggaggccaaccccatccaacctgacccatctgaacccaatcacatgttttctattttgaaataaacaggcagggtctcaggaattttaagtgagtcctctatatctttctctatcttccttttcttccactaattattacagaattgatctgccagtaattactactaaattgaatttcaggggcagtgctgtatctggccccaataccctaagtccagtaatagcctggaatgttgctgagttgttaccgtccttaaagggtgaagccacaatgatactttctataacttctatttggtctttttaagcccagcacttagaaggattcacttgtggagaccttttcatttctttcaactctacagcttagtgcctgaaaggcacatattggtctctgtgaatctctacgttaagaatctgatttgcagcactggaaaccttgctagtcttagctctgggctgaatcccatgaagccacgccatgcctagggctttcatgctgtagtccatacctctgaagaaaacggagagaaacatcttcctcccttcttccccccaagtgtgtgtctgttctcgtcagattcttggcagggttaccaactctcttctaagcaacttggattcacaacagaatgattccttttcaatgccaatctaaacaaatgccatcaactcttatcccagggagtatgaaaaacagaagagtcctttgtgactctgattggctttgtgacttccttggggctggtgttggtctcaggcttgtgtctcgcagtcatccaaaatgtgggtgggccatgtcccaccttaactgtggttgtgaacgcagaggattcactcaactttcagcatgcaacaataatcaaatgcagttttatggttcacagagggctccaccttcatgatttcatttgatcctcctaacaaccctgggagctcagagagactaaggaacttgtctcatatcacatacccaggagttgactgcacggcatctgatgccagcttctgtaccacgtgtcctcctggtgatactagagtaaataaaacataactagacaagtaaatattttgtacaaagtaatctgctaaaaatgtggatggcattttaggttatttgttggtttgggttatgcctcttcttattttgtagataactgaaagctaagagcattgtcttatttatttatttatttatttaggcagagtctcactctctcacccaggctggagcgcagtggtgcgatctcagctcactgcaacctccacctcccaggttcaaggattctcctgtctcagcctcccaggtagctgggattacaggcccctgccaccacacctggctaatttttctatttttagtagagacagggtttcaccatgttggccaggctggtctcgaactcccgacctcaggtgatccacctgcttcggcctcccaaagtgctgggattacagcactttgcaatcccatgcgtgagccaccgcacccggcccattgtctgtcttcataagcagaaggctgtggcacagggtaggagaagaacatgaagacaaagttgaggggagttgcttatctcatgcttcttgctgctctgcctgaagcaaaaatgctttatggatacttcacttatgatttaaaataatagctcacattgtgtacttactgtgtgccaggaactgtgctaattctttatattatgcccataaaacctgcttctgaagcattggggggtttgcacttgccgttccctctgtctggaatgcctccctatgcctaccttcatgaacctcttccctttactttccccatctctgttcaggtgtcacatttttagttattctttcctgaccgctcagtataaagtagctcacacctctacgttatcacactccctcttacctgctctatttgtctttatagccctgctcacaattatagtgtacatggatttcttaatgtgtgctttatcttccccctcccatcggaatataaactccttgagagcaaggatgatattttcccctgctgttttctcagtaccaagaagagtgcctggcacgtagtagctgttcaacaaatatttgctgtgtaaattatctcattttatagataaggaatctgaggttctaagggagtaaatcattgacttcaggggacacaacaggcaagaggtggaacacagtcatgagctaaggcagtcccagatcctgcctgacccttcactctcttcctcattcagtgaatcgtaaggcacttgggctgtttgagcagtgcccttatccataccctgcccagtcagggcctcccagaaccaggtaaaaggatctggccccctctgccatcctcccactggtgcccttcccctcccacccaccgcaacccccatctagggagaggaaactctttttccacctaaattccccctctgtctagttcatccaagtcatctcccttcccaccacactgtttacacttgtgggtctgtgttgatgcatatcacagcccccttagtcattgtttagccattatactttgcaggggtaattttccatccctctgtttatatttttgcttctccttcatgtgctccccctcccacttcttaagggcaaaggaagacatgttcagtcatacaagcaataatagagttataattaagatgggaacattttgccaaaaggaggatttagttgtgatttgttgaatgccatcctgagtgcattatttaagagcaaattcaagctctttcatgagacttcctgaacagtgcagtcctctgagcctgctgtctctatagtctttgtgtcagctttgtggggtcaggattgcgcgtgaaggatacatgtttagcaaggcctggttgctgaaaaagcttaaaaggaaacttcagggactatctaggaagatcttctgtgatctgttctctgattttgtgtgctcttttgtggttagccattgctcttggtaccgtgtgggacttagcaccctgtgcctcaaggtaagaacacacttccagcccagcttcagtttaatgttcactatgagagtctccagtaacaacccacactctgcgtgggattcactgcctttcccatgtcctggtgttggacatacatactggacagagttactgccttttcccctagtaattaggggaaaaggaccacattttcccctagtaataggggaaaaggtgtccagtatcagagctaggtagagctgaatttggatatgaacttgagctgtaccccatggtgtcttttcttgcagtcctagcctccagcgcctttagaatttgcattctgtttggtgggatgaggacggtggcagtggagacagggtgagaggagcatggatgggtcctaagccttcctgtcttcagcagcatctcgtgctctgcggctctgctgtgtgcctcaggggctgcccattttcaggttgcatttattagcttcctggatgctctggcttccagtagggtttagcctgtgggaggcactggatggagatgggagggagggagagaggacaagagaagtcatgttatttctcctttgccttcccagcctgggaggtttctccagtagcagctgcctctcttcttggattccagaccccactagacaggtgtgctgtggctccagcttctgtctggtgttgctggcccctggttcacctgactccagctcctcctcctccatgcagctgggtcctgctgctgcctccctctgctctgccttgctgtcccctgtcaggcctctcagctcctccatcccctgggttaccaactcgctatactgaaatccttctgttttagaaactcatatgtgattttcattcttcttggttggacactgactgatattgagggatttgtcttagaagtaaagctgtgtgagcattttctcttggtaagttctcattttcccttaatcattagaagacacggcacagtaaacgcgagctcatgagcagaaggctacatagttgtatacatgtgtgtgctttttttcacaccagagtttgatgaagcaaaatacccaaaggtcttttaaaagtaaagaggttttaagagtaaagaaatttaagaaaaaaaacaaaaagaagtaaagatggttaggtctcctgtttgctctccttttgcaatcagtctgtctcgctctgtgagctgactatgctgcagacttttcttttacctccagcctttcgcttgtagggtttgaggctaggggcggggagagagagttcagcccagcaggcagcaaaccctatgtgcaaacacgatgtgtgaacattgtgtgcttgggggaactgggattatttagctgagaagacacgaaatctcctccccttatttgggctttgggatctgagttcattggaagctgaaatgtaagagaaagaagaaagggggaaggagtgggacaaaggagaggggagtgggagatcagactgggcacattctccctcacagagggaagcgatttctttgttaggaaagcagttctgttttgagacccggatgccttgtttatgctcaggtgtgaagctggcttaggaattgagtttagcttgcctctgtgcttttatgaatgtgttggctgctcagcaaatttagtacagaaacattttatgtaagcagaagagaaaattatattttaaaagttgttttaacagaaggtcagagtatgggattgagaattaaaaaaaaaaaaaaaggaaattctgtgttctaggctttaagacagagagtcacacacactaatttagaccagctggggtgatctcacagggactccatgggccagagctgccgttcctccatggagagcacatttcccctagaaatgtgtactcaggtgcagctgaagggtctgaggtcatggctgcatccctggccagggtcctcccaggctgcagcctctgcttcctctgttgtccattggtctagagccatccttcgagaccagagccagaccatgagcgagccaaatgtgcatgaggacagcatgaattatccttggagaccagggggccctgttcatccagacattgtctttctttttaaagaagctaaatgcctctttttccctctttcttcttgcatgacctctgggagcttgggcagtaaatttctctgggacaggttgtgaggaacctcttcaaagaagcagagatgagacaaactgtttatcagttcactctctacccctgctgccctcccaatgctcaggtgggccctagattcatccagaagccagggtgttgtgcttcatgaaagggccaatacccctaatgtgggacctatacaataaaaggcaagggcagaaagagagaagaaataaaaggaaaaggggtgtcttgaggggagtacatgatagactcctgtgcctggaagggactaggaaacttatactgactggttcactgcatttgctcaaagagtattcatcttcttgagtttgtcaggtgactgtaagagctgggttcaagttctttggttcactattatatgcctagctgagcacaatgcctgggacttagagggaaggtctgggacttcaccaggattcccaaaatagaacaaagcatttgttatttgcaaaaggttcacaaagcctgaaaaaatatgtgttgtctctggtggaaaagtaagggaatatagcttaagtacctactacgctctgactctttggatttcacctgcagggcttttagaggtggataatcagggccatctttttcgggggtggggggcgaaaacaaccactgaagaggtgaagaatcttgtagttctaaaaggatcagagagaggcttaaaacaacttttgcaaactgaaatatgccagccagcttaaattagcaaagctggaactgtggcagaatgaatagcccgtgtctgagtgagagggaaggaaagggtctactgtgtgataggcaatggttccaaagtaggagtaggggcctagtgtggtcatgccttctgatatgtcaagaaaaggcaaaaagttgatatttatttgaaatattctgattttataggttggtgatcaattaaaaaactaacaaatacagttaacaaacatgttttaatgcttgcagacaggatccttcctgccgatgtttggcctttgtctttaatggaatttgggctgccccataacagcccaccctaagcaaaacttcttttctgcctttcttttatattgttttttatggaatgtagagggatttttctggaatttttggaacggggaaattagtgacaatggtggtgctgagattcctgtttatttcttggcttttccccaattccctgagggaactcatttcttttggatcttccataacatcggtcacatagttgggcacagagagggcccacacttggtacatgttgatgaaataaatgtgtcttcctctaagtctagctgaatggagaactgaaatattagggtgggtgaaagttacctaaattgtttgctgccaaattttgctgtgttctttgtctccagttctgtgaggtgtgtgttagatacctattgctgtataacaaatcacccccaaaacatatctgtttaaagtaacaaacatttattatagcacacatctgtaggtcaggaattcaagtgcctcttagctggtactctcatggggctgtaaaggaaatatcagttggtgctgcagtctcatctgaaggatccactccaaattcattcatgtggttattggcaggattcggttcctcaccctatattggactgagggcctcagttcttcactgattgtttgaccggagctctccttccattctttgccatagggcctctccatagggcagccccgaacatgggatctggtttccattagagcaaacaagcaaaggaaagcagtgcaagacagaagctgcaatgttttggtaacctaatctcaggagtgaccctcctgtcactgcttctatgctgtattctagtcattaaaggagaccatctcacactcaaggggagagactacacaagccatagtgctgccatggcaacacaaccattaggatgatgccttgagccttccttcttcatatttcttagacttcctttctgacctagaggagccagaaggacttgggacttgtcataccgtccaatcccaagaaatgacaggaatgaaggaatttttaaaaattcaactcatttcaacacacacacaaaaaatgagagaagtgagtcaatttgagaactgaaattctggtagacttttggggagacactagaagggccatgttcatgagtaggaaccatagattctgagtcaattgggcatgaaagtttttctcctggtgtcccgatcttggtcgtatagtgaccaagcatccaagaaggggataacacaatgttcttacagtgttttctctgttgtattaataactcaggcatcagccagtggaatgggcaaagctaagtgaaggtttgaatgccagacagattctccattggctaaacccaatagacatgaataaggaataaggccaggaatcagcatccagatatattaatttatgaatctgaggttattccaaaaagcaagagaacaggagcaccattggctgagacggacaccaataccaagctctagagagtaatttcaggcagaaaggaaaccttaattaccagaggttcaagggataggcacacagtaagtccaaataggcagaaagaaggcatcagggataatctggcagagactgcattcaaggtcaagggcagggcatcccagctgctgttgagactgggctccaaggaagtctccctggagagactgaaaaacaatcctgggccttaatcactgaggactgaaacacaacataggagaaaactagggcttaagagaggcttggtcaagaaataggcaagaacctgattattggggaacaagagatccacagtcaggctggaccagtagcaagacagcaagagtagacaaaaagttgaccactgccagaaaccaagattggtctctgagtctggcatggggctgaaactacttgttgagatcaggctgtctcacttgccaggggtagagtcatgctggaagtggagccaggacagacattgaaccctggctaccctgtgatcaattgcactccataccctttgatacctcatgggaaaatcagacaagtgctgttctgaaaggcaattaggtttcatgatttcctcccccatccggacatcagcatgaaattgggggcttggcttccaagcccatgttcttcctttttgtgaagggcagggtgagacaaaggacaccacaggccagagcaaccctctgcttccatacaggccctagaagagaagtgaggacgcgggtgacccacacggccttctgtgcctcttcaacagctactctcccagcctcccatttctgccaagccacattctccacaggactcaaacatttagctttactttagacttttccaggaatcttcccaaatctcctttcctcatcccccctcacctcttcctcaccacccacagccccctcctccacccccatgtacaacatccggcttttactcatgcttgtttctaattatgcatactaatttattttctttccttttttcctgacctgcctatattttcctactcattctttggcctccactaccgaaaaatggaatactcctcctgtacagggtcagggtttttccttccccagtgcctaaagctggattcagtccacagtgacttctctaataacagagtgtaagggactccttggttgaaaagaaggaaaaaccccaacttgcctttcagggtctcttcaatgagcttgatttgtgttaaatttaggggctcaggaatttctgccgatttgcagcacttactactcctacccttcccaacatttgtgctttatcgaaacaactaaattaggagcagcttgagggtaggggtcagagcacagacattttctgtattgcctgtagaaactagcacagtacaggcaccccctaagtgcaaaagaagtccttgctgacccagatggaattttcctgaagtgagtgctttgtgtctatctttagaactagactttcctgataaaatagtcaggagatttggctgaattttccagtattcaactggctatgttggggtcatgtagatacaacctttaagaacataatgacctttaaaatatctattcctgaagtcaggtttggtccctagtctgtgggcagatgactgttttcatggtatgctttgttcgtgtccttcctgggtgggggaggatttggggggacagtttgactctagttatctacaaagttgagttatgagaccagcccttttcaactttacctcttgcaaattgatcctgtaataaattgacatgacgaaaacaccaaagcaaccagagaaatctcacaaaggcaagaacaccatcacacaagtcctcaaaatctttcccaagatccactaggggttaatgccactgatctgatactggggcacagcctaggaggttttgctgtctctgaacgcacatgtctccagcttacagctcacaggccagctttgtttttgaacagattgtgttgggggaagggaggcttctgagacagagctgagagtccccagcaatgccatccctggcttctggcccatctgaatgccttactctttcatgtaagcaagacatgaggtcctgctgtttgagttggctttgccctgctccgtgatttcagttccttgtctctccagcgtgtgggctcatttccgtcctctgagcactattgtttttctgacttgaacagtcatccagaaaacctcagtaataaggcttttgtatcattatttactttgctaattatatatgtctttcccatccccagtgcttgaagacatcaaattgcagttgaagtaacggcagagggaaaagcaagggcaacaacaacaaaccacttcactcctctgggtggaaacatgcaattgtaccaagagggataaggaaaaaactgctcatgtttgcagaaagaagttggaataagggcgtgaaagcaggcgggctactttagcatctgatgccagggatatgagagagtagcccagtgtgttcattcaaaaagtaaggctctgaggccaggaccaggaaagatcctggagtccccagtcacaatggcatgaagcaggatgatgcagagaggctaagggggtgattcagtgggttctttacctataaaaatgatggaaaccccaacatcagctgggcaagtggccctgttcagagctcctggactcgccatgactaaaacactctgttttatttcgtagttgatatctattgttcttgcctcccagcaagcaatggaacagcacctttactttcctttgagaaactattcctttcccattctcagggcttgtgaggatcaagtgaggtcacataatccagatctggccaatcagcccatctcatgctactggctacactaattggtttagggatagtaaagtactcaaatttgtttactgaacatttagccctggaattttggtgatgggaggtgtagagggtaaactcttgatgatatcatttgtgtacctgggtccagccatgcctgaacttttcaattacttgacccagtaaatttccttattcatttaaatgttagagttgagttttgtctcttataactgaaagagtgacatgtgtttatattaagtgcaggtcactaatatagtctattggtttccccaggcttgttctccttttctcaaataggactgatcagatagtaagtgctccatacctgtaggatacataaaagagcaaatgaacaaggagcaggagatatggtggctgacggttggactataattttttcttgatttttctgactttattttggctgtgatctaggaagtgaaatccaggtccttctaaaccccaatttaactgtggctcctgagggagattttcagatctcaaatgatctgtaagtttaagggaggatgattggtccctgtacactctctgtgagagttcctttggaatactggtttcagaatgtacagccacacaggtaactggattagggacggccagtggttttttatgaatgctattgaagacactgaatggaagcaattctcaccagaactctttttgctgtcatctttcaggggctaagttgacactagaaccaatcaatgatccctgaactgggagaaatgggtggccttggctgtgatttttggttgtgctgcacatttcaagatgacaatattgttttaaattaacctgttagagtgcacctttcctaaagaactttaccttgatgtctgagtaagggcagtgcaggaggtcccccaacttaggggactgatcatgttccaaaggtcatttctcagtctggatgttttcatgagataatgaattccaagatattgtaaggttcaatattataaatggttcttagtttactaagataatgcattgcctctttgtgaagccttctctggttaccctaatcaaaagtaacttttctttcctcttatatgtcacacagtattaaatgagttgttttttaaaaacatcttaaaatcttatacttaagcaattatgtagattatcttggagtgttgagcttattgctctcattagatgttagtaaatggaaactgagtccacaattttcacactgttctagttctttttcattctgcttcattttctgttgtctcctgccaggtgtctgtgctaactcatcttgcctgaggacccctaccccatgtctcactggtctgcttggcttacttctgaagcccagcaccaccttccctcagtggctcttaacaataaaccctgacacagaagttctggctggtccacctattcctttgagtcaggtcacaggtttccctccagctaactgatggatggactgtccttggctcaggggtccactcttgttcaattagatacattttgggggcagggtcagaattcataaaatgcaggcatttgaagaacagggaatcaaggtagagagttcctagaagacagagcagggaggtaaaatgaaatattctagtaaagtattaatatttgtggcaggatatggtcttttttctttctttcttttcttttgagatggagtcttactctgtcacccaggctggaatgccctggcataatcttggctcactgcagcctccgcctcccgggttcaagcgattctcatgcctcagcctccctagtagctggtattacagatgtccgccactacaccctgctaatttttgtatttttagtagagacggggtttcaccatataggccaggctggtctcgaactcctgacctcaagtgatcccccgccttggatatggtcttaactagaaatgaagagatctgagttcaaattcagcatttgtcacatgtaccctctctggtcccaaggaaatcaagtaatatctccaatcttcagatctttcacatgaagaataaagagtattgatcaccatatctctacacctccttccagctctaagacctccaggactcacctagaaccaaattcatccccacacagtggcggcagggtcgtgttgtcatgatgatggcctctttcggtgaagagaggttgtcagacacctggagcaatgccagagacattatccttccctaggagagctactgataagaaaccactggcagctcctcattcacttctccatcattcactcttttctcctttctttttccttagaggcataaacaaggtgtagatcatcctgttatccataatgaaggaagattttaggatattccagcttagatccaggtgtccccagcttctatactcactctgaagaagtgtactttatctactctttttacaccatttatcccattttggccatttctgaaaggaagatcctagaagccaggcctcttggaagaagttggagctacagcattgataggaaaacctgacacctagaaaggcaagtcagttcttcccatgcagtggctttggggactgtaatttaacaaagctcttttttacttgtgtaaagtgatctccattccacttttttcttgcactgattctaattaagtggaagcaattgttatcatcatttaggattatagttaaaatacccctcaaattgtttccatttggaatgggaccttgtcccattctgctcttgctgaaggaagtcttcatggaatcacaggactcaacaaacatttctcattaaaaattctctcctttaggcaacagtggcatagaaatccttatgttctactgtctgacccgtatgagaacagcgcacttctcaatttataaaagaggcattttccctcctcctgggaaaaatatttctcttggtcacttgagtctagcatctaagtcatataatgctattatagccttgttttgcttttgatttaggtcatatcttctttttctgatctttcttaaagtgaagggaccatttgctatgctcacaaaaattctcaaggctggaactacagcatgctctggtttctaagccatacacatccaggtagaagggtcaatttaattgccaatgatctcagccttttctaacactgttccttaagtttctttggcactgcatggcaagcaactgagtcatgcatgaaaatttctcttctagacaacccatacctacagagggaggatttggtttactgcagatgctgagtgcttctgatggcacacattccttcttattaatatccccctggcccataatggtttgtgtggacaacagctacttccttcaagggtcctttgcttggtaaatatatgcctccaggagctttctgggtagaagcagtcacaaatcacaggggtatctatgaatggcacggcccatggaagggctcacactgtcctgatttttctaccataagagctaaaataagggaataggcttccaggaaataagtcagatgggttgtctgactaagcacaggggcagggattggaggtgagatgacagagaacagtcagcattgtctccgcttccgctccaccctgcagttctgagctgaacagataatttatctgtggcacaacaaagtatatgacttggaagaatgttaaagatcaagtccaccttctccttgtattatatggctctgagagccagagaggggaggtgaccttcccagaatgtgaatgtggtagagcctagcttacaattcatgtccaacttaggtgtgaagttctatgagactatgctgccctctgcctcacaatgctttctcccatgggagaggaggctcagaggtacaaagcagctgcagaaagactctctcaagctattgactcttggatgacaacaattaatgttcaacttggctttacccccaacatgcactgcacagatgcctgcaggtctacaatgccgagaccaagtttggggggcatttccgcagctctgtctattgtcctcctttcctcttctgtggtcattcttcccaagggataggcctagtccagggtggagaaaaacaattcttggtggtaggggcattggaattgtatgggcagggttctaggccaactttggcctttcacaattctgctaagcattgaacagaattttcaccctaatttcaccttcccttccatcccttccctaattttgctagtgtgtatggagttgatttgatgagacctcgtctttttttttttctcccttgtaacgtgggccctatcttcaagcctatcttcatagcctttcttttccagaccttgccaaaaagcagtagaagagaggttacaaaatataagaggaaaccttaaaacaaacagggtagaactggagggaatcttaaataatgcctaacccaactccatattccactgataggaaactgagatcctgagacaccatccaaattctgatgtaatcagcagaacttaatatgccactttgagcacagaaccaagtactttagaggtaagagaggctatttgtttccagcacaggctgaaacccacatgagaaagttggactttaagtgaccatagtcacattctgataaattctaaaatgtctctctcattttctctgacatcatattgtcatttttcataccacaaatgtgttacagtgaaaaccaggtataggtcaatagatagtaccaagaaaaagaaaagttatcattctttaatgctctttagtttgaaaactttatttatttctttattttgatggaggagtagagtgggtcaagctaagagttgccttataaacctgttgttctcagcaaatgagtcagctgagataaaaacattagtttccttggcgtgtcagcatctctcccaaaggagggtattagttcatgggttggcattgctatcacaaagaaggctctggcagcacctgattggcttggccaagcttttcccagttggcgtctgggcatcttcctagcagtgggtcactcacccagcagtgatttggcctcctgttgacataaatatgcctaattcaaagcagaaagtcaatttgttattttgcctaaagaaatgtaactaccaagcaggctagatttctgtcttggcataaccaggaaatctttaccaataatttatgatgtggtcacccttgttgtaggcattatggaagatggaaagatatataaggaatggcgtcttgcaaaaagaactggacttttttttttttttttttttttgtagcaatgtggagagtaaaagcccaggggtcaaatggtaattatagaaggagattcatttaaagaatgtcataaggaaatagaaaataaaggaaacattgcaggaaaccatagggggctagctgattcatggaaaatgtgaggttatggctgggtctaagaataagtaaatcattggtgactatggttgtagaggttaggacagagaaagctggagcaacaagcaatcatctgccagataagaccccgggctgggtgaatgcaaggagggagcggacaattttgtcttccaactctgtggagctggagccttatcttaggagtccagtgggcctggaggcctgatcaccaaccagtgtaatgttagaataagacttccctaagcaaatcaatgtgatgattgatacaaacagccatcactggggcctcctctatttccaatctcttcctcagtttcccagttctttcagaacctcactgggaagttcagggatatatgcctgcaggctggcctggcgcagatggatgatgaagggtcctgccttgagtgtagggtagagtgattctgagtgctttccaacagactgtaaattaaagacactccaaaaagcccctggtggggatagatggggagaaggggtatcactaagtcttcatcttttcttctagggccagggtcagtggttcaaataaacatggtgccccacagagatggcctgtggccaagtagctctcagtgggttgactcaaagtcacctggacaggcctggcggaaacgcaggatggcccagccctttcctaataggttttccagagttgatccatcattctgcccagaaaaggggagaataaaaaatagatcacgagcccctatgactgtgacttgtgcttccttggaaatgcaaacaggtgaaggagaaaatgacacagctagggtggtagggggcactggaagtcaacacatttctgggttcaaatgagaagggtgaggtgctggcatggaggaaactggcactggccgtctccaggcaggactttctgggcactgggcaaggccatggctttgggagctgccccaagctgctggtcgtgccagtgcttcattcaccctgtgaaatgtattaattagtgagtggttttggctcagagagagttgctgtggcttcagatggatatagcagaaaaaaggcaaatccatctacaggcgcctctgagaaatctcagctcttgtgagcctgagaagagcttccatagagagaaggcgagaccccagcaaagttggacggagaaaattgagacacccacaccaattagtatgagtctatcaatccagtgccaccgaaacacaagtctaagcctaacagaatctgtgctgcgaataagtaagattgttggaaacagaacaaaaaaaaactttgcaaatggtgcttctttaagcttctgcagttctcttctctcactcccttttcaaaaatggctgtaaaaaaaatttttggtctggtttagccagagcgcgtcagagctggtagatgccttcaaaactgtgatctccctttaattgtcagagggtaggctgaggtccagggaggtaaacagacttttcctaagctctgtagctggctactggcaggattgagggaaggtggaacaagcctcttcattcccaattcaggtttctttcttccatcccacatctcatgcaataaacatttccaaatctacctgcccagcggggctaagagcagagtggtctccagaataagtggaatagcatctcagggactgctggtctgaacacgtactccgaacatcacagctgctgccaagcgggcagacgccttagccccagcccctctccctgggctttcctgggtcttgtcgacacttctcacatcactctgccttaaatttttctccccctagaaagagataacttgcctttcattcatgcctacctaactcagttcaattcaagtacataaatcctgctgacaaccctggcagaagctttatgagtagggagtaaagattgtgagaaaggcagggaattaatttataaggaaggaacaatgggagaggagagaaacaaagagaaggatgcaaagcaattaatgttaattgatgcttaatatatgccaggcccagtcatcctagttatttcatttaatctttaaaataatccttgtgttgtcatccatgcgttttcagttgcagttccatgatttaagaaaagcacacagccagaaagtggcagagaaaaaatcatatcctgagtgacagctttaccggcccagtgctttccattctaacaaatagtagaaagagggaaagagagaagaatcccttggtgataggaataaggtagagagaggctgtgaaagagagaaacagggctttgggggatcctgggttctgatgctgacctctgtgtcatttctgtatccattgtatgaatgtttatggaaagtcagctacgaagcaaattgttctaggcaggggggtttcagcagagcttgggagagcagatggtgggaggagtgcatccataggatagcctgggctgtgggaataagagaacagcaagtcagggtgagtgctgcaaagacaatataagagagcgacaggcctcagagaacctgggaaaaggaatgaatggggtcaccttaggaagttcattaggaggggcctctctgaagaggtgacctttgagtcgagacctgaggcactggagttacaggaaatgtgatgttgcacgtggagaatttcttggaaatggagcagcaatgtagtctgaccctattgttgggcccattaacttgggcaatgtctcaccaaggtggaggatggtgacaggtcctcctgggatatgggccatggcaggacgaggggacaatgtctgtaaaggatttaaaaagagtcatcaattctaacgatgtcaatgataaaatacttctccatatgcagggggccatttccatgacccagctcataaggagtggagagagtgctatcttcctctagaggcagctccaggaagcaggagctggtaacctgggggctgctctgaaaccttcaaaagggcaaatcatgtaggagtctctggccactttcttttctgtaagagaagattcactcaaggtcaggggaccctaaggaccccagggaatcacccaaaaactccagccagcccctagaggaagacaatccacagagctggccccatcccttcggcctgtggctaaggcccatctgcccatggttccttcttgaagtaattgctgtaagaatttgtcattcacaaaaggaagtctctattgtctcttttcggatgtcatgtaaccaatgcgtgttaaaggtcagttgcatcagttctcttaaatggcaattaagctattgtctgagcaaaccagaggcatccggagtgcaagcttcgctcaaacctagggacctgggaaaggctgggaacagaggagcaagtaggacaccacaggggagggccgtggcttcaggaaacgtggggaacaccctgaactgtaaaagcagatgcgtcagcagacaaagggggtcatttcactaaagcctcagtgaagcagagaaattccagactttgcccatcacctactggcaggctctccctccctctgtcccctccccaccctctcctcacccttgtctttctctgcttcattatctcacgctctctctttctctcccgcagtttctctgtctctcagtctcgatttctcacatcctgtttctctttcttctttactctttctccctctccttcacctctcctggcttcactccactccccccacccactctgttccctctcacattctcatttggggatggggggcggctgacaaaccggattgggggggtctgacaaactggatttaaatttcagctccgcagcgttctcagctcttggtcttggaaatgttccctgagcttcggtttttctcatctgaaaatgaaaaaaataatacctagctccaagtattgagcttgttgtgaggaatcaaacagataacgtttctaaaattccaaacacagtgcctggtccacggcagatgcttgactggtgacagcttcctttcccagtgaggcccagggaagtaggcacagtgcttggcaccctgcaagcatgcgacacagcttcacaaaatacccacagggagccaggagcaccgcttgaattttatggatgacaaaattgtggttcaaggaaggtaaaagtaccctggtaggacacgtaatttttaaaaagtctatagctattgatattactatatattaattcccattctgtccctccggtttctcccctgaacactaatttccaaatccccaacactctatggtttaaatgataaacgattcacccaaatactgtataattttttttttccttttgggtcagggccatgctctgtcacccaggctagagtgcagtggtacaatcgcagctaactgcagcctcaacctcccaggcttaagtgatcctcccacttcacagcctcccaagtagttgggactacaggcatgcacaaccacaactagctaatttttaaaatttttggcaaagacagatctcactacgttgccctggctggtctcgaactcatggactcaaatgatcctttctcctcagcctcccaaagtgcttggattacagatgtgagccaccgcacatggccatgtacaattcttgaagaagctgaaactcatctttctttctcggtataagaaaactcaggatatgtttggacatcccctcccatctgccctcccagcctcctcttctccaggctgaaccagatcctgctgcatctttctcaaagactctcttacatcctcattatggtttctgccccagtcctccatgcttctctgaagtttagagtcaaaatgcccttttttgctttaggggtgagaccttctctagcaccgtgtgtctacaacacagagagggcaaacagagatagtgcttggtattttatttttaaaatttttaatcttgattatattttgcaatgtttataccattgcacaacacacggaagcattagtcacaatttatatagctgatttcatggagctgaattctatggtgtttatagccgttgagtgtcttctcccagaaattggaaagatgccccatttgctgtaacacacacgacctccaccccattcttttcctttgtaacccaaaccacaatgtgtaattgtgtgtttattttgtgtttaatgtttatctcttcgcccaggctctaaggctctatgagggcagggatcacatctgtttagttcacccagatttcattatatatccagcattctacactctgtaactgcttagtctcaactctgaccacacattaagtcatcaagggagttaacagaaaaaaaaaaaaaacaataaacaagcaaaatgtttactaatgttccacaagctcttttgaatcaactggtccctgtggcacctggggatccgtagtcatgaaaatcctgccagggattcttgatccgggatctggtgatcctcggcaggacaactcaccattctgttcggttgcccgcttgcctcctcccgctggctccagcttctgtgcctacaatgaaggctgtgcccattccctcacgtgcccgccgtggcctgacaccttcctgggcccaccctaacacattcctccgttgcttttaacgactgtttacagctcgctacctctaggacgtggcttctgatgaaaactgtccagacgctctgcctccggtgtaattcagagcccagctctcctccttaaaattcttttacgctattttccacaagcatgtgcttctgttggcagtgccataagtgtgattgatctttaccataagttccttgaactttgctgtgacctggatgctaaggtggcgggagctgcctccatccccacaatttgactggtacaacccgaaagtaggatgatagtctgttctggtcaaacattaacgtggggcagggacaatggagagagggtttggtcatctggcatgctgcagactgccagaggagctggaaaggctgagtatgggaaaggggagggatttcatggcctcttcgggctttgagtatttgaagaaactgcctatggaagaccagacctgtgccctgcctcccaagaaacacacagagcttagacggtggaagctatctggggcaaattcagctccctgtgaggaaggtcttctagttgttaggggtgtcttggtaggtcagggagggttttccagcatcagtcattttcataccaatctctcggtttttgccatattcctgtaccacccgtgcttttatttacctaatcttttttatttaccgaatttttaacctaatatttcctttaaaacaactttaaattgacacactttgtttcattcctgtttttaaaaagtacctatggcaaaagatatgaatagacacttctcaaaagaagacatgcaagccgccaacaaacatatggaaaaatgttcatcatcactaattatcagaaaaatgcaaaccaaaatcacagtgagatatcatctcacaccagttagaatggccattattaaaaagtcaaaaccaacagatgttggtgaggctgcagagaaaagagaacacttatatattcttgagaatttaaattagttcagccactgtggaaagaagtttggagatttctcaaagaacttaaacagaactaccatttgacccagcaatcccgtgactgggtatgtacctaaaagaaaacaaattgttcaaccaaaaagacatatgcactcacatgttcatcatagcactattcacaatagcacggttcatggaatcaacgtaggtgcccaccaacaatagattggataaagaaaatgtggtacatatataccatggaatactatgcagccatgaaaaagaaggaaatcacgtcctgtacagcaacatggatgcagagggaggccattatccaaagcaaattaacacaggaacagaaaaccacataccacatgttctcacttataagtgggaactaaacatcaggtactcgtggacataaagatggcaacaataggccaggcacggtgctgatgcctgtaatcccagcacttggggaggctgaggcgggtggatcatatgaggtcaggagtttgacaccagcctagccaacatggtgaaagcccatctctactaaaaatataaaaattagctgggtgtggtggcacacacctgaaatcccagctactcaggaggctgaggtgggagaatttcttgaacctgggaggcagagattgcagtaagccaagaaggtgccactgcattccagcctgggcaacagagtgagactctgtctccaaaaaaaaaaaaaaaaaaaaaaaaagatagcaacaatagacactggtaacatacccaggtaacaacatacccaatatatgcatgatatacccacacaacatacccaggtaacaaacctgcatttgtactgcctggtgggtgtggtttgtgaaccggtatgtgtggtctgtggtatgtatggatgtgtgtagtttgtgaactggtgtgtgtggcatatggtgtgtatgtgggtaactggcataactggggtatggtaggtatgggtatgtgtggtatgtgaactggtaagtgatatgtatgagtgcgtatggtgtgtgttgcgggtggtgtgtgaactggtgtgtggtgtgtggtatgcatcggtgcatgtgatgtgtgaactggtgtgtgtggtatggtatgcatgggtgtgtgtagtgtgtgaactgatatgtgtggtttgtgaactggtatgaactggtgtggtgtgtgatgtgtggtatgtatgggtgtgtatggtttgtaaactggtgcatgtggtgtgaactggtgtgtggtatgcatggagatgtgtggtttgtgaactggtgtgaattggtgtgtggtgtgtggtatatatggatgtgtggtatgtgaacttgtatgtgtggtgtggtttggatgtggtgcatgtggtatgtgcagtatgtggtgagagtgtggcgtgtggtgtatgtggtgcatgtggtgtgtttggtatgtggtatgcgtatggtgtgtgtggtgtatggggcatatgtggtttgtgtgtggtgtggtgtttgtagagtgtggtgcgcatggtgtgtgtggtatgtggtgtgtgtgtggcatgtggtgtatggggtgtatgtggtttgtgtggggtgtgtgggatacgtggggtatatgtggtggggtgtgtggtgtctgtgatgtgtggtatgtatgggggtatgtggtgtgtgaattggtgtgtgtggtttgtaaactggtgcatgtggtgtgaattggtatgtggtgtgcatggaagtgtgtggtttttgaactggtgtgaactgctgtgtggtgtgtggtatatatggatgtgtggtatgtgaacttgtatgtgtggtgtggtttggatgtggtatatgtggtgtgtgcagtatatggtgtgtttgtggcgtgtggtgtatgtggtgcatgtgtgcggtatgtagtgtgtgtgtggcgtgtggtgtgtgtggtataggggtatatgtggtttgtgtggggtgtatggggtgcgtgaggtgtgtgtggtgtgatatgtggtgtctgtggtgtgtggtatgtatgggggtatgtggtgtgtgaattggtgtgtgcaccatggtatgtatgggtatgtgtggtctgtgaactggtaggggtgctgtggtggggagtggtgtgtgtggtgtgtggcatgtatggatgtgtgtggtgtgtgtcttgggagtgagggtgcttatcatgagcaggtgctgcagaagccctcagggaagatgacttcactgggagctgagatgacctcactgggcgccctccttcagcagatgcgaggacaggcccacaggcctaaagtcaattagaggcagatttccaagcagaatctacttctcctgacctctgattcagtgccttttcctgacaattttatccctgcgaccccagccttgagaatgcaaaaaactacccccaacttcagcctgaggtcatgaaaccaaccaggagtgcaggccgtctaggggaggtgcgagtgcccacctactgggcagccactctgagggcaaagtccacgggagacacagcttcagccaccaccggggctgcgacctgtcctcaggatcctcctcagccccctccaggagtcctcagctttctgcttttagccccaaacccagattcactttcccaggccattcaccgtctgaggcagcccagtccgctttgtccttcctccttcccgaaggctctcagcccctgggcagtatcagaatttcactttgatctccttttccctgggagccagaatagcttagtctcttcaaaaggagagttagaaagtgtgccgttcatctaataaaaattaactcctgagagttatccaatcccaatacaggtcatttcgctggaggctaccccgactaggaaacacattttaggacatcagcagaggtaaagatgagagcaatttgctcaggcttttacttggggttaaggatcactaggccaccaggaccagggatgccagacaacctcaggtttctttccaagctttctccaaactaggcctcacttttatcattttcctacccctgggctctatgattttgtggtgttgcttttgtgttttcaaaaacactgcattcattcattagctctgcactttccctcatgcaatgcacacagaactgtaaagacaaatgataaagccctttcccgcttggatcttgtgttgttggccaggttcattcaaaaggccattgtttgatggtgattaagtgtgtttgttttgaatattcacctaaacaaaaatgatcaatttacacaattcttggaattcatgggtgtcttgggaaaacttgaagccacaaaaaatgttcaatacataaatgccaaggacctcctgctgtcaattaagaccctcatgatagtcttttgcttatttgctcgttaattcatgtaaaagtggtttgtgagcaaattaagcatcagttggcatgccggacatggacacatagatacagtttgtggtctggaggggaggtggaaatgagagtctcacaggctgccctcggcccagaacttctgtgtggaggttttgcataaaaatcagaatttctggattcttataaaaaccctaaagttctagccccattagggccactgttccactcacagccctgagtaagcctcccccatcagcccaggcatggaccctttgtcctgcctctgttgtcactctcccgactgtctcagacctaaccagctcctcttctgtttgtgacttgcccttatgggagcctgagggctcctacctactagatgaagctgatggggacattctcccaaatgagaaagcgatggaaagttgtgaccatcagaggaagatcagagatcaagccttgttgatgtaaaaggaattaaagttgagaaaccatttcctgcaaccagaattgtgaaagcatacaacgtagtaccaaaggaaaattaccgaagcctccttccctgaagaaccttcaggaaggggaggccagccgtctgacgtgatcacagtgatgtcttgccctgaaacaagaagcattcttcccaggtcctttccgccggaggattcttattctgtgatcaatgctgagctatgtggatgagtgtttgcgtggatttcttttaaaactatattgctaaataatcttgccccctggccccatgcacgttcttttttttttttttttttgcacccagataagaaaaagacttgaatgtatttttttctataaaatgcagtcaatattgttctgtgagggggaaagaagtagtggaggatgaagatggaacaggggatgaggtggtgaccagcatgccctcatggatagacactgagcagggagaggcagggatatgcatggagtaagccctctctctctctaggacaaagcaaattatgtcttcatgccagtggactctgcatattcattgaaaaaaataagatttaattaatttagggaagcaatatgggctgaccagacagccaagttcttcaagataggacagtaaaacattttatgtgagagctggccaatgtctggatggaagagggagggccagggagactggagagatattgagggggtgaggaggtttgaggaatccacagtggggtgagcttcatgaccagtggaatgaaataagggctattctggatggacgaccaatgtcagtgcaaggccctcaatctcttgggataacactggagttggaatacatatgaaaaaaaaaatccacaagtataaaagaagtaagcagaggccgggcatggtgactcacacctgtaatcccagcactttgggaggccaaggcgggcagattgcctgaggtcaggagttcgtgaccagtctggccaacatggtgaaaccccgtctctactaaaattagccaggtatggtggcgtgcacctgtaatcccagctactcaggaggctgaagcaggagaattgcttgacccagggaggtcgaggttgcagtgagccgagatcgcaccactgcactctagcctgggcgacagagtgagactccatctcaaaaaaagactggaatagagtacatttgtcataggccaactgcctccatgctcttgtctcacagagatgtttataacaagcttgagtagctacaactggatactttggtctatatatgctttcctcgtgattctttttttcctccttcttagcttaaacaaatgcacacagagggaagtggataaactattagcagagtaagcacttgccaagaataccgggggcaccaaaaacacataaaaacaggcttggttgggagcccgatgtgggcagatcacaaggtcaagagatcgagaccatcttggccaacatgatgaaaccctgtctctaggaaaaatacaaaaattagctgggtatggtgacgcacgcctgtagtcccagctactcggaaggttgaggcaggagaatcactgaaacccaggagctgcaggttgcagtgagccgagattgtgccactgcactccagcctggcgacagagtgagactctgtctcaaaaaaaaaaaaaaaaaaaaaaagcgaggggatcttggggaacctaggaagaagttacttaaatttcagcacatgctatatagttttcaggattagtccctctctaggtattcatctggggctggcgcctccttggctggcaggggctggtgatcgggactccccagcgagacagattgcacccagagcagtgaggccccgagcgcttcctggagcctggggaagatggcattcatttttaaagtccatttattgtgcagttgttgaaagtgggacacagaaatttggactgatatgcctaggccacatgactactgattgggccagggtaaatgcagtctttttgttgatggagaaggtcttttctgggcccagcacaaagcaatggatcttcagtcttaagaaatgcattgaaaaccatccttgtgcagtttggggtccctggtaggccaagagctggaaaggcccttagactctgagtgctgtagccccccttctaggtgatggtcaaaacttgaaaacggctctagactctggacagaaatgcatgagaattgagtgttcaaagcttggtgctttgacttcttttaactttctgtagactgaaatatgaataagaggaaggcatgaggcccctctcagtttatggagccatagctccaaacctcccactttccaacccagcaaccctgtgtgtcctaggaattgtagcgggtgtcccagaatgcaaaaatcactctgctagaactggaaaaggagacgcctgccttttctagcacgatctttagtaaatagattcaggaaatgtgggtcctggtcagagctggtgccacaagctgagtgccttcaggcagcagacttagcctcttcaagtccaatctcatcagtagtatcagggggct

SEQ ID NO: 6 - DNA, 99791 bp, Homo sapiens
ttcagtgagtcgacaccgcgccactgcactccagcctgggtgacaaagcaagactctatctcaaaaacaaacaaacaaacaaacaaaaaaaaaaacagagagagaattactaatattttcttctccaaccttaatgctcactccctgagctatgtggattatactagtgaaagtactgccttaacccactaatgtaaacagattccttttaagaagcctccctatcgagaatggtctgtaagagacaaccatgtcaacaagtaaagttccttctgccttccctcctggggtgggctaagatggacccttctcaccattcccaattacctgcaggaaggcagcaacagaatagagaaggagggtaacacagtttcctgtactttaaatacctccctgcctccgcaagtaaaacttgagccatgccaatgagggaaaaagtggccgcgatttcccaggtggcttagtaatacacagttttctgacacactgcaactccattctttcctaaaagttccttccaagcctgtaggcactgtaagacaaacagctaagcacttcctgagtgcctactatttgccaggctgtatgcagcaccaggaacacaaaaataaaagctatggccattcaggaacaagatttcaaaatgaaaaataggaggaaaatctaacatggatggaaaaaagccacacttttccatgcttcaagtttagtttccatttctaaaatgtctgtcctcatatgttagccagaaagccagtttccagcaagacaagcagaaataaatattatgcaagtcaaacctacaagaatgggtcttcaagaaagggaaataattttcggtcaaaatctaaaaatccatattcaacaaaaaaagcacagattgagaaactaattcaatagcaaaaaaggaaaattaccattgatccaggttgccatttttaataaaaagcagtaaggtcttaaagaaaatattagcattgaagttcaaccaaataaattactttgtcatcaaaatgaagaatgccagaaagttctctgaaatacaatttttaacaaaactgagaaagcagaggaagtattttttaatattacaaatttttgacaccaacataagaattccatgatatcctaaagagcaatgaacccaggtaatgcagtttctagaaattttaaaacactaccaaataatattgcttcttgcattaagtatagcattggtttgctagaataatcctaacaatgacaatgcaatgtactttaaattggggtcaaaaaacttttttcttaaagtaccagatagtcaagcttttaagctctatgggtcatccacagacacactgcaactatacaaactatatcacccctgttgcaactacacgaaaagcaggaaagcaaccacagaagacagacaatacataaacaaatgagtatgactgtgttccaataacattttattaataaaaacaggcaactggcccatgggtggtaatttgctgaaacttggtttaaattattattagtatctacctttttttaaaagatgatgttttttagggtctttccatcatgaaaattatgtttttatgaaagtgatttgggaagtattaaaatacagaataaagagcaagccttgggctatgaagtcaaataggactgggttccaggactgctgtgtgactttgggaaactgacctgccatctctaggtgttagtttcctcacttgtaaaattaagatgctgacagccctcacctcatagggtcactcactgcaaggaccacacaagtgaacatgcatacagcacttagcatagttatggataaccctgtacctaccttattatagcaattgctatcaactttcttctagcttatcattaatgatactataaaaacacccagcataacgccagcataaggatgtgaatgaatgagcaaatgaaaggagggagggcaatggcaaaattttaccttggtttccaagctaaagtataacctgagttttttaatgctttacaaaaaggaaaggcaatcagaaccataattaatatttaagctgtactgacaggttttccagggaaaattctatagaacttgttattctccaacttccaaaaataagcaataaattttttttaaagagctgttattttaagactttatttgaaccaaagaatgtgtttatggtaacaacatacatgttgtaaattaatcagtttcaatgttataaaataatacttccaagaataaatgtatcatataatctatgtacctcatgtcgataatttaaataaatacattaaataaatacatcattccgaagtccgctttatcattgtttgtaaaatccaattgtcatactctggtatttgttctccaaaagaatcatcactttcatattcactacagcatgagatgagatatttaataaataataggacatcataatccagggattaagtagtttcttttagcaaaatttaatatgagggtagcatatgccagccgttcctagttgagcaacaatttctgtgttgggagaggataacaaagccaagtattttcccataagaagttcacactctaaaaggccaggcgcggtggctcacacctataatcccagcactttgggtggctgaggcaggcggatcactaggtcaggagatcgagaccatcctggctaagacggtgaaaccccgtctctactaaaaatacaaaaaaattagccaggcgtggtggtgggcacctgtcgttccagctactcaggaggctgaggcaggagaatggcgtgaacccaggaggcggagcttgcagtgagccgagatcgtgtcactgcactccagcctaggtgatagagcgagactccgtctcaaaaaaaaaaaaaaaaaagaagttcacactctaaaatgaagagaaatgagaaatgtctatgtgcacacaacttcccacatacacacattagttagagaccacagtcatgcccaccctccagctcacgtgacatttcaaagcagagatgagacttaaaggatggaagaaatggcgtttgtttcatggatggggtaacggaggcttttaggggcttatgggatggacaagtaaaggatcaatgagacagaaaggcaaggcaaggcaaggcaagggactcgatctcaggaagtggtgagtggagatgaaagaagtccaatggaatggcagtcactggagatatatttaaagaagagggtctttttgtttgtttgtttgtttgtttgtttgagatagagtcttgctctgttgcccaggctggagtgcagtggcatgatcttggctcactgcaacctctgcctcccgggttcaagcgattctcctgcctcagccacccaagtagctgggatttcaggcccgcgccaccacgcccggctaatttttgtatttttagtagagacgggtttcaccatgttggccaggctggtcttgaactcctgacctcaaatgctctgctcatcttggccccacaaagtgctgggattacaggcgtgaaccaccgcacccggccaagaagagggtcttttaaagaagcaagataggaagatccgttaaagagagaactgcataggaaggaaaaggcctggctaggattccctgaaccttctcatttcactactctcttgttagtcatcaatgcagttaagaatcaacgcaatcaaatcgaatcacatagtgattaatttgtgccaccaatagctctaattttgttaacttctcattccaataaaaataatcatctggttaatccatttcttcagcgggattacttgtttgcttgtttctttagctataataaactctgaatgccagagtcaccaaactaagcattaaacaaactgattgacaattcggaagcataggaataattacagatgatgctaggcaatatcactagattgggttttcatgaagctcgtgactggattttgccacaaaaatcactaactgctttggcttccagagaatggcctcatggataagccccaagttccaaaaactacaaaatgtcaaaatcacattcaactaattaataattttatgtcacaataagaattaggaatctctagaaggctgggtacagtagctcatgtctgtgatcccagcactttgggagatcgaggcaggcagatcacttaagcccagcagttcaagaccagcctgggcaacatagggagaccccgtcctacaaaaaatataaaaatcagctgggcatggtggcacatgcctgtagtcccagctacccaggaggctgaggtgggaggattgattgagcccaagagatggcggctgtagtgagccatgatcacaccactgcactccagcctgggtgacagagcaagaccctgtctcaaaaaaagaaaagaatttctcggaaagtcttaatttttatctaaacctatctgaaaattcaatgatgaattttatgaacttagccctgaactgacattcctggtgaaaactgcaatcattgtaggacaagagccgtcgtgttaaatgctaaggctattttatacacatccaatattttgggagcactgcaattaaaagtcttctcacactaaaaccaatgcacaaagcacctctcaggtttaagcctcactaggctgatgcctgaaacacagcaaaatgctcatactctttatacacatgtccaaaggtctagctatcaaaagacagataaaaaggagcattttcccccaactgatggaagcaagtccaaggtgaagaaagaggatagaacgagaaggtaaaccatctctcaaaggtgagaaatcaacacagagtcaaccaatgtcttcatctcaacccaacccaccaagcaatgtatcagattggattggagacagattggagactctatgaaagaactggcatgcacaacccaagaacctcctggattcttggcccttcttttaaagaaggggggtttctagacaggaatagaacccacagagtcatggaagccagccccctaccataaattgttcataaccacaagcttattggtcaggtttataaagtgattcagactctttagaggctacaggaagggtgcagataagaggacacttatgctggggtggggaattattggagaggcctccaacaatgagatatcacctataattacattaaagatttaaaaacatgattgaatcacatgttaacaatctcctaattagagctctgactctccttagaatgtccaacatccactgcatctaagaagcactagtatttattatgtagtattgtttgtgtcaaattcccaaaaaaggttatcttgaaggttaatggatttaagtagctggaaaccctgattagagttgcccatagaaatctgaattctgctcaaacagttcaaagctgacaacaaaacagtgatggactctgaaagtgaagagacccatagagttcacatgtaatgttaaaatgttcggcacttcacttctcaactggggtacatgtacacctaagacctacatcttagaagtttccagagttacagaatgaactggatcgtgtctcagattaacaattgattcaaagaatggggatagaaagggagccatgacaaaataaaggagggggctgttttgtatcacagcaaacaaggagatcctgtgtaaaagaaattatcaaaattgttaatgccagagggtagtggctcatgcctgtaatcccaccattttggtaggccaaggcagaattgcttgaggtcagaagtccaagacaggactgagaaacacagtgagagtgagaccccatctccacacacacaaaaacatctttgcattaaaaaatgaaactagaggctttaaagaactgctgcaggctacgtgacttaaagaatataagttctctgccttctttgtcatcaggagtgcttctgtgattacgtataggaaggcaaaccttaaaggaggtaactgaaatctttacttcaacatactcaattgtatgcacacagcttggtaagcaaaataagtcacaaataagcaatattgcttcaaaaaccttttttaaaaaaagatacataatcaattattttcatattaatagagcaaaggaataccatgaataatccaagataccaggtaatccagaagtcagatactgagaagtgaaatacatcagataatttttacagtgtgtctatgatcttaattacattttactaagcctaatactagtttttctttcttttttagaatttctagattacattatttgaaagtaaataatggcttcaaatcatgttaggcaacagaattcccaggaaggagtgaaggtaaatcagatgactgaactatattacaatttaagctgtgatgtttgcacaactataacaatattataaatgagcttgaggtttcttactgaagatgtttaatatttgatccaaaatatcgagcagttttgactaagcactgtaagcaaagaaatacgctatcatagcaggacacctgtaagtttccattgtataagaaaatgtttcctttgtttaattggagagtcaattaaacatacaaatatctgaagacattattatgaaggctgatttggaggaaatgtataatgcagatacattctgggtgtaagtattaaattcatacaaccattctggaaaataattcaacgttgccaagtcaagagcattccctaaaacccatgagttcctctcccaagtaaagacatgagaacaattcttgcacatgtgcaacagcgtgcatacacaagggtgtttgcaggacccgcccatgatagcaaagcactggtaaaaaatcaaatgtccagagggagtatactcagaccatggaatactgtacagtggcaaaccaaaagaaagacacctgcatccacttagaagaatattacaaaactaatattgaagcggggaaaagcaagtcagaaaataaaatatataacatcattccacttataaagaaaccaggaatatgtacaactaagcaatatattattaggggataaagagatgtatggtaaaattttatataaagaaaaaacaagggaataataaatgtaaaattcaggaaagtgactatctctgggggcaggaagggagggagttgcaactgagaagcgcacagagagaacttcaaaattattgataactttcaacaaagttggatgttagacaaataggtgatcattttattactattatttaaacatgctcataatttaaattcacatgtatagctatatattatgtattatagatataaaacaattcagaggtataaaatgcaataaaaaagtgtacaagcccaaatctctagcatactgccaatcaaaagatgattgttattcataaagtatctcaaaaaggaacattagactatttccagaaatcctggttgctttattattcatttattatttattaccccaggaagataaattttaggaagtttctactttgagtgtctttcttctaagttgcaaatacaaataaatgcttatttggaattataactatactggaagctcatgtttcaaataaagctatttatatatagcttcatcattgtttgaatgcttatcattgcagcctcattacaaagaacatctaaaagatttttttccaacctaagaaaaaacagtgctataataaggtatcttacattcatgggtaaaaatactccttttagagaacataatataattagtttctattccaagtctgtcagtgtgcactgttaacctctgacaattatgcatgaaccttttaattagcatgttatctttggtgcaattaataacttgtacttcatgttataggcaaattaaagttttcccactaaaaccaaattcttcagcatagttctaaaagtgttttgccatttaaatgaaattttaacattcagacctcttgctcccaaaacacataagccattttttccaaagctagacagcttttctctatccacgttctttcaaatattttacctaagacagagaatctcatattaaattcaatttcacacttgccaatcacacacgcccactcacaattcctccagatgatgcaatgtggcctaatgtgtatgcttatgtatgtacatgtgcacacacaaacaggcataatgtttaaatacatataaacacattcattgctaggtattggttaaaacaaacaggcttacagcataccaacttattaaacataatgattgatattagtagtttcattaatgtaattaacctttagatttgaattatcaagttcaaagtctaagaccgagagcagtggttctcaggccggtacaataatggcaagtcatggtattcaagagatgagtctaaaaggcccctcagtcctgcccctaccagaaaccagagatatttgctgctggtaataatgaggagggggtttctaagtacaagatcagctctattctttcattccctggaaatctgccctcgcctctgatacctcaaaaaatatctgttgcatttccaagggaaagaacaatgatataagccttgctgttaaaaaaaaaaaaaaaaatgccactacttacaagataaaaggcaaggtgttttgtttttttcccctttctcttagactaggcatgaataatctcaaaatatttgtttgtcaataaagtgattaaaataaagtgatagacaagacataatgattagagaaatgaatcaaagtaatatttgggaccagggtcttacaaatctagagtaagtatgttgagcagaaatagatactaaaaatttacattagaacaaaataaaaatggatttagtaatgctggatttaaattggaaatagctgtttaaactcatgaccttaaagaaaatttgtcttggccaactgcggtggctcacgcctgtaatcccagcactttgggaggccaaggcaggcagatcacttgaggtcagaagtttgagaccagcctggccaacatggtgaaatcccatctctactaaaaatacaaaaattagctgggcatggtggtacatgcctgcagtcccagctgcttggggcgctgaggcaggagaaccgcttgaacctgggaggtggaggttgcagtgagccgagatgggaccactgcactccagcctgggaaacagacagagaaggaccctgtctcaaaaaaaaaaaaaaaaaaaaagaagaagaagaagaagaaaaagaaaatttgtcttctggctctctccctaaaatgaactaactggggaatcatttctaccttccagaagcatttgaatggaacagcttgatatcatgcagagcacctaaattttggatgccactatgctctatcaaaggaatcagagctcctcagagaatagctgactccatgtattgggtcatgagaaaatcaagatccactggaacttgtggttcccagaaaacaacagtgctttcaagaatgcctgggggtaatgctaaggggaccaaagaggacatttaaagggactcccgcaggtcatgctgtgaccatctgagcatcgacatgacaatatgaattttaattaattagaacaagtgcatcccataaaagctgagagtccaggatactcaaagagaaacagtaaggctggctgggcacggtgactcatgcctgtaatcccagcactttgggaggccgagaagggtggatcacgaggtcaggagttagagaccagcctggccaacatggtgaaaccctgtctctactaaaaatacaaaaattagctggctgtggtggcaagcgcctgtaatcccagctactcgggaggctgaggcaggaaaattgcttaaacctgggaggcggaggttgcagtgggccaagattgtgccattgcactccagctctggacaacagagcaagactccatctcggggcaggagaagaaaaggtaaggcctactgaaaacacatttgtatcataataccaaagaagagtgactataagatgtcctaaaaatagatggtgaagtatttaaagattaggatactctctttctcctgtgaagtatgtgcttctttatgaggaaagaatcagcatttttctttttgtgtgtttgtaggacagaatgaatgcagagaattctaacccaaatagttccagaaaagtaggaatataccatgaaagatggcagcaatagtttgaagagtgagtcagaaaacaagtagtttgagaaccgttagcctatattctaatgacttgaaagtttagtgcatggagaaaaaatatttaatgtaaagtcatgtttgcctttataatttcaaagaatacttaaagcagtatgggaaatgcctaaatggacacattcagaactcctgagggaacgagcagaacacagagggcaagagaacttggaatcagaaattccacgttcaagcaccacgttgcctattcctcaactttaagaatgatcaatcctgccatatctgcctcccagggtaagtctgaggaactctccctgtggtttatgtttgttcaactgtatttgcaggtccatctttaagatagtttgcccatgaagatagcaaggtactttttatgaccctatctcaaaatcaggacatattctggtaaagacacataaatattggctcaaatggattttgttttacctgaatcaaagggattttgttttacctgaatcaaagggatgaaaatttgccccagaaatagttgaccaagtaccaggaggtttcaaaggaaaaactaggaagctgacagattgaatgttcaaaatgattccaacccaaaatgctctattacccagaacttgactccagagataatccaaccaacatgaacctctgttttctatttactcagagcatagttgctgctcaatacatccattcatgcaacaagtgccatattcatgatcccatgccaggcacgttctatgtgcttaagatgcagttagtgacaaaagaggccaaagtccatgcccttggtgactggggacaggcaaaggtggatggcaataaataagaatattagatgaaggaagtaaaataagactatgtcacttaaaatgacttggtgggtagcatacttttgaaaggcttctcagaagaggtgacatttttgagctgagacacaaatgacaaggagctggcattacaaagagctggggaaagaattccttgcagaagaaagaatcaatggaaaagctctgagttaggaatgtggctggcatgttattaaaaaaaaagaagaagaagaaaagaaggcttaaacaaagtgaatgccaagaagtgttaagatgtgagcttgcaggggaaggaaagagctagagaacacggtgtgagtaacgtaagcgaaaacaataaataatacctttcttcacatccaaaagctgatcagatttctttttccaatgccaaaatacatgtaagtacataatgtcacgttgcataccactgcggtggaacacaaggaaagaagagctataatgcaaacaaacagcaactgactctttggggagcttaagaaaaaagaagtccaggcaccaacacaaaaggctgaataaatcacaaccttactcaaagaacaatattaagggcataggctgtagaaaaggcaactggttcaatgcttctcatgccctctgagagctatcacaattaatggcatcatcttccaaacaacaggtaatacaatgttacttctgtgttatagaaacatttgggttataactggagatgtatggtatattcatcacaaagatgataatcatttcctctttgtgtattcaatggacagaaatctctaagactaggcctctgcttcccaaatattcatgcaactggaactgttacaagggtaaaggcatgaattaaaattatagaaatctagatgagatggaaattgaaaatacactaaaacctaaagtctcctatagttaacatgggataatatatgcatagttcctaacagcgtacctacaatcctataagggctgagtaaatgttagtttgctttccaaagtaaaagtgacctgatttttctgtgttgctcccccttgagatctaaagctaaacctctactgaataaaagcagagtgacttacaattgagataggccaatctaaaactgaaaagtctaaaatgtcaaacttttaaaataaggggatgttcactgcttatttgactcattaaaaataaaaatacctagtgattgtgaatttaaaactgtagacttatgatttgtgcattttatatatgttattcttcaattagaagttcaagaaataataatgggaggcctgtattaaaccatttatataaaagtttctcaaccataatggaaagatcaagatgtctagcataatttgaaagtatgtttctatttgacagaaaccttggcttggaaaagttaaactctaaatatttgtacagaaagaagtattctgggtttcattcattaaaagtcattgaccactgcagaaaagggagagcaagagatttacatatatttttcttgacgttttatatgcattaggcctggcaatgaacttgaggtaggtattactatctccttatttctaagggaatcagaatactgtaaacaaagacatggagctaaataatggagtcaaatcctggcttactaggctccacatccatgttgctccacttatcagacggccacccaaacatacaagtttaacaaattccaaattattggggcctaaatagacacttttttgtttcaatattgcccttcttcccttgtattctgaatatcatgtcaaccatgaatcataagccaatgcttacaaatttaacttcacattatgagttttgagcccaactgatgcctaaccactaaaccattcatataacacactgaaaagaagccatttaagaaatgagaaaaataggtattgttttctctgtatggctgtaaaagctgagataagaacattttggcaagttcaatagagccgaaaggcttgtgaaggctatgggaggatgagtatattggaagaagatcagtaggtcaagaatctaagaagagctatttatgtgggagacaggaattaccttgaaggaagagcagagccccagaagaagaaaaacagaatataacagctaactataaaaggacccagggccaggtgcggtggcttaggcctgtaatcccagcactttgggaggccaaggcgggtggatcacgagatcaggagatcaagaccatcctggttaacatggtgaaacctcgtctctaccaccaatataaaaaatcagccgggcatggtggcaggcacctgtagtcccagctacttgggaggttgaggcaggagaatggcatgaacccgggaggtggaggttgcagtgagccaagatgacagccactgcactccatcctggaccacagagcgagactccatctcaaaaaaaaaaaaaaaaaaaaaaaaaaaggacccaagagaatataagtcccatgaaaacagagctcgagtctgtcttgttttgtcacattacctatttgatactcaatatgtaatttaatttctgagtgaataaattaagcaaatgaataacttaaaagtatagctatgaatgttaaaaaaaaaaaaagaaaaagaaaaagaaagaaaaaagaaaaaattaagtggggagaggcatgcctatggccttgtccttggtcctgaataactgtttggccagatagtatggtttcctaaccctagctcatggtgttcaaattctactagtttttctttgtttgaactttccatctccatgaaactaaaaagtataatttttaagctgcagaacaaatcattccatgtcttattttctgaggttacatagctggctgcatgtactttcaacaattctatcatgctttgacacttagaaatattttccctaccagctttgggaagctgggcggtttgtgtttaccatcctctcctgatgttacgctttagtttaaaaataacagtcattatcactcaattattcagtgatcaagtgtaacaccaggtgcaaacaaaagttctgagaacactggaacatgctcttgttactgcaaacgatttaagagctttgaatttatgaagccagtgatcatgttactctaaattagaagcaggtaaaacagaagaatggcataagctaaattccttcttttcgtttctttacttcctttttccttttaaggatatctatagcaaactatattagctttctattttctccactatttgcactagcattaatcaacatccaaaaagtgtacaaattattttatctttataatcaatcttctgaaatgctaacttgaaaatataaacagcacaattacttttttctcttttctttcccatttgaaatgtgtgcttgtgtgtgtgtgtgtgtgtgtatctgaatctgtacatataaatcaaatacacacacatatacttgttttaaaaaaatcctcaaagaatcaatcacatattacagaactgaaggaatattggagttcatctggtccaaagtgttggtattgtaaaatcactttcaaatttcacagttatccaaatggtaatgaatattaatacctaagaaaatcaatcaaagattaacattaagacattagattttaaaaatcattgcatttaatattttttatccattttcaaatttcagaaactaggtctatattgtcctatagatctaacaagcactataaaagaaaaattacaaagatttcccaatatacagatagacacataaatgaaaacttttaattgcaagagaaagggaactggattcatcagttacatgcatttagaaaaatctgtctcattcatcagcagcaatgaaggagaaaagttggcaacagagccaagtatacttactttctaagtagccaataattggaggcatttaaaaatctttttcactaaaatatacattttttgctcaaactccagatttctgacatttccttattctactgataatgacttcatgtgagcaaaatggaatgaaagattgaaaaaaatcagatatatgctacccataaaggtttatcaatatgatatatctaaacatttatgtacttattggtttaacttagttgatataaatgaatatgcatatcatacacatcatgtgaacagaagagcagatcagtggctgggggtaggggattaactgcaaaggggtacaaggaaacttgtggtgatggaaatgttctatatttattatgtggtggttgttcacaactgtacatgtttgtcaaaacttaccaatagcacatttaaaattggcagattttattgtatgtaacatatctgactccccgaccaaaaaaaccaaaactctgttataccatgacacctaaaaataattcctacagagacttacctagaacatcatggacactcaaatattaaccatgacccaagaaaacactataaagtatgcataaacagtgattctcctataaaatctacacacatacacaatatgttaagaaatgagtacaaaaattaggggagctaccataatacatggcagaaacagatttcctgccccctgccataactatcatttaccaacattcaatcgaatttaagaattgggggaatttcagagagtatcctaattcacttcctaattcacttcctctagataccaccactcaaagattcccgtggttcccagagccttcacttgacattcatacaactttgaccaggaaagaagggtgcgccacctccacctgtgcctctgcctctatactagaaaggattaggcagagaaagagattcaaatactggaaggaaaagaaggtattgggaagccaggatggtttgttggggcaaagtattctcttgtcatacagatggctctcttcttggtggtgccccagcccaccctcaagggtgtctataagtaggagaagagaaaagcactaatgatgattataacataattaaataatttatgaaggataaaatatcagagtcatgagagctctgaaagaataggtagactacatgcatcttacctacacattgagtattaaaaatcctagctgccctctgcagtgatgccccacccagaagaccaatccattggaactattgcaatctgatcagattaaacacaccaaggtcactctgaaagtatggaggaaaacaccaaattaagaacaaaataatacttttaaaatataaaccagtttaaaagtatcaaaaacgtttctatttagtccatctctgatctctctcccaaacctctgaccgaaagagacacagatttttgcttctaaaaacaaagtatattttaccaacaggataatataagtgctttcaattttatctaataaaaagtagggcaaagtgaaaaacaggtgcctctacagatggagtatactaaatacatgtaacacttctgataatgttgaaccaccccctccccttttggaatttgaaacatcccgcataagatctgcatttttcaaatattgctaaatatttagagtctaaaaagatgagattcattattttgcacagtaacacatctcctaataatatttcaggccagttttgaagaactgaaattgatatcttttccgtatatacttaacctcctacagatttatttgattctcaataattactattctctggtaaaaggcatatgttattacagtacactgcactcccacaagctgcccattcagtgtattttaataaatctctgccagagctgtataaatttgatcctgtttaaatttaataaggatcagaatatatcaaagcacaaatacccatgggagaaatagatattttgcatagttattgaatggcctattcagggatacaatatgtaagacccagaccatcaaccttgaattaaaaagagagattaatgcgttcaaagccggctacttgttaaataacatatgaaattagtatttaataaaacgtcttctctatattgttgctaatggtatccatcctagtacagctttaatatcaatattctcatacagaattctaatatttacaagaattgtcagcaatgtatttcttaaatatctacattatcttgataaaatgcaacactgtatggaacagagatgaagaatacctcatagtaatttggaattgttgagtttaatgttcatttgagtaatataaataaaaagaacaaccactttgctggatatagttttcacagattacaagcctgatgatcaatgaactgctttcccattctcttctgtagctggatatgtttctgatttgaatcgtatttcaataatcattaatatcctgtggttgagcaaatcatttcaattttttccaaccaaaatctacacttgagtgtaaacaccaatggcaatgattaatgagcctggatttagaggctgttagaagatacaggtagcaaaatgtcaggtatcaggaaatccttcctcatagtaaggacgactcacaagacaactattgccaggaagatcaaagggaaatggtatcatgcgaggcatgtagaaccaacctgttctagcgctaaatgcaccagacagatggatgggctaatccgtcttcagttatctacaaatactgcgggggccacaaattgtttaaagccactgaaactgacattcactcatgcatgttatatttgaatctacacctcattattcccaaccactttcatcttattcttaaactagaaatgtaagggttagtgcattcatcagacaaactgataaaagacactatctggttagcctgtccagatccattagaaacctcaccagaagattaggtcttactttcagataggtagttatcaggtaatgaagtctctcctcaatgcgcctggctcccgagcgaggttggcacttcatcctccactacatcttctatgagtatctaaatcctctctaccctcttctgactcttctgtcctctgctggatggggttcagtgagagaaatgcatcctagctgagcagacgaaaaaaaaaacaacatggttgggcaatcagctatttgtcacactgagaagacatcacttcttaccaagcaagaagtagtagaatgggctgggcgcagtcgctcacacctgtaatcccagcactttgggaggccaacacagcaggagctcttgggcctgggagttcaagaccaacttgggcaatgtattgagagcttatctctacaaaaaaaaaattaaaaaattagatgagcagggtggcacgcatccacagtcccagctactcaagaggttgaggtgggaggattgcttgagcctgggaggtcaaggctgcggtgagcacagtgagcactggtcacaccactgcactccagcctgcgtgacagagcaagactctgtctcaaaaacaaaaaaaaaaaaaaaagaagaagaagaaggagaagaaatagtagtagatgagctacaaatacaatgccccaatggctgaaggaaactgaagtatggatgcagatatgacatgtcccatatatctaattttatgaaatatgggtatataaaatccaaacataaaaatcagatccataagactaatgagtgtagttaaaatttcctgaaatagtatttctaaatatatttaaaaactagcattggttgaacaccttcagttttccaataaaattgtgtaatttattatgctataatttttctttaaaacttttattttaaaagcccaggcttggcagcctggccaagactagcaccttatcttacacaaaatacagtgctctgcctcttaacaaatatagatgtaaccactgccagctgtttacaacaagcaaaagctgggaagccctattaggtgttatgtttgttctgctcgtgttaatagactcatgttcaatgtcagctcttcttcctagatcagtgtctgccaaatttcagttatttgtatatcactttcatgcattttgccatttaaagcaggtccacactcttccttatccaaaaccttagggccagatgtattttacagttgaaagttttcaagattaggaaagtaatatggtgcatattcccttattacataatacctctagtggtctgaggcagcaccccatagtcaaacatgtggacatttttgtagcaaaatgtctgaatattcacataaagataaatacaggaaaaaaaagtttcatgtgaattcaggtcaagttttgatgcaaatgtgtaaccctaaacattttttttaatttgtttttcagagcatttagaatttggaattatggctaaggtatagtattgtgagcctttatttttctttaattcatgcatttgcataaaaattaatatgcttttttgttaacaaaaggacactttatttcatacatggaaaactggtattcctttctataaatagcacataactatcaaaataaatacaataaatacaaaataatctatttaattctagcaaaccaaagctacagaagggttgtaactgggtcctgtatgatctcctccgtctctccctctctctctcttcccctataatgtgtctcaatctctctgctaaaaaagagattagcaagtattaggtgttaaagacatattcgcaccaaactgagattttctcactgatgtaataagaaagcctgaaaggaaaatggaaatgctttctatgtaattcaatatggtttaaaaacatatccttgtagctaaaaatcaagctacagcctaccaatggtagaagcccagtaccacctgcctagcttccatctatcctatgccaaccagcacaccatgtttgctcatgtccctggccagttctaccttctctgccagatatttggaaacatacaagggttgtagtattcagctggaaaaaaaaaaagttcagtggtataagaccacaggggtccaggaagtgcttcattttacataaagcccattttactttatgccccttaaatatgttttgcctttgtgcatggccatctgtgccgatgacactgttaccactagagtacctaaatatgagtgcagtgatcaactgtgcgtacacagcttcagtggatgcaagtatgatctctccgatgatcctgggtcccttcagaggcctttggatggttctgagcagtagaactcaaactgtgatcccaggaaggcagcatcagcatcacctagcaacttgttagaaatgcaaattcttgggccctaccccagacctactgaatcagaaactctgggtgtggagcggcaacatggattctaacaaggcgttcaggtgactctcatgctcagtgaagtttgaaaagcctgtgggtaacgagcacagtctctggaacctgcctgttgatttgaaggaatcccagatcaatcacctcctcacctgagactttgaacaagttatttaatttcagccatgcttccatttccgtttctgttaactgggacattaatagtacttaccttatagagtcgtggaggattaaatttattaatacagatagagtacttagaatagttcctgaaaatactgcacaaatatcaatagtattccatgtaagctacataaaacctttacttacagctaaataatagtattttctccaaataataaagacaattaataactacaagcttcatttgatgctcagaatgaggttcataggctttgtctcctaataaagtggaagtgtggaacttcagcagagacagccaaatgtcaggccacaggccagatgcagactttagacagtttccaaatagtgctaacatttaaaaatgttaaaatatcacagaaatcaaaatttctggttccccttgaaaaatcaggacctcagacaacactactctaggattccctagagctactggcaggcagagtggaggagcaaccacctcctttgaagaagtctcgtctcttgcagtctcacacaaatgggcagttcagttgcagtcatgtaccatcttcctcacacctatgacacaggggtttgtgagccctgtccttctgcaaactctaaggcctctaagtgtgtcctctgtgtgcaggacagggtgacagctgtcccactgggaaagcaagggagttcccaagacactggtctttcatgcaacaacagggagagtcccaggaaacctgggaaaaaatggtcaccctatctgtgcagatggttcccaagtctacaatccaagtgtctctcctatctggtccctgtttccaagtaccatgagaagcatttttttgtttgtttgttttttgttttttgcgaatcccacttttgcctcaaaacacactttccttcacgtttatcatgtgctattcaagcacttcatggatgaaggcctcctctatagcaggcacagtcctggacactggagatgaagcaacgaacaaaagagacagaaatctctgccttcgccaggttatgttctagcatggaggagagagacaatgaataaataaggcagagggaatgttgaatggtagtaagtgcctggagaaaacggaagcaaggcaggaggacggagagtatcagggaaagcaatgaggtgcccatcaaagtcctccctgagaaaagggacatctgaggaaagacttgaagagaatgaacaattgagccatgcaaatatctgatgcaacggtatttggggcagaggaaaacaggacctgggccttgaggtgggcgtgtgccaggggtttccaagtgatcaagaagagcggggtatgtggtgggagggaagtcagacaggccttgatgaggtcaaggaaataatgtccaccactgagttaagaccaggctttcctctgaatgaatcagggagccattggagagctccaagcagaggagtcacagggacctagcttacttatttttctcaaatagccatcgacatctagttgttttgacttgttattcaaggaggtcagtccaaggaaactagattccccaccttctataatttcagcatcccctctcattgaagacaggaaccacgtttaacatcaatgagtcctgtgggaataaagactgacagggaaccaaacagcaaaagggaagacttacctcagtttatttatggggatttatatactgcaaactaatactctatttcactggaatatttgtgtttatgaagacatcctgatgaacgagactttttttttcagttcatgaaattttcaccataaaagaaccctgaattcctattttgaaatccattgtgccatttttattctgcctgtatttgaatatagcatcaatatttaactactcattaaatgaattgtgacttttatagtgaaaatgtcagtctttgcagttcattagattcatttaaagcatagctgcttgatattttttttccctggaagaataaaagaactgtgaattgaagacataaactttaaatttaagcatgtgttttcttcaaaattaacattcaggatctgtaacattattcatgagattgaggggctgtttgcatctttctgcatttacttagtgactgtcgtcttttatggagtatagagctgtttatcacagttggatatgaccagtggaatgtcactgtggacattaagatgccataagctattatgaatctctacaattctgcatgtagtattaaaaacattatcttgaactatggcatcagaaaaaataaaccctggaagatgtcaaatttaataactcactgaatccaatagggcctaaaagtggtctatttgagttttaaaaaataattctgataagactttagactgcctgaaagaaaatgatatattaaaaatggaatttgagaaatgaattcaaatgaattttttagatgtttgcaattctttcttcctaaaattactatgatttacatgtatggagcagtaaatgcaaatgatttccaatcaccacgggtacgtgtactcaattacaaagtctcttaatgagcattgaaaagatttcagcattcactgaacaagacataaagcaaatgaactcttcagtgccctccccttcccacagagggctgaaagggactcactcttgttacaagaacacggccactcctccctctcttcctcttcccagtccatccctcaccccagtttcagccaccatccaaaaagaaatcgcagcatggcccttgaagcacattgagcttaaaagtgcctagaagataaagcctaaacttcagcacgacaggcaagacctttcaaaatctggcctcgaccatcctctctgggtctcctttctgagaacccctattttctgccttttcacaactccacacttatgcatgatgctgttttccctccctggctcacccaccccagtgttcttcttctgtgatggtaacctccccagaaagtctcttgagctagagtgatcattattaaattaaaaaaaaaaaaatcctactttgttctattctgatttcaaaaacctgaaaggcaagatatttgagggaaaaaagagaatattgttttgttgctttttaaaaaataaatttgcaggtcctgacaatcccgtgctttgtgtgtgctgatcacactgcaatctaatcaggtacagtaaataaaatgacttgagaacctttctgacgaatgcaaaggctgatgatattttgtgggtggagggctattcatgtatcttctttgagtatactaaagatccatttaattaagtttggggaggggggttctctatcttttcctctgtccttttttcctgaagctctcctactggaaagggaatggaagaaaaccttatgtggagtccggagtgcctacctaaattactaagacaataaaggacatacaaaagaagataatcaaatgttactttgggtacttgaacacttgctaagagcatgcatcctgcagtcagtaacattaccatctatactcagagggcaaacgctaatttcaaatccagagcaatgtcaaggatttatcactgcaacccaaagtatctttgctatcaaagacagtgggggcatgaactacaaaggcaataactaaaaagaggggctcatctcacacctctactcctctccaatattgggaagtccatttgagttctagagagtgcacatgccaaaatgcaagtgcgtgcatgcctgcgtacacacacacacacgcacaccattccaacattttcccatattttgtccactccctctcttccttgttatgggtagcagcagtgataatgcactttaatggaaacctgcctaatgaacccaaaaggaaaacgattaggaaaaaaatatgcaatacatcgcattgaaaggtgcccataattaaattacttgttaccgtttttaaatagtgcaaatattagtatgcccacaaagaactaaaggtttgtttgttttaatctagatcttccagaattggctgttcaggaactgcccatgttgcccaaagtagctactttttataagaatttcccactgcctttgaaatattctctgacattcccattggcttaagtattctcatacttttctgcctaaatttgtagtttcccaagtttaattaccccagaacaatctggtcatcaataaccaaataaaagaggatggtatttttttccctcataaatctattcatcaagattcgaccaagcctgcctggaatgtccagcgaaagcttcaataatcagggagaaatgagttgcagaaactcaaagagaacagaatagccaaggcaggagattacgaagccaggagatgagggcacactgaattaaacattcttaagcagacttacatacataatgaattggtcaattctgtccaatgtttctgaaagagattacccttccaacaagccaaatgagtgttcaaggatggtagctcggatggattcctgaacaaaataagtgttaatcactttatctcccccaatcttttttgtgaagaagagcaacttttcatttccataaagccttgaaatcgttggtcggcaggggatgggggtggtgggggagggctggttagaggatacagacaatgaagtccctgaaaagggggatttcctgacaataaacccctgatcagagccagagaggggacattgttgtcttcagcactcccctctcctttgggaattccaggcgtagtagagagaagagggtcacagtgacaactgcttgggaaggctctaaccaggtagtctttgcgattttctgtttgggaaaagctggcctacaacaatacattttcttggaatgtgttcttttaaacaaattgctacagactttcttaagacatcctgtcaattgttccaaaaatgtgtaatcagcttatgaaatctttaagcaaagaacattaaatgttagcataaaatctcataactagttatgtattgagattctcctacacaccaggcacagtgctgggcactaaacacatagcagcgaacaaacgaaacacagcggtcactactgtctggaaatcacagttaggaaagacaaaactgcttcttaaatctctgtgcactccatgccagacacagtagccattccataaatatttgaggaatgaatgaataaaaagacttcagagcttgaggttcatgtgactcaagatttaaaaataaaaaaataaaaaagtaaaagaaaagaaaaagctagcaggtatactagaaccaaacacacaaaataatgagccagagtgtgctcccacaatattaggagaaatggaacaagaaatcaaaaccctcgacatattgctcctatttattattttaatatcatcgttgcacagaaccagtcattggtggtgctttaacaattcctcctaaatagctttgtgacttagtccaagcacccagagacagatgagccccaattcaggcaagtcccctgttgaaggacaactattttctcaaatgaacaaagatcttctcttctcaatccgccagatcacacagctttataagtttgaaaagtctttttgctaacctagaggtattctctaacagaaagagggagatggaaaaagaaaacatctgcaaggtgctgaaacacgcgtagattattttacctgatccaggagcctccttccctggtgactacctactcctcagaatgagtgctggccttcttagctattctggggaacacaatgatggaggctccttttagctccatcaggccctagagaaataactgccctatatttagatagatgccctttgtaacaaaggaataattcatcccagttagcactttctaaatgacagaaccagaaagttttcgaaaatattcttctagctcagtggctctgaaccaggggacgttcggcaatatctaaagacatattttgttgtcacaactgggggtgctgctggcatctagtgggtagaggccagggatgctgctaaacctcctatgatacacaggaaagttccccaaaacaaagaactatttagcccaaaacatcaacagtgctgagatgaagaaatcctgcttaagttatacattaaataaagaacagcagggcatctactggtgaccccagatagttctgttaaaatattctgaagtaggattcttctcccagagtctatgttcagcaacttaaccagatgttaaaaactgacccgatcttaggatgttaacaatattttaaaataatactgatggtaatggtaacattaatgaaatgcttggtatacagagagttatagtgcatatttacaatgaccctatgttgcaaatgttattagtgtcctctagaaggtaactgtattaatcacatacaataactctatggagtaggtgggtttataaccccctaggaagtgtgtgctgtgggcaagacttgatctgttttttttttcaccaccatctgtccagcacccagcagagtgaaatctccatttcagaaatgatgatgctgagaggctcaggaacatgccagagatcacagggccaataaatggtggaaccccagttcaaaccgggcagactgcctggttccctagcctctctctctcgcaccgtgccagccggccaactgcttcatatgtggagtcattgaaacctattagacctcttgagtctaaccttagataagcagaaccaacctaagggggaaggaattaggctgctttccaaagttaccaaaagtttcccaatattcaagagactgattaaacaaaccagtgaccccaagacaatggtgaagtacatttgtattgccatgagacacttgaaagcaatctaagagggtggctggttttataagaggttaactgcttctcagtttgagcaagactcatgtggcaccaggcactaggcaccgggcaggcttcctgaagctaaaatggccactttctcaaaggacaaatgccctgcatgagtgcagaaactctttgcaaccactagggagctaactcagggatcatgggatttacatgtggaccagacaacaacttggccaccagtccaatggatccccagtgccaagttaaccttagcacagtcttcgggtaaaatctagatcaatgctgattatgtcaggaggtcaactttcaggaattaatactttttgaatgataagagagtgcaaacattaatttaaagtctcagagagtatactaattttcctgttttcatcatttcagtagattctataattctagggggaaatatcccataagaaataaattctagcaagtactgtaaataaagaattgtatccacctttataaaaagactaaaagaagaagagataagagcttgttttttagttttacaatattgacatagtaggctttatttttttttaaatgtggaaatttcccttttagaacgataagaatactagcctggacactatacaaacatagtcattgtacttataagtgctttaagtatttattcatagcctgcctttctctacaaaggaagtgaggtgacttttaaaagcatatgtgatttttaaaaggtaagtaacaggtgaatcaaagcaaggaaaaagtaaatttgagaaaataataaagcctagatggccagcacacaggaatgcatatcctactgccctagacagctactaaatgggtttcctgtttgactccaagcttcttggccatcaatgcaaagaaagaacaagaatggtaagattaatgcccagtaagatacagcgaagtggatttttttcagttcttcttgatatagtaacctaagaagattctctgtaaatcatcatttaaaaagacaccatgaggtgtaatgagctatacattgtaaatagaactttttaaaaaaaatgatgttcacgaaactattactcataatcctctcaatacaaagtgaagccttaatcagtgtgcaattcaataaagcagagagggcctgtgaaggcaactgtgaagtggggtgccacataatatgccatcgtgagcattcagctctctcacaagtctggcagaaatctcttatcaaattaggttatctggatgaacggaaggaagcaaggcatcttcttcatgccattctccttagatatattatttcttccaacagattttattaacacaaactgggcagcagggacttcaagaaggacaaaaatcctgtgtcaaaaccattacatatgttggcaacatttatcatcaggaaaagcgtcccaggaagttgaattctaatgtatgtgtggtgttacaatggaaattttagtgtgcaactggaatcaggccatgagttttgcctgtcatcaaatgtatctgttaacttagggttgaactgccccccaccccactttttttttttttgagacagtgtctcactctcttgcccaggctaaagtgcagtgccatgatctcaacccactgcaacctccgcctcccaggttcaagcaattctccggcctcaatctcccgagtagctgggattacaggtgcctgccaccacgcccggctactttttatatttgtagtagagacggggtttcaccatgttggtcatggttggccaggatggtctggaactcctgacctcaggtgatatgccctgcctcggcctcccaaagtgttgggaatgtaggcgtgagccaccatgcctggcccctcttttttttttttcttttttttctaactacacccttctctttttcctctctacaactgaatggtctttctccctgatataagaatcttctagatcactcccatccattgcattcttcttggtcttctgttatcactcttcttactcatcttactagcttttaacttgactacgttttcctttacttggtttctgccttggacttccacaagcttcaccctaataatcatgcagccttgaaaaacatggaggaaggaaaatgctgtcacctgccatcttgtacctgtttaaagctccactgttttcagtgtttaaactatgaagaaaggctccgatgatttaagctgtatagtcaaaactacagtgtcttctaggaatctaggattgagacaagatgtaaaaagaggaacatgtaatatattattttaccccactggcaattagaaatcgttagccaaagccaggattggggaggatattcgaaaagttattaatgcagacagagcaaggcttttatgccaccaagaaagttatttattataaacaatgggttttgagtcacatggacaatagctattttaaaaacaacgaatatatccaggaaaggtgcaaaaatataaaggcaggcaatcctagtttgtggtgctgatctattaccactgcaatgactgtcaatagtgaccctgagcagaactcaaacagttaagaaaactcaccacctgttttccaaggaattttatttttgcttaaggtggtaagtttgatactaaatacggagggtgttcaatgtcaagttgaggacatagcaatttaccctgcaggccagtgttcctagtgtctactattcatattagattaacttaagtggtgtataaaaaactttttaaaagtttaatagctatgcatttattgtatgggtaatctttacacattgcaagcaatacagggtcgccatatgcaatggtgatacaaattaaaaagtgggtcaatgtaaaaaattataaataatagcagtgataggtagatacagcagaaatgaatgtggcaaatctgcgaacacattgcaagacactggaagtgttcagaaggaaaatggtagctgtctaagctatcctttagaagattaaaccagaagtaaagcatagattccattgaaggtgagaaaagatgacaagcagggagagaggcctgggctaggatactgatggctgaaagaaaagaaaaagattatcagaagcataataaagaagacatgaagactccctggactaagggaatgaaagaagccaagtaatctaaattaattttgaagttttgcacctggaaatacggcactgaaaaaaacagaaattcaaaggggagtttggggggcagtggggatacaggttgccctctggcttatgctggatttgaactgacggcagacagggaagcggtcatagcctggagagagccactatgcaggagatgagaaatgtagatcacagtagagatgaggatttgagaccctggggtaaattgcaaaaatggccacaattctccacacctctgtctatccatgcccttctgtaatgtaattttgtggctcctcccatcaagaaatggattctgcgtcctctcccctagaacctgggctggcccagtgacttgctttagccagtagaatgtagtagaaggaacagtgtgccatttctgggcctggactcaagggttcttgtgtactttcctgctgtccgaaatactgccatcatgaacaagcccaggctggcgtgctggaggtgaaagatacatgccagagaactgagggaccccggtggacagccagccaacctcctgaagcaaaccctccctgccaatctgcagttgactgcaaacacataagagacccagaataggataagcactgcccttctgagcccaacctaaattgctgacatgcagaatcagaaacaaaatatgtgtttttgaagtcacaaagtttagaggcggtttatcactcgcgatagataactggtacagacaacacgggtctgcctagaaagtgaaaaacatacgaaggcaaaacagtaagaggtcaagctttaggaggatattcccaattcctaggaagaagggggaaagctgtagacaagacaacagggcaatggcaatcagaaacattcaagtatataatatcagggaaccaggaacatgggggcaggggacacagaggataaaagtgggggtaggagaagtcaaggactcatcagggaagtatagcactgggcttagcagagaatctgcctagagaaggcactggtatttttgcatgaatgattgaagtttgatctgtcaaggcagagctcgctggagaagagtgagaagacagtgtcagtaacgtaacaaggaatagaagtggtctatggagtgttaaggagggcctgggtgggaaagatatggaaccctaggtaaagattacttgtttgaagaatcattcagagagatgttgaagacgaggtcaacagagttggacaaaagtgtaaatgtgaatggtcctttctctaggaccctacaatcacaagtgtggctgggaccatccatgccagcatcacttgggaatttgttagcaatgcccactctcaggcccaccccagacccactacctgtcttttatccacatccccatgtgccattcaagtttgagaagctctgctctaggaagtacaaagacaaggaactgaagagcatttaatgacatcttgagaaagtcattctgttttgaattccctttcaatcccccagataaaataacacaaagtcttgccttggtactaagccatcttcgacacctccctaacacttgcaaatctcccttttcaacagagttcaacagagagcagagccttaggcaagcaaggtctccagtgagatttgaaaaacactgtttacaccccttttgtgtttatttttaggattttcttttatgtgtggaaagcaatcaggttttccatttgcagtagtcttaatttcacattaatttatttaagagaaaaaaagaggtaaataaaaatatgtagcatataataatgtggatgcaaatgagaagacagcaaaagttgttacaagtgaggtggcactcaaagcgaagactggaaaatctatagtggaagtcagaagacatctgggcacatttgaaggtagaaaggcaagagacaggaaagatgggagctgtttgcagaacaaagaatattcaacaggcaggaaggactgttggaaggatttatcccagagtagaagctgccctctctgggcctggaagaaagaaagatctggtccctgaatgggtgaggatgacaaatcagagatctaaggcagataatatcaaattcttcataagtaaaagattggaggtcccctctaaaatgagggtaggttggtttgactgggactgcaaaagagtatgaaagacttggaatatccattgtggacaatgaccaagagaacaaacagatgaaaaggattccgcagcaaaggcccagggacagtcaggcagcattaatttttagtaaggaaaatggacacagtttatcccaaagctgagaaatgaaactctaaggctgacacaaaaaatacaacagcaaagaaagggggcagaggagattaggtagtagtggcaagcatagctgacaagtcctcatagtcaaggttacatacaaagtcaaaggttggctaggtgcagtggctcatgtctgtaattccaactctttgcagggccatggcgagaggatcacttgaggccaggagttcaagaccagtctggtcaacatagtgagaccccatctctacaaaaatattaaaaattagctgggtatggggtgtgcacttgcagtcctagctactcaggaagctaaatcaggaggtttgcttgaacccaggagtttgaggctgcagtggctatgatcacaccactgcactccagccggggcaacacaacaagaccctgactcttaaaaaaaacaaaaaagagttaatggaaaaggccagtagactggactctgggaacaagattctcagtggatatagggatataagaagtgagaaataaactaggtttgatcaaaaaggaaaaacatcatgtttttaaaaaactggtttttaaaatgtcaactaaatattaactagtgttcaaaattttgttaccaagagttgggacttgataaatggcactacaaaaactcaggaaaaattttgaaaagaagagaaatttgggcctaaaatgcaggtaatcaattaaaattaggacataatgatgtgtggttcttgcaaagaacataaatcactgggtattttaagtgtgcccaaaataaaatcccaaaactgaagaaaggcctgaaaagatataaatcttattacagtgtgtctctctcaccaaaagaggatcatccaagaagcacataaaaactaaaactttgaagtttaggaaaaacaagaacactacagagtatacataaaatttcaaacttggagatacccaagaagggtttctcagacctcacccatcgctcactcacacacagcacacttgctgaactacctggtgtcccttcctctccactcactccttccacagtaattgagagtttcactgggcacatggccagccacaatataaagtacatttcctgagtacccaagtgtggccatgtgatgaaggctagccagtgagatataagcaaagtggcatgtggcagcttctggaaacctccgtttgcccttgatccccttcaatccccttcgtccttcttgctggtcaaaccatggaggttgactagaggtcaaccttgagccataaggataagggtcacaggatagctagaggcagcttggctcttcagtgtttaagaagcctccatactagccttggactacttccctctagatttggttagggaaacaagaaatgagtatctacttcatttaagacgttgctgttcaagattttttctattacatgtaagttgaaccctaatgtaccctctttgctattatgggggcctcctattccttgaccatttcttcaatggcctgaatagttggattaaaagcagtcacttcaataccacatttccctcaactcctgttctctcatccctgcctaggcaaaatccaccaccaagtcagcttcataatcattgttctcttcctgccaccaacacatcaaatactgataaaaaaaattaaaaataaaatgtaaacttaaacacttctaatagggatcatcacaactgtagtccaactccttgaaaatggacaagtgacttcactgctgcattcctattgcctagaagtgtctgacccatactatgtcaacacatattcactcaaatactaaactacaaccagatcttctgctcaacagtatttttattcatctctagttcacttcttaccacattttatgtgccaggcacttctctaaatgtgagatgtaccatgtgcatcaaagtaaatacaccccctatcctcatgaagtttacatttcagtggaaagagatcaacaagagcagatgaataaatgaataatcaagaaattcaaatagggtacatgctatgaacaaaattatgaaggtagagagatattaattagagagatggggaaaaggatattttagaaattgagatttttttaatggtcaggacatacttgaaggtgatatctgggctcagacataggtgctgagaagaggccagccaagtgaagacatttggggagagggctctagacagagagaatatcaagttcaaaaagatgggatgaaggctggtaccgttaaaacacataaagcgagggtagaggggaggagtggtaagaaaatgaggtaggaaaagtacacagggatagatctcaggcttgtcaatcaattcccatgcccatacatattgttctctctctctctcctctcttccccaacatcttttctactgaaatagatatacactatatatatatatatatatatatatatatatatatatatatatatatatacatgtgtatatatagaacaagatatatttctcactctatatccatacagccactgagaatcttattcccatagtccaatccactggcccttttccttaactcttgacatttttaagagtcaaggtcttgctgtgtttcccaggctggaagtgcagtggtgtgtgtgtctgtgtgtgtgtgtgtgtacatatatatgtatatatacacatgtgtgcacatatttacatattttatatatatatgaatcttcagccatcttccctttctttcctattatcacaaagaaatagttaccaaggcttcatttaagggctaactctcctttatctacagtgttcgttccaacagcaactgaccacccaatccatcattcaatagctctctcactctctctctaatctcttcttcaaacatgttcagacattgtcttgaaaatatcttctcattagccccacttcaaaatgtcaccctctacctatacacacatatatgtatgtaaacacatacatatgcaaatgtaccgagacacacacatacacagtttactagtacctactcaatacaacctaggatctttcaccagacattctagtgtctccacagtttgttctcaaccaaccacatctcccaccatggaaagagaaaatctcatatccatagaatatccaggtgacctataggtttcttgaaggcagaccacatccaatctcagcctcaacaagaagggcgttcctaatataaacaatatttatgatatttacattacataagcacaggctcattttggtttccacaaagaataataataaacatagacaatggaaatcctcagaaatcttcaaaatcatcaagcttcactccagttagccgtttcaatggactttgctttcctatcctacacccacattctctcattgtgacatcagcactttgtttttcctccaaagaaccactgctccatactcttagctcctgtggcttccaagaagatgacagtgtccagctcccccactggtatgcctacgtggctacgagcctgggcatgggacccaagtctatccaatgagacttgatccagaaaagctgctgaaacatgcaagaaagaatctctccctcttgaagaggctgagctgtgagaatgtaacttggagctgctgctggacaacttcccactacttgagtacagcctggctgatatgataaaaaaaaaaaaaaaaaaaaaagaaagaaaaagaaaaaaacagatgaaagcagagctaatataagaaagtcttgatagcatcccttgagactctagatccagctatgcctgaaacaaaacttacaatcccttttctatttaactcagtttgactgaagtctttttctcacttgcaaccaaagcatttttactaaaacaatcattttaaccacttttccattaaacatttttattagaatttttaaataagtcaaataataagggttgaatctctcattaaagataagttggcatcaaggagcagcacgatggatctaccgagtatattttattaactgtttgatatttctagatttaactcatccccttttgaagaagatcaacttccaaccaatcatacatatttaataaaagttttctagtcaggtcagcattttgaacttctacttcctacacctaggagattttttaaagaaactgtagaatttttattattctacagtaaatcccttgcttattacaaattagctgtagaaattgctggaaaaaatctcacaatttggtacagttatactacatccatttaaaataaattgaattttaaaactcactcttatcaccatctttgacataattcttctgcataaacaatttagtcaatgtagataaggctacattaaatttgcaacatttggaatagaaggacccagcttaaaacctcactcccccaaaagaatgagtatatatcattcaaatatgaatttagagtaaaaactcaaatgtgtcttcatattagcccatttgtctatcttcaaattcttcttttacaaacaaagcaaaattgaattaagattaaagatagaatgttaatcatacaagaagggccattttccagggcctggcatgttttacacaagcatgaaacatatttaagagaacatggcctccctgaggaagagattaaggatacaaacctaggaaacaaagaaaataaaacgatctacctcaaagggataagattaacctgtgagaagctcatgcggaccttaaataaaagcctctactccttatatttttaaacaggatttttcttttttttttagagcactaattccaccttcaaaatatatttgagtttgtaagtatactcaacaaaaatcctgtcctttcttattaaccatactcaaaacagattctctaaaatagttatgacaccattaacatttcatgaaatccaaaacaaatgtgtcttccaaaaaaataaatgttttcattcataaatggctaatatggttttaaattatgttatcacaaattttgcatcattcccccttaataaccatttaagaatgtttccttccaaatagctattaagagagaaaggatgtttcatcatgcatacacacacataaacacacactccttgtttatgtcattgtttttaatggtgcagtaggatttccaaattcttcaagtttcacacattcaagaatgtttccttccaaataactattaagagagaaaggatgtttcatcatgcaaacacacacataagcacacattccttgtttatgtcattgtttttaacagtgcagtaggatttccaaattctctgtttcacatgttacttcctaaattactgcctcactgactgggaagttgaattggcaacaccagccaatcctcaggtccaaggatgttgtccttgcatttcctgtctccttcctcttgaaggactcaaattcatttcattcatctgctttactacaaacagaaagactccatctttggttgttcagccccaaactctctcttgagttccagtgcatgactctgtgagaatataccactgaaatatccaaaatgagcttgatctggttatctttcctcaaaaccagcttctcctctctccgctctgcttccactcaaggtcttaccattcgtccaggccccaaattcaaacaccctcaatcgtttctgcttcctctttctgccttccagtcgggaggttctgtttagtgctttcttttcttttgcatccaaattcacatctccttgattcaaatccttcccataccttacctcaactgggtgatggcatcctactcccggtctctcctactcttgccggggcctacaccattctactcaaaaggtcagatcaatctttacaaaagtaaaactctaaccaatccaccggtccacttaaaatttttcaggctgggcgcagtggttcacacctgtaatctcagcactttgggaggtcaaaggcaggaggatcactggcggccaagagttcaagaccaaccctggtgacacagcaagaccctatctctgcaaaaaataaaatgaaaaagttagccaagcatagcgaacacacacctgtggtcccagctactcaggaggcctgagtagctgggaccacaggtatgtgtgcatctcttgagcctgggaaattgagactacaataagccatggtcactgcactcaagcctgggtgacacagcaagactccatctcaaaaaatatttatgtgtatatatatattcctcgaatgcactcagttgcagacaccgttgactttccaacagccttcttccttgctaacagagcataacactaattaattatgctcacgtagcaatgcccagcctcaagggatattggattggtctaaactgatcaaagtaatcttcctctttgctagtgagtagcctagggttggtacgcaacccatttctgaccaataagataaaagagacggtctagtggaaaagcttttccttcttgccaagagatgtacaggaagagaaagtactcatttgtgaccactcccaccaccatcaccttgcttcttattttgtgtgttttcatgtgacgatgtgacacccagagctacagcagccaacttgtagccatgggaagactgataaaactccaaggatagcagaataaaaagaaaatcaaagaagggctcttcaagacatgattgaaccatccaaaacaacctggcactgccaacccaaagacttctttgaaggtaaacaagtgtcttttatggtttatgccaccatttgttgagttttctgttctctgcagcccaaagcactctaactgataaacttagaaagagcaaactcttgaccttagaaggccctctgcttgatgtgctactcaaagtgtagtctacagacccacaaccttagcattgcctgggagcccagccctgggcccatgaatcagaatctccaagggtaaagtccaaaagcctgtgttataacaagctcccaggtggttcttgtgcatggtaaagtttgagaaacactgccctacaaaatgatcacaaattattttttaccatctcagctactaccgtacatagcatgagatgcacttgatctgcaatttgtctggtggcagtaacctttccgggtttgagcatcatttgttgctcctttagtttcttctcctcttaatcaaaatcttcaaacttgaagccttcaggtcctgagaggattacaaaggtctgaatatgagacaggctgtgaaacaggccgtgatgaactatggggtacaggcacatctgaaggacaccgagcagatgttgccatacaaggatattggttcagtgttgccagatgtctaaagaaaagctggaatccagatttatatgcaaagttttttagatatagacaaccaagccttttttaatactctgcagattaaacaaattttcaagtggattcacatccagctgagtttgtgacatgaagtagattccttgaaatatcgacacatactttgaaaactgtgaccacctacatgcattgacttcatttgaaatgactaaaattaattttctttggctaactatatgctagtctctactgagcacagggtcttcatcagtagcccagcatctggagtggtcaaatagtttgtggacctctagttaggagcagcaggactgttttatccattagtcactatacaccacacacttagaactgcaagcttctcatgggcccacaacaatgtttaaaatgtgagaaaaaaataaactggccacacatcaccagaaaactacaaaattacaactaatagatgtgaagctaaatatttacaaaatgtaaactaagtcaacggatccatatcaactcagatatggtatcatatcacattagattaaatgcaggatgtggatgcattttaatgtttgatggggcatggaagggagctcagatcaatagtgcttagagctgccaaggttctaaaaaggccctgggtaccaacctgaagccaaccattctgtggtaagtctggtcttcacaggtgtcacaaatttctttacgcagctctactccattctattgaactgtctacatagttggacatgggagcagaactgtccacatagttgcatgtaaaccataaatgacttattaaaaagttatgtttggatcctcatctacatatgaatttaagacaagaagtacaagaatcagcacacaagcaagttttgagggtcccacataatgattttaattattttttgttaatttattccaacatttaaaattcatgataattcacataaaaacccagattatcatcctcttgaaaaaagaattgtaaagtatagagatattggacccaaatgttggcatggcaacactctactaatgctgacgaaggctatccctttaaatggggtatgttctctacttggccgcaaaccctgtctccatgtaggctaacttgctgtcccaagagacaattgagtttatgccccctagtttaaggctcttaggaaaaccttatttctttttcacaatgtggcttcaatatcttacgtgaagacctgtcgtgataggatacaggaatctctcaatctaagaagagtttcaatcaaattccccccaatatgttctcaatttgaaccatgatccccaagaagattgacaaatgacaaattaccctgcagtttcttttgcccttcaagctagccaaggcagaagcccaatcaggtggagtcaaagcactaatataagaatgtcttaattaatcaaagtgatcaataaataatacacataataattagaaagcaaagtctgggactgatagaataaaaacaacatagcactaatctaagtaagggtgagaggtcaatagccataattcaaaaggaaaagccctaaaaatatgcagaaatagctagtaactggagtttagtccaagtttgttaacaacccactgtatgatttcacctcacacccacattaaacacagtatcttattttgggatggcagaattctgacaactacaggttgagtatcccttatctgaaatgcttagatctagaagtttcagatttaggggttctttggattttggaatatttgcattatacttaccagttcagcatccctaatccaaaaatctgaaatccgaaatgttccaatgagcatttcctttgactgtcacatcagtgctctaaaaagttttggattttggagtatgttggatttcagattagtgctaaattatatttgctaattgatattctgtgtattgttttcttactggtgaaaagacaaattgaactcaggaaagaaaaattacaaatatatagtcaaagagtcaccagatcatttgagcaacataatgagtttttatttcttaatatacagggtttaatttgttaaccacaccagaaaaagaaatgttcaaacagtcgactttctacttgaaattaaaatggaaattagcccttaggaaacagtgatgactttaagtttatacatcagtggctggttcatgaattattaactgattttaatgatcctggctatattttatttgaagttattctcctacttgcaacgacttttgaacccacaaaactctttcatccattctccacagacacagtacactttcatgagcactgacatttgaaaaggatccatatactcacagaatgtaaagataatacacatgcatataattgatacaaaggactctggtcatttgttaaagagagtaatgaatgaaaccctattattcacggtttgtaaataaatttaaagataagcgctaactttcacttgttaagcctagagccaagagttcggtgtttcttctaaagtttcccagatggtcattaaaacaagagagggaagaaacatactttctttgaaatccatcatttcccatccagaaagataatacatgtcatcaaaagttaagtctttagagattaattaataatgtgagtattttcaaataaaataaatgcatgtctttatatgctagtttttacattttcagtataaacctatattgaacagcaccacttcagacacacaaattagctgtgaaacaaaatgctataaacctttaattagcacttgctgctttatacccataaagttaatacttcctcttctactatattctctgtaaaggaaaattatgggaaataaaattgtgtttttcacttccgaaaataaaagtagcaaaatatagtattgtggctcagtttaaagatagttatatttttacttgcaaggaagtcaatacaaagatcccttacaataagtataatttgattcctaccaatattaagcgctatgtctaactttcataacttgggtacacagagcactgatgacgatcagattgctaaataagtaagaaagcaagttatttcaggcaaatagctgcattaagaaaatacgacagtaagccaacttgtatccacactcctgaagctgaacaagcatctcaataaataatgtctgttttcaagctttatataccctccctgaaataaatgttctgagcttaaatagactcaaaatagcctttctcagaaaataaaaaagagacacacacaaaacacccccaatttcctctggtcccttgaagtacccttgggatcccagtgaacagagatttgccacgcattgcagtgactgcatttgtacaataggtttctattttggtgtcattcgagtgttgcagatcttaaatcagagaaagaaggagttacattaggcaaggtaatggcttccaaaaataagagcatgcccagtttatacaaggcttgcatccacatgctaagttgtaaggagataaataataccccactggaacaattaggtagattctcagaaacactacatacttaattaggctgcctaagtcccttactgagcatacatgggggactttttaaagcatttaataacctgagtaaatccaaaatcagaggacacaaactaatccttaactctggctccagcgatcggccttttcttgcctctctataaatgcttagcgtagcccttagaattcggagagcaggaacgctccaaccaccaaagggttaaagcacacggaataaagtcccctcgatctatattttttgggaggcccctctgtgccagacgctgagctgggcgatgatgacacgcgagggcgagcaagagagaccctaatggtcttttacaaacttcagcactagagttgggaacgcagtataaacaaatagaaaggaacacgagcctccatcagccacggggcacagcaaactgttcctcactctagatgcaattcctcctcttcctgttatttattcattagttttgtttagcacctttctttcagcaggctccttatttcctcctgttatccaagcaatcacaaacaaactacgatttgaaagaaaaagagagggaggagggaggagcaggaggaaggggaaagaaaaccacattcaaaaggcacaggccattcagagcccggctgtttctctgaatctcgccctcagggtgatgttttcgacaaattcggcgctgggggcttcagggaggacagaggacaccggatggtgggggggaattccgcgcggcccgggtaccagggctgcgactctcgcccgcctcgctgctccgagcgacgcccgctgcccttctgaggtccacctcgccacctggaacgctcggtcctcgaagttggcgcagggggaggtggagaaggtggagtgaccccagggtgtggtgtggaggtctccaatcccgatctccccgaaatgcaaaacttagcccccccaaaaaaatgagaggaaagaagccacttactctaagcggggaggggcgggcaccccggttccgaggtccaaagtgccctaacgaaagggcagcaaaggccacctgcggggtcccaccacttccccccactcccctgtccaggcatactcaaaagtttgttcctttccgtccgtaagcggccccgaggccggctctggagtaggtgggggaggtaagaaggactgggggtgggggagggcaagatcatcggaccaggagatcagggacccctgcgtagcccccactcagtccaggggggctgcgatcgtgtttactaataacagcaacaaacaatcacacgccagcccttctttcagtcctcgggctctcttcgctttttgggggcagcaaaggagagtctggcggggaggtgggagggagcggaggggctgaggagagcgccggctgggggacaaaaagcggcgtgggatggggcagaaagagaagcggggagagctggacagagacgaaggctcaaaggtagaagagacatgggaggaaagaggccaaagaaaagtgcagcaggcggtgagcccgagcgcggggcgcgcccaggatggggaggtggcccccgccccccggcccaccggcccagcccggtcaccttgctgtagccgtagtaccccaggcactgcgcgaagtccaggctggcagggtccccggccgccgcggggtagaacctcacatccatgccgaagctgggcccggggccgggggccgggactggggttcgccggggccgggacccgcctcctcgccgccgctagatccaccgtcgagggcgcccgggggtggcgcgtgggactcgcggccggaggggcgccgggacccagagcccgaggagctcgggagccgcggccgccgcacacaaaggcgcggccacgcgagccgcgggagagcgggaggcggccggggggacgcgccccgccggggcaccgaggcagcgctgcgcgcgggccgggcgccgggggcgcggggcgcggcgctggggcccgggtcggcgaggcgagttcaggtgcgctgggcgaggctgggacggcggcggcggcggcggctggccccgctcctcctcctcctccccgggcggactgaggagacgagccgcggagacaaggggcccggcccctcccctccttctccccctcctgcctcagccgccggtcccctccccgcgccgccgccgctccgcccctcccaccgcgggcagctggcgcgccgcccgccccgccggtgcgctcctcggcccggaccgcctccgggagcgcccaccccgccccgccggccccatcacccccgccgcggccgccccctctccggggcccctcttctccctacctcgccccccttcccgctccggtcctctttgtccccactgcctcggagcgctcctctctcccctcctttcctcggccgaagcggacgccagccctgcgtcccgtgtgtcccaccctcctcctgcgcgaccacagcttccccgggccccagcatccccaagccgtctgctcccttcctgtcgctttcctccccatgacccccttccttcttcatgggtgcctgtccccgggcccctctcctcgaccccgccttgcctttcgaatgtcccctgcgtggacacacctaacccagacgccaacctgcacgagtgtctcccggtagccgggagccgggaccccaagttcacctgtaggatttcccagaaggggtgttggggcctgacctttcacaggtaggcagacttgaagcttcccaggtgaagcctcgtgtcggttctcttcgtattctctttgctcttgtttgtattcgaaaggcatttgagtaactggatctaggcagatcattcactgctctgggagagaaaatgtccctattttcctaaaaggtcctagctgattaacttccccactctacactcacccacccctctttatttctttatctttcttaagctccatctttttatttaattttttttctcatcggctttattgctttgagttaatctgagagtttggccttcagccttggcgaagggaaggggctttatactaaatgggaacgttgtggagaggccgtgagatctgcgccctccccagtttagttatctgccagctactttaaccagagtggcgacattctgaaatatttcctatgacactcgctaacagtttcatcccctggtcacaccttgctggcccagggcctgagctttttcagacaagcatgacatgggttttcttttcatttcttctgttctccagagtagatcgggcttgtgacacctgtgtgtgtaccttcactcaatattatctttctggaacggagcagaaaaaaataaacttcaattaggtgaaagtttgtgtatttgctcttctcttagcaaaaacttgggtcaggagactactgcatgagagagcagtgtgtgttgagaaaagaaacaactgcccacgttgtccctgggttgactctgatccccaccctcccgaaaaagctttggtttcccctccctagtctccatttaacgttgacaacccagcggtgctccctaatggcccagcaggttgcacaaattgacagtgagctgagatattatcaggacaacaccccattgagcaagttcttgaaacgcagaagtggaggccctttctctgggccactgggcgcctgtggcctttccaccaatcagagctggaggttgcagtggccacctgttgtctcgcaagagaacatcttttaagtttcgctattagtatttaacaatggcctacagagcaggatctggccacaggagaggagcagtgtgatgatctggggtaccaaaaaagaattttttaaaaaaaaaattttacagatagctcttacaaggagccaaaactctggaaagctaagtttttctttgcacactcacaatcctcaccttctgctcattctgttttgcatctgaagcaatctgagcttgattaaaggatgaagaaaagtataatcaaagttacagagactaaacctgaacttgtaatcagacttcgaggatcaggaatataaatacgcttcgcagtggaaacccgctagctgcaagaatgggaattgcaagtgggctgatgtactgcattcagctttgggcggtagagcctagaaaatgaagcgagaactttacccctaaggctctcaggagttaggaatgctcggtgacaatccctaactcctggccattgttttggaaaagtctttctcttaaatttcccttccctgttaggatgcaaatacctttgtgaagggaacaatactcatctgtctgcattttattatttgatttaggatataaggtaagcaaaagatctgagtttagattttgatttagagggaggggaagaatccgggtgatgagctgaagatgtgtatgttgaggcatttagaatgagaaaatcactggaacagaaaaagggcattggtgaaggctgctgacttgacgatttagcccaatctccacgcatttggcagctatctttatttattgttttttttttttttttttttttgagtacctaccatgtgccagacatgattttaggcacttgtgaatgagaaagaggattcaattcccgacatgacacatatagaagtcctcaaagaggactatgattttggctcttggaacacaaaatacacacgaactcacatgcacacgtgtatacatgccctgaaccatcccagtcacctggagatactgtcatggcacagccactagctgctgttgagagaagcgacatgaaaaacaaactcacatacagtgtgaaggatgccagcttctacacccccatggtatttgtataagaaggctcaagggatgaggcagtgcatcccttgctacatcattgctggatcccaagaacctcagaccatgcttgatacatagtttgtttgcaggaaataaatattcattaaagaaagaaagtttagaaagaacatgggaaggaagaaggaaaaaaacacagaatgttttaatagtgtaatttatttgcagggaaccttaaggcaggatttgagaagagtatatccaggacctctgatgtgaattagaacaatctactttgggtggggtgggggccttttgcaccctgctgttgtagacccttcgcagacataaaaactaagatcctatgtggtttcctttagaactgatctgattacactctccattgctttcgcactatcatctaattttaagagttccagtggttttgaagactatccattaaaaactttagaataatgctttaaaaaattaaatgcaattgtaactgtcatggtatttgttatcttaagggtgtcactattttcagtctgtcagcagtcttgtttctgaaagccatagacaaaggtttcctttatactggattagccttgaatttgaagaaccttctccaagaacatatcttaaatatgctttgaatttatgctgaacaatgctaacagttattgatttagaagggcagcagaagaatcaattctgcagccaaaatacaacttctgggcataagaaaattcctttagggttttgagatgattcggatcaaaactgtccatcttattataatgcttgatgtttctaattaaatagttgatgtcatccaagaatcccttaaaaatggggagaaaatacacaaactttacattgtgggaaaactaccttacatcttgacggaacctgaaaactttctctactttatctggtttcaaattatgcaagaaagtccccatgaggacttcattaaacctgccagagttttataaccaaaaacctcactggaacccccacttttacgaatctatgaaagtaatttaacccctctgtgttctgcttgcccaagatcagatgcaaatactattaaacagtatatacagtttaattctcttgagttttggtgatgaaaaatcctatccaaatattatgatccttgcaatacgtaatgtaaccaaattaacgatagctccaggtggaaattaacaaggccagggctactcacgtttcctctcatctattcccatagtaacagctaatgtattatattgctaaaatatcttatattcagttttatgtctgaacttggtgttgtgtcataaagcgtagcattcctatccagactggattcttaatatgtgtatcctcatgaaatttaggagagtttcgtgtctgcaaatcaacacttcatgtgcatagtctaaaccacatgcagataaaagcaagtcagaacctaccaagactctcaccatagtgaacctacacatggtatagaggaagaattcttacctgggagacctgatgacacattaccagtgcaggacctaaagcgaatcaccatagagagatccacacaggaattatagtgtgaaaaaaaatccccaatcatagacattgttagcttcagttttcagtgaatctaaactcttctagtatgaaaatgtgagctattttgatatttttactttggtcaaaaaaacacagaaatccaaaaaatacagaaatctgggaaatgggataaaaataagcaaacttttccttcatgataataaatatatattcagttgtgtacatgaggttgtttcccatactactacaattttagtcctattggtctctgtattaccaggcacaagaagggctctagaaaactaatcattttgtcagttgactattacttttacaaagaatagataacacccataggttctgacagatacatagtaattatggaacagtcacctccaacaccctggtagcaaaaaactattaagccaagaattcattaccacaaatatggaatgcagcaagtatttgcttgtgacctaagtagctcttcaataaagatattttagaaaccaaaacactccacacaatcaggaggtgtgcaattttcactgtatttacactctgccttaaaaaaaaatttgtatgttgtcctgcctgttttctgaaatatattctatttcttttaaagtgaaagaatgacctcttttgcaggaacgattttcagagacagcgtgtttatcaaatggtttctctgctttcataaaaacaatttattttcactgaactcttcacgataagcatcaaagccaggaagaatgtacaatgaagtttacaatttcactccagaaacaaatactgggtagttattattttgcttaagtgaaaaacattttttttccccaaggagacaaaggtagtaatgggggagggggtttatacaggagtgaaaatcaggaagtccagacagtcttcagcaggaggggcccctggtgttgtccacaaagaccaccggctgaacaatgaaagctgaagacccagtactttcttcgcaaatgggtgggggtcagtccacagttttattcttcgctaagagacagctatagaggcattaaggagagaaaatcattaggcagatataattaaacccttctatgacagatctatataccattgcacatagatatctctcatagttggtgctttggatgacaatcctttctttgataaacatccatgtttctaggcttaatttatatgacatgttattttttaataaccatagaacaataaaattgtaaaaataaccaagaaaagaactgagtctaataaagattctgtttcttaagaaatgggaaacaattgcatttgatcacagaacagccaaggactagcagcaaaataatctctcctaatttacatagatctgtgcgtgccaaacagatgagtgtttgcttttcgagtaatagtgcaggtgcgtatcctgcagtgactaaatctctaactcatcaaactgtttcgccagaatgggacatgccctctttttctggaacagtttcaagttttctccccagcattccagccacctgtttatggagttcatcctcgggagaagatgcatgcaattagaaacaagaaaatgaaaataaatgaccttgtttgttatttataacaaagaaactctgcaaaattaatgctgtgaagcttgacctcctgacccaggaaaaagagccacctcatggctttgtggccaatggacaatagtctggcagctcccattgtccagccaccctgacttttggatggtcattaaagccacatatccaaaatattaaatgttttacaagttagattagaggatttgcactttttaaaatgcgtgttttaaaaactgaggttagttagcacaagagattttctttttaaggacagcaggtaaaaatcatttagcttcatctactccatctatcctgtgtcaacaacatgttgaaggaaaggaaaagatttaaaaattctgaactagtgctttgggaattgaagaatggttaaatatataatagaaatcaaatagaaaggtggtctttgaaagtggttcagggaaaacattgtgtttcctccctgctgaggcctcagggaggaaagggggaattacaatgttctgttacataaatgcagtgtggatggtgccccctagagttgtgcgatgaaatagcattgctgtttttccttccacctcttatcccaacccacacatctagaagcccagggcaggaaggaaatgaaggtggtaatggagttttgaagactcttcagaactggtgcttgggtggaacatcttgaggtaggatggcgactgaaaatgacactctctggtgtggctgattctgaagctggcctctcctactgcatcttgcctctttccccatttggtctcctccttcattcacccacaacaaattccatgagtgtcctctattagtctaaaccttagagtaaaatccgtgtttgatttttttccatccaagttgcttatttattggccaatctgcccagtgctactacatctttaattgttttatttttccccaggaggaagtaactgtttattcaagaaggttctctactgttcctcaaaatgtcttcttcactgtgaaataaagtcagtttcataaggcatgcagattgtcatgcaaacaccaccctgagcattctagatcatacctcctcaccttcccttttctttctgatcccctgagggtagctcattatatgcccctccctaccccttcttgggaatgctgcagccataaatctggagaggtcattagaattacgaactctggttggtgtctcacatacctacaacagcaacaacaacaacacaattatagccactgtagagccaatgtcctggttaatgtttgtgtaactggccaattctcaggcccagagaaaggagtttaagaaagtttttgcatctcaccagtctaaaacttcttctgcagtccaaaaccacatttcaactattgtttaactgaaagactgagccctcttaatctggaaccaccaactaaattccccctgtgtgcttaaacaccaagttagagagctgtgttcagtgccagtgagaaaaagtagagtgtgattaccccatcttacccacagctgaaacctgtgtcatttatttttcaacaaatgctgactgtgcctttctaactatatgctcctacgtttccagaaatgggaaaaagcatgatccctgtttatactaaacatgacaagacatgaggaaagtagtataataatgaaatattgtaatgagtgccataggaaaagcattcttaggctagtgtagtcaacaaacatttatttacagaaattgttacaggtaatatgttagtgtgctgcttagaaaaggcggggatcatttatgatcaggataatcagaggaaacttcatggagaaggcatttgaaagtagatagcattttggtgaggcaaaagggatgaaaaaatacactagtggagggagattttgaacacagttattagagagaaaatattccatgtgtggtgggaattttgggttgcatcagccatttgttgggtagattaagcttcagtaacaaataacctttaatatcttagtgggttaccagcacagaggactatttctcagccccgttccagtccaccacccatcagctgctgttcatttgcacatcttcatctcaggaccaaagctgaaaaagaagcttctatctggggcatgtgggtcttgtagcagaggaaaaagagagagtggaaacccgctacagttgtcaaagctgctcacctcattcccgctcacttttcactgggcaaagcaaatcagatggccaagccccatgtcaatggggcagaaagtatgatcctcccggagggactggtctggcaaagagtaaggaggagtcccctattttgcaataatgtagtcggtgccatggaaagagtgttttagagacctaccaaaagtttcaggcagcaagcaaaattgtttagtaaaaggagtgagtgagtgttgaggggaagagtttggtgagataagatgccgggaagtgatagggggaaggagacaggctgggaagtgaaacattataaagtattgaaggctgggcagctgagttagattcgatgcaaaaggatgtatgatggcatgaaaaaacgcagctgtgagttctgatctaaagtttgttttgacccatatagagcagactcatttaacatttaattagtttatactgtgtgtcagttgtggtggcaggtagacatttgcatagatctatattttaaattttgatcctcatgacacctctctgggttagataagtattattctctttgttaagctgaaggaggctcaccaaggttgaatgacttgcttaaggacacaaaagcagcacacagaactgtgatgcaaagtgaggtctttctactgcatcactgcagggcaaagaaattaaaatgtcacctttctttagtattgacttgttattctttatcttctccttaaatacacagtctgaaagcaaggttaatatgatgctttttctcatattaattaaaatgatgtttatataactaatccttgcatagtacaacttcataagtgaataaaaaagagacagatctttaactgaggtatttattataaaatttcgtggcagagacagttacttctaaccaaatacagaatgctctccactatgtatttcccagtttttcctgctgttaggtgaggagcagctgaggcaataaagaattggtgtgcttcctccatatctttcttgacctgctgtggtgtccttggaagacacatgcaccagatggaagaagacaattcatgcctcagaattcacatgagcgaaaccatgtcttgatggtattgcactacaagatatccagtgtctatttgttacagcagcatagcccaacctatcctgattaatacagatccttctgactttaattgatacaaggaaagtgaccatatctgtcttgcacatcctcttatccctactgctgaatacaggacctcccacataatatatgctcaataaagtttgattagtggaaaatgaatgaatctcaaattgagtctggcctctgtctactatttggttaaaccacttatttactgcttttagaaaccagtggttgatcaatctttttgatatatcatttcatcagatcactcctcagattaagactttattatatatttaatcaattttaaactccttcctttgacattcaaggtccccataatctcacctagattcctacaactttaccaatttattgctctaatccaatctaacctatcactactggcctgtgacccctggtcactatattcactgttctagggacaaacatttagtgttcttaagcaaatcatttcaagtctctgagcctcagtttctcattctgtaaagttgttatttgtagcaaaagacataatgcatacaaaagtttaagttatgcagcattatgaaaatccaaatttattttgtacttgttgttggtatatattaaataaattgatagatcagaggagtgacattaaaaaatgtgttgccatttcagtagcacatttgtcaaaatctcccatgacaatcttgcatggaattgctgtctagggagaaatggtaaaggaaaccagggagaccctgttggttatctactccaaattcattccctcttttttttcttaactctacctctattttatttaagttctgcccttctccttgggaccatatttatctagggaggatcctttacaatggccctattttcactgccaatgattactgtagtcatgaactctgatgcgtttctggttgataagttgtggaaaagagtgtaagagggcttctaagaagggtttctgtgcttttaaaaagaaacactcagaaaatatataataatttcttcttctggacagtgtcatgtctcgaaatgttgcagccagctcatcagcatgaaaggatctctagcccgaggtcagagctgacacgctgagatgagaatggaggggcagaaacacggaaagaacatagatccttgatgatgtcaggagtcactgattaactaaccctggagctccttctgtcctgatttctagttatgtgaaataactcataatcttatcttttgatctaattgagtcctagttttctgttacgtgcaatggaaagcagtgaaaccctcttagactgagtccattagggtacccgtaacaaaatactataggctgggtggctttcataacagaaattcatattctcatggttatgaaggctggaagtcccagatcaaggtgcagcaggtttagattctcttgaagcctctctccttggcttgcggatggccaccttcttgctgtgtcctcacatggccctccctctgtgagtgcacatctctggtgtctcttcctctctcataaagtcacaaggcagatttgattagggcccaccctaagggcctcattttaacctaaccacctctttaaaggctctgtctccaaatacagtcacattctgaggtactgggggttagagctttgaaatatgaattttttttttcttttattttttttttgagatggagtctcactctgtcgcctaggctggagtccagcagtgtgatcttggctcactgcaacctccgcctcctgtgtttaagtgattctcctgcctcagcctcccgagtagctgggattacaggcacatgccaccacacccggctaatttttgtatttttagtagagatgggtttcaccatgttggtcaggctggtctcgaactcttgacctcaagtgatccgcccacctcggcctcccaaagtgctgggattacaggcgtgagccaccgcacctggcccaaatatgaattttgtgggggacacagttcagcctataacatttgctcaagccaaaaaaaggagtttaaaatttcagaaaaaagcctcacagactccaagtgcactttatcaaagtcagacatccagagaactgggaagccaataggcagctaatctcttcatgtttttccctcttcatggcttcaattttacatttctctcagcatgcatgctttggttttttctttctcctcacccttctgcttatacaaagctcatcatgagtgcccgatgatgggcttcagcccctaagtcacatgacccttatgtttggctacccctagctagcaaactcaggctccatgcctctttgtgaaattcttgagatagagaatctgattggcccagtttgtatcaagtgtttatccttgatccaataggctgtggccaggggcatgggtcaggcggtacacaagggctgcctcttgtgatctgtggctaagttagctcacccagaggacttgaatgggaataaattccttagcagctctagtggaagagggtaatgtggataaaattatagtgtagtcaagtggatttgaagcagatgaaacagtcatacacaaaggtgttgattcattactgacaggctgtaagaagtctttagtaaagtcccactttatcttgccctcggttctgattttttttttaatgttcccaatattttaataaacagattaggacatagaaaatatacctacgaatttttagttgacaaaatgatggaaacatttgttaagatagttagaaatgagattcagactcaaaatcatcttaaagggctagaaatctgtgtagaatcaataagataaaaattagctcacataaatattaacttccaaatttggatttcagtgctcacttgcaaagtgatggaatggagttttcaaaatgaactcttaaataaattatagcactctgtggatatgtgtctctctttccaagaaggaagtcctttttaattagtcgcatgctaaagttagtttggctaagtaaatgttttcctagggttgggagatttctataatttcagtttcccttttgtttgcagatcaagtagtttaaatgacccagtaaataatttggagtcaccacagttaactcaccaaccttgaaaatccctgaaccaagaggccccttgatactgtaagggagggctaccataattaatagcataagtctaaccagggtaccatacctttccctatattgctggatattctgtaagtttggctttcccaaattaggacacagagataaggataaactcactttccagctatttcgttatgccttttttatttctttcaacttttggaaatgaccttatgaatttcctcttattatgaaatatatctttattgaaaactggagtccaaacattacaaaaaatacatagcataggaagtgaaatgaaatactgcataaccaatgtaaatgattaggtgactcctctttcagattacattcctgagcattttctaacaaattatcaatatttttattttaatgaatatagaatcacaccctacttattcttttgtaatttgcttttcccatgtaataatatggcctgcaggctgtaatgatagacttgttttttgcagaacaatccattagccaagggcaactagaaaagccagacaaaatattcgattgatttggtttgaaggcgtgagagagctaccatgtctgcaaggacttgaggggctaagatttttgagaaaagggaagtacaaggagtgtgctcgacattcagtctgctttcttctcaaggtctttagctgattcacaagtagtggctgagagcagagaagctgagctaagcttctggtagtcttatcatactgaagggaaaaattagaattcagagccctaaaaggaaaagagtccgtgataagcatcccaaggtttttgttgacgtccaccaagggctataccctagaacataggagtgaaccagaaatagaccaaccctcacaaagaacaaagtctagctttgattaaacattatccgtgattggattaggtgatctgcctctatgctaacagtttactaaaaataaaaatgaatcttccttgaaagaaaataaaattaccaagaggctcaaattatcttaatcttatatacacatctggcatctaatttcaaactaccagtcatttaaggagacaagaccacatgactgaagaaatagatgagaggggcggaccctaagagtgtcgggatattggagttgctagacattgactttaaaataactgtgattacatggtcattctccactcacattactcaagagatacttagtacatggacttagcacatgagctgtgtatgtaatagtccctcaacaaatatttaatgatatgaactgataatccaaaataatggtactaatatatttgcaattttaacctaagtaatatatgggcaattggggtttctgaacaccctattaagtcttactgtctgagggctccattaaatcacctcaggttccttgagatgatttaatggagcccacagacactaaattacatggagaaagtcactcttttctattttttttaagagaaggttctcactctgttgtccaggctgtagtgcagtagtgcaatcatagctcactgcagcctcaaactcctgggctcaagtgatcctcctgcctcagcctcttgagtagctgggactacaggcatgcatcatcatatctaactaagtttctaacaaaaattttgtaaagacagggtctcactatgttgcccaagctagtcttaaactcctggcctcaagtgatcctcttacctcccaaagtgctagaattataggtgtgagatacctggccccttttctgtttccttttagtccttctgattatgccatagagggagtctcagcttggtgctgacgtgtcttttataccatcctaatgctatttgattttttttttttttaacagagaaagatctcaagcttctcagtcaatgtcttggtatctctactctggtgttcttaaggaaaatgctatgggctcctttggacatctggtaaagtctatgaaccctatatcaaaataatgcttgtaaattcttacaataaaatacctagtattacaaagtaaagaatatgttgaaatacagttccatccaccatccttttgccactacagtttgtgattgttgtatttattttcatggttactttctacttatggaaagtgcaattggtttctgtttatagtcatgacatacaatttgcattttaaatacatttgtttaaattaaagagggaagatgaattaaggaaataagtaaaagtaggaaatatgacaaagctaaaagtggtgtgctgtgattgagtgacgaatgcagacttaacccaagagatgactttattaatgggaatttcaggcaccctttcacattggggactgtactaaaccaggcttgacacaagcctcacttgtccatcagtcaatgtccattgattgagcagccccaccaagtaccagacactgttccaggtgtgagggcagggcattgagcaaggcaatttattctcaaggtgtttacattgtagggaaaggatatagacaagaggcaggccaaaacattcaattaaaaggtaacttcagatactgatttttgtcctgaagggcaaaagggtgactagcagggtgactagagagggagtgacttggggtaggtgtaggacagtagcagtggggtttgtggcttggcccagggctgcattagaaaaagaccaggctcccactgcagcagtgcccggaaaaatgcagagacgagccaggatttctggatcattaccatgaggaagggaataaatgtgtgttctctttcagttgctcctcttgacacctgggtaaatggctgtttacacaggcagatcacccagccgctccctcaaaggtaaacagctgctttctgcctcccctgctagcacagattggggccctgaaacagtgtgtacccaggcaaaagaggaggtattctccatttaggtttccattcattcattcaggcaagatttcttgagtgcctcctaaaatgcaggcaccaaggtaggtactgggaacacagtaaggaacaataaagaggtccctcctcccccacctctctctcctctccacttagaatccaagctagaggtgtgcctcagagtctcaggccactcctctcatgatacagatggaaatatcagaatcagaagaggcaaagtgacttgcccaagacccggagctagcccacttcaagcaagtcaggtctcaaaacggtgccagctgctttcattgcctactacctgacttgctcaatcttggtcagtatttggattggcactttttagtgtattctccattactagactattatggcttagggaaggagaaaccagaggtccatcctacaagcacactgaaatgtaatccaattctaagtccccgtattacaacatggactattgtgactgaccacatttgccttatatttaccttcttccgcagggatatgcccgatttcagtttcccatgttgaaatagcctacagttgagacaaacatacattacatttaagcataccttccattttttataaggtactatatttttaattgtttcaatcactgtgaaattaacaaatgttcatgagcacccactgtgggagctcacagtgtgggagttccgccctggtggaggtactgggatttaatgataaaccaaagacaaatatatgctcaatctggtctctgccctcatatctagtctgggaggtaagcatttgtcaaataatcgcacatatatgattgtgtatgtatgtataaaactaaacgctgtcagttgtttaatttaaaaaggtcaaaaagaagtttgctttgaagaatgagatcaaggaataaaaattagtcagttaaaaaaaattacatatataaacataaatttaccaccataatagatacttgttttagtgcaggtcctccgtgatgcacactaagacaggaataaacaggtaagggatttattggggaaaatgcctgtgagtgaaaattggaggtcaagcacgatggctcatgcatataatcccagcactttgggaggccgaggtaggtaggctgcttgagcccaggagttcaaaatgagcctgggaaacacagcaaaaccccatctctacaaaaaaacatacaaaaattagccagctatggtgccgcatgcctgtcatcccacctactgggaggctgaggtgggagaatcacttgagccagggagttccaggctacagtgagtcttgatcacgccactgtgctccagcctgggcaacagaccgagaccgtgtctcaaaaaaataaataaataaaataaaataaatgaaaagaaaaagtaaaagaaaatggtgggggaggctggagaaggccaggaaagccatcagactgggaagtagttctgtgaagaggagagaggaggatggatggtcttagattcagtgtcatcctaaggaagttctggcaaggccacatttgcccatcagaagagtccccctttctcaaggatggaactggcttagttttcctgctgggcttagctacaagctgggaacagccattgggaagtgtggtctctgcagacatgtggtgatggatttggagtacagcagctggggctgtccatcagtaagggatctaagaattacatgctcatggccactgcaatgctaaaatagagcaaaacacaataaaaacaagataaattagagggacatgatctggtctgggacaaggaagattgtttcagaaaatagtctctgtcttacattcacaagggaaaagagcaatgttccagaaagagtggcgagtgcaaaggccctgtggcagaaggcagcatgggatgttcaagaaactgaaagaaggccagagcagcctaatggaaataatataggagagagtggaatgggaggctgagaggtgagcattgctggggacccctacaaggcctttaaggagaggacattgttttaaacacacatctaaaactcactacttacatgacagacatagggcagagggtggagtactaggcgtgtgcctgggtttccaggtggctgaactgtagcatgactggcgggactgaaggagtggagaggctaaggtcttgatgtcacctacttttgttaggattagaacaggggtcacaaactgttggaccccatggcaaatgtaattttctttttcaattttttattttttgagacagtctcactctgtcacacaggctagagtgcagtggcatgatctcagctcgcggcagcctcaaactccctggctcaagccatgctcccccacctcagcctcctgagtagctgggactacaggcacatgccactccacccggctaattttttttctttttcttttttgtagagatggggtctcaccttgttgttcaggttgatctcgaattcttgggctcaagcagtcctcccatctaggcttcccaaaatgctgggattacagacatgagccactgcacccagccacaaagataacctaaagatgtgtttactttgacccaggcagtagtttaaaaaagttttaatttgttgttcacatttaaaaactggacaatttctacataaaaatctgaattactcatgtctcttaaaaaaataacatctagcaatggtaggcccacattccttcctgaaaataattagctgggaaagagtagggactgacccctttagacacggtataaatagcatgggagttgatcagtaaatatttgctgaatgaaagaatacatgaatgaaaagtcagagccctataggtcagcatggacggcggtaaaggaacctggctgagcctgaaagagaatgtgatctaagattaaatccaggatatgctggtaaatgtttaacagccaactctttggggaggaaaaaagtcccaatttgtagtgtttgccgattattgtgatgtaaatactcccatcatgaccaatttcaagctaccaacatgctgacactgaacttggagttggaaggagatgaacaggcataatcaggtctcgtgagatggcccaagccggccccagcactccactgttatatatgaggctagaattactacataactggaatagcaactttctggaccatatgcctggaacacagcaggtgctgaataaatgtttgttgatccaggaactgactgtgttgaagcccacagatgggaaatcagtagaaggcaggtaagagtaaaaagaagggcagagaattgggggtacagacccctgaaccataagtcagaggaatgttgtacatgttttcagatccctcactggtcaaatgaaggcaaagggttagatctctccaaatctttagagggacatgatgtaactccattaagtaactcagtgattttcaacattaaaaagtgtaattatcttttcaaactaaatattacaaataaattctgataaaaggaaatgtacgtctgttgagacccctgatcctataactggtttagtattccctgaagccacttgtagtagcctattcacatggggactccccagtgaacatttcctcatatccacactcttgtgaaggtccttcaccttgaatctggactgggcctatggcctgatttgatgcatagaatgcacagatgagaataggtgggtttctggcataagctcaaagaaggcacagtggcttccagagccctgagctactagtaagaaagtccagctaccctgctgggaaggccatggagagaggccatggggagctcatagagaaagagagggaccctgagactacatggagctaggaagaacaacacccagccatcccagtgtcccagctgagccccactccaaacccatctgccaactgagcacatctacaagagtaaccactgacaggccagcacaagcccagccaagtccagttcaaattgcagattgtaagcaaataaagcagttgatgttttaaggtttttagatgatcttagagtttagattatgttaagtatgtgagatccctaaatagccatcaataaaaaaattgatcagtacaatggagaactgcacaactattaaaagaagaaggcaaatccatgtttaccgacatgaaagaaaatcaccaagatttaccctaacatgacaaagcaagattcagaagaatctgtagggtatagtacgggatttgccattgcatgaaaaagggaaggtaagtatttgtgcatgcgtgcatgtgcatttgtgtgtgtgtgtgtgtgtgtgtgactataaatttgtaaatgcatagaagatttctggaaagatacataagacattagaacagactggaagacttgaagaagaggggagattgtgtatccttttgtacatattttcattgtgtatccttttgtacatatttttaccttatttgcaggtatcacttattcaaatcatagaaataacattttaacaaaaaagtgaggcctgcttatataatgttgaagacaactagctaccacactatgggaacacactattatattaaacttgatgattcgagttttgcctaaattttactcagaatcgaatacaagaatagaggaaagaatttaaaacccatctggctaatgcatacccttgccaactattctattagaaaggaagatggcagtggggagggggttcttacaatggaaaaggtattggcttccctagtgcctgaggtcgggttctccaagaagcaaattcagagtgaaggattttaatgagagtaatctatgtgggaagtggtcccaagaagcaccaaaacagaagtggggaaaggacacagagaaggaaaggaagtcactgaagggactgtgagttacctttgtgggcaactggggtttcgtcccactggggacctctgggagacactgtaggacatgtttcagggtgtcccatcctaagtgggaggaatctggagaatttatactctgctcccaggggcatcaattctccagtgtttccagtcagctctgcttgctggctgagataccagagcaagtgctggtctggggaagttgtcagcgtgtagaggaggtgagtactgagcagatacaggcagggcactgtcagtgcatttgcttacctagacagcaaagccagagacaaaagctcgtgtgcttatgtggcaggagagagagagcgaaccatgcagggaagtgcattactttatgattttttttttttttgagatggagttttgctcttgttgaccaggctggagtgcagtggtgctatatcagttcactgcaacgtctccctcccagtttcaagcaattctcctgccctcagcctcccaggtagctgggattacaggcgctcatcaccaagcctggctattttttttttttttgtattttttagtggagatggagttttgccatgttggccaggctggtctcaaactcctgacctcaggtgatccacctgcctcagcctcccaaagtgctgggattacaggcgtgagccaccgtgcccagccaacaagttgattttttagttttatcagatgttttctaggagaccataggaagctactttgtctacacattacgcctctgtaaaagaggaaggaagaaatttccaggggacttcggctcctctgcacttctgcattgtccatgtaaaggtgcttattgagtatggattgtggtgtcttgcaccttggtggcagcaggaaagtcccggggcaggaagtgagtggtacataatgcaggagaggggagaggaggtgcctgataaccctgagagtctgggagctgctgccccagtgagctcctccatctgtaacactggcggaaactgtagtaagcaggctttacaacagtgggaaacatgcatgccgatgtcagggcctcccatccaaaagcaaggccaggatggatccagggtggtgaataaatgaaacctaatccagaacctaacaaaggagactgcgccaaggctaagaggagtgccctgacagttcacagcatcttagagcacattgcaccacattatgctatttcaccaaggagaaaattcataaacatttttaaaagtagattctcaagactagacattattttaatattgcttattgatgattataaggtaaagaatgttggtgagcaggtagaaaagaaaggtaattactaggacctaattctggttctccagaaatagatcatagtgtaccaacaaatttccatttttgatagaattactttgctgatatacattgatctaagcattgcttctggccatgtcactgataatatttttctggacaacatgccaaaacacaggtggaatgactgtacagtcaaaaatattttttgactatacagcagatttcaatcaactatactcagctctctagagatatgccacaggtgcttgtttttactcttgtgtttattgtattcgatattctttcctcaatgatttggaagaagatgtagcaggcatactcaccacatttatggaaggaatcattggcatgtttgatggcagaatcaggattcaaattgtactgacaggtcaggatgttaggtcaacaccaaccatatgcaatttaagagtaatagaatctgtatttaacattaaaaagtcagttgtgttggcacaggattggggagttggattgacagcaattgagttggaaaagacctgtgggttttaattaacccaaagttcaataagatccaacaatgaaacagcttattttttaaagcaattttaggctgcagtaataaaagccttattctcagctgaagagcagtgatgatcctaatagattctgtgaggctacatctggagtattgtgtactcagttctgggtgaaagagtgagcaattacatacaggagggatttcaaatgcatgtcacacaaggactggttatcagcaccggggataaatgcgaacaaaatgataatattttacaggaataatagtgacctccatctatttgatgagtattcaggtgtaaaagggagtaaacctattctttttgctctatgctttagtcagctatggtcacattaataccatgttggcttgtaataacatctatttatctcttatgcattcacagataagctggagtttgtctgatctaatgtaggcttggctgggcttggctccaggccatgattgtggttcaggtttgctgcatgtgtttctttttctttgggaatcagtaggtgcccctgggaatatttttcttacagtgatagaagaggcacacgagggtgcatgcattaaaagcctctgctgatatcatgtctgccaatatccattgcccaagttaagccatcccaaatttaaggggcttcagccactgtgaggccatgggaagggaactgcatgtattcctattatggagtagtggaacaaataatttaatttattccactccagactagcaggtcaaaatgagggtcagtggtctgaagctataggaagatagattttggtaataaaaaactggatgatcgttggaactgtccaaagatggatgcaaacaactgaagtctttgtcccagaagggattagacagagttcaatgggtcatttcccaagagcatcccagatggaattccggtgtcatttggaaagttagagactgagccagttagtatctttcctactccaacatcctcaaattccatagtcctgttctatcccatactcttcctcttgactctgacatcctgaggcttttactctctgtgccattccttggggaacctaattatatatggcttctttgtgtctctaagagattccatgagtatttcttgtcttcctaattgctttggaagcatttcagtgtgcaggctgagtcttaagtttctcttaaattgcctatagctattagctcagtgtcaggcaccaagcccttagcatggcattaaatcatcacccaacacccagtgcagcctcaaaccatctttgcagcctctctcctacctttcccatcttattatccagccgaattgagatacctgtccttcccgatacacagcccctgcttttcaagtctttgctcacttatagaccattatcctcccttgtctagaccccatccatcccactcagtgcaaaccaagtgctaccttttccaagatgttttcactgatgctccaaccaaaaagaagtttctctacctcttctgggtggtcttaacacttctcatggtattcgagatgtttgtggtatagaaaagtgattctccactctaactatccatgatgacttgagaatacttaaatcatacacgcccaggtcccatcccaagatagtctcatattattgatctggaaacagaaactccaagtaatagcagtgtcagcaataaagcaggaactcacatagcaaagtgtatggaaataagttattccagggttggtatggcagtttaatgatgccaccaagaacctaggctctttcttcctgatctgccattcccaatatatcagcaatgtctttcctcatggttgcaaaacagctgctatggctccaagcatcacatcctcacaccagcataccaggaaggaacaaagcaagcataggaaaagtgttcttctcacatatctctcttgctcgagaaagagaaccaagaagtttcctgcaaggaacaaagcaagcataggaaaagtgttcttctcacatatctctcttgctcgagaaagagaaccaggaagtttcctgcagacatttcctcacacctcattggctggaactgggtcacatgcacatccttgaactatcattggcaaagggaaatgggtcatcaaaattgcttaaggccaagcaggagccattgttggggttaaactgtcctgaacaaaatctaggctctgaatgataggcaagaaagaagaggggatggccacaacttcctttacacggtgaataacaagagcagcttaagtaagtagatgcccctatggggcagtaatcttgctgtgggcagaaacatgtccatgcacacagaagctttaggaatcttcagagaggttccatagtcacttgtgaaccactgaagcctctttctccaagccattgtcttgaatgctgtttttaactgattacaatgcatgcagctcccgtgccccacttattttagcttttgaggatgttcatttccccggaatctccaaatataaatcaggttttcacagaaaaccacagagccctcacaggtgtggaaatcatctaaagatatgctaatgatttgggtttctagaaaaatgtatatctatgaaggaggcagggattaagcacagctagtatgtatgattttttcttttcctttcatcacctgtaataaggcaggatggagactgaatgtatcagacaaaacagggatcaaatactagtttcaacatttgctgcctgtttgaccttgggcaaattctttaacctctctgagccttggtgccttgttctcctcatgcataaaatgaaccctcctgcagggatgatatgtagaccaacactgataataatgacagtaataccagttaccctgtcatgggtgtccattccatgacaggcctgactctaggcattttatacacattctcttcttttttttttttttgagacatggtcttgctttgttgcttaggctggagtggagtggtgcaattgcagctcactgtagcctcaaactcctgggctcaagtgatcctaccatctcagcctcctgtctgaatagctgggactacaaatttcagctaattttttttttcatttttttgtagaggctgaggttttgctatattgcccaggctggttctcaaacttctagcctcaagtgatcctcctgctttggcctcccaaattacaggcatgagccactgcacctggccacgtcatcgtcacaaaaaatgcaatgagttaggggcttgtgttgctcccaacgtttagagtattgaagggaccagcccaaagtcacaaagctaatgggcaagggttgaatccatatgtctgactccaaagccagttctgcagcatattaattgcatatttcttggtttctttttttttcctccaattatttggtgctatgccaaaatcatcagaattgttcctgtaggcattccttttcttaattacatattgcattgttctctttctcactttcccttgtttgatccaaggtaaatttttagtctttttaaaaatgctcatacattataaatccttgctaatatacagttctttcagactgtcctgccttgcttgaggaagcagatgtgggtcagggagaaagaaggaccgatgaactcaaatagccttttcacttttattctctcttttcacttcaaaatgctgtgtcatcagtgaatcccctcctcataatctcttctttcctggagaataccagcctagtttcacttcccttgtaaaatatccacagatctttgtgccccaatcactttggctgtattgcactgggccttgctaatggcacaatatccttttcataataaggtatcccacatgacacagtagacttttgtacctggccagaatcactacattcatcgcagataccatgtgctgtctattggataccctcattttctcttttcaggaagggctggtgtcagctttcctcctgcctcttatgaaccaggaaatttttctccctgtatcttggggctgctgttttccagcctctgccaagtttccatttttgtgggacatcagttgaccggcatacccgtaccctggacggggacttgggttaactgagggttggcattctccccaaagcctgttctgcatatatttttgaaaacctattggttttctgcctaccttctttcctccatgagtagacttcaatgaacacatcactctctttcttcggtaattacctggtctcacctgctcatcataaaagagctattcttattgttagggctgacagattcactttgcaggtgatttaatctgcatctgtagtgtccacagaacatattctagaagttaaaagtacataatataatgaaaaccctgatgcaacttttgcttgaaatatgagtatagcaaagacatttgagagaagagcctgaaaataggttaatagtagtttaattgtatctaataagagctaccattgattaagcctataataatactagtaactcccctaaatatgctctacacattacttcatttaatctacataataacactcagaggaatgtattaccatcccagatttacagagtaaatgtattaccatcccagattcacagagaaaattaaggctaagaatgttaggtaattggccgagtagcctgcagctagtaagtatcagagaacggatttgaacccaggccttggctggtttccaagtccacaagcttagctgctgctacccagcttcactgatttttgttccgtgcactgcctctaggatgtttccgtatttgccttctggttaatatttactccctcctccctagcacaccacattaagggtgtggcatgggcttttcacttcctgaatgtttgttactcagtttcaaccagctttactctctttcaaagtgacataaaagtgacagccctttaagctcacagaaaaacaggagctgattccagtacgcgattgactaccatgcaagattttgtctttccttttatattgttgaagattccttgatggcaacatggcccacacattatctaacctccagacaatcccagaggatctgcaggaaaccaccaagcaccccagaggggtcactggcaacaaatggattaaggaaatggcctgatttatgggattattgggggtcggggtggagaattatagacaattttgtttctactgcagaatcagcatgatagagtgaatatggactttggagtcaggcccaggcctgtaggatgacctgggaagcagtctaggcagctttggcccacttcagcttgtaagatagagataatacatcatcctagaaatatcgcatggattatcagattttgtatataaagtccccagcagacacctggcacacagtaagtgctcccaaaaccaaagcaacagttcttagaagcttagcttggctcaggctgaagtctcaaatgatctagggttcactctccagccatttctaagccattgagtgaaactggacatgttagttctggtgttgacttcagtatcctaagttgggaaatagaactcatcattccactcctccttttaagtattttgataagaataaccgagaagtccatgtagcattttgaaaatataaaatactattagattggttttgttttgttttcataggaaagagaaaggaagaaggaagggagaggagaaaaagagaaaagaattcaggactgatattttcaaatttccaaagtgagcaagattggtgtttggtactctttgatctctccttttcttaaaaaaaagggggatgagtgtgttagagtcctaccccatctagaattagagagagagggaaacagagaggtaaaaatatccttcctattatccctcaaattttgccacagagaaaacaactcactctgcttttgtatcaatgaccttactgcctcagtgtcctcatctgtcgaaatgtgtttaggaatagttgccctgcacacctgtccaaggtgttagaagagccagatagataagacaatggggaaacttattttgaaaatcaagtagtattaaaatcaaggtggagagaaggaacaccaggccaagagtgagcaaatctgcatttgaacctgggcttggatgatcagcagctttgtgactttgggcaagtcatttcacctctccacactgcaggttcctcatcttgcaatgtggaaagcagaacctctccgggttggaggatcctgtgggatcccacgtgagtcagcaccttgaaattgcacatacagggcacatcatttctaccatcacctaaggtcaatcccaggttattgctggccttttggtttgctatgttttccacctttctcacaaattaaaaatgcagacatgtaaattctctatctatgtgcacttcaggaactttcaagatccctttagggttatatctttccttagcctcttgcaaacctgaaagcaaggtgactgtaggacaaactgtgtgggcactggatggggaatgcatgcctctggtagaaggggaagccagaattcagagccaaagaacaggcaccatgcaggactatgagtccaggatgtccaaatctgacttctaaaggaatgccagtgatactgattgtttttaatataaaatctaccatttacaaaatacaggctcaagaacaattgtaaacactgcacagggcaagttttattgtttttcttaaaagagctgaaaaccataatcaagtgcatctgccctttgttgtagattggggcctgcaatctagggagtagagaaaggctgcctgactggttagcaattgaaacacagagctggggccatctagtccgaatgcagtgttcatcgttctttgctaaacacaaggtgaccgtggttgttgtggttagcaaggtcacctagcttcctaggcagaccttattccaccacaatgggcttcctttgctagaaaggacatggaaaaaggagtgacaaaagaggagaaagagctagtgaaggaaagagaggaagagagaggaaggtccccagttgttattacctagggagtgcccagcatgcctttcagaggaacacaggtgcctacccctttctcctgcctgctacagcccaccaccaccaataacaactgtgcaccctcactgttgcccccatgcatggagcagagatagagcacagatagggagaaaagaattttcaagggtgagttggaatagtgacttctgcaagcctggtaggtttgtctcttttccggtatccttgccactgcaccaggtgaaggggaagcagactgtaaaatactttgaatattatcaaggttatttggggaaaaaaaacagcaacgacctcacctgattgtgccaggtttgtgataggaatctggggggaagttgtcctcaatgacctccatttcttgaatgacccgtggcccacctgctacctctgagatgagtcaggggacacaactttcttgatttccatcatcatttgcctgctactgctgcagagactcaagtggaaaatgtcacttcttgaagtcatggctttggtgtctggtgacagggagtagatagagtttggacagagccacttgtgagctgcgtggtttaatctctctgaacttcagtgtctctagctctaaaatgggggtagcagcctcaatgagcaaattaaagcaaaataacaaatgtaaagcacttatcccaagacaaggcacatgagaagtattcaacaaaggacagctctagttatggtggtgatggtctctaaagcttccagactcagattcctaaccagacagccatgagacatggtccccacagtggcaaaagggtaggctccaggctgcctaagtttaaatatcctgactctaccacttcagcaaggtactaaactctgtgcttcttgcctcatctgtaagatggagatacctcattgggctgttgggaagattgacaggggtcacccttataatgtgctcagcacaatgcttggaccaggatggatgctgcttgggtgatgttatcatccaggtgggcactgtggaacacagactgatacttaagttcagcacttgcctcatggagcttacagtccagatagaataggtgcacatgagatttttctcagcaaagttgtagtcagtgctgcccgtgccacccatctggtctcagcattgctgctcatgtgcactgtcgtgacccatctttctgctgctgaccttaagaaaccttgccttaaggtatgctttggggaaaccaaattatgacagatataaactgaagtgccaagacgaatctttcaaagtgctgtgatgtccagatacgtggcaagtgccaggaatttgtgtttgggcccactgggagagaggtccttttccccggcttcccgcagtggaaagctgctgtatgtctgatacctgtgctccaaatctaccctcgagttcttatttggcaacaggcttgcttagcaatgtccctggggtcagggagccacgggtgggctgatgcaagaaactgcagttgtgtctctaggctgcgacagcatcacagctgtgagcttctcaatttaaactcaacatctgggcacaagaggaagcaccctccatccaggcaagttgaagttttctttcctgtatctttaacagaggttaaaatgtaaacacaaaacaaagcaaaacccaacttaactctctgggtcctgtttctgttaaaaacattttttaaaaaataataataattagaacaatagacccagctgagagaagtctgtctcataataacaagagagactaaaacacgctctcccacaaacagataaggaaatagtgagctaaaaagtaaaacttaagagtgctacttctcaactcagtaattgattcatgaataagctctagcccaattttctatgaaaaataacaacatagagaaaagaaaagccactcagatgcagctatgtcacccaagtgttcactggagaaatttaatatgtgtcagagaaaaatctgccttctccaaaacattgccccctccaaaaaaaatgcttctcgttgtgggattttttcctccacactgacagagagtttgaaagtaaagcagccgactagagtgtgggtgcccacgccttagaacaaagagattaaagaataggcttttcttcaaataaattcgatttttttgcaacaaattcagggcctggacagtttaatgaagaataaaaaatgctcagacccctctctttcagcttttccaaacaaacacagtcatttccaaccactcacaggtgtttttcttcccaaaacaaacacagaaacaaatatttacagagttatgaaaattcaatagaagtcacaggggcacaaatttcctcacctgcatccaagatctctggcctgggattatcaaacagaacgtcacacctactttccattacgggacaggaaatctttgagccaaagagaatcagtctgcagaatgtgttcgtaaggataaaggcctcatcttttcaagaaagtgtttgcagctccaaggaagaatggactttggacaaaggaaacaccagtgactttttcctcatctgcccatacgtgcagaaatgcccttgaagagctcatgactgccttgcgaattaatccctatgttgaccaacaacacaaagagtggtgctgactaccacactgaccaccaggaataatttcctacttaggtgtttgcgggaaaggctgttgtttgcttttctcaaatgtcccggatgtgaacaggtctcaaaattaggaaatgcagctgggaatggctgctatcaacaacccttatttcatcttttttctttcttcaatagagctagagaagcaggcaagaaaagaaaataatgcaattatgaaaggagggaggaaagacagagggaaggcagcggggcagaagggaagcggaaaaaagatgggagagaaaggaagaaagggagggcagaatggggacaaaagatggcagggagggagcgagggaggaaggagcagaaaggagagggaagaagaaaggaaggaaggagagaggcagagagagagacaaaaagaaagaatgagagagaaacctgctgttgtcagaacctgactggtttacgctgaggaaccagatagcctctcagtgaaaagctgagattaagctcacaacataatttccattatgttaatgttgctttaaaatatattgcacagagtgctgcaacaatcaaattatcaattataatgtctactcagagtagaaaagtgttgcaaacagagggctgattactgcttcagtacatccaatctgcatactcattgttcagtacagggaaaacaaattatcttgtgttggccccagttcaaatttaattaaatgagtgcaaagcaattataataaagtacaatagtgtagtaatgaaaggaaaataactcattaacctattaatctttatttatgttcctctcaaataactatgtaatttctttatcaccgcatcccataagaaaaagtagggtctcgctgctcatggcctggtttaacaggttctatttggttaagatgcctttgtgggcaccatttatactcactttgcttagctttggggccacgcaccccccagctgtctgtcatgctccctgcccacacgtatgcgtacctgatctagatttagcgctcctctcccacttttccctgacacttgtcagttacagaaggtaaatctctctggaagtgagaagtgcttaccacagcatatgggaaagaggcatcttccagcatctggtatgtaaggcaatgcaaaaaaggggagaggacagaatcacttctggtttagagagagcccagtgcttgacagctgaagagattccactaaaagctaacactccactgtgctttccagttaataaagctttttacatgtatgatttcactgaggcctcctgataacactgtgagggtgcctgatgtacagatgaggaaacagaatgatggagtagaaaagtatggtgtccaagccttgctactcatgagtagcagatccaggattctaatttaggtctacagcccctaaatcttcatttcattcccctagaaccttgctactcaaagtgtggtccagggaccaggagcattggcgccacctggaagctcattggaatcttgggcctcaccctagacctgcagaataagactccacattttataagatcctctagtgattctcacacactgaagtttaagaagcactggaactattggatagtacactgccttttgagagaatggtgactcaggcaaatctcccagcaactacttggttaagcatttccttgcaatttgcaacaccttgagatgaccttgagatgatcttgggtgatcaaggttgattctggcttccattttcatgttatctgaagacagggctggaagtagaggacatagtaaaagcagaaatgatttctctttctggatccacagcagacccgaaaatcttcccctttttcttccaaagagaatttgtgaaatagagagaaaggagaggggagaaactgatgaattaaggaaggaaacagacactcgttaagtttctaccctgttcttggaggttttccatgtattccttcatttaatcttcacaacaagcacctttttaagataaggtgactgacgacaggtttaagtgatatgcccaaggtcacccacactgctgggtaacagaaatggtatttggcttggattcaaagtttacactttcactgtactctattttctcttaaaaaaaaaaaaggaaaagggaaggaaatcaagatgaaataaaagggtggtgtggtagtcattagtgctgttcaccaagaatctccagtttttagtctctgaggtatgtgaaggattatacttcctgccctgcttagaagtaggtgggagccatgtaaataggtttggccaatgaattggcagcagcggtttcatgtttcattttcagatcaaatatttaattgcttgtggacaccttacagagctatctctctttcctctgccatgaaaattgccaatattcaagatggtggttgcatcataaacctgtatccatagtgagtgaatatgctcaccagagacccccaccaacctatgatagacacataatgagagtgataaaagcaataaacctttgtgttattttgagcccctgaagtgtgatggttgcttgctactgtagcatcactgagcccatccccactcttccagatgctaggaagacgtgtgaagggaaacagttaacatttctacactgcaactttttcacttgagactcacagcattcctatgagaaaggtaaggaatggctattatttattatgtatttataaattactatttacagataatttaacactgacatagcagtttaagtgtcctgcccaagatcacaaagtttataagtggaagggctaggtttggagcacaaggcttgtaatgactgatctttcttcaatgcagaacacttcctccaaaaaaggactggagcaccattacctgcccttgggtatactcagattggtatatctagctttgtactcacactcctcttcccccaccactcaggaggaatccaagttattgggcaaaaatcaggccaaggaaaaacaagtacatgcagttgccagcagatgacgacattgggtctttgctagagaaaccaccagagaaagttcacagtgataaccctcaatgaagtcaactgtaaggagcttcattgtccagtttgagtgttttttgttttttgtttgagaaagggtcttactgcatcgcccaggctggagtgcagtggcacaatcactgctcattgcagcctcaacatcctgggctcaagcgatcctcctacctcagcctcccaagtagttagaaccacaggtgcgtgctaccccacccagcaaatttaatttttttttttttttttttagagacagggtctccctatgttgctcaatctcgtctctaactcctgggatcaagcgatcttcctacgttggcctcccaaagtgctgggattataggtgtgagccaccatgcctggcctgtgttcatctttcatgtgattgtttttgcaagatagaaaggcaaaagttagtagtagcaggattgacctttgtaatatgatcttaaaaatattgtctcctaatttgcatatggaagaatctcaactaaataacctccttttaaccagttcactaaacaaaccaaagttctccctttttcctataaaacatgcttctaatgcaggttagaggctcttggcaagtaggctactgtccagcatgtccaaggtcctgctctcccatctttgagtgctatgtttactaagagtctattaggttcattcccaggcttgtttatgctcactgtatttcttattgtcattatgtaccttaatgtaatgtgagtcagaaaaaaacatatttctgtgaagactggatagaacgctttggaaagatttgataaaagcaagttactaaaaagagtgttggcagacctgtaacaaaaaattgacaaaagttatggtctatgtggaacctgccctcagaatgttcttttagtttcccaaggcttcacattctgtgttaaagaaacaaactgaaattcatggacaatgcattgcgagtgtgatttatacaagaaaggcaatctagacgtcttagcaatggaatgtaaccatgttttttaaaagcctaagtcatatatcaaatgattgtcaaaacgataacccaaaacaatgtataggttttgtgttaaaataaaatgaaatatttaaagtatggatcgtttttaatcatttctgaattcaagccctacctatttgtcccaattgcttctgatataaggactttcacataattacttttcctgtttgccattttcccatttccccttccacagacatactgtgctacctttaagtgcgaggtctttcacatatctttttttatccaattacctcattcaacaatatttattttaaaaaattttttaaagagatgggatctgcctatgttgcccaggttggtctcaaactcctgggttcaagcagtcctcccaccttggcctcccaaagtgctgggttacaggcatgagccaacacatctggccctctacaaatatttcctggacaattactatgtgccaagcactggcctgggtcctgggcatagaccaacgaactgtagagccaacagtagagaaaacagactttaaataatgagagaatcatcatttaataataactgtggtgtgcgttatggagaaattcagagttcactctgtttttatataactcagacctgggatggatttcctacttctgaatggtaaagtaagtgtctgagtagatgagccaccagccttcttccttgtattctatttttaatgcagtttctttgggccaaatgatcaccgtcatccttaccagctccagactgtgattccatagtggctggattgccttggagcttagagcaggttatttcagtttactccatggatattctctccactcttctccacttcatgatggttaatattgagtgtcaacttgattggattgaaggatgcaaattattgttcctgggagtgtctatgaaggtgttgccaaaggagattaacatttgagtcagtggactggggaaggcagactcaccctcaatctgggtgggcacaatttaatcagctgccagcgtaaccacaataaaagcaggcaaagaacgtgaaaagactagactcgtttagtcttctggcctacatctttctcctgtgctggatgcttcctgccctcaaacatcagactccaagttcttcaattctggcttccctgatcctcagcttgcagacggcctattgtgggacctcatcctgtgaccgtgagtcaacgctcgttaataaactcccttttatatatacatctatcctattaattccatccctctagagaaccctaagatatctctattctgttccccaagaggtggacctttatggacccaatcaactgcctccctcaccctgtgcctccaggtggcttggaaagcaggaggcgatgaaggctgtgggttttgttctctatctatctatctgcagaattgtcaagtgttggcagcctctctcttggcaaagccaccacccttgtcaagcagcactctctttatagctactctctaggtcagggattgtcaaacaaggcccagactaaggggtggcaagcaaagtgccttggaagccaaggttaccctcggagttatgcaaatgcagggcctgagagtccattctttcttaaattttgcaccctggacacctcacttgccttacccttgtcccggccttgatgaaaacttcctttgtaaaggactagagaataaatattttaggctttgtaggacacacagtttctgatgcaagtattcaattctgcttttgaaatgtgagggcagtcatagacaatatgtaaacaaatgggcacagctgtgtttcaataaaactttattttcagaaacaggtacagggccaggtttgatccatgggccatggtttgccaacacctgctctaagtgatacagttccccatggttgcttactctgggacagcacggtcccttgtggtttcctttaatcctgctcacatctttgcaaaaagcccctttactaagctcccctcgaattatccaaattgagtgtgccatctgtttcttgctaggaccctaattgatgcacttggctttgcatttctccaacacaggctttaattgcaaagtaacaacaacaaaaaaaacaagaaaatggagactacaaccaattactctgtagcagggaagtggtggtgtttacatgtattcatccattcccaaatatgatagaatatttcacaaagttgcagtacactcttaaagttgatgggagctgaaataaataagcctttttttttcccctggggcctgcactctcaaccctgctgccctacttgtcctcttctaatggaatggaggtctattctaattaaacaagaaaacacatcttttcacaaagggagaacggaggacattgtactctgttcctcaaagaaagcatgctttaataagaatccttttataatccctgcttttatttgtagcttcccaaagcttttgctggtaataaaaggattgtaaagttgacaaatataagcagggacaaagaagtttggggagataatgctctagtgaatgggctagtatttgaaagtttattgatcctgcaacacctagaagatgatcacgaagagccaatgctgccatcaatcagggtgatatttctattacaaagtaaaagcccttccaggcagggctatccatttctcatatcatgtaagaattatgctttgtgctgagaaaaataattcaggccaaaatgcagggcttggcatctgaagatctggattcaaatctggcttcatagaacttctaacctacttctttatcatatggaagcagcaaatagagctgtaacaaatgcaatgtgttctgttgtggggttgcagacatctgctgtcattccaccactatgactatcacaagtagcatccattactcctggaaaaagccacccattttcactgaggttccctcactctcacacttaaagcctcacactttccacttctggtggagctccatctctgtggcaagggtggaacatgtgaccaagcctagaccaattaatatactgtataccttgggcagagagatttgtttaattataggcaaatgatccagttacagccaatgagactttctaggaaagtgattagacaggctgtcctttctcactaggcttgaatgtgagggctggaactgctgcagccattgtggatatatggggagcctgaggctgaagtcaacagagtagaaggatgacattgttcaagccccacatagagccatgcctaaagctggtattggttgggagaaaatatagacaagttttcacaatttcctttatctaaaccctttgtaatgtgactttgcagattctcccattgagaacattaagagatggaaactacttcctactccttgaatatggactgctcttgtagcttgctttgacccatgcattccagtggaagtgatggtgtgcacttttgctgtcagaatcctgcaccatgcagaaaagcctaggtgaacctgctggaggatgagagacaatgtggatgagagccaaggcatcccagccaacagccagaagtagtgttgcctagctgacccacagctaactgcagatcaataagggagcccagttcagaccagaagaaacatccaactgagcccagcttaaattgctgacttgcagattttctgcgaaataaacagttgttttaagctactgtatgttgaggtgatttattattcatcaataactgatacacatcccatccctgaatttttcatttatatgaaccgataattttatttttgatgtagtcagttaggaatgggttttctgtgacttgaaataaaacaattcttaacacattaaagatgtgtctttatttcccccactagatagcatcattagggattctgtcttatccttatccaaagcacttgccctatctggatccaaagctgagtgactgatcaataactacttagtgattgactatgtcatattgacagattcacaaatcacattttgaagagcaaagaatatatttgctcttgagtctccaatgtcatctgtagggcaattgtactttatttggcttagagcctcaacagaaatttggaagatacaaggtgcttctccaaccatcacaatagctgaaaacaaatacatctgagacattgtatctaaggaacccaactctgccatctccctcaagggaacccagaaaaatctgggctttaatcctaccctccttgctataaaaacctgaaatgatgtcatctttttctttctaaaataaaataaaggcatgtagtgcttctaggcaagaggtctagatgtatctgcctttcctgtcctccaggacaccatccacttattgctacaacttgctgcattattcctgcaatttattttcttctttttgcacaaaaaaaaaaggaagaaaaaactaaagcagggattccatagaattcatcctctactttagtaaaaaggtagaatccacctgacctattctgtatgtcagagtggaatccagcagccatttctctccaattattgaaaaattgtgtgtgtgtgtgtgtgcgtgtgtgcgtgtgtgtgtgtgtgtagagagagagagaaaaaaaaggtagatatacaccagcatttttattcttaaataagaggataatcaggcttactcccacccaagtgagtttacactttaaattcttaaatggctggtttttttttccctcagatctgcagacgaaggtccaaaggaagaagattttgttgaagactgaaaagagaaatatacttggtggacagagataactcctttcagcacagaggtcaggtaagggaatcacagactgacatttacccttctctctgaactctcccaggcctcaacagctcctaatctttcctttcttcattctccctcctcctcattcacatctctttccccaagaagactcaagaaaaagtccaagataagaaattccatttataagaaaaggaaggcacaggttaatttggtttatctcctccaacctcaacaatggatgtgagttctgacaattgcttgtggccagaaccaatcaaactttccttcctcattagatgacaaggctgcagcatgaagaagatgccatattgtggaattgggtgtcaaggagaagacattcgaaggtgcatgaacagagtgaaggtggtgcaagcaagtgcagaggaggtggggagactggagtgctgggaaaagggtcagcagaagctagcaaacatgaaagacacaacacaaaacaagcagttgtgcaaattaggatgataaggggggagtggtcaaaggtgtgtttctcatggctcaaccagtgcaaactcagtcttatgaccctacatgactgactgcacctggcctctgcatcccttcagcctcttccccaaaaggactcctccttattgccagccacacaagccttccttcatttcctctaacaggccagacttgttcctggctcccggtcttggcattcactgttttttctgccatagctggctccttcccaaacatggaatctcagcccaaacaatatctcctctgagacaccagccttgatcacctttatacaatacagatctgctcctgcccatcactttctatctcatgaccctacaatgtgccccttcgtgatagctcacaccagctaaaaccatcttgtttgtggattgctcacttgccccctgcctgtctctagctctaggaggtcggctccctgtcactgtctggtctactgtgtcgtaccacccatgcccagcacctagccttgcacattataggacttaatgcaaacttcttgactgaaagaaaaatgaagaaactcagccttgggagagaaaacaaccattaaatgattatttaataaaatagagtattaatctattttatattaattattaatttagttcataaaatattaattgtctttacaacgaatgtgtccttagtcattggtgtttgtgctttgtgaatcttcacatcccatggggccagtaatgggggtgggggtgtagtgtgggaagtggggcagggaaccaatctggcctgcaggggttacctgccccatgggtgcctgcaggcttcatgtggtttcagtggcatggcaccctgatgggaagagtggctcagaattggtggagtatggtgataataaaaagcaaaacaacttttagttgattattgttttttaattctataccgactatggccccttcactacctgcaccctgtgaggagagttttcccacatctgtcccactcccaccctcacactccactggttttttcccccttcttgagtgaacacacgcactggaagagcaggtgtccttgaagggaggaccagctccatgacaggacacatgtcctcccaaacacaaacatacgcaaacacagaacaaattagaatctatcctggctcctgagaacacttgctctcaggccactaaagcaccttgataataaattgtcctgtgcctgctacctctgtctccacctgctcctggcagaaatctcttccctcattccagccactctctggagggcaggcagggttctgtttatccatgtccacaggccatgccaaccaatggctcatggcttccccacttccaggaaagccacatggcccaggatacaatagagtagggaatgagtaggaggagaactggtctctttatcccagcaatatggaccttgtgatggaaagagacctaaagacagtcacatttacttctcatctctacccgggccctttcacacctaacagagtatggcaattggtacatggatagcactgagctgagtgacccatcattacttcctgccatcggacacttatgttccatcccagtagggttccctagtcacaatgaagatccagtctctatgctacctccatctgatttcttccttgatctctactccttttatcccactaacaaattttgtattattacttttcatttattcaaaagtatacatttgtttgtgcacaaaaaaatacaatttctttttttgtatggttcagatttaaatcccacatacatgatatatgcaagcaagttgctttgctggggagatttgtgaaatttcctcacactcatgttgtaagctactttgtggtcctcatctcttttcacatggacagcactcttacagaaattaagaactttatttttttgtagaaaaccagatgtaagcccattgaaaggagaaaccttggctatctttgtcattataataaatctagtatgaaattgttggttagaaaatattaaatgaaaggaagacagagaaggagggaagaggaaggtaagaagggaggagggagctagagaaaagggggcgggagggagaaaagaaattaagttgtcctgaaactcaactgctaggacagttactgtgagtaaagaggaaataacagtttatagaaaaatatgtttcttattttgaaaaaaaaaagtttggaataaaacaaaaaatacaaagacccctattttttagaattatgaggagttggaggagtcacttaaatgtttgttaaacatcactacttctacctataaaggggaaaatacttataaagggaaaattttaactagagtgatatttgggacttaaaatgtctggtaccaattttatccatttctcatcccagagcaggaagtttgcattgtttatgactgaatattcaagtatctttcccccactaggatttactgtaggttcgtggtagtgacaggggatttaaatgaaaatttttaggtctcggtcagtaaaacgacctgtgtcagcctagagaaagaggaagagaggagacagggttgacccagcgctggacgagtctgaagatgtgcatggggagaaaataattgagctcatgtgacccaaagtcaagtgtactctgggtttaggtctcagctgcagtatttatcggactgtgaggatcatttcagctccctgagcctcagtttactactcatctacagaatggaattaataacattaaatgagatgatgcatcactagacatttagctcagatcatggcatgtattacatatgccacaactgttagctagtgttatgataatctttgttattattatggtcaagatgcctactgcaagactaaatatatatcaaatgttttctgttgagcatacaacttgagcatattttccaacttaccttacagttccatgtacccacgtggctggattctgaacaatgaccacttccagaccggacttacaaaatctctctccacactctctcatttccctcatcttccgagaagatccaatggaggacttcaaagtcctagaagatggcaaagcgacaagacagaaagaatgtcatccatgaatgaccacgtggagcagagctcccactccctgccctgctgtcccacattgcacaagaatgtgacatagaaacacaatttcatttctcttatgtcacggaaatttgaggactcttccttatagttgctagtattaattaccctagctaatgccaaggaagaccaaaaagtctttccaatgctttgtaaggactaaccagaagacacacataaacacagctttgctcaaattcatttttgtcacaaatcacttcttcagggcctcagaatgtgaattccagctgctctgtcaaccaatgtctagaaacatttttgctgttgagtgcaaagtcttcattcatgagggtgctctccaaacgcattcattccatggtgttcatttcttggtatagaagcatatgaggaacagcacactccctggatctatatactacattacctgtattaataagaatgctaggctgtacccattcattatctaccagcccgccagacaaaatgattcaccctagacttacaggttgaagtcaattgagtatcctttgaactggagggagaggcgcttccataaagtggtgagagctttatggcagaaagttctattaatgaccccacttcagtgcttatcaggagcaggccatataatcccttcccggaattacatgctggtcggttcagataaataacccagatctctgtcatttataatccaaaacgttaacaattaatttccaaaatcaatcctgaagggggctagaagcctaaatcgctaggtccaggcagaccccactccatgaatttctcatgagtcaaaatttaagcctcgaaacttgtaactaactgttgtcaagacacagctgacttaggcaatcaagacaacagtattcagcagtaacctgggtgaaacatactctaagtgtgaattaataagtcggctgacaccaggccccagatgtttcccactctggcaatatgcctgaagtgatgataagcagatttacatacttaactaatatgtttatttaaaagaaatttccaaatccaaaataacatccagatttcttatttggctgcagatcaaaagacatcctatcaataaagacagaatccaggataaaccttttcaacatctgcttaaaaccaaacaacagatttgagagttttggtaaaacttaattgcagacaaatttcactgcctcttacataaatatatattttcatgtccctggggtggttctgacctcatcaattttcgtgttcacaatacacaacattgttcttttaccagcaccaacaattctgcctttatggagaattcccatcccaaggttttctttcagactgaaggataaacagactttctctctggccaggaaaagtcgaacaaaagtcaggggcatgtgctaacacttggaatgttgtagaaaagaaaaatcgacccctctaccagtaagatctttgtttttctataaatattagaaagcatttatttggtctccatctccgataaatctggaaggagaatagaaaagtagagatgctccttgagtattacattttttttttttcttttcggtggtgttgattccccaagggagagcttagtaactggatcaaagtcttaacagatctgtgtgcttgtgaaggagatgataataggaggggtgttggccatagtagcacttatccctttagtgtgaattagttcactttcttcaggtcttagctgtctgaaattggaccatgatccttgggcactgagacatttttctcccatctggccagtgctgcaattgagctaaggatttggttcagagactcttgttgcctggggatctgtttaatgtcaagaccgatattagctggttgaccctggggtcgggtgacttagagagtcagaagtcatccatgtttgctgctgctccaccctttgaacttagtcatgataaactcctgggtccaatcctgttacagccaaaatagcaagaaaagagtggtagaagcaatcagaaaaatgcatagtaaggcaccaatgagataccatcttcctcttctcaattggcatacactttaaaatagcggtgttcagtcttttggcttccctgggccacactggaagaagaattgtcttgggccacacataaaatacactaacaccaatgataactgatgggcaaaaaaaaaaaaaaagaatcacaaaaaatttctcataatgttttaagaaagtttacaaatttgtgttgggccgcattcaaagccatcctgggccacatgcaggacaagcttgctttaaaatatcccatttgaagacagttcagatctccccagttatagatgatttatcaaataactatgtatgtatgaaattaatatatcaaaagccttaaaacatttccaacctttgaccaaaaaattgttcttctaggatgtatcttaagacataattatgatttgtgcacaaatttagctatgaaaatgtagttttacttatgatatgacaaaaatatcacaaatgacctaggtgtcaaacaatagagaccagttaattaaatgatgttccacaacttatataattattaaaaacatttaggatatttacatttaataaaacccatagtgagggcatttagattgtttctaatttggacttttatgaataaagctactatgaaattattgtatagtttttctttttataaatagatgctttcatctgttttgggtaaccctccatgagtaaaatagctggattatatgggaaataaatgtttaactttataaagaactaccaaacttgtttccaaagtgattatatcattttacaacactgcaacaatgtatgaaaattctagttgctccacctccttctcaatacttggtttcccaatcattttaattttagccattctggcaggtgtgtagtgatatgtcattatcgttttaatttacattcccctaataattaagaatgtgagcatcttttcatgtgcttattctccacttgggtattttattttgtgagttgactattcaaatcttcttttcattttttattaggtcatttgtcttattgagttgtaggagttcttcatatattctggatataagccctttgtcagatgttttgcaaatattatctcccaacctgtggttttcattttcattttgtttatagtttctttctttctttctttctttctttctttctttctttctttctttctttctttctttctctttctttcttttctttcttcctttatttctttttcttttcttttttttttttttggtgagacagtctcactccatcacccagactgcagtgcagtggcaccatctcacctcactgtaacctccacctgctgggttcaagtgattctcatgtagcagcccggattacaggcacttgccaccacactcagctaatttttgtatttttagtagagacagggttttgccatgctggccagcctggtctctaactcttgacctcaagagatctgcccacctcggcctctcaaagtgctgggattacaggcgtgagctaccgtgactggcttcatcatagtatctttctaagtacagaaattttgaacttaagttgcttttgagaaaaagaaatgttgtggtgcttagtgaaaagattcaagagcacctgcctttcaacccctgctcagcctctgtttttttacttggctgccaactctcttgctgtgtggacttttcctcatcaatccaaccatctggtgcctactggtggaagctgtgagtgggtaaaggccaatgaggtgtcagggaacaacagtagaaacagaagccaaacctaacctgccctggagcttcccatgtgctctagaaagggaagtgccttgtctgctgaggcaccttctagtactttccagctttaaacgaagcatggatcttttccttcacaattaacaaaagcccttgttcaggtatgtttggcacgtgctgtcatgcttctttgtcacaagcaaaacaagaaactagggatccttcaaaattctattagaaaatagacctttcaggaaatgttgcagaattggctttctgggatatgggaccaaatattccttcattccctgagaattgacttctttctgctgtatgctccaaatgaactccatatatacatacctcatcatgcctctaaatttttagggcacacatttctttacatgtctacctcctgcaattacactggaagccccttgaaagcatgaccatgttttgtacatctttgtatgtatagtgcctgtcttagtccatttctattgctgtaagaaaataccttaaatctggtaaattataaatcatagagatttatttctcacaatcctgacgactgggaagtccaacatcagggcaccagaagatttgatgtctgcagagggctgtctctgctttcaagatggtgccttcttggtgcatcctcacatagcaaaatgacagagagtaaaaaggactaaacagacttctttaagatgttttacaagggcactaattccattcataagggcagggcctcataacctaatcacctcctaatggccccacctcttaataccattgcattggtgattaagttttacaaattttagaaagacacaaacattcaaaccatagcagtgcctaccacagaactgatacagaatgagaactcaatggatactacttgaatggctgaatgaatgaagtgaattaattgttcgtatccccaaatctgagagatatctctctgtttaataatctattttgttaaaactttgctctggagttggacagacctgggatcacatctcagctccagttcttgctagctgtgtaactgagacaaagttctcagcttcctttgagtcccatggcttattaaaggggaacaatggttataattataataactacctcatgagtgtgtatggactcaatgagttaacacaaagtgtgtaagccagtgcctggttcttagtaagtgctctgtaaatgttagctttggttatttacatgctgtgttttatgtgacccatttcttatgaaataatgtttgctacaagtagaagatgatggtgatggccaaatgtgcattatatattttagtgttcataacaaccagttattattatccttttttcagttgtaattgaagtgacatgcgtgaggtcacaaagccagtgataaagctgggattcagactcaggctgaccatactttaatctctaaacatagtcactctgtcccttcacacaggaaagcattctctgatttggaggagagaattcctgtagccaaggatggtggcataagccatagacatggggctgtcaaattgcttagcctagaagaacaagagtcagccaactttggggactccctgttctatccccctccctcttatggcaataggattaagttctgtgtattagtccattttcatgaggctgataaagacataccaaagactgggtaatttacttaaaaaaaaaaaaaaagaggtttaatggactcagagttccacatggctggggaggcctcacaatcatggcagaagacaaaagtcacatcttacatggtggcagacaagagagaatgagagccaagtgaaagggggatctctctttataaaaccatcagatctcgtgagacttactcactaccatgagaacagtatgaggaaactgtcccccatgattcatttctctcccaccaggtccctcccacaacacttaggaattatgggatctacaattcaagatgagatttgggtggggacacagccaaatcatatcaagtatattgcagaaaacatgaataacagtggctaaaacaagatacaattttttttctcttacataagaaaagagcagcctaaagcttatttagcctaaagctggtatggtgggttccagtccacaggaccagggttctacttttttgctccttgaaaataagcatccaggagaaatgctgttcttcctcacggggcctgagattctcctctcctaccaagtgtcacctttctgctggtctgcagaaactcctctgtctcttcaggcagcagcaccagatacaccaagcagaccaaaacaacatcacaaaggctctgaaaattaaactgtctttgtaaccacagtccacgaaactatgccaagacctccgtgctaaacctaaacaggctgactgcctgctaaaataaaagattaaaacaggatccagagtctcctaatataatagccaaaatgttcagtatacagtaaaaagccatgacactgagaaccaagcaaaacacaagtgaactaaaaaagaccatcaactgacagccatgctcagatgaatcagatgctggaattgcctaacaaggattttaaaggagacagaaatgcttcaacaatcaattacacattctcttgaaacaaataaaaaaaatataaaatctgggcaaaaaatagtttttttatttttttaaaaaaaggaaaccacagaactctaaaatatgcagaaactctaaaataaaacagaaatagaaagggcatgtctcctagccagatcagccattcaaagaagtgcagaagaactattcaatgattttcacctaatttccttggctactttccagctgcaaggaatgctgggaaatgtagtctttaagcaggcacaccaatgatagactggatttttgtttaccaggtaaaaggaaagaatatgtatcgggttactaagctacccaggtctgcccctgacacaaagggctcatacattgggaacaaaatcaaggctctgggagtttacattggaatatgatcttactgtggataagaccaaagaaatcctgtgcacacggatgaggattataaatcctggttcaggctggcatgatccacagttgccacgattcctgtatgcctgcagaaagacaacttaatgtggcctaatgttactaacatctcgacaggctcaccttttgagggaaggccatttaattacctgattaatttgctcatcttaattaacttgttcctattgttgataaccttgtattgattgctctgctggtagtaattagaaaactggtaaagaagtaaggagagaagcccaaagaggggggcagcacagcacacagattgtcttcctgtctcctaagaatctactgtgtaggctgtgagcttagcatatttgcttctctttttaggtaaatatgttatagacacaaatgatgttctctgttgattatttataaccactccacgttccttcttagagtatctgcccttttcattttttcaaagactcagctctcatgagccccacatgacctgctacccccatcaaccacagctgattggataagcacctgacctagactgtgccagtcacatttgattgtttcagatttctgaatcagagcatgaagaaacagtaccagttaactttggatgctgaaactataggacgatgcagacttggaaggtggacaaggtagctatttcgtgatagccaccatggaccctgtattcccaccaacaatcagagagcacttgtcagttaagttcaccagactcctaatggcattatagttctcccaccaaacttcttgccccagggtgtatggactatctctgattatttgatagaagtttccctgtacattagctgactcaagttcattgccattttttgcaacccaaagagtcccttctataacaaagcactctcatcattgacttctctctatcttagttttaagtgtctctggtgaaaatgcaaatttaaaggacctattaaaaacaacagagtgggagttgctatcaaataaagtataaactgctaaactgctaaggcctctatcaagaccaatgctgtgctaactctttccaacccagtaaaacgaggctttattgtccaccactaacatgtcaccacctcccctattgacttcagtcctttgggaccccctaactctcataccccacccagcttccgagaactctattgcctcccagaagtaatcatagtcaacacttagtggtagttattttggcttgaggccatggccttgcaaagctggtattgaagtgatgctctcatatctaatttgactgaattgagtgttccagtaagactggtgatgttttccttgcttcagggtccatcctaatgagcaaatgctgccttactttcacagaaggctgcaaatgtagacttgtaaagaaaatctttacaaacacttcttggtgtcttagagatcacccaacacaattacacatggggatatgctgggaagtactaaaataaacctgatttatccttcacatatgcctttcatttgctattgtggcttcaaagctcaaaaaaaatagagagatgtttctctaaactttcactatttattaactgagaatgtaacggaagtgaaacagccaacaaaaattaaaagcagcttactcagggatacagagtcatgactatgtggagccccatatctttacttctttgtctaaagtcctcataagcactgtctgagtttgcatgagtcctacaaactagaacaactcccaacttcattgagaagaatgaggacatctttgcaaagaaggcagaggaaagccacatgctacttgtaacactttgatggctacccagtgccaaggtttttttttttttttttaacaatcctgacagcagaaatctggaagcttgatcctagaagactgaagagcccccaacatgaccctgaactctcacataagagaggaactcttagttcgtttgccattaagagaaaaccacagactaggtcatttataatgaacagaaatttattttctcacagttctaaatgctgggacatccaagatcatgggacccgcatccgacaagagtgttctagctgcatcattccatggtggaatggccagagagggtgagggagagaagcaaaagggggccaaactcatccctctgtaaggaactccctcctgagataacagcattaatcattcatcagggcagagccctcacggcctaatcaccttttaaaggtcctatctcttaatactgttataattaaacttcaacctacatttgggagggaacaaacattcaagccacagtagaaaccagcttctttttctgttgttaaagcaacactttttttcttctctgttacttgcagcctaaccagatcctaataaatacacaagcttctatgtcgacaaatatgtctacaatgctgtttaaatggcagcatggtattctaggatagggaagggccaccatttactcaaccagcccattcttggaggatgtttaagctatttccaatattttgctatgttcaacaaagtattaaggatctccctagtagctaagcacccatgtaaatccttagatatcttaggataaacctgtaaactagtttatgtgtcctagtttagctgggcccagctggaccatcacacatatttaaatttttttgaaacttattgccaacatgcatctccagagaggtagtacaccaaattatgctcctacaaaagttgcagaaacttgcctgtctatccatactgtcacccacaatggtgattattactttttatctgtgacaatttgatttatgctgggattttgcagccagcaccaaggtagcatgttgtctctcagtctaataataatttccctctccctcatcatcatcaccagcactaccaatttctctgcactatgggtactaacatcaaaaatgttcccatcacatttacttcattttgaggaaaagaacagttactaccattcattcattcactcatcacaccaatattactgaatcctaccctggaccagcattgttatggtagatcttgataatatagtgacagacaataccatccttcccaccatttacatagtagactgcctggggctcatagtctactcacatctcagcacatgagatagtcccattatcacattctcttctatctctctcacaggcactcactcaccagccatggagaacttaatgggcattaggttcttaatcctgctgggtataattggagataaccaccctggctgactgagcactataccaggctactcatggatgatttggttattagacgtaatcagagtggcccttgtatgctaaaaaggagactcagtctccatcccatggaagattacatttttctgactgccatccctcagatctagatggtattgtctccctgacatccaaatatcttcaatcatataacc